(19) United States
(12) Patent Application Publication
(10) Pub. No.: US 2026/0285924 A1
(43) Pub. Date: Sep. 24, 2026

(54) PEPTIDE THERAPEUTICS

(71) Applicant: The Regents of the University of California, Oakland, CA (US)

(72) Inventors: Prashant MALI, La Jolla, CA (US); Sami NOURREDDINE, La Jolla, CA (US)

(21) Appl. No.: 19/490,875

(22) PCT Filed: Jun. 7, 2024
(86) PCT No.: PCT/US2024/033120

§ 371 Date: Dec. 5, 2025

Related U.S. Application Data
(60) Provisional application No. 63/471,736, filed on Jun. 7, 2023.

(51) Int. Cl. C07K 14/47; A61K 38/00

(52) U.S. Cl. CPC C07K 14/4702; A61K 38/00

(57) ABSTRACT

Provided herein are, inter alia, peptides, nucleic acids, and methods of identifying and uses thereof. The peptides provided herein can, inter alia, be used as therapeutics. The peptides provided herein are, inter alia, useful for treating cancer in a subject in need thereof. Also provided herein are, inter alia, methods for screening genes or proteins encoded by genes for inhibitory or stimulatory activity in a cell.

### RELATED APPLICATION DATA

This application claims the benefit of priority under 35 U.S.C. § 119(e) of U.S. Patent Application No. 63/471,736, filed on Jun. 7, 2023, which is hereby incorporated by reference in its entirety and for all purposes.

### GOVERNMENT SUPPORT CLAUSE

This invention was made with government support under W81XWH-22-1-0401 awarded by the Department of Defense, and GM123313, CA222826, and OD032742 awarded by the National Institutes of Health. The government has certain rights in the invention.

### SEQUENCE LISTING

The material in the accompanying Sequence Listing is hereby incorporated by reference in its entirety. The accompanying file, named “048537-665001WO_SL_ST26.xml” was created on Jun. 6, 2024, and is 2,474,771 bytes in size.

### BACKGROUND

Genetic screening has rapidly become a ubiquitous tool to probe protein function and accelerate drug discovery. Libraries of genetically encoded perturbations (CRISPR-Cas9 sgRNA, siRNA, etc.) enable high throughput identification of proteins essential to cancer cells fitness and infectious organisms. However, existing screens typically fail to capture how proteins function biologically and provide little information on how to target hits therapeutically. Transposon mediated fragmentation and overexpression of cDNA has been used to identify peptide inhibitors of essential proteins in Saccharomyces cerevisiae. However, these libraries randomly generate gene fragments of various lengths hindering control of library composition, feature many out of frame fragments, and are limited in their translational relevance due to the choice of model organism. Provided herein are, inter alia, compositions and methods that address these and other problems in the art.

### BRIEF SUMMARY

In an aspect is provided a peptide comprising an amino acid sequence having at least 80% sequence identity to the amino acid sequence of any one of SEQ ID NOs: 1-2327.

In another aspect is provided isolated nucleic acid encoding the peptide provided herein including embodiments thereof.

In another aspect is provided expression vector comprising the nucleic acid provided herein including embodiments thereof.

In another aspect is provided a pharmaceutical composition comprising: (i) the peptide provided herein including embodiments thereof, the nucleic acid provided herein including embodiments thereof, or the expression vector provided herein including embodiments thereof; and (ii) a pharmaceutically acceptable excipient.

In another aspect is provided a kit comprising the peptide provided herein including embodiments thereof, the nucleic acid provided herein including embodiments thereof, the expression vector provided herein including embodiments thereof, the pharmaceutical composition provided herein including embodiments thereof, or a combination thereof.

In another aspect is provided a method of treating a cancer in a subject in need thereof, the method comprising administering to the subject a therapeutically effective amount of the peptide provided herein including embodiments thereof, thereby treating the cancer in the subject.

In another aspect is provided a method for treating a disease or condition in a subject in need thereof, the method including administering to the subject a therapeutically effective amount of the peptide provided herein including embodiments thereof, thereby treating the disease or condition in the subject.

In another aspect is provided a method for identifying a peptide or fragment thereof in a cell, wherein the peptide or fragment thereof modulates the activity of a target protein in the cell.

In another aspect is provided a method for screening genes or proteins encoded by genes for inhibitory or stimulatory activity in a cell.

### BRIEF DESCRIPTION OF THE DRAWINGS

FIGS. 1A-1B show an overview of experimental design and initial results: (A) Design of overlapping gene fragment library. Gene fragments coding for all possible overlapping 40mer peptides were computed from target gene cDNA sequences. Gene fragment sequences were then generated via chip-based oligonucleotide synthesis and cloned into a lentiviral plasmid vector. This plasmid library was in turn used to generate lentiviral particles via transient transfection. The lentiviral particles were then used to infect target mammalian cell lines at a low MOI to ensure only one gene fragment was expressed per cell. The cells were then grown for two weeks, with genomic DNA extracted at day 3 and day 14. Next, gene fragments were PCR amplified from genomic DNA and sequenced to track fragment abundances and calculate log, enrichment and depletion. Gene fragments were mapped back to target gene coding sequences, and each codon/amino acid was given a fitness score defined as the Z-normalized mean log, fold change of all overlapping gene fragments. (B) Resulting amino acid level fitness scores. Screening data from Hs578T and MDA-MB-231 cells shows conserved regions of fitness dependencies, as well as cell line specific fitness dependencies.

FIGS. 2A-2C. (A) Pan-Cancer library gene composition (B) Peptide modalities, genes were tiled every 6 amino acids, and every peptide is 40 amino acid long. (C) KEGG pathway enrichment of the Pan-Cancer gene list.

FIGS. 3A-3B. (A) Genetic stratification of malignant melanomas. TCGA Firehose Legacy data from cbioportal.org. (B) Mutation status of BRAF and NRAS proteins from 480 patient sample.

FIGS. 4A-4D. (A) Brightfield image of A375-wildtype (WT) and NRAS^{Q61K} (B) Protein expression of endogenous RAS and exogenous HA-NRAS mutants by western blot. (C) Growth assay on A375-WT and -NRAS^{Q61K} using CCK8 assay. (D) Cell viability dose-response to 48 hours Vemurafenib treatment (BRAF inhibitor) on A375-WT and -NRAS^{Q61K}.

FIGS. 5A-5B. (A) Pan-Cancer library coverage and mapping of the plasmid library and the screening conditions. (B) Normalized count correlation between replicates.

FIG. 6. Waterfall distribution of the Pan-Cancer peptide fitness score across A375-WT and -NRAS^{Q61K}. Cut-off criteria Fitness score<1 False Discovery Rate<5%.

FIG. 7. StringDB network of the top genes negatively affecting fitness of A375-WT and -NRAS^{Q61K} cell lines. Each genes represented display at least 5 unique peptides with a significant negative fitness score.

FIGS. 8A-8B. (A) Per position Rolling Log 2 Fold Change (LFC) in A375-WT. (B) Alphafold2 structure prediction, colored in green the peptide selected for the validation assay.

FIGS. 9A-9C. (A) Fitness screen validation assay workflow. (B) Validation assay results (C) Fitness screen results.

FIGS. 10A-10C. (A) BRAF per position fitness score profile in MDA-MB-231 [1]. (B) Validation assay results in a breast cancer cell line panel [1]. (C) BRAF per position fitness score profile in A375-WT and A375-NRAS^{Q61K}.

FIG. 11. Per position Fitness score profile of ARHGAP20 and ARHGAP26 in both A375-WT and A375-NRAS^{Q61K}.

FIGS. 12A-12D. A-to-I PepTile screen. (A) Composition of A-to-I PepTile linrary screen, and schematic description of the lentiviral expression vector. (B) Experimental workflow. A375 cells were transduced at low MOI, and then treated or not with IFNα for 48 hrs. RNA was extracted and Peptide_ADARreporter RNA species were amplified and sequenced by NGS. (C) Dot plot of A-to-I PepTile library effect on A-to-I editing with or without IFNα treatment. Red line is the mean A-to-I editing of all the 8088 peptides (>100 read counts) no treatment: 25.75% IFNα: 37.6%. (D) Dot plot of A-to-I PepTile library fitness effect upon IFNα for 48 hrs.

FIGS. 13A-13D show Pan-Cancer PiTile Workflow. (A) The Pan-Cancer peptide library is composed of 1081 proteins tiled into 40-mer peptides at every 6 amino acids of the parental protein. The DNA-encoded library is cloned into lentiviral vectors and packaged into lentivirus. (B) A375 cell line was transduced with the Pan-Cancer PiTile library. Genomic DNA was then harvested at Day2 and Day 14 post-transduction and cell populations abundance expressing the different peptides were quantified using Next-Generation Sequencing (NGS). Peptide fitness score was then calculated by the abundance of each peptide at Day2 versus Day 14. Then the fitness signal is mapped over the original amino acid positions of the derived protein. (C) Coverage of the plasmid Pan-Cancer library and peptide-amplicons from Day2 and Day 14 replicated was determined by NGS. (D) Dot plot of the experimental result obtain from screening the Pan-Cancer library in A375 cell line. 3201 peptides (3.5% of the total library) were found to drive significant fitness defects.

FIGS. 14A-14E show PiTiles derived from DNA-binding domains of MYC and SOX17 inhibit transcriptional program. (A) Experimental workflow of A375 cell line individually transduced with lentivirus expressing GFP_147, MYC_384 or SOX17_96. 5 days post-transductions, RNA was harvested for RNA sequencing. Additionally, cells were plated for a 48-hour growth assay. (B) Cell growth of A375 cell line expressing GFP_147 (control), MYC_384 and SOX17_96. MYC_384 and SOX17_96 cell lines show a cell growth defect compared to GFP_147 cell line (C) PiTile profile of MYC and SOX17 proteins, with representation of their known protein domain (top) with experimental fitness score assessed in A375. Red bar=FDR<0.05. We observe a negative peak in bHLHzip domain of MYC as well as HMG box domain of SOX17. Both domains mediated binding to DNA. (D) Principal Component Analysis of RNA-sequencing for both biological replicates of GFP_147, MYC_384 and SOX17_96 expressing cell lines. (E) A375 RNAseq data was analyzed using Gene Set Enrichment Analysis. For MYC_384 versus GFP_147 over ODONNEL_TARGET_OF_MYC_and_TFRC. For SOX17_96 versus GFP_147 over PRANMOOJAGO_SOX4. Overexpression of these peptides lead to downregulation of their respective transcriptional programs.

FIGS. 15A-15C show PiTile screen identify peptides derived from A375 essential genes. (A) Waterfall plot of genome-wide CRISPR knock-out screen in A375 ordered by descending fitness score effect. In red, genes for which PiTile screen identified significant 40-mer peptides affecting A375 cell fitness. (B) 48-hour cell growth validation assays of 4 peptides derived from A375 specific essential genes. (C) Global PiTile profiles of these A375 essential genes. We observe that many peptides negatively affecting growth map on region that mediates protein-protein interactions. Red bar=FDR<0.05. We can observe Peptides peak around known interacting regions such as MEK-RAF interaction helix for MAP2K1 profile, ERK-importin interaction (MAPK insert) on MAPK1 profile, TBM, Axin-TP53, Axin-GSK3B for Axin profile, and BRAF-14-3-3 interaction for BRAF profile.

FIGS. 16A-16G show design of RAF1 derived staple peptide and MAPK inhibition. (A) PiTile profile of RAF1 protein in A375 cell line. We observe significant peptides deriving from the Ras-Binding Domain (RBD) ad Kinase Domain. (B) Crystal structure of RAF1 RBD-CRD in grey interacting with KRAS in purple (PDB:6XHA). Highlighted in green the interacting helix that was identify as a hit in PiTile screen. (C) Amino Acid sequence of the RAF1 region of interest identified via PiTile screen. In green, residues predicted to fold as an αhelix. This helical region was then used to design hydrocarbon staple. (D) The staple RAF1 and TAT-Flag peptides were tested in 48-hour cell growth assay in A375 and MDA-MB-231 cell line. The staple RAF1 peptide led to a complete cell death in both cell lines with an IC50 of 16 to 21 μM respectively in A375 and MDA-MB-231. (E) A375 cell line was treated with either PBS or RAF1 stapled peptide at 10 μM for 24 hours. RNA was then extracted, and RNA-sequencing libraries were prepared. (F) Volcano plot of quantified gene expression of cell treated with stapled RAF1 versus PBS control. (G) Gene Set Enrichment Analysis of A375 treated with stapled RAF1 versus PBS control. We found that RAS_Pathway (NES=−1.65), RAF_Activation (NES=−1.48), MAPK_Pathway (NES=−1.23) were downregulated in cells treated with stapled RAF1 peptide.

FIGS. 17A-17F show A-to-I RNA Editing PiTile (REPTILE) screen in A375. (A) A-to-I RNA Editing PiTile (REPTILE) library design consist of fragmentation of all known ADAR1, ADAR2 interactors and all human double stranded RNA-binding protein into 40 amino acid long peptide. This library was then cloned into a lentivirus vector that also encode a DNA-encoded RNA loop reporter (A-to-I Rep) that is edited by ADAR proteins in cells at the RNA level. When the construct is expressed, the RNA loop gets edited on a single Adenosine, while the 40-mer peptide get expressed at the protein level. Then we can measure the impact of the peptide expression on the editing rate of the RNA loop reporter by sequencing (NGS and Sanger). (B) Experimental workflow of REPTILE screen in A375 cell line. Cells were transduced with the REPTILE library, and then treated for 48 hours with IFNα. Following the treatment, RNA was harvested, converted into cDNA and amplicon library containing peptide Identity (barcode) linked to the RNA loop reporter were prepared for NGS. (C) Result of the A375 REPTILE screen in the no treatment condition. Each dot represents a unique peptide that is associated with an A-to-I editing rate that was measured on the RNA loop. (D) Result of the A375 REPTILE screen in the IFNα condition. Each dot represents a unique peptide that is associated with an A-to-I editing rate that was measured on the RNA loop. Red line represent the A-to-I editing mean, Dash blue line represent 2 times the standard deviation of the % A-to-I editing across all the datasets. This cutoff was used to defined peptides hits that modulate RNA A-to-I editing. (E) Peptide A-to-I Inhibitor Hits intersection between no treatment (% A-to-I editing<15%) and IFNα (% A-to-I editing<27%) conditions. (F) Peptide A-to-I Enhancers Hits intersection between no treatment (% A-to-I editing>36%) and IFNα (% A-to-I editing>47%) conditions.

FIGS. 18A-18G show peptide hits validation from the REPTILE screen. (A) Schematic workflow of the validation in A375 cell line. Cells were plated and then transduced with purified lentivirus expressing peptides selected for validation, we picked 11 A-to-I inhibitors and enhancers that were commonly identified as hits in the no treatment and IFNα REPTILE screen. 5 days after viral transductions, RNA was harvested, and cells were also plated for a 48-hour growth assay. (B) A-to-I editing quantifications of the RNA loop by Sanger sequencing in each individual conditions expressing peptides. Gray dashed line represents the mean A-to-I editing in the GFP_30 peptide expressing condition which is the control for basal RNA editing of the RNA loop. (C) % A-to-I correlation between Sanger sequencing (low throughput validation) versus NGS (high throughput pooled screen) measurements. (D) Cell growth measurement in A375 when expressing the REPTILE peptide hits. Gray dashed line represents the mean growth in the GFP_30 peptide expressing condition. (E) Correlation between % of A-to-I editing on the RNA loop versus cell growth in each peptide expressing condition. We observe a negative correlation, when the % of A-to-I increases, the cell growth decrease. Thus, peptides triggering increase of RNA editing affect negatively cell fitness of the A375 cell line. Gray dashed lines represent mean % A-to-I editing and cell growth of the GFP_30 peptide expressing condition. (F) REPTILE profile of ADAR (ADAR1) and ADARB1 (ADAR2) proteins. % A-to-I editing on the RNA loop obtain for every peptides was aligned according to the start position were the peptide was derived from. Red line represents the mean % A-to-I editing at basal level, dash line represents 2 standard deviations. The dashed circle highlights hot spot region were peptides increased RNA editing. These regions derived from ADAR and ADARB1 deaminase domain. (G) Sanger sequencing trace of the edited A on the RNA loop in cells expressing GFP_30, or ADAR_231 (position 920 on REPTILE profile) or ADARB1_126 (position 500 on REPTILE profile).

FIGS. 19A-19E show A-to-I editing modulated by ADARB1 derived peptides involved residues in ADAR dimerization. (A) Crystal structure of ADARB1 deaminase domain (PDB: 6VFF) interacting with double stranded RNA. In purple and pink ADARB1 deaminase domain, in green the peptide we identified with REPTILE screen, in gold and blue the double stranded RNA. We observe that the ADARB1 derived peptides identify via REPTILE screen are involve in ADARB1 dimerization and RNA contact. (B) % of A-to-I editing on RNA loop across successive peptide from ADARB1 deaminase domain out of REPTILE screen. Green bar show peptide that increased % of A-to-I editing. (C) Alphafold representation of the 40 amino acid long peptide ADARB1_126 (derived from position 500 of ADARB1), in red amino acid residues mapped to be involved in protein, RNA, and inositol interaction. (D) Amino acid sequence of the ADARB1_126 peptide and mutations that were designed (SEQ ID NOS: 2355-2364). In red residues involved in protein, RNA, and inositol. In purple alanine mutations that we designed on the wild type peptide. (E) We generated A375 cell line expressing ADARB1_126 wild type (WT) and the various designed mutant. RNA was extracted and we quantified by sanger sequencing RNA editing on the ADAR reporter RNA loop. We found that ADARB1_T501A, W502A, D503A, R510A and 8A significantly affect RNA editing. These residues are known to mediate ADAR dimerization and RNA binding of the edited baser (R510). These results suggest that the mechanism involving the activity of this peptide is related to ADAR dimerization and RNA binding.

FIGS. 20A-20F show ADARB1_126 expression trigger a strong pro-inflammatory response and decrease cell cycle. (A) Experimental workflow. GFP_30 and ADARB1_126 A375 cell lines were generated with lentivirus. RNA was then extracted and transcriptomic analysis was performed, gene expression was measured by comparing ADARB1_126 to GFP_30 using DEseq2 package. A-to-I editing was also quantified on all editing sites across the transcriptome using REDItools and a2iediting packages. (B) Volcano plot of gene expression quantified via RNA sequencing when ADARB1_126 or GFP_30 is overexpressed in A375 cells. (C) Reactome enrichment of differentially expressed genes between ADARB1_126 and GFP_30. Blue bars represented downregulated genes in ADARB1_126 condition, red bars represent upregulated genes in ADARB1_126 condition. (D) Top Gene Set Analysis Enrichment (GSEA) of ADARB1_126 RNAseq. Hallmark of E2F target is massively downregulated in ADARB1_126 while Hallmark of IFN alpha response is upregulated in ADARB1_126 condition. (E) Alu Editing Index (AEI). Alu elements fold in double stranded RNA, and millions of copies exist in the genome. Due to their double stranded conformation, millions of editing sites can be quantified throughout the transcriptome and give a robust quantification of A-to-I editing transcriptome wide. ADARB1_126 expression in basal condition leads to increase of editing transcriptome wide, as similar level than cells treated with IFNα. When treated with IFNα, ADARB1_126 expressing cells shows an additional increase in editing, suggesting that this peptide act as a potent A-to-I enhancer, at similar potency than pro-inflammatory cytokines. (F) We generated A375 cell line expressing either GFP_30, ADARB1_126 wild type or ADARB1_126 8A mutants. We then extracted RNA and measured by qPCR expression of pro-inflammatory genes. While expression of ADARB1_126 WT increases expression of these pro-inflammatory mRNA, mutation of 8 key residues, abrogate the increase observed. These results suggest that ADARB1_126 inducing pro-inflammatory phenotype involved ADAR dimerization.

FIGS. 21A-21D show ADARB1_126 peptide interactome using BioID2 system. (A) We generated construct expressing the Biotin ligase (BioID2) fused to a 5GS linker and ADARB1_126 peptide. When expressed in cells and upon biotin addition, the BioID2 protein will biotinylate any protein in 20 nm radius of the protein. Then we lysed the cells and purify biotinylated protein using streptavidin beads. Biotinylated tagged proteins are then identify using mass spectrometry. (B) Replicate correlation of protein identified by mass spectrometry in BioID experiments. 5GS correspond to BioID2-5GS linker which is the control condition (to identify background) and ADARB1_126 correspond to BioID2-5GS-ADARB1 126 construct. Venn diagrams represent intersection of the unique protein identified in each replicate using mass spectrometry. (C) waterfall plot of the protein identified in BioID, we calculated log 2 fold change comparing 5GS and ADARB1 condition. Red dashed line represents the cutoff (>2 log 2 fold change) for protein identified enriched in ADARB1 126 condition. (D) Network representation of enriched proteins identified in ADARB1 126. Red circles represent known ADAR interactors. Interestingly, we found that ADARB1 126 interact known ADAR interactors, including ADAR. These results suggest that the 40 amino acid ADARB1_126 peptide can interact with ADAR and closely ADAR's interactors which result in ADARB1_126 observed activity on RNA editing and inflammation.

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds, each of which is incorporated herein in their entirety and for all purposes. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “non-naturally occurring amino acid” or “unnatural amino acid” are used herein according to their plain ordinary meaning and refer to amino acid analogs, synthetic amino acids, and amino acid mimetics which are not found in nature. The terms non-naturally occurring amino acid and unnatural amino acid are used interchangeably herein. In embodiments, the unnatural amino acid includes a D-amino acid. In embodiments, the unnatural amino acid is a D-amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., Ras) in other proteins with different numbering systems. For example, by performing a simple sequence alignment with a protein (e.g., Ras) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “specifically (or selectively) binds to” when referring to a protein or peptide, refers to a binding reaction that is determinative of the presence of the protein, often in a heterogeneous population of proteins and other biologics. Thus, under designated immunoassay conditions, the specified proteins bind to a particular protein at least two times the background and more typically more than 10 to 100 times background.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g. a first linker or second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

A “ligand” refers to an agent, e.g., a polypeptide or other molecule, capable of binding to a specific protein or fragment thereof.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “stabilization moiety” is used herein according to its plain ordinary meaning and refers to a peptide modification which increases the stability of a peptide to which it is attached relative to the absence of the stabilization moiety. In embodiments, the increase in stability of a peptide includes an increase in the half-life of the peptide. In embodiments, the increase in stability of a includes a decrease in the degradation of the peptide. In embodiments, the increase in stability includes a decrease in the elimination of the peptide. In embodiments, the stabilization moiety prevents hydrolysis or proteolysis of an amide bond of the peptide. In embodiments, the stabilization moiety includes a protein secondary structural motif. In embodiments, the stabilization moiety includes a protein tertiary structural motif. In embodiments, the stabilization moiety increases cell permeability relative to the absence of the stabilization moiety. Peptide modifications to increase stability are well known in the art (Fetse et al., Trends Pharmacol Sci, 2023 July; 44 (7): 425-441; Barman et al., Int J Pept Res Ther, 2023; 29 (4): 61, each of which is incorporated herein in their entirety and for all purposes). N-terminal modifications to peptides are well known in the art (Jiang et al., Chinese Chem Letters, 2022 January; 33 (1): 80-88, which is incorporated herein in its entirety and for all purposes). C-terminal modifications to peptides are well known in the art (Arbour et al., Org Biomol Chem, 2020 Sep. 30; 18 (37): 7253-7272, which is incorporated herein in its entirety and for all purposes).

The term “protein secondary structural motif” is used herein according to its plain ordinary meaning and refers to a feature of a peptide that confers a spatial conformation or structure to the amino acid sequence of the peptide. In embodiments, the protein secondary structural motif includes a hydrogen bond between an amino hydrogen and a carboxyl oxygen atom in the amino acid sequence of the peptide. In embodiments, the protein secondary structural motif includes an alpha helix or a beta sheet. In embodiments, the protein secondary structural motif includes an alpha helix. In embodiments, the protein secondary structural motif includes a beta sheet.

The term “protein tertiary structural motif” is used herein according to its plain ordinary meaning and refers to a feature of a peptide that confers a three-dimensional shape to a peptide. In embodiments, the protein tertiary structural motif includes a protein secondary structural motif.

The term “cyclization moiety” is used herein according to its plain ordinary meaning and refers to a peptide modification which allows a first amino acid in a peptide to form a covalent bond with a second amino acid in the peptide. In embodiments, the cyclization moiety includes a protein secondary structural motif.

The term “head-to-tail cyclization moiety” is used herein according to its plain ordinary meaning and refers to a peptide modification to the N-terminal amino acid or the C-terminal amino acid of a peptide, which allows the formation of a covalent bond between the N-terminal amino acid and the C-terminal amino acid of the peptide. In embodiments, the formation of a covalent bond between the N-terminal amnio acid and the C-terminal amino acid of the peptide generates a macrocycle. In embodiments, head-to-tail cyclization includes amide cyclization, sulfur-mediated cyclization, azide-alkyne cycloaddition, or enzymatic macrocyclization. In embodiments, head-to-tail cyclization includes amide cyclization. In embodiments, head-to-tail cyclization includes sulfur-mediated cyclization. In embodiments, head-to-tail cyclization includes azide-alkyne cycloaddition. In embodiments, head-to-tail cyclization includes enzymatic macrocyclization. In embodiments, amide cyclization includes an N-terminal proline residue and a C-terminal N-methyl amino acid residue. In embodiments, amide cyclization includes an N-terminal N-methyl amino acid residue and a C-terminal proline residue. In embodiments, sulfur-mediated cyclization includes an N-terminal cysteine residue and a C-terminal thioester. In embodiments, azide-alkyne cycloaddition includes a N-terminal azide and a C-terminal phosphino-thioester. In embodiments, enzymatic macrocyclization includes a PatG enzyme and a heterocycle. In embodiments, the heterocycle is a N-terminal proline residue or an azoline. In embodiments, the heterocycle is a N-terminal proline residue. In embodiments, the heterocycle is an azoline. Head-to-tail cyclization moieties and techniques are well known in the art (White & Yudin, Nat Chem, 2011 Jun. 23; 3 (7): 509-24, which is incorporated herein in its entirety and for all purposes).

The term “side-chain cyclization moiety” is used herein according to its plain ordinary meaning and refers to a peptide modification to a first side chain of a first amino acid in a peptide, which allows the formation of a covalent bond between the first side chain of the first amino acid to a second side chain of a second amino acid in the peptide. In embodiments, the side-chain cyclization moiety includes a modification to an amine group, a carboxylic acid group, or a thiol group in a side chain of an amino acid in the peptide. In embodiments, the side-chain cyclization moiety includes a modification to an amine group in a side chain of an amino acid in the peptide. In embodiments, the side-chain cyclization moiety includes a modification to a carboxylic acid group in a side chain of an amino acid in the peptide. In embodiments, the side-chain cyclization moiety includes a modification to a thiol group in a side chain of an amino acid in the peptide. In embodiments, the side-chain cyclization moiety includes lactam stapling or hydrocarbon stapling. In embodiments, the side-chain cyclization moiety includes lactam stapling. In embodiments, the side-chain cyclization moiety includes hydrocarbon stapling. In embodiments, side-chain cyclization includes formation of a bond between two side chains of two different amino acid residues on the same face of a helical peptide. In embodiments, the side-chain cyclization includes stapling amino acids at a three amino acid distance within a peptide. In embodiments, a one-turn staple includes a staple (e.g., bond) between two amino acids at either a three (i/i+3) or four (i/i+4) amino acid distance. In embodiments, a two-turn staple includes a staple (e.g., bond) between two amino acids at a seven (i/i+7) amino acid distance. In embodiments, a three-turn staple includes a staple (e.g., bond) between two amino acids at an eleven (i/i+11) amino acid distance. In embodiments, the side-chain cyclization includes a lactam, a hydrocarbon, a Cu(I)-catalyzed azide-alkyne cycloaddition, a thioester, a bis-thioester, or a bis-lactam. In embodiments, the side-chain cyclization includes a lactam. In embodiments, the side-chain cyclization includes a hydrocarbon. In embodiments, the side-chain cyclization includes a Cu(I)-catalyzed azide-alkyne cycloaddition. In embodiments, the side-chain cyclization includes a thioester. In embodiments, the side-chain cyclization includes a bis-thioester. In embodiments, the side-chain cyclization includes a bis-lactam.

The term “hydrocarbon stapling” is used herein according to its plain ordinary meaning and refers to a peptide modification that forms a covalent bond between two amino acids residues in a peptide. In embodiments, the hydrocarbon stapling occurs between any two amino acid residues in a peptide. In embodiments, the hydrocarbon stapling includes ring-closing alkene metathesis of two amino acid residues in a peptide. In embodiments, the alkene metathesis includes formation of a carbon-carbon bond between two amino acid residues in a peptide. In embodiments, a peptide including a hydrocarbon stapling modification is referred to herein as a stapled peptide. In embodiments, the stapled peptide includes a protein secondary structural motif. In embodiments, the stapled peptide includes an alpha helix. In embodiments, a peptide may include more than one hydrocarbon stapling modifications. In embodiments, a peptide may include more than one hydrocarbon stapling modifications at multiple amino acid residues. In embodiments, a peptide including more than one hydrocarbon stapling modifications is referred to herein as a stitched peptide.

The term “PEGylation moiety” is used herein according to its plain ordinary meaning and refers to a polyethylene glycol (PEG) group. In embodiments, the PEGylation moiety is covalently or non-covalently attached to an amino acid residue in a peptide. In embodiments, the PEGylation moiety is covalently attached to an amino acid residue in a peptide. In embodiments, the PEGylation moiety is non-covalently attached to an amino acid residue in a peptide. In embodiments, the PEGylation moiety is attached to the N-terminus of the peptide. In embodiments, the PEGylation moiety is attached to a lysine, a cysteine, or a glycine. In embodiments, the PEGylation moiety is attached to a lysine. In embodiments, the PEGylation moiety is attached to a cysteine. In embodiments, the PEGylation moiety is attached to a glycine. In embodiments, the PEGylation moiety is attached to a lysine for non-specific PEGylation. In embodiments, the PEGylation moiety is attached to a cysteine for site-specific PEGylation including maleimide-PEG. In embodiments, the PEGylation moiety is attached to a glycine for site-specific PEGylation. In embodiments, the PEGylation moiety has a mass from about 1000 Da to about 40,000 Da. In embodiments, the PEGylation moiety has a mass from about 2000 Da to about 40,000 Da. In embodiments, the PEGylation moiety has a mass from about 3000 Da to about 40,000 Da. In embodiments, the PEGylation moiety has a mass from about 4000 Da to about 40,000 Da. In embodiments, the PEGylation moiety has a mass from about 5000 Da to about 40,000 Da. In embodiments, the PEGylation moiety has a mass from about 6000 Da to about 40,000 Da. In embodiments, the PEGylation moiety has a mass from about 7000 Da to about 40,000 Da. In embodiments, the PEGylation moiety has a mass from about 8000 Da to about 40,000 Da. In embodiments, the PEGylation moiety has a mass from about 9000 Da to about 40,000 Da. In embodiments, the PEGylation moiety has a mass from about 10,000 Da to about 40,000 Da. In embodiments, the PEGylation moiety has a mass from about 15,000 Da to about 40,000 Da. In embodiments, the PEGylation moiety has a mass from about 20,000 Da to about 40,000 Da. In embodiments, the PEGylation moiety has a mass from about 25,000 Da to about 40,000 Da. In embodiments, the PEGylation moiety has a mass from about 30,000 Da to about 40,000 Da. In embodiments, the PEGylation moiety has a mass from about 35,000 Da to about 40,000 Da. In embodiments, the PEGylation moiety includes a methoxyPEG-amine, a methoxy PEG-maleimide, or a methoxy PEG-carboxylic acid. In embodiments, the PEGylation moiety includes a methoxy PEG-amine. In embodiments, the PEGylation moiety includes a methoxyPEG-maleimide. In embodiments, the PEGylation moiety includes a methoxyPEG-carboxylic acid. Peptide PEGylation moieties and techniques are well known in the art (Belén et al., Front Pharmacol, 2019; 10:1450, which is incorporated herein in its entirety and for all purposes).

The term “glycosylation moiety” is used herein according to its plain ordinary meaning and refers to a carbohydrate group or glycan group. In embodiments, the glycosylation moiety is an N-linked glycan, an O-linked glycan, a phosphogylcan, a C-linked glycan, or a glycosylphosphatidylinositol (GPI) anchor. In embodiments, the glycosylation moiety is an N-linked glycan. In embodiments, the glycosylation moiety is an O-linked glycan. In embodiments, the glycosylation moiety is a phosphogylcan. In embodiments, the glycosylation moiety is a C-linked glycan. In embodiments, the glycosylation moiety is a glycosylphosphatidylinositol (GPI) anchor. In embodiments, the glycosylation moiety includes an α-D-galactose, a Tn antigen, a Tf antigen, an α-D-mannose, a β-D-galactose, or a β-D-glucose. In embodiments, the glycosylation moiety includes an α-D-galactose. In embodiments, the glycosylation moiety includes a Tn antigen. In embodiments, the glycosylation moiety includes a Tf antigen. In embodiments, the glycosylation moiety includes an α-D-mannose. In embodiments, the glycosylation moiety includes a β-D-galactose. In embodiments, the glycosylation moiety includes a β-D-glucose. In embodiments, the glycosylation moiety includes a Ser(alpha-D-GalNAc), a Thr(alpha-D-GalNAc), a Ser(beta-D-GalNAc), a Thr(beta-D-GalNAc), a Ser(Gal-beta(1-3)GalNAc), a Thr(Gal-beta(1-3)GalNAc), a Ser(beta-D-GlcNAc), a Thr(beta-D-GlcNAc), an Asn(beta-D-GlcNAc), Ser(beta-D-Glc), a Thr(alpha-D-Man), an Asn(alpha-D-Man), or a Ser(alpha-D-Man). In embodiments, the glycosylation moiety includes a Ser(alpha-D-GalNAc). In embodiments, the glycosylation moiety includes a Thr(alpha-D-GalNAc). In embodiments, the glycosylation moiety includes a Ser(beta-D-GalNAc). In embodiments, the glycosylation moiety includes a Thr(beta-D-GalNAc). In embodiments, the glycosylation moiety includes a Ser (Gal-beta(1-3)GalNAc). In embodiments, the glycosylation moiety includes a Thr(Gal-beta(1-3)GalNAc). In embodiments, the glycosylation moiety includes a Ser(beta-D-GlcNAc). In embodiments, the glycosylation moiety includes a Thr(beta-D-GlcNAc). In embodiments, the glycosylation moiety includes an Asn(beta-D-GlcNAc). In embodiments, the glycosylation moiety includes Ser(beta-D-Glc). In embodiments, the glycosylation moiety includes a Thr(alpha-D-Man). In embodiments, the glycosylation moiety includes an Asn(alpha-D-Man). In embodiments, the glycosylation moiety includes a Ser(alpha-D-Man). Peptide glycosylation moieties and techniques are well known in the art (Moradi et al., Chem Sci, 2016 Apr. 1; 7 (4): 2492-2500, which is incorporated herein in its entirety and for all purposes).

The term “lipidation moiety” is used herein according to its plain ordinary meaning and refers to a lipid moiety. In embodiments, the lipidation moiety includes a fatty acyl group or a polyisoprenyl group. In embodiments, the lipidation moiety includes a fatty acyl group. In embodiments, the lipidation moiety includes a polyisoprenyl group. In embodiments, the lipidation moiety includes a cysteine prenylation, a N-terminal glycine myristylation, a cysteine palmitoylation, a serine fatty acylation, or a lysine fatty acylation. In embodiments, the lipidation moiety includes a cysteine prenylation. In embodiments, the lipidation moiety includes a N-terminal glycine myristylation. In embodiments, the lipidation moiety includes a cysteine palmitoylation. In embodiments, the lipidation moiety includes a serine fatty acylation. In embodiments, the lipidation moiety includes a lysine fatty acylation. In embodiments, the lipidation moiety includes a palmitic acid (hexadecenoic acid), a myristic acid (tetradecanoic acid), a geranyl group ((2E)-3,7-dimethylocta-2,6-dien-1-yl), a farnesyl group ((2E,6E)-3,7,11-trimethyldodeca-2,6,10-trien-1-yl), or a geranylgeranyl group ((2E,4E,6E)-3,5,7,11-Tetramethyldodeca-2,4,6,10-tetraene-1-yl). In embodiments, the lipidation moiety includes a palmitic acid (hexadecenoic acid). In embodiments, the lipidation moiety includes a myristic acid (tetradecanoic acid). In embodiments, the lipidation moiety includes a geranyl group ((2E)-3,7-dimethylocta-2,6-dien-1-yl). In embodiments, the lipidation moiety includes a farnesyl group ((2E,6E)-3,7,11-trimethyldodeca-2,6,10-trien-1-yl). In embodiments, the lipidation moiety includes a geranylgeranyl group ((2E,4E,6E)-3,5,7,11-Tetramethyldodeca-2,4,6,10-tetraene-1-yl). Peptide lipidation moieties and techniques are well known in the art (Kowalczyk et al., Adv Exp Med Biol, 2017; 1030:185-227, which is incorporated herein in its entirety and for all purposes).

The term “hydroxylation moiety” is used herein according to its plain ordinary meaning and refers to a hydroxyl group (—OH). In embodiments, the hydroxylation moiety is attached to a proline, a lysine, an asparagine, an aspartate, or a histidine of a peptide.

The term “methylation moiety” is used herein according to its plain ordinary meaning and refers to a methyl group. In embodiments, the methylation moiety is attached to an arginine, a lysine, a histidine, a glutamate, an asparagine, or a cysteine of a peptide.

The term “acetylation moiety” is used herein according to its plain ordinary meaning and refers to an acetyl group. In embodiments, the acetylation moiety is attached to a lysine, a serine, an alanine, a glycine, a threonine, or a valine of a peptide.

The term “sulfonation moiety” is used herein according to its plain ordinary meaning and refers to a sulfonic acid (—SO₂OH) group.

The term “amidation moiety” is used herein according to its plain ordinary meaning and refers to an amide group.

The term “esterification moiety” is used herein according to its plain ordinary meaning and refers to an ester group.

The term “N-terminus peptide cap moiety” is used herein according to its plain ordinary meaning and refers to a modification to the N-terminal amino acid of a peptide. In embodiments, the N-terminus peptide cap moiety prevents N-terminal degradation. In embodiments, the N-terminus peptide cap moiety includes N-terminal pyroglutamylation. In embodiments, the N-terminus peptide cap moiety includes N-terminal acetylation. In embodiments, the N-terminal acetylation mimics natural proteins. In embodiments, the N-terminal acetylation prevent N-terminal degradation.

The term “C-terminus peptide cap moiety” is used herein according to its plain ordinary meaning and refers to a modification to the C-terminal amino acid of a peptide. In embodiments, the C-terminus peptide cap moiety prevents enzymatic digestion or increases cell permeability. In embodiments, the C-terminus peptide cap moiety prevents enzymatic digestion. In embodiments, the C-terminus peptide cap moiety increases cell permeability. In embodiments, the C-terminus peptide cap moiety includes C-terminal amidation or C-terminal esterification. In embodiments, the C-terminus peptide cap moiety includes C-terminal amidation. In embodiments, the C-terminal amidation prevents enzymatic digestion. In embodiments, the C-terminal amidation is more resistant to enzymatic digestion. In embodiments, the C-terminus peptide cap moiety includes C-terminal esterification. In embodiments, the C-terminal esterification increases cell permeability.

The term “cell-penetrating peptide” is used herein according to its plain ordinary meaning and refers to an amino acid sequence that is able to pass through a tissue or a cell membrane. In embodiments, the cell-penetrating peptide is a protein transduction domain (PTD). The terms cell-penetrating peptide (CPP) and protein transduction domain (PTD) are used interchangeably herein. In embodiments, the cell-penetrating peptide is used to transport biologically active conjugates into a tissue or a cell. In embodiments, the cell-penetrating peptide is attached to the biologically active conjugate via a linker. In embodiments, the cell-penetrating peptide is attached to the biologically active conjugate via a peptide linker. In embodiments, the biologically active conjugates are peptides. In embodiments, the biologically active conjugate is a peptide provided herein including embodiments thereof. In embodiments, the peptide includes, or is, the amino acid sequence of one of SEQ ID NOs: 1-2327. In embodiments, the cell-penetrating peptide is a TAT peptide, a penetratin peptide, a buforin II peptide, a transportan peptide, a model amphipathic peptide (MAP), a K-FGF peptide, a Ku70 peptide, a prion peptide, a pVEC peptide, a Pep-1 peptide, a SynB1 peptide, a Pep-7 peptide, an HN-1 peptide, a maurocalcine peptide, a CADY peptide, or a p28 peptide. In embodiments, the cell-penetrating peptide includes the amino acid sequence of one of SEQ ID NO:2328, SEQ ID NO:2329, SEQ ID NO:2330, SEQ ID NO: 2331, SEQ ID NO: 2332, SEQ ID NO: 2333, SEQ ID NO:2334, SEQ ID NO:2335, SEQ ID NO: 2336, SEQ ID NO: 2337, SEQ ID NO: 2338, SEQ ID NO:2339, SEQ ID NO:2340, SEQ ID NO:2341, SEQ ID NO:2342, SEQ ID NO:2343, SEQ ID NO:2344, SEQ ID NO: 2345, SEQ ID NO:2346, SEQ ID NO:2347, or SEQ ID NO:2348. Cell-penetrating peptides are well known in the art (Guidotti et al., Trends Pharmacol Sci, 2017, 38 (4), pp. 406-24; Patel et al., Sci Rep, 2019, 9 (1), pp. 6298; Snyder and Dowdy, 2005, Expert Opin Drug Deliv 2, pp. 43-51; Joliot et al., Nature Cell Biology, 2004, 6 (3): 189-196; Bottens & Yamada, Cancers (Basel), 2022 November; 14 (22): 5546, each of which is incorporated herein in their entirety and for all purposes).

The terms “plasmid”, “vector” or “expression vector” refer to a nucleic acid molecule that encodes for genes, regulatory elements necessary for the expression of genes, proteins, and/or recombinant proteins (e.g., biosensors). Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide specifically reactive with a target peptide. Any appropriate method known in the art for conjugating a peptide to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

As used herein, the term “detectable marker” or “selectable marker” can refer to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection can be accomplished by optical methods such as diffraction, surface plasmon resonance, surface variation, the contact angle change or physical methods such as atomic force spectroscopy, tunnel effect, or radioactive molecules such as 32 P, 35 S or 125 I.

As used herein, the term “domain” can refer to a particular region of a protein or polypeptide, which can be associated with a particular function. For example, “a domain which binds to a cognate” can refer to the domain of a protein that binds one or more receptors or other protein moieties and (i) block the biological effect of a molecule that typically binds to the same receptor or protein or modulate the effect (i.e., increase or decrease) the biological activity of the naturally occurring binding partner of the protein or receptor.

“Contacting” is used in accordance with its plain ordinary meaning and refers to the process of allowing at least two distinct species (e.g. antibodies and antigens) to become sufficiently proximal to react, interact, or physically touch. It should be appreciated, however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a biomolecule-inhibitor interaction means negatively affecting (e.g., decreasing) the activity or function of the biomolecule (e.g. protein kinase, second messenger molecule, or GTPase) relative to the activity or function of the biomolecule in the absence of the inhibitor. In embodiments inhibition means negatively affecting (e.g., decreasing) the concentration or levels of the biomolecule relative to the concentration or level of the biomolecule in the absence of the inhibitor. In embodiments inhibition refers to reduction of a disease or symptoms of disease. In embodiments, inhibition refers to a reduction in the activity of the biomolecule. Thus, inhibition includes, at least in part, partially or totally blocking stimulation, decreasing, preventing, or delaying activation, or inactivating, desensitizing, or down-regulating signal transduction or enzymatic activity or the amount of the biomolecule. In embodiments, inhibition refers to a reduction of activity of a biomolecule resulting from a direct interaction (e.g., an inhibitor binds to a biomolecule). In embodiments, inhibition refers to a reduction of activity of a biomolecule from an indirect interaction (e.g., an inhibitor binds to a protein that activates a biomolecule, thereby preventing target protein activation).

Thus, the terms “inhibitor,” “repressor” or “antagonist” or “downregulator” interchangeably refer to a substance capable of detectably decreasing the expression or activity of a given gene or biomolecule (e.g., protein kinase, second messenger molecule, or GTPase). The antagonist can decrease the biomolecule expression or activity 10%, 20%, 30%, 40%, 50%, 60%, 70%, 80%, 90% or more in comparison to a control in the absence of the antagonist. In certain instances, biomolecule expression or activity is 1.5-fold, 2-fold, 3-fold, 4-fold, 5-fold, 10-fold or lower than the expression or activity in the absence of the antagonist.

As used herein the term “dominant-negative activity” can refer to the ability of a peptide or polypeptide of the disclosure to act as an inhibitor by binding to the wild type protein from which it is derived (i.e., from which it shares identity) or by titrating an essential ligand that binds with the protein from which the peptide is derived.

As used herein a “drug-like peptide” can refer to a polymer of amino acids comprising amide bonds that form a peptide, which does not occur in nature and which can be delivered to a cell, tissue or subject such that the biological effect results in a treatment of a disease, disorder infection or inhibits the progression of a disease, disorder or infection or which can prevent getting the disease, disorder or infection. In one embodiment, a drug-like peptide is a peptide having the sequence of any one of the sequences set forth in SEQ ID NOs: 1-2327. In another embodiment, a drug-like peptide is a peptide that is at least 95%, 96%, 97%, 98%, or 99% identical to a peptide having the sequence of any of the sequences set forth in SEQ ID NOs: 1-2327. In still another embodiment, a drug-like peptide has the sequence of any of the sequences set forth in SEQ ID NOs: 1-2327, wherein one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40) of the amino acids is substitute with a non-naturally occurring or D-amino acid and which has the same biological activity (not necessarily to the same degree) as a peptide of the same sequence having all L-amino acids. In embodiments, a drug-like peptide can be formulated for suitable routes of delivery as a pharmaceutical composition and/or linked to a second domain having a similar or different biological activity. In embodiments, a drug-like peptide can be fused to a peptide that functions to assist in delivery and/or uptake by a cell (e.g., a protein transduction domain).

The terms “equivalent” or “biological equivalent” are used interchangeably when referring to a particular molecule, biological, or cellular material and intend those having minimal homology while still maintaining desired structure or functionality.

The term “expression” includes any step involved in the production of the biomolecule including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

An “anticancer agent” as used herein refers to a molecule (e.g. compound, peptide, protein, nucleic acid) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors.

“Anti-cancer agent” and “anticancer agent” are used in accordance with their plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), histone deacetylase inhibitors (e.g., Vorinostat, Romidepsin, CI-994, Belinostat, Panobinostat, Givinostat, Entinostat, Mocetinostat, SRT501, CUDC-101, JNJ-26481585, PCI24781, etc.), PI3K inhibitors (e.g., dactolisib, omipalisib, voxtalisib, apitolisib, bimiralisib, serabelisib, tenalisib, piralalisib, taselisib, rigosertib, gedatolisib, idelalisib, copanlisib, duvelisib, umbralisib, alpelisib, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to 111 In, 90Y, or 1311, etc.), immune checkpoint inhibitors (e.g., CTLA4 blockade, PD-1 inhibitors, PD-L1 inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, adavosertib, or the like.

The term “associated” or “associated with” in the context of a substance or substance activity or function associated with a disease (e.g. a protein associated disease, a cancer) means that the disease (e.g. cancer) is caused by (in whole or in part), or a symptom of the disease is caused by (in whole or in part) the substance or substance activity or function. As used herein, what is described as being associated with a disease, if a causative agent, could be a target for treatment of the disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Peptide Compositions

Provided herein are, inter alia, peptides which can be used as therapeutics. The peptides provided herein include, for example, an amino acid sequence that can disrupt or inhibit protein-protein interactions or protein-nucleic acid interactions in a cell. The disruption or inhibition of these intracellular interactions can be used as a therapeutic in a disease or disorder (e.g., cancer) in a subject in need thereof. Thus, in an aspect is provided a peptide including an amino acid sequence having at least 80% sequence identity to the amino acid sequence of one of SEQ ID NOs: 1-2327.

In embodiments, the peptide includes an unnatural amino acid or a stabilization moiety. In embodiments, the peptide includes an unnatural amino acid. In embodiments, the peptide includes a stabilization moiety.

In embodiments, the peptide includes an amino acid sequence having at least 81% sequence identity to the amino acid sequence of one of SEQ ID NOs: 1-2327. In embodiments, the peptide includes an amino acid sequence having at least 82% sequence identity to the amino acid sequence of one of SEQ ID NOs: 1-2327. In embodiments, the peptide includes an amino acid sequence having at least 83% sequence identity to the amino acid sequence of one of SEQ ID NOs: 1-2327. In embodiments, the peptide includes an amino acid sequence having at least 84% sequence identity to the amino acid sequence of one of SEQ ID NOs: 1-2327. In embodiments, the peptide includes an amino acid sequence having at least 85% sequence identity to the amino acid sequence of one of SEQ ID NOs: 1-2327. In embodiments, the peptide includes an amino acid sequence having at least 86% sequence identity to the amino acid sequence of one of SEQ ID NOs: 1-2327. In embodiments, the peptide includes an amino acid sequence having at least 87% sequence identity to the amino acid sequence of one of SEQ ID NOs: 1-2327. In embodiments, the peptide includes an amino acid sequence having at least 88% sequence identity to the amino acid sequence of one of SEQ ID NOs: 1-2327. In embodiments, the peptide includes an amino acid sequence having at least 89% sequence identity to the amino acid sequence of one of SEQ ID NOs: 1-2327. In embodiments, the peptide includes an amino acid sequence having at least 90% sequence identity to the amino acid sequence of one of SEQ ID NOs: 1-2327. In embodiments, the peptide includes an amino acid sequence having at least 91% sequence identity to the amino acid sequence of one of SEQ ID NOs: 1-2327. In embodiments, the peptide includes an amino acid sequence having at least 92% sequence identity to the amino acid sequence of one of SEQ ID NOs: 1-2327. In embodiments, the peptide includes an amino acid sequence having at least 93% sequence identity to the amino acid sequence of one of SEQ ID NOs: 1-2327. In embodiments, the peptide includes an amino acid sequence having at least 94% sequence identity to the amino acid sequence of one of SEQ ID NOs: 1-2327. In embodiments, the peptide includes an amino acid sequence having at least 95% sequence identity to the amino acid sequence of one of SEQ ID NOs: 1-2327. In embodiments, the peptide includes an amino acid sequence having at least 96% sequence identity to the amino acid sequence of one of SEQ ID NOs: 1-2327. In embodiments, the peptide includes an amino acid sequence having at least 97% sequence identity to the amino acid sequence of one of SEQ ID NOs: 1-2327. In embodiments, the peptide includes an amino acid sequence having at least 98% sequence identity to the amino acid sequence of one of SEQ ID NOs: 1-2327. In embodiments, the peptide includes an amino acid sequence having at least 99% sequence identity to the amino acid sequence of one of SEQ ID NOs: 1-2327. In embodiments, the peptide includes an amino acid sequence having at least 100% sequence identity to the amino acid sequence of one of SEQ ID NOs: 1-2327. In embodiments, the peptide includes the amino acid sequence of one of SEQ ID NOs: 1-2327.

In embodiments, the peptide is an amino acid sequence having at least 81% sequence identity to the amino acid sequence of one of SEQ ID NOs: 1-2327. In embodiments, the peptide is an amino acid sequence having at least 82% sequence identity to the amino acid sequence of one of SEQ ID NOs: 1-2327. In embodiments, the peptide is an amino acid sequence having at least 83% sequence identity to the amino acid sequence of one of SEQ ID NOs: 1-2327. In embodiments, the peptide is an amino acid sequence having at least 84% sequence identity to the amino acid sequence of one of SEQ ID NOs: 1-2327. In embodiments, the peptide is an amino acid sequence having at least 85% sequence identity to the amino acid sequence of one of SEQ ID NOs: 1-2327. In embodiments, the peptide is an amino acid sequence having at least 86% sequence identity to the amino acid sequence of one of SEQ ID NOs: 1-2327. In embodiments, the peptide is an amino acid sequence having at least 87% sequence identity to the amino acid sequence of one of SEQ ID NOs: 1-2327. In embodiments, the peptide is an amino acid sequence having at least 88% sequence identity to the amino acid sequence of one of SEQ ID NOs: 1-2327. In embodiments, the peptide is an amino acid sequence having at least 89% sequence identity to the amino acid sequence of one of SEQ ID NOs: 1-2327. In embodiments, the peptide is an amino acid sequence having at least 90% sequence identity to the amino acid sequence of one of SEQ ID NOs: 1-2327. In embodiments, the peptide is an amino acid sequence having at least 91% sequence identity to the amino acid sequence of one of SEQ ID NOs: 1-2327. In embodiments, the peptide is an amino acid sequence having at least 92% sequence identity to the amino acid sequence of one of SEQ ID NOs: 1-2327. In embodiments, the peptide is an amino acid sequence having at least 93% sequence identity to the amino acid sequence of one of SEQ ID NOs: 1-2327. In embodiments, the peptide is an amino acid sequence having at least 94% sequence identity to the amino acid sequence of one of SEQ ID NOs: 1-2327. In embodiments, the peptide is an amino acid sequence having at least 95% sequence identity to the amino acid sequence of one of SEQ ID NOs: 1-2327. In embodiments, the peptide is an amino acid sequence having at least 96% sequence identity to the amino acid sequence of one of SEQ ID NOs: 1-2327. In embodiments, the peptide is an amino acid sequence having at least 97% sequence identity to the amino acid sequence of one of SEQ ID NOs: 1-2327. In embodiments, the peptide is an amino acid sequence having at least 98% sequence identity to the amino acid sequence of one of SEQ ID NOs: 1-2327. In embodiments, the peptide is an amino acid sequence having at least 99% sequence identity to the amino acid sequence of one of SEQ ID NOs: 1-2327. In embodiments, the peptide is an amino acid sequence having at least 100% sequence identity to the amino acid sequence of one of SEQ ID NOs: 1-2327. In embodiments, the peptide is the amino acid sequence of one of SEQ ID NOs: 1-2327.

In embodiments, the peptide includes, or is, the amino acid sequence of SEQ ID NO: 1. In embodiments, the peptide includes, or is, the amino acid sequence of SEQ ID NO:2. In embodiments, the peptide includes, or is, the amino acid sequence of SEQ ID NO:3. In embodiments, the peptide includes, or is, the amino acid sequence of SEQ ID NO:4. In embodiments, the peptide includes, or is, the amino acid sequence of SEQ ID NO:5. In embodiments, the peptide includes, or is, the amino acid sequence of SEQ ID NO:6. In embodiments, the peptide includes, or is, the amino acid sequence of SEQ ID NO:7. In embodiments, the peptide includes, or is, the amino acid sequence of SEQ ID NO:8. In embodiments, the peptide includes, or is, the amino acid sequence of SEQ ID NO:9. In embodiments, the peptide includes, or is, the amino acid sequence of SEQ ID NO:10. In embodiments, the peptide includes, or is, the amino acid sequence of SEQ ID NO:11. In embodiments, the peptide includes, or is, the amino acid sequence of SEQ ID NO: 12. In embodiments, the peptide includes, or is, the amino acid sequence of SEQ ID NO: 13. In embodiments, the peptide includes, or is, the amino acid sequence of SEQ ID NO: 14. In embodiments, the peptide includes, or is, the amino acid sequence of SEQ ID NO: 15. In embodiments, the peptide includes, or is, the amino acid sequence of SEQ ID NO:16. In embodiments, the peptide includes, or is, the amino acid sequence of SEQ ID NO: 17. In embodiments, the peptide includes, or is, the amino acid sequence of SEQ ID NO:18. In embodiments, the peptide includes, or is, the amino acid sequence of SEQ ID NO:19. In embodiments, the peptide includes, or is, the amino acid sequence of SEQ ID NO:20. In embodiments, the peptide includes, or is, the amino acid sequence of SEQ ID NO:21. In embodiments, the peptide includes, or is, the amino acid sequence of SEQ ID NO:22. In embodiments, the peptide includes, or is, the amino acid sequence of SEQ ID NO:23. In embodiments, the peptide includes, or is, the amino acid sequence of SEQ ID NO:24. In embodiments, the peptide includes, or is, the amino acid sequence of SEQ ID NO:25.

In embodiments, the peptide includes, or is, the amino acid sequence of SEQ ID NO: 26. In embodiments, the peptide includes, or is, the amino acid sequence of SEQ ID NO: 27. In embodiments, the peptide includes, or is, the amino acid sequence of SEQ ID NO: 28. In embodiments, the peptide includes, or is, the amino acid sequence of SEQ ID NO: 29. In embodiments, the peptide includes, or is, the amino acid sequence of SEQ ID NO: 30. In embodiments, the peptide includes, or is, the amino acid sequence of SEQ ID NO: 31. In embodiments, the peptide includes, or is, the amino acid sequence of SEQ ID NO: 32. In embodiments, the peptide includes, or is, the amino acid sequence of SEQ ID NO: 33. In embodiments, the peptide includes, or is, the amino acid sequence of SEQ ID NO: 34. In embodiments, the peptide includes, or is, the amino acid sequence of SEQ ID NO: 35. In embodiments, the peptide includes, or is, the amino acid sequence of SEQ ID NO: 36. In embodiments, the peptide includes, or is, the amino acid sequence of SEQ ID NO: 37. In embodiments, the peptide includes, or is, the amino acid sequence of SEQ ID NO: 38. In embodiments, the peptide includes, or is, the amino acid sequence of SEQ ID NO: 39. In embodiments, the peptide includes, or is, the amino acid sequence of SEQ ID NO: 40. In embodiments, the peptide includes, or is, the amino acid sequence of SEQ ID NO: 41. In embodiments, the peptide includes, or is, the amino acid sequence of SEQ ID NO: 42. In embodiments, the peptide includes, or is, the amino acid sequence of SEQ ID NO: 43. In embodiments, the peptide includes, or is, the amino acid sequence of SEQ ID NO: 44. In embodiments, the peptide includes, or is, the amino acid sequence of SEQ ID NO: 45. In embodiments, the peptide includes, or is, the amino acid sequence of SEQ ID NO: 46. In embodiments, the peptide includes, or is, the amino acid sequence of SEQ ID NO: 47. In embodiments, the peptide includes, or is, the amino acid sequence of SEQ ID NO: 48. In embodiments, the peptide includes, or is, the amino acid sequence of SEQ ID NO: 49. In embodiments, the peptide includes, or is, the amino acid sequence of SEQ ID NO: 50.

In embodiments, the peptide includes, or is, the amino acid sequence of SEQ ID NO: 51. In embodiments, the peptide includes, or is, the amino acid sequence of SEQ ID NO: 52. In embodiments, the peptide includes, or is, the amino acid sequence of SEQ ID NO: 53. In embodiments, the peptide includes, or is, the amino acid sequence of SEQ ID NO: 54. In embodiments, the peptide includes, or is, the amino acid sequence of SEQ ID NO: 55. In embodiments, the peptide includes, or is, the amino acid sequence of SEQ ID NO: 56. In embodiments, the peptide includes, or is, the amino acid sequence of SEQ ID NO: 57. In embodiments, the peptide includes, or is, the amino acid sequence of SEQ ID NO: 58. In embodiments, the peptide includes, or is, the amino acid sequence of SEQ ID NO: 59. In embodiments, the peptide includes, or is, the amino acid sequence of SEQ ID NO: 60. In embodiments, the peptide includes, or is, the amino acid sequence of SEQ ID NO: 61. In embodiments, the peptide includes, or is, the amino acid sequence of SEQ ID NO: 62. In embodiments, the peptide includes, or is, the amino acid sequence of SEQ ID NO: 63. In embodiments, the peptide includes, or is, the amino acid sequence of SEQ ID NO: 64. In embodiments, the peptide includes, or is, the amino acid sequence of SEQ ID NO: 65. In embodiments, the peptide includes, or is, the amino acid sequence of SEQ ID NO: 66. In embodiments, the peptide includes, or is, the amino acid sequence of SEQ ID NO: 67. In embodiments, the peptide includes, or is, the amino acid sequence of SEQ ID NO: 68. In embodiments, the peptide includes, or is, the amino acid sequence of SEQ ID NO: 69. In embodiments, the peptide includes, or is, the amino acid sequence of SEQ ID NO: 70. In embodiments, the peptide includes, or is, the amino acid sequence of SEQ ID NO: 71. In embodiments, the peptide includes, or is, the amino acid sequence of SEQ ID NO: 72. In embodiments, the peptide includes, or is, the amino acid sequence of SEQ ID NO: 73. In embodiments, the peptide includes, or is, the amino acid sequence of SEQ ID NO: 74. In embodiments, the peptide includes, or is, the amino acid sequence of SEQ ID NO: 75.

In embodiments, the peptide includes, or is, the amino acid sequence of SEQ ID NO: 76. In embodiments, the peptide includes, or is, the amino acid sequence of SEQ ID NO: 77. In embodiments, the peptide includes, or is, the amino acid sequence of SEQ ID NO: 78. In embodiments, the peptide includes, or is, the amino acid sequence of SEQ ID NO: 79. In embodiments, the peptide includes, or is, the amino acid sequence of SEQ ID NO: 80. In embodiments, the peptide includes, or is, the amino acid sequence of SEQ ID NO: 81. In embodiments, the peptide includes, or is, the amino acid sequence of SEQ ID NO: 82. In embodiments, the peptide includes, or is, the amino acid sequence of SEQ ID NO: 83. In embodiments, the peptide includes, or is, the amino acid sequence of SEQ ID NO: 84. In embodiments, the peptide includes, or is, the amino acid sequence of SEQ ID NO: 85. In embodiments, the peptide includes, or is, the amino acid sequence of SEQ ID NO: 86. In embodiments, the peptide includes, or is, the amino acid sequence of SEQ ID NO: 87. In embodiments, the peptide includes, or is, the amino acid sequence of SEQ ID NO: 88. In embodiments, the peptide includes, or is, the amino acid sequence of SEQ ID NO: 89. In embodiments, the peptide includes, or is, the amino acid sequence of SEQ ID NO: 90. In embodiments, the peptide includes, or is, the amino acid sequence of SEQ ID NO: 91. In embodiments, the peptide includes, or is, the amino acid sequence of SEQ ID NO: 92. In embodiments, the peptide includes, or is, the amino acid sequence of SEQ ID NO: 93. In embodiments, the peptide includes, or is, the amino acid sequence of SEQ ID NO: 94. In embodiments, the peptide includes, or is, the amino acid sequence of SEQ ID NO: 95. In embodiments, the peptide includes, or is, the amino acid sequence of SEQ ID NO: 96. In embodiments, the peptide includes, or is, the amino acid sequence of SEQ ID NO: 97. In embodiments, the peptide includes, or is, the amino acid sequence of SEQ ID NO: 98. In embodiments, the peptide includes, or is, the amino acid sequence of SEQ ID NO: 99. In embodiments, the peptide includes, or is, the amino acid sequence of SEQ ID NO: 100.

In embodiments, the peptide includes, or is, the amino acid sequence of SEQ ID NO: 101. In embodiments, the peptide includes, or is, the amino acid sequence of SEQ ID NO: 102. In embodiments, the peptide includes, or is, the amino acid sequence of SEQ ID NO: 103. In embodiments, the peptide includes, or is, the amino acid sequence of SEQ ID NO: 104. In embodiments, the peptide includes, or is, the amino acid sequence of SEQ ID NO: 105. In embodiments, the peptide includes, or is, the amino acid sequence of SEQ ID NO: 106. In embodiments, the peptide includes, or is, the amino acid sequence of SEQ ID NO: 107. In embodiments, the peptide includes, or is, the amino acid sequence of SEQ ID NO: 108. In embodiments, the peptide includes, or is, the amino acid sequence of SEQ ID NO: 109. In embodiments, the peptide includes, or is, the amino acid sequence of SEQ ID NO: 110. In embodiments, the peptide includes, or is, the amino acid sequence of SEQ ID NO: 111. In embodiments, the peptide includes, or is, the amino acid sequence of SEQ ID NO: 112. In embodiments, the peptide includes, or is, the amino acid sequence of SEQ ID NO: 113. In embodiments, the peptide includes, or is, the amino acid sequence of SEQ ID NO: 114. In embodiments, the peptide includes, or is, the amino acid sequence of SEQ ID NO: 115. In embodiments, the peptide includes, or is, the amino acid sequence of SEQ ID NO: 116. In embodiments, the peptide includes, or is, the amino acid sequence of SEQ ID NO: 117. In embodiments, the peptide includes, or is, the amino acid sequence of SEQ ID NO: 118. In embodiments, the peptide includes, or is, the amino acid sequence of SEQ ID NO: 119. In embodiments, the peptide includes, or is, the amino acid sequence of SEQ ID NO: 120. In embodiments, the peptide includes, or is, the amino acid sequence of SEQ ID NO: 121. In embodiments, the peptide includes, or is, the amino acid sequence of SEQ ID NO: 122. In embodiments, the peptide includes, or is, the amino acid sequence of SEQ ID NO: 123. In embodiments, the peptide includes, or is, the amino acid sequence of SEQ ID NO: 124. In embodiments, the peptide includes, or is, the amino acid sequence of SEQ ID NO: 125.

In embodiments, the peptide includes, or is, the amino acid sequence of SEQ ID NO: 126. In embodiments, the peptide includes, or is, the amino acid sequence of SEQ ID NO: 127. In embodiments, the peptide includes, or is, the amino acid sequence of SEQ ID NO: 128. In embodiments, the peptide includes, or is, the amino acid sequence of SEQ ID NO: 129. In embodiments, the peptide includes, or is, the amino acid sequence of SEQ ID NO: 130. In embodiments, the peptide includes, or is, the amino acid sequence of SEQ ID NO: 131. In embodiments, the peptide includes, or is, the amino acid sequence of SEQ ID NO: 132. In embodiments, the peptide includes, or is, the amino acid sequence of SEQ ID NO: 133. In embodiments, the peptide includes, or is, the amino acid sequence of SEQ ID NO: 134. In embodiments, the peptide includes, or is, the amino acid sequence of SEQ ID NO: 135. In embodiments, the peptide includes, or is, the amino acid sequence of SEQ ID NO: 136. In embodiments, the peptide includes, or is, the amino acid sequence of SEQ ID NO: 137. In embodiments, the peptide includes, or is, the amino acid sequence of SEQ ID NO: 138. In embodiments, the peptide includes, or is, the amino acid sequence of SEQ ID NO: 139. In embodiments, the peptide includes, or is, the amino acid sequence of SEQ ID NO: 140. In embodiments, the peptide includes, or is, the amino acid sequence of SEQ ID NO: 141. In embodiments, the peptide includes, or is, the amino acid sequence of SEQ ID NO: 142. In embodiments, the peptide includes, or is, the amino acid sequence of SEQ ID NO: 143. In embodiments, the peptide includes, or is, the amino acid sequence of SEQ ID NO: 144. In embodiments, the peptide includes, or is, the amino acid sequence of SEQ ID NO: 145. In embodiments, the peptide includes, or is, the amino acid sequence of SEQ ID NO: 146. In embodiments, the peptide includes, or is, the amino acid sequence of SEQ ID NO: 147. In embodiments, the peptide includes, or is, the amino acid sequence of SEQ ID NO: 148. In embodiments, the peptide includes, or is, the amino acid sequence of SEQ ID NO: 149. In embodiments, the peptide includes, or is, the amino acid sequence of SEQ ID NO: 150.

In embodiments, the peptide includes, or is, the amino acid sequence of SEQ ID NO: 151. In embodiments, the peptide includes, or is, the amino acid sequence of SEQ ID NO: 152. In embodiments, the peptide includes, or is, the amino acid sequence of SEQ ID NO: 153. In embodiments, the peptide includes, or is, the amino acid sequence of SEQ ID NO: 154. In embodiments, the peptide includes, or is, the amino acid sequence of SEQ ID NO: 155. In embodiments, the peptide includes, or is, the amino acid sequence of SEQ ID NO: 156. In embodiments, the peptide includes, or is, the amino acid sequence of SEQ ID NO: 157. In embodiments, the peptide includes, or is, the amino acid sequence of SEQ ID NO: 158. In embodiments, the peptide includes, or is, the amino acid sequence of SEQ ID NO: 159. In embodiments, the peptide includes, or is, the amino acid sequence of SEQ ID NO: 160. In embodiments, the peptide includes, or is, the amino acid sequence of SEQ ID NO: 161. In embodiments, the peptide includes, or is, the amino acid sequence of SEQ ID NO: 162. In embodiments, the peptide includes, or is, the amino acid sequence of SEQ ID NO: 163. In embodiments, the peptide includes, or is, the amino acid sequence of SEQ ID NO: 164. In embodiments, the peptide includes, or is, the amino acid sequence of SEQ ID NO: 165. In embodiments, the peptide includes, or is, the amino acid sequence of SEQ ID NO: 166. In embodiments, the peptide includes, or is, the amino acid sequence of SEQ ID NO: 167. In embodiments, the peptide includes, or is, the amino acid sequence of SEQ ID NO: 168. In embodiments, the peptide includes, or is, the amino acid sequence of SEQ ID NO: 169. In embodiments, the peptide includes, or is, the amino acid sequence of SEQ ID NO: 170. In embodiments, the peptide includes, or is, the amino acid sequence of SEQ ID NO: 171. In embodiments, the peptide includes, or is, the amino acid sequence of SEQ ID NO: 172. In embodiments, the peptide includes, or is, the amino acid sequence of SEQ ID NO: 173. In embodiments, the peptide includes, or is, the amino acid sequence of SEQ ID NO: 174.

In embodiments, the peptide includes, or is, the amino acid sequence of SEQ ID NO: 175. In embodiments, the peptide includes, or is, the amino acid sequence of SEQ ID NO: 176. In embodiments, the peptide includes, or is, the amino acid sequence of SEQ ID NO: 177. In embodiments, the peptide includes, or is, the amino acid sequence of SEQ ID NO: 178. In embodiments, the peptide includes, or is, the amino acid sequence of SEQ ID NO: 179. In embodiments, the peptide includes, or is, the amino acid sequence of SEQ ID NO: 180. In embodiments, the peptide includes, or is, the amino acid sequence of SEQ ID NO: 181. In embodiments, the peptide includes, or is, the amino acid sequence of SEQ ID NO: 182. In embodiments, the peptide includes, or is, the amino acid sequence of SEQ ID NO: 183. In embodiments, the peptide includes, or is, the amino acid sequence of SEQ ID NO: 184. In embodiments, the peptide includes, or is, the amino acid sequence of SEQ ID NO: 185. In embodiments, the peptide includes, or is, the amino acid sequence of SEQ ID NO: 186. In embodiments, the peptide includes, or is, the amino acid sequence of SEQ ID NO: 187. In embodiments, the peptide includes, or is, the amino acid sequence of SEQ ID NO: 188. In embodiments, the peptide includes, or is, the amino acid sequence of SEQ ID NO: 189. In embodiments, the peptide includes, or is, the amino acid sequence of SEQ ID NO: 190. In embodiments, the peptide includes, or is, the amino acid sequence of SEQ ID NO: 191. In embodiments, the peptide includes, or is, the amino acid sequence of SEQ ID NO: 192. In embodiments, the peptide includes, or is, the amino acid sequence of SEQ ID NO: 193. In embodiments, the peptide includes, or is, the amino acid sequence of SEQ ID NO: 194. In embodiments, the peptide includes, or is, the amino acid sequence of SEQ ID NO: 195. In embodiments, the peptide includes, or is, the amino acid sequence of SEQ ID NO: 196. In embodiments, the peptide includes, or is, the amino acid sequence of SEQ ID NO: 197. In embodiments, the peptide includes, or is, the amino acid sequence of SEQ ID NO: 198. In embodiments, the peptide includes, or is, the amino acid sequence of SEQ ID NO: 199. In embodiments, the peptide includes, or is, the amino acid sequence of SEQ ID NO: 200.

In embodiments, the peptide includes, or is, the amino acid sequence of SEQ ID NO: 201. In embodiments, the peptide includes, or is, the amino acid sequence of SEQ ID NO: 202. In embodiments, the peptide includes, or is, the amino acid sequence of SEQ ID NO: 203. In embodiments, the peptide includes, or is, the amino acid sequence of SEQ ID NO: 204. In embodiments, the peptide includes, or is, the amino acid sequence of SEQ ID NO: 205. In embodiments, the peptide includes, or is, the amino acid sequence of SEQ ID NO: 206. In embodiments, the peptide includes, or is, the amino acid sequence of SEQ ID NO: 207. In embodiments, the peptide includes, or is, the amino acid sequence of SEQ ID NO: 208. In embodiments, the peptide includes, or is, the amino acid sequence of SEQ ID NO: 209. In embodiments, the peptide includes, or is, the amino acid sequence of SEQ ID NO: 210. In embodiments, the peptide includes, or is, the amino acid sequence of SEQ ID NO: 211. In embodiments, the peptide includes, or is, the amino acid sequence of SEQ ID NO: 212. In embodiments, the peptide includes, or is, the amino acid sequence of SEQ ID NO: 213. In embodiments, the peptide includes, or is, the amino acid sequence of SEQ ID NO: 214. In embodiments, the peptide includes, or is, the amino acid sequence of SEQ ID NO: 215. In embodiments, the peptide includes, or is, the amino acid sequence of SEQ ID NO: 216. In embodiments, the peptide includes, or is, the amino acid sequence of SEQ ID NO: 217. In embodiments, the peptide includes, or is, the amino acid sequence of SEQ ID NO: 218. In embodiments, the peptide includes, or is, the amino acid sequence of SEQ ID NO: 219. In embodiments, the peptide includes, or is, the amino acid sequence of SEQ ID NO: 220. In embodiments, the peptide includes, or is, the amino acid sequence of SEQ ID NO: 221. In embodiments, the peptide includes, or is, the amino acid sequence of SEQ ID NO: 222. In embodiments, the peptide includes, or is, the amino acid sequence of SEQ ID NO: 223. In embodiments, the peptide includes, or is, the amino acid sequence of SEQ ID NO: 224. In embodiments, the peptide includes, or is, the amino acid sequence of SEQ ID NO: 225.

In embodiments, the peptide includes, or is, the amino acid sequence of SEQ ID NO: 226. In embodiments, the peptide includes, or is, the amino acid sequence of SEQ ID NO: 227. In embodiments, the peptide includes, or is, the amino acid sequence of SEQ ID NO: 228. In embodiments, the peptide includes, or is, the amino acid sequence of SEQ ID NO: 229. In embodiments, the peptide includes, or is, the amino acid sequence of SEQ ID NO: 230. In embodiments, the peptide includes, or is, the amino acid sequence of SEQ ID NO: 231. In embodiments, the peptide includes, or is, the amino acid sequence of SEQ ID NO: 232. In embodiments, the peptide includes, or is, the amino acid sequence of SEQ ID NO: 233. In embodiments, the peptide includes, or is, the amino acid sequence of SEQ ID NO: 234. In embodiments, the peptide includes, or is, the amino acid sequence of SEQ ID NO: 235. In embodiments, the peptide includes, or is, the amino acid sequence of SEQ ID NO: 236. In embodiments, the peptide includes, or is, the amino acid sequence of SEQ ID NO: 237. In embodiments, the peptide includes, or is, the amino acid sequence of SEQ ID NO: 238. In embodiments, the peptide includes, or is, the amino acid sequence of SEQ ID NO: 239. In embodiments, the peptide includes, or is, the amino acid sequence of SEQ ID NO: 240. In embodiments, the peptide includes, or is, the amino acid sequence of SEQ ID NO: 241. In embodiments, the peptide includes, or is, the amino acid sequence of SEQ ID NO: 242. In embodiments, the peptide includes, or is, the amino acid sequence of SEQ ID NO: 243. In embodiments, the peptide includes, or is, the amino acid sequence of SEQ ID NO: 244. In embodiments, the peptide includes, or is, the amino acid sequence of SEQ ID NO: 245. In embodiments, the peptide includes, or is, the amino acid sequence of SEQ ID NO: 246. In embodiments, the peptide includes, or is, the amino acid sequence of SEQ ID NO: 247. In embodiments, the peptide includes, or is, the amino acid sequence of SEQ ID NO: 248. In embodiments, the peptide includes, or is, the amino acid sequence of SEQ ID NO: 249. In embodiments, the peptide includes, or is, the amino acid sequence of SEQ ID NO: 250.

In embodiments, the peptide includes, or is, the amino acid sequence of SEQ ID NO: 251. In embodiments, the peptide includes, or is, the amino acid sequence of SEQ ID NO: 252. In embodiments, the peptide includes, or is, the amino acid sequence of SEQ ID NO: 253. In embodiments, the peptide includes, or is, the amino acid sequence of SEQ ID NO: 254. In embodiments, the peptide includes, or is, the amino acid sequence of SEQ ID NO: 255. In embodiments, the peptide includes, or is, the amino acid sequence of SEQ ID NO: 256. In embodiments, the peptide includes, or is, the amino acid sequence of SEQ ID NO: 257. In embodiments, the peptide includes, or is, the amino acid sequence of SEQ ID NO: 258. In embodiments, the peptide includes, or is, the amino acid sequence of SEQ ID NO: 259. In embodiments, the peptide includes, or is, the amino acid sequence of SEQ ID NO: 260. In embodiments, the peptide includes, or is, the amino acid sequence of SEQ ID NO: 261. In embodiments, the peptide includes, or is, the amino acid sequence of SEQ ID NO: 262. In embodiments, the peptide includes, or is, the amino acid sequence of SEQ ID NO: 263. In embodiments, the peptide includes, or is, the amino acid sequence of SEQ ID NO: 264. In embodiments, the peptide includes, or is, the amino acid sequence of SEQ ID NO: 265. In embodiments, the peptide includes, or is, the amino acid sequence of SEQ ID NO: 266. In embodiments, the peptide includes, or is, the amino acid sequence of SEQ ID NO: 267. In embodiments, the peptide includes, or is, the amino acid sequence of SEQ ID NO: 268. In embodiments, the peptide includes, or is, the amino acid sequence of SEQ ID NO: 269. In embodiments, the peptide includes, or is, the amino acid sequence of SEQ ID NO: 270. In embodiments, the peptide includes, or is, the amino acid sequence of SEQ ID NO: 271. In embodiments, the peptide includes, or is, the amino acid sequence of SEQ ID NO: 272. In embodiments, the peptide includes, or is, the amino acid sequence of SEQ ID NO: 273. In embodiments, the peptide includes, or is, the amino acid sequence of SEQ ID NO: 274. In embodiments, the peptide includes, or is, the amino acid sequence of SEQ ID NO: 275.

In embodiments, the peptide includes, or is, the amino acid sequence of SEQ ID NO: 276. In embodiments, the peptide includes, or is, the amino acid sequence of SEQ ID NO: 277. In embodiments, the peptide includes, or is, the amino acid sequence of SEQ ID NO: 278. In embodiments, the peptide includes, or is, the amino acid sequence of SEQ ID NO: 279. In embodiments, the peptide includes, or is, the amino acid sequence of SEQ ID NO: 280. In embodiments, the peptide includes, or is, the amino acid sequence of SEQ ID NO: 281. In embodiments, the peptide includes, or is, the amino acid sequence of SEQ ID NO: 282. In embodiments, the peptide includes, or is, the amino acid sequence of SEQ ID NO: 283. In embodiments, the peptide includes, or is, the amino acid sequence of SEQ ID NO: 284. In embodiments, the peptide includes, or is, the amino acid sequence of SEQ ID NO: 285. In embodiments, the peptide includes, or is, the amino acid sequence of SEQ ID NO: 286. In embodiments, the peptide includes, or is, the amino acid sequence of SEQ ID NO: 287. In embodiments, the peptide includes, or is, the amino acid sequence of SEQ ID NO: 288. In embodiments, the peptide includes, or is, the amino acid sequence of SEQ ID NO: 289. In embodiments, the peptide includes, or is, the amino acid sequence of SEQ ID NO: 290. In embodiments, the peptide includes, or is, the amino acid sequence of SEQ ID NO: 291. In embodiments, the peptide includes, or is, the amino acid sequence of SEQ ID NO: 292. In embodiments, the peptide includes, or is, the amino acid sequence of SEQ ID NO: 293. In embodiments, the peptide includes, or is, the amino acid sequence of SEQ ID NO: 294. In embodiments, the peptide includes, or is, the amino acid sequence of SEQ ID NO: 295. In embodiments, the peptide includes, or is, the amino acid sequence of SEQ ID NO: 296. In embodiments, the peptide includes, or is, the amino acid sequence of SEQ ID NO: 297. In embodiments, the peptide includes, or is, the amino acid sequence of SEQ ID NO: 298. In embodiments, the peptide includes, or is, the amino acid sequence of SEQ ID NO: 299. In embodiments, the peptide includes, or is, the amino acid sequence of SEQ ID NO: 300.

In embodiments, the peptide includes, or is, the amino acid sequence of SEQ ID NO: 301. In embodiments, the peptide includes, or is, the amino acid sequence of SEQ ID NO: 302. In embodiments, the peptide includes, or is, the amino acid sequence of SEQ ID NO: 303. In embodiments, the peptide includes, or is, the amino acid sequence of SEQ ID NO: 304. In embodiments, the peptide includes, or is, the amino acid sequence of SEQ ID NO: 305. In embodiments, the peptide includes, or is, the amino acid sequence of SEQ ID NO: 306. In embodiments, the peptide includes, or is, the amino acid sequence of SEQ ID NO: 307. In embodiments, the peptide includes, or is, the amino acid sequence of SEQ ID NO: 308. In embodiments, the peptide includes, or is, the amino acid sequence of SEQ ID NO: 309. In embodiments, the peptide includes, or is, the amino acid sequence of SEQ ID NO: 310. In embodiments, the peptide includes, or is, the amino acid sequence of SEQ ID NO: 311. In embodiments, the peptide includes, or is, the amino acid sequence of SEQ ID NO: 312. In embodiments, the peptide includes, or is, the amino acid sequence of SEQ ID NO: 313. In embodiments, the peptide includes, or is, the amino acid sequence of SEQ ID NO: 314. In embodiments, the peptide includes, or is, the amino acid sequence of SEQ ID NO: 315. In embodiments, the peptide includes, or is, the amino acid sequence of SEQ ID NO: 316. In embodiments, the peptide includes, or is, the amino acid sequence of SEQ ID NO: 317. In embodiments, the peptide includes, or is, the amino acid sequence of SEQ ID NO: 318. In embodiments, the peptide includes, or is, the amino acid sequence of SEQ ID NO: 319. In embodiments, the peptide includes, or is, the amino acid sequence of SEQ ID NO: 320. In embodiments, the peptide includes, or is, the amino acid sequence of SEQ ID NO: 321. In embodiments, the peptide includes, or is, the amino acid sequence of SEQ ID NO: 322. In embodiments, the peptide includes, or is, the amino acid sequence of SEQ ID NO: 323. In embodiments, the peptide includes, or is, the amino acid sequence of SEQ ID NO: 324. In embodiments, the peptide includes, or is, the amino acid sequence of SEQ ID NO: 325.

In embodiments, the peptide includes, or is, the amino acid sequence of SEQ ID NO: 326. In embodiments, the peptide includes, or is, the amino acid sequence of SEQ ID NO: 327. In embodiments, the peptide includes, or is, the amino acid sequence of SEQ ID NO: 328. In embodiments, the peptide includes, or is, the amino acid sequence of SEQ ID NO: 329. In embodiments, the peptide includes, or is, the amino acid sequence of SEQ ID NO: 330. In embodiments, the peptide includes, or is, the amino acid sequence of SEQ ID NO: 331. In embodiments, the peptide includes, or is, the amino acid sequence of SEQ ID NO: 332. In embodiments, the peptide includes, or is, the amino acid sequence of SEQ ID NO: 333. In embodiments, the peptide includes, or is, the amino acid sequence of SEQ ID NO: 334. In embodiments, the peptide includes, or is, the amino acid sequence of SEQ ID NO: 335. In embodiments, the peptide includes, or is, the amino acid sequence of SEQ ID NO: 336. In embodiments, the peptide includes, or is, the amino acid sequence of SEQ ID NO: 337. In embodiments, the peptide includes, or is, the amino acid sequence of SEQ ID NO: 338. In embodiments, the peptide includes, or is, the amino acid sequence of SEQ ID NO: 339. In embodiments, the peptide includes, or is, the amino acid sequence of SEQ ID NO: 340. In embodiments, the peptide includes, or is, the amino acid sequence of SEQ ID NO: 341. In embodiments, the peptide includes, or is, the amino acid sequence of SEQ ID NO: 342. In embodiments, the peptide includes, or is, the amino acid sequence of SEQ ID NO: 343. In embodiments, the peptide includes, or is, the amino acid sequence of SEQ ID NO: 344. In embodiments, the peptide includes, or is, the amino acid sequence of SEQ ID NO: 345. In embodiments, the peptide includes, or is, the amino acid sequence of SEQ ID NO: 346. In embodiments, the peptide includes, or is, the amino acid sequence of SEQ ID NO: 347. In embodiments, the peptide includes, or is, the amino acid sequence of SEQ ID NO: 348. In embodiments, the peptide includes, or is, the amino acid sequence of SEQ ID NO: 349. In embodiments, the peptide includes, or is, the amino acid sequence of SEQ ID NO: 350.

In embodiments, the peptide includes, or is, the amino acid sequence of SEQ ID NO: 351. In embodiments, the peptide includes, or is, the amino acid sequence of SEQ ID NO: 352. In embodiments, the peptide includes, or is, the amino acid sequence of SEQ ID NO: 353. In embodiments, the peptide includes, or is, the amino acid sequence of SEQ ID NO: 354. In embodiments, the peptide includes, or is, the amino acid sequence of SEQ ID NO: 355. In embodiments, the peptide includes, or is, the amino acid sequence of SEQ ID NO: 356. In embodiments, the peptide includes, or is, the amino acid sequence of SEQ ID NO: 357. In embodiments, the peptide includes, or is, the amino acid sequence of SEQ ID NO: 358. In embodiments, the peptide includes, or is, the amino acid sequence of SEQ ID NO: 359. In embodiments, the peptide includes, or is, the amino acid sequence of SEQ ID NO: 360. In embodiments, the peptide includes, or is, the amino acid sequence of SEQ ID NO: 361. In embodiments, the peptide includes, or is, the amino acid sequence of SEQ ID NO: 362. In embodiments, the peptide includes, or is, the amino acid sequence of SEQ ID NO: 363. In embodiments, the peptide includes, or is, the amino acid sequence of SEQ ID NO: 364. In embodiments, the peptide includes, or is, the amino acid sequence of SEQ ID NO: 365. In embodiments, the peptide includes, or is, the amino acid sequence of SEQ ID NO: 366. In embodiments, the peptide includes, or is, the amino acid sequence of SEQ ID NO: 367. In embodiments, the peptide includes, or is, the amino acid sequence of SEQ ID NO: 368. In embodiments, the peptide includes, or is, the amino acid sequence of SEQ ID NO: 369. In embodiments, the peptide includes, or is, the amino acid sequence of SEQ ID NO: 370. In embodiments, the peptide includes, or is, the amino acid sequence of SEQ ID NO: 371. In embodiments, the peptide includes, or is, the amino acid sequence of SEQ ID NO: 372. In embodiments, the peptide includes, or is, the amino acid sequence of SEQ ID NO: 373. In embodiments, the peptide includes, or is, the amino acid sequence of SEQ ID NO: 374. In embodiments, the peptide includes, or is, the amino acid sequence of SEQ ID NO: 375.

In embodiments, the peptide includes, or is, the amino acid sequence of SEQ ID NO: 376. In embodiments, the peptide includes, or is, the amino acid sequence of SEQ ID NO: 377. In embodiments, the peptide includes, or is, the amino acid sequence of SEQ ID NO: 378. In embodiments, the peptide includes, or is, the amino acid sequence of SEQ ID NO: 379. In embodiments, the peptide includes, or is, the amino acid sequence of SEQ ID NO: 380. In embodiments, the peptide includes, or is, the amino acid sequence of SEQ ID NO: 381. In embodiments, the peptide includes, or is, the amino acid sequence of SEQ ID NO: 382. In embodiments, the peptide includes, or is, the amino acid sequence of SEQ ID NO: 383. In embodiments, the peptide includes, or is, the amino acid sequence of SEQ ID NO: 384. In embodiments, the peptide includes, or is, the amino acid sequence of SEQ ID NO: 385. In embodiments, the peptide includes, or is, the amino acid sequence of SEQ ID NO: 386. In embodiments, the peptide includes, or is, the amino acid sequence of SEQ ID NO: 387. In embodiments, the peptide includes, or is, the amino acid sequence of SEQ ID NO: 388. In embodiments, the peptide includes, or is, the amino acid sequence of SEQ ID NO: 389. In embodiments, the peptide includes, or is, the amino acid sequence of SEQ ID NO: 390. In embodiments, the peptide includes, or is, the amino acid sequence of SEQ ID NO: 391. In embodiments, the peptide includes, or is, the amino acid sequence of SEQ ID NO: 392. In embodiments, the peptide includes, or is, the amino acid sequence of SEQ ID NO: 393. In embodiments, the peptide includes, or is, the amino acid sequence of SEQ ID NO: 394. In embodiments, the peptide includes, or is, the amino acid sequence of SEQ ID NO: 395. In embodiments, the peptide includes, or is, the amino acid sequence of SEQ ID NO: 396. In embodiments, the peptide includes, or is, the amino acid sequence of SEQ ID NO: 397. In embodiments, the peptide includes, or is, the amino acid sequence of SEQ ID NO: 398. In embodiments, the peptide includes, or is, the amino acid sequence of SEQ ID NO: 399. In embodiments, the peptide includes, or is, the amino acid sequence of SEQ ID NO: 400.

In embodiments, the peptide includes, or is, the amino acid sequence of SEQ ID NO: 401. In embodiments, the peptide includes, or is, the amino acid sequence of SEQ ID NO: 402. In embodiments, the peptide includes, or is, the amino acid sequence of SEQ ID NO: 403. In embodiments, the peptide includes, or is, the amino acid sequence of SEQ ID NO: 404. In embodiments, the peptide includes, or is, the amino acid sequence of SEQ ID NO: 405. In embodiments, the peptide includes, or is, the amino acid sequence of SEQ ID NO: 406. In embodiments, the peptide includes, or is, the amino acid sequence of SEQ ID NO: 407. In embodiments, the peptide includes, or is, the amino acid sequence of SEQ ID NO: 408. In embodiments, the peptide includes, or is, the amino acid sequence of SEQ ID NO: 409. In embodiments, the peptide includes, or is, the amino acid sequence of SEQ ID NO: 410. In embodiments, the peptide includes, or is, the amino acid sequence of SEQ ID NO: 411. In embodiments, the peptide includes, or is, the amino acid sequence of SEQ ID NO: 412. In embodiments, the peptide includes, or is, the amino acid sequence of SEQ ID NO: 413. In embodiments, the peptide includes, or is, the amino acid sequence of SEQ ID NO: 414. In embodiments, the peptide includes, or is, the amino acid sequence of SEQ ID NO: 415. In embodiments, the peptide includes, or is, the amino acid sequence of SEQ ID NO: 416. In embodiments, the peptide includes, or is, the amino acid sequence of SEQ ID NO: 417. In embodiments, the peptide includes, or is, the amino acid sequence of SEQ ID NO: 418. In embodiments, the peptide includes, or is, the amino acid sequence of SEQ ID NO: 419. In embodiments, the peptide includes, or is, the amino acid sequence of SEQ ID NO: 420. In embodiments, the peptide includes, or is, the amino acid sequence of SEQ ID NO: 421. In embodiments, the peptide includes, or is, the amino acid sequence of SEQ ID NO: 422. In embodiments, the peptide includes, or is, the amino acid sequence of SEQ ID NO: 423. In embodiments, the peptide includes, or is, the amino acid sequence of SEQ ID NO: 424. In embodiments, the peptide includes, or is, the amino acid sequence of SEQ ID NO: 425.

In embodiments, the peptide includes, or is, the amino acid sequence of SEQ ID NO: 426. In embodiments, the peptide includes, or is, the amino acid sequence of SEQ ID NO: 427. In embodiments, the peptide includes, or is, the amino acid sequence of SEQ ID NO: 428. In embodiments, the peptide includes, or is, the amino acid sequence of SEQ ID NO: 429. In embodiments, the peptide includes, or is, the amino acid sequence of SEQ ID NO: 430. In embodiments, the peptide includes, or is, the amino acid sequence of SEQ ID NO: 431. In embodiments, the peptide includes, or is, the amino acid sequence of SEQ ID NO: 432. In embodiments, the peptide includes, or is, the amino acid sequence of SEQ ID NO: 433. In embodiments, the peptide includes, or is, the amino acid sequence of SEQ ID NO: 434. In embodiments, the peptide includes, or is, the amino acid sequence of SEQ ID NO: 435. In embodiments, the peptide includes, or is, the amino acid sequence of SEQ ID NO: 436. In embodiments, the peptide includes, or is, the amino acid sequence of SEQ ID NO: 437. In embodiments, the peptide includes, or is, the amino acid sequence of SEQ ID NO: 438. In embodiments, the peptide includes, or is, the amino acid sequence of SEQ ID NO: 439. In embodiments, the peptide includes, or is, the amino acid sequence of SEQ ID NO: 440. In embodiments, the peptide includes, or is, the amino acid sequence of SEQ ID NO: 441. In embodiments, the peptide includes, or is, the amino acid sequence of SEQ ID NO: 442. In embodiments, the peptide includes, or is, the amino acid sequence of SEQ ID NO: 443. In embodiments, the peptide includes, or is, the amino acid sequence of SEQ ID NO: 444. In embodiments, the peptide includes, or is, the amino acid sequence of SEQ ID NO: 445. In embodiments, the peptide includes, or is, the amino acid sequence of SEQ ID NO: 446. In embodiments, the peptide includes, or is, the amino acid sequence of SEQ ID NO: 447. In embodiments, the peptide includes, or is, the amino acid sequence of SEQ ID NO: 448. In embodiments, the peptide includes, or is, the amino acid sequence of SEQ ID NO: 449. In embodiments, the peptide includes, or is, the amino acid sequence of SEQ ID NO: 450.

In embodiments, the peptide includes, or is, the amino acid sequence of SEQ ID NO: 451. In embodiments, the peptide includes, or is, the amino acid sequence of SEQ ID NO: 452. In embodiments, the peptide includes, or is, the amino acid sequence of SEQ ID NO: 453. In embodiments, the peptide includes, or is, the amino acid sequence of SEQ ID NO: 454. In embodiments, the peptide includes, or is, the amino acid sequence of SEQ ID NO: 455. In embodiments, the peptide includes, or is, the amino acid sequence of SEQ ID NO: 456. In embodiments, the peptide includes, or is, the amino acid sequence of SEQ ID NO: 457. In embodiments, the peptide includes, or is, the amino acid sequence of SEQ ID NO: 458. In embodiments, the peptide includes, or is, the amino acid sequence of SEQ ID NO: 459. In embodiments, the peptide includes, or is, the amino acid sequence of SEQ ID NO: 460. In embodiments, the peptide includes, or is, the amino acid sequence of SEQ ID NO: 461. In embodiments, the peptide includes, or is, the amino acid sequence of SEQ ID NO: 462. In embodiments, the peptide includes, or is, the amino acid sequence of SEQ ID NO: 463. In embodiments, the peptide includes, or is, the amino acid sequence of SEQ ID NO: 464. In embodiments, the peptide includes, or is, the amino acid sequence of SEQ ID NO: 465. In embodiments, the peptide includes, or is, the amino acid sequence of SEQ ID NO: 466. In embodiments, the peptide includes, or is, the amino acid sequence of SEQ ID NO: 467. In embodiments, the peptide includes, or is, the amino acid sequence of SEQ ID NO: 468. In embodiments, the peptide includes, or is, the amino acid sequence of SEQ ID NO: 469. In embodiments, the peptide includes, or is, the amino acid sequence of SEQ ID NO: 470. In embodiments, the peptide includes, or is, the amino acid sequence of SEQ ID NO: 471. In embodiments, the peptide includes, or is, the amino acid sequence of SEQ ID NO: 472. In embodiments, the peptide includes, or is, the amino acid sequence of SEQ ID NO: 473. In embodiments, the peptide includes, or is, the amino acid sequence of SEQ ID NO: 474. In embodiments, the peptide includes, or is, the amino acid sequence of SEQ ID NO: 475.

In embodiments, the peptide includes, or is, the amino acid sequence of SEQ ID NO: 476. In embodiments, the peptide includes, or is, the amino acid sequence of SEQ ID NO: 477. In embodiments, the peptide includes, or is, the amino acid sequence of SEQ ID NO: 478. In embodiments, the peptide includes, or is, the amino acid sequence of SEQ ID NO: 479. In embodiments, the peptide includes, or is, the amino acid sequence of SEQ ID NO: 480. In embodiments, the peptide includes, or is, the amino acid sequence of SEQ ID NO: 481. In embodiments, the peptide includes, or is, the amino acid sequence of SEQ ID NO: 482. In embodiments, the peptide includes, or is, the amino acid sequence of SEQ ID NO: 483. In embodiments, the peptide includes, or is, the amino acid sequence of SEQ ID NO: 484. In embodiments, the peptide includes, or is, the amino acid sequence of SEQ ID NO: 485. In embodiments, the peptide includes, or is, the amino acid sequence of SEQ ID NO: 486. In embodiments, the peptide includes, or is, the amino acid sequence of SEQ ID NO: 487. In embodiments, the peptide includes, or is, the amino acid sequence of SEQ ID NO: 488. In embodiments, the peptide includes, or is, the amino acid sequence of SEQ ID NO: 489. In embodiments, the peptide includes, or is, the amino acid sequence of SEQ ID NO: 490. In embodiments, the peptide includes, or is, the amino acid sequence of SEQ ID NO: 491. In embodiments, the peptide includes, or is, the amino acid sequence of SEQ ID NO: 492. In embodiments, the peptide includes, or is, the amino acid sequence of SEQ ID NO: 493. In embodiments, the peptide includes, or is, the amino acid sequence of SEQ ID NO: 494. In embodiments, the peptide includes, or is, the amino acid sequence of SEQ ID NO: 495. In embodiments, the peptide includes, or is, the amino acid sequence of SEQ ID NO: 496. In embodiments, the peptide includes, or is, the amino acid sequence of SEQ ID NO: 497. In embodiments, the peptide includes, or is, the amino acid sequence of SEQ ID NO: 498. In embodiments, the peptide includes, or is, the amino acid sequence of SEQ ID NO: 499. In embodiments, the peptide includes, or is, the amino acid sequence of SEQ ID NO: 500.

In embodiments, the peptide includes, or is, the amino acid sequence of SEQ ID NO: 501. In embodiments, the peptide includes, or is, the amino acid sequence of SEQ ID NO: 502. In embodiments, the peptide includes, or is, the amino acid sequence of SEQ ID NO: 503. In embodiments, the peptide includes, or is, the amino acid sequence of SEQ ID NO: 504. In embodiments, the peptide includes, or is, the amino acid sequence of SEQ ID NO: 505. In embodiments, the peptide includes, or is, the amino acid sequence of SEQ ID NO: 506. In embodiments, the peptide includes, or is, the amino acid sequence of SEQ ID NO: 507. In embodiments, the peptide includes, or is, the amino acid sequence of SEQ ID NO: 508. In embodiments, the peptide includes, or is, the amino acid sequence of SEQ ID NO: 509. In embodiments, the peptide includes, or is, the amino acid sequence of SEQ ID NO: 510. In embodiments, the peptide includes, or is, the amino acid sequence of SEQ ID NO: 511. In embodiments, the peptide includes, or is, the amino acid sequence of SEQ ID NO: 512. In embodiments, the peptide includes, or is, the amino acid sequence of SEQ ID NO: 513. In embodiments, the peptide includes, or is, the amino acid sequence of SEQ ID NO: 514. In embodiments, the peptide includes, or is, the amino acid sequence of SEQ ID NO: 515. In embodiments, the peptide includes, or is, the amino acid sequence of SEQ ID NO: 516. In embodiments, the peptide includes, or is, the amino acid sequence of SEQ ID NO: 517. In embodiments, the peptide includes, or is, the amino acid sequence of SEQ ID NO: 518. In embodiments, the peptide includes, or is, the amino acid sequence of SEQ ID NO: 519. In embodiments, the peptide includes, or is, the amino acid sequence of SEQ ID NO: 520. In embodiments, the peptide includes, or is, the amino acid sequence of SEQ ID NO: 521. In embodiments, the peptide includes, or is, the amino acid sequence of SEQ ID NO: 522. In embodiments, the peptide includes, or is, the amino acid sequence of SEQ ID NO: 523. In embodiments, the peptide includes, or is, the amino acid sequence of SEQ ID NO: 524. In embodiments, the peptide includes, or is, the amino acid sequence of SEQ ID NO: 525.

In embodiments, the peptide includes, or is, the amino acid sequence of SEQ ID NO: 526. In embodiments, the peptide includes, or is, the amino acid sequence of SEQ ID NO: 527. In embodiments, the peptide includes, or is, the amino acid sequence of SEQ ID NO: 528. In embodiments, the peptide includes, or is, the amino acid sequence of SEQ ID NO: 529. In embodiments, the peptide includes, or is, the amino acid sequence of SEQ ID NO: 530. In embodiments, the peptide includes, or is, the amino acid sequence of SEQ ID NO: 531. In embodiments, the peptide includes, or is, the amino acid sequence of SEQ ID NO: 532. In embodiments, the peptide includes, or is, the amino acid sequence of SEQ ID NO: 533. In embodiments, the peptide includes, or is, the amino acid sequence of SEQ ID NO: 534. In embodiments, the peptide includes, or is, the amino acid sequence of SEQ ID NO: 535. In embodiments, the peptide includes, or is, the amino acid sequence of SEQ ID NO: 536. In embodiments, the peptide includes, or is, the amino acid sequence of SEQ ID NO: 537. In embodiments, the peptide includes, or is, the amino acid sequence of SEQ ID NO: 538. In embodiments, the peptide includes, or is, the amino acid sequence of SEQ ID NO: 539. In embodiments, the peptide includes, or is, the amino acid sequence of SEQ ID NO: 540. In embodiments, the peptide includes, or is, the amino acid sequence of SEQ ID NO: 541. In embodiments, the peptide includes, or is, the amino acid sequence of SEQ ID NO: 542. In embodiments, the peptide includes, or is, the amino acid sequence of SEQ ID NO: 543. In embodiments, the peptide includes, or is, the amino acid sequence of SEQ ID NO: 544. In embodiments, the peptide includes, or is, the amino acid sequence of SEQ ID NO: 545. In embodiments, the peptide includes, or is, the amino acid sequence of SEQ ID NO: 546. In embodiments, the peptide includes, or is, the amino acid sequence of SEQ ID NO: 547. In embodiments, the peptide includes, or is, the amino acid sequence of SEQ ID NO: 548. In embodiments, the peptide includes, or is, the amino acid sequence of SEQ ID NO: 549. In embodiments, the peptide includes, or is, the amino acid sequence of SEQ ID NO: 550.

In embodiments, the peptide includes, or is, the amino acid sequence of SEQ ID NO: 551. In embodiments, the peptide includes, or is, the amino acid sequence of SEQ ID NO: 552. In embodiments, the peptide includes, or is, the amino acid sequence of SEQ ID NO: 553. In embodiments, the peptide includes, or is, the amino acid sequence of SEQ ID NO: 554. In embodiments, the peptide includes, or is, the amino acid sequence of SEQ ID NO: 555. In embodiments, the peptide includes, or is, the amino acid sequence of SEQ ID NO: 556. In embodiments, the peptide includes, or is, the amino acid sequence of SEQ ID NO: 557. In embodiments, the peptide includes, or is, the amino acid sequence of SEQ ID NO: 558. In embodiments, the peptide includes, or is, the amino acid sequence of SEQ ID NO: 559. In embodiments, the peptide includes, or is, the amino acid sequence of SEQ ID NO: 560. In embodiments, the peptide includes, or is, the amino acid sequence of SEQ ID NO: 561. In embodiments, the peptide includes, or is, the amino acid sequence of SEQ ID NO: 562. In embodiments, the peptide includes, or is, the amino acid sequence of SEQ ID NO: 563. In embodiments, the peptide includes, or is, the amino acid sequence of SEQ ID NO: 564. In embodiments, the peptide includes, or is, the amino acid sequence of SEQ ID NO: 565. In embodiments, the peptide includes, or is, the amino acid sequence of SEQ ID NO: 566. In embodiments, the peptide includes, or is, the amino acid sequence of SEQ ID NO: 567. In embodiments, the peptide includes, or is, the amino acid sequence of SEQ ID NO: 568. In embodiments, the peptide includes, or is, the amino acid sequence of SEQ ID NO: 569. In embodiments, the peptide includes, or is, the amino acid sequence of SEQ ID NO: 570. In embodiments, the peptide includes, or is, the amino acid sequence of SEQ ID NO: 571. In embodiments, the peptide includes, or is, the amino acid sequence of SEQ ID NO: 572. In embodiments, the peptide includes, or is, the amino acid sequence of SEQ ID NO: 573. In embodiments, the peptide includes, or is, the amino acid sequence of SEQ ID NO: 574. In embodiments, the peptide includes, or is, the amino acid sequence of SEQ ID NO: 575.

In embodiments, the peptide includes, or is, the amino acid sequence of SEQ ID NO: 576. In embodiments, the peptide includes, or is, the amino acid sequence of SEQ ID NO: 577. In embodiments, the peptide includes, or is, the amino acid sequence of SEQ ID NO: 578. In embodiments, the peptide includes, or is, the amino acid sequence of SEQ ID NO: 579. In embodiments, the peptide includes, or is, the amino acid sequence of SEQ ID NO: 580. In embodiments, the peptide includes, or is, the amino acid sequence of SEQ ID NO: 581. In embodiments, the peptide includes, or is, the amino acid sequence of SEQ ID NO: 582. In embodiments, the peptide includes, or is, the amino acid sequence of SEQ ID NO: 583. In embodiments, the peptide includes, or is, the amino acid sequence of SEQ ID NO: 584. In embodiments, the peptide includes, or is, the amino acid sequence of SEQ ID NO: 585. In embodiments, the peptide includes, or is, the amino acid sequence of SEQ ID NO: 586. In embodiments, the peptide includes, or is, the amino acid sequence of SEQ ID NO: 587. In embodiments, the peptide includes, or is, the amino acid sequence of SEQ ID NO: 588. In embodiments, the peptide includes, or is, the amino acid sequence of SEQ ID NO: 589. In embodiments, the peptide includes, or is, the amino acid sequence of SEQ ID NO: 590. In embodiments, the peptide includes, or is, the amino acid sequence of SEQ ID NO: 591. In embodiments, the peptide includes, or is, the amino acid sequence of SEQ ID NO: 592. In embodiments, the peptide includes, or is, the amino acid sequence of SEQ ID NO: 593. In embodiments, the peptide includes, or is, the amino acid sequence of SEQ ID NO: 594. In embodiments, the peptide includes, or is, the amino acid sequence of SEQ ID NO: 595. In embodiments, the peptide includes, or is, the amino acid sequence of SEQ ID NO: 596. In embodiments, the peptide includes, or is, the amino acid sequence of SEQ ID NO: 597. In embodiments, the peptide includes, or is, the amino acid sequence of SEQ ID NO: 598. In embodiments, the peptide includes, or is, the amino acid sequence of SEQ ID NO: 599. In embodiments, the peptide includes, or is, the amino acid sequence of SEQ ID NO: 600.

In embodiments, the peptide includes, or is, the amino acid sequence of SEQ ID NO: 601. In embodiments, the peptide includes, or is, the amino acid sequence of SEQ ID NO: 602. In embodiments, the peptide includes, or is, the amino acid sequence of SEQ ID NO: 603. In embodiments, the peptide includes, or is, the amino acid sequence of SEQ ID NO: 604. In embodiments, the peptide includes, or is, the amino acid sequence of SEQ ID NO: 605. In embodiments, the peptide includes, or is, the amino acid sequence of SEQ ID NO: 606. In embodiments, the peptide includes, or is, the amino acid sequence of SEQ ID NO: 607. In embodiments, the peptide includes, or is, the amino acid sequence of SEQ ID NO: 608. In embodiments, the peptide includes, or is, the amino acid sequence of SEQ ID NO: 609. In embodiments, the peptide includes, or is, the amino acid sequence of SEQ ID NO: 610. In embodiments, the peptide includes, or is, the amino acid sequence of SEQ ID NO: 611. In embodiments, the peptide includes, or is, the amino acid sequence of SEQ ID NO: 612. In embodiments, the peptide includes, or is, the amino acid sequence of SEQ ID NO: 613. In embodiments, the peptide includes, or is, the amino acid sequence of SEQ ID NO: 614. In embodiments, the peptide includes, or is, the amino acid sequence of SEQ ID NO: 615. In embodiments, the peptide includes, or is, the amino acid sequence of SEQ ID NO: 616. In embodiments, the peptide includes, or is, the amino acid sequence of SEQ ID NO: 617. In embodiments, the peptide includes, or is, the amino acid sequence of SEQ ID NO: 618. In embodiments, the peptide includes, or is, the amino acid sequence of SEQ ID NO: 619. In embodiments, the peptide includes, or is, the amino acid sequence of SEQ ID NO: 620. In embodiments, the peptide includes, or is, the amino acid sequence of SEQ ID NO: 621. In embodiments, the peptide includes, or is, the amino acid sequence of SEQ ID NO: 622. In embodiments, the peptide includes, or is, the amino acid sequence of SEQ ID NO: 623. In embodiments, the peptide includes, or is, the amino acid sequence of SEQ ID NO: 624. In embodiments, the peptide includes, or is, the amino acid sequence of SEQ ID NO: 625.

In embodiments, the peptide includes, or is, the amino acid sequence of SEQ ID NO: 626. In embodiments, the peptide includes, or is, the amino acid sequence of SEQ ID NO: 627. In embodiments, the peptide includes, or is, the amino acid sequence of SEQ ID NO: 628. In embodiments, the peptide includes, or is, the amino acid sequence of SEQ ID NO: 629. In embodiments, the peptide includes, or is, the amino acid sequence of SEQ ID NO: 630. In embodiments, the peptide includes, or is, the amino acid sequence of SEQ ID NO: 631. In embodiments, the peptide includes, or is, the amino acid sequence of SEQ ID NO: 632. In embodiments, the peptide includes, or is, the amino acid sequence of SEQ ID NO: 633. In embodiments, the peptide includes, or is, the amino acid sequence of SEQ ID NO: 634. In embodiments, the peptide includes, or is, the amino acid sequence of SEQ ID NO: 635. In embodiments, the peptide includes, or is, the amino acid sequence of SEQ ID NO: 636. In embodiments, the peptide includes, or is, the amino acid sequence of SEQ ID NO: 637. In embodiments, the peptide includes, or is, the amino acid sequence of SEQ ID NO: 638. In embodiments, the peptide includes, or is, the amino acid sequence of SEQ ID NO: 639. In embodiments, the peptide includes, or is, the amino acid sequence of SEQ ID NO: 640. In embodiments, the peptide includes, or is, the amino acid sequence of SEQ ID NO: 641. In embodiments, the peptide includes, or is, the amino acid sequence of SEQ ID NO: 642. In embodiments, the peptide includes, or is, the amino acid sequence of SEQ ID NO: 643. In embodiments, the peptide includes, or is, the amino acid sequence of SEQ ID NO: 644. In embodiments, the peptide includes, or is, the amino acid sequence of SEQ ID NO: 645. In embodiments, the peptide includes, or is, the amino acid sequence of SEQ ID NO: 646. In embodiments, the peptide includes, or is, the amino acid sequence of SEQ ID NO: 647. In embodiments, the peptide includes, or is, the amino acid sequence of SEQ ID NO: 648. In embodiments, the peptide includes, or is, the amino acid sequence of SEQ ID NO: 649. In embodiments, the peptide includes, or is, the amino acid sequence of SEQ ID NO: 650.

In embodiments, the peptide includes, or is, the amino acid sequence of SEQ ID NO: 651. In embodiments, the peptide includes, or is, the amino acid sequence of SEQ ID NO: 652. In embodiments, the peptide includes, or is, the amino acid sequence of SEQ ID NO: 653. In embodiments, the peptide includes, or is, the amino acid sequence of SEQ ID NO: 654. In embodiments, the peptide includes, or is, the amino acid sequence of SEQ ID NO: 655. In embodiments, the peptide includes, or is, the amino acid sequence of SEQ ID NO: 656. In embodiments, the peptide includes, or is, the amino acid sequence of SEQ ID NO: 657. In embodiments, the peptide includes, or is, the amino acid sequence of SEQ ID NO: 658. In embodiments, the peptide includes, or is, the amino acid sequence of SEQ ID NO: 659. In embodiments, the peptide includes, or is, the amino acid sequence of SEQ ID NO: 660. In embodiments, the peptide includes, or is, the amino acid sequence of SEQ ID NO: 661. In embodiments, the peptide includes, or is, the amino acid sequence of SEQ ID NO: 662. In embodiments, the peptide includes, or is, the amino acid sequence of SEQ ID NO: 663. In embodiments, the peptide includes, or is, the amino acid sequence of SEQ ID NO: 664. In embodiments, the peptide includes, or is, the amino acid sequence of SEQ ID NO: 665. In embodiments, the peptide includes, or is, the amino acid sequence of SEQ ID NO: 666. In embodiments, the peptide includes, or is, the amino acid sequence of SEQ ID NO: 667. In embodiments, the peptide includes, or is, the amino acid sequence of SEQ ID NO: 668. In embodiments, the peptide includes, or is, the amino acid sequence of SEQ ID NO: 669. In embodiments, the peptide includes, or is, the amino acid sequence of SEQ ID NO: 670. In embodiments, the peptide includes, or is, the amino acid sequence of SEQ ID NO: 671. In embodiments, the peptide includes, or is, the amino acid sequence of SEQ ID NO: 672. In embodiments, the peptide includes, or is, the amino acid sequence of SEQ ID NO: 673. In embodiments, the peptide includes, or is, the amino acid sequence of SEQ ID NO: 674. In embodiments, the peptide includes, or is, the amino acid sequence of SEQ ID NO: 675.

In embodiments, the peptide includes, or is, the amino acid sequence of SEQ ID NO: 676. In embodiments, the peptide includes, or is, the amino acid sequence of SEQ ID NO: 677. In embodiments, the peptide includes, or is, the amino acid sequence of SEQ ID NO: 678. In embodiments, the peptide includes, or is, the amino acid sequence of SEQ ID NO: 679. In embodiments, the peptide includes, or is, the amino acid sequence of SEQ ID NO: 680. In embodiments, the peptide includes, or is, the amino acid sequence of SEQ ID NO: 681. In embodiments, the peptide includes, or is, the amino acid sequence of SEQ ID NO: 682. In embodiments, the peptide includes, or is, the amino acid sequence of SEQ ID NO: 683. In embodiments, the peptide includes, or is, the amino acid sequence of SEQ ID NO: 684. In embodiments, the peptide includes, or is, the amino acid sequence of SEQ ID NO: 685. In embodiments, the peptide includes, or is, the amino acid sequence of SEQ ID NO: 686. In embodiments, the peptide includes, or is, the amino acid sequence of SEQ ID NO: 687. In embodiments, the peptide includes, or is, the amino acid sequence of SEQ ID NO: 688. In embodiments, the peptide includes, or is, the amino acid sequence of SEQ ID NO: 689. In embodiments, the peptide includes, or is, the amino acid sequence of SEQ ID NO: 690. In embodiments, the peptide includes, or is, the amino acid sequence of SEQ ID NO: 691. In embodiments, the peptide includes, or is, the amino acid sequence of SEQ ID NO: 692. In embodiments, the peptide includes, or is, the amino acid sequence of SEQ ID NO: 693. In embodiments, the peptide includes, or is, the amino acid sequence of SEQ ID NO: 694. In embodiments, the peptide includes, or is, the amino acid sequence of SEQ ID NO: 695. In embodiments, the peptide includes, or is, the amino acid sequence of SEQ ID NO: 696. In embodiments, the peptide includes, or is, the amino acid sequence of SEQ ID NO: 697. In embodiments, the peptide includes, or is, the amino acid sequence of SEQ ID NO: 698. In embodiments, the peptide includes, or is, the amino acid sequence of SEQ ID NO: 699. In embodiments, the peptide includes, or is, the amino acid sequence of SEQ ID NO: 700.

In embodiments, the peptide includes, or is, the amino acid sequence of SEQ ID NO: 701. In embodiments, the peptide includes, or is, the amino acid sequence of SEQ ID NO: 702. In embodiments, the peptide includes, or is, the amino acid sequence of SEQ ID NO: 703. In embodiments, the peptide includes, or is, the amino acid sequence of SEQ ID NO: 704. In embodiments, the peptide includes, or is, the amino acid sequence of SEQ ID NO: 705. In embodiments, the peptide includes, or is, the amino acid sequence of SEQ ID NO: 706. In embodiments, the peptide includes, or is, the amino acid sequence of SEQ ID NO: 707. In embodiments, the peptide includes, or is, the amino acid sequence of SEQ ID NO: 708. In embodiments, the peptide includes, or is, the amino acid sequence of SEQ ID NO: 709. In embodiments, the peptide includes, or is, the amino acid sequence of SEQ ID NO: 710. In embodiments, the peptide includes, or is, the amino acid sequence of SEQ ID NO: 711. In embodiments, the peptide includes, or is, the amino acid sequence of SEQ ID NO: 712. In embodiments, the peptide includes, or is, the amino acid sequence of SEQ ID NO: 713. In embodiments, the peptide includes, or is, the amino acid sequence of SEQ ID NO: 714. In embodiments, the peptide includes, or is, the amino acid sequence of SEQ ID NO: 715. In embodiments, the peptide includes, or is, the amino acid sequence of SEQ ID NO: 716. In embodiments, the peptide includes, or is, the amino acid sequence of SEQ ID NO: 717. In embodiments, the peptide includes, or is, the amino acid sequence of SEQ ID NO: 718. In embodiments, the peptide includes, or is, the amino acid sequence of SEQ ID NO: 719. In embodiments, the peptide includes, or is, the amino acid sequence of SEQ ID NO: 720. In embodiments, the peptide includes, or is, the amino acid sequence of SEQ ID NO: 721. In embodiments, the peptide includes, or is, the amino acid sequence of SEQ ID NO: 722. In embodiments, the peptide includes, or is, the amino acid sequence of SEQ ID NO: 723. In embodiments, the peptide includes, or is, the amino acid sequence of SEQ ID NO: 724. In embodiments, the peptide includes, or is, the amino acid sequence of SEQ ID NO: 725.

In embodiments, the peptide includes, or is, the amino acid sequence of SEQ ID NO: 726. In embodiments, the peptide includes, or is, the amino acid sequence of SEQ ID NO: 727. In embodiments, the peptide includes, or is, the amino acid sequence of SEQ ID NO: 728. In embodiments, the peptide includes, or is, the amino acid sequence of SEQ ID NO: 729. In embodiments, the peptide includes, or is, the amino acid sequence of SEQ ID NO: 730. In embodiments, the peptide includes, or is, the amino acid sequence of SEQ ID NO: 731. In embodiments, the peptide includes, or is, the amino acid sequence of SEQ ID NO: 732. In embodiments, the peptide includes, or is, the amino acid sequence of SEQ ID NO: 733. In embodiments, the peptide includes, or is, the amino acid sequence of SEQ ID NO: 734. In embodiments, the peptide includes, or is, the amino acid sequence of SEQ ID NO: 735. In embodiments, the peptide includes, or is, the amino acid sequence of SEQ ID NO: 736. In embodiments, the peptide includes, or is, the amino acid sequence of SEQ ID NO: 737. In embodiments, the peptide includes, or is, the amino acid sequence of SEQ ID NO: 738. In embodiments, the peptide includes, or is, the amino acid sequence of SEQ ID NO: 739. In embodiments, the peptide includes, or is, the amino acid sequence of SEQ ID NO: 740. In embodiments, the peptide includes, or is, the amino acid sequence of SEQ ID NO: 741. In embodiments, the peptide includes, or is, the amino acid sequence of SEQ ID NO: 742. In embodiments, the peptide includes, or is, the amino acid sequence of SEQ ID NO: 743. In embodiments, the peptide includes, or is, the amino acid sequence of SEQ ID NO: 744. In embodiments, the peptide includes, or is, the amino acid sequence of SEQ ID NO: 745. In embodiments, the peptide includes, or is, the amino acid sequence of SEQ ID NO: 746. In embodiments, the peptide includes, or is, the amino acid sequence of SEQ ID NO: 747. In embodiments, the peptide includes, or is, the amino acid sequence of SEQ ID NO: 748. In embodiments, the peptide includes, or is, the amino acid sequence of SEQ ID NO: 749. In embodiments, the peptide includes, or is, the amino acid sequence of SEQ ID NO: 750.

In embodiments, the peptide includes, or is, the amino acid sequence of SEQ ID NO: 751. In embodiments, the peptide includes, or is, the amino acid sequence of SEQ ID NO: 752. In embodiments, the peptide includes, or is, the amino acid sequence of SEQ ID NO: 753. In embodiments, the peptide includes, or is, the amino acid sequence of SEQ ID NO: 754. In embodiments, the peptide includes, or is, the amino acid sequence of SEQ ID NO: 755. In embodiments, the peptide includes, or is, the amino acid sequence of SEQ ID NO: 756. In embodiments, the peptide includes, or is, the amino acid sequence of SEQ ID NO: 757. In embodiments, the peptide includes, or is, the amino acid sequence of SEQ ID NO: 758. In embodiments, the peptide includes, or is, the amino acid sequence of SEQ ID NO: 759. In embodiments, the peptide includes, or is, the amino acid sequence of SEQ ID NO: 760. In embodiments, the peptide includes, or is, the amino acid sequence of SEQ ID NO: 761. In embodiments, the peptide includes, or is, the amino acid sequence of SEQ ID NO: 762. In embodiments, the peptide includes, or is, the amino acid sequence of SEQ ID NO: 763. In embodiments, the peptide includes, or is, the amino acid sequence of SEQ ID NO: 764. In embodiments, the peptide includes, or is, the amino acid sequence of SEQ ID NO: 765. In embodiments, the peptide includes, or is, the amino acid sequence of SEQ ID NO: 766. In embodiments, the peptide includes, or is, the amino acid sequence of SEQ ID NO: 767. In embodiments, the peptide includes, or is, the amino acid sequence of SEQ ID NO: 768. In embodiments, the peptide includes, or is, the amino acid sequence of SEQ ID NO: 769. In embodiments, the peptide includes, or is, the amino acid sequence of SEQ ID NO: 770. In embodiments, the peptide includes, or is, the amino acid sequence of SEQ ID NO: 771. In embodiments, the peptide includes, or is, the amino acid sequence of SEQ ID NO: 772. In embodiments, the peptide includes, or is, the amino acid sequence of SEQ ID NO: 773. In embodiments, the peptide includes, or is, the amino acid sequence of SEQ ID NO: 774. In embodiments, the peptide includes, or is, the amino acid sequence of SEQ ID NO: 775.

In embodiments, the peptide includes, or is, the amino acid sequence of SEQ ID NO: 776. In embodiments, the peptide includes, or is, the amino acid sequence of SEQ ID NO: 777. In embodiments, the peptide includes, or is, the amino acid sequence of SEQ ID NO: 778. In embodiments, the peptide includes, or is, the amino acid sequence of SEQ ID NO: 779. In embodiments, the peptide includes, or is, the amino acid sequence of SEQ ID NO: 780. In embodiments, the peptide includes, or is, the amino acid sequence of SEQ ID NO: 781. In embodiments, the peptide includes, or is, the amino acid sequence of SEQ ID NO: 782. In embodiments, the peptide includes, or is, the amino acid sequence of SEQ ID NO: 783. In embodiments, the peptide includes, or is, the amino acid sequence of SEQ ID NO: 784. In embodiments, the peptide includes, or is, the amino acid sequence of SEQ ID NO: 785. In embodiments, the peptide includes, or is, the amino acid sequence of SEQ ID NO: 786. In embodiments, the peptide includes, or is, the amino acid sequence of SEQ ID NO: 787. In embodiments, the peptide includes, or is, the amino acid sequence of SEQ ID NO: 788. In embodiments, the peptide includes, or is, the amino acid sequence of SEQ ID NO: 789. In embodiments, the peptide includes, or is, the amino acid sequence of SEQ ID NO: 790. In embodiments, the peptide includes, or is, the amino acid sequence of SEQ ID NO: 791. In embodiments, the peptide includes, or is, the amino acid sequence of SEQ ID NO: 792. In embodiments, the peptide includes, or is, the amino acid sequence of SEQ ID NO: 793. In embodiments, the peptide includes, or is, the amino acid sequence of SEQ ID NO: 794. In embodiments, the peptide includes, or is, the amino acid sequence of SEQ ID NO: 795. In embodiments, the peptide includes, or is, the amino acid sequence of SEQ ID NO: 796. In embodiments, the peptide includes, or is, the amino acid sequence of SEQ ID NO: 797. In embodiments, the peptide includes, or is, the amino acid sequence of SEQ ID NO: 798. In embodiments, the peptide includes, or is, the amino acid sequence of SEQ ID NO: 799. In embodiments, the peptide includes, or is, the amino acid sequence of SEQ ID NO: 800.

In embodiments, the peptide includes, or is, the amino acid sequence of SEQ ID NO: 801. In embodiments, the peptide includes, or is, the amino acid sequence of SEQ ID NO: 802. In embodiments, the peptide includes, or is, the amino acid sequence of SEQ ID NO: 803. In embodiments, the peptide includes, or is, the amino acid sequence of SEQ ID NO: 804. In embodiments, the peptide includes, or is, the amino acid sequence of SEQ ID NO: 805. In embodiments, the peptide includes, or is, the amino acid sequence of SEQ ID NO: 806. In embodiments, the peptide includes, or is, the amino acid sequence of SEQ ID NO: 807. In embodiments, the peptide includes, or is, the amino acid sequence of SEQ ID NO: 808. In embodiments, the peptide includes, or is, the amino acid sequence of SEQ ID NO: 809. In embodiments, the peptide includes, or is, the amino acid sequence of SEQ ID NO: 810. In embodiments, the peptide includes, or is, the amino acid sequence of SEQ ID NO: 811. In embodiments, the peptide includes, or is, the amino acid sequence of SEQ ID NO: 812. In embodiments, the peptide includes, or is, the amino acid sequence of SEQ ID NO: 813. In embodiments, the peptide includes, or is, the amino acid sequence of SEQ ID NO: 814. In embodiments, the peptide includes, or is, the amino acid sequence of SEQ ID NO: 815. In embodiments, the peptide includes, or is, the amino acid sequence of SEQ ID NO: 816. In embodiments, the peptide includes, or is, the amino acid sequence of SEQ ID NO: 817. In embodiments, the peptide includes, or is, the amino acid sequence of SEQ ID NO: 818. In embodiments, the peptide includes, or is, the amino acid sequence of SEQ ID NO: 819. In embodiments, the peptide includes, or is, the amino acid sequence of SEQ ID NO: 820. In embodiments, the peptide includes, or is, the amino acid sequence of SEQ ID NO: 821. In embodiments, the peptide includes, or is, the amino acid sequence of SEQ ID NO: 822. In embodiments, the peptide includes, or is, the amino acid sequence of SEQ ID NO: 823. In embodiments, the peptide includes, or is, the amino acid sequence of SEQ ID NO: 824. In embodiments, the peptide includes, or is, the amino acid sequence of SEQ ID NO: 825.

In embodiments, the peptide includes, or is, the amino acid sequence of SEQ ID NO: 826. In embodiments, the peptide includes, or is, the amino acid sequence of SEQ ID NO: 827. In embodiments, the peptide includes, or is, the amino acid sequence of SEQ ID NO: 828. In embodiments, the peptide includes, or is, the amino acid sequence of SEQ ID NO: 829. In embodiments, the peptide includes, or is, the amino acid sequence of SEQ ID NO: 830. In embodiments, the peptide includes, or is, the amino acid sequence of SEQ ID NO: 831. In embodiments, the peptide includes, or is, the amino acid sequence of SEQ ID NO: 832. In embodiments, the peptide includes, or is, the amino acid sequence of SEQ ID NO: 833. In embodiments, the peptide includes, or is, the amino acid sequence of SEQ ID NO: 834. In embodiments, the peptide includes, or is, the amino acid sequence of SEQ ID NO: 835. In embodiments, the peptide includes, or is, the amino acid sequence of SEQ ID NO: 836. In embodiments, the peptide includes, or is, the amino acid sequence of SEQ ID NO: 837. In embodiments, the peptide includes, or is, the amino acid sequence of SEQ ID NO: 838. In embodiments, the peptide includes, or is, the amino acid sequence of SEQ ID NO: 839. In embodiments, the peptide includes, or is, the amino acid sequence of SEQ ID NO: 840. In embodiments, the peptide includes, or is, the amino acid sequence of SEQ ID NO: 841. In embodiments, the peptide includes, or is, the amino acid sequence of SEQ ID NO: 842. In embodiments, the peptide includes, or is, the amino acid sequence of SEQ ID NO: 843. In embodiments, the peptide includes, or is, the amino acid sequence of SEQ ID NO: 844. In embodiments, the peptide includes, or is, the amino acid sequence of SEQ ID NO: 845. In embodiments, the peptide includes, or is, the amino acid sequence of SEQ ID NO: 846. In embodiments, the peptide includes, or is, the amino acid sequence of SEQ ID NO: 847. In embodiments, the peptide includes, or is, the amino acid sequence of SEQ ID NO: 848. In embodiments, the peptide includes, or is, the amino acid sequence of SEQ ID NO: 849. In embodiments, the peptide includes, or is, the amino acid sequence of SEQ ID NO: 850.

In embodiments, the peptide includes, or is, the amino acid sequence of SEQ ID NO: 851. In embodiments, the peptide includes, or is, the amino acid sequence of SEQ ID NO: 852. In embodiments, the peptide includes, or is, the amino acid sequence of SEQ ID NO: 853. In embodiments, the peptide includes, or is, the amino acid sequence of SEQ ID NO: 854. In embodiments, the peptide includes, or is, the amino acid sequence of SEQ ID NO: 855. In embodiments, the peptide includes, or is, the amino acid sequence of SEQ ID NO: 856. In embodiments, the peptide includes, or is, the amino acid sequence of SEQ ID NO: 857. In embodiments, the peptide includes, or is, the amino acid sequence of SEQ ID NO: 858. In embodiments, the peptide includes, or is, the amino acid sequence of SEQ ID NO: 859. In embodiments, the peptide includes, or is, the amino acid sequence of SEQ ID NO: 860. In embodiments, the peptide includes, or is, the amino acid sequence of SEQ ID NO: 861. In embodiments, the peptide includes, or is, the amino acid sequence of SEQ ID NO: 862. In embodiments, the peptide includes, or is, the amino acid sequence of SEQ ID NO: 863. In embodiments, the peptide includes, or is, the amino acid sequence of SEQ ID NO: 864. In embodiments, the peptide includes, or is, the amino acid sequence of SEQ ID NO: 865. In embodiments, the peptide includes, or is, the amino acid sequence of SEQ ID NO: 866. In embodiments, the peptide includes, or is, the amino acid sequence of SEQ ID NO: 867. In embodiments, the peptide includes, or is, the amino acid sequence of SEQ ID NO: 868. In embodiments, the peptide includes, or is, the amino acid sequence of SEQ ID NO: 869. In embodiments, the peptide includes, or is, the amino acid sequence of SEQ ID NO: 870. In embodiments, the peptide includes, or is, the amino acid sequence of SEQ ID NO: 871. In embodiments, the peptide includes, or is, the amino acid sequence of SEQ ID NO: 872. In embodiments, the peptide includes, or is, the amino acid sequence of SEQ ID NO: 873. In embodiments, the peptide includes, or is, the amino acid sequence of SEQ ID NO: 874. In embodiments, the peptide includes, or is, the amino acid sequence of SEQ ID NO: 875.

In embodiments, the peptide includes, or is, the amino acid sequence of SEQ ID NO: 876. In embodiments, the peptide includes, or is, the amino acid sequence of SEQ ID NO: 877. In embodiments, the peptide includes, or is, the amino acid sequence of SEQ ID NO: 878. In embodiments, the peptide includes, or is, the amino acid sequence of SEQ ID NO: 879. In embodiments, the peptide includes, or is, the amino acid sequence of SEQ ID NO: 880. In embodiments, the peptide includes, or is, the amino acid sequence of SEQ ID NO: 881. In embodiments, the peptide includes, or is, the amino acid sequence of SEQ ID NO: 882. In embodiments, the peptide includes, or is, the amino acid sequence of SEQ ID NO: 883. In embodiments, the peptide includes, or is, the amino acid sequence of SEQ ID NO: 884. In embodiments, the peptide includes, or is, the amino acid sequence of SEQ ID NO: 885. In embodiments, the peptide includes, or is, the amino acid sequence of SEQ ID NO: 886. In embodiments, the peptide includes, or is, the amino acid sequence of SEQ ID NO: 887. In embodiments, the peptide includes, or is, the amino acid sequence of SEQ ID NO: 888. In embodiments, the peptide includes, or is, the amino acid sequence of SEQ ID NO: 889. In embodiments, the peptide includes, or is, the amino acid sequence of SEQ ID NO: 890. In embodiments, the peptide includes, or is, the amino acid sequence of SEQ ID NO: 891. In embodiments, the peptide includes, or is, the amino acid sequence of SEQ ID NO: 892. In embodiments, the peptide includes, or is, the amino acid sequence of SEQ ID NO: 893. In embodiments, the peptide includes, or is, the amino acid sequence of SEQ ID NO: 894. In embodiments, the peptide includes, or is, the amino acid sequence of SEQ ID NO: 895. In embodiments, the peptide includes, or is, the amino acid sequence of SEQ ID NO: 896. In embodiments, the peptide includes, or is, the amino acid sequence of SEQ ID NO: 897. In embodiments, the peptide includes, or is, the amino acid sequence of SEQ ID NO: 898. In embodiments, the peptide includes, or is, the amino acid sequence of SEQ ID NO: 899. In embodiments, the peptide includes, or is, the amino acid sequence of SEQ ID NO: 900.

In embodiments, the peptide includes, or is, the amino acid sequence of SEQ ID NO: 901. In embodiments, the peptide includes, or is, the amino acid sequence of SEQ ID NO: 902. In embodiments, the peptide includes, or is, the amino acid sequence of SEQ ID NO: 903. In embodiments, the peptide includes, or is, the amino acid sequence of SEQ ID NO: 904. In embodiments, the peptide includes, or is, the amino acid sequence of SEQ ID NO: 905. In embodiments, the peptide includes, or is, the amino acid sequence of SEQ ID NO: 906. In embodiments, the peptide includes, or is, the amino acid sequence of SEQ ID NO: 907. In embodiments, the peptide includes, or is, the amino acid sequence of SEQ ID NO: 908. In embodiments, the peptide includes, or is, the amino acid sequence of SEQ ID NO: 909. In embodiments, the peptide includes, or is, the amino acid sequence of SEQ ID NO: 910. In embodiments, the peptide includes, or is, the amino acid sequence of SEQ ID NO: 911. In embodiments, the peptide includes, or is, the amino acid sequence of SEQ ID NO: 912. In embodiments, the peptide includes, or is, the amino acid sequence of SEQ ID NO: 913. In embodiments, the peptide includes, or is, the amino acid sequence of SEQ ID NO: 914. In embodiments, the peptide includes, or is, the amino acid sequence of SEQ ID NO: 915. In embodiments, the peptide includes, or is, the amino acid sequence of SEQ ID NO: 916. In embodiments, the peptide includes, or is, the amino acid sequence of SEQ ID NO: 917. In embodiments, the peptide includes, or is, the amino acid sequence of SEQ ID NO: 918. In embodiments, the peptide includes, or is, the amino acid sequence of SEQ ID NO: 919. In embodiments, the peptide includes, or is, the amino acid sequence of SEQ ID NO: 920. In embodiments, the peptide includes, or is, the amino acid sequence of SEQ ID NO: 921. In embodiments, the peptide includes, or is, the amino acid sequence of SEQ ID NO: 922. In embodiments, the peptide includes, or is, the amino acid sequence of SEQ ID NO: 923. In embodiments, the peptide includes, or is, the amino acid sequence of SEQ ID NO: 924. In embodiments, the peptide includes, or is, the amino acid sequence of SEQ ID NO: 925.

In embodiments, the peptide includes, or is, the amino acid sequence of SEQ ID NO: 926. In embodiments, the peptide includes, or is, the amino acid sequence of SEQ ID NO: 927. In embodiments, the peptide includes, or is, the amino acid sequence of SEQ ID NO: 928. In embodiments, the peptide includes, or is, the amino acid sequence of SEQ ID NO: 929. In embodiments, the peptide includes, or is, the amino acid sequence of SEQ ID NO: 930. In embodiments, the peptide includes, or is, the amino acid sequence of SEQ ID NO: 931. In embodiments, the peptide includes, or is, the amino acid sequence of SEQ ID NO: 932. In embodiments, the peptide includes, or is, the amino acid sequence of SEQ ID NO: 933. In embodiments, the peptide includes, or is, the amino acid sequence of SEQ ID NO: 934. In embodiments, the peptide includes, or is, the amino acid sequence of SEQ ID NO: 935. In embodiments, the peptide includes, or is, the amino acid sequence of SEQ ID NO: 936. In embodiments, the peptide includes, or is, the amino acid sequence of SEQ ID NO: 937. In embodiments, the peptide includes, or is, the amino acid sequence of SEQ ID NO: 938. In embodiments, the peptide includes, or is, the amino acid sequence of SEQ ID NO: 939. In embodiments, the peptide includes, or is, the amino acid sequence of SEQ ID NO: 940. In embodiments, the peptide includes, or is, the amino acid sequence of SEQ ID NO: 941. In embodiments, the peptide includes, or is, the amino acid sequence of SEQ ID NO: 942. In embodiments, the peptide includes, or is, the amino acid sequence of SEQ ID NO: 943. In embodiments, the peptide includes, or is, the amino acid sequence of SEQ ID NO: 944. In embodiments, the peptide includes, or is, the amino acid sequence of SEQ ID NO: 945. In embodiments, the peptide includes, or is, the amino acid sequence of SEQ ID NO: 946. In embodiments, the peptide includes, or is, the amino acid sequence of SEQ ID NO: 947. In embodiments, the peptide includes, or is, the amino acid sequence of SEQ ID NO: 948. In embodiments, the peptide includes, or is, the amino acid sequence of SEQ ID NO: 949. In embodiments, the peptide includes, or is, the amino acid sequence of SEQ ID NO: 950.

In embodiments, the peptide includes, or is, the amino acid sequence of SEQ ID NO: 951. In embodiments, the peptide includes, or is, the amino acid sequence of SEQ ID NO: 952. In embodiments, the peptide includes, or is, the amino acid sequence of SEQ ID NO: 953. In embodiments, the peptide includes, or is, the amino acid sequence of SEQ ID NO: 954. In embodiments, the peptide includes, or is, the amino acid sequence of SEQ ID NO: 955. In embodiments, the peptide includes, or is, the amino acid sequence of SEQ ID NO: 956. In embodiments, the peptide includes, or is, the amino acid sequence of SEQ ID NO: 957. In embodiments, the peptide includes, or is, the amino acid sequence of SEQ ID NO: 958. In embodiments, the peptide includes, or is, the amino acid sequence of SEQ ID NO: 959. In embodiments, the peptide includes, or is, the amino acid sequence of SEQ ID NO: 960. In embodiments, the peptide includes, or is, the amino acid sequence of SEQ ID NO: 961. In embodiments, the peptide includes, or is, the amino acid sequence of SEQ ID NO: 962. In embodiments, the peptide includes, or is, the amino acid sequence of SEQ ID NO: 963. In embodiments, the peptide includes, or is, the amino acid sequence of SEQ ID NO: 964. In embodiments, the peptide includes, or is, the amino acid sequence of SEQ ID NO: 965. In embodiments, the peptide includes, or is, the amino acid sequence of SEQ ID NO: 966. In embodiments, the peptide includes, or is, the amino acid sequence of SEQ ID NO: 967. In embodiments, the peptide includes, or is, the amino acid sequence of SEQ ID NO: 968. In embodiments, the peptide includes, or is, the amino acid sequence of SEQ ID NO: 969. In embodiments, the peptide includes, or is, the amino acid sequence of SEQ ID NO: 970. In embodiments, the peptide includes, or is, the amino acid sequence of SEQ ID NO: 971. In embodiments, the peptide includes, or is, the amino acid sequence of SEQ ID NO: 972. In embodiments, the peptide includes, or is, the amino acid sequence of SEQ ID NO: 973. In embodiments, the peptide includes, or is, the amino acid sequence of SEQ ID NO: 974. In embodiments, the peptide includes, or is, the amino acid sequence of SEQ ID NO: 975.

In embodiments, the peptide includes, or is, the amino acid sequence of SEQ ID NO: 976. In embodiments, the peptide includes, or is, the amino acid sequence of SEQ ID NO: 977. In embodiments, the peptide includes, or is, the amino acid sequence of SEQ ID NO: 978. In embodiments, the peptide includes, or is, the amino acid sequence of SEQ ID NO: 979. In embodiments, the peptide includes, or is, the amino acid sequence of SEQ ID NO: 980. In embodiments, the peptide includes, or is, the amino acid sequence of SEQ ID NO: 981. In embodiments, the peptide includes, or is, the amino acid sequence of SEQ ID NO: 982. In embodiments, the peptide includes, or is, the amino acid sequence of SEQ ID NO: 983. In embodiments, the peptide includes, or is, the amino acid sequence of SEQ ID NO: 984. In embodiments, the peptide includes, or is, the amino acid sequence of SEQ ID NO: 985. In embodiments, the peptide includes, or is, the amino acid sequence of SEQ ID NO: 986. In embodiments, the peptide includes, or is, the amino acid sequence of SEQ ID NO: 987. In embodiments, the peptide includes, or is, the amino acid sequence of SEQ ID NO: 988. In embodiments, the peptide includes, or is, the amino acid sequence of SEQ ID NO: 989. In embodiments, the peptide includes, or is, the amino acid sequence of SEQ ID NO: 990. In embodiments, the peptide includes, or is, the amino acid sequence of SEQ ID NO: 991. In embodiments, the peptide includes, or is, the amino acid sequence of SEQ ID NO: 992. In embodiments, the peptide includes, or is, the amino acid sequence of SEQ ID NO: 993. In embodiments, the peptide includes, or is, the amino acid sequence of SEQ ID NO: 994. In embodiments, the peptide includes, or is, the amino acid sequence of SEQ ID NO: 995. In embodiments, the peptide includes, or is, the amino acid sequence of SEQ ID NO: 996. In embodiments, the peptide includes, or is, the amino acid sequence of SEQ ID NO: 997. In embodiments, the peptide includes, or is, the amino acid sequence of SEQ ID NO: 998. In embodiments, the peptide includes, or is, the amino acid sequence of SEQ ID NO: 999. In embodiments, the peptide includes, or is, the amino acid sequence of SEQ ID NO: 1000.

In embodiments, the peptide includes, or is, the amino acid sequence of SEQ ID NO: 1001. In embodiments, the peptide includes, or is, the amino acid sequence of SEQ ID NO: 1002. In embodiments, the peptide includes, or is, the amino acid sequence of SEQ ID NO: 1003. In embodiments, the peptide includes, or is, the amino acid sequence of SEQ ID NO: 1004. In embodiments, the peptide includes, or is, the amino acid sequence of SEQ ID NO: 1005. In embodiments, the peptide includes, or is, the amino acid sequence of SEQ ID NO: 1006. In embodiments, the peptide includes, or is, the amino acid sequence of SEQ ID NO: 1007. In embodiments, the peptide includes, or is, the amino acid sequence of SEQ ID NO: 1008. In embodiments, the peptide includes, or is, the amino acid sequence of SEQ ID NO: 1009. In embodiments, the peptide includes, or is, the amino acid sequence of SEQ ID NO: 1010. In embodiments, the peptide includes, or is, the amino acid sequence of SEQ ID NO: 1011. In embodiments, the peptide includes, or is, the amino acid sequence of SEQ ID NO: 1012. In embodiments, the peptide includes, or is, the amino acid sequence of SEQ ID NO: 1013. In embodiments, the peptide includes, or is, the amino acid sequence of SEQ ID NO: 1014. In embodiments, the peptide includes, or is, the amino acid sequence of SEQ ID NO: 1015. In embodiments, the peptide includes, or is, the amino acid sequence of SEQ ID NO: 1016. In embodiments, the peptide includes, or is, the amino acid sequence of SEQ ID NO: 1017. In embodiments, the peptide includes, or is, the amino acid sequence of SEQ ID NO: 1018. In embodiments, the peptide includes, or is, the amino acid sequence of SEQ ID NO: 1019. In embodiments, the peptide includes, or is, the amino acid sequence of SEQ ID NO: 1020. In embodiments, the peptide includes, or is, the amino acid sequence of SEQ ID NO: 1021. In embodiments, the peptide includes, or is, the amino acid sequence of SEQ ID NO: 1022. In embodiments, the peptide includes, or is, the amino acid sequence of SEQ ID NO: 1023. In embodiments, the peptide includes, or is, the amino acid sequence of SEQ ID NO: 1024. In embodiments, the peptide includes, or is, the amino acid sequence of SEQ ID NO: 1025.

In embodiments, the peptide includes, or is, the amino acid sequence of SEQ ID NO: 1026. In embodiments, the peptide includes, or is, the amino acid sequence of SEQ ID NO: 1027. In embodiments, the peptide includes, or is, the amino acid sequence of SEQ ID NO: 1028. In embodiments, the peptide includes, or is, the amino acid sequence of SEQ ID NO: 1029. In embodiments, the peptide includes, or is, the amino acid sequence of SEQ ID NO: 1030. In embodiments, the peptide includes, or is, the amino acid sequence of SEQ ID NO: 1031. In embodiments, the peptide includes, or is, the amino acid sequence of SEQ ID NO: 1032. In embodiments, the peptide includes, or is, the amino acid sequence of SEQ ID NO: 1033. In embodiments, the peptide includes, or is, the amino acid sequence of SEQ ID NO: 1034. In embodiments, the peptide includes, or is, the amino acid sequence of SEQ ID NO: 1035. In embodiments, the peptide includes, or is, the amino acid sequence of SEQ ID NO: 1036. In embodiments, the peptide includes, or is, the amino acid sequence of SEQ ID NO: 1037. In embodiments, the peptide includes, or is, the amino acid sequence of SEQ ID NO: 1038. In embodiments, the peptide includes, or is, the amino acid sequence of SEQ ID NO: 1039. In embodiments, the peptide includes, or is, the amino acid sequence of SEQ ID NO: 1040. In embodiments, the peptide includes, or is, the amino acid sequence of SEQ ID NO: 1041. In embodiments, the peptide includes, or is, the amino acid sequence of SEQ ID NO: 1042. In embodiments, the peptide includes, or is, the amino acid sequence of SEQ ID NO: 1043. In embodiments, the peptide includes, or is, the amino acid sequence of SEQ ID NO: 1044. In embodiments, the peptide includes, or is, the amino acid sequence of SEQ ID NO: 1045. In embodiments, the peptide includes, or is, the amino acid sequence of SEQ ID NO: 1046. In embodiments, the peptide includes, or is, the amino acid sequence of SEQ ID NO: 1047. In embodiments, the peptide includes, or is, the amino acid sequence of SEQ ID NO: 1048. In embodiments, the peptide includes, or is, the amino acid sequence of SEQ ID NO: 1049. In embodiments, the peptide includes, or is, the amino acid sequence of SEQ ID NO: 1050.

In embodiments, the peptide includes, or is, the amino acid sequence of SEQ ID NO: 1051. In embodiments, the peptide includes, or is, the amino acid sequence of SEQ ID NO: 1052. In embodiments, the peptide includes, or is, the amino acid sequence of SEQ ID NO: 1053. In embodiments, the peptide includes, or is, the amino acid sequence of SEQ ID NO: 1054. In embodiments, the peptide includes, or is, the amino acid sequence of SEQ ID NO: 1055. In embodiments, the peptide includes, or is, the amino acid sequence of SEQ ID NO: 1056. In embodiments, the peptide includes, or is, the amino acid sequence of SEQ ID NO: 1057. In embodiments, the peptide includes, or is, the amino acid sequence of SEQ ID NO: 1058. In embodiments, the peptide includes, or is, the amino acid sequence of SEQ ID NO: 1059. In embodiments, the peptide includes, or is, the amino acid sequence of SEQ ID NO: 1060. In embodiments, the peptide includes, or is, the amino acid sequence of SEQ ID NO: 1061. In embodiments, the peptide includes, or is, the amino acid sequence of SEQ ID NO: 1062. In embodiments, the peptide includes, or is, the amino acid sequence of SEQ ID NO: 1063. In embodiments, the peptide includes, or is, the amino acid sequence of SEQ ID NO: 1064. In embodiments, the peptide includes, or is, the amino acid sequence of SEQ ID NO: 1065. In embodiments, the peptide includes, or is, the amino acid sequence of SEQ ID NO: 1066. In embodiments, the peptide includes, or is, the amino acid sequence of SEQ ID NO: 1067. In embodiments, the peptide includes, or is, the amino acid sequence of SEQ ID NO: 1068. In embodiments, the peptide includes, or is, the amino acid sequence of SEQ ID NO: 1069. In embodiments, the peptide includes, or is, the amino acid sequence of SEQ ID NO: 1070. In embodiments, the peptide includes, or is, the amino acid sequence of SEQ ID NO: 1071. In embodiments, the peptide includes, or is, the amino acid sequence of SEQ ID NO: 1072. In embodiments, the peptide includes, or is, the amino acid sequence of SEQ ID NO: 1073. In embodiments, the peptide includes, or is, the amino acid sequence of SEQ ID NO: 1074. In embodiments, the peptide includes, or is, the amino acid sequence of SEQ ID NO: 1075.

In embodiments, the peptide includes, or is, the amino acid sequence of SEQ ID NO: 1076. In embodiments, the peptide includes, or is, the amino acid sequence of SEQ ID NO: 1077. In embodiments, the peptide includes, or is, the amino acid sequence of SEQ ID NO: 1078. In embodiments, the peptide includes, or is, the amino acid sequence of SEQ ID NO: 1079. In embodiments, the peptide includes, or is, the amino acid sequence of SEQ ID NO: 1080. In embodiments, the peptide includes, or is, the amino acid sequence of SEQ ID NO: 1081. In embodiments, the peptide includes, or is, the amino acid sequence of SEQ ID NO: 1082. In embodiments, the peptide includes, or is, the amino acid sequence of SEQ ID NO: 1083. In embodiments, the peptide includes, or is, the amino acid sequence of SEQ ID NO: 1084. In embodiments, the peptide includes, or is, the amino acid sequence of SEQ ID NO: 1085. In embodiments, the peptide includes, or is, the amino acid sequence of SEQ ID NO: 1086. In embodiments, the peptide includes, or is, the amino acid sequence of SEQ ID NO: 1087. In embodiments, the peptide includes, or is, the amino acid sequence of SEQ ID NO: 1088. In embodiments, the peptide includes, or is, the amino acid sequence of SEQ ID NO: 1089. In embodiments, the peptide includes, or is, the amino acid sequence of SEQ ID NO: 1090. In embodiments, the peptide includes, or is, the amino acid sequence of SEQ ID NO: 1091. In embodiments, the peptide includes, or is, the amino acid sequence of SEQ ID NO: 1092. In embodiments, the peptide includes, or is, the amino acid sequence of SEQ ID NO: 1093. In embodiments, the peptide includes, or is, the amino acid sequence of SEQ ID NO: 1094. In embodiments, the peptide includes, or is, the amino acid sequence of SEQ ID NO: 1095. In embodiments, the peptide includes, or is, the amino acid sequence of SEQ ID NO: 1096. In embodiments, the peptide includes, or is, the amino acid sequence of SEQ ID NO: 1097. In embodiments, the peptide includes, or is, the amino acid sequence of SEQ ID NO: 1098. In embodiments, the peptide includes, or is, the amino acid sequence of SEQ ID NO: 1099. In embodiments, the peptide includes, or is, the amino acid sequence of SEQ ID NO: 1100.

In embodiments, the peptide includes, or is, the amino acid sequence of SEQ ID NO: 1101. In embodiments, the peptide includes, or is, the amino acid sequence of SEQ ID NO: 1102. In embodiments, the peptide includes, or is, the amino acid sequence of SEQ ID NO: 1103. In embodiments, the peptide includes, or is, the amino acid sequence of SEQ ID NO: 1104. In embodiments, the peptide includes, or is, the amino acid sequence of SEQ ID NO: 1105. In embodiments, the peptide includes, or is, the amino acid sequence of SEQ ID NO: 1106. In embodiments, the peptide includes, or is, the amino acid sequence of SEQ ID NO: 1107. In embodiments, the peptide includes, or is, the amino acid sequence of SEQ ID NO: 1108. In embodiments, the peptide includes, or is, the amino acid sequence of SEQ ID NO: 1109. In embodiments, the peptide includes, or is, the amino acid sequence of SEQ ID NO: 1110. In embodiments, the peptide includes, or is, the amino acid sequence of SEQ ID NO: 1111. In embodiments, the peptide includes, or is, the amino acid sequence of SEQ ID NO: 1112. In embodiments, the peptide includes, or is, the amino acid sequence of SEQ ID NO: 1113. In embodiments, the peptide includes, or is, the amino acid sequence of SEQ ID NO: 1114. In embodiments, the peptide includes, or is, the amino acid sequence of SEQ ID NO: 1115. In embodiments, the peptide includes, or is, the amino acid sequence of SEQ ID NO: 1116. In embodiments, the peptide includes, or is, the amino acid sequence of SEQ ID NO: 1117. In embodiments, the peptide includes, or is, the amino acid sequence of SEQ ID NO: 1118. In embodiments, the peptide includes, or is, the amino acid sequence of SEQ ID NO: 1119. In embodiments, the peptide includes, or is, the amino acid sequence of SEQ ID NO: 1120. In embodiments, the peptide includes, or is, the amino acid sequence of SEQ ID NO: 1121. In embodiments, the peptide includes, or is, the amino acid sequence of SEQ ID NO: 1122. In embodiments, the peptide includes, or is, the amino acid sequence of SEQ ID NO: 1123. In embodiments, the peptide includes, or is, the amino acid sequence of SEQ ID NO: 1124. In embodiments, the peptide includes, or is, the amino acid sequence of SEQ ID NO: 1125.

In embodiments, the peptide includes, or is, the amino acid sequence of SEQ ID NO: 1126. In embodiments, the peptide includes, or is, the amino acid sequence of SEQ ID NO: 1127. In embodiments, the peptide includes, or is, the amino acid sequence of SEQ ID NO: 1128. In embodiments, the peptide includes, or is, the amino acid sequence of SEQ ID NO: 1129. In embodiments, the peptide includes, or is, the amino acid sequence of SEQ ID NO: 1130. In embodiments, the peptide includes, or is, the amino acid sequence of SEQ ID NO: 1131. In embodiments, the peptide includes, or is, the amino acid sequence of SEQ ID NO: 1132. In embodiments, the peptide includes, or is, the amino acid sequence of SEQ ID NO: 1133. In embodiments, the peptide includes, or is, the amino acid sequence of SEQ ID NO: 1134. In embodiments, the peptide includes, or is, the amino acid sequence of SEQ ID NO: 1135. In embodiments, the peptide includes, or is, the amino acid sequence of SEQ ID NO: 1136. In embodiments, the peptide includes, or is, the amino acid sequence of SEQ ID NO: 1137. In embodiments, the peptide includes, or is, the amino acid sequence of SEQ ID NO: 1138. In embodiments, the peptide includes, or is, the amino acid sequence of SEQ ID NO: 1139. In embodiments, the peptide includes, or is, the amino acid sequence of SEQ ID NO: 1140. In embodiments, the peptide includes, or is, the amino acid sequence of SEQ ID NO: 1141. In embodiments, the peptide includes, or is, the amino acid sequence of SEQ ID NO: 1142. In embodiments, the peptide includes, or is, the amino acid sequence of SEQ ID NO: 1143. In embodiments, the peptide includes, or is, the amino acid sequence of SEQ ID NO: 1144. In embodiments, the peptide includes, or is, the amino acid sequence of SEQ ID NO: 1145. In embodiments, the peptide includes, or is, the amino acid sequence of SEQ ID NO: 1146. In embodiments, the peptide includes, or is, the amino acid sequence of SEQ ID NO: 1147. In embodiments, the peptide includes, or is, the amino acid sequence of SEQ ID NO: 1148. In embodiments, the peptide includes, or is, the amino acid sequence of SEQ ID NO: 1149. In embodiments, the peptide includes, or is, the amino acid sequence of SEQ ID NO: 1150.

In embodiments, the peptide includes, or is, the amino acid sequence of SEQ ID NO: 1151. In embodiments, the peptide includes, or is, the amino acid sequence of SEQ ID NO: 1152. In embodiments, the peptide includes, or is, the amino acid sequence of SEQ ID NO: 1153. In embodiments, the peptide includes, or is, the amino acid sequence of SEQ ID NO: 1154. In embodiments, the peptide includes, or is, the amino acid sequence of SEQ ID NO: 1155. In embodiments, the peptide includes, or is, the amino acid sequence of SEQ ID NO: 1156. In embodiments, the peptide includes, or is, the amino acid sequence of SEQ ID NO: 1157. In embodiments, the peptide includes, or is, the amino acid sequence of SEQ ID NO: 1158. In embodiments, the peptide includes, or is, the amino acid sequence of SEQ ID NO: 1159. In embodiments, the peptide includes, or is, the amino acid sequence of SEQ ID NO: 1160. In embodiments, the peptide includes, or is, the amino acid sequence of SEQ ID NO: 1161. In embodiments, the peptide includes, or is, the amino acid sequence of SEQ ID NO: 1162. In embodiments, the peptide includes, or is, the amino acid sequence of SEQ ID NO: 1163. In embodiments, the peptide includes, or is, the amino acid sequence of SEQ ID NO: 1164. In embodiments, the peptide includes, or is, the amino acid sequence of SEQ ID NO: 1165. In embodiments, the peptide includes, or is, the amino acid sequence of SEQ ID NO: 1166. In embodiments, the peptide includes, or is, the amino acid sequence of SEQ ID NO: 1167. In embodiments, the peptide includes, or is, the amino acid sequence of SEQ ID NO: 1168. In embodiments, the peptide includes, or is, the amino acid sequence of SEQ ID NO: 1169. In embodiments, the peptide includes, or is, the amino acid sequence of SEQ ID NO: 1170. In embodiments, the peptide includes, or is, the amino acid sequence of SEQ ID NO: 1171. In embodiments, the peptide includes, or is, the amino acid sequence of SEQ ID NO: 1172. In embodiments, the peptide includes, or is, the amino acid sequence of SEQ ID NO: 1173. In embodiments, the peptide includes, or is, the amino acid sequence of SEQ ID NO: 1174. In embodiments, the peptide includes, or is, the amino acid sequence of SEQ ID NO: 1175.

In embodiments, the peptide includes, or is, the amino acid sequence of SEQ ID NO: 1176. In embodiments, the peptide includes, or is, the amino acid sequence of SEQ ID NO: 1177. In embodiments, the peptide includes, or is, the amino acid sequence of SEQ ID NO: 1178. In embodiments, the peptide includes, or is, the amino acid sequence of SEQ ID NO: 1179. In embodiments, the peptide includes, or is, the amino acid sequence of SEQ ID NO: 1180. In embodiments, the peptide includes, or is, the amino acid sequence of SEQ ID NO: 1181. In embodiments, the peptide includes, or is, the amino acid sequence of SEQ ID NO: 1182. In embodiments, the peptide includes, or is, the amino acid sequence of SEQ ID NO: 1183. In embodiments, the peptide includes, or is, the amino acid sequence of SEQ ID NO: 1184. In embodiments, the peptide includes, or is, the amino acid sequence of SEQ ID NO: 1185. In embodiments, the peptide includes, or is, the amino acid sequence of SEQ ID NO: 1186. In embodiments, the peptide includes, or is, the amino acid sequence of SEQ ID NO: 1187. In embodiments, the peptide includes, or is, the amino acid sequence of SEQ ID NO: 1188. In embodiments, the peptide includes, or is, the amino acid sequence of SEQ ID NO: 1189. In embodiments, the peptide includes, or is, the amino acid sequence of SEQ ID NO: 1190. In embodiments, the peptide includes, or is, the amino acid sequence of SEQ ID NO: 1191. In embodiments, the peptide includes, or is, the amino acid sequence of SEQ ID NO: 1192. In embodiments, the peptide includes, or is, the amino acid sequence of SEQ ID NO: 1193. In embodiments, the peptide includes, or is, the amino acid sequence of SEQ ID NO: 1194. In embodiments, the peptide includes, or is, the amino acid sequence of SEQ ID NO: 1195. In embodiments, the peptide includes, or is, the amino acid sequence of SEQ ID NO: 1196. In embodiments, the peptide includes, or is, the amino acid sequence of SEQ ID NO: 1197. In embodiments, the peptide includes, or is, the amino acid sequence of SEQ ID NO: 1198. In embodiments, the peptide includes, or is, the amino acid sequence of SEQ ID NO: 1199. In embodiments, the peptide includes, or is, the amino acid sequence of SEQ ID NO: 1200.

In embodiments, the peptide includes, or is, the amino acid sequence of SEQ ID NO: 1201. In embodiments, the peptide includes, or is, the amino acid sequence of SEQ ID NO: 1202. In embodiments, the peptide includes, or is, the amino acid sequence of SEQ ID NO: 1203. In embodiments, the peptide includes, or is, the amino acid sequence of SEQ ID NO: 1204. In embodiments, the peptide includes, or is, the amino acid sequence of SEQ ID NO: 1205. In embodiments, the peptide includes, or is, the amino acid sequence of SEQ ID NO: 1206. In embodiments, the peptide includes, or is, the amino acid sequence of SEQ ID NO: 1207. In embodiments, the peptide includes, or is, the amino acid sequence of SEQ ID NO: 1208. In embodiments, the peptide includes, or is, the amino acid sequence of SEQ ID NO: 1209. In embodiments, the peptide includes, or is, the amino acid sequence of SEQ ID NO: 1210. In embodiments, the peptide includes, or is, the amino acid sequence of SEQ ID NO: 1211. In embodiments, the peptide includes, or is, the amino acid sequence of SEQ ID NO: 1212. In embodiments, the peptide includes, or is, the amino acid sequence of SEQ ID NO: 1213. In embodiments, the peptide includes, or is, the amino acid sequence of SEQ ID NO: 1214. In embodiments, the peptide includes, or is, the amino acid sequence of SEQ ID NO: 1215. In embodiments, the peptide includes, or is, the amino acid sequence of SEQ ID NO: 1216. In embodiments, the peptide includes, or is, the amino acid sequence of SEQ ID NO: 1217. In embodiments, the peptide includes, or is, the amino acid sequence of SEQ ID NO: 1218. In embodiments, the peptide includes, or is, the amino acid sequence of SEQ ID NO: 1219. In embodiments, the peptide includes, or is, the amino acid sequence of SEQ ID NO: 1220. In embodiments, the peptide includes, or is, the amino acid sequence of SEQ ID NO: 1221. In embodiments, the peptide includes, or is, the amino acid sequence of SEQ ID NO: 1222. In embodiments, the peptide includes, or is, the amino acid sequence of SEQ ID NO: 1223. In embodiments, the peptide includes, or is, the amino acid sequence of SEQ ID NO: 1224. In embodiments, the peptide includes, or is, the amino acid sequence of SEQ ID NO: 1225.

In embodiments, the peptide includes, or is, the amino acid sequence of SEQ ID NO: 1226. In embodiments, the peptide includes, or is, the amino acid sequence of SEQ ID NO: 1227. In embodiments, the peptide includes, or is, the amino acid sequence of SEQ ID NO: 1228. In embodiments, the peptide includes, or is, the amino acid sequence of SEQ ID NO: 1229. In embodiments, the peptide includes, or is, the amino acid sequence of SEQ ID NO: 1230. In embodiments, the peptide includes, or is, the amino acid sequence of SEQ ID NO: 1231. In embodiments, the peptide includes, or is, the amino acid sequence of SEQ ID NO: 1232. In embodiments, the peptide includes, or is, the amino acid sequence of SEQ ID NO: 1233. In embodiments, the peptide includes, or is, the amino acid sequence of SEQ ID NO: 1234. In embodiments, the peptide includes, or is, the amino acid sequence of SEQ ID NO: 1235. In embodiments, the peptide includes, or is, the amino acid sequence of SEQ ID NO: 1236. In embodiments, the peptide includes, or is, the amino acid sequence of SEQ ID NO: 1237. In embodiments, the peptide includes, or is, the amino acid sequence of SEQ ID NO: 1238. In embodiments, the peptide includes, or is, the amino acid sequence of SEQ ID NO: 1239. In embodiments, the peptide includes, or is, the amino acid sequence of SEQ ID NO: 1240. In embodiments, the peptide includes, or is, the amino acid sequence of SEQ ID NO: 1241. In embodiments, the peptide includes, or is, the amino acid sequence of SEQ ID NO: 1242. In embodiments, the peptide includes, or is, the amino acid sequence of SEQ ID NO: 1243. In embodiments, the peptide includes, or is, the amino acid sequence of SEQ ID NO: 1244. In embodiments, the peptide includes, or is, the amino acid sequence of SEQ ID NO: 1245. In embodiments, the peptide includes, or is, the amino acid sequence of SEQ ID NO: 1246. In embodiments, the peptide includes, or is, the amino acid sequence of SEQ ID NO: 1247. In embodiments, the peptide includes, or is, the amino acid sequence of SEQ ID NO: 1248. In embodiments, the peptide includes, or is, the amino acid sequence of SEQ ID NO: 1249. In embodiments, the peptide includes, or is, the amino acid sequence of SEQ ID NO: 1250.

In embodiments, the peptide includes, or is, the amino acid sequence of SEQ ID NO: 1251. In embodiments, the peptide includes, or is, the amino acid sequence of SEQ ID NO: 1252. In embodiments, the peptide includes, or is, the amino acid sequence of SEQ ID NO: 1253. In embodiments, the peptide includes, or is, the amino acid sequence of SEQ ID NO: 1254. In embodiments, the peptide includes, or is, the amino acid sequence of SEQ ID NO: 1255. In embodiments, the peptide includes, or is, the amino acid sequence of SEQ ID NO: 1256. In embodiments, the peptide includes, or is, the amino acid sequence of SEQ ID NO: 1257. In embodiments, the peptide includes, or is, the amino acid sequence of SEQ ID NO: 1258. In embodiments, the peptide includes, or is, the amino acid sequence of SEQ ID NO: 1259. In embodiments, the peptide includes, or is, the amino acid sequence of SEQ ID NO: 1260. In embodiments, the peptide includes, or is, the amino acid sequence of SEQ ID NO: 1261. In embodiments, the peptide includes, or is, the amino acid sequence of SEQ ID NO: 1262. In embodiments, the peptide includes, or is, the amino acid sequence of SEQ ID NO: 1263. In embodiments, the peptide includes, or is, the amino acid sequence of SEQ ID NO: 1264. In embodiments, the peptide includes, or is, the amino acid sequence of SEQ ID NO: 1265. In embodiments, the peptide includes, or is, the amino acid sequence of SEQ ID NO: 1266. In embodiments, the peptide includes, or is, the amino acid sequence of SEQ ID NO: 1267. In embodiments, the peptide includes, or is, the amino acid sequence of SEQ ID NO: 1268. In embodiments, the peptide includes, or is, the amino acid sequence of SEQ ID NO: 1269. In embodiments, the peptide includes, or is, the amino acid sequence of SEQ ID NO: 1270. In embodiments, the peptide includes, or is, the amino acid sequence of SEQ ID NO: 1271. In embodiments, the peptide includes, or is, the amino acid sequence of SEQ ID NO: 1272. In embodiments, the peptide includes, or is, the amino acid sequence of SEQ ID NO: 1273. In embodiments, the peptide includes, or is, the amino acid sequence of SEQ ID NO: 1274. In embodiments, the peptide includes, or is, the amino acid sequence of SEQ ID NO: 1275.

In embodiments, the peptide includes, or is, the amino acid sequence of SEQ ID NO: 1276. In embodiments, the peptide includes, or is, the amino acid sequence of SEQ ID NO: 1277. In embodiments, the peptide includes, or is, the amino acid sequence of SEQ ID NO: 1278. In embodiments, the peptide includes, or is, the amino acid sequence of SEQ ID NO: 1279. In embodiments, the peptide includes, or is, the amino acid sequence of SEQ ID NO: 1280. In embodiments, the peptide includes, or is, the amino acid sequence of SEQ ID NO: 1281. In embodiments, the peptide includes, or is, the amino acid sequence of SEQ ID NO: 1282. In embodiments, the peptide includes, or is, the amino acid sequence of SEQ ID NO: 1283. In embodiments, the peptide includes, or is, the amino acid sequence of SEQ ID NO: 1284. In embodiments, the peptide includes, or is, the amino acid sequence of SEQ ID NO: 1285. In embodiments, the peptide includes, or is, the amino acid sequence of SEQ ID NO: 1286. In embodiments, the peptide includes, or is, the amino acid sequence of SEQ ID NO: 1287. In embodiments, the peptide includes, or is, the amino acid sequence of SEQ ID NO: 1288. In embodiments, the peptide includes, or is, the amino acid sequence of SEQ ID NO: 1289. In embodiments, the peptide includes, or is, the amino acid sequence of SEQ ID NO: 1290. In embodiments, the peptide includes, or is, the amino acid sequence of SEQ ID NO: 1291. In embodiments, the peptide includes, or is, the amino acid sequence of SEQ ID NO: 1292. In embodiments, the peptide includes, or is, the amino acid sequence of SEQ ID NO: 1293. In embodiments, the peptide includes, or is, the amino acid sequence of SEQ ID NO: 1294. In embodiments, the peptide includes, or is, the amino acid sequence of SEQ ID NO: 1295. In embodiments, the peptide includes, or is, the amino acid sequence of SEQ ID NO: 1296. In embodiments, the peptide includes, or is, the amino acid sequence of SEQ ID NO: 1297. In embodiments, the peptide includes, or is, the amino acid sequence of SEQ ID NO: 1298. In embodiments, the peptide includes, or is, the amino acid sequence of SEQ ID NO: 1299. In embodiments, the peptide includes, or is, the amino acid sequence of SEQ ID NO: 1300.

In embodiments, the peptide includes, or is, the amino acid sequence of SEQ ID NO: 1301. In embodiments, the peptide includes, or is, the amino acid sequence of SEQ ID NO: 1302. In embodiments, the peptide includes, or is, the amino acid sequence of SEQ ID NO: 1303. In embodiments, the peptide includes, or is, the amino acid sequence of SEQ ID NO: 1304. In embodiments, the peptide includes, or is, the amino acid sequence of SEQ ID NO: 1305. In embodiments, the peptide includes, or is, the amino acid sequence of SEQ ID NO: 1306. In embodiments, the peptide includes, or is, the amino acid sequence of SEQ ID NO: 1307. In embodiments, the peptide includes, or is, the amino acid sequence of SEQ ID NO: 1308. In embodiments, the peptide includes, or is, the amino acid sequence of SEQ ID NO: 1309. In embodiments, the peptide includes, or is, the amino acid sequence of SEQ ID NO: 1310. In embodiments, the peptide includes, or is, the amino acid sequence of SEQ ID NO: 1311. In embodiments, the peptide includes, or is, the amino acid sequence of SEQ ID NO: 1312. In embodiments, the peptide includes, or is, the amino acid sequence of SEQ ID NO: 1313. In embodiments, the peptide includes, or is, the amino acid sequence of SEQ ID NO: 1314. In embodiments, the peptide includes, or is, the amino acid sequence of SEQ ID NO: 1315. In embodiments, the peptide includes, or is, the amino acid sequence of SEQ ID NO: 1316. In embodiments, the peptide includes, or is, the amino acid sequence of SEQ ID NO: 1317. In embodiments, the peptide includes, or is, the amino acid sequence of SEQ ID NO: 1318. In embodiments, the peptide includes, or is, the amino acid sequence of SEQ ID NO: 1319. In embodiments, the peptide includes, or is, the amino acid sequence of SEQ ID NO: 1320. In embodiments, the peptide includes, or is, the amino acid sequence of SEQ ID NO: 1321. In embodiments, the peptide includes, or is, the amino acid sequence of SEQ ID NO: 1322. In embodiments, the peptide includes, or is, the amino acid sequence of SEQ ID NO: 1323. In embodiments, the peptide includes, or is, the amino acid sequence of SEQ ID NO: 1324. In embodiments, the peptide includes, or is, the amino acid sequence of SEQ ID NO: 1325.

In embodiments, the peptide includes, or is, the amino acid sequence of SEQ ID NO: 1326. In embodiments, the peptide includes, or is, the amino acid sequence of SEQ ID NO: 1327. In embodiments, the peptide includes, or is, the amino acid sequence of SEQ ID NO: 1328. In embodiments, the peptide includes, or is, the amino acid sequence of SEQ ID NO: 1329. In embodiments, the peptide includes, or is, the amino acid sequence of SEQ ID NO: 1330. In embodiments, the peptide includes, or is, the amino acid sequence of SEQ ID NO: 1331. In embodiments, the peptide includes, or is, the amino acid sequence of SEQ ID NO: 1332. In embodiments, the peptide includes, or is, the amino acid sequence of SEQ ID NO: 1333. In embodiments, the peptide includes, or is, the amino acid sequence of SEQ ID NO: 1334. In embodiments, the peptide includes, or is, the amino acid sequence of SEQ ID NO: 1335. In embodiments, the peptide includes, or is, the amino acid sequence of SEQ ID NO: 1336. In embodiments, the peptide includes, or is, the amino acid sequence of SEQ ID NO: 1337. In embodiments, the peptide includes, or is, the amino acid sequence of SEQ ID NO: 1338. In embodiments, the peptide includes, or is, the amino acid sequence of SEQ ID NO: 1339. In embodiments, the peptide includes, or is, the amino acid sequence of SEQ ID NO: 1340. In embodiments, the peptide includes, or is, the amino acid sequence of SEQ ID NO: 1341. In embodiments, the peptide includes, or is, the amino acid sequence of SEQ ID NO: 1342. In embodiments, the peptide includes, or is, the amino acid sequence of SEQ ID NO: 1343. In embodiments, the peptide includes, or is, the amino acid sequence of SEQ ID NO: 1344. In embodiments, the peptide includes, or is, the amino acid sequence of SEQ ID NO: 1345. In embodiments, the peptide includes, or is, the amino acid sequence of SEQ ID NO: 1346. In embodiments, the peptide includes, or is, the amino acid sequence of SEQ ID NO: 1347. In embodiments, the peptide includes, or is, the amino acid sequence of SEQ ID NO: 1348. In embodiments, the peptide includes, or is, the amino acid sequence of SEQ ID NO: 1349. In embodiments, the peptide includes, or is, the amino acid sequence of SEQ ID NO: 1350.

In embodiments, the peptide includes, or is, the amino acid sequence of SEQ ID NO: 1351. In embodiments, the peptide includes, or is, the amino acid sequence of SEQ ID NO: 1352. In embodiments, the peptide includes, or is, the amino acid sequence of SEQ ID NO: 1353. In embodiments, the peptide includes, or is, the amino acid sequence of SEQ ID NO: 1354. In embodiments, the peptide includes, or is, the amino acid sequence of SEQ ID NO: 1355. In embodiments, the peptide includes, or is, the amino acid sequence of SEQ ID NO: 1356. In embodiments, the peptide includes, or is, the amino acid sequence of SEQ ID NO: 1357. In embodiments, the peptide includes, or is, the amino acid sequence of SEQ ID NO: 1358. In embodiments, the peptide includes, or is, the amino acid sequence of SEQ ID NO: 1359. In embodiments, the peptide includes, or is, the amino acid sequence of SEQ ID NO: 1360. In embodiments, the peptide includes, or is, the amino acid sequence of SEQ ID NO: 1361. In embodiments, the peptide includes, or is, the amino acid sequence of SEQ ID NO: 1362. In embodiments, the peptide includes, or is, the amino acid sequence of SEQ ID NO: 1363. In embodiments, the peptide includes, or is, the amino acid sequence of SEQ ID NO: 1364. In embodiments, the peptide includes, or is, the amino acid sequence of SEQ ID NO: 1365. In embodiments, the peptide includes, or is, the amino acid sequence of SEQ ID NO: 1366. In embodiments, the peptide includes, or is, the amino acid sequence of SEQ ID NO: 1367. In embodiments, the peptide includes, or is, the amino acid sequence of SEQ ID NO: 1368. In embodiments, the peptide includes, or is, the amino acid sequence of SEQ ID NO: 1369. In embodiments, the peptide includes, or is, the amino acid sequence of SEQ ID NO: 1370. In embodiments, the peptide includes, or is, the amino acid sequence of SEQ ID NO: 1371. In embodiments, the peptide includes, or is, the amino acid sequence of SEQ ID NO: 1372. In embodiments, the peptide includes, or is, the amino acid sequence of SEQ ID NO: 1373. In embodiments, the peptide includes, or is, the amino acid sequence of SEQ ID NO: 1374. In embodiments, the peptide includes, or is, the amino acid sequence of SEQ ID NO: 1375.

In embodiments, the peptide includes, or is, the amino acid sequence of SEQ ID NO: 1376. In embodiments, the peptide includes, or is, the amino acid sequence of SEQ ID NO: 1377. In embodiments, the peptide includes, or is, the amino acid sequence of SEQ ID NO: 1378. In embodiments, the peptide includes, or is, the amino acid sequence of SEQ ID NO: 1379. In embodiments, the peptide includes, or is, the amino acid sequence of SEQ ID NO: 1380. In embodiments, the peptide includes, or is, the amino acid sequence of SEQ ID NO: 1381. In embodiments, the peptide includes, or is, the amino acid sequence of SEQ ID NO: 1382. In embodiments, the peptide includes, or is, the amino acid sequence of SEQ ID NO: 1383. In embodiments, the peptide includes, or is, the amino acid sequence of SEQ ID NO: 1384. In embodiments, the peptide includes, or is, the amino acid sequence of SEQ ID NO: 1385. In embodiments, the peptide includes, or is, the amino acid sequence of SEQ ID NO: 1386. In embodiments, the peptide includes, or is, the amino acid sequence of SEQ ID NO: 1387. In embodiments, the peptide includes, or is, the amino acid sequence of SEQ ID NO: 1388. In embodiments, the peptide includes, or is, the amino acid sequence of SEQ ID NO: 1389. In embodiments, the peptide includes, or is, the amino acid sequence of SEQ ID NO: 1390. In embodiments, the peptide includes, or is, the amino acid sequence of SEQ ID NO: 1391. In embodiments, the peptide includes, or is, the amino acid sequence of SEQ ID NO: 1392. In embodiments, the peptide includes, or is, the amino acid sequence of SEQ ID NO: 1393. In embodiments, the peptide includes, or is, the amino acid sequence of SEQ ID NO: 1394. In embodiments, the peptide includes, or is, the amino acid sequence of SEQ ID NO: 1395. In embodiments, the peptide includes, or is, the amino acid sequence of SEQ ID NO: 1396. In embodiments, the peptide includes, or is, the amino acid sequence of SEQ ID NO: 1397. In embodiments, the peptide includes, or is, the amino acid sequence of SEQ ID NO: 1398. In embodiments, the peptide includes, or is, the amino acid sequence of SEQ ID NO: 1399. In embodiments, the peptide includes, or is, the amino acid sequence of SEQ ID NO: 1400.

In embodiments, the peptide includes, or is, the amino acid sequence of SEQ ID NO: 1401. In embodiments, the peptide includes, or is, the amino acid sequence of SEQ ID NO: 1402. In embodiments, the peptide includes, or is, the amino acid sequence of SEQ ID NO: 1403. In embodiments, the peptide includes, or is, the amino acid sequence of SEQ ID NO: 1404. In embodiments, the peptide includes, or is, the amino acid sequence of SEQ ID NO: 1405. In embodiments, the peptide includes, or is, the amino acid sequence of SEQ ID NO: 1406. In embodiments, the peptide includes, or is, the amino acid sequence of SEQ ID NO: 1407. In embodiments, the peptide includes, or is, the amino acid sequence of SEQ ID NO: 1408. In embodiments, the peptide includes, or is, the amino acid sequence of SEQ ID NO: 1409. In embodiments, the peptide includes, or is, the amino acid sequence of SEQ ID NO: 1410. In embodiments, the peptide includes, or is, the amino acid sequence of SEQ ID NO: 1411. In embodiments, the peptide includes, or is, the amino acid sequence of SEQ ID NO: 1412. In embodiments, the peptide includes, or is, the amino acid sequence of SEQ ID NO: 1413. In embodiments, the peptide includes, or is, the amino acid sequence of SEQ ID NO: 1414. In embodiments, the peptide includes, or is, the amino acid sequence of SEQ ID NO: 1415. In embodiments, the peptide includes, or is, the amino acid sequence of SEQ ID NO: 1416. In embodiments, the peptide includes, or is, the amino acid sequence of SEQ ID NO: 1417. In embodiments, the peptide includes, or is, the amino acid sequence of SEQ ID NO: 1418. In embodiments, the peptide includes, or is, the amino acid sequence of SEQ ID NO: 1419. In embodiments, the peptide includes, or is, the amino acid sequence of SEQ ID NO: 1420. In embodiments, the peptide includes, or is, the amino acid sequence of SEQ ID NO: 1421. In embodiments, the peptide includes, or is, the amino acid sequence of SEQ ID NO: 1422. In embodiments, the peptide includes, or is, the amino acid sequence of SEQ ID NO: 1423. In embodiments, the peptide includes, or is, the amino acid sequence of SEQ ID NO: 1424. In embodiments, the peptide includes, or is, the amino acid sequence of SEQ ID NO: 1425.

In embodiments, the peptide includes, or is, the amino acid sequence of SEQ ID NO: 1426. In embodiments, the peptide includes, or is, the amino acid sequence of SEQ ID NO: 1427. In embodiments, the peptide includes, or is, the amino acid sequence of SEQ ID NO: 1428. In embodiments, the peptide includes, or is, the amino acid sequence of SEQ ID NO: 1429. In embodiments, the peptide includes, or is, the amino acid sequence of SEQ ID NO: 1430. In embodiments, the peptide includes, or is, the amino acid sequence of SEQ ID NO: 1431. In embodiments, the peptide includes, or is, the amino acid sequence of SEQ ID NO: 1432. In embodiments, the peptide includes, or is, the amino acid sequence of SEQ ID NO: 1433. In embodiments, the peptide includes, or is, the amino acid sequence of SEQ ID NO: 1434. In embodiments, the peptide includes, or is, the amino acid sequence of SEQ ID NO: 1435. In embodiments, the peptide includes, or is, the amino acid sequence of SEQ ID NO: 1436. In embodiments, the peptide includes, or is, the amino acid sequence of SEQ ID NO: 1437. In embodiments, the peptide includes, or is, the amino acid sequence of SEQ ID NO: 1438. In embodiments, the peptide includes, or is, the amino acid sequence of SEQ ID NO: 1439. In embodiments, the peptide includes, or is, the amino acid sequence of SEQ ID NO: 1440. In embodiments, the peptide includes, or is, the amino acid sequence of SEQ ID NO: 1441. In embodiments, the peptide includes, or is, the amino acid sequence of SEQ ID NO: 1442. In embodiments, the peptide includes, or is, the amino acid sequence of SEQ ID NO: 1443. In embodiments, the peptide includes, or is, the amino acid sequence of SEQ ID NO: 1444. In embodiments, the peptide includes, or is, the amino acid sequence of SEQ ID NO: 1445. In embodiments, the peptide includes, or is, the amino acid sequence of SEQ ID NO: 1446. In embodiments, the peptide includes, or is, the amino acid sequence of SEQ ID NO: 1447. In embodiments, the peptide includes, or is, the amino acid sequence of SEQ ID NO: 1448. In embodiments, the peptide includes, or is, the amino acid sequence of SEQ ID NO: 1449. In embodiments, the peptide includes, or is, the amino acid sequence of SEQ ID NO: 1450.

In embodiments, the peptide includes, or is, the amino acid sequence of SEQ ID NO: 1451. In embodiments, the peptide includes, or is, the amino acid sequence of SEQ ID NO: 1452. In embodiments, the peptide includes, or is, the amino acid sequence of SEQ ID NO: 1453. In embodiments, the peptide includes, or is, the amino acid sequence of SEQ ID NO: 1454. In embodiments, the peptide includes, or is, the amino acid sequence of SEQ ID NO: 1455. In embodiments, the peptide includes, or is, the amino acid sequence of SEQ ID NO: 1456. In embodiments, the peptide includes, or is, the amino acid sequence of SEQ ID NO: 1457. In embodiments, the peptide includes, or is, the amino acid sequence of SEQ ID NO: 1458. In embodiments, the peptide includes, or is, the amino acid sequence of SEQ ID NO: 1459. In embodiments, the peptide includes, or is, the amino acid sequence of SEQ ID NO: 1460. In embodiments, the peptide includes, or is, the amino acid sequence of SEQ ID NO: 1461. In embodiments, the peptide includes, or is, the amino acid sequence of SEQ ID NO: 1462. In embodiments, the peptide includes, or is, the amino acid sequence of SEQ ID NO: 1463. In embodiments, the peptide includes, or is, the amino acid sequence of SEQ ID NO: 1464. In embodiments, the peptide includes, or is, the amino acid sequence of SEQ ID NO: 1465. In embodiments, the peptide includes, or is, the amino acid sequence of SEQ ID NO: 1466. In embodiments, the peptide includes, or is, the amino acid sequence of SEQ ID NO: 1467. In embodiments, the peptide includes, or is, the amino acid sequence of SEQ ID NO: 1468. In embodiments, the peptide includes, or is, the amino acid sequence of SEQ ID NO: 1469. In embodiments, the peptide includes, or is, the amino acid sequence of SEQ ID NO: 1470. In embodiments, the peptide includes, or is, the amino acid sequence of SEQ ID NO: 1471. In embodiments, the peptide includes, or is, the amino acid sequence of SEQ ID NO: 1472. In embodiments, the peptide includes, or is, the amino acid sequence of SEQ ID NO: 1473. In embodiments, the peptide includes, or is, the amino acid sequence of SEQ ID NO: 1474. In embodiments, the peptide includes, or is, the amino acid sequence of SEQ ID NO: 1475.

In embodiments, the peptide includes, or is, the amino acid sequence of SEQ ID NO: 1476. In embodiments, the peptide includes, or is, the amino acid sequence of SEQ ID NO: 1477. In embodiments, the peptide includes, or is, the amino acid sequence of SEQ ID NO: 1478. In embodiments, the peptide includes, or is, the amino acid sequence of SEQ ID NO: 1479. In embodiments, the peptide includes, or is, the amino acid sequence of SEQ ID NO: 1480. In embodiments, the peptide includes, or is, the amino acid sequence of SEQ ID NO: 1481. In embodiments, the peptide includes, or is, the amino acid sequence of SEQ ID NO: 1482. In embodiments, the peptide includes, or is, the amino acid sequence of SEQ ID NO: 1483. In embodiments, the peptide includes, or is, the amino acid sequence of SEQ ID NO: 1484. In embodiments, the peptide includes, or is, the amino acid sequence of SEQ ID NO: 1485. In embodiments, the peptide includes, or is, the amino acid sequence of SEQ ID NO: 1486. In embodiments, the peptide includes, or is, the amino acid sequence of SEQ ID NO: 1487. In embodiments, the peptide includes, or is, the amino acid sequence of SEQ ID NO: 1488. In embodiments, the peptide includes, or is, the amino acid sequence of SEQ ID NO: 1489. In embodiments, the peptide includes, or is, the amino acid sequence of SEQ ID NO: 1490. In embodiments, the peptide includes, or is, the amino acid sequence of SEQ ID NO: 1491. In embodiments, the peptide includes, or is, the amino acid sequence of SEQ ID NO: 1492. In embodiments, the peptide includes, or is, the amino acid sequence of SEQ ID NO: 1493. In embodiments, the peptide includes, or is, the amino acid sequence of SEQ ID NO: 1494. In embodiments, the peptide includes, or is, the amino acid sequence of SEQ ID NO: 1495. In embodiments, the peptide includes, or is, the amino acid sequence of SEQ ID NO: 1496. In embodiments, the peptide includes, or is, the amino acid sequence of SEQ ID NO: 1497. In embodiments, the peptide includes, or is, the amino acid sequence of SEQ ID NO: 1498. In embodiments, the peptide includes, or is, the amino acid sequence of SEQ ID NO: 1499. In embodiments, the peptide includes, or is, the amino acid sequence of SEQ ID NO: 1500.

In embodiments, the peptide includes, or is, the amino acid sequence of SEQ ID NO: 1501. In embodiments, the peptide includes, or is, the amino acid sequence of SEQ ID NO: 1502. In embodiments, the peptide includes, or is, the amino acid sequence of SEQ ID NO: 1503. In embodiments, the peptide includes, or is, the amino acid sequence of SEQ ID NO: 1504. In embodiments, the peptide includes, or is, the amino acid sequence of SEQ ID NO: 1505. In embodiments, the peptide includes, or is, the amino acid sequence of SEQ ID NO: 1506. In embodiments, the peptide includes, or is, the amino acid sequence of SEQ ID NO: 1507. In embodiments, the peptide includes, or is, the amino acid sequence of SEQ ID NO: 1508. In embodiments, the peptide includes, or is, the amino acid sequence of SEQ ID NO: 1509. In embodiments, the peptide includes, or is, the amino acid sequence of SEQ ID NO: 1510. In embodiments, the peptide includes, or is, the amino acid sequence of SEQ ID NO: 1511. In embodiments, the peptide includes, or is, the amino acid sequence of SEQ ID NO: 1512. In embodiments, the peptide includes, or is, the amino acid sequence of SEQ ID NO: 1513. In embodiments, the peptide includes, or is, the amino acid sequence of SEQ ID NO: 1514. In embodiments, the peptide includes, or is, the amino acid sequence of SEQ ID NO: 1515. In embodiments, the peptide includes, or is, the amino acid sequence of SEQ ID NO: 1516. In embodiments, the peptide includes, or is, the amino acid sequence of SEQ ID NO: 1517. In embodiments, the peptide includes, or is, the amino acid sequence of SEQ ID NO: 1518. In embodiments, the peptide includes, or is, the amino acid sequence of SEQ ID NO: 1519. In embodiments, the peptide includes, or is, the amino acid sequence of SEQ ID NO: 1520. In embodiments, the peptide includes, or is, the amino acid sequence of SEQ ID NO: 1521. In embodiments, the peptide includes, or is, the amino acid sequence of SEQ ID NO: 1522. In embodiments, the peptide includes, or is, the amino acid sequence of SEQ ID NO: 1523. In embodiments, the peptide includes, or is, the amino acid sequence of SEQ ID NO: 1524. In embodiments, the peptide includes, or is, the amino acid sequence of SEQ ID NO: 1525.

In embodiments, the peptide includes, or is, the amino acid sequence of SEQ ID NO: 1526. In embodiments, the peptide includes, or is, the amino acid sequence of SEQ ID NO: 1527. In embodiments, the peptide includes, or is, the amino acid sequence of SEQ ID NO: 1528. In embodiments, the peptide includes, or is, the amino acid sequence of SEQ ID NO: 1529. In embodiments, the peptide includes, or is, the amino acid sequence of SEQ ID NO: 1530. In embodiments, the peptide includes, or is, the amino acid sequence of SEQ ID NO: 1531. In embodiments, the peptide includes, or is, the amino acid sequence of SEQ ID NO: 1532. In embodiments, the peptide includes, or is, the amino acid sequence of SEQ ID NO: 1533. In embodiments, the peptide includes, or is, the amino acid sequence of SEQ ID NO: 1534. In embodiments, the peptide includes, or is, the amino acid sequence of SEQ ID NO: 1535. In embodiments, the peptide includes, or is, the amino acid sequence of SEQ ID NO: 1536. In embodiments, the peptide includes, or is, the amino acid sequence of SEQ ID NO: 1537. In embodiments, the peptide includes, or is, the amino acid sequence of SEQ ID NO: 1538. In embodiments, the peptide includes, or is, the amino acid sequence of SEQ ID NO: 1539. In embodiments, the peptide includes, or is, the amino acid sequence of SEQ ID NO: 1540. In embodiments, the peptide includes, or is, the amino acid sequence of SEQ ID NO: 1541. In embodiments, the peptide includes, or is, the amino acid sequence of SEQ ID NO: 1542. In embodiments, the peptide includes, or is, the amino acid sequence of SEQ ID NO: 1543. In embodiments, the peptide includes, or is, the amino acid sequence of SEQ ID NO: 1544. In embodiments, the peptide includes, or is, the amino acid sequence of SEQ ID NO: 1545. In embodiments, the peptide includes, or is, the amino acid sequence of SEQ ID NO: 1546. In embodiments, the peptide includes, or is, the amino acid sequence of SEQ ID NO: 1547. In embodiments, the peptide includes, or is, the amino acid sequence of SEQ ID NO: 1548. In embodiments, the peptide includes, or is, the amino acid sequence of SEQ ID NO: 1549. In embodiments, the peptide includes, or is, the amino acid sequence of SEQ ID NO: 1550.

In embodiments, the peptide includes, or is, the amino acid sequence of SEQ ID NO: 1551. In embodiments, the peptide includes, or is, the amino acid sequence of SEQ ID NO: 1552. In embodiments, the peptide includes, or is, the amino acid sequence of SEQ ID NO: 1553. In embodiments, the peptide includes, or is, the amino acid sequence of SEQ ID NO: 1554. In embodiments, the peptide includes, or is, the amino acid sequence of SEQ ID NO: 1555. In embodiments, the peptide includes, or is, the amino acid sequence of SEQ ID NO: 1556. In embodiments, the peptide includes, or is, the amino acid sequence of SEQ ID NO: 1557. In embodiments, the peptide includes, or is, the amino acid sequence of SEQ ID NO: 1558. In embodiments, the peptide includes, or is, the amino acid sequence of SEQ ID NO: 1559. In embodiments, the peptide includes, or is, the amino acid sequence of SEQ ID NO: 1560. In embodiments, the peptide includes, or is, the amino acid sequence of SEQ ID NO: 1561. In embodiments, the peptide includes, or is, the amino acid sequence of SEQ ID NO: 1562. In embodiments, the peptide includes, or is, the amino acid sequence of SEQ ID NO: 1563. In embodiments, the peptide includes, or is, the amino acid sequence of SEQ ID NO: 1564. In embodiments, the peptide includes, or is, the amino acid sequence of SEQ ID NO: 1565. In embodiments, the peptide includes, or is, the amino acid sequence of SEQ ID NO: 1566. In embodiments, the peptide includes, or is, the amino acid sequence of SEQ ID NO: 1567. In embodiments, the peptide includes, or is, the amino acid sequence of SEQ ID NO: 1568. In embodiments, the peptide includes, or is, the amino acid sequence of SEQ ID NO: 1569. In embodiments, the peptide includes, or is, the amino acid sequence of SEQ ID NO: 1570. In embodiments, the peptide includes, or is, the amino acid sequence of SEQ ID NO: 1571. In embodiments, the peptide includes, or is, the amino acid sequence of SEQ ID NO: 1572. In embodiments, the peptide includes, or is, the amino acid sequence of SEQ ID NO: 1573. In embodiments, the peptide includes, or is, the amino acid sequence of SEQ ID NO: 1574. In embodiments, the peptide includes, or is, the amino acid sequence of SEQ ID NO: 1575.

In embodiments, the peptide includes, or is, the amino acid sequence of SEQ ID NO: 1576. In embodiments, the peptide includes, or is, the amino acid sequence of SEQ ID NO: 1577. In embodiments, the peptide includes, or is, the amino acid sequence of SEQ ID NO: 1578. In embodiments, the peptide includes, or is, the amino acid sequence of SEQ ID NO: 1579. In embodiments, the peptide includes, or is, the amino acid sequence of SEQ ID NO: 1580. In embodiments, the peptide includes, or is, the amino acid sequence of SEQ ID NO: 1581. In embodiments, the peptide includes, or is, the amino acid sequence of SEQ ID NO: 1582. In embodiments, the peptide includes, or is, the amino acid sequence of SEQ ID NO: 1583. In embodiments, the peptide includes, or is, the amino acid sequence of SEQ ID NO: 1584. In embodiments, the peptide includes, or is, the amino acid sequence of SEQ ID NO: 1585. In embodiments, the peptide includes, or is, the amino acid sequence of SEQ ID NO: 1586. In embodiments, the peptide includes, or is, the amino acid sequence of SEQ ID NO: 1587. In embodiments, the peptide includes, or is, the amino acid sequence of SEQ ID NO: 1588. In embodiments, the peptide includes, or is, the amino acid sequence of SEQ ID NO: 1589. In embodiments, the peptide includes, or is, the amino acid sequence of SEQ ID NO: 1590. In embodiments, the peptide includes, or is, the amino acid sequence of SEQ ID NO: 1591. In embodiments, the peptide includes, or is, the amino acid sequence of SEQ ID NO: 1592. In embodiments, the peptide includes, or is, the amino acid sequence of SEQ ID NO: 1593. In embodiments, the peptide includes, or is, the amino acid sequence of SEQ ID NO: 1594. In embodiments, the peptide includes, or is, the amino acid sequence of SEQ ID NO: 1595. In embodiments, the peptide includes, or is, the amino acid sequence of SEQ ID NO: 1596. In embodiments, the peptide includes, or is, the amino acid sequence of SEQ ID NO: 1597. In embodiments, the peptide includes, or is, the amino acid sequence of SEQ ID NO: 1598. In embodiments, the peptide includes, or is, the amino acid sequence of SEQ ID NO: 1599.

In embodiments, the peptide includes, or is, the amino acid sequence of SEQ ID NO: 1600. In embodiments, the peptide includes, or is, the amino acid sequence of SEQ ID NO: 1601. In embodiments, the peptide includes, or is, the amino acid sequence of SEQ ID NO: 1602. In embodiments, the peptide includes, or is, the amino acid sequence of SEQ ID NO: 1603. In embodiments, the peptide includes, or is, the amino acid sequence of SEQ ID NO: 1604. In embodiments, the peptide includes, or is, the amino acid sequence of SEQ ID NO: 1605. In embodiments, the peptide includes, or is, the amino acid sequence of SEQ ID NO: 1606. In embodiments, the peptide includes, or is, the amino acid sequence of SEQ ID NO: 1607. In embodiments, the peptide includes, or is, the amino acid sequence of SEQ ID NO: 1608. In embodiments, the peptide includes, or is, the amino acid sequence of SEQ ID NO: 1609. In embodiments, the peptide includes, or is, the amino acid sequence of SEQ ID NO: 1610. In embodiments, the peptide includes, or is, the amino acid sequence of SEQ ID NO: 1611. In embodiments, the peptide includes, or is, the amino acid sequence of SEQ ID NO: 1612. In embodiments, the peptide includes, or is, the amino acid sequence of SEQ ID NO: 1613. In embodiments, the peptide includes, or is, the amino acid sequence of SEQ ID NO: 1614. In embodiments, the peptide includes, or is, the amino acid sequence of SEQ ID NO: 1615. In embodiments, the peptide includes, or is, the amino acid sequence of SEQ ID NO: 1616. In embodiments, the peptide includes, or is, the amino acid sequence of SEQ ID NO: 1617. In embodiments, the peptide includes, or is, the amino acid sequence of SEQ ID NO: 1618. In embodiments, the peptide includes, or is, the amino acid sequence of SEQ ID NO: 1619. In embodiments, the peptide includes, or is, the amino acid sequence of SEQ ID NO: 1620. In embodiments, the peptide includes, or is, the amino acid sequence of SEQ ID NO: 1621. In embodiments, the peptide includes, or is, the amino acid sequence of SEQ ID NO: 1622. In embodiments, the peptide includes, or is, the amino acid sequence of SEQ ID NO: 1623. In embodiments, the peptide includes, or is, the amino acid sequence of SEQ ID NO: 1624. In embodiments, the peptide includes, or is, the amino acid sequence of SEQ ID NO: 1625.

In embodiments, the peptide includes, or is, the amino acid sequence of SEQ ID NO: 1626. In embodiments, the peptide includes, or is, the amino acid sequence of SEQ ID NO: 1627. In embodiments, the peptide includes, or is, the amino acid sequence of SEQ ID NO: 1628. In embodiments, the peptide includes, or is, the amino acid sequence of SEQ ID NO: 1629. In embodiments, the peptide includes, or is, the amino acid sequence of SEQ ID NO: 1630. In embodiments, the peptide includes, or is, the amino acid sequence of SEQ ID NO: 1631. In embodiments, the peptide includes, or is, the amino acid sequence of SEQ ID NO: 1632. In embodiments, the peptide includes, or is, the amino acid sequence of SEQ ID NO: 1633. In embodiments, the peptide includes, or is, the amino acid sequence of SEQ ID NO: 1634. In embodiments, the peptide includes, or is, the amino acid sequence of SEQ ID NO: 1635. In embodiments, the peptide includes, or is, the amino acid sequence of SEQ ID NO: 1636. In embodiments, the peptide includes, or is, the amino acid sequence of SEQ ID NO: 1637. In embodiments, the peptide includes, or is, the amino acid sequence of SEQ ID NO: 1638. In embodiments, the peptide includes, or is, the amino acid sequence of SEQ ID NO: 1639. In embodiments, the peptide includes, or is, the amino acid sequence of SEQ ID NO: 1640. In embodiments, the peptide includes, or is, the amino acid sequence of SEQ ID NO: 1641. In embodiments, the peptide includes, or is, the amino acid sequence of SEQ ID NO: 1642. In embodiments, the peptide includes, or is, the amino acid sequence of SEQ ID NO: 1643. In embodiments, the peptide includes, or is, the amino acid sequence of SEQ ID NO: 1644. In embodiments, the peptide includes, or is, the amino acid sequence of SEQ ID NO: 1645. In embodiments, the peptide includes, or is, the amino acid sequence of SEQ ID NO: 1646. In embodiments, the peptide includes, or is, the amino acid sequence of SEQ ID NO: 1647. In embodiments, the peptide includes, or is, the amino acid sequence of SEQ ID NO: 1648. In embodiments, the peptide includes, or is, the amino acid sequence of SEQ ID NO: 1649. In embodiments, the peptide includes, or is, the amino acid sequence of SEQ ID NO: 1650.

In embodiments, the peptide includes, or is, the amino acid sequence of SEQ ID NO: 1651. In embodiments, the peptide includes, or is, the amino acid sequence of SEQ ID NO: 1652. In embodiments, the peptide includes, or is, the amino acid sequence of SEQ ID NO: 1653. In embodiments, the peptide includes, or is, the amino acid sequence of SEQ ID NO: 1654. In embodiments, the peptide includes, or is, the amino acid sequence of SEQ ID NO: 1655. In embodiments, the peptide includes, or is, the amino acid sequence of SEQ ID NO: 1656. In embodiments, the peptide includes, or is, the amino acid sequence of SEQ ID NO: 1657. In embodiments, the peptide includes, or is, the amino acid sequence of SEQ ID NO: 1658. In embodiments, the peptide includes, or is, the amino acid sequence of SEQ ID NO: 1659. In embodiments, the peptide includes, or is, the amino acid sequence of SEQ ID NO: 1660. In embodiments, the peptide includes, or is, the amino acid sequence of SEQ ID NO: 1661. In embodiments, the peptide includes, or is, the amino acid sequence of SEQ ID NO: 1662. In embodiments, the peptide includes, or is, the amino acid sequence of SEQ ID NO: 1663. In embodiments, the peptide includes, or is, the amino acid sequence of SEQ ID NO: 1664. In embodiments, the peptide includes, or is, the amino acid sequence of SEQ ID NO: 1665. In embodiments, the peptide includes, or is, the amino acid sequence of SEQ ID NO: 1666. In embodiments, the peptide includes, or is, the amino acid sequence of SEQ ID NO: 1667. In embodiments, the peptide includes, or is, the amino acid sequence of SEQ ID NO: 1668. In embodiments, the peptide includes, or is, the amino acid sequence of SEQ ID NO: 1669. In embodiments, the peptide includes, or is, the amino acid sequence of SEQ ID NO: 1670. In embodiments, the peptide includes, or is, the amino acid sequence of SEQ ID NO: 1671. In embodiments, the peptide includes, or is, the amino acid sequence of SEQ ID NO: 1672. In embodiments, the peptide includes, or is, the amino acid sequence of SEQ ID NO: 1673. In embodiments, the peptide includes, or is, the amino acid sequence of SEQ ID NO: 1674. In embodiments, the peptide includes, or is, the amino acid sequence of SEQ ID NO: 1675.

In embodiments, the peptide includes, or is, the amino acid sequence of SEQ ID NO: 1676. In embodiments, the peptide includes, or is, the amino acid sequence of SEQ ID NO: 1677. In embodiments, the peptide includes, or is, the amino acid sequence of SEQ ID NO: 1678. In embodiments, the peptide includes, or is, the amino acid sequence of SEQ ID NO: 1679. In embodiments, the peptide includes, or is, the amino acid sequence of SEQ ID NO: 1680. In embodiments, the peptide includes, or is, the amino acid sequence of SEQ ID NO: 1681. In embodiments, the peptide includes, or is, the amino acid sequence of SEQ ID NO: 1682. In embodiments, the peptide includes, or is, the amino acid sequence of SEQ ID NO: 1683. In embodiments, the peptide includes, or is, the amino acid sequence of SEQ ID NO: 1684. In embodiments, the peptide includes, or is, the amino acid sequence of SEQ ID NO: 1685. In embodiments, the peptide includes, or is, the amino acid sequence of SEQ ID NO: 1686. In embodiments, the peptide includes, or is, the amino acid sequence of SEQ ID NO: 1687. In embodiments, the peptide includes, or is, the amino acid sequence of SEQ ID NO: 1688. In embodiments, the peptide includes, or is, the amino acid sequence of SEQ ID NO: 1689. In embodiments, the peptide includes, or is, the amino acid sequence of SEQ ID NO: 1690. In embodiments, the peptide includes, or is, the amino acid sequence of SEQ ID NO: 1691. In embodiments, the peptide includes, or is, the amino acid sequence of SEQ ID NO: 1692. In embodiments, the peptide includes, or is, the amino acid sequence of SEQ ID NO: 1693. In embodiments, the peptide includes, or is, the amino acid sequence of SEQ ID NO: 1694. In embodiments, the peptide includes, or is, the amino acid sequence of SEQ ID NO: 1695. In embodiments, the peptide includes, or is, the amino acid sequence of SEQ ID NO: 1696. In embodiments, the peptide includes, or is, the amino acid sequence of SEQ ID NO: 1697. In embodiments, the peptide includes, or is, the amino acid sequence of SEQ ID NO: 1698. In embodiments, the peptide includes, or is, the amino acid sequence of SEQ ID NO: 1699. In embodiments, the peptide includes, or is, the amino acid sequence of SEQ ID NO: 1700.

In embodiments, the peptide includes, or is, the amino acid sequence of SEQ ID NO: 1701. In embodiments, the peptide includes, or is, the amino acid sequence of SEQ ID NO: 1702. In embodiments, the peptide includes, or is, the amino acid sequence of SEQ ID NO: 1703. In embodiments, the peptide includes, or is, the amino acid sequence of SEQ ID NO: 1704. In embodiments, the peptide includes, or is, the amino acid sequence of SEQ ID NO: 1705. In embodiments, the peptide includes, or is, the amino acid sequence of SEQ ID NO: 1706. In embodiments, the peptide includes, or is, the amino acid sequence of SEQ ID NO: 1707. In embodiments, the peptide includes, or is, the amino acid sequence of SEQ ID NO: 1708. In embodiments, the peptide includes, or is, the amino acid sequence of SEQ ID NO: 1709. In embodiments, the peptide includes, or is, the amino acid sequence of SEQ ID NO: 1710. In embodiments, the peptide includes, or is, the amino acid sequence of SEQ ID NO: 1711. In embodiments, the peptide includes, or is, the amino acid sequence of SEQ ID NO: 1712. In embodiments, the peptide includes, or is, the amino acid sequence of SEQ ID NO: 1713. In embodiments, the peptide includes, or is, the amino acid sequence of SEQ ID NO: 1714. In embodiments, the peptide includes, or is, the amino acid sequence of SEQ ID NO: 1715. In embodiments, the peptide includes, or is, the amino acid sequence of SEQ ID NO: 1716. In embodiments, the peptide includes, or is, the amino acid sequence of SEQ ID NO: 1717. In embodiments, the peptide includes, or is, the amino acid sequence of SEQ ID NO: 1718. In embodiments, the peptide includes, or is, the amino acid sequence of SEQ ID NO: 1719. In embodiments, the peptide includes, or is, the amino acid sequence of SEQ ID NO: 1720. In embodiments, the peptide includes, or is, the amino acid sequence of SEQ ID NO: 1721. In embodiments, the peptide includes, or is, the amino acid sequence of SEQ ID NO: 1722. In embodiments, the peptide includes, or is, the amino acid sequence of SEQ ID NO: 1723. In embodiments, the peptide includes, or is, the amino acid sequence of SEQ ID NO: 1724. In embodiments, the peptide includes, or is, the amino acid sequence of SEQ ID NO: 1725.

In embodiments, the peptide includes, or is, the amino acid sequence of SEQ ID NO: 1726. In embodiments, the peptide includes, or is, the amino acid sequence of SEQ ID NO: 1727. In embodiments, the peptide includes, or is, the amino acid sequence of SEQ ID NO: 1728. In embodiments, the peptide includes, or is, the amino acid sequence of SEQ ID NO: 1729. In embodiments, the peptide includes, or is, the amino acid sequence of SEQ ID NO: 1730. In embodiments, the peptide includes, or is, the amino acid sequence of SEQ ID NO: 1731. In embodiments, the peptide includes, or is, the amino acid sequence of SEQ ID NO: 1732. In embodiments, the peptide includes, or is, the amino acid sequence of SEQ ID NO: 1733. In embodiments, the peptide includes, or is, the amino acid sequence of SEQ ID NO: 1734. In embodiments, the peptide includes, or is, the amino acid sequence of SEQ ID NO: 1735. In embodiments, the peptide includes, or is, the amino acid sequence of SEQ ID NO: 1736. In embodiments, the peptide includes, or is, the amino acid sequence of SEQ ID NO: 1737. In embodiments, the peptide includes, or is, the amino acid sequence of SEQ ID NO: 1738. In embodiments, the peptide includes, or is, the amino acid sequence of SEQ ID NO: 1739. In embodiments, the peptide includes, or is, the amino acid sequence of SEQ ID NO: 1740. In embodiments, the peptide includes, or is, the amino acid sequence of SEQ ID NO: 1741. In embodiments, the peptide includes, or is, the amino acid sequence of SEQ ID NO: 1742. In embodiments, the peptide includes, or is, the amino acid sequence of SEQ ID NO: 1743. In embodiments, the peptide includes, or is, the amino acid sequence of SEQ ID NO: 1744. In embodiments, the peptide includes, or is, the amino acid sequence of SEQ ID NO: 1745. In embodiments, the peptide includes, or is, the amino acid sequence of SEQ ID NO: 1746. In embodiments, the peptide includes, or is, the amino acid sequence of SEQ ID NO: 1747. In embodiments, the peptide includes, or is, the amino acid sequence of SEQ ID NO: 1748. In embodiments, the peptide includes, or is, the amino acid sequence of SEQ ID NO: 1749. In embodiments, the peptide includes, or is, the amino acid sequence of SEQ ID NO: 1750.

In embodiments, the peptide includes, or is, the amino acid sequence of SEQ ID NO: 1751. In embodiments, the peptide includes, or is, the amino acid sequence of SEQ ID NO: 1752. In embodiments, the peptide includes, or is, the amino acid sequence of SEQ ID NO: 1753. In embodiments, the peptide includes, or is, the amino acid sequence of SEQ ID NO: 1754. In embodiments, the peptide includes, or is, the amino acid sequence of SEQ ID NO: 1755. In embodiments, the peptide includes, or is, the amino acid sequence of SEQ ID NO: 1756. In embodiments, the peptide includes, or is, the amino acid sequence of SEQ ID NO: 1757. In embodiments, the peptide includes, or is, the amino acid sequence of SEQ ID NO: 1758. In embodiments, the peptide includes, or is, the amino acid sequence of SEQ ID NO: 1759. In embodiments, the peptide includes, or is, the amino acid sequence of SEQ ID NO: 1760. In embodiments, the peptide includes, or is, the amino acid sequence of SEQ ID NO: 1761. In embodiments, the peptide includes, or is, the amino acid sequence of SEQ ID NO: 1762. In embodiments, the peptide includes, or is, the amino acid sequence of SEQ ID NO: 1763. In embodiments, the peptide includes, or is, the amino acid sequence of SEQ ID NO: 1764. In embodiments, the peptide includes, or is, the amino acid sequence of SEQ ID NO: 1765. In embodiments, the peptide includes, or is, the amino acid sequence of SEQ ID NO: 1766. In embodiments, the peptide includes, or is, the amino acid sequence of SEQ ID NO: 1767. In embodiments, the peptide includes, or is, the amino acid sequence of SEQ ID NO: 1768. In embodiments, the peptide includes, or is, the amino acid sequence of SEQ ID NO: 1769. In embodiments, the peptide includes, or is, the amino acid sequence of SEQ ID NO: 1770. In embodiments, the peptide includes, or is, the amino acid sequence of SEQ ID NO: 1771. In embodiments, the peptide includes, or is, the amino acid sequence of SEQ ID NO: 1772. In embodiments, the peptide includes, or is, the amino acid sequence of SEQ ID NO: 1773. In embodiments, the peptide includes, or is, the amino acid sequence of SEQ ID NO: 1774. In embodiments, the peptide includes, or is, the amino acid sequence of SEQ ID NO: 1775.

In embodiments, the peptide includes, or is, the amino acid sequence of SEQ ID NO: 1776. In embodiments, the peptide includes, or is, the amino acid sequence of SEQ ID NO: 1777. In embodiments, the peptide includes, or is, the amino acid sequence of SEQ ID NO: 1778. In embodiments, the peptide includes, or is, the amino acid sequence of SEQ ID NO: 1779. In embodiments, the peptide includes, or is, the amino acid sequence of SEQ ID NO: 1780. In embodiments, the peptide includes, or is, the amino acid sequence of SEQ ID NO: 1781. In embodiments, the peptide includes, or is, the amino acid sequence of SEQ ID NO: 1782. In embodiments, the peptide includes, or is, the amino acid sequence of SEQ ID NO: 1783. In embodiments, the peptide includes, or is, the amino acid sequence of SEQ ID NO: 1784. In embodiments, the peptide includes, or is, the amino acid sequence of SEQ ID NO: 1785. In embodiments, the peptide includes, or is, the amino acid sequence of SEQ ID NO: 1786. In embodiments, the peptide includes, or is, the amino acid sequence of SEQ ID NO: 1787. In embodiments, the peptide includes, or is, the amino acid sequence of SEQ ID NO: 1788. In embodiments, the peptide includes, or is, the amino acid sequence of SEQ ID NO: 1789. In embodiments, the peptide includes, or is, the amino acid sequence of SEQ ID NO: 1790. In embodiments, the peptide includes, or is, the amino acid sequence of SEQ ID NO: 1791. In embodiments, the peptide includes, or is, the amino acid sequence of SEQ ID NO: 1792. In embodiments, the peptide includes, or is, the amino acid sequence of SEQ ID NO: 1793. In embodiments, the peptide includes, or is, the amino acid sequence of SEQ ID NO: 1794. In embodiments, the peptide includes, or is, the amino acid sequence of SEQ ID NO: 1795. In embodiments, the peptide includes, or is, the amino acid sequence of SEQ ID NO: 1796. In embodiments, the peptide includes, or is, the amino acid sequence of SEQ ID NO: 1797. In embodiments, the peptide includes, or is, the amino acid sequence of SEQ ID NO: 1798. In embodiments, the peptide includes, or is, the amino acid sequence of SEQ ID NO: 1799. In embodiments, the peptide includes, or is, the amino acid sequence of SEQ ID NO: 1800.

In embodiments, the peptide includes, or is, the amino acid sequence of SEQ ID NO: 1801. In embodiments, the peptide includes, or is, the amino acid sequence of SEQ ID NO: 1802. In embodiments, the peptide includes, or is, the amino acid sequence of SEQ ID NO: 1803. In embodiments, the peptide includes, or is, the amino acid sequence of SEQ ID NO: 1804. In embodiments, the peptide includes, or is, the amino acid sequence of SEQ ID NO: 1805. In embodiments, the peptide includes, or is, the amino acid sequence of SEQ ID NO: 1806. In embodiments, the peptide includes, or is, the amino acid sequence of SEQ ID NO: 1807. In embodiments, the peptide includes, or is, the amino acid sequence of SEQ ID NO: 1808. In embodiments, the peptide includes, or is, the amino acid sequence of SEQ ID NO: 1809. In embodiments, the peptide includes, or is, the amino acid sequence of SEQ ID NO: 1810. In embodiments, the peptide includes, or is, the amino acid sequence of SEQ ID NO: 1811. In embodiments, the peptide includes, or is, the amino acid sequence of SEQ ID NO: 1812. In embodiments, the peptide includes, or is, the amino acid sequence of SEQ ID NO: 1813. In embodiments, the peptide includes, or is, the amino acid sequence of SEQ ID NO: 1814. In embodiments, the peptide includes, or is, the amino acid sequence of SEQ ID NO: 1815. In embodiments, the peptide includes, or is, the amino acid sequence of SEQ ID NO: 1816. In embodiments, the peptide includes, or is, the amino acid sequence of SEQ ID NO: 1817. In embodiments, the peptide includes, or is, the amino acid sequence of SEQ ID NO: 1818. In embodiments, the peptide includes, or is, the amino acid sequence of SEQ ID NO: 1819. In embodiments, the peptide includes, or is, the amino acid sequence of SEQ ID NO: 1820. In embodiments, the peptide includes, or is, the amino acid sequence of SEQ ID NO: 1821. In embodiments, the peptide includes, or is, the amino acid sequence of SEQ ID NO: 1822. In embodiments, the peptide includes, or is, the amino acid sequence of SEQ ID NO: 1823. In embodiments, the peptide includes, or is, the amino acid sequence of SEQ ID NO: 1824. In embodiments, the peptide includes, or is, the amino acid sequence of SEQ ID NO: 1825.

In embodiments, the peptide includes, or is, the amino acid sequence of SEQ ID NO: 1826. In embodiments, the peptide includes, or is, the amino acid sequence of SEQ ID NO: 1827. In embodiments, the peptide includes, or is, the amino acid sequence of SEQ ID NO: 1828. In embodiments, the peptide includes, or is, the amino acid sequence of SEQ ID NO: 1829. In embodiments, the peptide includes, or is, the amino acid sequence of SEQ ID NO: 1830. In embodiments, the peptide includes, or is, the amino acid sequence of SEQ ID NO: 1831. In embodiments, the peptide includes, or is, the amino acid sequence of SEQ ID NO: 1832. In embodiments, the peptide includes, or is, the amino acid sequence of SEQ ID NO: 1833. In embodiments, the peptide includes, or is, the amino acid sequence of SEQ ID NO: 1834. In embodiments, the peptide includes, or is, the amino acid sequence of SEQ ID NO: 1835. In embodiments, the peptide includes, or is, the amino acid sequence of SEQ ID NO: 1836. In embodiments, the peptide includes, or is, the amino acid sequence of SEQ ID NO: 1837. In embodiments, the peptide includes, or is, the amino acid sequence of SEQ ID NO: 1838. In embodiments, the peptide includes, or is, the amino acid sequence of SEQ ID NO: 1839. In embodiments, the peptide includes, or is, the amino acid sequence of SEQ ID NO: 1840. In embodiments, the peptide includes, or is, the amino acid sequence of SEQ ID NO: 1841. In embodiments, the peptide includes, or is, the amino acid sequence of SEQ ID NO: 1842. In embodiments, the peptide includes, or is, the amino acid sequence of SEQ ID NO: 1843. In embodiments, the peptide includes, or is, the amino acid sequence of SEQ ID NO: 1844. In embodiments, the peptide includes, or is, the amino acid sequence of SEQ ID NO: 1845. In embodiments, the peptide includes, or is, the amino acid sequence of SEQ ID NO: 1846. In embodiments, the peptide includes, or is, the amino acid sequence of SEQ ID NO: 1847. In embodiments, the peptide includes, or is, the amino acid sequence of SEQ ID NO: 1848. In embodiments, the peptide includes, or is, the amino acid sequence of SEQ ID NO: 1849. In embodiments, the peptide includes, or is, the amino acid sequence of SEQ ID NO: 1850.

In embodiments, the peptide includes, or is, the amino acid sequence of SEQ ID NO: 1851. In embodiments, the peptide includes, or is, the amino acid sequence of SEQ ID NO: 1852. In embodiments, the peptide includes, or is, the amino acid sequence of SEQ ID NO: 1853. In embodiments, the peptide includes, or is, the amino acid sequence of SEQ ID NO: 1854. In embodiments, the peptide includes, or is, the amino acid sequence of SEQ ID NO: 1855. In embodiments, the peptide includes, or is, the amino acid sequence of SEQ ID NO: 1856. In embodiments, the peptide includes, or is, the amino acid sequence of SEQ ID NO: 1857. In embodiments, the peptide includes, or is, the amino acid sequence of SEQ ID NO: 1858. In embodiments, the peptide includes, or is, the amino acid sequence of SEQ ID NO: 1859. In embodiments, the peptide includes, or is, the amino acid sequence of SEQ ID NO: 1860. In embodiments, the peptide includes, or is, the amino acid sequence of SEQ ID NO: 1861. In embodiments, the peptide includes, or is, the amino acid sequence of SEQ ID NO: 1862. In embodiments, the peptide includes, or is, the amino acid sequence of SEQ ID NO: 1863. In embodiments, the peptide includes, or is, the amino acid sequence of SEQ ID NO: 1864. In embodiments, the peptide includes, or is, the amino acid sequence of SEQ ID NO: 1865. In embodiments, the peptide includes, or is, the amino acid sequence of SEQ ID NO: 1866. In embodiments, the peptide includes, or is, the amino acid sequence of SEQ ID NO: 1867. In embodiments, the peptide includes, or is, the amino acid sequence of SEQ ID NO: 1868. In embodiments, the peptide includes, or is, the amino acid sequence of SEQ ID NO: 1869. In embodiments, the peptide includes, or is, the amino acid sequence of SEQ ID NO: 1870. In embodiments, the peptide includes, or is, the amino acid sequence of SEQ ID NO: 1871. In embodiments, the peptide includes, or is, the amino acid sequence of SEQ ID NO: 1872. In embodiments, the peptide includes, or is, the amino acid sequence of SEQ ID NO: 1873. In embodiments, the peptide includes, or is, the amino acid sequence of SEQ ID NO: 1874. In embodiments, the peptide includes, or is, the amino acid sequence of SEQ ID NO: 1875.

In embodiments, the peptide includes, or is, the amino acid sequence of SEQ ID NO: 1876. In embodiments, the peptide includes, or is, the amino acid sequence of SEQ ID NO: 1877. In embodiments, the peptide includes, or is, the amino acid sequence of SEQ ID NO: 1878. In embodiments, the peptide includes, or is, the amino acid sequence of SEQ ID NO: 1879. In embodiments, the peptide includes, or is, the amino acid sequence of SEQ ID NO: 1880. In embodiments, the peptide includes, or is, the amino acid sequence of SEQ ID NO: 1881. In embodiments, the peptide includes, or is, the amino acid sequence of SEQ ID NO: 1882. In embodiments, the peptide includes, or is, the amino acid sequence of SEQ ID NO: 1883. In embodiments, the peptide includes, or is, the amino acid sequence of SEQ ID NO: 1884. In embodiments, the peptide includes, or is, the amino acid sequence of SEQ ID NO: 1885. In embodiments, the peptide includes, or is, the amino acid sequence of SEQ ID NO: 1886. In embodiments, the peptide includes, or is, the amino acid sequence of SEQ ID NO: 1887. In embodiments, the peptide includes, or is, the amino acid sequence of SEQ ID NO: 1888. In embodiments, the peptide includes, or is, the amino acid sequence of SEQ ID NO: 1889. In embodiments, the peptide includes, or is, the amino acid sequence of SEQ ID NO: 1890. In embodiments, the peptide includes, or is, the amino acid sequence of SEQ ID NO: 1891. In embodiments, the peptide includes, or is, the amino acid sequence of SEQ ID NO: 1892. In embodiments, the peptide includes, or is, the amino acid sequence of SEQ ID NO: 1893. In embodiments, the peptide includes, or is, the amino acid sequence of SEQ ID NO: 1894. In embodiments, the peptide includes, or is, the amino acid sequence of SEQ ID NO: 1895. In embodiments, the peptide includes, or is, the amino acid sequence of SEQ ID NO: 1896. In embodiments, the peptide includes, or is, the amino acid sequence of SEQ ID NO: 1897. In embodiments, the peptide includes, or is, the amino acid sequence of SEQ ID NO: 1898. In embodiments, the peptide includes, or is, the amino acid sequence of SEQ ID NO: 1899. In embodiments, the peptide includes, or is, the amino acid sequence of SEQ ID NO: 1900.

In embodiments, the peptide includes, or is, the amino acid sequence of SEQ ID NO: 1901. In embodiments, the peptide includes, or is, the amino acid sequence of SEQ ID NO: 1902. In embodiments, the peptide includes, or is, the amino acid sequence of SEQ ID NO: 1903. In embodiments, the peptide includes, or is, the amino acid sequence of SEQ ID NO: 1904. In embodiments, the peptide includes, or is, the amino acid sequence of SEQ ID NO: 1905. In embodiments, the peptide includes, or is, the amino acid sequence of SEQ ID NO: 1906. In embodiments, the peptide includes, or is, the amino acid sequence of SEQ ID NO: 1907. In embodiments, the peptide includes, or is, the amino acid sequence of SEQ ID NO: 1908. In embodiments, the peptide includes, or is, the amino acid sequence of SEQ ID NO: 1909. In embodiments, the peptide includes, or is, the amino acid sequence of SEQ ID NO: 1910. In embodiments, the peptide includes, or is, the amino acid sequence of SEQ ID NO: 1911. In embodiments, the peptide includes, or is, the amino acid sequence of SEQ ID NO: 1912. In embodiments, the peptide includes, or is, the amino acid sequence of SEQ ID NO: 1913. In embodiments, the peptide includes, or is, the amino acid sequence of SEQ ID NO: 1914. In embodiments, the peptide includes, or is, the amino acid sequence of SEQ ID NO: 1915. In embodiments, the peptide includes, or is, the amino acid sequence of SEQ ID NO: 1916. In embodiments, the peptide includes, or is, the amino acid sequence of SEQ ID NO: 1917. In embodiments, the peptide includes, or is, the amino acid sequence of SEQ ID NO: 1918. In embodiments, the peptide includes, or is, the amino acid sequence of SEQ ID NO: 1919. In embodiments, the peptide includes, or is, the amino acid sequence of SEQ ID NO: 1920. In embodiments, the peptide includes, or is, the amino acid sequence of SEQ ID NO: 1921. In embodiments, the peptide includes, or is, the amino acid sequence of SEQ ID NO: 1922. In embodiments, the peptide includes, or is, the amino acid sequence of SEQ ID NO: 1923. In embodiments, the peptide includes, or is, the amino acid sequence of SEQ ID NO: 1924. In embodiments, the peptide includes, or is, the amino acid sequence of SEQ ID NO: 1925.

In embodiments, the peptide includes, or is, the amino acid sequence of SEQ ID NO: 1926. In embodiments, the peptide includes, or is, the amino acid sequence of SEQ ID NO: 1927. In embodiments, the peptide includes, or is, the amino acid sequence of SEQ ID NO: 1928. In embodiments, the peptide includes, or is, the amino acid sequence of SEQ ID NO: 1929. In embodiments, the peptide includes, or is, the amino acid sequence of SEQ ID NO: 1930. In embodiments, the peptide includes, or is, the amino acid sequence of SEQ ID NO: 1931. In embodiments, the peptide includes, or is, the amino acid sequence of SEQ ID NO: 1932. In embodiments, the peptide includes, or is, the amino acid sequence of SEQ ID NO: 1933. In embodiments, the peptide includes, or is, the amino acid sequence of SEQ ID NO: 1934. In embodiments, the peptide includes, or is, the amino acid sequence of SEQ ID NO: 1935. In embodiments, the peptide includes, or is, the amino acid sequence of SEQ ID NO: 1936. In embodiments, the peptide includes, or is, the amino acid sequence of SEQ ID NO: 1937. In embodiments, the peptide includes, or is, the amino acid sequence of SEQ ID NO: 1938. In embodiments, the peptide includes, or is, the amino acid sequence of SEQ ID NO: 1939. In embodiments, the peptide includes, or is, the amino acid sequence of SEQ ID NO: 1940. In embodiments, the peptide includes, or is, the amino acid sequence of SEQ ID NO: 1941. In embodiments, the peptide includes, or is, the amino acid sequence of SEQ ID NO: 1942. In embodiments, the peptide includes, or is, the amino acid sequence of SEQ ID NO: 1943. In embodiments, the peptide includes, or is, the amino acid sequence of SEQ ID NO: 1944. In embodiments, the peptide includes, or is, the amino acid sequence of SEQ ID NO: 1945. In embodiments, the peptide includes, or is, the amino acid sequence of SEQ ID NO: 1946. In embodiments, the peptide includes, or is, the amino acid sequence of SEQ ID NO: 1947. In embodiments, the peptide includes, or is, the amino acid sequence of SEQ ID NO: 1948. In embodiments, the peptide includes, or is, the amino acid sequence of SEQ ID NO: 1949. In embodiments, the peptide includes, or is, the amino acid sequence of SEQ ID NO: 1950.

In embodiments, the peptide includes, or is, the amino acid sequence of SEQ ID NO: 1951. In embodiments, the peptide includes, or is, the amino acid sequence of SEQ ID NO: 1952. In embodiments, the peptide includes, or is, the amino acid sequence of SEQ ID NO: 1953. In embodiments, the peptide includes, or is, the amino acid sequence of SEQ ID NO: 1954. In embodiments, the peptide includes, or is, the amino acid sequence of SEQ ID NO: 1955. In embodiments, the peptide includes, or is, the amino acid sequence of SEQ ID NO: 1956. In embodiments, the peptide includes, or is, the amino acid sequence of SEQ ID NO: 1957. In embodiments, the peptide includes, or is, the amino acid sequence of SEQ ID NO: 1958. In embodiments, the peptide includes, or is, the amino acid sequence of SEQ ID NO: 1959. In embodiments, the peptide includes, or is, the amino acid sequence of SEQ ID NO: 1960. In embodiments, the peptide includes, or is, the amino acid sequence of SEQ ID NO: 1961. In embodiments, the peptide includes, or is, the amino acid sequence of SEQ ID NO: 1962. In embodiments, the peptide includes, or is, the amino acid sequence of SEQ ID NO: 1963. In embodiments, the peptide includes, or is, the amino acid sequence of SEQ ID NO: 1964. In embodiments, the peptide includes, or is, the amino acid sequence of SEQ ID NO: 1965. In embodiments, the peptide includes, or is, the amino acid sequence of SEQ ID NO: 1966. In embodiments, the peptide includes, or is, the amino acid sequence of SEQ ID NO: 1967. In embodiments, the peptide includes, or is, the amino acid sequence of SEQ ID NO: 1968. In embodiments, the peptide includes, or is, the amino acid sequence of SEQ ID NO: 1969. In embodiments, the peptide includes, or is, the amino acid sequence of SEQ ID NO: 1970. In embodiments, the peptide includes, or is, the amino acid sequence of SEQ ID NO: 1971. In embodiments, the peptide includes, or is, the amino acid sequence of SEQ ID NO: 1972. In embodiments, the peptide includes, or is, the amino acid sequence of SEQ ID NO: 1973. In embodiments, the peptide includes, or is, the amino acid sequence of SEQ ID NO: 1974. In embodiments, the peptide includes, or is, the amino acid sequence of SEQ ID NO: 1975.

In embodiments, the peptide includes, or is, the amino acid sequence of SEQ ID NO: 1976. In embodiments, the peptide includes, or is, the amino acid sequence of SEQ ID NO: 1977. In embodiments, the peptide includes, or is, the amino acid sequence of SEQ ID NO: 1978. In embodiments, the peptide includes, or is, the amino acid sequence of SEQ ID NO: 1979. In embodiments, the peptide includes, or is, the amino acid sequence of SEQ ID NO: 1980. In embodiments, the peptide includes, or is, the amino acid sequence of SEQ ID NO: 1981. In embodiments, the peptide includes, or is, the amino acid sequence of SEQ ID NO: 1982. In embodiments, the peptide includes, or is, the amino acid sequence of SEQ ID NO: 1983. In embodiments, the peptide includes, or is, the amino acid sequence of SEQ ID NO: 1984. In embodiments, the peptide includes, or is, the amino acid sequence of SEQ ID NO: 1985. In embodiments, the peptide includes, or is, the amino acid sequence of SEQ ID NO: 1986. In embodiments, the peptide includes, or is, the amino acid sequence of SEQ ID NO: 1987. In embodiments, the peptide includes, or is, the amino acid sequence of SEQ ID NO: 1988. In embodiments, the peptide includes, or is, the amino acid sequence of SEQ ID NO: 1989. In embodiments, the peptide includes, or is, the amino acid sequence of SEQ ID NO: 1990. In embodiments, the peptide includes, or is, the amino acid sequence of SEQ ID NO: 1991. In embodiments, the peptide includes, or is, the amino acid sequence of SEQ ID NO: 1992. In embodiments, the peptide includes, or is, the amino acid sequence of SEQ ID NO: 1993. In embodiments, the peptide includes, or is, the amino acid sequence of SEQ ID NO: 1994. In embodiments, the peptide includes, or is, the amino acid sequence of SEQ ID NO: 1995. In embodiments, the peptide includes, or is, the amino acid sequence of SEQ ID NO: 1996. In embodiments, the peptide includes, or is, the amino acid sequence of SEQ ID NO: 1997. In embodiments, the peptide includes, or is, the amino acid sequence of SEQ ID NO: 1998. In embodiments, the peptide includes, or is, the amino acid sequence of SEQ ID NO: 1999. In embodiments, the peptide includes, or is, the amino acid sequence of SEQ ID NO: 2000.

In embodiments, the peptide includes, or is, the amino acid sequence of SEQ ID NO: 2001. In embodiments, the peptide includes, or is, the amino acid sequence of SEQ ID NO: 2002. In embodiments, the peptide includes, or is, the amino acid sequence of SEQ ID NO: 2003. In embodiments, the peptide includes, or is, the amino acid sequence of SEQ ID NO: 2004. In embodiments, the peptide includes, or is, the amino acid sequence of SEQ ID NO: 2005. In embodiments, the peptide includes, or is, the amino acid sequence of SEQ ID NO: 2006. In embodiments, the peptide includes, or is, the amino acid sequence of SEQ ID NO: 2007. In embodiments, the peptide includes, or is, the amino acid sequence of SEQ ID NO: 2008. In embodiments, the peptide includes, or is, the amino acid sequence of SEQ ID NO: 2009. In embodiments, the peptide includes, or is, the amino acid sequence of SEQ ID NO: 2010. In embodiments, the peptide includes, or is, the amino acid sequence of SEQ ID NO: 2011. In embodiments, the peptide includes, or is, the amino acid sequence of SEQ ID NO: 2012. In embodiments, the peptide includes, or is, the amino acid sequence of SEQ ID NO: 2013. In embodiments, the peptide includes, or is, the amino acid sequence of SEQ ID NO: 2014. In embodiments, the peptide includes, or is, the amino acid sequence of SEQ ID NO: 2015. In embodiments, the peptide includes, or is, the amino acid sequence of SEQ ID NO: 2016. In embodiments, the peptide includes, or is, the amino acid sequence of SEQ ID NO: 2017. In embodiments, the peptide includes, or is, the amino acid sequence of SEQ ID NO: 2018. In embodiments, the peptide includes, or is, the amino acid sequence of SEQ ID NO: 2019. In embodiments, the peptide includes, or is, the amino acid sequence of SEQ ID NO: 2020. In embodiments, the peptide includes, or is, the amino acid sequence of SEQ ID NO: 2021. In embodiments, the peptide includes, or is, the amino acid sequence of SEQ ID NO: 2022. In embodiments, the peptide includes, or is, the amino acid sequence of SEQ ID NO: 2023. In embodiments, the peptide includes, or is, the amino acid sequence of SEQ ID NO: 2024. In embodiments, the peptide includes, or is, the amino acid sequence of SEQ ID NO: 2025.

In embodiments, the peptide includes, or is, the amino acid sequence of SEQ ID NO: 2026. In embodiments, the peptide includes, or is, the amino acid sequence of SEQ ID NO: 2027. In embodiments, the peptide includes, or is, the amino acid sequence of SEQ ID NO: 2028. In embodiments, the peptide includes, or is, the amino acid sequence of SEQ ID NO: 2029. In embodiments, the peptide includes, or is, the amino acid sequence of SEQ ID NO: 2030. In embodiments, the peptide includes, or is, the amino acid sequence of SEQ ID NO: 2031. In embodiments, the peptide includes, or is, the amino acid sequence of SEQ ID NO: 2032. In embodiments, the peptide includes, or is, the amino acid sequence of SEQ ID NO: 2033. In embodiments, the peptide includes, or is, the amino acid sequence of SEQ ID NO: 2034. In embodiments, the peptide includes, or is, the amino acid sequence of SEQ ID NO: 2035. In embodiments, the peptide includes, or is, the amino acid sequence of SEQ ID NO: 2036. In embodiments, the peptide includes, or is, the amino acid sequence of SEQ ID NO: 2037. In embodiments, the peptide includes, or is, the amino acid sequence of SEQ ID NO: 2038. In embodiments, the peptide includes, or is, the amino acid sequence of SEQ ID NO: 2039. In embodiments, the peptide includes, or is, the amino acid sequence of SEQ ID NO: 2040. In embodiments, the peptide includes, or is, the amino acid sequence of SEQ ID NO: 2041. In embodiments, the peptide includes, or is, the amino acid sequence of SEQ ID NO: 2042. In embodiments, the peptide includes, or is, the amino acid sequence of SEQ ID NO: 2043. In embodiments, the peptide includes, or is, the amino acid sequence of SEQ ID NO: 2044. In embodiments, the peptide includes, or is, the amino acid sequence of SEQ ID NO: 2045. In embodiments, the peptide includes, or is, the amino acid sequence of SEQ ID NO: 2046. In embodiments, the peptide includes, or is, the amino acid sequence of SEQ ID NO: 2047. In embodiments, the peptide includes, or is, the amino acid sequence of SEQ ID NO: 2048. In embodiments, the peptide includes, or is, the amino acid sequence of SEQ ID NO: 2049. In embodiments, the peptide includes, or is, the amino acid sequence of SEQ ID NO: 2050.

In embodiments, the peptide includes, or is, the amino acid sequence of SEQ ID NO: 2051. In embodiments, the peptide includes, or is, the amino acid sequence of SEQ ID NO: 2052. In embodiments, the peptide includes, or is, the amino acid sequence of SEQ ID NO: 2053. In embodiments, the peptide includes, or is, the amino acid sequence of SEQ ID NO: 2054. In embodiments, the peptide includes, or is, the amino acid sequence of SEQ ID NO: 2055. In embodiments, the peptide includes, or is, the amino acid sequence of SEQ ID NO: 2056. In embodiments, the peptide includes, or is, the amino acid sequence of SEQ ID NO: 2057. In embodiments, the peptide includes, or is, the amino acid sequence of SEQ ID NO: 2058. In embodiments, the peptide includes, or is, the amino acid sequence of SEQ ID NO: 2059. In embodiments, the peptide includes, or is, the amino acid sequence of SEQ ID NO: 2060. In embodiments, the peptide includes, or is, the amino acid sequence of SEQ ID NO: 2061. In embodiments, the peptide includes, or is, the amino acid sequence of SEQ ID NO: 2062. In embodiments, the peptide includes, or is, the amino acid sequence of SEQ ID NO: 2063. In embodiments, the peptide includes, or is, the amino acid sequence of SEQ ID NO: 2064. In embodiments, the peptide includes, or is, the amino acid sequence of SEQ ID NO: 2065. In embodiments, the peptide includes, or is, the amino acid sequence of SEQ ID NO: 2066. In embodiments, the peptide includes, or is, the amino acid sequence of SEQ ID NO: 2067. In embodiments, the peptide includes, or is, the amino acid sequence of SEQ ID NO: 2068. In embodiments, the peptide includes, or is, the amino acid sequence of SEQ ID NO: 2069. In embodiments, the peptide includes, or is, the amino acid sequence of SEQ ID NO: 2070. In embodiments, the peptide includes, or is, the amino acid sequence of SEQ ID NO: 2071. In embodiments, the peptide includes, or is, the amino acid sequence of SEQ ID NO: 2072. In embodiments, the peptide includes, or is, the amino acid sequence of SEQ ID NO: 2073. In embodiments, the peptide includes, or is, the amino acid sequence of SEQ ID NO: 2074. In embodiments, the peptide includes, or is, the amino acid sequence of SEQ ID NO: 2075.

In embodiments, the peptide includes, or is, the amino acid sequence of SEQ ID NO: 2076. In embodiments, the peptide includes, or is, the amino acid sequence of SEQ ID NO: 2077. In embodiments, the peptide includes, or is, the amino acid sequence of SEQ ID NO: 2078. In embodiments, the peptide includes, or is, the amino acid sequence of SEQ ID NO: 2079. In embodiments, the peptide includes, or is, the amino acid sequence of SEQ ID NO: 2080. In embodiments, the peptide includes, or is, the amino acid sequence of SEQ ID NO: 2081. In embodiments, the peptide includes, or is, the amino acid sequence of SEQ ID NO: 2082. In embodiments, the peptide includes, or is, the amino acid sequence of SEQ ID NO: 2083. In embodiments, the peptide includes, or is, the amino acid sequence of SEQ ID NO: 2084. In embodiments, the peptide includes, or is, the amino acid sequence of SEQ ID NO: 2085. In embodiments, the peptide includes, or is, the amino acid sequence of SEQ ID NO: 2086. In embodiments, the peptide includes, or is, the amino acid sequence of SEQ ID NO: 2087. In embodiments, the peptide includes, or is, the amino acid sequence of SEQ ID NO: 2088. In embodiments, the peptide includes, or is, the amino acid sequence of SEQ ID NO: 2089. In embodiments, the peptide includes, or is, the amino acid sequence of SEQ ID NO: 2090. In embodiments, the peptide includes, or is, the amino acid sequence of SEQ ID NO: 2091. In embodiments, the peptide includes, or is, the amino acid sequence of SEQ ID NO: 2092. In embodiments, the peptide includes, or is, the amino acid sequence of SEQ ID NO: 2093. In embodiments, the peptide includes, or is, the amino acid sequence of SEQ ID NO: 2094. In embodiments, the peptide includes, or is, the amino acid sequence of SEQ ID NO: 2095. In embodiments, the peptide includes, or is, the amino acid sequence of SEQ ID NO: 2096. In embodiments, the peptide includes, or is, the amino acid sequence of SEQ ID NO: 2097. In embodiments, the peptide includes, or is, the amino acid sequence of SEQ ID NO: 2098. In embodiments, the peptide includes, or is, the amino acid sequence of SEQ ID NO: 2099. In embodiments, the peptide includes, or is, the amino acid sequence of SEQ ID NO: 2100.

In embodiments, the peptide includes, or is, the amino acid sequence of SEQ ID NO: 2101. In embodiments, the peptide includes, or is, the amino acid sequence of SEQ ID NO: 2102. In embodiments, the peptide includes, or is, the amino acid sequence of SEQ ID NO: 2103. In embodiments, the peptide includes, or is, the amino acid sequence of SEQ ID NO: 2104. In embodiments, the peptide includes, or is, the amino acid sequence of SEQ ID NO: 2105. In embodiments, the peptide includes, or is, the amino acid sequence of SEQ ID NO: 2106. In embodiments, the peptide includes, or is, the amino acid sequence of SEQ ID NO: 2107. In embodiments, the peptide includes, or is, the amino acid sequence of SEQ ID NO: 2108. In embodiments, the peptide includes, or is, the amino acid sequence of SEQ ID NO: 2109. In embodiments, the peptide includes, or is, the amino acid sequence of SEQ ID NO: 2110. In embodiments, the peptide includes, or is, the amino acid sequence of SEQ ID NO: 2111. In embodiments, the peptide includes, or is, the amino acid sequence of SEQ ID NO: 2112. In embodiments, the peptide includes, or is, the amino acid sequence of SEQ ID NO: 2113. In embodiments, the peptide includes, or is, the amino acid sequence of SEQ ID NO: 2114. In embodiments, the peptide includes, or is, the amino acid sequence of SEQ ID NO: 2115. In embodiments, the peptide includes, or is, the amino acid sequence of SEQ ID NO: 2116. In embodiments, the peptide includes, or is, the amino acid sequence of SEQ ID NO: 2117. In embodiments, the peptide includes, or is, the amino acid sequence of SEQ ID NO: 2118. In embodiments, the peptide includes, or is, the amino acid sequence of SEQ ID NO: 2119. In embodiments, the peptide includes, or is, the amino acid sequence of SEQ ID NO: 2120. In embodiments, the peptide includes, or is, the amino acid sequence of SEQ ID NO: 2121. In embodiments, the peptide includes, or is, the amino acid sequence of SEQ ID NO: 2122. In embodiments, the peptide includes, or is, the amino acid sequence of SEQ ID NO: 2123. In embodiments, the peptide includes, or is, the amino acid sequence of SEQ ID NO: 2124. In embodiments, the peptide includes, or is, the amino acid sequence of SEQ ID NO: 2125.

In embodiments, the peptide includes, or is, the amino acid sequence of SEQ ID NO: 2126. In embodiments, the peptide includes, or is, the amino acid sequence of SEQ ID NO: 2127. In embodiments, the peptide includes, or is, the amino acid sequence of SEQ ID NO: 2128. In embodiments, the peptide includes, or is, the amino acid sequence of SEQ ID NO: 2129. In embodiments, the peptide includes, or is, the amino acid sequence of SEQ ID NO: 2130. In embodiments, the peptide includes, or is, the amino acid sequence of SEQ ID NO: 2131. In embodiments, the peptide includes, or is, the amino acid sequence of SEQ ID NO: 2132. In embodiments, the peptide includes, or is, the amino acid sequence of SEQ ID NO: 2133. In embodiments, the peptide includes, or is, the amino acid sequence of SEQ ID NO: 2134. In embodiments, the peptide includes, or is, the amino acid sequence of SEQ ID NO: 2135. In embodiments, the peptide includes, or is, the amino acid sequence of SEQ ID NO: 2136. In embodiments, the peptide includes, or is, the amino acid sequence of SEQ ID NO: 2137. In embodiments, the peptide includes, or is, the amino acid sequence of SEQ ID NO: 2138. In embodiments, the peptide includes, or is, the amino acid sequence of SEQ ID NO: 2139. In embodiments, the peptide includes, or is, the amino acid sequence of SEQ ID NO: 2140. In embodiments, the peptide includes, or is, the amino acid sequence of SEQ ID NO: 2141. In embodiments, the peptide includes, or is, the amino acid sequence of SEQ ID NO: 2142. In embodiments, the peptide includes, or is, the amino acid sequence of SEQ ID NO: 2143. In embodiments, the peptide includes, or is, the amino acid sequence of SEQ ID NO: 2144. In embodiments, the peptide includes, or is, the amino acid sequence of SEQ ID NO: 2145. In embodiments, the peptide includes, or is, the amino acid sequence of SEQ ID NO: 2146. In embodiments, the peptide includes, or is, the amino acid sequence of SEQ ID NO: 2147. In embodiments, the peptide includes, or is, the amino acid sequence of SEQ ID NO: 2148. In embodiments, the peptide includes, or is, the amino acid sequence of SEQ ID NO: 2149. In embodiments, the peptide includes, or is, the amino acid sequence of SEQ ID NO: 2150.

In embodiments, the peptide includes, or is, the amino acid sequence of SEQ ID NO: 2151. In embodiments, the peptide includes, or is, the amino acid sequence of SEQ ID NO: 2152. In embodiments, the peptide includes, or is, the amino acid sequence of SEQ ID NO: 2153. In embodiments, the peptide includes, or is, the amino acid sequence of SEQ ID NO: 2154. In embodiments, the peptide includes, or is, the amino acid sequence of SEQ ID NO: 2155. In embodiments, the peptide includes, or is, the amino acid sequence of SEQ ID NO: 2156. In embodiments, the peptide includes, or is, the amino acid sequence of SEQ ID NO: 2157. In embodiments, the peptide includes, or is, the amino acid sequence of SEQ ID NO: 2158. In embodiments, the peptide includes, or is, the amino acid sequence of SEQ ID NO: 2159. In embodiments, the peptide includes, or is, the amino acid sequence of SEQ ID NO: 2160. In embodiments, the peptide includes, or is, the amino acid sequence of SEQ ID NO: 2161. In embodiments, the peptide includes, or is, the amino acid sequence of SEQ ID NO: 2162. In embodiments, the peptide includes, or is, the amino acid sequence of SEQ ID NO: 2163. In embodiments, the peptide includes, or is, the amino acid sequence of SEQ ID NO: 2164. In embodiments, the peptide includes, or is, the amino acid sequence of SEQ ID NO: 2165. In embodiments, the peptide includes, or is, the amino acid sequence of SEQ ID NO: 2166. In embodiments, the peptide includes, or is, the amino acid sequence of SEQ ID NO: 2167. In embodiments, the peptide includes, or is, the amino acid sequence of SEQ ID NO: 2168. In embodiments, the peptide includes, or is, the amino acid sequence of SEQ ID NO: 2169. In embodiments, the peptide includes, or is, the amino acid sequence of SEQ ID NO: 2170. In embodiments, the peptide includes, or is, the amino acid sequence of SEQ ID NO: 2171. In embodiments, the peptide includes, or is, the amino acid sequence of SEQ ID NO: 2172. In embodiments, the peptide includes, or is, the amino acid sequence of SEQ ID NO: 2173. In embodiments, the peptide includes, or is, the amino acid sequence of SEQ ID NO: 2174. In embodiments, the peptide includes, or is, the amino acid sequence of SEQ ID NO: 2175.

In embodiments, the peptide includes, or is, the amino acid sequence of SEQ ID NO: 2176. In embodiments, the peptide includes, or is, the amino acid sequence of SEQ ID NO: 2177. In embodiments, the peptide includes, or is, the amino acid sequence of SEQ ID NO: 2178. In embodiments, the peptide includes, or is, the amino acid sequence of SEQ ID NO: 2179. In embodiments, the peptide includes, or is, the amino acid sequence of SEQ ID NO: 2180. In embodiments, the peptide includes, or is, the amino acid sequence of SEQ ID NO: 2181. In embodiments, the peptide includes, or is, the amino acid sequence of SEQ ID NO: 2182. In embodiments, the peptide includes, or is, the amino acid sequence of SEQ ID NO: 2183. In embodiments, the peptide includes, or is, the amino acid sequence of SEQ ID NO: 2184. In embodiments, the peptide includes, or is, the amino acid sequence of SEQ ID NO: 2185. In embodiments, the peptide includes, or is, the amino acid sequence of SEQ ID NO: 2186. In embodiments, the peptide includes, or is, the amino acid sequence of SEQ ID NO: 2187. In embodiments, the peptide includes, or is, the amino acid sequence of SEQ ID NO: 2188. In embodiments, the peptide includes, or is, the amino acid sequence of SEQ ID NO: 2189. In embodiments, the peptide includes, or is, the amino acid sequence of SEQ ID NO: 2190. In embodiments, the peptide includes, or is, the amino acid sequence of SEQ ID NO: 2191. In embodiments, the peptide includes, or is, the amino acid sequence of SEQ ID NO: 2192. In embodiments, the peptide includes, or is, the amino acid sequence of SEQ ID NO: 2193. In embodiments, the peptide includes, or is, the amino acid sequence of SEQ ID NO: 2194. In embodiments, the peptide includes, or is, the amino acid sequence of SEQ ID NO: 2195. In embodiments, the peptide includes, or is, the amino acid sequence of SEQ ID NO: 2196. In embodiments, the peptide includes, or is, the amino acid sequence of SEQ ID NO: 2197. In embodiments, the peptide includes, or is, the amino acid sequence of SEQ ID NO: 2198. In embodiments, the peptide includes, or is, the amino acid sequence of SEQ ID NO: 2199. In embodiments, the peptide includes, or is, the amino acid sequence of SEQ ID NO: 2200.

In embodiments, the peptide includes, or is, the amino acid sequence of SEQ ID NO: 2201. In embodiments, the peptide includes, or is, the amino acid sequence of SEQ ID NO: 2202. In embodiments, the peptide includes, or is, the amino acid sequence of SEQ ID NO: 2203. In embodiments, the peptide includes, or is, the amino acid sequence of SEQ ID NO: 2204. In embodiments, the peptide includes, or is, the amino acid sequence of SEQ ID NO: 2205. In embodiments, the peptide includes, or is, the amino acid sequence of SEQ ID NO: 2206. In embodiments, the peptide includes, or is, the amino acid sequence of SEQ ID NO: 2207. In embodiments, the peptide includes, or is, the amino acid sequence of SEQ ID NO: 2208. In embodiments, the peptide includes, or is, the amino acid sequence of SEQ ID NO: 2209. In embodiments, the peptide includes, or is, the amino acid sequence of SEQ ID NO: 2210. In embodiments, the peptide includes, or is, the amino acid sequence of SEQ ID NO: 2211. In embodiments, the peptide includes, or is, the amino acid sequence of SEQ ID NO: 2212. In embodiments, the peptide includes, or is, the amino acid sequence of SEQ ID NO: 2213. In embodiments, the peptide includes, or is, the amino acid sequence of SEQ ID NO: 2214. In embodiments, the peptide includes, or is, the amino acid sequence of SEQ ID NO: 2215. In embodiments, the peptide includes, or is, the amino acid sequence of SEQ ID NO: 2216. In embodiments, the peptide includes, or is, the amino acid sequence of SEQ ID NO: 2217. In embodiments, the peptide includes, or is, the amino acid sequence of SEQ ID NO: 2218. In embodiments, the peptide includes, or is, the amino acid sequence of SEQ ID NO: 2219. In embodiments, the peptide includes, or is, the amino acid sequence of SEQ ID NO: 2220. In embodiments, the peptide includes, or is, the amino acid sequence of SEQ ID NO: 2221. In embodiments, the peptide includes, or is, the amino acid sequence of SEQ ID NO: 2222. In embodiments, the peptide includes, or is, the amino acid sequence of SEQ ID NO: 2223. In embodiments, the peptide includes, or is, the amino acid sequence of SEQ ID NO: 2224. In embodiments, the peptide includes, or is, the amino acid sequence of SEQ ID NO: 2225.

In embodiments, the peptide includes, or is, the amino acid sequence of SEQ ID NO: 2226. In embodiments, the peptide includes, or is, the amino acid sequence of SEQ ID NO: 2227. In embodiments, the peptide includes, or is, the amino acid sequence of SEQ ID NO: 2228. In embodiments, the peptide includes, or is, the amino acid sequence of SEQ ID NO: 2229. In embodiments, the peptide includes, or is, the amino acid sequence of SEQ ID NO: 2230. In embodiments, the peptide includes, or is, the amino acid sequence of SEQ ID NO: 2231. In embodiments, the peptide includes, or is, the amino acid sequence of SEQ ID NO: 2232. In embodiments, the peptide includes, or is, the amino acid sequence of SEQ ID NO: 2233. In embodiments, the peptide includes, or is, the amino acid sequence of SEQ ID NO: 2234. In embodiments, the peptide includes, or is, the amino acid sequence of SEQ ID NO: 2235. In embodiments, the peptide includes, or is, the amino acid sequence of SEQ ID NO: 2236. In embodiments, the peptide includes, or is, the amino acid sequence of SEQ ID NO: 2237. In embodiments, the peptide includes, or is, the amino acid sequence of SEQ ID NO: 2238. In embodiments, the peptide includes, or is, the amino acid sequence of SEQ ID NO: 2239. In embodiments, the peptide includes, or is, the amino acid sequence of SEQ ID NO: 2240. In embodiments, the peptide includes, or is, the amino acid sequence of SEQ ID NO: 2241. In embodiments, the peptide includes, or is, the amino acid sequence of SEQ ID NO: 2242. In embodiments, the peptide includes, or is, the amino acid sequence of SEQ ID NO: 2243. In embodiments, the peptide includes, or is, the amino acid sequence of SEQ ID NO: 2244. In embodiments, the peptide includes, or is, the amino acid sequence of SEQ ID NO: 2245. In embodiments, the peptide includes, or is, the amino acid sequence of SEQ ID NO: 2246. In embodiments, the peptide includes, or is, the amino acid sequence of SEQ ID NO: 2247. In embodiments, the peptide includes, or is, the amino acid sequence of SEQ ID NO: 2248. In embodiments, the peptide includes, or is, the amino acid sequence of SEQ ID NO: 2249. In embodiments, the peptide includes, or is, the amino acid sequence of SEQ ID NO: 2250.

In embodiments, the peptide includes, or is, the amino acid sequence of SEQ ID NO: 2251. In embodiments, the peptide includes, or is, the amino acid sequence of SEQ ID NO: 2252. In embodiments, the peptide includes, or is, the amino acid sequence of SEQ ID NO: 2253. In embodiments, the peptide includes, or is, the amino acid sequence of SEQ ID NO: 2254. In embodiments, the peptide includes, or is, the amino acid sequence of SEQ ID NO: 2255. In embodiments, the peptide includes, or is, the amino acid sequence of SEQ ID NO: 2256. In embodiments, the peptide includes, or is, the amino acid sequence of SEQ ID NO: 2257. In embodiments, the peptide includes, or is, the amino acid sequence of SEQ ID NO: 2258. In embodiments, the peptide includes, or is, the amino acid sequence of SEQ ID NO: 2259. In embodiments, the peptide includes, or is, the amino acid sequence of SEQ ID NO: 2260. In embodiments, the peptide includes, or is, the amino acid sequence of SEQ ID NO: 2261. In embodiments, the peptide includes, or is, the amino acid sequence of SEQ ID NO: 2262. In embodiments, the peptide includes, or is, the amino acid sequence of SEQ ID NO: 2263. In embodiments, the peptide includes, or is, the amino acid sequence of SEQ ID NO: 2264. In embodiments, the peptide includes, or is, the amino acid sequence of SEQ ID NO: 2265. In embodiments, the peptide includes, or is, the amino acid sequence of SEQ ID NO: 2266. In embodiments, the peptide includes, or is, the amino acid sequence of SEQ ID NO: 2267. In embodiments, the peptide includes, or is, the amino acid sequence of SEQ ID NO: 2268. In embodiments, the peptide includes, or is, the amino acid sequence of SEQ ID NO: 2269. In embodiments, the peptide includes, or is, the amino acid sequence of SEQ ID NO: 2270. In embodiments, the peptide includes, or is, the amino acid sequence of SEQ ID NO: 2271. In embodiments, the peptide includes, or is, the amino acid sequence of SEQ ID NO: 2272. In embodiments, the peptide includes, or is, the amino acid sequence of SEQ ID NO: 2273. In embodiments, the peptide includes, or is, the amino acid sequence of SEQ ID NO: 2274. In embodiments, the peptide includes, or is, the amino acid sequence of SEQ ID NO: 2275.

In embodiments, the peptide includes, or is, the amino acid sequence of SEQ ID NO: 2276. In embodiments, the peptide includes, or is, the amino acid sequence of SEQ ID NO: 2277. In embodiments, the peptide includes, or is, the amino acid sequence of SEQ ID NO: 2278. In embodiments, the peptide includes, or is, the amino acid sequence of SEQ ID NO: 2279. In embodiments, the peptide includes, or is, the amino acid sequence of SEQ ID NO: 2280. In embodiments, the peptide includes, or is, the amino acid sequence of SEQ ID NO: 2281. In embodiments, the peptide includes, or is, the amino acid sequence of SEQ ID NO: 2282. In embodiments, the peptide includes, or is, the amino acid sequence of SEQ ID NO: 2283. In embodiments, the peptide includes, or is, the amino acid sequence of SEQ ID NO: 2284. In embodiments, the peptide includes, or is, the amino acid sequence of SEQ ID NO: 2285. In embodiments, the peptide includes, or is, the amino acid sequence of SEQ ID NO: 2286. In embodiments, the peptide includes, or is, the amino acid sequence of SEQ ID NO: 2287. In embodiments, the peptide includes, or is, the amino acid sequence of SEQ ID NO: 2288. In embodiments, the peptide includes, or is, the amino acid sequence of SEQ ID NO: 2289. In embodiments, the peptide includes, or is, the amino acid sequence of SEQ ID NO: 2290. In embodiments, the peptide includes, or is, the amino acid sequence of SEQ ID NO: 2291. In embodiments, the peptide includes, or is, the amino acid sequence of SEQ ID NO: 2292. In embodiments, the peptide includes, or is, the amino acid sequence of SEQ ID NO: 2293. In embodiments, the peptide includes, or is, the amino acid sequence of SEQ ID NO: 2294. In embodiments, the peptide includes, or is, the amino acid sequence of SEQ ID NO: 2295. In embodiments, the peptide includes, or is, the amino acid sequence of SEQ ID NO: 2296. In embodiments, the peptide includes, or is, the amino acid sequence of SEQ ID NO: 2297. In embodiments, the peptide includes, or is, the amino acid sequence of SEQ ID NO: 2298. In embodiments, the peptide includes, or is, the amino acid sequence of SEQ ID NO: 2299. In embodiments, the peptide includes, or is, the amino acid sequence of SEQ ID NO: 2300.

In embodiments, the peptide includes, or is, the amino acid sequence of SEQ ID NO: 2301. In embodiments, the peptide includes, or is, the amino acid sequence of SEQ ID NO: 2302. In embodiments, the peptide includes, or is, the amino acid sequence of SEQ ID NO: 2303. In embodiments, the peptide includes, or is, the amino acid sequence of SEQ ID NO: 2304. In embodiments, the peptide includes, or is, the amino acid sequence of SEQ ID NO: 2305. In embodiments, the peptide includes, or is, the amino acid sequence of SEQ ID NO: 2306. In embodiments, the peptide includes, or is, the amino acid sequence of SEQ ID NO: 2307. In embodiments, the peptide includes, or is, the amino acid sequence of SEQ ID NO: 2308. In embodiments, the peptide includes, or is, the amino acid sequence of SEQ ID NO: 2309. In embodiments, the peptide includes, or is, the amino acid sequence of SEQ ID NO: 2310. In embodiments, the peptide includes, or is, the amino acid sequence of SEQ ID NO: 2311. In embodiments, the peptide includes, or is, the amino acid sequence of SEQ ID NO: 2312. In embodiments, the peptide includes, or is, the amino acid sequence of SEQ ID NO: 2313. In embodiments, the peptide includes, or is, the amino acid sequence of SEQ ID NO: 2314. In embodiments, the peptide includes, or is, the amino acid sequence of SEQ ID NO: 2315. In embodiments, the peptide includes, or is, the amino acid sequence of SEQ ID NO: 2316. In embodiments, the peptide includes, or is, the amino acid sequence of SEQ ID NO: 2317. In embodiments, the peptide includes, or is, the amino acid sequence of SEQ ID NO: 2318. In embodiments, the peptide includes, or is, the amino acid sequence of SEQ ID NO: 2319. In embodiments, the peptide includes, or is, the amino acid sequence of SEQ ID NO: 2320. In embodiments, the peptide includes, or is, the amino acid sequence of SEQ ID NO: 2321. In embodiments, the peptide includes, or is, the amino acid sequence of SEQ ID NO: 2322. In embodiments, the peptide includes, or is, the amino acid sequence of SEQ ID NO: 2323. In embodiments, the peptide includes, or is, the amino acid sequence of SEQ ID NO: 2324. In embodiments, the peptide includes, or is, the amino acid sequence of SEQ ID NO: 2325. In embodiments, the peptide includes, or is, the amino acid sequence of SEQ ID NO: 2326. In embodiments, the peptide includes, or is, the amino acid sequence of SEQ ID NO: 2327.

In embodiments, the peptide includes an amino acid sequence having 70% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 70% sequence identity to 10 contiguous amino acids of the sequence of one of SEQ ID NOs: 1-2327. In embodiments, the peptide includes an amino acid sequence having 70% sequence identity to 15 contiguous amino acids of the sequence of one of SEQ ID NOs: 1-2327. In embodiments, the peptide includes an amino acid sequence having 70% sequence identity to 20 contiguous amino acids of the sequence of one of SEQ ID NOs: 1-2327. In embodiments, the peptide includes an amino acid sequence having 70% sequence identity to 25 contiguous amino acids of the sequence of one of SEQ ID NOs: 1-2327. In embodiments, the peptide includes an amino acid sequence having 70% sequence identity to 30 contiguous amino acids of the sequence of one of SEQ ID NOs: 1-2327. In embodiments, the peptide includes an amino acid sequence having 70% sequence identity to 35 contiguous amino acids of the sequence of one of SEQ ID NOs: 1-2327. In embodiments, the peptide includes an amino acid sequence having 70% sequence identity to 36 contiguous amino acids of the sequence of one of SEQ ID NOs: 1-2327. In embodiments, the peptide includes an amino acid sequence having 70% sequence identity to 37 contiguous amino acids of the sequence of one of SEQ ID NOs: 1-2327. In embodiments, the peptide includes an amino acid sequence having 70% sequence identity to 38 contiguous amino acids of the sequence of one of SEQ ID NOs: 1-2327. In embodiments, the peptide includes an amino acid sequence having 70% sequence identity to 39 contiguous amino acids of the sequence of one of SEQ ID NOs: 1-2327.

In embodiments, the peptide includes an amino acid sequence having 75% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 75% sequence identity to 10 contiguous amino acids of the sequence of one of SEQ ID NOs: 1-2327. In embodiments, the peptide includes an amino acid sequence having 75% sequence identity to 15 contiguous amino acids of the sequence of one of SEQ ID NOs: 1-2327. In embodiments, the peptide includes an amino acid sequence having 75% sequence identity to 20 contiguous amino acids of the sequence of one of SEQ ID NOs: 1-2327. In embodiments, the peptide includes an amino acid sequence having 75% sequence identity to 25 contiguous amino acids of the sequence of one of SEQ ID NOs: 1-2327. In embodiments, the peptide includes an amino acid sequence having 75% sequence identity to 30 contiguous amino acids of the sequence of one of SEQ ID NOs: 1-2327. In embodiments, the peptide includes an amino acid sequence having 75% sequence identity to 35 contiguous amino acids of the sequence of one of SEQ ID NOs: 1-2327. In embodiments, the peptide includes an amino acid sequence having 75% sequence identity to 36 contiguous amino acids of the sequence of one of SEQ ID NOs: 1-2327. In embodiments, the peptide includes an amino acid sequence having 75% sequence identity to 37 contiguous amino acids of the sequence of one of SEQ ID NOs: 1-2327. In embodiments, the peptide includes an amino acid sequence having 75% sequence identity to 38 contiguous amino acids of the sequence of one of SEQ ID NOs: 1-2327. In embodiments, the peptide includes an amino acid sequence having 75% sequence identity to 39 contiguous amino acids of the sequence of one of SEQ ID NOs: 1-2327.

In embodiments, the peptide includes an amino acid sequence having 80% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 80% sequence identity to 10 contiguous amino acids of the sequence of one of SEQ ID NOs: 1-2327. In embodiments, the peptide includes an amino acid sequence having 80% sequence identity to 15 contiguous amino acids of the sequence of one of SEQ ID NOs: 1-2327. In embodiments, the peptide includes an amino acid sequence having 80% sequence identity to 20 contiguous amino acids of the sequence of one of SEQ ID NOs: 1-2327. In embodiments, the peptide includes an amino acid sequence having 80% sequence identity to 25 contiguous amino acids of the sequence of one of SEQ ID NOs: 1-2327. In embodiments, the peptide includes an amino acid sequence having 80% sequence identity to 30 contiguous amino acids of the sequence of one of SEQ ID NOs: 1-2327. In embodiments, the peptide includes an amino acid sequence having 80% sequence identity to 35 contiguous amino acids of the sequence of one of SEQ ID NOs: 1-2327. In embodiments, the peptide includes an amino acid sequence having 80% sequence identity to 36 contiguous amino acids of the sequence of one of SEQ ID NOs: 1-2327. In embodiments, the peptide includes an amino acid sequence having 80% sequence identity to 37 contiguous amino acids of the sequence of one of SEQ ID NOs: 1-2327. In embodiments, the peptide includes an amino acid sequence having 80% sequence identity to 38 contiguous amino acids of the sequence of one of SEQ ID NOs: 1-2327. In embodiments, the peptide includes an amino acid sequence having 80% sequence identity to 39 contiguous amino acids of the sequence of one of SEQ ID NOs: 1-2327.

In embodiments, the peptide includes an amino acid sequence having 85% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 85% sequence identity to 10 contiguous amino acids of the sequence of one of SEQ ID NOs: 1-2327. In embodiments, the peptide includes an amino acid sequence having 85% sequence identity to 15 contiguous amino acids of the sequence of one of SEQ ID NOs: 1-2327. In embodiments, the peptide includes an amino acid sequence having 85% sequence identity to 20 contiguous amino acids of the sequence of one of SEQ ID NOs: 1-2327. In embodiments, the peptide includes an amino acid sequence having 85% sequence identity to 25 contiguous amino acids of the sequence of one of SEQ ID NOs: 1-2327. In embodiments, the peptide includes an amino acid sequence having 85% sequence identity to 30 contiguous amino acids of the sequence of one of SEQ ID NOs: 1-2327. In embodiments, the peptide includes an amino acid sequence having 85% sequence identity to 35 contiguous amino acids of the sequence of one of SEQ ID NOs: 1-2327. In embodiments, the peptide includes an amino acid sequence having 85% sequence identity to 36 contiguous amino acids of the sequence of one of SEQ ID NOs: 1-2327. In embodiments, the peptide includes an amino acid sequence having 85% sequence identity to 37 contiguous amino acids of the sequence of one of SEQ ID NOs: 1-2327. In embodiments, the peptide includes an amino acid sequence having 85% sequence identity to 38 contiguous amino acids of the sequence of one of SEQ ID NOs: 1-2327. In embodiments, the peptide includes an amino acid sequence having 85% sequence identity to 39 contiguous amino acids of the sequence of one of SEQ ID NOs: 1-2327.

In embodiments, the peptide includes an amino acid sequence having 90% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 90% sequence identity to 10 contiguous amino acids of the sequence of one of SEQ ID NOs: 1-2327. In embodiments, the peptide includes an amino acid sequence having 90% sequence identity to 15 contiguous amino acids of the sequence of one of SEQ ID NOs: 1-2327. In embodiments, the peptide includes an amino acid sequence having 90% sequence identity to 20 contiguous amino acids of the sequence of one of SEQ ID NOs: 1-2327. In embodiments, the peptide includes an amino acid sequence having 90% sequence identity to 25 contiguous amino acids of the sequence of one of SEQ ID NOs: 1-2327. In embodiments, the peptide includes an amino acid sequence having 90% sequence identity to 30 contiguous amino acids of the sequence of one of SEQ ID NOs: 1-2327. In embodiments, the peptide includes an amino acid sequence having 90% sequence identity to 35 contiguous amino acids of the sequence of one of SEQ ID NOs: 1-2327. In embodiments, the peptide includes an amino acid sequence having 90% sequence identity to 36 contiguous amino acids of the sequence of one of SEQ ID NOs: 1-2327. In embodiments, the peptide includes an amino acid sequence having 90% sequence identity to 37 contiguous amino acids of the sequence of one of SEQ ID NOs: 1-2327. In embodiments, the peptide includes an amino acid sequence having 90% sequence identity to 38 contiguous amino acids of the sequence of one of SEQ ID NOs: 1-2327. In embodiments, the peptide includes an amino acid sequence having 90% sequence identity to 39 contiguous amino acids of the sequence of one of SEQ ID NOs: 1-2327.

In embodiments, the peptide includes an amino acid sequence having 95% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 95% sequence identity to 10 contiguous amino acids of the sequence of one of SEQ ID NOs: 1-2327. In embodiments, the peptide includes an amino acid sequence having 95% sequence identity to 15 contiguous amino acids of the sequence of one of SEQ ID NOs: 1-2327. In embodiments, the peptide includes an amino acid sequence having 95% sequence identity to 20 contiguous amino acids of the sequence of one of SEQ ID NOs: 1-2327. In embodiments, the peptide includes an amino acid sequence having 95% sequence identity to 25 contiguous amino acids of the sequence of one of SEQ ID NOs: 1-2327. In embodiments, the peptide includes an amino acid sequence having 95% sequence identity to 30 contiguous amino acids of the sequence of one of SEQ ID NOs: 1-2327. In embodiments, the peptide includes an amino acid sequence having 95% sequence identity to 35 contiguous amino acids of the sequence of one of SEQ ID NOs: 1-2327. In embodiments, the peptide includes an amino acid sequence having 95% sequence identity to 36 contiguous amino acids of the sequence of one of SEQ ID NOs: 1-2327. In embodiments, the peptide includes an amino acid sequence having 95% sequence identity to 37 contiguous amino acids of the sequence of one of SEQ ID NOs: 1-2327. In embodiments, the peptide includes an amino acid sequence having 95% sequence identity to 38 contiguous amino acids of the sequence of one of SEQ ID NOs: 1-2327. In embodiments, the peptide includes an amino acid sequence having 95% sequence identity to 39 contiguous amino acids of the sequence of one of SEQ ID NOs: 1-2327.

In embodiments, the peptide includes an amino acid sequence having 96% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 96% sequence identity to 10 contiguous amino acids of the sequence of one of SEQ ID NOs: 1-2327. In embodiments, the peptide includes an amino acid sequence having 96% sequence identity to 15 contiguous amino acids of the sequence of one of SEQ ID NOs: 1-2327. In embodiments, the peptide includes an amino acid sequence having 96% sequence identity to 20 contiguous amino acids of the sequence of one of SEQ ID NOs: 1-2327. In embodiments, the peptide includes an amino acid sequence having 96% sequence identity to 25 contiguous amino acids of the sequence of one of SEQ ID NOs: 1-2327. In embodiments, the peptide includes an amino acid sequence having 96% sequence identity to 30 contiguous amino acids of the sequence of one of SEQ ID NOs: 1-2327. In embodiments, the peptide includes an amino acid sequence having 96% sequence identity to 35 contiguous amino acids of the sequence of one of SEQ ID NOs: 1-2327. In embodiments, the peptide includes an amino acid sequence having 96% sequence identity to 36 contiguous amino acids of the sequence of one of SEQ ID NOs: 1-2327. In embodiments, the peptide includes an amino acid sequence having 96% sequence identity to 37 contiguous amino acids of the sequence of one of SEQ ID NOs: 1-2327. In embodiments, the peptide includes an amino acid sequence having 96% sequence identity to 38 contiguous amino acids of the sequence of one of SEQ ID NOs: 1-2327. In embodiments, the peptide includes an amino acid sequence having 96% sequence identity to 39 contiguous amino acids of the sequence of one of SEQ ID NOs: 1-2327.

In embodiments, the peptide includes an amino acid sequence having 97% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 97% sequence identity to 10 contiguous amino acids of the sequence of one of SEQ ID NOs: 1-2327. In embodiments, the peptide includes an amino acid sequence having 97% sequence identity to 15 contiguous amino acids of the sequence of one of SEQ ID NOs: 1-2327. In embodiments, the peptide includes an amino acid sequence having 97% sequence identity to 20 contiguous amino acids of the sequence of one of SEQ ID NOs: 1-2327. In embodiments, the peptide includes an amino acid sequence having 97% sequence identity to 25 contiguous amino acids of the sequence of one of SEQ ID NOs: 1-2327. In embodiments, the peptide includes an amino acid sequence having 97% sequence identity to 30 contiguous amino acids of the sequence of one of SEQ ID NOs: 1-2327. In embodiments, the peptide includes an amino acid sequence having 97% sequence identity to 35 contiguous amino acids of the sequence of one of SEQ ID NOs: 1-2327. In embodiments, the peptide includes an amino acid sequence having 97% sequence identity to 36 contiguous amino acids of the sequence of one of SEQ ID NOs: 1-2327. In embodiments, the peptide includes an amino acid sequence having 97% sequence identity to 37 contiguous amino acids of the sequence of one of SEQ ID NOs: 1-2327. In embodiments, the peptide includes an amino acid sequence having 97% sequence identity to 38 contiguous amino acids of the sequence of one of SEQ ID NOs: 1-2327. In embodiments, the peptide includes an amino acid sequence having 97% sequence identity to 39 contiguous amino acids of the sequence of one of SEQ ID NOs: 1-2327.

In embodiments, the peptide includes an amino acid sequence having 98% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 98% sequence identity to 10 contiguous amino acids of the sequence of one of SEQ ID NOs: 1-2327. In embodiments, the peptide includes an amino acid sequence having 98% sequence identity to 15 contiguous amino acids of the sequence of one of SEQ ID NOs: 1-2327. In embodiments, the peptide includes an amino acid sequence having 98% sequence identity to 20 contiguous amino acids of the sequence of one of SEQ ID NOs: 1-2327. In embodiments, the peptide includes an amino acid sequence having 98% sequence identity to 25 contiguous amino acids of the sequence of one of SEQ ID NOs: 1-2327. In embodiments, the peptide includes an amino acid sequence having 98% sequence identity to 30 contiguous amino acids of the sequence of one of SEQ ID NOs: 1-2327. In embodiments, the peptide includes an amino acid sequence having 98% sequence identity to 35 contiguous amino acids of the sequence of one of SEQ ID NOs: 1-2327. In embodiments, the peptide includes an amino acid sequence having 98% sequence identity to 36 contiguous amino acids of the sequence of one of SEQ ID NOs: 1-2327. In embodiments, the peptide includes an amino acid sequence having 98% sequence identity to 37 contiguous amino acids of the sequence of one of SEQ ID NOs: 1-2327. In embodiments, the peptide includes an amino acid sequence having 98% sequence identity to 38 contiguous amino acids of the sequence of one of SEQ ID NOs: 1-2327. In embodiments, the peptide includes an amino acid sequence having 98% sequence identity to 39 contiguous amino acids of the sequence of one of SEQ ID NOs: 1-2327.

In embodiments, the peptide includes an amino acid sequence having 99% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 99% sequence identity to 10 contiguous amino acids of the sequence of one of SEQ ID NOs: 1-2327. In embodiments, the peptide includes an amino acid sequence having 99% sequence identity to 15 contiguous amino acids of the sequence of one of SEQ ID NOs: 1-2327. In embodiments, the peptide includes an amino acid sequence having 99% sequence identity to 20 contiguous amino acids of the sequence of one of SEQ ID NOs: 1-2327. In embodiments, the peptide includes an amino acid sequence having 99% sequence identity to 25 contiguous amino acids of the sequence of one of SEQ ID NOs: 1-2327. In embodiments, the peptide includes an amino acid sequence having 99% sequence identity to 30 contiguous amino acids of the sequence of one of SEQ ID NOs: 1-2327. In embodiments, the peptide includes an amino acid sequence having 99% sequence identity to 35 contiguous amino acids of the sequence of one of SEQ ID NOs: 1-2327. In embodiments, the peptide includes an amino acid sequence having 99% sequence identity to 36 contiguous amino acids of the sequence of one of SEQ ID NOs: 1-2327. In embodiments, the peptide includes an amino acid sequence having 99% sequence identity to 37 contiguous amino acids of the sequence of one of SEQ ID NOs: 1-2327. In embodiments, the peptide includes an amino acid sequence having 99% sequence identity to 38 contiguous amino acids of the sequence of one of SEQ ID NOs: 1-2327. In embodiments, the peptide includes an amino acid sequence having 99% sequence identity to 39 contiguous amino acids of the sequence of one of SEQ ID NOs: 1-2327.

In embodiments, the peptide includes an amino acid sequence having 100% sequence identity to 10, 15, 20, 25, 30, 35, 36, 37, 38, or 39 contiguous amino acids of the sequence of one of SEQ ID NOs: 1-2327. In embodiments, the peptide includes an amino acid sequence having 100% sequence identity to 10 contiguous amino acids of the sequence of one of SEQ ID NOs: 1-2327. In embodiments, the peptide includes an amino acid sequence having 100% sequence identity to 15 contiguous amino acids of the sequence of one of SEQ ID NOs: 1-2327. In embodiments, the peptide includes an amino acid sequence having 100% sequence identity to 20 contiguous amino acids of the sequence of one of SEQ ID NOs: 1-2327. In embodiments, the peptide includes an amino acid sequence having 100% sequence identity to 25 contiguous amino acids of the sequence of one of SEQ ID NOs: 1-2327. In embodiments, the peptide includes an amino acid sequence having 100% sequence identity to 30 contiguous amino acids of the sequence of one of SEQ ID NOs: 1-2327. In embodiments, the peptide includes an amino acid sequence having 100% sequence identity to 35 contiguous amino acids of the sequence of one of SEQ ID NOs: 1-2327. In embodiments, the peptide includes an amino acid sequence having 100% sequence identity to 36 contiguous amino acids of the sequence of one of SEQ ID NOs: 1-2327. In embodiments, the peptide includes an amino acid sequence having 100% sequence identity to 37 contiguous amino acids of the sequence of one of SEQ ID NOs: 1-2327. In embodiments, the peptide includes an amino acid sequence having 100% sequence identity to 38 contiguous amino acids of the sequence of one of SEQ ID NOs: 1-2327. In embodiments, the peptide includes an amino acid sequence having 100% sequence identity to 39 contiguous amino acids of the sequence of one of SEQ ID NOs: 1-2327.

In embodiments, the peptide is an amino acid sequence having 100% sequence identity to 10, 15, 20, 25, 30, 35, 36, 37, 38, or 39 contiguous amino acids of the sequence of one of SEQ ID NOs: 1-2327. In embodiments, the peptide is an amino acid sequence having 100% sequence identity to 10 contiguous amino acids of the sequence of one of SEQ ID NOs: 1-2327. In embodiments, the peptide is an amino acid sequence having 100% sequence identity to 15 contiguous amino acids of the sequence of one of SEQ ID NOs: 1-2327. In embodiments, the peptide is an amino acid sequence having 100% sequence identity to 20 contiguous amino acids of the sequence of one of SEQ ID NOs: 1-2327. In embodiments, the peptide is an amino acid sequence having 100% sequence identity to 25 contiguous amino acids of the sequence of one of SEQ ID NOs: 1-2327. In embodiments, the peptide is an amino acid sequence having 100% sequence identity to 30 contiguous amino acids of the sequence of one of SEQ ID NOs: 1-2327. In embodiments, the peptide is an amino acid sequence having 100% sequence identity to 35 contiguous amino acids of the sequence of one of SEQ ID NOs: 1-2327. In embodiments, the peptide is an amino acid sequence having 100% sequence identity to 36 contiguous amino acids of the sequence of one of SEQ ID NOs: 1-2327. In embodiments, the peptide is an amino acid sequence having 100% sequence identity to 37 contiguous amino acids of the sequence of one of SEQ ID NOs: 1-2327. In embodiments, the peptide is an amino acid sequence having 100% sequence identity to 38 contiguous amino acids of the sequence of one of SEQ ID NOs: 1-2327. In embodiments, the peptide is an amino acid sequence having 100% sequence identity to 39 contiguous amino acids of the sequence of one of SEQ ID NOs: 1-2327.

In embodiments, the peptide includes an amino acid including a side chain that is at least partially protonated at a pH of about 7.3. In embodiments, the peptide includes an amino acid including a side chain that is at least partially protonated at a pH of 7.3.

In embodiments, the peptide includes an amino acid including an alcohol or thiol containing side chain. In embodiments, the peptide includes an amino acid including an alcohol containing side chain. In embodiments, the peptide includes an amino acid including a thiol containing side chain.

In embodiments, the peptide includes an amino acid including a side chain that is at least partially deprotonated at a pH of about 7.3. In embodiments, the peptide includes an amino acid including a side chain that is at least partially deprotonated at a pH of 7.3.

In embodiments, the stabilization moiety includes a cyclization moiety, a PEGylation moiety, a glycosylation moiety, a lipidation moiety, a hydroxylation moiety, a methylation moiety, an acetylation moiety, a sulfonation moiety, an amidation moiety, an esterification moiety, an N-terminus peptide cap moiety, or a C-terminus peptide cap moiety. In embodiments, the stabilization moiety includes a cyclization moiety. In embodiments, the stabilization moiety includes a PEGylation moiety. In embodiments, the stabilization moiety includes a glycosylation moiety. In embodiments, the stabilization moiety includes a lipidation moiety. In embodiments, the stabilization moiety includes a hydroxylation moiety, a methylation moiety. In embodiments, the stabilization moiety includes an acetylation moiety. In embodiments, the stabilization moiety includes a sulfonation moiety. In embodiments, the stabilization moiety includes an amidation moiety. In embodiments, the stabilization moiety includes an esterification moiety. In embodiments, the stabilization moiety includes an N-terminus peptide cap moiety. In embodiments, the stabilization moiety includes a C-terminus peptide cap moiety.

In embodiments, the stabilization moiety is a cyclization moiety, a PEGylation moiety, a glycosylation moiety, a lipidation moiety, a hydroxylation moiety, a methylation moiety, an acetylation moiety, a sulfonation moiety, an amidation moiety, an esterification moiety, an N-terminus peptide cap moiety, or a C-terminus peptide cap moiety. In embodiments, the stabilization moiety is a cyclization moiety. In embodiments, the stabilization moiety is a PEGylation moiety. In embodiments, the stabilization moiety is a glycosylation moiety. In embodiments, the stabilization moiety is a lipidation moiety. In embodiments, the stabilization moiety is a hydroxylation moiety, a methylation moiety. In embodiments, the stabilization moiety is an acetylation moiety. In embodiments, the stabilization moiety is a sulfonation moiety. In embodiments, the stabilization moiety is an amidation moiety. In embodiments, the stabilization moiety is an esterification moiety. In embodiments, the stabilization moiety is an N-terminus peptide cap moiety. In embodiments, the stabilization moiety is a C-terminus peptide cap moiety.

In embodiments, the cyclization moiety includes a head-to-tail cyclization moiety or a side-chain cyclization moiety. In embodiments, the cyclization moiety includes a head-to-tail cyclization moiety. In embodiments, the cyclization moiety includes a side-chain cyclization moiety. In embodiments, the cyclization moiety is a head-to-tail cyclization moiety or a side-chain cyclization moiety. In embodiments, the cyclization moiety is a head-to-tail cyclization moiety. In embodiments, the cyclization moiety is a side-chain cyclization moiety.

In embodiments, the cyclization moiety includes hydrocarbon stapling. In embodiments, the cyclization moiety is hydrocarbon stapling.

In embodiments, the peptide is from about 40 amino acids to about 120 amino acids in length. In embodiments, the peptide is from about 50 amino acids to about 120 amino acids in length. In embodiments, the peptide is from about 60 amino acids to about 120 amino acids in length. In embodiments, the peptide is from about 70 amino acids to about 120 amino acids in length. In embodiments, the peptide is from about 80 amino acids to about 120 amino acids in length. In embodiments, the peptide is from about 90 amino acids to about 120 amino acids in length. In embodiments, the peptide is from about 100 amino acids to about 120 amino acids in length. In embodiments, the peptide is from about 110 amino acids to about 120 amino acids in length.

In embodiments, the peptide is from 40 amino acids to 110 amino acids in length. In embodiments, the peptide is from 40 amino acids to 100 amino acids in length. In embodiments, the peptide is from 40 amino acids to 90 amino acids in length. In embodiments, the peptide is from 40 amino acids to 80 amino acids in length. In embodiments, the peptide is from 40 amino acids to 70 amino acids in length. In embodiments, the peptide is from 40 amino acids to 60 amino acids in length. In embodiments, the peptide is from 40 amino acids to about 50 acids in length.

In embodiments, the peptide is from 40 amino acids to 120 amino acids in length. In embodiments, the peptide is from 50 amino acids to 120 amino acids in length. In embodiments, the peptide is from 60 amino acids to 120 amino acids in length. In embodiments, the peptide is from 70 amino acids to 120 amino acids in length. In embodiments, the peptide is from 80 amino acids to 120 amino acids in length. In embodiments, the peptide is from 90 amino acids to 120 amino acids in length. In embodiments, the peptide is from 100 amino acids to 120 amino acids in length. In embodiments, the peptide is from 110 amino acids to 120 amino acids in length.

In embodiments, the peptide is from 40 amino acids to 110 amino acids in length. In embodiments, the peptide is from 40 amino acids to 100 amino acids in length. In embodiments, the peptide is from 40 amino acids to 90 amino acids in length. In embodiments, the peptide is from 40 amino acids to 80 amino acids in length. In embodiments, the peptide is from 40 amino acids to 70 amino acids in length. In embodiments, the peptide is from 40 amino acids to 60 amino acids in length. In embodiments, the peptide is from 40 amino acids to 50 amino acids in length.

In embodiments, the peptide further includes a cell-penetrating peptide. In embodiments, the cell-penetrating peptide is a TAT peptide, a penetratin peptide, a buforin II peptide, a transportan peptide, a model amphipathic peptide (MAP), a K-FGF peptide, a Ku70 peptide, a prion peptide, a pVEC peptide, a Pep-1 peptide, a SynB1 peptide, a Pep-7 peptide, an HN-1 peptide, a maurocalcine peptide, a CADY peptide, or a p28 peptide. In embodiments, the cell-penetrating peptide is a TAT peptide. In embodiments, the cell-penetrating peptide is a penetratin peptide. In embodiments, the cell-penetrating peptide is a buforin II peptide. In embodiments, the cell-penetrating peptide is a transportan peptide. In embodiments, the cell-penetrating peptide is a model amphipathic peptide (MAP). In embodiments, the cell-penetrating peptide is a K-FGF peptide. In embodiments, the cell-penetrating peptide is a Ku70 peptide. In embodiments, the cell-penetrating peptide is a prion peptide. In embodiments, the cell-penetrating peptide is a pVEC peptide. In embodiments, the cell-penetrating peptide is a Pep-1 peptide. In embodiments, the cell-penetrating peptide is a SynB1 peptide. In embodiments, the cell-penetrating peptide is a Pep-7 peptide. In embodiments, the cell-penetrating peptide is an HN-1 peptide. In embodiments, the cell-penetrating peptide is a maurocalcine peptide. In embodiments, the cell-penetrating peptide is a CADY peptide. In embodiments, the cell-penetrating peptide is a p28 peptide.

In embodiments, the cell-penetrating peptide includes the amino acid sequence of any one of SEQ ID NOs: 2328-2348. In embodiments, the cell-penetrating peptide includes the amino acid sequence of SEQ ID NO:2328. In embodiments, the cell-penetrating peptide includes the amino acid sequence of SEQ ID NO:2329. In embodiments, the cell-penetrating peptide includes the amino acid sequence of SEQ ID NO:2330. In embodiments, the cell-penetrating peptide includes the amino acid sequence of SEQ ID NO:2331. In embodiments, the cell-penetrating peptide includes the amino acid sequence of SEQ ID NO:2332. In embodiments, the cell-penetrating peptide includes the amino acid sequence of SEQ ID NO: 2333. In embodiments, the cell-penetrating peptide includes the amino acid sequence of SEQ ID NO:2334. In embodiments, the cell-penetrating peptide includes the amino acid sequence of SEQ ID NO:2335. In embodiments, the cell-penetrating peptide includes the amino acid sequence of SEQ ID NO:2336. In embodiments, the cell-penetrating peptide includes the amino acid sequence of SEQ ID NO:2337. In embodiments, the cell-penetrating peptide includes the amino acid sequence of SEQ ID NO:2338. In embodiments, the cell-penetrating peptide includes the amino acid sequence of SEQ ID NO:2339. In embodiments, the cell-penetrating peptide includes the amino acid sequence of SEQ ID NO:2340. In embodiments, the cell-penetrating peptide includes the amino acid sequence of SEQ ID NO: 2341. In embodiments, the cell-penetrating peptide includes the amino acid sequence of SEQ ID NO:2342. In embodiments, the cell-penetrating peptide includes the amino acid sequence of SEQ ID NO:2343. In embodiments, the cell-penetrating peptide includes the amino acid sequence of SEQ ID NO:2344. In embodiments, the cell-penetrating peptide includes the amino acid sequence of SEQ ID NO:2345. In embodiments, the cell-penetrating peptide includes the amino acid sequence of SEQ ID NO:2346. In embodiments, the cell-penetrating peptide includes the amino acid sequence of SEQ ID NO:2347. In embodiments, the cell-penetrating peptide includes the amino acid sequence of SEQ ID NO:2348.

In embodiments, the cell-penetrating peptide is attached to the peptide via a linker. In embodiments, the cell-penetrating peptide is attached to the peptide through a linker. In embodiments, the cell-penetrated peptide is attached to the N-terminus or the C-terminus of the peptide through a linker. In embodiments, the cell-penetrated peptide is attached to the N-terminus of the peptide through a linker. In embodiments, the cell-penetrated peptide is attached to the N-terminus of the peptide through a linker. In embodiments, the linker is an amino acid linker, a peptide linker, a chemical linker, or a bond. In embodiments, the linker is an amino acid linker. In embodiments, the linker is a peptide linker. In embodiments, the linker is a chemical linker. In embodiments, the linker is a peptide linker. In embodiments, the linker is a bond. In embodiments, the bond is an amide bond. In embodiments, the bond is a non-covalent bond. In embodiments, the bond is a covalent bond. Linkers provided herein can be a bond (e.g., covalent bond) and are referred to herein as a direct attachment. In embodiments, the cell-penetrating peptide is attached to the peptide through a direct covalent attachment.

In embodiments, the cell-penetrating peptide is the amino acid sequence of any one of SEQ ID NOs: 2328-2348. In embodiments, the cell-penetrating peptide is the amino acid sequence of SEQ ID NO:2328. In embodiments, the cell-penetrating peptide is the amino acid sequence of SEQ ID NO:2329. In embodiments, the cell-penetrating peptide is the amino acid sequence of SEQ ID NO:2330. In embodiments, the cell-penetrating peptide is the amino acid sequence of SEQ ID NO:2331. In embodiments, the cell-penetrating peptide is the amino acid sequence of SEQ ID NO:2332. In embodiments, the cell-penetrating peptide is the amino acid sequence of SEQ ID NO:2333. In embodiments, the cell-penetrating peptide is the amino acid sequence of SEQ ID NO:2334. In embodiments, the cell-penetrating peptide is the amino acid sequence of SEQ ID NO:2335. In embodiments, the cell-penetrating peptide is the amino acid sequence of SEQ ID NO:2336. In embodiments, the cell-penetrating peptide is the amino acid sequence of SEQ ID NO:2337. In embodiments, the cell-penetrating peptide is the amino acid sequence of SEQ ID NO: 2338. In embodiments, the cell-penetrating peptide is the amino acid sequence of SEQ ID NO: 2339. In embodiments, the cell-penetrating peptide is the amino acid sequence of SEQ ID NO:2340. In embodiments, the cell-penetrating peptide is the amino acid sequence of SEQ ID NO:2341. In embodiments, the cell-penetrating peptide is the amino acid sequence of SEQ ID NO:2342. In embodiments, the cell-penetrating peptide is the amino acid sequence of SEQ ID NO:2343. In embodiments, the cell-penetrating peptide is the amino acid sequence of SEQ ID NO:2344. In embodiments, the cell-penetrating peptide is the amino acid sequence of SEQ ID NO:2345. In embodiments, the cell-penetrating peptide is the amino acid sequence of SEQ ID NO:2346. In embodiments, the cell-penetrating peptide is the amino acid sequence of SEQ ID NO:2347. In embodiments, the cell-penetrating peptide is the amino acid sequence of SEQ ID NO:2348. In embodiments, the cell-penetrating peptide is attached to the peptide via a linker.

In embodiments, the peptide includes a cell-penetrating peptide. In embodiments, the cell-penetrating peptide includes the amino acid sequence of any one of SEQ ID NOs: 2328-2348. In embodiments, the cell-penetrating peptide is the amino acid sequence of any one of SEQ ID NOs: 2328-2348. In embodiments, the cell-penetrating peptide is attached to the peptide via a linker. In embodiments, the cell-penetrating peptide is attached to the peptide through a linker. In embodiments, the cell-penetrated peptide is attached to the N-terminus or the C-terminus of the peptide through a linker. In embodiments, the cell-penetrated peptide is attached to the N-terminus of the peptide through a linker. In embodiments, the cell-penetrated peptide is attached to the N-terminus of the peptide through a linker.

In embodiments, the linker is a peptide linker. In embodiments, the linker is from about 1 to about 50 amino acids in length. In embodiments, the linker is from about 2 to about 50 amino acids in length. In embodiments, the linker is from about 3 to about 50 amino acids in length. In embodiments, the linker is from about 4 to about 50 amino acids in length. In embodiments, the linker is from about 5 to about 50 amino acids in length. In embodiments, the linker is from about 6 to about 50 amino acids in length. In embodiments, the linker is from about 7 to about 50 amino acids in length. In embodiments, the linker is from about 8 to about 50 amino acids in length. In embodiments, the linker is from about 9 to about 50 amino acids in length. In embodiments, the linker is from about 10 to about 50 amino acids in length. In embodiments, the linker is from about 11 to about 50 amino acids in length. In embodiments, the linker is from about 12 to about 50 amino acids in length. In embodiments, the linker is from about 13 to about 50 amino acids in length. In embodiments, the linker is from about 14 to about 50 amino acids in length. In embodiments, the linker is from about 15 to about 50 amino acids in length. In embodiments, the linker is from about 20 to about 50 amino acids in length. In embodiments, the linker is from about 25 to about 50 amino acids in length. In embodiments, the linker is from about 30 to about 50 amino acids in length. In embodiments, the linker is from about 35 to about 50 amino acids in length. In embodiments, the linker is from about 40 to about 50 amino acids in length. In embodiments, the linker is from about 45 to about 50 amino acids in length.

In embodiments, the linker is from about 1 to about 45 amino acids in length. In embodiments, the linker is from about 1 to about 40 amino acids in length. In embodiments, the linker is from about 1 to about 35 amino acids in length. In embodiments, the linker is from about 1 to about 30 amino acids in length. In embodiments, the linker is from about 1 to about 25 amino acids in length. In embodiments, the linker is from about 1 to about 20 amino acids in length. In embodiments, the linker is from about 1 to about 15 amino acids in length. In embodiments, the linker is from about 1 to about 14 amino acids in length. In embodiments, the linker is from about 1 to about 13 amino acids in length. In embodiments, the linker is from about 1 to about 12 amino acids in length. In embodiments, the linker is from about 1 to about 11 amino acids in length. In embodiments, the linker is from about 1 to about 10 amino acids in length. In embodiments, the linker is from about 1 to about 9 amino acids in length. In embodiments, the linker is from about 1 to about 8 amino acids in length. In embodiments, the linker is from about 1 to about 7 amino acids in length. In embodiments, the linker is from about 1 to about 6 amino acids in length. In embodiments, the linker is from about 1 to about 5 amino acids in length. In embodiments, the linker is from about 1 to about 4 amino acids in length. In embodiments, the linker is from about 1 to about 3 amino acids in length. In embodiments, the linker is from about 1 to about 2 amino acids in length.

In embodiments, the linker is from 1 to 50 amino acids in length. In embodiments, the linker is from 2 to 50 amino acids in length. In embodiments, the linker is from 3 to 50 amino acids in length. In embodiments, the linker is from 4 to 50 amino acids in length. In embodiments, the linker is from 5 to 50 amino acids in length. In embodiments, the linker is from 6 to 50 amino acids in length. In embodiments, the linker is from 7 to 50 amino acids in length. In embodiments, the linker is from 8 to 50 amino acids in length. In embodiments, the linker is from 9 to 50 amino acids in length. In embodiments, the linker is from 10 to 50 amino acids in length. In embodiments, the linker is from 11 to 50 amino acids in length. In embodiments, the linker is from 12 to 50 amino acids in length. In embodiments, the linker is from 13 to 50 amino acids in length. In embodiments, the linker is from 14 to 50 amino acids in length. In embodiments, the linker is from 15 to 50 amino acids in length. In embodiments, the linker is from 20 to 50 amino acids in length. In embodiments, the linker is from 25 to 500 amino acids in length. In embodiments, the linker is from 30 to 500 amino acids in length. In embodiments, the linker is from 35 to 500 amino acids in length. In embodiments, the linker is from 40 to 50 amino acids in length. In embodiments, the linker is from 45 to 50 amino acids in length.

In embodiments, the linker is from 1 to 45 amino acids in length. In embodiments, the linker is from 1 to 40 amino acids in length. In embodiments, the linker is from 1 to 35 amino acids in length. In embodiments, the linker is from 1 to 30 amino acids in length. In embodiments, the linker is from 1 to 25 amino acids in length. In embodiments, the linker is from 1 to 20 amino acids in length. In embodiments, the linker is from 1 to 15 amino acids in length. In embodiments, the linker is from 1 to 14 amino acids in length. In embodiments, the linker is from 1 to 13 amino acids in length. In embodiments, the linker is from 1 to 12 amino acids in length. In embodiments, the linker is from 1 to 11 amino acids in length. In embodiments, the linker is from 1 to 10 amino acids in length. In embodiments, the linker is from 1 to 9 amino acids in length. In embodiments, the linker is from 1 to 8 amino acids in length. In embodiments, the linker is from 1 to 7 amino acids in length. In embodiments, the linker is from 1 to 6 amino acids in length. In embodiments, the linker is from 1 to 5 amino acids in length. In embodiments, the linker is from 1 to 4 amino acids in length. In embodiments, the linker is from 1 to 3 amino acids in length. In embodiments, the linker is from 1 to 2 amino acids in length.

In embodiments, the linker includes the amino acid sequence of SEQ ID NO:2349, SEQ ID NO:2350, SEQ ID NO:2351, SEQ ID NO:2352, SEQ ID NO:2353, or SEQ ID NO: 2354. In embodiments, the linker includes the amino acid sequence of SEQ ID NO:2349. In embodiments, the linker includes the amino acid sequence of SEQ ID NO:2350. In embodiments, the linker includes the amino acid sequence of SEQ ID NO:2351. In embodiments, the linker includes the amino acid sequence of SEQ ID NO:2352. In embodiments, the linker includes the amino acid sequence of SEQ ID NO:2353. In embodiments, the linker includes the amino acid sequence of SEQ ID NO:2354.

In embodiments, the linker is the amino acid sequence of SEQ ID NO:2349, SEQ ID NO: 2350, SEQ ID NO:2351, SEQ ID NO:2352, SEQ ID NO:2353, or SEQ ID NO:2354. In embodiments, the linker is the amino acid sequence of SEQ ID NO:2349. In embodiments, the linker is the amino acid sequence of SEQ ID NO:2350. In embodiments, the linker is the amino acid sequence of SEQ ID NO:2351. In embodiments, the linker is the amino acid sequence of SEQ ID NO:2352. In embodiments, the linker is the amino acid sequence of SEQ ID NO:2353. In embodiments, the linker is the amino acid sequence of SEQ ID NO: 2354.

In embodiments, the peptide modulates the expression level of a target protein relative to a cell not treated with the peptide. In embodiments, the peptide increases the activity of a target protein relative to a cell not treated with the peptide. In embodiments, the peptide decreases the activity of a target protein relative to a cell not treated with the peptide. In embodiments, the peptide increases the activity of a downstream protein of a target protein relative to a cell not treated with the peptide. In embodiments, the peptide decreases the activity of a downstream protein of a target protein relative to a cell not treated with the peptide. In embodiments, the peptide inhibits a protein-to-protein interaction. In embodiments, the peptide inhibits a protein-to-protein interaction in a cell. In embodiments, the peptide inhibits a protein-to-nucleic acid interaction. In embodiments, the peptide inhibits a protein-to-nucleic acid interaction in a cell. In embodiments, the peptide activates a protein-to-protein interaction. In embodiments, the peptide activates a protein-to-protein interaction in a cell. In embodiments, the peptide activates a protein-to-nucleic acid interaction. In embodiments, the peptide activates a protein-to-nucleic acid interaction in a cell.

In embodiments, the peptide incudes a delivery molecule to assist in the targeting of or uptake into a cell. In embodiments, the delivery molecule is a protein transduction domain (PTD). In embodiments, the delivery molecule is a cell-penetrating peptide (CPP). In, embodiments, the delivery molecule is a nanoparticle. In embodiments, the delivery molecule is a liposome. In embodiments, the terms cell-penetrating peptide (CPP) and protein transduction domain (PTD) are used interchangeably herein.

In embodiments, the protein transduction domain (PTD) includes a peptide with high alpha-helicity. In embodiments, the protein transduction domain (PTD) includes one or more basic amino acid residues that are substantially aligned along at least one face of the PTD. In embodiments, the protein transduction domain (PTD) includes a naturally occurring peptide. In embodiments, the protein transduction domain (PTD) includes an unnatural amino acid. In embodiments, the protein transduction domain (PTD) includes an amino acid sequence including a strong alpha helical structure. In embodiments the strong alpha helical structure includes one or more arginine residues in the helical cylinder.

In embodiments, the PTD domain includes a peptide represented by the following general formula: B_{P1}-X_{P1}-X_{P2}-X_{P3}-B_{P2}-X_{P4}-X_{P5}-B_{P3}, wherein B_{P1}, B_{P2}, and B_{P3} are each independently a basic amino acid, the same or different; and X_{P1}, X_{P2}, X_{P3}, X_{P4}, and X_{P5} are each independently an alpha-helix enhancing amino acid, the same or different. In embodiments, the PTD domain is represented by the following general formula: B_{P1}-X_{P1}-X_{P2}-B_{P2}-B_{P3}-X_{P3}-X_{P4}-B_{P4}, wherein B_{P1}, B_{P2}, B_{P3}, and B_{P4} are each independently a basic amino acid, the same or different; and X_{P1}, X_{P2}, X_{P3}, and X_{P4} are each independently an alpha-helix enhancing amino acid the same or different.

In embodiments, the PTD includes basic amino acid residues. In embodiments, the PTD includes a lysine or an arginine. In embodiments, the PTD includes a lysine. In embodiments, the PTD includes an arginine. In embodiments, the PTD further includes a proline. In embodiments, the inclusion of a protein in a PTD introduces “kinks” into the structure of the peptide. In embodiments, the PTD includes the amino acid sequence of SEQ ID NO:41. In embodiments, the PTD is the amino acid sequence of SEQ ID NO:41.

In embodiments, the PTD is cationic. In embodiments, the PTD includes between 7 and 10 amino acids. In embodiments, the PTD includes 5-10 arginine (and/or lysine) residues. In embodiments, the PTD includes 5-10 arginine residues. In embodiments, the PTD includes 5-10 lysine residues. In embodiments, the PTD includes 5-10 arginine (and/or lysine) residues over 5-15 amino acids. In embodiments, the PTD includes 5-10 arginine residues over 5-15 amino acids. In embodiments, the PTD includes 5-10 lysine residues over 5-15 amino acids. In embodiments, the PTD includes the amino acid sequence of SEQ ID NO:42. In embodiments, the PTD is the amino acid sequence of SEQ ID NO:42.

In embodiments, the PTD includes a TAT fragment that includes at least amino acids 49 to 56 of TAT up to about the full-length TAT sequence). In embodiments, a TAT fragment may include one or more amino acid changes sufficient to increase the alpha-helicity of the fragment. In embodiments, the amino acid changes introduced will involve adding a recognized alpha-helix enhancing amino acid. In embodiments, the amino acid changes will involve removing one or more amino acids from the TAT fragment that impede alpha helix formation or stability. In embodiments, the TAT fragment will include at least one amino acid substitution with an alpha-helix enhancing amino acid. In embodiments, the TAT fragment will be made by standard peptide synthesis techniques although recombinant DNA approaches may be used in some cases. In embodiments, the substitution is selected so that at least two basic amino acid residues in the TAT fragment are substantially aligned along at least one face of that TAT fragment. In a more specific embodiment, the substitution is chosen so that at least two basic amino acid residues in the TAT 49-56 sequence are substantially aligned along at least one face of that sequence.

In embodiments, the PTD includes a TAT fragment in which the TAT 49-56 sequence has been modified so that at least two basic amino acids in the sequence are substantially aligned along at least one face of the TAT fragment. In embodiments, the TAT fragments include at least one specified amino acid substitution in at least amino acids 49-56 of TAT which substitution aligns the basic amino acid residues of the 49-56 sequence along at least one face of the segment and typically the TAT 49-56 sequence.

In embodiments, the peptide that can be conjugated to a peptide of the disclosure include, but are not limited to, AntHD, TAT, VP22, cationic prion protein domains, and functional fragments thereof. Not only can these peptides pass through the plasma membrane, but the attachment of other peptide or polypeptides are sufficient to stimulate the cellular uptake of these complexes. Such chimeric peptides/polypeptide are present in a biologically active form within the cytoplasm and nucleus. Characterization of this process

In a particular embodiment, the disclosure therefore provides methods and compositions that combine the use of PTDs, such as TAT and poly-Arg, with a drug-like peptide provided herein to facilitate the uptake of the construct into and/or release within targeted cells. The drug-like peptides provided herein can be delivered into cells using one or more PTDs linked to the drug-like peptide.

In embodiments, the delivery domain that is linked to a drug-like peptide provided herein can be nearly any synthetic or naturally-occurring amino acid sequence which assists in the intracellular delivery of a construct provided herein into targeted cells. For example, delivery of a drug-like peptide (SEQ ID NOs: 1-2327) in accordance with the disclosure can be accomplished by use of a peptide transduction domain, such as an HIV TAT protein or fragment thereof, that is covalently linked to a drug-like peptide of the disclosure. In embodiments, the peptide transduction domain includes an HIV TAT protein or fragment thereof. In embodiments, the peptide transduction domain includes an Antennapedia homeodomain or an HSV VP22 sequence, a N-terminal fragment of a prion protein or suitable transducing fragments thereof such as those known in the art.

The type and size of the PTD will be guided by several parameters including the extent of transfection desired. Typically the PTD will be capable of transfecting at least about 20%, 25%, 50%, 75%, 80% or 90%, 95%, 98% and up to, and including, about 100% of the cells. Transfection efficiency, typically expressed as the percentage of transfected cells, can be determined by several conventional methods.

PTDs will manifest cell entry and exit rates (sometimes referred to as k₁ and k₂, respectively) that favor at least picomolar amounts of a construct provided herein into a targeted cell. The entry and exit rates of the PTD and any cargo can be readily determined or at least approximated by standard kinetic analysis using detectably-labeled fusion molecules. Typically, the ratio of the entry rate to the exit rate will be in the range of between about 5 to about 100 up to about 1000.

Also included are chimeric PTD domains. Such chimeric PTDs include parts of at least two different transducing proteins. For example, chimeric PTDs can be formed by fusing two different TAT fragments, e.g., one from HIV-1 and the other from HIV-2 or one from a prion protein and one from HIV.

Peptide linkers that can be used in the constructs and methods of the disclosure will typically include up to about 20 or 30 amino acids, commonly up to about 10 or 15 amino acids, and still more often from about 1 to 5 amino acids. The linker sequence is generally flexible so as not to hold the fusion molecule in a single rigid conformation. The linker sequence can be used, e.g., to space the PTD domain from a drug-like peptide to be delivered. For example, the peptide linker sequence can be positioned between the peptide transduction domain and the therapeutic drug-like peptide domain, e.g., to provide molecular flexibility. The length of the linker moiety can be chosen to optimize the biological activity of the peptide or polypeptide including, for example, a PTD domain fusion construct and can be determined empirically without undue experimentation. Examples of linker moieties are -Gly-Gly-, GGGGS (SEQ ID NO: SEQ ID NO: 2349), wherein SEQ ID NO:2349 can be repeated 1 or more times, GKSSGSGSESKS (SEQ ID NO:2350), GSTSGSGKSSEGKG (SEQ ID NO:2351), GSTSGSGKSSEGSGSTKG (SEQ ID NO:2352), GSTSGSGKPGSGEGSTKG (SEQ ID NO: 2353), or EGKSSGSGSESKEF (SEQ ID NO:2354). Peptide or polypeptide linking moieties are described, for example, in Huston et al., Proc. Nat'l Acad. Sci. 85:5879, 1988; Whitlow et al., Protein Engineering 6:989, 1993; and Newton et al., Biochemistry 35:545, 1996. Other suitable peptide or polypeptide linkers are those described in U.S. Pat. Nos. 4,751,180 and 4,935,233, which are hereby incorporated by reference.

The amino acid sequences of the various oncogenic and receptor proteins described herein are provided. The drug-like peptides of the disclosure (see SEQ ID NOs: 1-2327), may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, e.g., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. A commonly used N-α-protecting groups include Boc, which is acid labile, and Fmoc, which is base labile.

Materials suitable for use as the solid support are well known to those of skill in the art and include, but are not limited to: halomethyl resins, such as chloromethyl resin or bromomethyl resin; hydroxymethyl resins; phenol resins, such as 4-(α[2,4-dimethoxyphenyl]-Fmoc-aminomethyl) phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid can be first attached to the solid support. The N-α-protecting group can then be removed. The deprotected α-amino group can be coupled to the activated α-carboxylate group of the next N-α-protected amino acid. The process can be repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993), each of which is incorporated herein in their entirety and for all purposes).

Following verification of the coding sequence, a peptide of interest (e.g., a drug-like peptide of the disclosure) can be produced using routine techniques in the field of recombinant molecular biology, relying on the polynucleotide sequences encoding the peptide. The coding sequence of the peptide can be easily deduced using the degeneracy of the genetic code and a codon table.

In embodiments, a peptide or salt thereof can modulate expression level of a target protein. embodiments, a target protein can be implicated in a disease or condition.

In embodiments, a target protein includes an enzyme or fragment thereof. In embodiments, a target protein includes, a kinase, a phosphatase, a signaling peptide, a transcription factor, or any combination thereof. In embodiments, a target protein includes an oxidoreductase, a hydrolase, a transferase, a lyase, an isomerase, or a ligase.

In embodiments, a peptide or salt thereof can modulate a target protein.

Modulation of a target protein by a peptide or salt thereof includes at least a partial inhibition, reduction, or total elimination of activity. In embodiments, modulation includes at least a partial increase in activity. In embodiments, modulation can be achieved by at least partially inhibiting or activating protein to protein interaction. In embodiments, a protein to protein interaction includes a ligand to receptor interaction. In embodiments, a protein to protein interaction includes a regulatory protein complex. In embodiments, a peptide or salt thereof can at least partially reduce a protein to nucleic acid interaction.

In embodiments, modulation of expression level can be determined using an in vitro assay. In embodiments, modulation of activity can be measured relative to an amount of the target protein or activity by a target protein in a cell that has not been treated with a peptide or salt thereof. In embodiments, an assay can be utilized to measure kinase activity or phosphatase activity of a target protein. In embodiments, kinase or phosphatase activity can be determined by evaluating activity of proteins downstream from the target protein. Downstream proteins include proteins that can interact with a target protein directly or indirectly. In embodiments, a downstream protein is at least about 1, 2, 3, 4, 5, 6, 7, 8, 9, or up to 10 proteins removed from the target protein in a pathway of a cell.

Any in vitro assay can be utilized in a method provided herein. In embodiments, an in vitro assay includes a Western blot, PCR, RNA sequencing, Northern blot, qPCR, ELISA, flow cytometry, fluorescence staining, and any combination thereof.

In embodiments, a peptide or salt thereof can produce an at least partial increase or an at least partial decrease of an activity of a downstream protein.

In embodiments, a peptide of salt thereof can have anti-cancer activity. Anti-cancer activity can refer to reduction or elimination of cancer. In embodiments, anti-cancer activity includes killing of a cancer cell. Anti-cancer activity can be determined in vivo or in vitro. In embodiments, a cancer cell, such as a cancer cell line can be utilized. Suitable cancer cells for use in methods provided herein include in vitro cell lines or primary cancer cells. In embodiments, a peptide or salt thereof can modulate an expression level of a target protein. Modulation can be determined utilizing an assay or mouse model to determine a level of killing of a cancer cell.

In embodiments, provided herein can be partial increases or partial decreases of an activity of a target protein. Increases or decreases can refer to at least about a 1-fold, 2-fold, 3-fold, 4-fold, 5-fold, 6-fold, 7-fold, 8-fold, 9-fold, 10-fold, 15-fold, 20-fold, 30-fold, 50-fold, 100-fold, 150-fold, 175-fold, 200-fold, 250-fold, 300-fold, 400-fold, 500-fold, 800-fold, or up to about 1000-fold change in an activity as compared to a comparable method that lacks treatment with a peptide or salt thereof. In embodiments, a partial increase or a partial decrease can refer to about: 1%, 5%, 10%, 20%, 40%, 60%, 80%, 100%, 150%, 200%, 250%, 300%, 350%, 400%, or up to about a 500% increase or decrease in an activity of a target protein. In embodiments, a peptide or salt thereof includes independently Gly, or an amino acid including a C₁-C₁₀ alkyl, a C₁-C₁₀ alkenyl, a C₁-C₁₀ alkynyl, a cycloalkyl, or an alkylcycloalkyl side chain. In embodiments, a peptide includes an amino acid including an aromatic side chain. In embodiments, a peptide includes an amino acid including a side chain that can be at least partially protonated or at least partially deprotonated at a pH of about 7.3. In embodiments, an amino acid of the peptide or salt thereof positioned at an end terminus includes a side chain that can be at least partially deprotonated at a pH of about 7.3. In embodiments, a peptide includes an amino acid including an amide containing side chain. In embodiments, a peptide includes an amino acid including an alcohol or thiol containing side chain. In embodiments, a peptide or salt thereof includes a recombinant peptide.

In embodiments, a peptide can be an engineered peptide. In embodiments, a peptide can be a natural peptide, a synthetic peptide, an artificial peptide, a modified peptide, or any combination thereof. In embodiments, a peptide includes a chemical modification, such as an acetylation, a sulfonation, an amidation, or an esterification. In embodiments, a peptide includes a stapled peptide or salt thereof, a stitched peptide or salt thereof, a macrocyclic peptide or salt thereof, or any combination thereof. In embodiments, a stapled peptide includes a covalent linkage between two amino acid side-chains. In embodiments, a stapled peptide includes an alpha-helix. A stitched peptide or salt thereof includes multiple staples, for example a stitched peptide includes a plurality of covalent linkages between different amino acid side-chains on a peptide. In embodiments, a macrocyclic peptide includes a ring structure, or a bicyclic structure. In embodiments, a macrocyclic peptide includes a head-to-tail, a side-chain-to-side-chain, or both, structure.

In embodiments, a peptide provided herein can further include a linker. A linker can provide desirable flexibility to permit the desired expression, activity and/or conformational positioning of a peptide. A linker can be of any appropriate length and is preferably designed to be sufficiently flexible so as to allow the proper folding and/or function and/or activity of one or both of the domains it connects. In embodiments, a linker can have a length of at least 3, 5, 10, 15, 20, 25, 30, 35, 40, 45, 50, 55, 60, 65, 70, 75, 80, 85, 90, 95, or 100 residues. In embodiments, a linker has a length from about 0 to 200 residues, from about 10 to 190 residues, from about 20 to 180 residues, from about 30 to 170 residues, from about 40 to 160 residues, from about 50 to 150 residues, from about 60 to 140 residues, from about 70 to 130 residues, from about 80 to 120 residues, or from about 90 to 110 residues. In embodiments, a linker includes an endogenous protein sequence. In embodiments, a linker sequence includes glycine, alanine, or serine amino acid residues, or any combination of glycine, alanine, and serine amino acid residues. In embodiments, a linker can contain motifs, e.g., multiple or repeating motifs, of glycine-serine, glycine-glycine-serine, glycine-glycine-glycine-glycine-serine, glycine-glycine-serine-glycine, or serine-glycine-glycine-glycine. In embodiments, a linker sequence can include any naturally occurring amino acids, non-naturally occurring amino acids, or combinations thereof. In embodiments, a linker can be a cleavable linker. In embodiments, a linker can at least in part be resistant to cleavage. In embodiments, a resistant cleavage linker includes a thioether linker, a maleimide alkane linker, a maleimide cyclohexane linker, or any combination thereof. In embodiments, a cleavable linker includes an enzymatically cleavable linker, a chemically cleavable linker, or both. A cleavable linkage includes an acid-labile linker, a reducible linker, a disulfide-linker, a hydrazone linker, a peptide linker, or any combination thereof. In embodiments, a cleavable linker can be linked to an antibody or a fragment thereof. In embodiments, a cleavable linker can be linked to a cell penetrating peptide. In embodiments, a peptide can be directly or indirectly linked to a cell penetrating peptide. A cell penetrating peptide can be a short polypeptide that can allow for increased uptake of a subject composition into a cell. A cell penetrating peptide can be cationic, amphipathic, hydrophobic, and combinations thereof. In embodiments, a cell penetrating peptide includes a TAT peptide, a MPG peptide, a Pep-1 peptide, a KALA peptide, a SV40 NLS peptide, an Arg polypeptide, a TfR targeting peptide, a rabies virus glycoprotein, or a penetratin peptide. A cell penetrating peptide can be of any length. In embodiments, a cell penetrating peptide can be about 3 amino acids to about 50 amino acids long, about 5 amino acids to about 15 amino acids long, about 10 amino acids to about 25 amino acids long, about 20 amino acids to about 30 amino acids long, or about 30 amino acids to about 50 amino acids long. In embodiments, a cell penetrating peptide includes a peptide that includes L-amino acids, D-amino acids, both L and D amino acids, and non-natural amino acids. In embodiments, a cell penetrating peptide includes a cyclic peptide. In embodiments, a cell penetrating peptide includes a poly-arginine stretch.

### Nucleic Acid Compositions

The compositions provided herein include nucleic acid molecules encoding the peptides or portions thereof provided herein including embodiments thereof. The peptides encoded by the isolated nucleic acid provided herein are described in detail throughout this application (including the description above and in the examples section). Thus, in an aspect is provided an isolated nucleic acid encoding the peptide provided herein including embodiments thereof.

In embodiments, the isolated nucleic acid is linked to an expression control sequence. In embodiments, the expression control sequence is a promoter. In embodiments, the expression control sequence is an enhancer.

In embodiments, the isolated nucleic acid can be packaged into an expression vector. In embodiments, the expression vector is a viral vector. In embodiments, the viral vector is replication competent. In embodiments, the viral vector is replication defective.

### Expression Vector Compositions

The compositions provided herein include expression vectors including a nucleic acid encoding peptides or portions thereof provided herein including embodiments thereof. The peptides encoded by the isolated nucleic acid provided herein are described in detail throughout this application (including the description above and in the examples section). Thus, in an aspect is provided an expression vector including the isolated nucleic acid provided herein including embodiments thereof.

In embodiments, the expression vector is a viral vector. In embodiments, the viral vector is an Adeno-associated viral (AAV) vector, an Adenovirus vector, or a lentiviral vector. In embodiments, the viral vector is an Adeno-associated viral (AAV) vector. In embodiments, the viral vector is an Adenovirus vector. In embodiments, the viral vector is a lentiviral vector.

In embodiments, the screening method provided herein includes an expression vector to deliver a gene fragment to a cell. In embodiments, the expression vector is a viral vector. In embodiments, the gene fragments are packaged into viral vectors for delivery of gene fragments to target cells for overexpression. Examples of viral vectors, include, but are not limited to, recombinant retroviral vectors, adenoviral vectors, adeno-associated viral vector, alphaviral vectors, and lentiviral vectors.

In embodiments, the viral vector is a lentiviral vector. Because lentiviruses integrate into the genome, the viral integrant serves as a tag for readout of which sgRNA construct can be delivered to a particular cell. Lentivirus is unique in its ability to infect non-dividing cells, and therefore has a wider range of potential applications. In embodiments, the lentiviral genome in the form of RNA is reverse-transcribed when the virus enters the cell to produce DNA, which is then inserted into the genome at a position determined by the viral integrase enzyme. For safety reasons lentiviral vectors never carry the genes required for their replication.

In embodiments, the viral vector is an adeno-associated virus (AAV). AAV is a tiny non-enveloped virus having a 25 nm capsid. In embodiments, no disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA can be limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. For example, AAV9 has been shown to efficiently cross the blood-brain barrier. Moreover, the AAV capsid can be genetically engineered to increase transduction efficient and selectivity, e.g., biotinylated AAV vectors, directed molecular evolution, self-complementary AAV genomes and so on. Modified AAV have also been described, including AAV based on ancestral sequences; see, e.g., U.S. Pat. No. 7,906,111; WO/2005/033321; WO2008027084, WO2014124282; WO2015054653; and WO2007127264. Other modified AAVs that have been described include chimeric nanoparticles (ChNPs) that have an AAV core that expresses a transgene that is surrounded by layer(s) of acid labile polymers that have embedded antisense oligonucleotides (e.g., see Hong et al., ACS Nano 10:8705-8716 (2016)) and Cho et al., Biomaterials 2012, 33, 3316-3323). The compositions and methods provided herein, In embodiments, provide a platform technology, and as such the composition and methods provided herein can be used with all known AAVs, including the modified AAVs described in the literature, such as ChNPs.

In embodiments, the viral vector is a retrovirus vector. These retrovirus vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for viral gene therapy, and defective retroviruses are characterized for use in gene transfer for viral gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al, eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include Ψ&iρ, Ψ&ε, Ψ2 and ΨAπι. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

In embodiments, the viral vector is a adenovirus-derived vector. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle can be relatively stable and amenable to purification and concentration, and as above, can be modified so as to affect the spectrum of infectivity. In embodiments, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA can be large (up to 8 kilobases) relative to other gene delivery vectors (Berkner et al., supra; Haj-Ahmand and Graham, J. Virol. 57:267 (1986).

In embodiments, the viral vector is an alphavirus. Alphaviruses are enveloped single stranded RNA viruses that have a broad host range, and when used in the methods provided herein alphaviruses can provide high-level transient gene fragment expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases. See, e.g., Lundstrom, Viruses. 2009 June; 1 (1): 13-25; Lundstrom, Viruses. 2014 June; 6 (6): 2392-2415; Lundstrom, Curr Gene Ther. 2001 May; 1 (1): 19-29; Rayner et al., Rev Med Virol. 2002 Sep.-Oct. 12 (5): 279-96.

In embodiments, the viral vector is a gamma-retroviral vector. In embodiments, the gamma-retroviral vector is replication competent or defective. In embodiments, the gamma-retroviral vector is replication competent. In embodiments, the gamma-retroviral vector is replication defective. In embodiments, the gamma-retroviral vector is murine leukemia virus (MLV). Typical constructs include viruses with a viral genome including viral genes (e.g., gag, pol, env) having an expression cassette located in the LTRs or downstream of the envelope gene.

To obtain high level expression of a nucleic acid encoding a peptide of interest, one typically subclones the polynucleotide coding sequence into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al. Bacterial expression systems for expressing recombinant polypeptides are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. In embodiments, the promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the desired peptide in host cells. A typical expression cassette thus contains a promoter operably linked to the nucleic acid sequence encoding the peptide and signals required for efficient polyadenylation of the transcript, ribosome binding sites, and translation termination. The nucleic acid sequence encoding the desired peptide is typically linked to a cleavable signal peptide sequence to promote secretion of the recombinant polypeptide by the transformed cell. Such signal peptides include, among others, the signal peptides from tissue plasminogen activator, insulin, and neuron growth factor, and juvenile hormone esterase of Heliothis virescens. If, however, a recombinant polypeptide is intended to be expressed on the host cell surface, an appropriate anchoring sequence is used in concert with the coding sequence. Additional elements of the cassette may include enhancers and, if genomic DNA is used as the structural gene, introns with functional splice donor and acceptor sites.

In addition to a promoter sequence, the expression cassette should also contain a transcription termination region downstream of the structural peptide coding sequence to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the desired peptide under the direction of the polyhedrin promoter or other strong baculovirus promoters.

When periplasmic expression of a recombinant polypeptide is desired, the expression vector further includes a sequence encoding a secretion signal, such as the E. coli OppA (Periplasmic Oligopeptide Binding Protein) secretion signal or a modified version thereof, which is directly connected to 5′ of the coding sequence of the peptide to be expressed. This signal sequence directs the recombinant peptide produced in the cytoplasm through the cell membrane into the periplasmic space. The expression vector may further include a coding sequence for signal peptidase 1, which is capable of enzymatically cleaving the signal sequence when the recombinant peptide is entering the periplasmic space. More detailed description for periplasmic production of a recombinant protein can be found in, e.g., Gray et al., Gene 39:247-254 (1985), U.S. Pat. Nos. 6,160,089 and 6,436,674.

Standard transfection methods are used to produce bacterial, mammalian, yeast, insect, or plant cell lines that express large quantities of a recombinant peptide or polypeptide, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell. In embodiments, it is only necessary that the particular genetic engineering procedure used be capable of successfully introducing at least one gene into the host cell capable of expressing the recombinant polypeptide.

In embodiments, once the expression of a recombinant peptide in transfected host cells is confirmed, e.g., by an immunological assay, activity assay or sequencing, the host cells are then cultured in an appropriate scale for the purpose of purifying the recombinant peptide.

When desired polypeptides are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, and will be apparent to those of skill in the art.

For delivery to a cell or organism, a nucleic acid encoding a drug-like peptide of the disclosure can be incorporated into a vector. Examples of vectors used for such purposes include expression plasmids capable of directing the expression of the drug-like peptide in the target cell. In other instances, the vector can be a viral vector system wherein the nucleic acid encoding the drug-like peptide is incorporated into a viral genome that is capable of transfecting the target cell.

As used herein, “gene delivery system” refers to any means for the delivery of a nucleic acid encoding a drug-like peptide of the disclosure to a target cell. Viral vector systems useful in the introduction and expression of a nucleic acid include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the nucleic acid is inserted into such vectors to allow packaging of the gene construct, typically with accompanying viral DNA, followed by infection of a sensitive host cell and expression of the gene of interest.

Similarly, viral envelopes used for packaging gene constructs that include the nucleic acid can be modified by the addition of receptor ligands or antibodies specific for a receptor to permit receptor-mediated endocytosis into specific cells.

Retroviral vectors may also be useful for introducing the nucleic acid into target cells or organisms. Retroviral vectors are produced by genetically manipulating retroviruses. The viral genome of retroviruses is RNA. Upon infection, this genomic RNA is reverse transcribed into a DNA copy that is integrated into the chromosomal DNA of transduced cells with a high degree of stability and efficiency. The integrated DNA copy is referred to as a provirus and is inherited by daughter cells as is any other gene. The wild type retroviral genome and the proviral DNA include three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943, each of which is incorporated herein in their entirety and for all purposes.

The retroviral vector particles are prepared by recombinantly inserting the desired nucleic acid sequence encoding a drug-like peptide of the disclosure into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence.

Delivery of a drug-like peptide of the disclosure can be achieved by contacting a cell with a nucleic acid construct or using direct delivery of the drug-like peptide or fusion peptide. In a particular embodiment, a drug-like peptide of the disclosure (e.g., a therapeutic peptide) can be formulated with various carriers, dispersion agents and the like, as are described more fully elsewhere herein.

### Pharmaceutical Compositions

The peptide compositions and nucleic acid compositions provided herein can be included in a pharmaceutical composition. The peptides and nucleic acids provided herein are described in detail throughout this application (including the description above and in the examples section). Thus, in an aspect is provided a pharmaceutical composition including: (i) the peptide provided herein including embodiments thereof, the nucleic acid provided herein including embodiments thereof, or the expression vector provided herein including embodiments thereof; and (ii) a pharmaceutically acceptable excipient.

A pharmaceutical composition according to the disclosure can be prepared to include a drug-like peptide as provided herein, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, and The National Formulary, 30th ed., the contents of which are hereby incorporated by reference. The pH and exact concentration of the various components of the pharmaceutical composition are adjusted according to routine skills in the art. See Goodman and Gilman's, The Pharmacological Basis for Therapeutics.

The pharmaceutical compositions according to the disclosure may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, transdermal application, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically, the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and

formulations. Thus, a pharmaceutically acceptable carrier can include solvents, dispersion media, coatings, antibacterial and antifungal agents, isotonic and absorption delaying agents, and the like. In embodiments, the use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

In embodiments, it can be especially advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition can be calculated to produce the desired therapeutic effect in association with the required pharmaceutical carrier. The specification for the dosage unit forms of the disclosure are related to the characteristics of the pharmaceutical composition and the particular therapeutic effect to be achieve. The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

For topical formulations, the base composition can be prepared with any solvent system, such as those Generally Regarded as Safe (GRAS) by the U.S. Food & Drug Administration (FDA). GRAS solvent systems include many short chain hydrocarbons, such as butane, propane, n-butane, or a mixture thereof, as the delivery vehicle, which are approved by the FDA for topical use. The topical compositions can be formulated using any dermatologically acceptable carrier. Exemplary carriers include a solid carrier, such as alumina, clay, microcrystalline cellulose, silica, or talc; and/or a liquid carrier, such as an alcohol, a glycol, or a water-alcohol/glycol blend. The compounds may also be administered in liposomal formulations that allow compounds to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061. Examples of other appropriate vehicles are described in U.S. Pat. Nos. 4,877,805, 4,980,378, 5,082,866, 6,118,020 and EP Publication No. 0586106A1. Suitable vehicles of the disclosure may also include mineral oil, petrolatum, polydecene, stearic acid, isopropyl myristate, polyoxyl 40 stearate, stearyl alcohol, or vegetable oil.

Topical compositions can be provided in any useful form. For example, the compositions of the disclosure may be formulated as solutions, emulsions (including microemulsions), suspensions, creams, foams, lotions, gels, powders, balm, or other typical solid, semi-solid, or liquid compositions used for application to the skin or other tissues where the compositions may be used. Such compositions may contain other ingredients typically used in such products, such as colorants, fragrances, thickeners, antimicrobials, solvents, surfactants, detergents, gelling agents, antioxidants, fillers, dyestuffs, viscosity-controlling agents, preservatives, humectants, emollients (e.g., natural or synthetic oils, hydrocarbon oils, waxes, or silicones), hydration agents, chelating agents, demulcents, solubilizing excipients, adjuvants, dispersants, skin penetration enhancers, plasticizing agents, preservatives, stabilizers, demulsifiers, wetting agents, sunscreens, emulsifiers, moisturizers, astringents, deodorants, and optionally including anesthetics, anti-itch actives, botanical extracts, conditioning agents, darkening or lightening agents, glitter, humectants, mica, minerals, polyphenols, silicones or derivatives thereof, sunblocks, vitamins, and phytomedicinals.

In some formulations, the composition can be formulated for ocular application. For example, a pharmaceutical formulation for ocular application can include a polynucleotide construct as described herein in an amount that is, e.g., up to 99% by weight mixed with a physiologically acceptable ophthalmic carrier medium such as water, buffer, saline, glycine, hyaluronic acid, mannitol, and the like. For ophthalmic delivery, a polynucleotide construct as described herein may be combined with ophthalmologically acceptable preservatives, co-solvents, surfactants, viscosity enhancers, penetration enhancers, buffers, sodium chloride, or water to form an aqueous, sterile ophthalmic suspension or solution.

Ophthalmic solution formulations may be prepared by dissolving the polynucleotide construct in a physiologically acceptable isotonic aqueous buffer. Further, the ophthalmic solution may include an ophthalmologically acceptable surfactant to assist in dissolving the inhibitor. Viscosity building agents, such as hydroxymethyl cellulose, hydroxyethyl cellulose, methylcellulose, polyvinylpyrrolidone, or the like may be added to the compositions of the disclosure to improve the retention of the compound.

Topical compositions can be delivered to the surface of the eye, e.g., one to four times per day, or on an extended delivery schedule such as daily, weekly, bi-weekly, monthly, or longer, according to the routine discretion of a skilled clinician. The pH of the formulation can range from about pH 4-9, or about pH 4.5 to pH 7.4.

For nucleic acid constructs of the disclosure, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

In embodiments, a pharmaceutical composition includes a nucleic acid at least partially encoding a peptide described herein. In embodiments, a nucleic acid can at least partially encode a peptide that can have at least about: 70%, 75%, 80%, 85%, 90%, 95% or 99% sequence identity to a polypeptide having a sequence set forth in any one of SEQ ID NOs: 1-2327. In embodiments, the peptide may not include more than: about 10 amino acids to about 60 amino acids, about 10 amino acids to about 20 amino acids, about 20 amino acids to about 30 amino acids, about 30 amino acids to about 40 amino acids, about 40 amino acids to about 50 amino acids, or about 50 amino acids to about 60 amino acids. In embodiments, a nucleic acid includes a pharmaceutical composition in unit dose form. In embodiments, a nucleic acid includes DNA, RNA, or both. In embodiments, a nucleic acid can be circular, such as a plasmid. A nucleic acid can be single stranded, double stranded, or both.

In embodiments, a pharmaceutical composition includes a vector. A vector includes or encodes a peptide described herein, for example a peptide for use in a pharmaceutical composition. In embodiments, a vector includes a nucleic acid. In embodiments, a vector includes a polypeptide coat. In embodiments, a vector can be a viral vector, a virus-like particle. In embodiments, the vector includes an RNA viral vector which can include but may not be limited to a retrovirus, lentivirus, coronavirus, alphavirus, flavivirus, rhabdovirus, morbillivirus, picornavirus, coxsackievirus, or picornavirus or portions of any of these, or fragments of any of these, or any combination thereof. In embodiments, a vector includes a lentiviral vector. In embodiments, a vector includes a DNA viral vector which can include but may not be limited to an adeno-associated viral (AAV) vector, adenovirus, hybrid adenoviral system, hepadnavirus, parvovirus, papillomavirus, polyomavirus, herpesvirus, poxvirus, a portion of any of these, or a fragment of any of these, or any combination thereof. In embodiments, a vector includes an AAV vector. An AAV vector, can be of any serotype. In embodiments, an AAV vector is of a serotype selected from any one of: AAV1, AAV2, AAV3, AAV4, AAV5, AAV6, AAV7, AAV8, AAV9, AAV10, AAV11, AAV12, AAVDJ, variants thereof, and any combination thereof. In embodiments, a vector is of AAV2, AAV5, AAV9, or combinations thereof. Provided herein are also modified vectors that include mutations or modifications of components such as modified REP, CAP, ITRs, or combinations thereof. In embodiments, chimeras of AAV vectors are also employed in methods and compositions provided herein.

Compositions provided herein can be delivered via any means. In embodiments, composition is delivered via a vector. In embodiments, a vector includes a polypeptide coat. In embodiments, a vector includes a liposome, a nanoparticle, a microparticle, or any combination thereof. In embodiments, a liposome can include but may not be limited to a unilamellar liposome, multilamellar liposome, archaeosome, noisome, novasome, cryptosome, emulsome, vesosome, or a derivative of any of these, or any combination thereof. In embodiments, a vector includes a nanoparticle. A nanoparticle, can include but may not be limited to a biopolymeric nanoparticle, an alginate nanoparticle, a xanthan gum nanoparticle, a cellulose nanoparticle, a dendrimer, a polymeric micelle, polyplexed, an inorganic nanoparticle, a nanocrystal, a metallic nanoparticle, a quantum dot, a protein nanoparticle, a polysaccharide nanoparticle, or a derivative of any of these, or any combination thereof.

In embodiments, a pharmaceutical composition can be in unit dose form. In embodiments, compositions provided herein can be in unit dose forms or multiple-dose forms. Unit dose forms, as used herein, can refer to physically discrete units suitable for administration to human or non-human subjects (e.g., animals). In embodiments, a nucleic acid or a peptide is present in a composition in a range of from about 1 mg to about 2000 mg; from about 5 mg to about 1000 mg, from about 10 mg to about 25 mg to 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg. In embodiments, a dose of a composition provided herein is at least about or at most about: 10,000, 15,000, 20,000, 22,000, 24,000, 25,000, 30,000, 40,000, 50,000, 60,000, 70,000, 80,000, 90,000, 100,000, 125,000, 150,000, 200,000, or 500,000 units/kg body weight. In embodiments, a therapeutically effective dose is at most about 10,000, 15,000, 20,000, 22,000, 24,000, 25,000, 30,000, 40,000, 50,000, 60,000, 70,000, 80,000, 90,000, 100,000, 125,000, 150,000, 200,000, or 500,000 units/kg body weight. Any one of the described dosages can be determined using the 95% CI for plasma volumes and body surface areas in males and females and an 10×IC50 (to achieve an about IC90 concentration, based on the shapes of a killing curves). In embodiments, a dose range is from about 17-440 micromoles/m\hat{ }2 (body surface area) per peptide. In embodiments, a dosage is at least or at most about: 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, or up to about 500 micromoles/m\hat{ }2 (body surface area) per peptide.

In embodiments, unit dose forms can be packaged individually. Each unit dose can contain a predetermined quantity of an active ingredient(s) that can be sufficient to produce the desired therapeutic effect in association with pharmaceutical carriers, diluents, excipients, or any combination thereof. Examples of unit dose forms can include, ampules, syringes, and individually packaged tablets and capsules. In embodiments, a unit dose form can be included in a disposable syringe. In embodiments, unit-dosage forms can be administered in fractions or multiples thereof. A multiple-dose form can be a plurality of identical unit dose forms packaged in a single container, which can be administered in segregated a unit dose form. Examples of a multiple-dose form can include vials, bottles of tablets or capsules, or bottles of pints or gallons. In embodiments, a multiple-dose form includes the same pharmaceutically active agents. In embodiments, a multiple-dose form includes different pharmaceutically active agents.

In embodiments, a pharmaceutical composition includes: a peptide or salt thereof; and at least one of: an excipient, a diluent, or a carrier.

Administration or application of a composition provided herein can be performed on any animal, such as a human, a non-human primate, a pet (e.g., a dog, a cat), or a farm animal (a horse, a cow, a goat, a pig). In embodiments, administration or application of a composition provided herein can be performed on a human. In embodiments, a human can be from about 1 day to about 1 month old, from about 1 month to about 12 months old, from about 1 year to about 7 years old, from about 5 years to about 25 years old, from about 20 years to about 50 years old, from about 45 years to about 80 years old, or from about 75 years to about 130 years old.

Compositions described herein can be administered before, during, or after the occurrence of a disease or condition, and the timing of administering a composition can vary. For example, a pharmaceutical compositions can be used as a prophylactic and can be administered continuously to subjects with a propensity to conditions or diseases in order to prevent the occurrence of the disease or condition. Pharmaceutical compositions can be administered to a subject during or as soon as possible after the onset of the symptoms. The administration of the molecules can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein, such as by oral administration, topical administration, intravenous administration, inhalation administration, injection, catheterization, gastrostomy tube administration, intraosseous administration, ocular administration, otic administration, transdermal administration, oral administration, rectal administration, nasal administration, intravaginal administration, intracavernous administration, transurethral administration, sublingual administration, or a combination thereof. A composition can be administered as soon as is practicable after the onset of a disease or condition is detected or suspected, and for a length of time necessary for the treatment of the disease, such as, for example, from about 1 month to about 3 months. The length of treatment can vary for each subject. In embodiments, a method can further include administering a second therapy in a therapeutically effective amount. A second therapy can be administered concurrently or consecutively to provided compositions.

Administration or application of a composition provided herein can be performed for a duration of at least about at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000 days consecutive or nonconsecutive days. In embodiments, the composition can be administered for life. In embodiments, administration or application of the composition described herein can be from about 1 to about 30 days, from about 1 to about 60 days, from about 1 to about 90 days, from about 1 to about 300 days, from about 1 to about 3000 days, from about 30 days to about 90 days, from about 60 days to about 900 days, from about 30 days to about 900 days, or from about 90 days to about 1500 days. In embodiments, administration or application of the composition described herein can be from: about 1 week to about 5 weeks, about 1 month to about 12 months, about 1 year to about 3 years, about 2 years to about 8 years, about 3 years to about 10 years, about 10 years to about 50 years, about 15 years to about 40 years, about 25 years to about 100 years, about 30 years to about 75 years, about 60 years to about 110 years, or about 50 years to about 130 years.

Administration or application of a composition provided herein can be performed for a duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or for life.

Administration can be performed repeatedly over a lifetime of a subject, such as once a day, once a week, or once a month for the lifetime of a subject. Administration can be performed repeatedly over a substantial portion of a subject's life, such as once a day, once a week, or once a month for at least about: 1 year, 5 years, 10 years, 15 years, 20 years, 25 years, 30 years, or more.

Administration or application of composition provided herein can be performed at least about: 1, 2, 3, 4, 5, 6, 7, 8, 9, 10, 11, 12, 13, 14, 15, 16, 17, 18, 19, 20, 21, 22, 23, or 24 times a in a 24-hour period. In embodiments, administration or application of a composition provided herein can be performed continuously throughout a 24-hour period. In embodiments, administration or application of composition provided herein can be performed at least about: 1, 2, 3, 4, 5, 6, 7, 8, 9, 10, 11, 12, 13, 14, 15, 16, 17, 18, 19, 20, or 21 times a week. In embodiments, administration or application of a composition provided herein can be performed at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or more times a month. In embodiments, a composition can be administered as a single dose or as divided doses. For example, administration of a capsule, or a tablet an include administration of more than one capsules or tablets. In embodiments, the compositions described herein can be administered at a first time point and a second time point. In embodiments, a composition can be administered such that a first administration can be administered before the other with a difference in administration time of about: 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

Administration of a pharmaceutical composition described herein can be administered in one dose, continuously or intermittently throughout the course of treatment. Methods of determining the most effective means and dosage of administration can be determined by a physician or another medical professional and can vary with the composition used for therapy, the purpose of the therapy, and the subject being treated. For example, depending on the age and size of a subject, an appropriate dosage can be calculated. Additionally, an administration can be delivered locally or systemically depending on disease location. In embodiments, compositions provided herein can be delivered via a vector or without a vector, for example as a naked composition. Single or multiple administrations can be carried out with the dose level and pattern being selected by the treating physician. Suitable dosage formulations and methods of administering the agents can be known in the art. Routes of administration can also be determined and method of determining the most effective routes of administration can be determined by a physician or another medial professional and can vary with the composition used for treatment, the purpose of the treatment, the health condition or disease stage of the subject being treated, and target cell or tissue. Non-limiting examples of routes of administration include oral administration, nasal administration, injection, and topical application.

Administration can refer to methods that can be used to enable the delivery of a pharmaceutical composition described herein (e.g. a peptide) to the desired site of biological action. For example, a nucleic acid encoding for a peptide described herein can be included in a viral vector and can be administered by intravenous administration. Administration provided herein to an area in need of treatment or therapy can be achieved by, for example, and not by way of limitation, oral administration, topical administration, intravenous administration, inhalation administration, or any combination thereof. In embodiments, delivery can include injection, catheterization, gastrostomy tube administration, intraosseous administration, ocular administration, otic administration, transdermal administration, oral administration, rectal administration, nasal administration, intravaginal administration, intracavernous administration, transurethral administration, sublingual administration, or a combination thereof. Delivery can include direct application to the affected tissue or region of the body. In embodiments, topical administration includes administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin.

Delivery can include a parenchymal injection, an intra-thecal injection, an intraventricular injection, or an intra-cisternal injection. A composition provided herein can be administered by any method. A method of administration can be by intraarterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In embodiments, delivery can be from a device. In embodiments, delivery can be administered by a pump, an infusion pump, or a combination thereof. In embodiments, delivery can be by an enema, an eye drop, a nasal spray, or any combination thereof. In embodiments, a subject can administer the composition in the absence of supervision. In embodiments, a subject can administer the composition under the supervision of a medical professional (e.g., a physician, nurse, physician's assistant, orderly, hospice worker, etc.). In embodiments, a medical professional can administer the composition.

In embodiments, administering can be oral ingestion. In embodiments, delivery can be a capsule or a tablet. Oral ingestion delivery includes a tea, an elixir, a food, a drink, a beverage, a syrup, a liquid, a gel, a capsule, a tablet, an oil, a tincture, or any combination thereof. In embodiments, a food can be a medical food. In embodiments, a capsule includes hydroxymethylcellulose. In embodiments, a capsule includes a gelatin, hydroxypropylmethyl cellulose, pullulan, or any combination thereof. In embodiments, capsules includes a coating, for example, an enteric coating. In embodiments, a capsule includes a vegetarian product or a vegan product such as a hypromellose capsule. In embodiments, delivery includes inhalation by an inhaler, a diffuser, a nebulizer, a vaporizer, or a combination thereof.

### Kits

In an aspect is provided herein a kit including the peptide provided herein including embodiments thereof, the nucleic acid provided herein including embodiments thereof, the expression vector provided herein including embodiments thereof, the pharmaceutical composition provided herein including embodiments thereof, or a combination thereof. In embodiments, the kit further includes instructions. In embodiments, the instructions recite the methods of using a pharmaceutical composition described herein, a nucleic acid described herein, a peptide described herein, the vector described herein or any combination thereof. In embodiments, the kit includes instructions for administration to a subject in need thereof. In embodiments, the kit includes a container, such as a plastic, a glass, or a metal container.

### Methods of Treatment

The peptides provided herein including embodiments thereof may be used as a therapeutic for treating a disease or disorder in a subject in need thereof. Thus, in an aspect is provided a method of treating cancer in a subject in need thereof, the method including administering to the subject a therapeutically effective amount of the peptide provided herein including embodiments thereof, thereby treating the cancer in the subject.

In embodiments, the method further includes administering an anticancer therapeutic.

In embodiments, the method includes two or more peptides provided herein including embodiments thereof.

In another aspect is provided a method for treating a disease or condition in a subject in need thereof, the method including administering to the subject a therapeutically effective amount of the peptide provided herein including embodiments thereof, thereby treating the disease or condition in the subject.

The disclosure provides methods for treating a subject with a cancer or suspected of having a cancer including administering a drug-like peptide of the disclosure including a therapeutically effective amount of one or more drug-like peptide and optionally one or more anticancer agents provided herein. A therapeutically effective amount can be measured as the amount sufficient to prevent cancer cells from dividing, metastasizing and/or growing, ultimately killing the cancer cells or reducing the metastatic potential of the cancer cell. Generally, the optimal dosage of will depend upon the type and stage of the cancer and factors such as the weight, sex, and condition of the subject. Nonetheless, suitable dosages can readily be determined by one skilled in the art. Typically, dosages used in vitro may provide useful guidance in the amounts useful for in situ administration of the pharmaceutical composition, and animal models may be used to determine effective dosages for treatment of specific cancers. Various considerations are described, e.g., in Langer, Science, 249:1527, (1990); Gilman et al. (eds.) (1990), each of which is herein incorporated by reference. Typically, a suitable dosage can be 1 to 1000 mg/kg body weight, e.g., 10 to 500 mg/kg body weight. In a particular embodiment, a drug-like peptide provided herein can be administered at dosage of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 100 mg/kg, or a range that includes or is between any two of the foregoing dosages, including fractional dosages thereof.

Examples, of anticancer agents that can be used with the drug-like peptides provided herein include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and tiimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammall and calicheamicin omegall; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitiaerine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2 2″-trichlorotiiethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® (docetaxel) (Rhone-Poulenc Rorer, Antony, France); chloranbucil; GEMZAR® (gemcitabine); 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids such as retinoic acid; capecitabine; leucovorin (LV); irenotecan; adrenocortical suppressant; adrenocorticosteroids; progestins; estrogens; androgens; gonadotropin-releasing hormone analogs; and pharmaceutically acceptable salts, acids or derivatives of any of the above. Also included anticancer agents are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4 (5)-imidazoles, aminoglutethimide, MEGASE® megestrol acetate, AROMASL® exemestane, formestanie, fadrozole, RIVISOR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rJL-2; LURTOTECAN® topoisomerase 1 inhibitor; ABARELLX® rmRH; antibodies such as trastuzumab and pharmaceutically acceptable salts, acids or derivatives of any of the above. In a particular embodiment, the disclosure provides for combined therapy including one or more drug-like peptides provided herein used in combination with a tyrosine kinase inhibitor (TKI). Examples of protein kinase inhibitors, include but are not limited to, adavosertib, afatinib, axitinib, bosutinib, cetuximab, cobimetinib, crizotinib, cabozantinib, dasatinib, entrectinib, erdafitinib, erlotinib, fostamatinib, gefitinib, ibrutinib, imatinib, lapatinib, lenvatinib, mubritinib, nilotinib, pazopanib, pegaptanib, ruxolitinib, sorafenib, sunitinib, SU6656, vandetanib, and vemurafenib.

In embodiments, the peptide provided herein including embodiments thereof can be used in a combination therapy. In embodiments, the method includes the peptide provided herein including embodiments thereof and an angiogenesis inhibitor. Examples of angiogenesis inhibitors, include but are not limited to, axitinib, bevacizumab, cabozantinib, everolimus, lenalidomide, lenvatinib mesylate, pazopanib, ramucirumab, regorafenib, sorafenib, sunitinib, thalidomide, vandetanib, and Ziv-aflibercept. In another embodiment, the disclosure provides for combined therapy including one or more drug-like peptides provided herein used in combination with a PARP inhibitor. Examples of PARP inhibitors, include but are not limited to, olaparib, niraparib, rucaparib, and talzoparib. The anticancer agent may be administered, by a route and in an amount commonly used therefore, simultaneously (at the same time or in the same formulation) or sequentially with a drug-like peptide as provided herein. When a drug-like peptide as provided herein is used contemporaneously with one or more anticancer agents, a pharmaceutical composition containing the one or more anticancer agents in addition to a drug-like peptide provided herein may be utilized but may not be required. Accordingly, the pharmaceutical compositions provided herein include those that also contain one or more anticancer agents in addition to a drug-like peptide provided herein.

In embodiments, the disease or condition includes cancer. In embodiments, the cancer includes a sarcoma, a carcinoma, a melanoma, a lymphoma, a leukemia, a blastoma, a germ cell tumor, a myeloma, or any combination thereof. In embodiments, cancer includes a thyroid cancer, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, uterine cancer (e.g. uterine sarcoma), vaginal cancer, vulvar cancer, or Waldenstrom's macroglobulinemia.

In embodiments, a cancer can include a hyperproliferative disorder. Hyperproliferative disorders can include but may not be limited to cancers, hyperplasia, or neoplasia. In embodiments, the hyperproliferative cancer can be breast cancer such as a ductal carcinoma in duct tissue of a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epithelioid carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which may be divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which may be a skin condition that sometimes develops into squamous cell carcinoma; eye retinoblastoma; cutaneous or intraocular (eye) melanoma; primary liver cancer (cancer that begins in the liver); kidney cancer; autoimmune deficiency syndrome (AIDS)-related lymphoma such as diffuse large B-cell lymphoma, B-cell immunoblastic lymphoma and small noncleaved cell lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer; central nervous system (CNS) cancers such as primary brain tumor, which includes gliomas (astrocytoma, anaplastic astrocytoma, or glioblastoma multiforme), oligodendrogliomas, ependymomas, meningiomas, lymphomas, schwannomas, and medulloblastomas; peripheral nervous system (PNS) cancers such as acoustic neuromas and malignant peripheral nerve sheath tumors (MPNST) including neurofibromas and schwannomas, malignant fibrous cytomas, malignant fibrous histiocytomas, malignant meningiomas, malignant mesotheliomas, and malignant mixed Müllerian tumors; oral cavity and oropharyngeal cancer such as hypopharyngeal cancer, laryngeal cancer, nasopharyngeal cancer, and oropharyngeal cancer; stomach cancer such as lymphomas, gastric stromal tumors, and carcinoid tumors; testicular cancer such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin disease, non-Hodgkin lymphomas carcinoids or carcinoid tumors; rectal cancer; and colon cancer. In embodiments, a cancer includes a malignant thyroid disorder such as for example a follicular carcinoma, a follicular variant of papillary thyroid carcinomas, a medullary carcinoma, or a papillary carcinoma.

In embodiments, a disease or condition is not cancer. In embodiments, a disease or condition is acute. In other cases, a disease or condition is chronic. Suitable diseases and conditions may affect adults or pediatric subjects. A disease or condition may affect the brain, eyes, lungs, liver, bladder, kidneys, heart, stomach, intestines, or combinations thereof. Non-limiting examples of diseases and conditions include: autoimmune, allergy, asthma, celiac, Chrohn's, colitis, heart disease, liver disease, kidney disease, lupus, rheumatoid arthritis, scleroderma, polychondritis, macular degeneration, schizophrenia, ataxia, myotonic dystrophy, Alzheimer's, Huntington's, Kennedy's, fragile X syndrome, Autism, Inflammation, ALS, drug addiction, hemophilia, thalassemia, factor X deficiency, anemia, SCID, neuropathy, cystic fibrosis, cirrhosis, osteoporosis, atrophy, diabetes, stroke, hepatitis, epilepsy, COPD, meningitis, metabolic disease, and combinations thereof.

In embodiments, a subject may have been diagnosed with a disease or condition. In embodiments, the subject may have been diagnosed prior to treating with a peptide or pharmaceutical composition thereof provided herein. In embodiments, diagnosing a subject with a disease or condition includes diagnosing with a physical examination, a biopsy, a metabolite test, a radiological image, a blood test, a urine test, an antibody test, or any combination thereof. In embodiments, a radiological image includes a computed tomography (CT) image, a nuclear scan, an X-Ray image, a magnetic resonance image (MRI), an ultrasound image, or any combination thereof.

In embodiments, the method includes administering a second therapy. In embodiments, a second therapy includes an antibiotic, an antiviral, a cancer treatment, a neurological treatment, a steroid, an anti-inflammatory treatment, or any combination thereof. In embodiments, a second therapy includes surgery, chemotherapy, radiation therapy, immunotherapy, hormone therapy, a checkpoint inhibitor, targeted drug therapy, adoptive immunotherapy, anti-angiogenic agents, chimeric antigen receptor (CAR) T-cell therapy, gene editing therapy, a protein knockdown therapy, RNA editing therapy, or any combination thereof.

### Methods

Provided herein are, inter alia, platform-based screening methods that use overexpressed libraries of overlapping synthesized gene fragments that can be used to identify protein functional regions associated with an abnormal or disease cell fitness (e.g., cancer cell fitness), as well as dominant negative inhibitors of abnormal or disease cell growth (e.g., cancer cell growth) and as inhibitors of infectious agents. The methods provided herein are useful for screening and identifying peptides or fragments thereof which modulate activity of a target protein in a cell. Thus, in an aspect is provided a method for identifying a peptide or fragment thereof in a cell, wherein the peptide or fragment thereof modulates the activity of a target protein in the cell.

In embodiments, the screening methodology described herein includes providing a plurality of gene fragments which code for potentially inhibiting peptides for a targeted gene or genes (e.g., oncogenes, cell surface receptors, viral binding ligands etc.). In embodiments, the plurality of gene fragments may be generated by in silico pooled oligonucleotide synthesis technologies to generate large gene fragment libraries for use in the screening methods provided herein. Concepts from screening methodologies for CRISPR-based technologies can be used with the screening methodologies provided herein, including the CRISPR based screening methods found in Wang, T., Wei, J. J., Sabatini, D. M. & Lander, E. S., Science 343, 80-84 (2014); Gilbert, L. A. et al., Cell 159, 647-661 (2014); Shalem, O., Sanjana, N. E. & Zhang, F., Nat Rev Genet 16:299-311 (2015); Shalem, O. et al., Science 343, 84-87 (2014) each of which is hereby incorporated by reference in its entirety.

In embodiments, the gene fragments used in the screening methods provided herein are gene fragments of one or more oncogenes. Examples of oncogenes, include are but not limited to, MCL-1, BCR, BRAF, JAK1, JAK2, VEGF, EGFR, ALK, CDK1, CDK2, CDK3, CDK3, CDK4, BRCA, PIK3CA, MEK, C-KIT, NRAS, ABCB11, ANTXR2, BCOR, CDKN1B, CYP27A1, EMD, FANCF, ABCC8, APC, BCORL1, CDKN2A, CYP27B1, EP300, FANCG, ABCC9, AR, BLM, CEP290, DAXX, EPCAM, FANCI, ABCD1, ARID1A, BMPR1A, CFTR, DBT, EPHA5, FANCL, ABL1, ARID2, RAF1, CHEK1, DCC, EPHB2, FANCM, ACADM, ARSA, BRCA1, CHEK2, DCX, ERBB2, FAS, CADS, ASAHI, BRCA2, CHM, DDB2, ERBB3, FAT3, ACADVL, ASCC1, BRIP1, CIC, DDR2, ERBB4, FBX011, ACTC1, ASL, BTD, CLN3, DES, ERCC2, FBXO32, ACTN2, ASPA, BTK, CLN5, DHCR7, ERCC3, FBXW7, ACVRIB, ASS1, BUB1B, CLN6, DICER1, ERCC4, FGD4, ADA, ASXL1, CALR3, CLN8, DIS3L2, ERCC5, FGFR1, ADAMTS13, ATM, CARD11, COL1A2, DKC1, ERCC6, FGFR2, ADAMTS2, ATP4A, CASP8, COL4A3, DLD, ERRFI1, FGFR3, AGA, ATP6VOD2, CAV3, COL4A4, DMD, ESCO2, FH, AGL, ATP7A, CBFB, COL7A1, DNAJB2, ESR1, FKTN, AGPS, ATP7B, CBL, COX15, DNMT3A, ETV6, FLCN, AHI1, ATP8B1, CBLB, CREBBP, DSC2, EXOC2, FLT3, AIP, ATR, CBLC, CRLF2, DSE, EXT1, FMR1, AKAP9, ATRX, CBS, CRTAP, DSG2, EXT2, FUBP1, AKT1, AXIN1, CCDCl78, CRYAB, DSP, EYA4, FZD3, AKT2, AXIN2, CCNEI, CSFIR, DTNA, EZH2, G6PC, ALB, BAG3, CD79A, CSMD3, ECT2L, F11, GAA, ALDH3A2, BAI3, CD79B, CSRP3, EDA, F5, GABRA6, ALDOB, BAP1, CD96, CTNNB1, EDN3, FAH, GALNT12, ALK, BARD1, CDC27, CTNS, EDNRB, FAM46C, GALT, ALS2, BAX, CDC73, CTSK, EED, FANCA, GATA1, AMERI, BAZ2B, CDH1, CUBN, EGFR, FANCB, GATA2, AMPD1, BCKDHA, CDH23, CYLD, EGR2, FANCC, GATA3, AMPH, BCKDHB, CDK12, CYP11A1, EHBP1, FANCD2, GATADI, ANTXR1, BCL6, CDK4, CYP21A2, ELMO1, FANCE, GBA, GCDH, JAK1, MDM2, NEK2, PLOD1, ROS1, SMPD1, GJB2, JAK2, MECP2, NEXN, PLP1, RPGRIP1L, SOX10, GLA, JAK3, MED12, NF1, PMP22, RS1, SOX2, GLB1, JUP, MEFV, NF2, PMS2, RSPO1, SPEG, GLI1, KAT6A, MEN1, NFE2L2, POLD1, RTEL1, SPOP, GLI3, KCNQ1, MET, NFKBIA, POLE, RUNX1, SRC, GLMN, KDM4B, MFSD8, NIPA2, POLH, RUNX1T1, SSTR1, GNA11, KDM6A, MIER3, NKX3-1, POMGNT1, RYR2, STAG2, GNAQ, KDR, MITF, NOTCH1, POMT1, SIPR2, STAR, GNAS, KEAP1, MKS1, NOTCH2, POUIF1, SAMD9L, STK11, GNPTAB, KIF1B, MLH1, NPC1, POU6F2, SBDS, SUFU, GPC3, KIT, MLH3, NPC2, PPMIL, SCN11A, SUZ12, GPC6, KLF6, MMAB, NPHP1, PPP2R1A, SCN5A, SYNE3, GPR78, KLHDC8B, MPL, NPHP4, PPT1, SCNNIA, TAZ, GRIN2A, KMT2A, MPZ, NPM1, PRDMI, SCNNIB, TBX20, GRM8, KMT2C, MRE11A, PRKAG2, SCNN1G, TCAP, GXYLT1, KMT2D, MSH2, NRCAM, PRKARIA, SCO2, TCERG1, H3F3A, KRAS, MSH3, NTRK1, PRKDC, SDHA, TCF7L2, HADHA, KREMEN1, MSH6, NUP62, PROC, SDHAF2, TERT, HADHB, LICAM, MSMB, OR5L1, PROP1, SDHB, TET2, HBB, LAMA2, MSR1, OTC, PRPF40B, SDHC, TFG, HESX1, LAMA4, MTAP, OTOP1, PRX, SDHD, TGFB3, HEXA, LAMP2, MTHFR, PAH, PSAP, SEPT9, TGFBR1, HEXB, LDB3, MTM1, PALB2, PSEN1, SETBP1, TGFBR2, HFE, LEPRE1, MTOR, PALLD, PSEN2, SETD2, THSD7B, HGSNAT, LIG4, MUC16, PAX5, PTCH1, SF1, TINF2, HIST1H3B, LMNA, MUT, PAX6, PTCH2, SF3A1, TMC6, HNF1A, LPAR2, MUTYH, PBRMI, PTEN, SF3B1, TMC8, HRAS, LRP1B, MYBPC3, PCDH15, PTGFR, SGCD, TMEM127, HSPH1, LRPPRC, MYC, PCGF2, PTPN11, SGSH, TMEM43, IDH1, LRRK2, MYD88, PDE11A, PTPN12, SH2B3, TMEM67, IDH2, LYST, MYH6, PDGFRA, RAC1, SLC25A4, TMPO, IGF2R, MAP2K1, MYH7, PDHA1, RAD21, SLC26A2, TNFAIP3, IGHMBP2, MAP2K2, MYL2, PDZRN3, RAD50, SLC37A4, TNFRSF14, IGSF10, MAP2K4, MYL3, PEX1, RAD51B, SLC7A8, TNNC1, IKBKAP, MAP3K1, MYLK2, PEX7, RAD51C, SLC9A9, TNNI3, IKZF1, MAP4K3, MYO1B, PHF6, RAD51D, SLX4, TNNT1, IKZF4, MAP7, MYO7A, PIK3CA, RARB, SMAD2, TNNT2, IL2RG, MAPK10, MYOZ2, PIK3CG, RB1, SMAD4, TP53, IL6ST, MASIL, MYPN, PIK3R1, RBM20, SMARCA4, TPM1, IL7R, MAX, NBN, PKHD1, RECQL4, SMARCB1, TPP1, INVS, MC1R, NCOA2, PKP2, RET, SMC1A, TRAF5, IRAK4, MCCC2, NCOR1, PLEKHG5, RHBDF2, SMC3, TRIO, ITCH, MCOLN1, NDUFA13, PLN, RNASEL, SMO, TRPV4, TRRAP, U2AF1, USH1C, WAS, WWP1, ZIC3, TSC1, U2AF2, USHIG, WBSCR17, XPA, ZNF2, TSC2, UBA1, USP16, WEE1, XPC, ZNF226, TSHB, UBR3, USP25, WNK2, XRCC3, ZNF473, TSHR, UROD, VCL, WRN, ZBED4, ZNF595, TTN, UROS, VHL, WT1, ZFHX3, HER2, and ZRSR2.

In embodiments, the oncogene includes an oncogene selected from KRAS, HRAS, NRAS, RAF1, BRAF, ARAF, Myc, Max, FBXW7, and EGFR. In embodiments, the oncogene includes KRAS, HRAS, NRAS, RAF1, BRAF, ARAF, Myc, Max, FBXW7, or EGFR. In embodiments, the oncogene includes KRAS. In embodiments, the oncogene includes HRAS. In embodiments, the oncogene includes NRAS. In embodiments, the oncogene includes RAF1. In embodiments, the oncogene includes BRAF. In embodiments, the oncogene includes ARAF. In embodiments, the oncogene includes Myc. In embodiments, the oncogene includes Max. In embodiments, the oncogene includes FBXW7. In embodiments, the oncogene includes EGFR.

In embodiments, the gene fragments used in the screening methods provided herein are gene fragments of one or more receptors used as ligands for viruses. Examples of such receptor, include but are not limited to, CD4, poliovirus receptor, ICAM-1, Integrin (VLA-2), Coxsackievirus-adenovirus receptor (CAR), HAVcr-1, VACM-1, laminin receptor, angiotensin-converting enzyme 1 or 2 (ACE-1 or -2), MCP (CD46), SLAM, nectin-1, nectin-2, TNFR family, dipeptidyl peptidase-4 or -8 or -9, and CD21. In embodiments, the gene fragment includes a gene fragment of CD4, the poliovirus receptor, ICAM-1, Integrin (VLA-2), the Coxsackievirus-adenovirus receptor (CAR), HAVcr-1, VACM-1, the laminin receptor, angiotensin-converting enzyme 1 or 2 (ACE-1 or -2), MCP (CD46), SLAM, nectin-1, nectin-2, the TNFR family, dipeptidyl peptidase-4 or -8 or -9, or CD21. In embodiments, the gene fragment includes a gene fragment of CD4. In embodiments, the gene fragment includes a gene fragment of the poliovirus receptor. In embodiments, the gene fragment includes a gene fragment of ICAM-1. In embodiments, the gene fragment includes a gene fragment of Integrin (VLA-2). In embodiments, the gene fragment includes a gene fragment of the Coxsackievirus-adenovirus receptor (CAR). In embodiments, the gene fragment includes a gene fragment of HAVcr-1. In embodiments, the gene fragment includes a gene fragment of VACM-1. In embodiments, the gene fragment includes a gene fragment of the laminin receptor. In embodiments, the gene fragment includes a gene fragment of angiotensin-converting enzyme 1 or 2 (ACE-1 or -2). In embodiments, the gene fragment includes a gene fragment of angiotensin-converting enzyme 1 (ACE-1). In embodiments, the gene fragment includes a gene fragment of angiotensin-converting enzyme 2 (ACE-2). In embodiments, the gene fragment includes a gene fragment of MCP (CD46). In embodiments, the gene fragment includes a gene fragment of SLAM. In embodiments, the gene fragment includes a gene fragment of nectin-1. In embodiments, the gene fragment includes a gene fragment of nectin-2. In embodiments, the gene fragment includes a gene fragment of the TNFR family. In embodiments, the gene fragment includes a gene fragment of dipeptidyl peptidase-4 or -8 or -9. In embodiments, the gene fragment includes a gene fragment of dipeptidyl peptidase-4. In embodiments, the gene fragment includes a gene fragment of dipeptidyl peptidase-8. In embodiments, the gene fragment includes a gene fragment of dipeptidyl peptidase-9. In embodiments, the gene fragment includes a gene fragment of CD21.

The sequences of the oncogene/oncoproteins identified herein and above as well as the receptor identified herein have sequences that are known, as well as characterized biological activity. For example, AKT1 encodes RAC-alpha serine/threonine-protein kinase. This enzyme belongs to the AKT subfamily of serine/threonine kinases that contain SH2 (Src homology 2-like) domains. In embodiments, it is commonly referred to as PKB, or by both names as “Akt/PKB”. AKT1 was originally identified as the oncogene in the transforming retrovirus, AKT8. In embodiments, “BRAF” is a human gene that encodes a protein called B-Raf. The gene can also be referred to as proto-oncogene B-Raf and v-Raf murine sarcoma viral oncogene homolog B, while the protein can be more formally known as serine/threonine-protein kinase B-Raf. In embodiments, the B-Raf protein is involved in sending signals inside cells which are involved in directing cell growth. In embodiments, the mammalian target of rapamycin (mTOR), sometimes also referred to as the mechanistic target of rapamycin and FK506-binding protein 12-rapamycin-associated protein 1 (FRAP1), is a kinase that in humans is encoded by the MTOR gene. mTOR is a member of the phosphatidylinositol 3-kinase-related kinase family of protein kinases. mTOR links with other proteins and serves as a core component of two distinct protein complexes, mTOR complex 1 and mTOR complex 2, which regulate different cellular processes. In particular, as a core component of both complexes, mTOR functions as a serine/threonine protein kinase that regulates cell growth, cell proliferation, cell motility, cell survival, protein synthesis, autophagy, and transcription. mTOR also functions as a tyrosine protein kinase that promotes the activation of insulin receptors and insulin-like growth factor 1 receptors. Over-activation of mTOR signaling significantly contributes to the initiation and development of tumors and mTOR activity was found to be deregulated in many types of cancer including breast, prostate, lung, melanoma, bladder, brain, and renal carcinomas.

The methods provided herein including embodiments thereof can be used to profile MHC binding peptides; disrupt pathogenic protein aggregation relevant to tauopathies, amyloid plaques etc.; engineer or profile cytokine/receptor relevant to cancer immunotherapy as well as autoimmune disorders; identify peptides mediating regenerative phenotypes such as angiogenesis; discover anti-aging therapeutics; and/or discovering pain modulating peptides.

Provided herein are, inter alia, methods which can be utilized for screening. Thus, in an aspect is provided, a method for screening genes or proteins encoded by genes for inhibitory or stimulatory activity in a cell. In embodiments, a method includes expressing at least a fragment of a gene in a target cell. In embodiments, a method of screening for at least partially reducing or at least partially increasing the activity of a target protein, a protein that may interact downstream with a target protein, or both includes expressing one or more fragments of a gene in a target cell. In embodiments, a gene fragment can be expressed from a vector, such as a polynucleotide (e.g., a plasmid) or a viral vector including a nucleic acid encoding the gene fragment. In embodiments, a gene fragment includes at least a portion of a target protein. In embodiments, a gene fragment can be from: about 20 nucleotides to about 1000 nucleotides, about 20 nucleotides to about 100 nucleotides, about 100 nucleotides to about 500 nucleotides, about 60 nucleotides to about 150 nucleotides, or about 500 nucleotides to about 1000 nucleotides in length. In embodiments, a method of at least partially reducing the activity of a target protein or at least partially reducing the activity of a protein that interacts downstream with a target protein includes measuring the at least partial reduction of activity by determining a change in gene expression of a treated cell relative to the level of a gene expression in an untreated cell in an in vitro assay. In embodiments, a method of screening for at least partially reducing or at least partially increasing the activity of a target protein, a protein that may interact downstream with a target protein, or both includes measuring the at least partial reduction of activity by determining a change in gene expression of a treated cell relative to the level of a gene expression in an untreated cell in an in vitro assay. In embodiments, a change in gene expression can be determined by quantitative reverse transcriptase polymerase chain reaction, RNA-seq or both. In embodiments, a fragment of a gene can encode for a polypeptide including a sequence having at least about: 70%, 75%, 80%, 85%, 90%, 95%, 98%, or 99% sequence homology to any one of the peptides having a sequence set forth in any one of SEQ ID NOs: 1-2027. In embodiments, a vector or plasmid can be transfected, electroporated, or transduced into the target cell. In embodiments, at least a portion of the target protein includes about: 5 amino acids to about 80 amino acids, 5 amino acids to about 20 amino acids, 10 amino acids to about 40 amino acids, 20 amino acids to about 60 amino acids, 20 amino acids to about 50 amino acids, or about 40 amino acids to about 80 amino acids. In embodiments, reduction of activity includes reduced cell growth.

Also provided herein are methods of making a pharmaceutical composition described herein. In embodiments, the methods include contacting a peptide or salt thereof with a pharmaceutically acceptable excipient, diluent or carrier.

In embodiments, a pharmaceutical composition described herein can be administered in a therapeutically effective amount to a subject (e.g., a human) in need thereof to at least partially prevent or treat a disease or condition. In embodiments, treating includes at least partially reducing or ameliorating at least one symptom of the disease or condition, such as reducing a growth of a tumor. The terms “treating,” “treatment,” and the like can be used herein to mean obtaining a desired pharmacologic effect, physiologic effect, or any combination thereof. In embodiments, a treatment can reverse an adverse effect attributable to the disease or disorder. In embodiments, the treatment can stabilize the disease or disorder. In embodiments, the treatment can delay progression of the disease or disorder. In embodiments, the treatment can cause regression of the disease or disorder. In embodiments, the treatment can prevent the occurrence of the disease or disorder. In embodiments, a treatment's effect can be measured. In embodiments, measurements can be compared before and after administration of the composition. For example, a subject can have medical images prior to treatment compared to images after treatment to show cancer regression. In embodiments, a subject can have an improved blood test result after treatment compared to a blood test before treatment. In embodiments, measurements can be compared to a standard.

### EXAMPLES

### Example 1

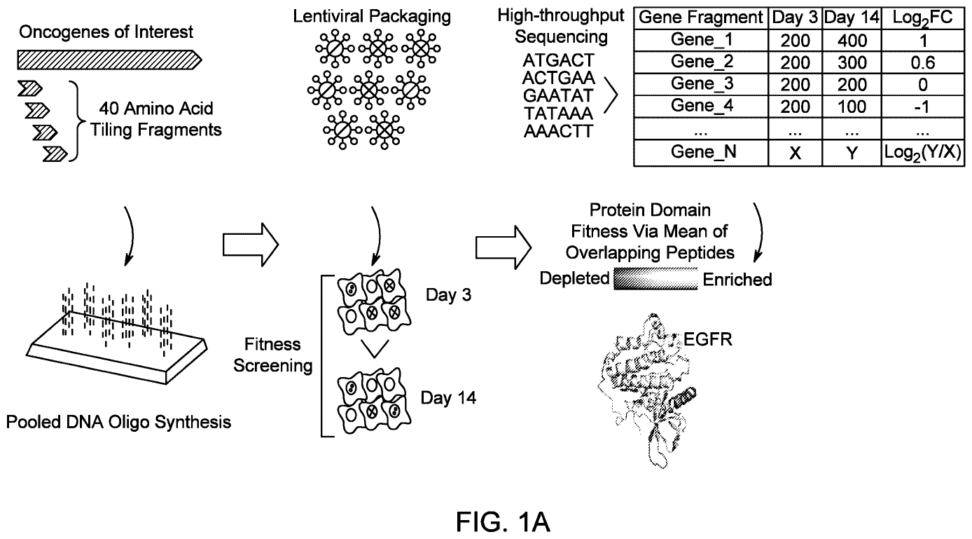

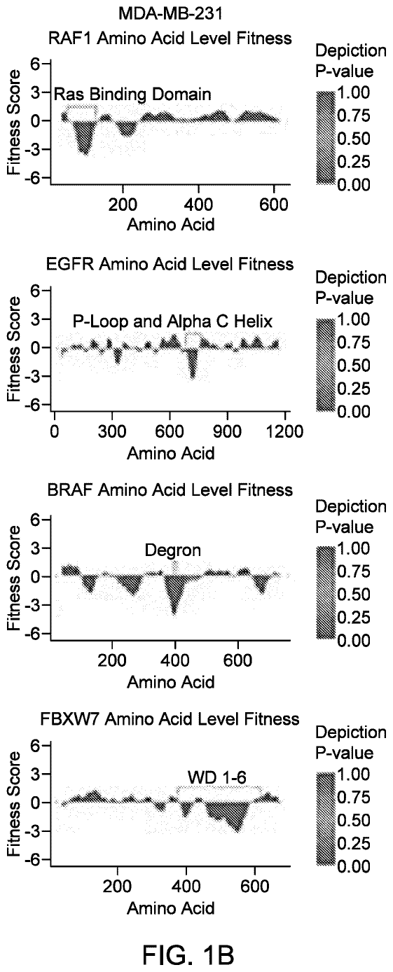

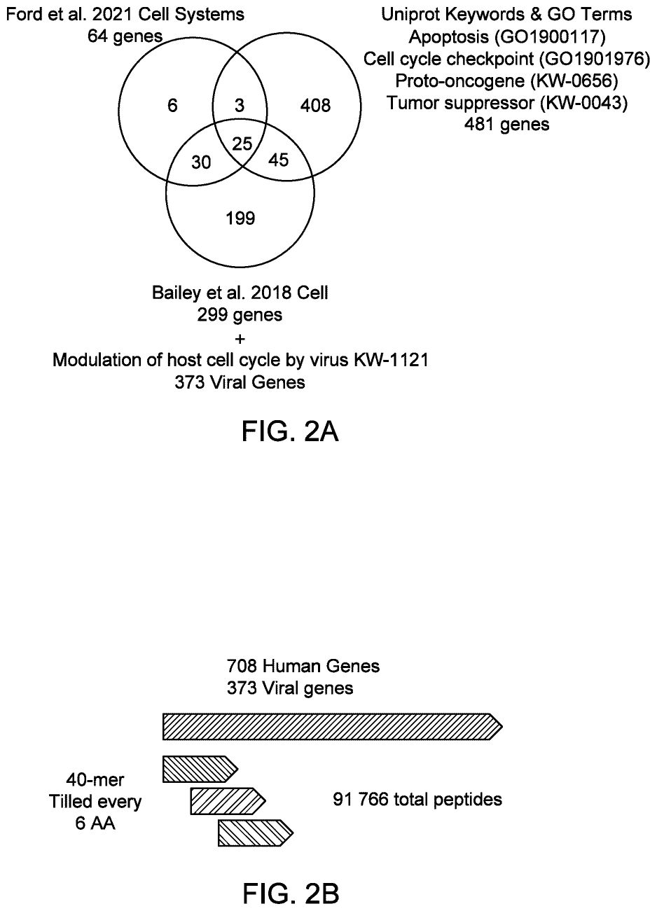

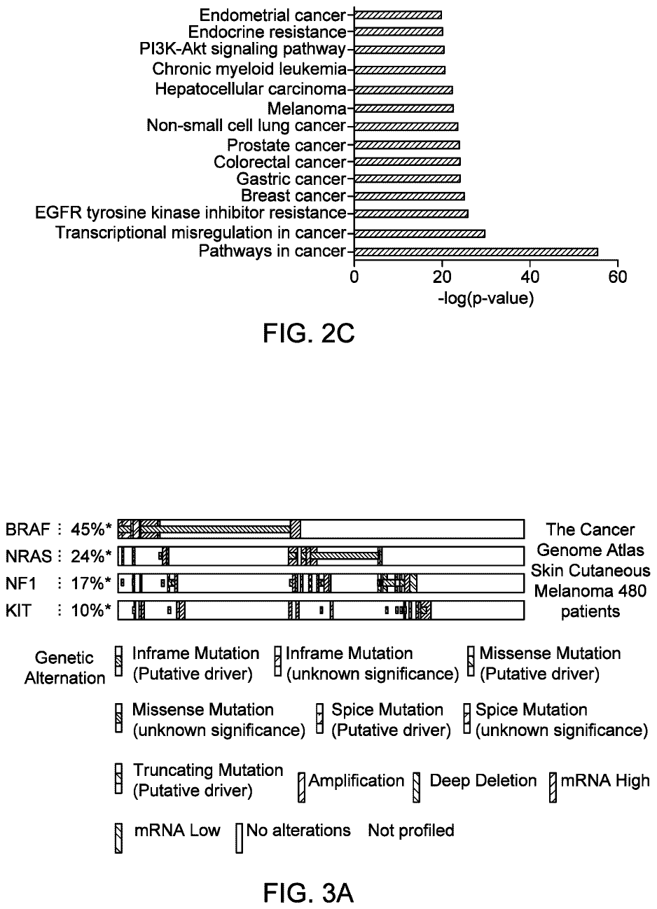

A peptide tilling screen was performed in A375 similarly to a screen done in MDA-MB-231 (FIG. 1) but with a different lentiviral library. The library (Pan-Cancer library) is composed of 1020 genes tiled every 6 amino acids into 40-mer amino acid long peptides. The gene selection was based on their implication in oncogenesis either being as oncogenes or tumor suppressors. The list was built using recent literature that identified new cancer drivers as well as databases such as Uniprot keywords (KW-0656: Proto-oncogene, KW-0043: Tumor suppressor), and Gene Ontology terms (GO1900117: Apoptosis, GO1901979: Cell cycle checkpoint) (FIG. 2). Aside from Human genes, a list of viral genes was also included (KW-1121: Modulation of host cell cycle by virus) that play roles in high jacking cell cycle progression and thus in oncogenesis (FIG. 2).

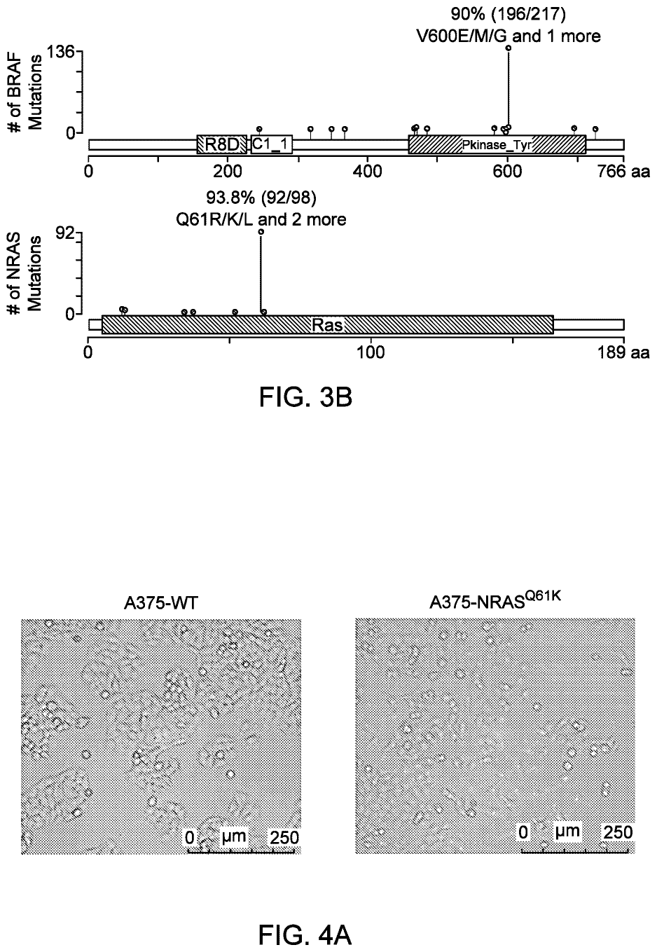

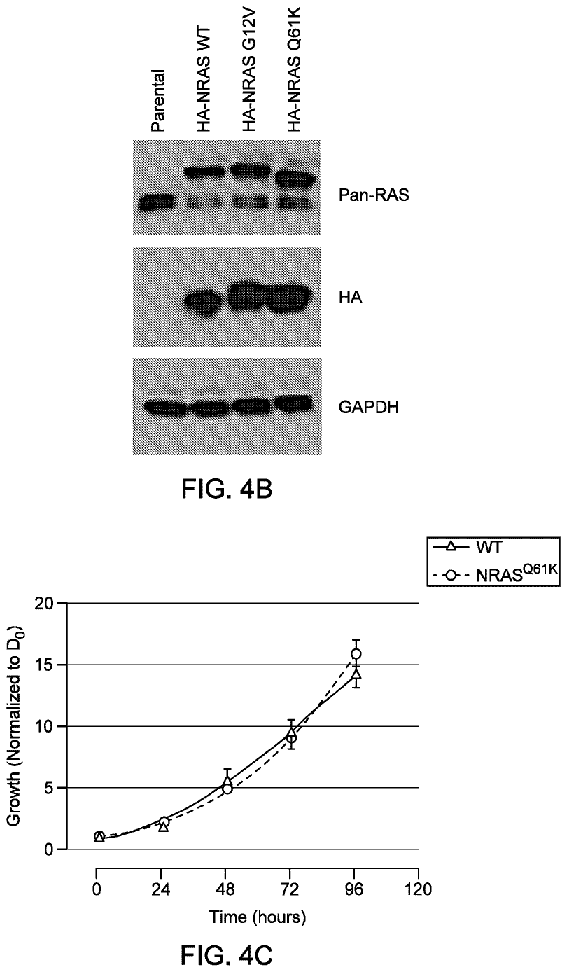

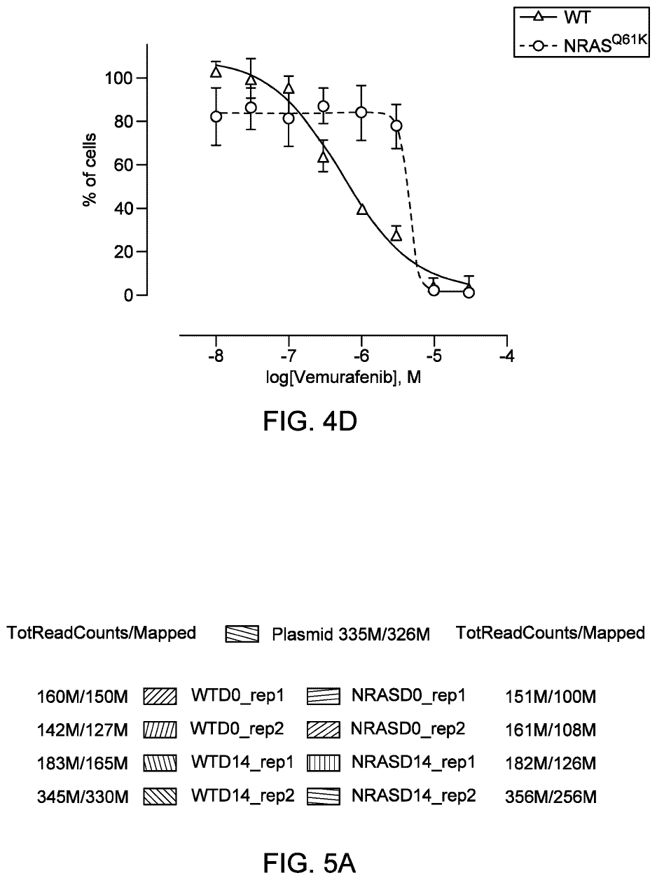

The model of study is melanoma, and this disease is stratified into 4 molecular subtypes, BRAF mutants, NRAS mutants, KIT amplification, NF1 mutations (FIG. 3A). The most prominent mutations found in patient affected with metastatic melanomas are BRAF^{V600E} and NRAS^{Q61K} (FIG. 3B). RAS play a key role in oncogenesis and resistance to treatments. Indeed, RAS activating mutations is found in ~20% of all human cancers and secondary NRAS mutation leads to acquired resistance to BRAF targeted therapy such as vemurafenib. Thus, the screen was performed in an A375 cell line model which derived from human BRAF^{V600E} melanoma as well as an engineered A375 transduced with HA-NRAS^{Q61K} (FIG. 4A, B). Both cell lines grow at a similar rate but the A375-NRAS^{Q61K} display more than 10-fold resistance to vemurafenib treatment compared to parental A375 (FIG. 4C, D).

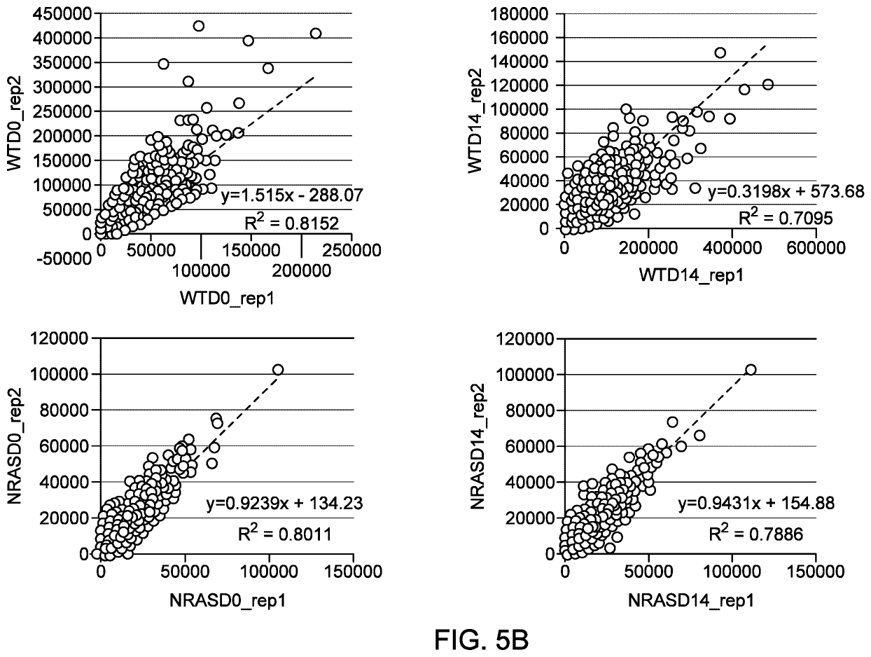

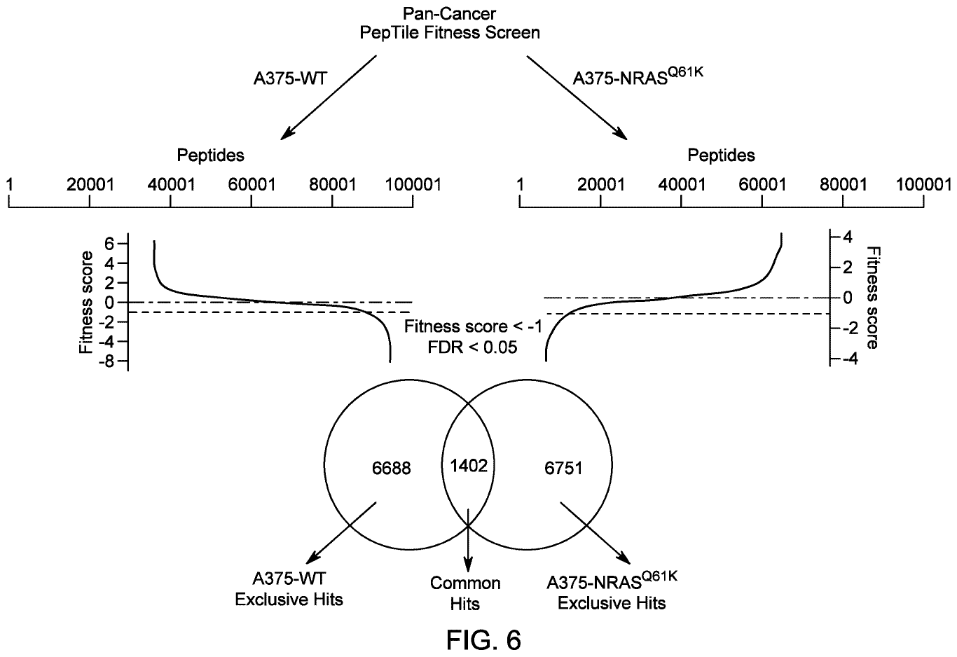

These cells were transduced with the Pan-Cancer library at a low multiplicity of infection (MOI=0.1-0.3). Then, genomic DNA was extracted at day 0 and day 14 post-transduction to quantify the abundance of peptide for each temporal conditions using Next-generation sequencing. Each experimental replicates were deep sequenced with at least 150 million reads and show a coverage between 82-90% of the initial Pan-Cancer Peptide library (FIG. 5). Then, for each individual peptides a cellular fitness score was calculated based on their enrichment between day 0 and day 14. 8090 peptides negatively affecting growth of A375-WT were identified and 8153 peptides for the A375-NRAS^{Q61K} cell line (FIG. 6). Most of those peptides are exclusive to their respective condition, however 1402 peptides were found to be common to both WT and NRAS^{Q61K}. Those common hits are found to be tiles from ~100 genes of the Pan-Cancer gene list and show a substantial biological function clustering.

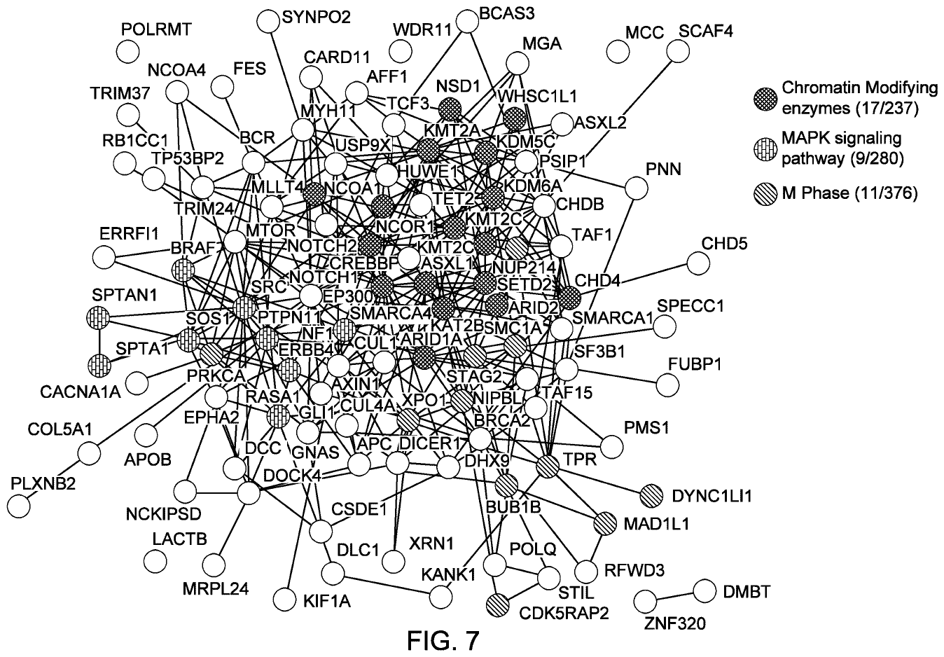

Peptides were also identified from genes involved in Chromatin Modifications, MAPK pathway, and Mitosis (FIG. 7). Interestingly those pathways are known to be therapeutics targets for melanoma treatment.

Out of the hits defined in the FIG. 7, a curated list of interesting peptides hits that are exclusive to A375-WT, exclusive to A375-NRAS^{Q61K} and common for both cell lines (Table 1) as well as an extended list of PepTile hits common to WT and NRAS 293 hits, WT exclusive 434 hits, NRAS exclusive 467 hits.

TABLE 1
Summary table of manually curated significant peptides hits from Pan-
Cancer fitness screen performed in A375-WT and A375-NRAS(Q61K).

| | | | A375- | | |
|---|---|---|---|---|---|
| | | A375-WT | NRAS | | Known |
| Peptide Tile | AA start | Fitness | Fitness | Protein | Biochemical |
| ID | position | Score | Score | domain | Function |
| MAD1L1_78 | 462 | −4.42 | −3.52 | Coiled Coil | Oligomerization |
| FZD3_55 | 324 | −3.21 | −3.27 | Transmembrane | Membrane Localization |
| ELF4_39 | 228 | −2.44 | −1.35 | ETS | DNA binding |
| BRAF_64 | 378 | −2.28 | −2.72 | CR2 | 14-3-3 binding |
| FER_38 | 222 | −2.21 | −1.48 | F-BAR | Dimerization |
| FLCN_43 | 252 | −1.76 | −1.99 | Longin | Interaction with FNIP2 |
| SMARCA4_257 | 1536 | −4.98 | −0.54 | Bromodomain | DNA binding |
| KIT_84 | 498 | −3.23 | −0.77 | Transmembrane | Membrane Localization |
| SOS1_21 | 120 | −2.56 | −0.81 | HF | Plasma membrane |
| | | | | | interaction |
| MYC_65 | 384 | −2.50 | −0.47 | bHLH | DNA binding |
| ARID2_13 | 72 | −2.41 | 0.85 | ARID | DNA binding |
| RAF1_14 | 78 | −1.79 | −0.50 | RBD | Interaction with RAS |
| MTOR_42 | 246 | −3.36 | 1.01 | HEAT | Interaction with |
| | | | | | RICTOR/RAPTOR |
| PIK3CA_38 | 222 | −2.72 | −0.85 | RBD | Interaction with RAS |
| SOX17_17 | 96 | −4.49 | −0.77 | HMG box | DNA binding |
| EP300_99 | 588 | −3.20 | 0.86 | KIX | Dimerization |
| SUZ12_23 | 132 | −2.12 | −0.27 | WDB1 | Interaction with Rbbp4 |
| SMC3_124 | 738 | −3.08 | 0.13 | Coiled Coil | Dimerization |
| CTNNB1_30 | 174 | −3.29 | −0.85 | Armadillo | Interaction with |
| | | | | | partners |
| AXIN1_22 | 126 | −2.88 | −0.54 | RGS | Interaction with APC |
| CDKN2B_4 | 18 | 0.36 | −3.63 | ANK | interaction with |
| | | | | | CDK4/6 |
| DOCK4_114 | 678 | 0.87 | −3.45 | Armadillo | Rho interaction |

TABLE 2
Exemplary auxiliary moieties that can be conjugated to the
peptides provided herein including embodiments thereof.
Sequence (N′ to C′) (PTD = protein
transduction domain)

PEG- (PTD)
GG- (PTD) -PEG- (PTD)
PEG- (PTD) -PEG- (PTD)
GG- (PTD) -PEG-PEG-PEG- (PTD)
PEG- (PTD) -PEG-PEG-PEG- (PTD)
GG- (PTD) -PEG- (PTD) -PEG- (PTD)
GG- (PTD) -PEG-PEG-PEG- (PTD) -PEG-PEG-PEG- (PTD)

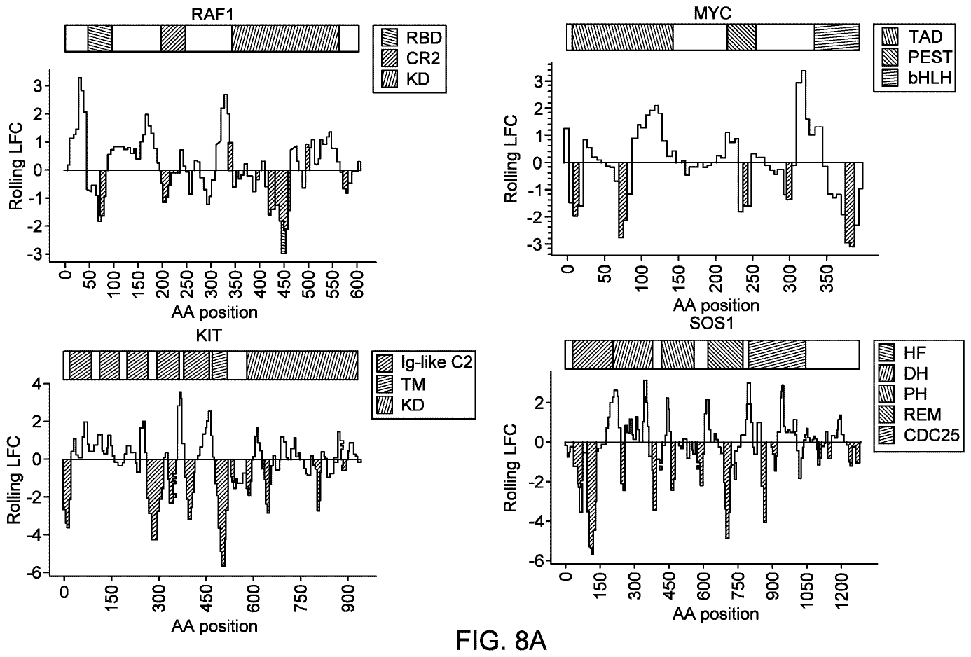

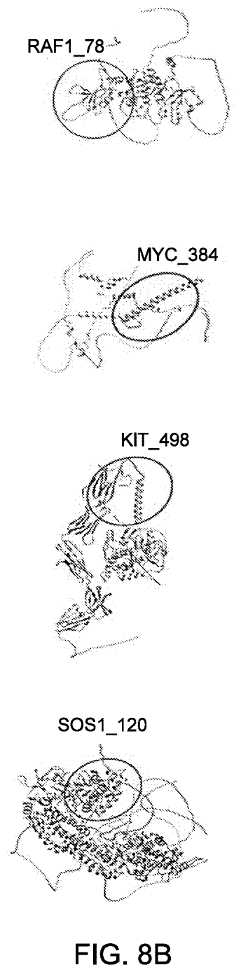

All those selected hits mapped to a known protein domain with known roles in protein-protein interaction or protein-DNA interaction. For the first round of validation assay, A375-WT exclusive hits list was used; MYC_65, RAF1_14 were picked, for which those peptides have already been identified and validated in a previous screen in MDA-MB-231, as well as newly identified hits such as KIT_84, and SOS1_21. When the fold change of every tile on the protein amino acid positions was mapped, functional regions of the protein affecting cell growth were identified. For example, on RAF1, peaks corresponding to the Ras-binding domain (RBD) were identified, and the kinase domain (FIG. 8A). Interestingly, characterization and work on RAF1_14 tile provided for a developed and stapled peptide that reduces tumor growth in vivo. In addition, a region of MYC that affect cell growth was identified and mapped on the DNA binding domain bHLH (FIG. 8). Interestingly this region of MYC is known to induce a dominant negative effect on cancer cell growth and have led to the development of Omomyc, which is currently in phase1/2 clinical trials. Omomyc is 92 amino acid long, and in the screen, significant 40 amino acid tiles were identified in that exact same region, suggesting potential size optimization for Omomyc (FIG. 8). To validate the results from the screen, A375-WT and -NRAS^{Q61K} were transduced with GFP_147, or RAF1_78, MYC_384, or KIT_499, or SOS_120 tiles and cell growth measured using CCK8 assay

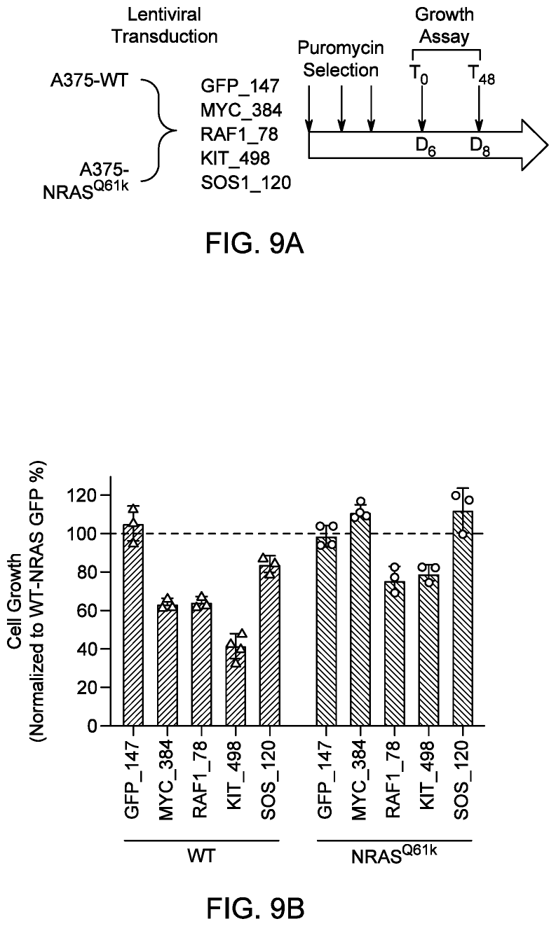

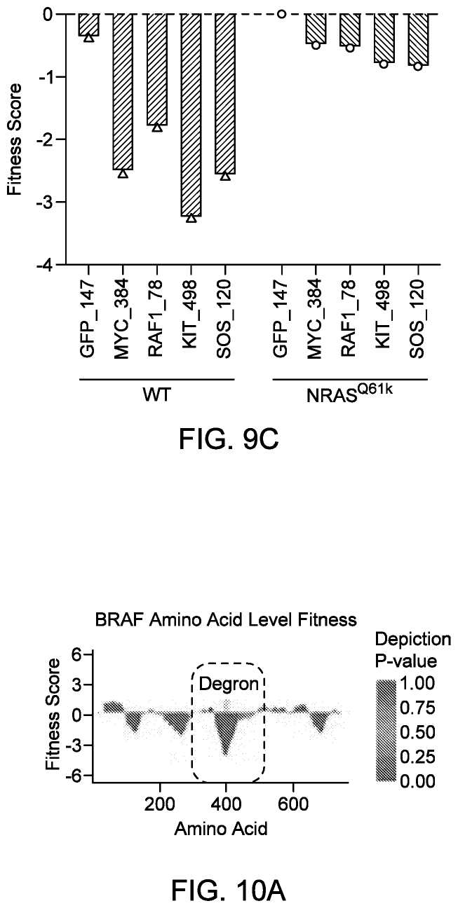

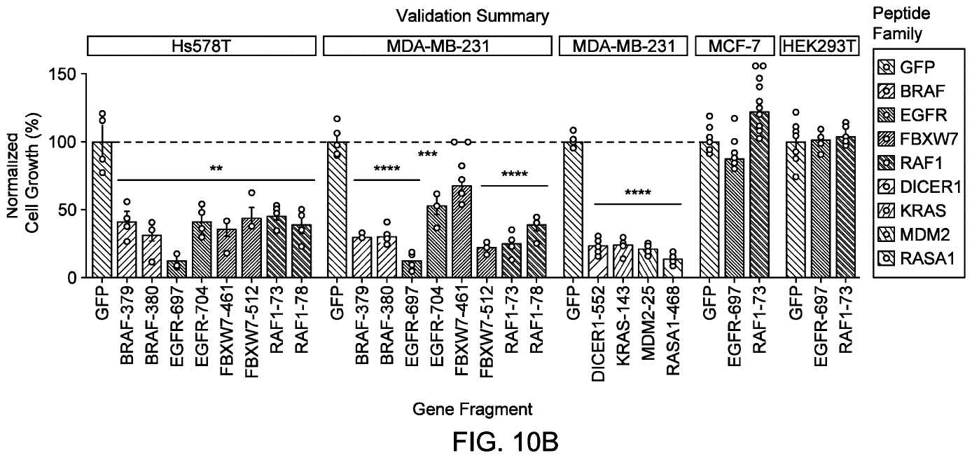

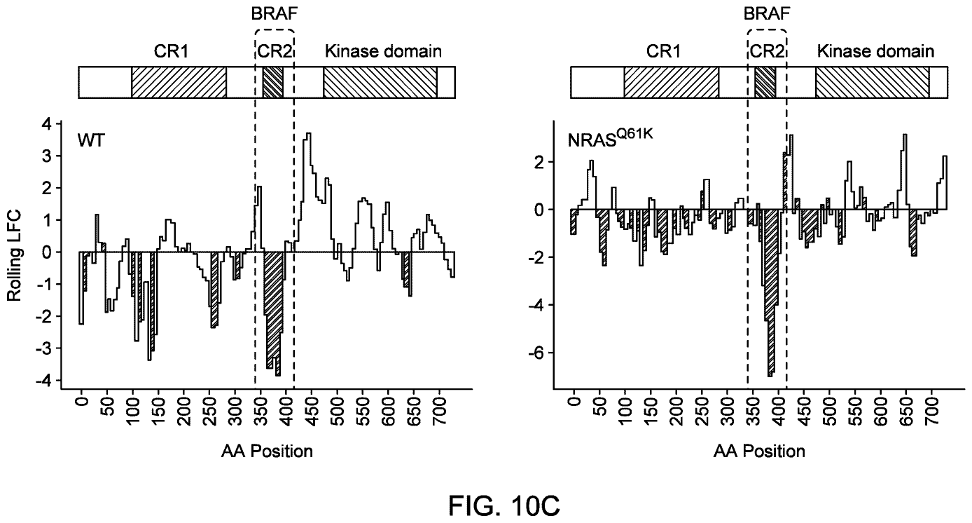

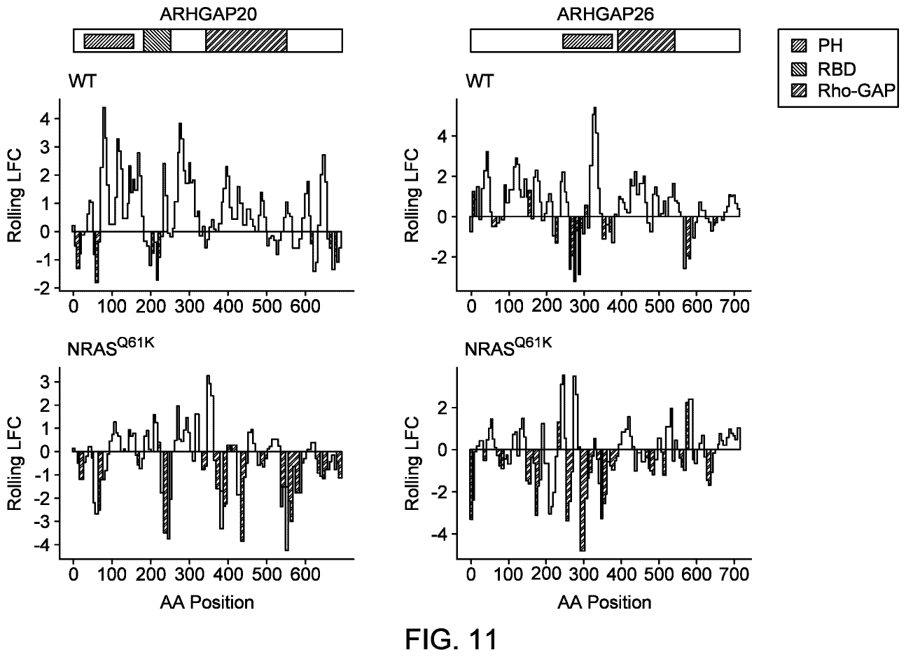

48 hours after plating (FIG. 9A). In A375-WT overexpression of MYC_384, RAF1_78, KIT_499 and SOS_120 decreases cell growth compared to GFP_147 overexpressing cells, whereas NRAS^{Q61K} growth is not affected by MYC_384, or SOS_120 and much less affected by RAF1_78, and KIT_499 (FIG. 9B). This result is consistent with the screening data for which a size effect difference was observed between A375-WT and -NRAS^{Q61K} for these selected peptides suggesting a good accuracy for this Pan-Cancer PepTile Screen dataset (FIG. 9C). Amongst the promising hits common to A375-WT and A375-NRAS^{Q61K}, a region of BRAF that has been previously identified and validated in MDA-MB-231 and Hs578T was identified (FIG. 10A, B), which correspond to the CR2 domain (FIG. 10C). The CR2 domain of BRAF contain a 14-3-3 binding site and play a role in BRAF-MEK autoinhibitory complex which regulates MAPK pathway activation. One of the goals of this contextual PepTile screen was to identify peptides that would impair growth of NRAS cell line but not the parental, to address overall NRAS-driven drug resistance. For instance, more than 6000 peptide hits were detected that are exclusive to A375-NRAS^{Q61K} (FIG. 6). For example, ARHGAP20 and ARHGAP26 show a very different PepTile profiles between A375-NRAS^{Q61K} and A375-WT (FIG. 11) and seem to have more of an effect in the A375-NRAS^{Q61K} condition. Altogether, the Pan-Cancer PepTile screen show the potential of finding new potential peptides to target NRAS melanoma, as well as mapping protein domains involved in cellular growth in a given context.

Adenosine-to-inosine (A-to-I) RNA editing is a post-transcriptional modification process that involves the conversion of adenosine to inosine in RNA molecules. This process is catalyzed by the adenosine deaminase acting on RNA (ADAR) family of enzymes and can result in changes to the RNA sequence, structure, and function. Recent studies have shown that A-to-I RNA editing plays a crucial role in cancer progression and tumorigenesis. For example, ADAR-mediated editing of microRNA (miRNA) binding sites in messenger RNAs (mRNAs) can lead to altered gene expression and contribute to cancer development. Targeting A-to-I RNA editing has emerged as a potential strategy for cancer therapy.

Additionally, A-to-I RNA editing has been shown to affect the stability and activity of various RNA molecules, including long non-coding RNAs (lncRNAs) and circular RNAs (circRNAs), which can promote or inhibit cancer growth. Several studies have demonstrated that inhibition of ADAR enzymes can reduce tumor growth and sensitize cancer cells to chemotherapy and immunotherapy. For example, two recent studies have showed that ADAR1 inhibition enhanced the efficacy of anti-PD-1 therapy in a mouse model of melanoma and lung adenocarcinoma. A-to-I RNA editing can influence the response of cancer cells to immunotherapy. For example, A-to-I editing of IFNAR1, the receptor for type I IFNs, can impair the IFN signaling pathway and promote cancer cell survival. A study by Karki et al. found that ADAR1 promote tumorigenesis via innate immune response and interferon pathway. Given the role of A-to-I RNA editing in the Interferon pathway and immune checkpoint regulation, targeting this process has emerged as a potential strategy for cancer immunotherapy. Inhibition of ADAR enzymes have been shown to enhance the response of cancer cells to IFN therapy and immune checkpoint blockade. For example, a study by Zhang et al. found that ADAR1 inhibition sensitized melanoma cells to IFN therapy and increased T cell infiltration in tumors. Thus, Peptide that perturb A-to-I editing has the potential to enhance immunotherapy in melanoma.

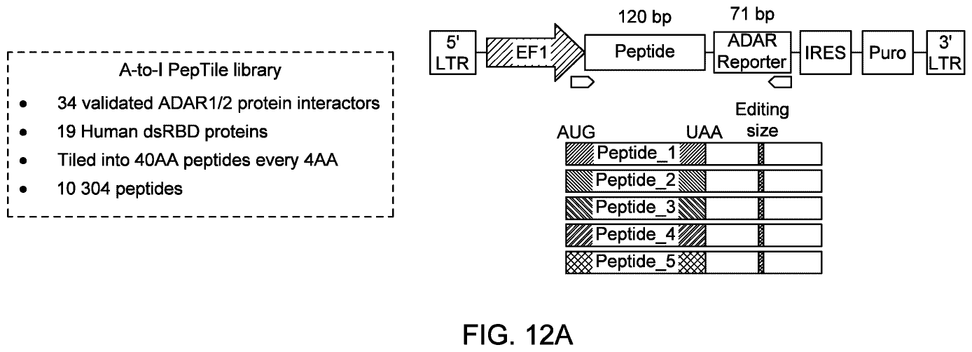

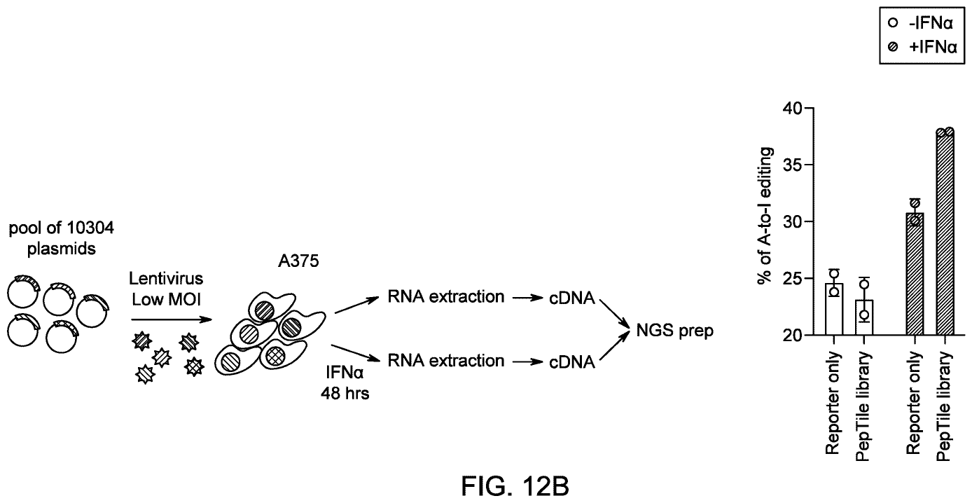

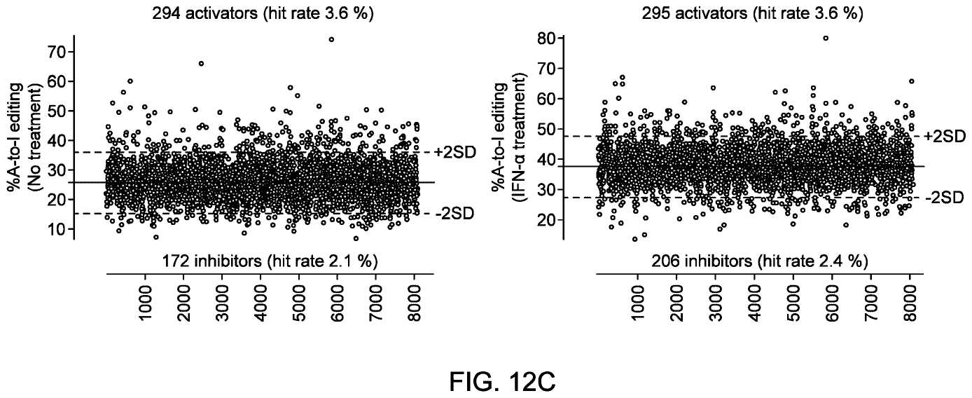

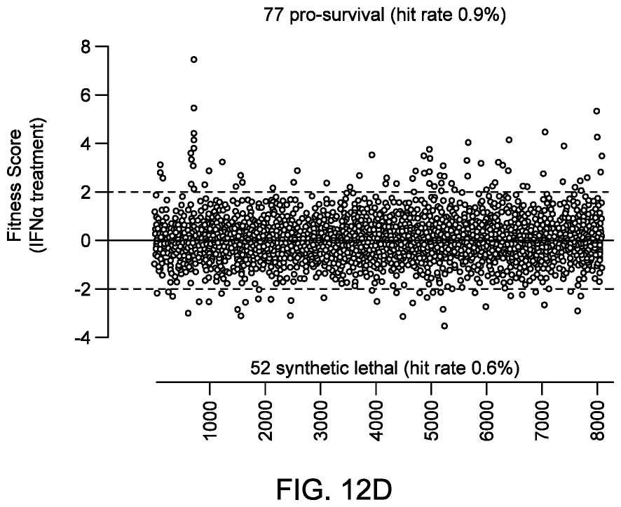

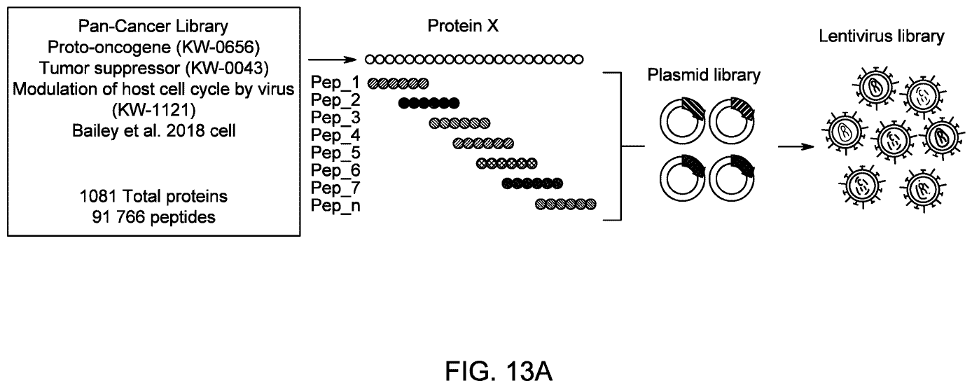

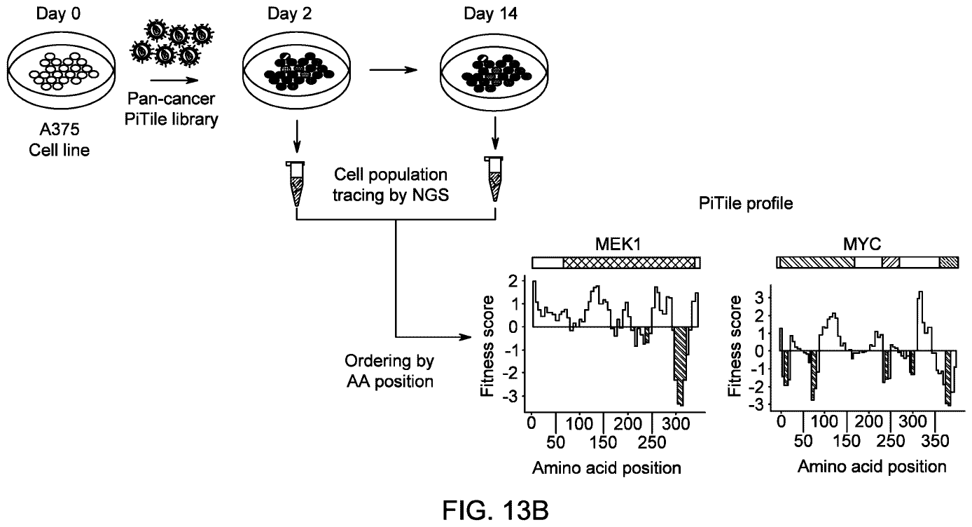

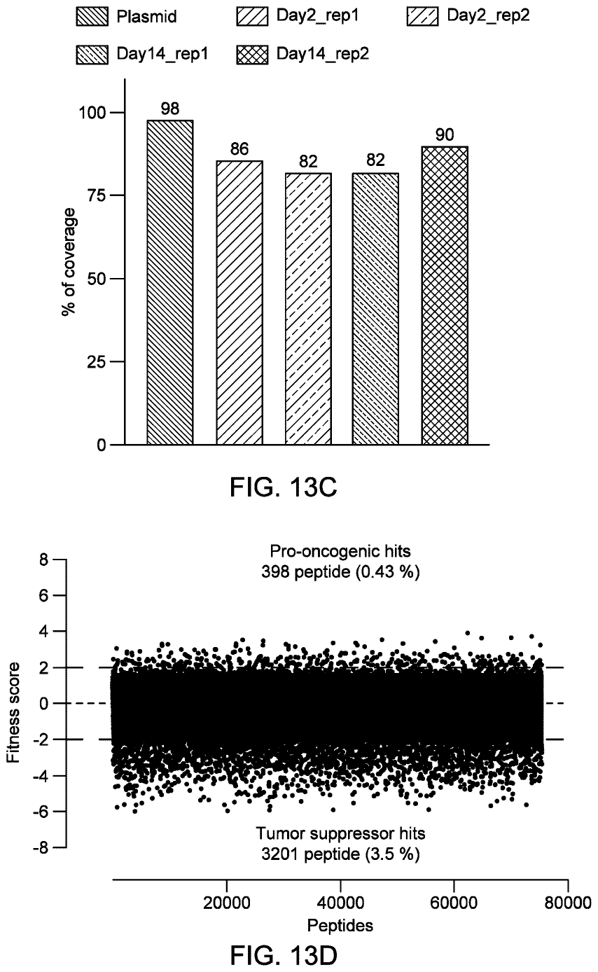

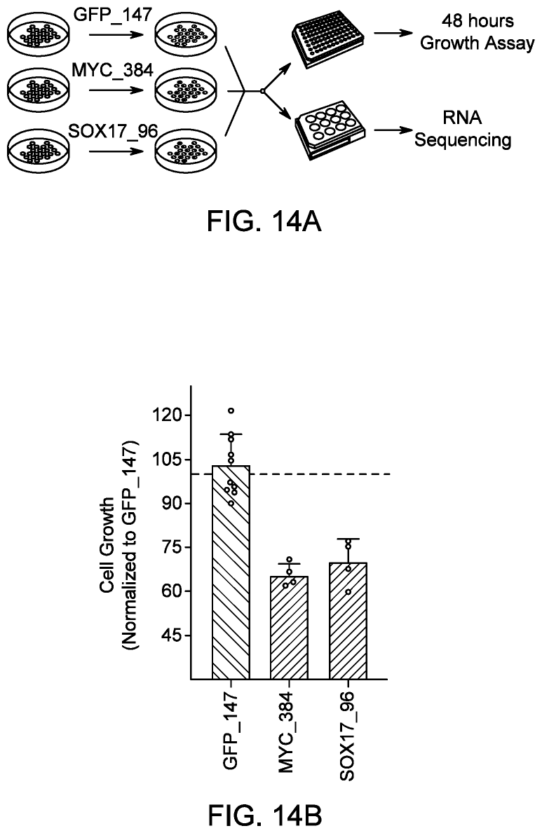

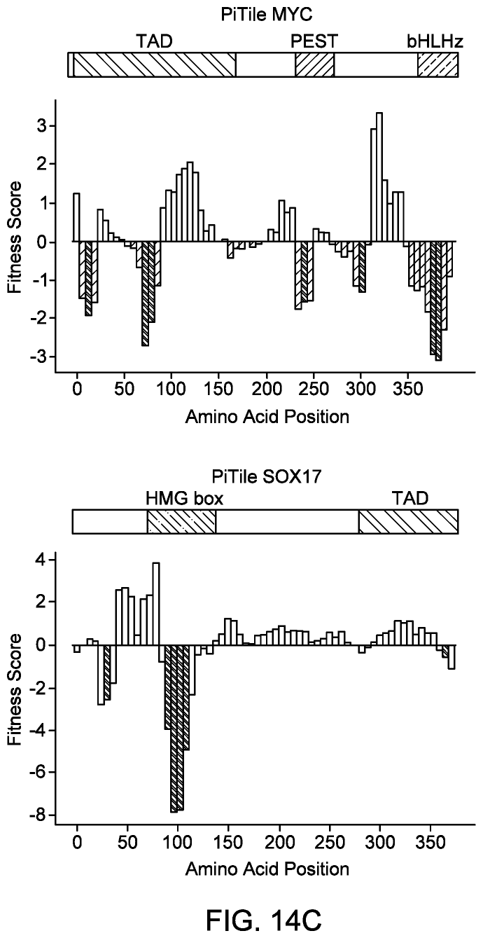

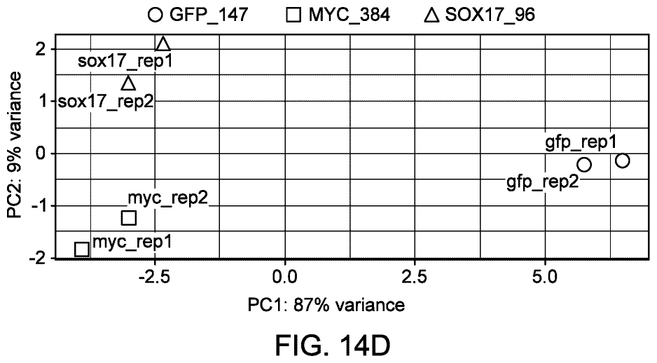

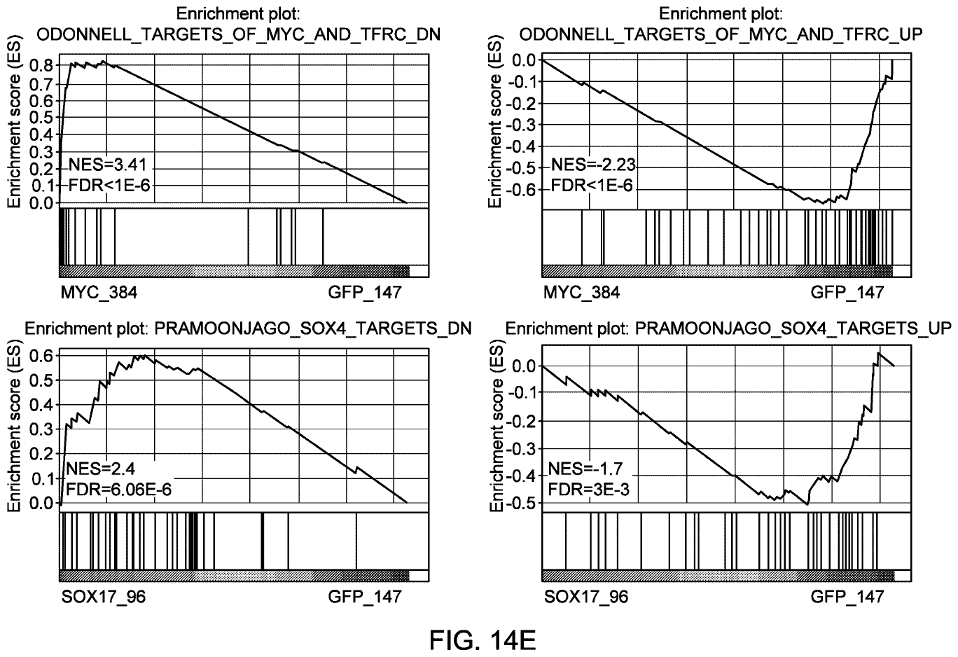

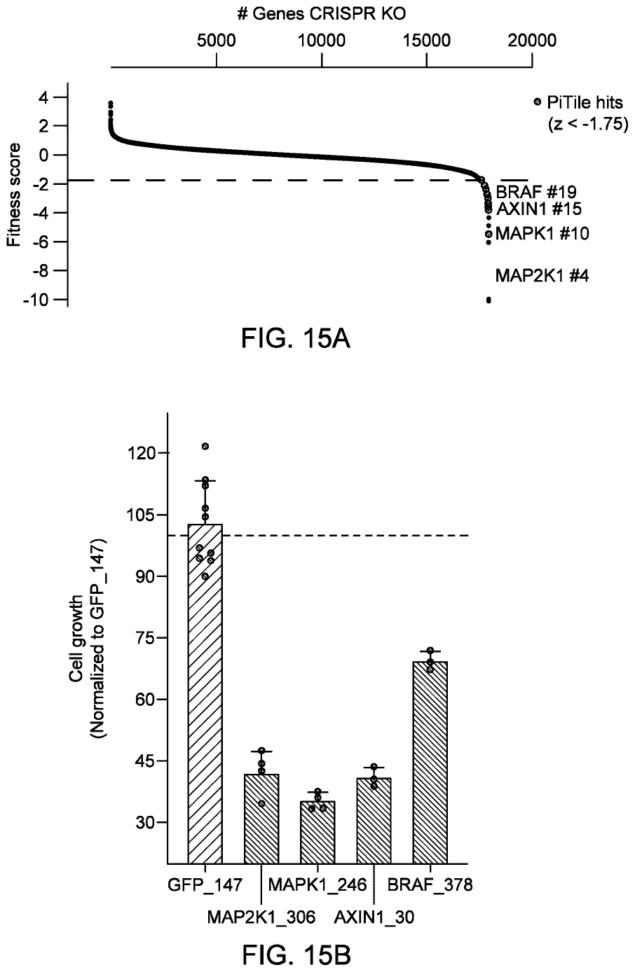

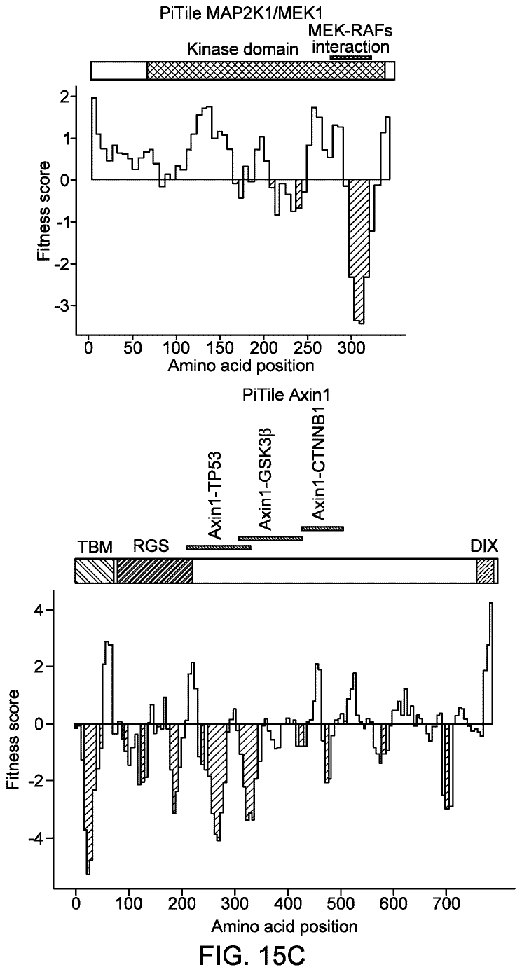

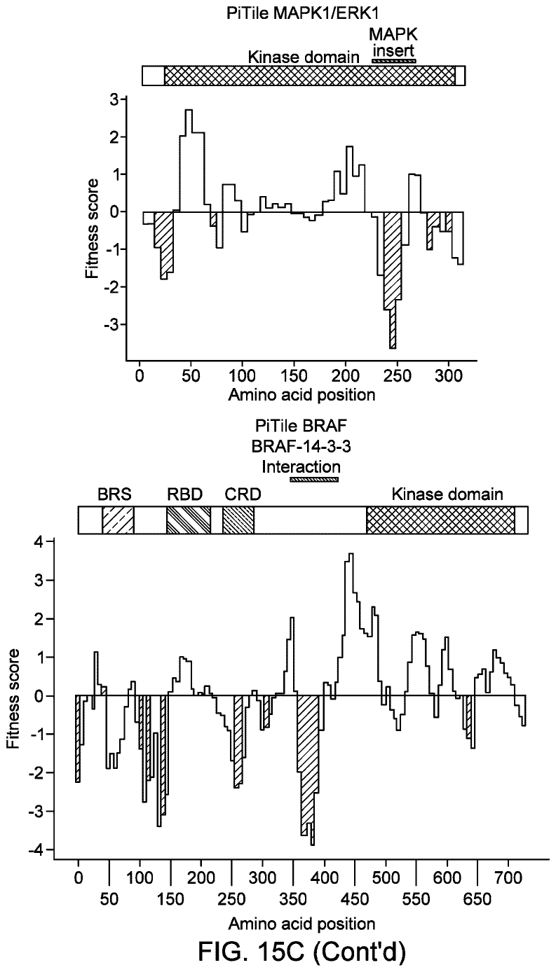

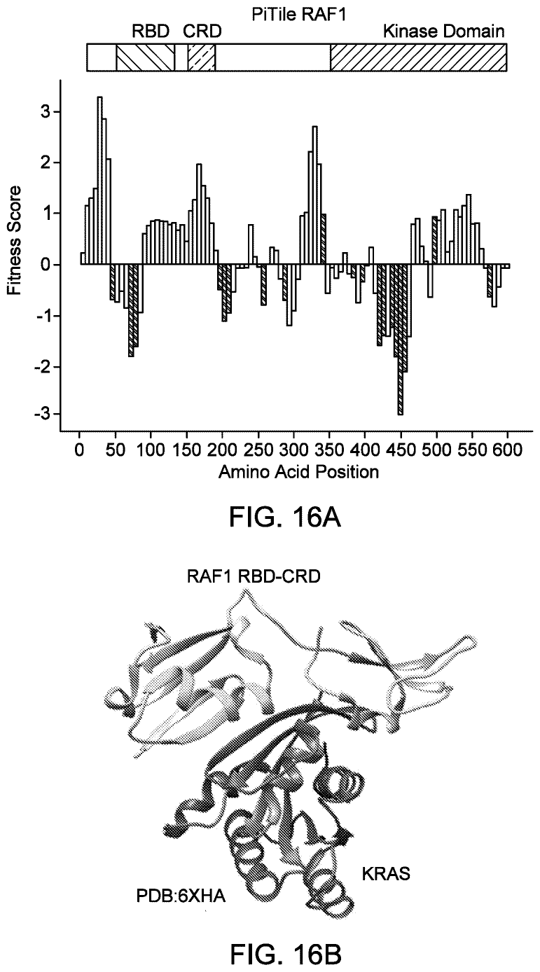

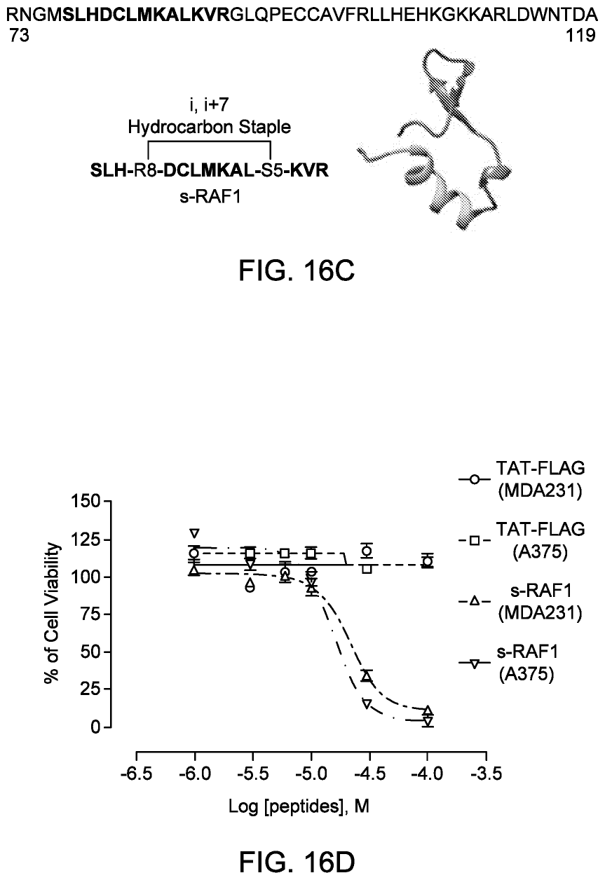

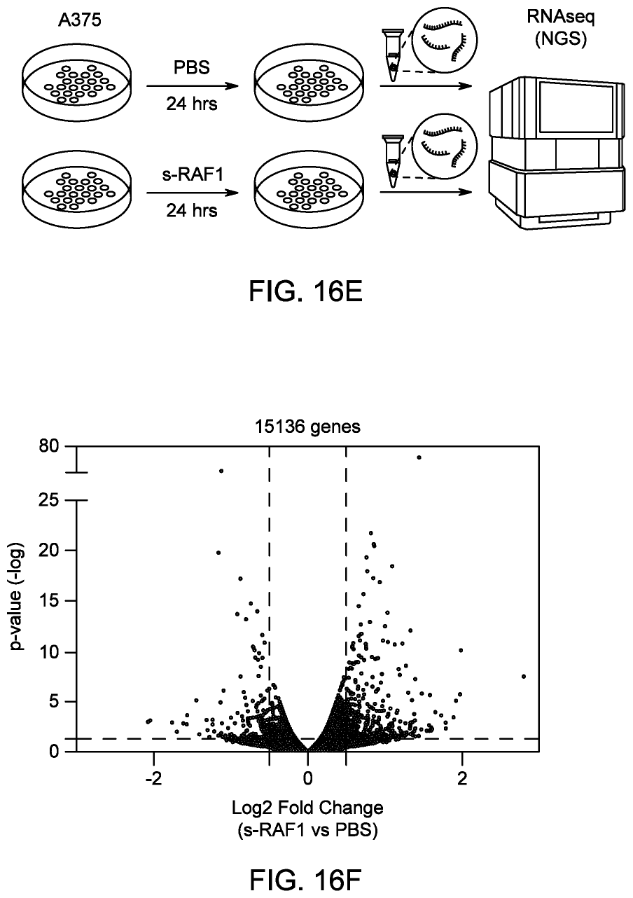

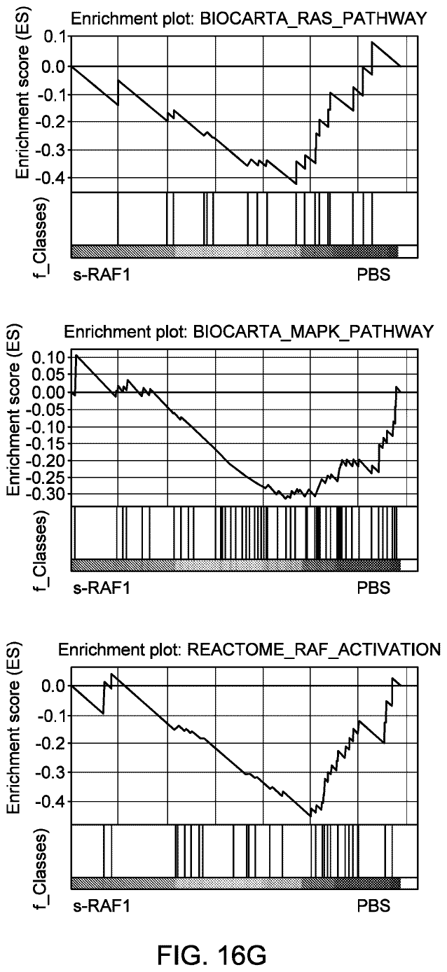

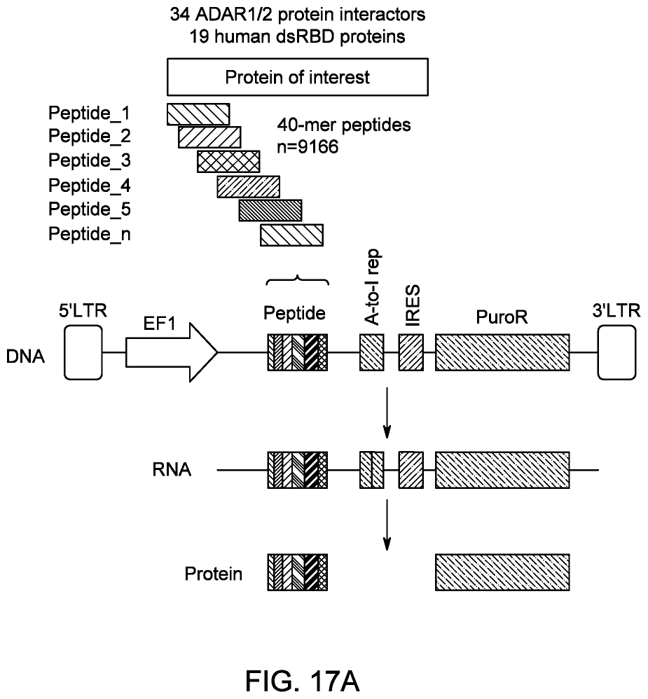

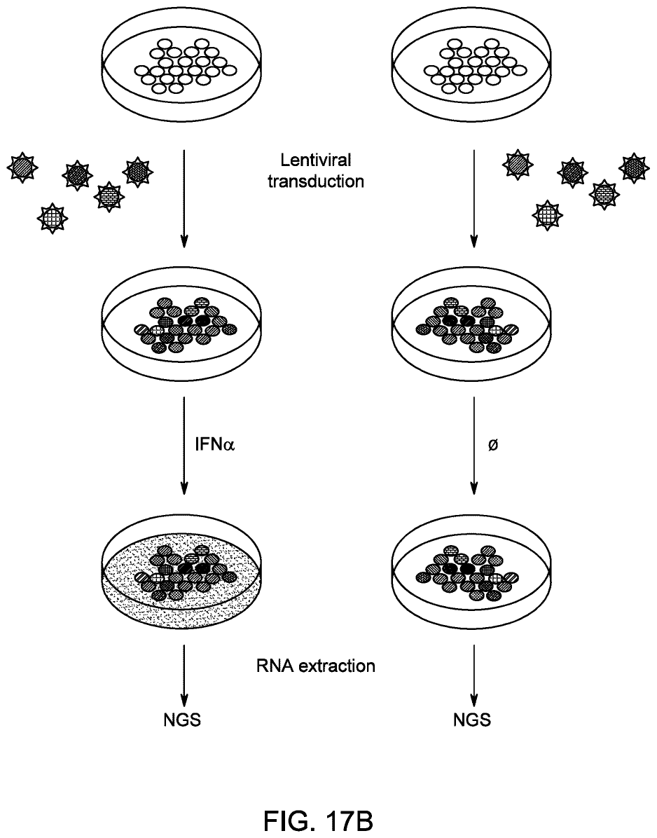

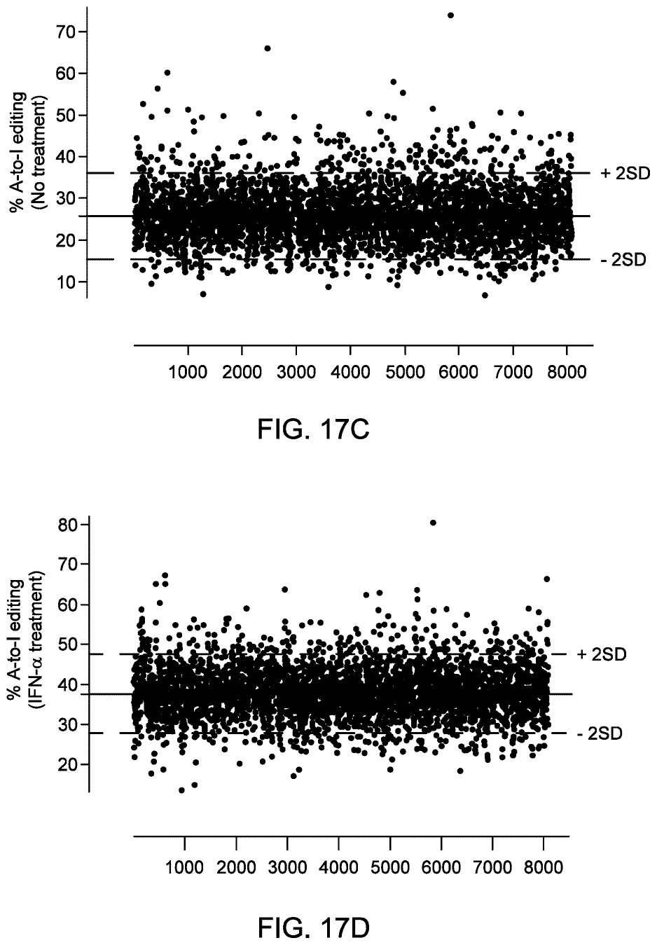

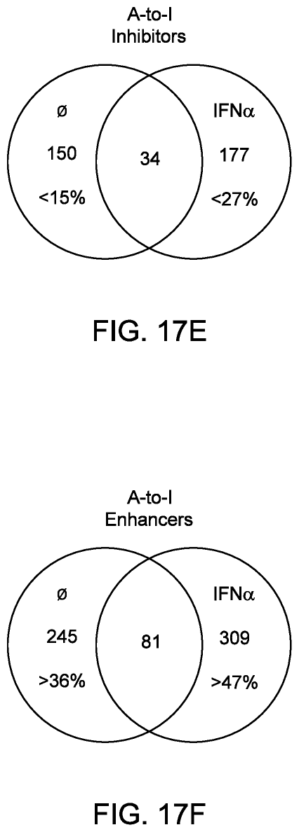

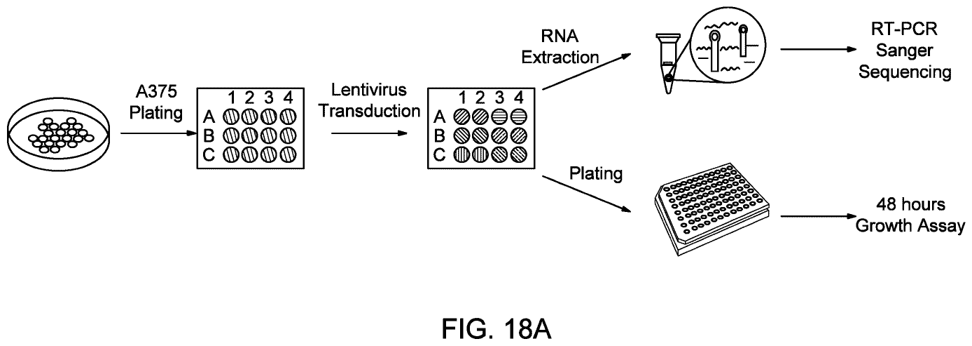

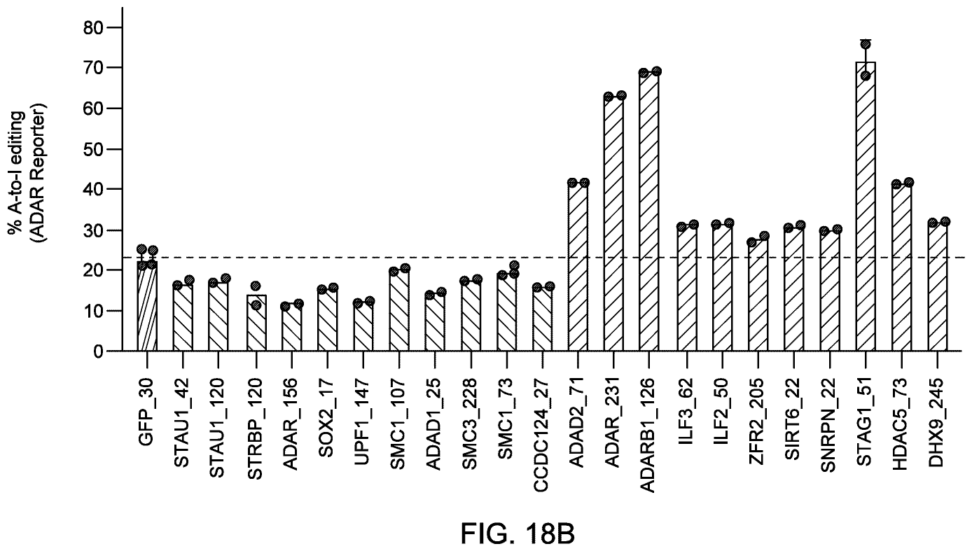

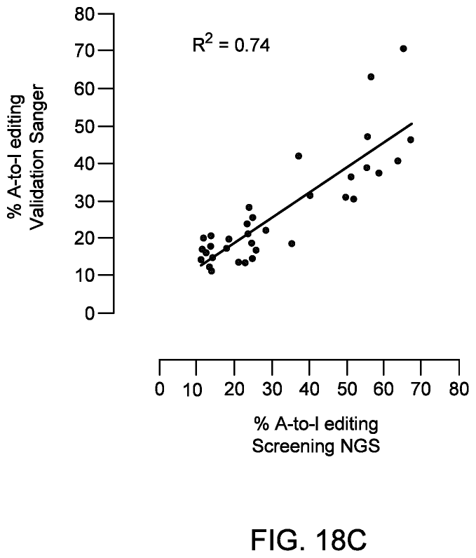

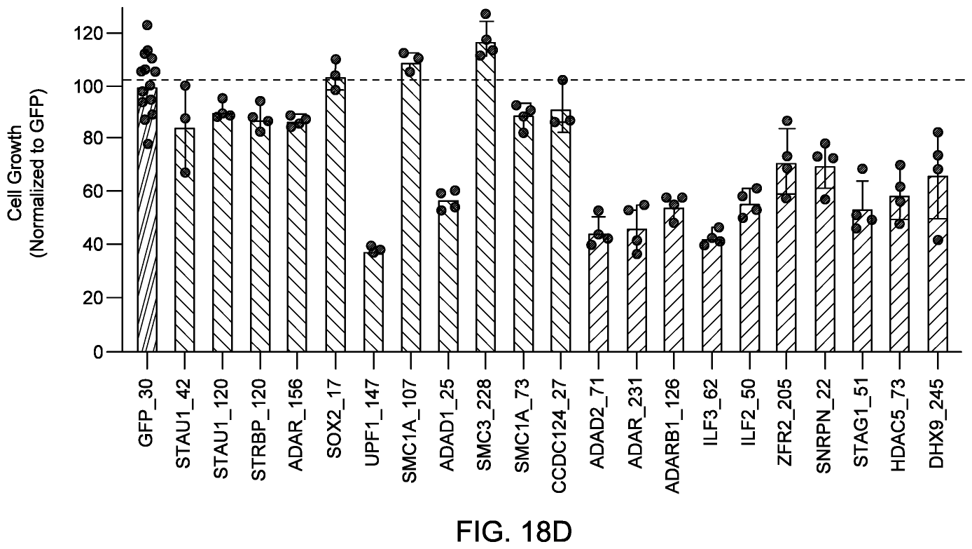

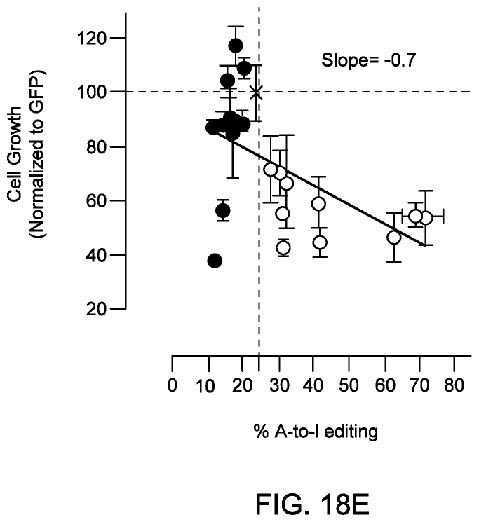

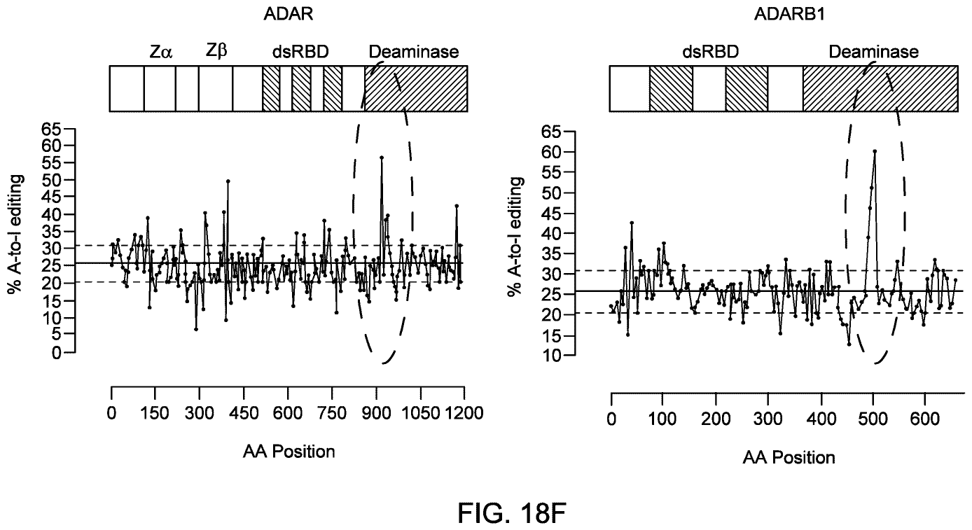

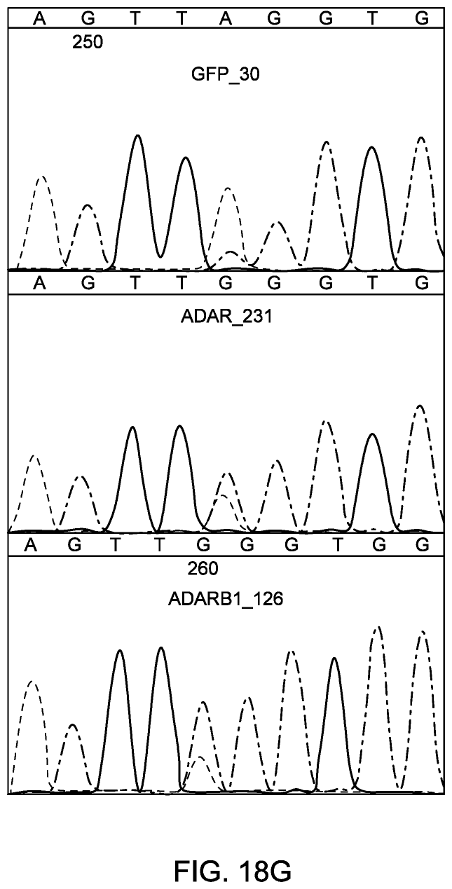

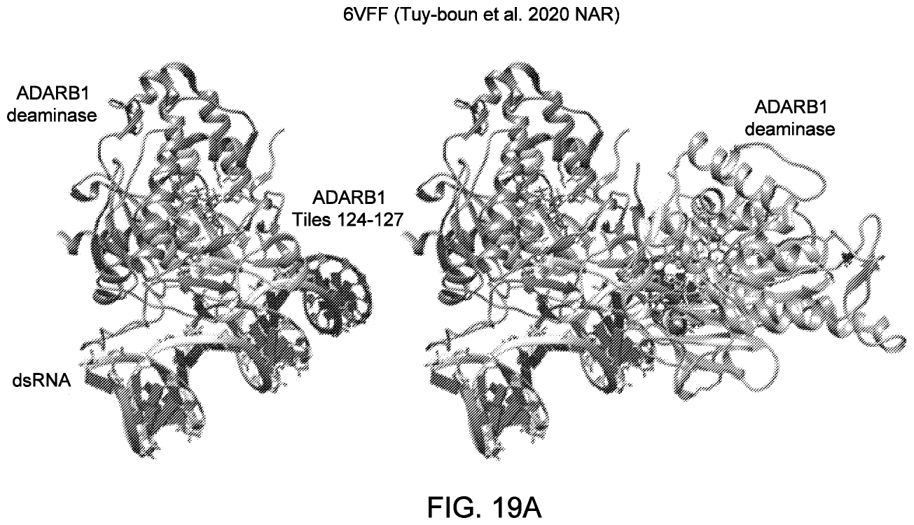

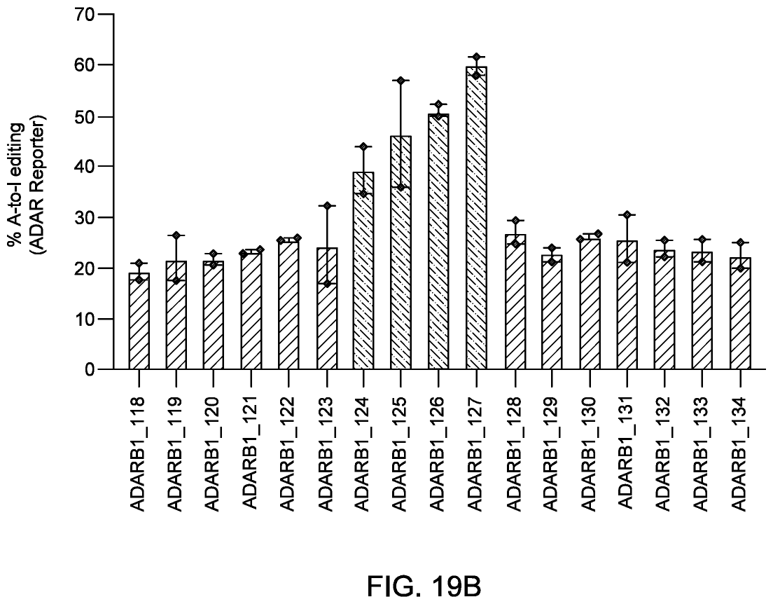

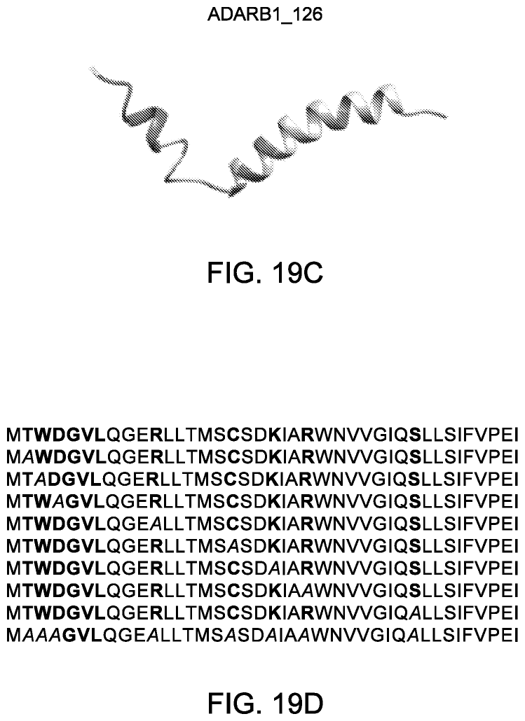

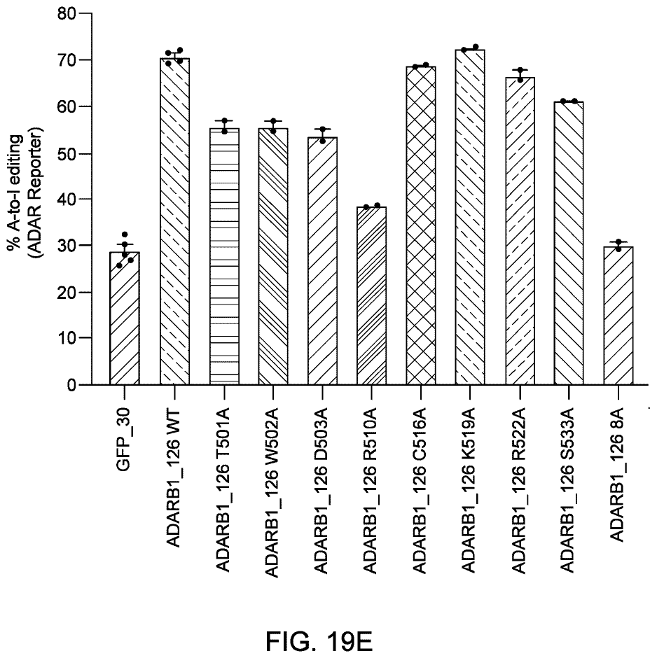

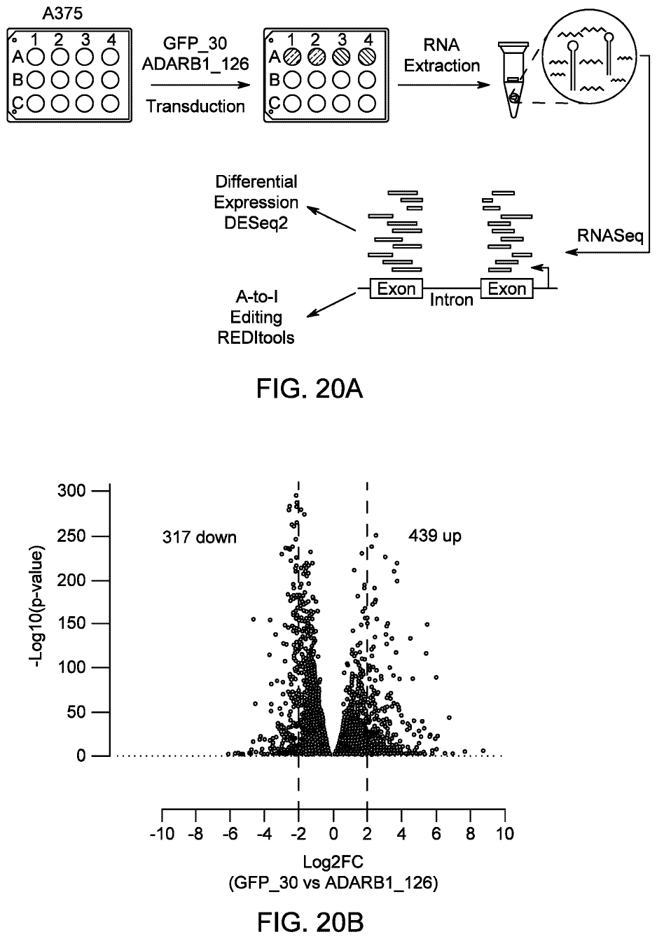

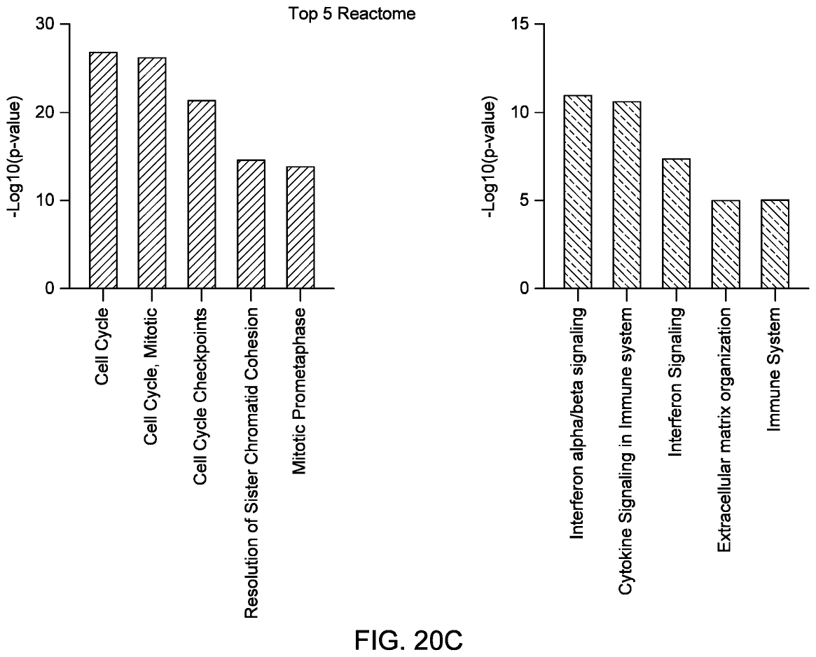

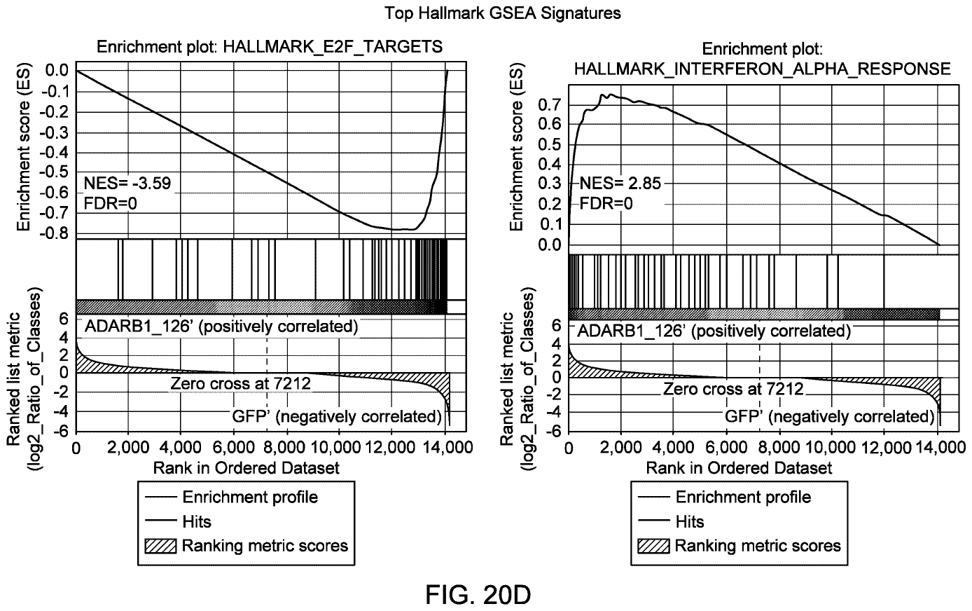

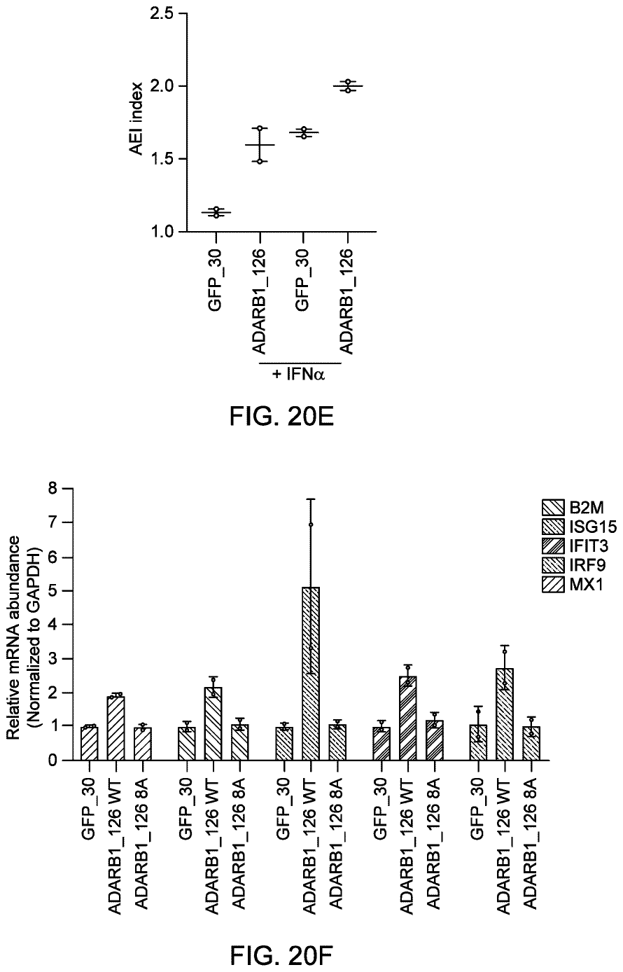

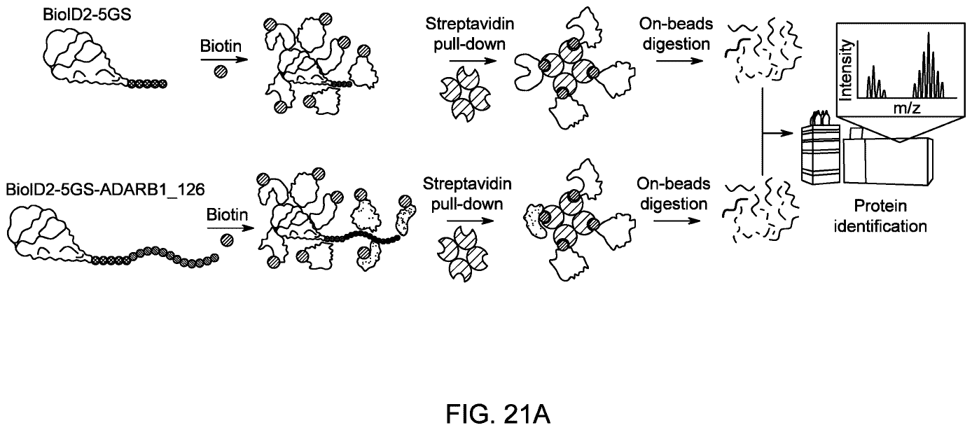

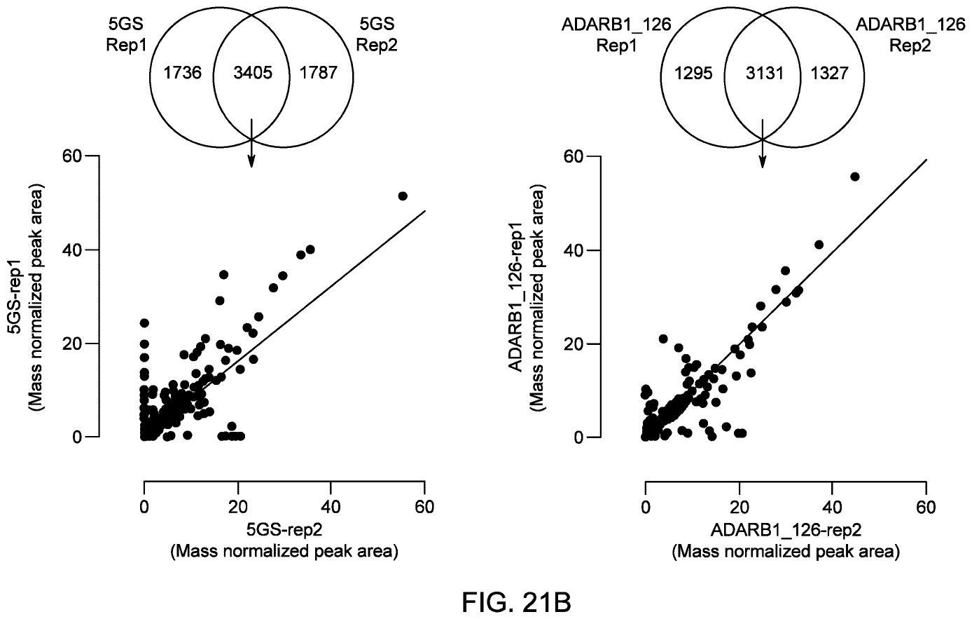

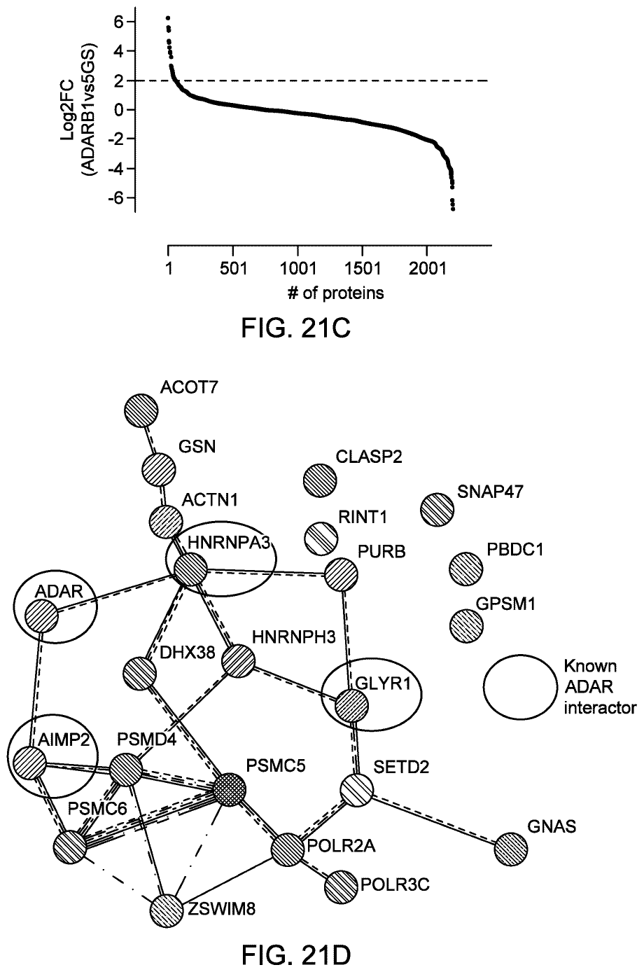

Provided herein is a screening methodology to measure the effect of small peptide on A-to-I RNA editing in situ and screen thousands of peptides in a single experiment. First, a library of peptides with the potential of impairing A-to-I editing was designed and built. ADAR enzymes edit RNA only on double-stranded RNA structures and via ADAR's double stranded RNA-Binding Domain (dsRBD). Interestingly, it has been shown that other proteins containing dsRBD can compete with ADAR and have antagonistic effects on RNA editing, and apoptosis. The A-to-I PepTile library is composed of 19 Human proteins having dsRBD and 34 known protein-protein interactors of ADAR1/2. All these proteins were tiled into 40-mer peptides every 4 amino acids (FIG. 12A). To measure A-to-I editing, a biosensor consisting of a 71 bp dsRNA hairpin structure that gets A-to-I edited at a rate of 25% in A375 melanoma cells, and ~37% upon IFNα treatment was used. This A-to-I editing reporter is fused with DNA sequence encoding for a peptide from the A-to-I PepTile library. The A-to-I PepTile plasmid pool (10304 elements) was used to produced lentiviruses and transduced A375 cells at a low multiplicity of infection (MOI) (FIG. 12B). A part of the infected cells was treated with IFNα at 6000 U/ml for 48 hours, and then bulk RNA of the basal and treated condition was harvested. RNA was then retrotranscribed into cDNA and sequenced by Next-Generation Sequencing (NGS). With the sequencing data, the A-to-I editing percentage of the RNA reporter for each PepTile is calculated since both elements are encoded on the same transcript. Moreover, with this screen one can also measure the abundance of the PepTiles between the untreated and treated conditions and determine Fitness score associated to every PepTile. To ensure a robust and more accurate A-to-I editing % estimation a cut-off of 100 read counts was set for each PepTile. Out of 8088 peptides the RNA reporter is edited at a rate of 25.75%+/−6% in basal condition and 37.6%+/−6% upon IFNα treatment (FIG. 12C). To select hits, a cut-off at 2-fold the standard deviation was set and found 86 PepTiles that significantly perturbed A-to-I editing in both basal and IFNα conditions (Table 4). SEQ ID NOs: 2125-2147 are A-to-I peptide inhibitors and SEQ ID NOs: 2148-2210 are A-to-I activators. In addition, 117 PepTiles were identified that either synergize (SEQ ID NOs: 2211-2243) or protect (SEQ ID NOs: 2244-2327) to IFNα-induced cell death (FIG. 12D and Table 5). These PepTiles are potential new therapeutic agents that can modulate the Interferon pathway. They may be beneficial in treating inflammatory conditions and may also enhance the effectiveness of cancer immunotherapy through synergistic effects.

### Example 2

Inhibitory peptides have immense potential as both research tools and therapeutics. Direct inhibition of protein activity without genetic alteration opens unique screening avenues with which to probe protein function. For example, protein-protein interaction networks could be more precisely perturbed via inhibitory peptides contacting a specific protein surface in contrast to complete genetic knockdown. The ability to identify protein regions associated with cell fitness can also serve to complement traditional drug development efforts, such as through determining critical residues for inhibition via small molecules or antibodies. Additionally, this screening method identifies inhibitory peptides that are immediately translatable, bypassing the need for additional high-throughput screens to identify candidate molecules.

Many proteins can be inhibited in a dominant negative fashion by short peptides/proteins derived from their own wild type coding sequence. Leveraging this fact, the method described herein provides compositions and methods that use a comprehensive lentiviral library of gene fragments tiling key oncogenes via a highly modular oligonucleotide synthesis protocol. This library was then used to conduct a pooled fitness screen, mining novel peptide motifs.

The screening strategy presented herein can probe protein functional regions with single amino acid resolution and can be readily scalable due to the ease of array-based oligonucleotide synthesis. In the exemplary studies presented herein, the platform-based screening methodology disclosed can identify peptide inhibitors of known oncogenes, including proteins, such as KRAS, that are a challenge to target using small molecule therapeutics.

As such, the screening methodology provided herein enables rapid discovery of potentially translatable peptide therapeutics and can be easily adaptable to diverse target genes and pathogenic contexts. These pathogenic contexts are not limited to cancer cell fitness.

This methodology can be compatible with screening for higher level phenotypes via FACS, scRNAseq, as well as functional assays.

The method described herein provides synthesis of a gene fragment library using methods known to those of skill in the art. Gene fragments are then delivered to cells, using methods known to those in the art, including viral and non-viral methods. For example, the gene fragments are delivered to cells using lentiviral transduction. The method provides that the cells may be cultured, such as under selective pressure and lysed and sequenced, such as deep-sequencing may be performed, to identify one or more nucleic acids from the library that are introduced into the cells using methods known to those of skill in the art, such as those described in Shalem, O., Sanjana, N. E. & Zhang, F., Nat Rev Genet 16:299-311 (2015); Shalem, O. et al., Science 343, 84-87 (2014); Agrotis, A. & Ketteler, R., Front Genet 6:300, each of which is hereby incorporated by reference in its entirety.

Statistically-overrepresented nucleic acids may be determined using methods associated with pooled and arrayed screens (See Agrotis, A. & Ketteler, R. A new age in functional genomics using CRISPR/Cas9 in arrayed library screening. Front Genet 6, 300 (201S) hereby incorporated by reference in its entirety).

A gene fragment library coding for potentially inhibitor peptides is synthesized via pooled oligonucleotide synthesis using solid-phase synthesis. Solid-phase synthesis is typically carried out on a solid support held between filters, in columns that enable all reagents and solvents to pass through freely. Solid-phase synthesis has a number of advantages over solution synthesis, including, large excesses of solution-phase reagents can be used to drive reactions quickly to completion; impurities and excess reagents are washed away and no purification may be required after each step; and the process is amenable to automation on computer-controlled solid-phase synthesizers. Solid supports (also called resins) are the insoluble particles, typically 50-200 μm in diameter, to which the oligonucleotide can be bound during synthesis. Many types of solid support have been used, but controlled pore glass (CPG) and polystyrene have proved to be the most useful.

The phosphoramidite method, pioneered by Marvin Caruthers in the early 1980s, and enhanced by the application of solid-phase technology and automation, is typically used. Phosphoramidite oligo synthesis proceeds in the 3′- to 5′-direction (opposite to the 5′- to 3′-direction of DNA biosynthesis in DNA replication). One nucleotide can be added per synthesis cycle. At the beginning of oligonucleotide synthesis, the first protected nucleoside can be pre-attached to the resin and the operator selects an A, G, C or T synthesis column depending on the nucleoside at the 3′-end of the desired oligonucleotide. The support-bound nucleoside has a 5′-DMT protecting group (DMT=4,4′-dimethoxytrityl), the role of which is to prevent polymerization during resin functionalization, and this protecting group must be removed (detritylation) from the support-bound nucleoside before oligonucleotide synthesis can proceed.

Following detritylation, the support-bound nucleoside is ready to react with the next base, which can be added in the form of a nucleoside phosphoramidite monomer. A large excess of the appropriate nucleoside phosphoramidite can be mixed with an activator (tetrazole or a derivative), both of which are dissolved in acetonitrile (a good solvent for nucleophilic displacement reactions). The diisopropylamino group of the nucleoside phosphoramidite can be protonated by the activator, and can be thereby converted to a good leaving group. It can be rapidly displaced by attack of the 5′-hydroxyl group of the support-bound nucleoside on its neighboring phosphorus atom, and a new phosphorus-oxygen bond can be formed, creating a support-bound phosphite trimester. Nucleoside phosphoramidites are reasonably stable in an inert atmosphere and can be prepared in large quantities, shipped around the world and stored as dry solids for several months prior to use. Only upon protonation do nucleoside phosphoramidites become reactive.

It may not be unreasonable to expect a yield of 99.5% during each coupling step, but even with the most efficient chemistry and the purest reagents it may not be possible to achieve 100% reaction of the support-bound nucleoside with the incoming phosphoramidite. This means that there will be a few unreacted 5′-hydroxyl groups on the resin-bound nucleotide; if left unchecked, these 5′-hydroxyl groups would be available to partake in the next coupling step, reacting with the incoming phosphoramidite. The resulting oligonucleotide would lack one base. Deletion mutations are avoided by introducing a “capping” step after the coupling reaction, to block the unreacted 5′-hydroxyl groups. Two capping solutions are used on the synthesizer: acetic anhydride and N-methylimidazole (NMI). These two reagents (dissolved in tetrahydrofuran with the addition of a small quantity of pyridine) are mixed on the DNA synthesizer prior to delivery to the synthesis column. The electrophilic mixture rapidly acetylates alcohols, and the pyridine ensures that the pH remains basic to prevent detritylation of the nucleoside phosphoramidite by the acetic acid formed by reaction of acetic anhydride with NMI. Acetylation of the 5′-hydroxyl groups renders them inert to subsequent reactions.

After phosphoramidite coupling, capping and oxidation, the DMT protecting group at the 5′-end of the resin-bound DNA chain are removed so that the primary hydroxyl group can react with the next nucleotide phosphoramidite. Deprotection with trichloroacetic acid in dichloromethane can be rapid and quantitative. An orange color can be produced by cleaved DMT carbocation, which absorbs in the visible region at 495 nm. The intensity of this absorbance is used to determine the coupling efficiency. Most commercially available DNA synthesizers have hardware to measure and record the trityl yield for each cycle so that the efficiency of synthesis can be monitored in real time. The cycle can be repeated, once for each base, to produce the required oligonucleotide. The linker can be a chemical entity that attaches the 3′-end of the oligonucleotide to the solid support. It must be stable to all the reagents used in solid-phase oligonucleotide assembly, but cleavable under specific conditions at the end of the synthesis. The cleavage reaction can be carried out automatically on some synthesizers, and the ammoniacal solution containing the oligonucleotide can be delivered to a glass vial. Alternatively, the cleavage can be carried out manually by taking the column off the synthesizer and washing it with syringes containing ammonium hydroxide. The oligonucleotide, now dissolved in concentrated aqueous ammonia, can be heated to remove the protecting groups from the heterocyclic bases and phosphates. The aqueous solution can then be removed by evaporation and the oligonucleotides are ready for purification.

Gene fragment libraries can be generated by splitting target gene(s) or relevant protein interaction partners into defined size fragments (e.g., 100 to 150 nt fragments). Several different commercial platforms exist for producing pooled oligonucleotides (e.g., CustomArray, Twist Bioscience, Agilent Technologies). Site directed mutagenesis can also be performed to optimize gene fragments for particular applications. Additionally, computational structure guided library design can be used to construct de novo gene fragment libraries which have predicted binding to target genes. The gene oligonucleotides are synthesized as gene fragment library which includes a plurality of gene fragments having more than 50, 100, 200, 300, 400, 500, 600, 700, 800, 1000, 1500, 2000, 5000, 10000 different sequences based from one or more target genes. Examples of target genes includes genes associated with a disease or disorder condition, such as oncogenes, or targets of infectious agents. The screening methodology provided herein can be agnostic to the disease model chosen. In theory, peptide inhibitors (drug-like peptides) of any protein target can be identified using the screening methods disclosed herein. The method can be assisted by having an in vitro assay to select for the phenotype of interest when contacted with a drug-like peptide. Alternatively, novel dominant negative inhibitors of protein function could be produced and sold as scientific reagents. Peptides across a wide size range (5-60 AA) can be screened via the screening methods disclosed herein, highlighting the broad utility of the screening methods provided herein as a platform technology.

Once synthesized, oligonucleotides are cloned into the appropriate vector for the biological model and planned screening study. Viral vectors are introduced at a low multiplicity of infection (MOI) in order to ensure that cells receive only a single gene fragment from the library pool.

The frequency of observing any particular tag (gene fragment) before and after phenotypic selection can be a parameter measured using the screening methods described herein. After cloning, the distribution of cloned oligos in the pooled library can be assessed using Next-Generation Sequencing (NGS). In some cases, high throughput sequencing is then used to track the abundance of each gene fragment as the target cells grow. Gene fragments which significantly deplete over the course of cell growth are deleterious to cell fitness and can then be synthesized individually to measure their performance in additional assays, such as cell proliferation, RNA-Seq, Mass Spec, etc.

As exemplified in the studies described herein, the screening methodology provided herein allowed for the identification of highly effective dominant negative inhibitors of oncogene mediated cancer cell proliferation and viral entry. Because the library of gene fragments can be user defined and custom synthesized, the screening methods disclosed herein are easily adaptable to diverse projects where a selection strategy can be devised to enrich or deplete cells with the phenotype of interest.

Technologies which can partition cells based on unique phenotypic features such as Flow Activated Cell Sorting (FACS), or magnetic bead pull down will play a key role in expanding this methodology beyond proliferation. This combined with the decreasing cost of single cell RNA sequencing allows for investigation of increasingly more complex phenotypes, such as cellular differentiation and regeneration. Dominant negative peptides have immense potential as both research tools and therapeutics. Direct inhibition of protein activity without genetic alteration opens unique screening avenues with which to probe protein function. For example, one of the dominant negative fragments identified in the studies presented herein appears selective to a specific KRAS mutation, potentially allowing for mutant allele specific inhibition in a functional screen. Furthermore, with minimal engineering, a dominant negative peptide EGFR inhibitor opposed TNBC cell growth in vitro as effectively as FDA approved small molecules. Because one of the key limitations of dominant negative peptide inhibitors can be the intracellular location of the target proteins, it is expected that advances in biologics delivery can improve the translational relevance this strategy.

In concert with direct oncological applications, the screening methodologies provided herein can be used to identify peptides which are immunostimulatory/immunosuppressive via FACS based screening of immune effector cells. The design of the experiment could be tailored specifically to the pathogenic context (e.g., screening in regulatory T cells to identify immunosuppressive peptides to treat autoimmune disorders). Furthermore, the screening methodologies disclosed herein could also be adapted to inhibiting pathogenic protein-protein interactions directly, such as neurodegenerative tauopathies.

### Example 2: Pan-Cancer PiTile Screen in Metastatic Melanoma

We built a

[symbol]

### Example 3: Pan-Cancer PiTile Screen in Metastatic Melanoma

We built a DNA-encoded peptide library composed of all known Human oncogenes and Tumor Suppressors that we fragmented in 40 amino acid long peptides, the Pan-Cancer library. Using lentiviral expression system, we transduced the Pan-Cancer library in A375 cell line. After 14 days of growth, the frequency of each peptide is measured by NGS and this reveal which peptides affect cell fitness. Over the 92144 peptides tested, we identified 3201 (3.5%) peptides that significantly decrease cell fitness (Fitness score<−2 z-score). To select peptides hits for further investigations, we mapped the signal over the original amino acid positions of the derived protein and look for successive peptide fragments decreasing cell fitness. To develop drug-like peptides, we prioritize αhelical peptides derived from known protein-protein interaction domains and with existing crystal structures. Then we compute biophysical properties (Isoelectric point, Helicity, Hydrophobicity) of all possible 15-mer peptides derived from the region of interest. To potentiate cell penetrating capacity of the chosen helix, we design staple peptide versions that will also constrain αhelical conformation. To determine the position of the staple we leverage information from crystal structures to not perturb critical molecular interaction with the targeted protein. Designed stapled peptides are tested by dose-response using 48-hour cell growth assay as a readout. Bioactive staple peptides are characterized for on target activity via various biochemical and functional approaches.

### Case of RAF1:

One compelling peptidic region was one derived from RAF1 Ras-Binding Domain (RBD) which was identified as hit in A375 as well in previous screening in MDA-MB-231 and Hs578T. We analyzed several crystal structures of RAF1 RBD interacting with RAP1A (1C1Y), HRAS (4G0N), KRAS (6XHA) which describe the critical residues mediating interactions on a short αhelix (13 amino acids). To enable cell penetrating capabilities, we designed a RAF1 derived hydrocarbon-staple (i,i+7) peptide taking in consideration these critical residues for protein-protein interaction. We tested activity of this peptide by dose-response on a 48-hour cell growth assay in A375 and MDA-MB-231. The RAF1 stapled peptide led to a complete cell death in both cell line with an IC₅₀ of 16 to 21 μM respectively in A375 and MDA-MB-231. We analyzed transcriptomic signature of A375 treated with stapled RAF1 peptide for 24 hours by RNA-sequencing. We found that RAS_Pathway (NES=−1.65), RAF_Activation (NES=−1.48), MAPK_Pathway (NES=−1.23) were downregulated in cells treated with stapled RAF1 peptide.

### Example 4: A-to-I RNA Editing PiTile (REPTILE) Screen in Metastatic Melanoma

Adenosine-to-inosine (A-to-I) RNA editing is a post-transcriptional modification process that involves the conversion of adenosine to inosine in RNA molecules. This process is catalyzed by the adenosine deaminase acting on RNA (ADAR) family of enzymes and can result in changes to the RNA sequence, structure, and function. Recent studies have shown that A-to-I RNA editing plays a crucial role in cancer progression and tumorigenesis. For example, ADAR-mediated editing of microRNA (miRNA) binding sites in messenger RNAs (mRNAs) can lead to altered gene expression and contribute to cancer development. Targeting A-to-I RNA editing has emerged as a potential strategy for cancer therapy.

Additionally, A-to-I RNA editing has been shown to affect the stability and activity of various RNA molecules, including long non-coding RNAs (lncRNAs) and circular RNAs (circRNAs), which can promote or inhibit cancer growth. Several studies have demonstrated that inhibition of ADAR enzymes can reduce tumor growth and sensitize cancer cells to chemotherapy and immunotherapy. For example, two recent studies have showed that ADAR1 inhibition enhanced the efficacy of anti-PD-1 therapy in a mouse model of melanoma and lung adenocarcinoma [1,2]. A-to-I RNA editing can influence the response of cancer cells to immunotherapy. For example, A-to-I editing of IFNAR1, the receptor for type I IFNs, can impair the IFN signaling pathway and promote cancer cell survival [3]. a study by Karki et al. [4] found that ADAR1 promote tumorigenesis via innate immune response and interferon pathway. Given the role of A-to-I RNA editing in the Interferon pathway and immune checkpoint regulation, targeting this process has emerged as a potential strategy for cancer immunotherapy. Inhibition of ADAR enzymes have been shown to enhance the response of cancer cells to IFN therapy and immune checkpoint blockade. For example, a study by Wang et al. [5] found that ADAR1 inhibition sensitized melanoma cells to IFN therapy and increased T cell infiltration in tumors. Thus, Peptide that perturb A-to-I editing has the potential to enhance immunotherapy in melanoma.
[1] Ishizuka, J. J., Manguso, R. T., Cheruiyot, C. K., Bi, K., Panda, A., Iracheta-Vellve, A., . . . & Haining, W. N. (2019). Loss of ADAR1 in tumours overcomes resistance to immune checkpoint blockade. Nature, 565 (7737), 43-48.
[2] Gannon, H. S., Zou, T., Kiessling, M. K., Gao, G. F., Cai, D., Choi, P. S., . . . & Meyerson, M. (2018). Identification of ADAR1 adenosine deaminase dependency in a subset of cancer cells. Nature communications, 9 (1), 5450.
[3] Tomaselli, S., Galeano, F., Locatelli, F., & Gallo, A. (2015). ADARs and the balance game between virus infection and innate immune cell response. Current issues in molecular biology, 17 (1), 37-52.
[4] Karki, R., Sundaram, B., Sharma, B. R., Lee, S., Malireddi, R. S., Nguyen, L. N., . . . & Kanneganti, T. D. (2021). ADAR1 restricts ZBP1-mediated immune response and PANoptosis to promote tumorigenesis. Cell reports, 37 (3), 109858
[5] Wang, H., Chen, S., Wei, J., Song, G., & Zhao, Y. (2021). A-to-I RNA editing in cancer: from evaluating the editing level to exploring the editing effects. Frontiers in Oncology, 10, 632187

### Informal Sequence Listing

TABLE 3
PanCancer Peptide Table

| | | | AA | | | |
|---|---|---|---|---|---|---|
| SEQ | | | Start | | | |
| ID | Driver | Peptide | posi- | | Fitness | |
| NO: | ID | ID | tion | AA sequence | score | FDR |
| 1. | ABL1 | ABL1_57 | 336 | MMTYGNLLDYLRECNRQEVNAVVLL | −2.98 | 8.17E−03 |
| | | | | YMATQISSAMEYLEKK | | |
| 2. | ABL1 | ABL1_62 | 366 | MSSAMEYLEKKNFIHRDLAARNCLV | −2.31 | 3.28E−02 |
| | | | | GENHLVKVADFGLSRL | | |
| 3. | ABL1 | ABL1_63 | 372 | MLEKKNFIHRDLAARNCLVGENHLV | −1.90 | 4.38E−02 |
| | | | | KVADFGLSRLMTGDTY | | |
| 4. | ABL1 | ABL1_65 | 384 | MARNCLVGENHLVKVADFGLSRLMT | −2.51 | 2.14E−02 |
| | | | | GDTYTAHAGAKFPIKW | | |
| 5. | AFDN | AFDN_98 | 582 | MLILPASIEFRESSEDSFLSAIINY | −2.24 | 4.44E−02 |
| | | | | TNSSTVHFKLSPTYVL | | |
| 6. | AFDN | AFDN_99 | 588 | MIEFRESSEDSFLSAIINYTNSSTV | −2.19 | 4.63E−02 |
| | | | | HFKLSPTYVLYMACRY | | |
| 7. | AFDN | AFDN_100 | 594 | MSEDSFLSAIINYTNSSTVHFKLSP | −1.88 | 7.06E−02 |
| | | | | TYVLYMACRYVLSNQY | | |
| 8. | AFDN | AFDN_101 | 600 | MSAIINYTNSSTVHFKLSPTYVLYM | −3.24 | 5.43E−03 |
| | | | | ACRYVLSNQYRPDISP | | |
| 9. | AFDN | AFDN_234 | 1398 | MHQRWYEKEKARLEEERERKRREQE | −4.02 | 1.04E−03 |
| | | | | RKLGQMRTQSLNPAPF | | |
| 10. | AFDN | AFDN_235 | 1404 | MKEKARLEEERERKRREQERKLGQM | −4.06 | 9.58E−04 |
| | | | | RTQSLNPAPFSPLTAQ | | |
| 11. | AFF1 | AFF1_175 | 1044 | MKIFAVLCMRCQSILNMAMFRCKKD | −1.90 | 7.33E−02 |
| | | | | IAIKYSRTLNKHFESS | | |
| 12. | AFF1 | AFF1_176 | 1050 | MCMRCQSILNMAMFRCKKDIAIKYS | −2.48 | 3.00E−02 |
| | | | | RTLNKHFESSSKVAQA | | |
| 13. | AFF4 | AFF4_42 | 246 | MNSMLQKPTAYVRPMDGQESMEPKL | −2.45 | 1.61E−02 |
| | | | | SSEHYSSQSHGNSMTE | | |
| 14. | AFF4 | AFF4_43 | 252 | MPTAYVRPMDGQESMEPKLSSEHYS | −3.86 | 1.44E−03 |
| | | | | SQSHGNSMTELKPSSK | | |
| 15. | AGAP2 | AGAP2_25 | 144 | MLEDENSFQAVSRLHGQLSSLRGEG | −4.05 | 9.69E−04 |
| | | | | RGGLALALVGTQDRIS | | |
| 16. | AGAP2 | AGAP2_26 | 150 | MFQAVSRLHGQLSSLRGEGRGGLAL | −4.18 | 7.27E−04 |
| | | | | ALVGTQDRISASSPRV | | |
| 17. | AGR2 | AGR2_5 | 24 | MVKPGAKKDTKDSRPKLPQTLSRGW | −4.01 | 7.98E−04 |
| | | | | GDQLIWTQTYEEALYK | | |
| 18. | AGR2 | AGR2_6 | 30 | MKDTKDSRPKLPQTLSRGWGDQLIW | −4.14 | 7.90E−04 |
| | | | | TQTYEEALYKSKTSNK | | |
| 19. | AGR2 | AGR2_12 | 66 | MTSNKPLMIIHHLDECPHSQALKKV | −1.81 | 7.72E−02 |
| | | | | FAENKEIQKLAEQFVL | | |
| 20. | AGR2 | AGR2_13 | 72 | MMIIHHLDECPHSQALKKVFAENKE | −2.19 | 4.93E−02 |
| | | | | IQKLAEQFVLLNLVYE | | |
| 21. | AIM2 | AIM2_32 | 186 | MFFFVKVFNTLLKDKFIPKRIIIIA | −3.35 | 3.77E−03 |
| | | | | RYYRHSGFLEVNSASR | | |
| 22. | AIM2 | AIM2_33 | 192 | MFNTLLKDKFIPKRIIIIARYYRHS | −3.21 | 5.76E−03 |
| | | | | GFLEVNSASRVLDAES | | |
| 23. | AIM2 | AIM2_34 | 198 | MDKFIPKRIIIIARYYRHSGFLEVN | −3.41 | 3.40E−03 |
| | | | | SASRVLDAESDQKVNV | | |
| 24. | AJUBA | AJUBA_50 | 294 | MAGARGEPSGIEPSGLEEPPGPFVP | −3.46 | 3.12E−03 |
| | | | | EAARARMREPEAREDY | | |
| 25. | AJUBA | AJUBA_52 | 306 | MSGLEEPPGPFVPEAARARMREPEA | −1.85 | 8.22E−02 |
| | | | | REDYFGTCIKCNKGIY | | |
| 26. | ALB | ALB_66 | 390 | MHECYAKVFDEFKPLVEEPQNLIKQ | −2.25 | 4.52E−02 |
| | | | | NCELFEQLGEYKFQNA | | |
| 27. | ALB | ALB_68 | 402 | MPLVEEPQNLIKQNCELFEQLGEYK | −2.13 | 5.43E−02 |
| | | | | FQNALLVRYTKKVPQV | | |
| 28. | ALB | ALB_75 | 444 | MPTLVEVSRNLGKVGSKCCKHPEAK | −2.48 | 1.78E−02 |
| | | | | RMPCAEDYLSVVLNQL | | |
| 29. | ALB | ALB_76 | 450 | MSRNLGKVGSKCCKHPEAKRMPCAE | −2.47 | 1.51E−02 |
| | | | | DYLSVVLNQLCVLHEK | | |
| 30. | ALB | ALB_77 | 456 | MVGSKCCKHPEAKRMPCAEDYLSVV | −2.77 | 9.67E−03 |
| | | | | LNQLCVLHEKTPVSDR | | |
| 31. | ALB | ALB_79 | 468 | MRMPCAEDYLSVVLNQLCVLHEKTP | −4.27 | 5.99E−04 |
| | | | | VSDRVTKCCTESLVNR | | |
| 32. | ALK | ALK_122 | 726 | MKVPATDTYSISGYGAAGGKGGKNT | −5.14 | 5.83E−05 |
| | | | | MMRSHGVSVLGIFNLE | | |
| 33. | ALK | ALK_124 | 738 | MYGAAGGKGGKNTMMRSHGVSVLGI | −5.50 | 2.03E−05 |
| | | | | FNLEKDDMLYILVGQQ | | |
| 34. | ALK | ALK_126 | 750 | MMMRSHGVSVLGIFNLEKDDMLYIL | −1.94 | 2.21E−02 |
| | | | | VGQQGEDACPSTNQLI | | |
| 35. | ALK | ALK_129 | 768 | MDMLYILVGQQGEDACPSTNQLIQK | −2.05 | 4.84E−02 |
| | | | | VCIGENNVIEEEIRVN | | |
| 36. | ALK | ALK_151 | 900 | MTGGHSCPQAMKKWGWETRGGFGGG | −4.00 | 1.07E−03 |
| | | | | GGGCSSGGGGGGYIGG | | |
| 37. | ALK | ALK_153 | 912 | MWGWETRGGFGGGGGGCSSGGGGGG | −3.16 | 5.26E−03 |
| | | | | YIGGNAASNNDPEMDG | | |
| 38. | ANAPC15 | ANAPC15_12 | 66 | MEEDDEDDEDSEEDSEDDEDMQDMD | −4.15 | 7.83E−04 |
| | | | | EMNDYNESPDDGEVNE | | |
| 39. | ANAPC15 | ANAPC15_13 | 72 | MDEDSEEDSEDDEDMQDMDEMNDYN | −4.14 | 8.02E−04 |
| | | | | ESPDDGEVNEVDMEGN | | |
| 40. | APC | APC_84 | 498 | MRYAGMALTNLTFGDVANKATLCSM | −1.97 | 3.21E−02 |
| | | | | KGCMRALVAQLKSESE | | |
| 41. | APC | APC_88 | 522 | MKGCMRALVAQLKSESEDLQQVIAS | −1.96 | 3.27E−02 |
| | | | | VLRNLSWRADVNSKKT | | |
| 42. | APC | APC_89 | 528 | MLVAQLKSESEDLQQVIASVLRNLS | −2.01 | 3.06E−02 |
| | | | | WRADVNSKKTLREVGS | | |
| 43. | APC | APC_91 | 540 | MQQVIASVLRNLSWRADVNSKKTLR | −2.17 | 2.43E−02 |
| | | | | EVGSVKALMECALEVK | | |
| 44. | APC | APC_127 | 756 | MQKALEAELDAQHLSETFDNIDNLS | −2.39 | 3.58E−02 |
| | | | | PKASHRSKQRHKQSLY | | |
| 45. | APC | APC_128 | 762 | MELDAQHLSETFDNIDNLSPKASHR | −2.71 | 2.10E−02 |
| | | | | SKQRHKQSLYGDYVFD | | |
| 46. | APC | APC_130 | 774 | MNIDNLSPKASHRSKQRHKQSLYGD | −2.53 | 2.84E−02 |
| | | | | YVFDTNRHDDNRSDNF | | |
| 47. | APC | APC_131 | 780 | MPKASHRSKQRHKQSLYGDYVFDTN | −2.15 | 4.66E−02 |
| | | | | RHDDNRSDNFNTGNMT | | |
| 48. | APC | APC_155 | 924 | MSSAAHTHSNTYNFTKSENSNRTCS | −2.05 | 5.72E−02 |
| | | | | MPYAKLEYKRSSNDSL | | |
| 49. | APC | APC_158 | 942 | MSNRTCSMPYAKLEYKRSSNDSLNS | −2.14 | 5.00E−02 |
| | | | | VSSSDGYGKRGQMKPS | | |
| 50. | APC | APC_160 | 954 | MEYKRSSNDSLNSVSSSDGYGKRGQ | −2.01 | 4.95E−02 |
| | | | | MKPSIESYSEDDESKF | | |
| 51. | APC | APC_161 | 960 | MNDSLNSVSSSDGYGKRGQMKPSIE | −1.81 | 6.67E−02 |
| | | | | SYSEDDESKFCSYGQY | | |
| 52. | APC | APC_162 | 966 | MVSSSDGYGKRGQMKPSIESYSEDD | −1.82 | 6.17E−02 |
| | | | | ESKFCSYGQYPADLAH | | |
| 53. | APC | APC_169 | 1008 | MHSANHMDDNDGELDTPINYSLKYS | −1.80 | 4.08E−02 |
| | | | | DEQLNSGRQSPSQNER | | |
| 54. | APC | APC_189 | 1128 | MLCQEDDYEDDKPTNYSERYSEEEQ | −1.98 | 3.16E−02 |
| | | | | HEEEERPTNYSIKYNE | | |
| 55. | APC | APC_190 | 1134 | MYEDDKPTNYSERYSEEEQHEEEER | −2.03 | 2.96E−02 |
| | | | | PTNYSIKYNEEKRHVD | | |
| 56. | APC | APC_192 | 1146 | MYSEEEQHEEEERPTNYSIKYNEEK | −1.92 | 7.42E−02 |
| | | | | RHVDQPIDYSLKYATD | | |
| 57. | APC | APC_233 | 1392 | MLDSFESRSIASSVQSEPCSGMVSG | −2.46 | 1.30E−02 |
| | | | | IISPSDLPDSPGQTMP | | |
| 58. | APC | APC_234 | 1398 | MRSIASSVQSEPCSGMVSGIISPSD | −3.70 | 1.97E−03 |
| | | | | LPDSPGQTMPPSRSKT | | |
| 59. | APC | APC_236 | 1410 | MSGMVSGIISPSDLPDSPGQTMPPS | −2.54 | 1.42E−02 |
| | | | | RSKTPPPPPQTAQTKR | | |
| 60. | APC | APC_237 | 1416 | MIISPSDLPDSPGQTMPPSRSKTPP | −2.30 | 2.02E−02 |
| | | | | PPPQTAQTKREVPKNK | | |
| 61. | APC | APC_238 | 1422 | MLPDSPGQTMPPSRSKTPPPPPQTA | −2.21 | 2.29E−02 |
| | | | | QTKREVPKNKAPTAEK | | |
| 62. | APC | APC_257 | 1536 | MTESEQPKESNENQEKEAEKTIDSE | −3.26 | 4.44E−03 |
| | | | | KDLLDDSDDDDIEILE | | |
| 63. | APC | APC_258 | 1542 | MKESNENQEKEAEKTIDSEKDLLDD | −2.99 | 1.01E−02 |
| | | | | SDDDDIEILEECIISA | | |
| 64. | APC | APC_259 | 1548 | MQEKEAEKTIDSEKDLLDDSDDDDI | −2.03 | 6.27E−02 |
| | | | | EILEECIISAMPTKSS | | |
| 65. | APC | APC_260 | 1554 | MKTIDSEKDLLDDSDDDDIEILEEC | −3.18 | 4.04E−03 |
| | | | | IISAMPTKSSRKAKKP | | |
| 66. | APC | APC_266 | 1590 | MAKKPAQTASKLPPPVARKPSQLPV | −1.83 | 6.83E−02 |
| | | | | YKLLPSQNRLQPQKHV | | |
| 67. | APC | APC_268 | 1602 | MPPVARKPSQLPVYKLLPSQNRLQP | −1.79 | 5.62E−02 |
| | | | | QKHVSFTPGDDMPRVY | | |
| 68. | APC | APC_308 | 1842 | MPHHYTPIEGTPYCFSRNDSLSSLD | −2.84 | 8.93E−03 |
| | | | | FDDDDVDLSREKAELR | | |
| 69. | APC | APC_309 | 1848 | MIEGTPYCFSRNDSLSSLDFDDDDV | −2.79 | 9.63E−03 |
| | | | | DLSREKAELRKAKENK | | |
| 70. | APC | APC_397 | 2376 | MNLTKQTGLSKNASSIPRSESASKG | −2.12 | 4.28E−02 |
| | | | | LNQMNNGNGANKKVEL | | |
| 71. | APC | APC_398 | 2382 | MGLSKNASSIPRSESASKGLNQMNN | −1.87 | 7.66E−02 |
| | | | | GNGANKKVELSRMSST | | |
| 72. | APC | APC_400 | 2394 | MESASKGLNQMNNGNGANKKVELSR | −2.53 | 2.86E−02 |
| | | | | MSSTKSSGSESDRSER | | |
| 73. | APC | APC_402 | 2406 | MGNGANKKVELSRMSSTKSSGSESD | −1.80 | 4.08E−02 |
| | | | | RSERPVLVRQSTFIKE | | |
| 74. | APOB | APOB_358 | 2142 | MTALTKKYRITENDIQIALDDAKIN | −3.17 | 4.12E−03 |
| | | | | FNEKLSQLQTYMIQFD | | |
| 75. | APOB | APOB_360 | 2154 | MDIQIALDDAKINFNEKLSQLQTYM | −2.98 | 5.71E−03 |
| | | | | IQFDQYIKDSYDLHDL | | |
| 76. | APOB | APOB_361 | 2160 | MDDAKINFNEKLSQLQTYMIQFDQY | −2.91 | 6.36E−03 |
| | | | | IKDSYDLHDLKIAIAN | | |
| 77. | APOB | APOB_370 | 2214 | MHYHIRVNLVKTIHDLHLFIENIDF | −2.21 | 4.73E−02 |
| | | | | NKSGSSTASWIQNVDT | | |
| 78. | APOB | APOB_371 | 2220 | MNLVKTIHDLHLFIENIDFNKSGSS | −2.11 | 5.65E−02 |
| | | | | TASWIQNVDTKYQIRI | | |
| 79. | APOB | APOB_397 | 2376 | MLSNVLQQVKIKDYFEKLVGFIDDA | −2.28 | 3.16E−02 |
| | | | | VKKLNELSFKTFIEDV | | |
| 80. | APOB | APOB_398 | 2382 | MQVKIKDYFEKLVGFIDDAVKKLNE | −2.18 | 1.99E−02 |
| | | | | LSFKTFIEDVNKFLDM | | |
| 81. | APOB | APOB_399 | 2388 | MYFEKLVGFIDDAVKKLNELSFKTF | −2.56 | 1.38E−02 |
| | | | | IEDVNKFLDMLIKKLK | | |
| 82. | APOB | APOB_725 | 4344 | MFKLLKENLCLNLHKFNEFIQNELQ | −2.93 | 1.28E−02 |
| | | | | EASQELQQIHQYIMAL | | |
| 83. | APOB | APOB_726 | 4350 | MNLCLNLHKFNEFIQNELQEASQEL | −2.91 | 1.31E−02 |
| | | | | QQIHQYIMALREEYFD | | |
| 84. | AR | AR_105 | 624 | MKLKKLGNLKLQEEGEASSTTSPTE | −2.28 | 2.81E−02 |
| | | | | ETTQKLTVSHIEGYEC | | |
| 85. | AR | AR_106 | 630 | MNLKLQEEGEASSTTSPTEETTQKL | −1.88 | 5.07E−02 |
| | | | | TVSHIEGYECQPIFLN | | |
| 86. | ARHGAP26 | ARHGAP26_46 | 270 | MYLYVQEKRHFGTSWVKHYCTYQRD | −4.07 | 9.26E−04 |
| | | | | SKQITMVPFDQKSGGK | | |
| 87. | ARHGAP26 | ARHGAP26_48 | 282 | MSWVKHYCTYQRDSKQITMVPFDQK | −2.07 | 4.96E−02 |
| | | | | SGGKGGEDESVILKSC | | |
| 88. | ARHGAP26 | ARHGAP26_50 | 294 | MSKQITMVPFDQKSGGKGGEDESVI | −1.92 | 3.47E−02 |
| | | | | LKSCTRRKTDSIEKRF | | |
| 89. | ARHGAP35 | ARHGAP35_36 | 210 | MRDAHTFALSKKNLQVVETSARSNV | −2.27 | 4.08E−02 |
| | | | | NVDLAFSTLVQLIDKS | | |
| 90. | ARHGAP35 | ARHGAP35_37 | 216 | MALSKKNLQVVETSARSNVNVDLAF | −2.86 | 1.61E−02 |
| | | | | STLVQLIDKSRGKTKI | | |
| 91. | ARHGAP35 | ARHGAP35_76 | 450 | MPWEEARSFIMNEDFYQWLEESVYM | −3.11 | 4.58E−03 |
| | | | | DIYGKHQKQIIDKAKE | | |
| 92. | ARHGAP35 | ARHGAP35_77 | 456 | MSFIMNEDFYQWLEESVYMDIYGKH | −3.37 | 3.62E−03 |
| | | | | QKQIIDKAKEEFQELL | | |
| 93. | ARHGAP35 | ARHGAP35_78 | 462 | MDFYQWLEESVYMDIYGKHQKQIID | −3.32 | 3.98E−03 |
| | | | | KAKEEFQELLLEYSEL | | |
| 94. | ARHGAP35 | ARHGAP35_79 | 468 | MEESVYMDIYGKHQKQIIDKAKEEF | −3.32 | 3.98E−03 |
| | | | | QELLLEYSELFYELEL | | |
| 95. | ARHGAP35 | ARHGAP35_226 | 1350 | MEQKLHALKEVLKKFPKENHEVFKY | −1.92 | 6.59E−02 |
| | | | | VISHLNKVSHNNKVNL | | |
| 96. | ARHGAP35 | ARHGAP35_227 | 1356 | MLKEVLKKFPKENHEVFKYVISHLN | −1.91 | 6.72E−02 |
| | | | | KVSHNNKVNLMTSENL | | |
| 97. | ARHGEF12 | ARHGEF12_70 | 414 | MSKETRRIFLEFHQFFLDRSAHLKV | −2.13 | 5.40E−02 |
| | | | | SVPDEMSADLEKRRPE | | |
| 98. | ARHGEF12 | ARHGEF12_72 | 426 | MQFFLDRSAHLKVSVPDEMSADLEK | −1.95 | 7.12E−02 |
| | | | | RRPELIPEDLHRHYIQ | | |
| 99. | ARHGEF12 | ARHGEF12_74 | 438 | MSVPDEMSADLEKRRPELIPEDLHR | −2.80 | 9.45E−03 |
| | | | | HYIQTMQERVHPEVQR | | |
| 100. | ARHGEF12 | ARHGEF12_84 | 498 | MTKLDAERDKDRLTLEKERTCAEQI | −2.47 | 3.13E−02 |
| | | | | VAKIEEVLMTAQAVEE | | |
| 101. | ARHGEF12 | ARHGEF12_86 | 510 | MTLEKERTCAEQIVAKIEEVLMTAQ | −1.97 | 5.48E−02 |
| | | | | AVEEDKSSTMQYVILM | | |
| 102. | ARHGEF12 | ARHGEF12_87 | 516 | MTCAEQIVAKIEEVLMTAQAVEEDK | −1.89 | 5.84E−02 |
| | | | | SSTMQYVILMYMKHLG | | |
| 103. | ARHGEF12 | ARHGEF12_89 | 528 | MVLMTAQAVEEDKSSTMQYVILMYM | −1.87 | 6.19E−02 |
| | | | | KHLGVKVKEPRNLEHK | | |
| 104. | ARHGEF12 | ARHGEF12_155 | 924 | MQLKDIIPTQMQRLTKYPLLLDNIA | −1.83 | 3.92E−02 |
| | | | | KYTEWPTEREKVKKAA | | |
| 105. | ARHGEF12 | ARHGEF12_159 | 948 | MKYTEWPTEREKVKKAADHCRQILN | −3.14 | 5.42E−03 |
| | | | | YVNQAVKEAENKQRLE | | |
| 106. | ARHGEF12 | ARHGEF12_160 | 954 | MTEREKVKKAADHCRQILNYVNQAV | −2.98 | 5.72E−03 |
| | | | | KEAENKQRLEDYQRRL | | |
| 107. | ARHGEF12 | ARHGEF12_162 | 966 | MCRQILNYVNQAVKEAENKQRLEDY | −3.19 | 5.00E−03 |
| | | | | QRRLDTSSLKLSEYPN | | |
| 108. | ARHGEF12 | ARHGEF12_163 | 972 | MYVNQAVKEAENKQRLEDYQRRLDT | −2.74 | 1.04E−02 |
| | | | | SSLKLSEYPNVEELRN | | |
| 109. | ARHGEF12 | ARHGEF12_164 | 978 | MKEAENKQRLEDYQRRLDTSSLKLS | −2.89 | 8.32E−03 |
| | | | | EYPNVEELRNLDLTKR | | |
| 110. | ARHGEF12 | ARHGEF12_165 | 984 | MQRLEDYQRRLDTSSLKLSEYPNVE | −3.01 | 6.74E−03 |
| | | | | ELRNLDLTKRKMIHEG | | |
| 111. | ARHGEF12 | ARHGEF12_173 | 1032 | MDKTIDLYTLLLEDILVLLQKQDDR | −1.84 | 6.97E−02 |
| | | | | LVLRCHSKILASTADS | | |
| 112. | ARHGEF12 | ARHGEF12_176 | 1050 | MQKQDDRLVLRCHSKILASTADSKH | −4.04 | 9.80E−04 |
| | | | | TFSPVIKLSTVLVRQV | | |
| 113. | ARHGEF12 | ARHGEF12_180 | 1074 | MTFSPVIKLSTVLVRQVATDNKALF | −4.51 | 3.39E−04 |
| | | | | VISMSDNGAQIYELVA | | |
| 114. | ARHGEF12 | ARHGEF12_190 | 1134 | MKEQSTKPIPLPQSTPGEGDNDEED | −1.95 | 3.31E−02 |
| | | | | PSKLKEEQHGISVTGL | | |
| 115. | ARHGEF12 | ARHGEF12_198 | 1182 | MLESTLISSKPQSHSLSTSGKSEVR | −2.30 | 2.01E−02 |
| | | | | DLFVAERQFAKEQHTD | | |
| 116. | ARHGEF12 | ARHGEF12_201 | 1200 | MGKSEVRDLFVAERQFAKEQHTDGT | −4.03 | 1.01E−03 |
| | | | | LKEVGEDYQIAIPDSH | | |
| 117. | ARID1A | ARID1A335 | 2004 | MPGLLLILGKLILLHHKHPERKQAP | −2.89 | 8.10E−03 |
| | | | | LTYEKEEEQDQGVSCN | | |
| 118. | ARID1A | ARID1A339 | 2028 | MLTYEKEEEQDQGVSCNKVEWWWDC | −2.00 | 1.91E−02 |
| | | | | LEMLRENTLVTLANIS | | |
| 119. | ARID1A | ARID1A357 | 2136 | MATPPFSRLEKLYSTMVRFLSDRKN | −2.35 | 1.71E−02 |
| | | | | PVCREMAVVLLANLAQ | | |
| 120. | ARID1A | ARID1A359 | 2148 | MSTMVRFLSDRKNPVCREMAVVLLA | −1.94 | 1.09E−02 |
| | | | | NLAQGDSLAARAIAVQ | | |
| 121. | ARID2 | ARID2_65 | 384 | MEDNGVLICEYVDQDSYREIICHLT | −1.77 | 8.39E−02 |
| | | | | LPDVLLVISTLEVLYM | | |
| 122. | ARID2 | ARID2_66 | 390 | MICEYVDQDSYREIICHLTLPDVLL | −2.31 | 4.01E−02 |
| | | | | VISTLEVLYMLTEMGD | | |
| 123. | ARID2 | ARID2_89 | 528 | MNAHFEVNPDCSVSRAEMYSEYLST | −2.95 | 1.34E−02 |
| | | | | CSKLARGGILTSTGFY | | |
| 124. | ARID2 | ARID2_203 | 1212 | MPVQTLPATQASPAGQSSCTTATPP | −4.25 | 6.22E−04 |
| | | | | FKGDKIICQKEEEAKE | | |
| 125. | ARID2 | ARID2_206 | 1230 | MTTATPPFKGDKIICQKEEEAKEAT | −2.47 | 1.58E−02 |
| | | | | GLHVHERKIEVMENPS | | |
| 126. | ARID2 | ARID2_207 | 1236 | MFKGDKIICQKEEEAKEATGLHVHE | −2.48 | 1.55E−02 |
| | | | | RKIEVMENPSCRRGAT | | |
| 127. | ARID2 | ARID2_280 | 1674 | MFQRQRFSFITHLQDKHCSKDALLA | −2.84 | 1.38E−02 |
| | | | | GLKQDEPGQAGSQKSS | | |
| 128. | ARID2 | ARID2_281 | 1680 | MSFITHLQDKHCSKDALLAGLKQDE | −4.21 | 6.81E−04 |
| | | | | PGQAGSQKSSTKQPTV | | |
| 129. | ARID2 | ARID2_292 | 1746 | MGSRNLVFRDFTDEKEGPITKHIRL | −1.97 | 4.01E−02 |
| | | | | TAALILKNIGKYSECG | | |
| 130. | ARID2 | ARID2_293 | 1752 | MFRDFTDEKEGPITKHIRLTAALIL | −1.99 | 3.92E−02 |
| | | | | KNIGKYSECGRRLLKR | | |
| 131. | ARID2 | ARID2_294 | 1758 | MEKEGPITKHIRLTAALILKNIGKY | −2.05 | 2.37E−02 |
| | | | | SECGRRLLKRHENNLS | | |
| 132. | ARID2 | ARID2_295 | 1764 | MTKHIRLTAALILKNIGKYSECGRR | −2.27 | 2.12E−02 |
| | | | | LLKRHENNLSVLAISN | | |
| 133. | ARID3B | ARID3B12 | 66 | MGPLARVPPTAAVAQVFERGNMNSE | −4.34 | 5.06E−04 |
| | | | | PEEEDGGLEDEDGDDE | | |
| 134. | ARID3B | ARID3B13 | 72 | MPPTAAVAQVFERGNMNSEPEEEDG | −3.64 | 2.32E−03 |
| | | | | GLEDEDGDDEVAEVAE | | |
| 135. | ARID5B | ARID5B175 | 1044 | MKASEQESEGSKAAHGGHSGGGSEG | −1.84 | 4.08E−03 |
| | | | | HKLPLSSPIFPGLYSG | | |
| 136. | ARID5B | ARID5B179 | 1068 | MHKLPLSSPIFPGLYSGSLCNSGLN | −2.20 | 4.19E−02 |
| | | | | SRLPAGYSHSLQYLKN | | |
| 137. | ASXL1 | ASXL1_13 | 72 | MKLPGRISLFTLKKDALQWSRHPAT | −3.87 | 6.08E−04 |
| | | | | VEGEEPEDTADVESCG | | |
| 138. | ASXL1 | ASXL1_18 | 102 | MEDTADVESCGSNEASTVSGENDVS | −3.15 | 5.31E−03 |
| | | | | LDETSSNASCSTESQS | | |
| 139. | ASXL1 | ASXL1_61 | 360 | MQKLGLTKEESLQQNVGQEEAEIKS | −3.39 | 3.51E−03 |
| | | | | GLCVPGESVRIQRGPA | | |
| 140. | ASXL1 | ASXL1_62 | 366 | MKEESLQQNVGQEEAEIKSGLCVPG | −2.29 | 3.05E−02 |
| | | | | ESVRIQRGPATRQRDG | | |
| 141. | ASXL1 | ASXL1_70 | 414 | MDLRTRARRNLYKKQESEQAGVAKD | −3.47 | 3.04E−03 |
| | | | | AKSVASDVPLYKDGEA | | |
| 142. | ASXL1 | ASXL1_146 | 870 | MSCPPMRESDTRQENLKTKALVSNS | −2.08 | 5.55E−02 |
| | | | | SLHWIPIPSNDEVVKQ | | |
| 143. | ASXL1 | ASXL1_147 | 876 | MESDTRQENLKTKALVSNSSLHWIP | −1.75 | 9.00E−02 |
| | | | | IPSNDEVVKQPKPESR | | |
| 144. | ASXL2 | ASXL2_105 | 624 | MHSVNPEDRFCLSSPTEALKMGYTD | −3.19 | 5.01E−03 |
| | | | | CKNATGESSSSKEDDT | | |
| 145. | ASXL2 | ASXL2_106 | 630 | MDRFCLSSPTEALKMGYTDCKNATG | −3.32 | 3.98E−03 |
| | | | | ESSSSKEDDTDEESTG | | |
| 146. | ASXL2 | ASXL2_107 | 636 | MSPTEALKMGYTDCKNATGESSSSK | −3.34 | 3.87E−03 |
| | | | | EDDTDEESTGDEQESV | | |
| 147. | ATF7IP | ATF7IP_78 | 462 | MTNPDLEEKMESSFGSPSKQESSES | −2.50 | 2.20E−02 |
| | | | | LPKEAFLVLSDEEDIS | | |
| 148. | ATF7IP | ATF7IP_79 | 468 | MEKMESSFGSPSKQESSESLPKEAF | −2.01 | 5.06E−02 |
| | | | | LVLSDEEDISGEKDES | | |
| 149. | ATF7IP | ATF7IP_80 | 474 | MFGSPSKQESSESLPKEAFLVLSDE | −3.79 | 1.67E−03 |
| | | | | EDISGEKDESEVISQN | | |
| 150. | ATF7IP | ATF7IP_98 | 582 | MKITAKGDINQKLQKVIQWLLEEKL | −2.30 | 4.07E−02 |
| | | | | CALQCAVFDKTLAELK | | |
| 151. | ATF7IP | ATF7IP_99 | 588 | MDINQKLQKVIQWLLEEKLCALQCA | −2.72 | 1.60E−02 |
| | | | | VFDKTLAELKTRVEKI | | |
| 152. | ATF7IP | ATF7IP_101 | 600 | MLLEEKLCALQCAVFDKTLAELKTR | −2.95 | 7.48E−03 |
| | | | | VEKIECNKRHKTVLTE | | |
| 153. | ATR | ATR_44 | 258 | MGGLPAQPASTFFSSFLELLKHLVE | −1.99 | 5.33E−02 |
| | | | | MDTDQLKLYEEPLSKL | | |
| 154. | ATR | ATR_45 | 264 | MPASTFFSSFLELLKHLVEMDTDQL | −1.85 | 6.44E−02 |
| | | | | KLYEEPLSKLIKTLFP | | |
| 155. | ATR | ATR_46 | 270 | MSSFLELLKHLVEMDTDQLKLYEEP | −2.09 | 4.65E−02 |
| | | | | LSKLIKTLFPFEAEAY | | |
| 156. | ATR | ATR_59 | 348 | MLLQYFLKFVPAGYESALQVRKVYV | −2.26 | 4.24E−02 |
| | | | | RNICKALLDVLGIEVD | | |
| 157. | ATR | ATR_60 | 354 | MKFVPAGYESALQVRKVYVRNICKA | −1.81 | 8.08E−02 |
| | | | | LLDVLGIEVDAEYLLG | | |
| 158. | ATR | ATR_164 | 978 | MANVFDFPDLNRFLTRTLQVLLPDL | −2.48 | 2.33E−02 |
| | | | | AAKASPAASALIRTLG | | |
| 159. | ATR | ATR_165 | 984 | MPDLNRFLTRTLQVLLPDLAAKASP | −2.31 | 2.84E−02 |
| | | | | AASALIRTLGKQLNVN | | |
| 160. | ATR | ATR_166 | 990 | MLTRTLQVLLPDLAAKASPAASALI | −2.41 | 2.45E−02 |
| | | | | RTLGKQLNVNRREILI | | |
| 161. | ATR | ATR_167 | 996 | MVLLPDLAAKASPAASALIRTLGKQ | −2.49 | 2.17E−02 |
| | | | | LNVNRREILINNFKYI | | |
| 162. | ATR | ATR_233 | 1392 | MAYGLLMELTRAYLAYADNSRAQDS | −2.58 | 2.62E−02 |
| | | | | AAYAIQELLSIYDCRE | | |
| 163. | ATR | ATR_235 | 1404 | MLAYADNSRAQDSAAYAIQELLSIY | −1.76 | 8.44E−02 |
| | | | | DCREMETNGPGHQLWR | | |
| 164. | ATR | ATR_301 | 1800 | MSVRLGQLLLSAKKRDITAFYDSLK | −2.47 | 2.99E−02 |
| | | | | LVRAEQIVPLSAASFE | | |
| 165. | ATR | ATR_302 | 1806 | MLLLSAKKRDITAFYDSLKLVRAEQ | −3.60 | 2.38E−03 |
| | | | | IVPLSAASFERGSYQR | | |
| 166. | ATR | ATR_306 | 1830 | MIVPLSAASFERGSYQRGYEYIVRL | −1.77 | 8.51E−02 |
| | | | | HMLCELEHSIKPLFQH | | |
| 167. | ATR | ATR_309 | 1848 | MEYIVRLHMLCELEHSIKPLFQHSP | −2.22 | 2.26E−02 |
| | | | | GDSSQEDSLNWVARLE | | |
| 168. | ATRX | ATRX_133 | 792 | MDFKKKVIKMEQQYESSSDGTEKLP | −2.99 | 7.02E−03 |
| | | | | EREEICHFPKGIKQIK | | |
| 169. | ATRX | ATRX_134 | 798 | MIKMEQQYESSSDGTEKLPEREEIC | −2.97 | 7.21E−03 |
| | | | | HFPKGIKQIKNGTTDG | | |
| 170. | ATRX | ATRX_135 | 804 | MYESSSDGTEKLPEREEICHFPKGI | −2.94 | 7.58E−03 |
| | | | | KQIKNGTTDGEKKSKK | | |
| 171. | ATRX | ATRX_247 | 1476 | MIGYEMYRNLAQGRNVKSRKLKEIF | −2.21 | 1.88E−02 |
| | | | | NKALVDPGPDFVVCDE | | |
| 172. | ATRX | ATRX_248 | 1482 | MRNLAQGRNVKSRKLKEIFNKALVD | −2.17 | 2.43E−02 |
| | | | | PGPDFVVCDEGHILKN | | |
| 173. | ATRX | ATRX_249 | 1488 | MRNVKSRKLKEIFNKALVDPGPDFV | −2.23 | 2.79E−02 |
| | | | | VCDEGHILKNEASAVS | | |
| 174. | ATRX | ATRX_278 | 1662 | MKAGAKLFQDFQMLSRIWTHPWCLQ | −2.87 | 8.47E−03 |
| | | | | LDYISKENKGYFDEDS | | |
| 175. | ATRX | ATRX_282 | 1686 | MLDYISKENKGYFDEDSMDEFIASD | −2.19 | 4.95E−02 |
| | | | | SDETSMSLSSDDYTKK | | |
| 176. | ATRX | ATRX_286 | 1710 | MSDETSMSLSSDDYTKKKKKGKKGK | −1.79 | 4.11E−02 |
| | | | | KDSSSSGSGSDNDVEV | | |
| 177. | ATRX | ATRX_302 | 1806 | MAEVLEHSGKMVLLFEILRMAEEIG | −2.10 | 5.42E−02 |
| | | | | DKVLVFSQSLISLDLI | | |
| 178. | ATRX | ATRX_303 | 1812 | MSGKMVLLFEILRMAEEIGDKVLVF | −2.43 | 3.35E−02 |
| | | | | SQSLISLDLIEDFLEL | | |
| 179. | ATRX | ATRX_323 | 1932 | MASWNPSYDIQSIFRVYRFGQTKPV | −3.06 | 6.26E−03 |
| | | | | YVYRFLAQGTMEDKIY | | |
| 180. | ATRX | ATRX_324 | 1938 | MYDIQSIFRVYRFGQTKPVYVYRFL | −2.85 | 8.83E−03 |
| | | | | AQGTMEDKIYDRQVTK | | |
| 181. | ATRX | ATRX_331 | 1980 | MLSFRVVDQQQVERHFTMNELTELY | −2.88 | 8.35E−03 |
| | | | | TFEPDLLDDPNSEKKK | | |
| 182. | ATXN3 | ATXN3_16 | 90 | MLILFNSPEYQRLRIDPINERSFIC | −2.21 | 2.30E−02 |
| | | | | NYKEHWFTVRKLGKQW | | |
| 183. | ATXN3 | ATXN3_18 | 102 | MRIDPINERSFICNYKEHWFTVRKL | −2.45 | 1.61E−02 |
| | | | | GKQWFNLNSLLTGPEL | | |
| 184. | ATXN3 | ATXN3_19 | 108 | MERSFICNYKEHWFTVRKLGKQWFN | −2.54 | 1.42E−02 |
| | | | | LNSLLTGPELISDTYL | | |
| 185. | ATXN3 | ATXN3_20 | 114 | MNYKEHWFTVRKLGKQWFNLNSLLT | −2.72 | 8.70E−03 |
| | | | | GPELISDTYLALFLAQ | | |
| 186. | ATXN3 | ATXN3_21 | 120 | MFTVRKLGKQWFNLNSLLTGPELIS | −2.42 | 3.22E−02 |
| | | | | DTYLALFLAQLQQEGY | | |
| 187. | AURKA | AURKA_20 | 114 | MASKQKNEESKKRQWALEDFEIGRP | −1.86 | 8.02E−02 |
| | | | | LGKGKFGNVYLAREKQ | | |
| 188. | AURKA | AURKA_21 | 120 | MEESKKRQWALEDFEIGRPLGKGKF | −1.87 | 7.78E−02 |
| | | | | GNVYLAREKQSKFILA | | |
| 189. | AURKA | AURKA_33 | 192 | MILRLYGYFHDATRVYLILEYAPLG | −3.12 | 8.89E−03 |
| | | | | TVYRELQKLSKFDEQR | | |
| 190. | AURKA | AURKA_34 | 198 | MYFHDATRVYLILEYAPLGTVYREL | −2.82 | 1.39E−02 |
| | | | | QKLSKFDEQRTATYIT | | |
| 191. | AURKA | AURKA_37 | 216 | MTVYRELQKLSKFDEQRTATYITEL | −2.37 | 3.67E−02 |
| | | | | ANALSYCHSKRVIHRD | | |
| 192. | AURKA | AURKA_38 | 222 | MQKLSKFDEQRTATYITELANALSY | −2.45 | 3.23E−02 |
| | | | | CHSKRVIHRDIKPENL | | |
| 193. | AXIN1 | AXIN1_4 | 18 | MDAPRPPVPGEEGELVSTDPRPASY | −1.87 | 3.08E−02 |
| | | | | SFCSGKGVGIKGETST | | |
| 194. | AXIN1 | AXIN1_5 | 24 | MVPGEEGELVSTDPRPASYSFCSGK | −3.74 | 1.84E−03 |
| | | | | GVGIKGETSTATPRRS | | |
| 195. | AXIN1 | AXIN1_6 | 30 | MELVSTDPRPASYSFCSGKGVGIKG | −2.29 | 2.05E−02 |
| | | | | ETSTATPRRSDLDLGY | | |
| 196. | AXIN1 | AXIN1_45 | 264 | MGRLPQKLLLETAAPRVSSSRRYSE | −2.14 | 2.54E−02 |
| | | | | GREFRYGSWREPVNPY | | |
| 197. | AXIN1 | AXIN1_46 | 270 | MLLLETAAPRVSSSRRYSEGREFRY | −1.94 | 6.62E−02 |
| | | | | GSWREPVNPYYVNAGY | | |
| 198. | AXIN1 | AXIN1_47 | 276 | MAPRVSSSRRYSEGREFRYGSWREP | −2.03 | 3.25E−02 |
| | | | | VNPYYVNAGYALAPAT | | |
| 199. | AXIN1 | AXIN1_56 | 330 | MDTLSLTDSSVDGIPPYRIRKQHRR | −2.35 | 2.11E−03 |
| | | | | EMQESVQVNGRVPLPH | | |
| 200. | AXIN1 | AXIN1_58 | 342 | MIPPYRIRKQHRREMQESVQVNGRV | −2.00 | 1.37E−03 |
| | | | | PLPHIPRTYRVPKEVR | | |
| 201. | BABAM2 | BABAM252 | 306 | MCFLVHIDLPLFFPRDQPTLTFQSV | −2.23 | 1.71E−02 |
| | | | | YHFTNSGQLYSQAQKN | | |
| 202. | BABAM2 | BABAM2_53 | 312 | MDLPLFFPRDQPTLTFQSVYHFTNS | −3.96 | 1.31E−03 |
| | | | | GQLYSQAQKNYPYSPR | | |
| 203. | BABAM2 | BABAM255 | 324 | MLTFQSVYHFTNSGQLYSQAQKNYP | −1.86 | 5.05E−02 |
| | | | | YSPRWDGNEMAKRAKA | | |
| 204. | BANP | BANP_54 | 318 | MMSTPPPASELPQPQPQPQALHYAL | −3.75 | 4.49E−04 |
| | | | | ANAQQVQIHQIGEDGQ | | |
| 205. | BANP | BANP_58 | 342 | MANAQQVQIHQIGEDGQVQVIPQGH | −3.56 | 2.59E−03 |
| | | | | LHIAQVPQGEQVQITQ | | |
| 206. | BANP | BANP_59 | 348 | MQIHQIGEDGQVQVIPQGHLHIAQV | −4.78 | 1.73E−04 |
| | | | | PQGEQVQITQDSEGNL | | |
| 207. | BANP | BANP_60 | 354 | MEDGQVQVIPQGHLHIAQVPQGEQV | −1.78 | 3.09E−04 |
| | | | | QITQDSEGNLQIHHVG | | |
| 208. | BAP1 | BAP1_18 | 102 | MCSSVDLGPTLSRMKDFTKGFSPES | −2.55 | 1.90E−02 |
| | | | | KGYAIGNAPELAKAHN | | |
| 209. | BAP1 | BAP1_19 | 108 | MGPTLSRMKDFTKGFSPESKGYAIG | −3.30 | 4.15E−03 |
| | | | | NAPELAKAHNSHARPE | | |
| 210. | BAP1 | BAP1_30 | 174 | MPITGRLFELDGLKVYPIDHGPWGE | −3.57 | 2.55E−03 |
| | | | | DEEWTDKARRVIMERI | | |
| 211. | BAP1 | BAP1_32 | 186 | MKVYPIDHGPWGEDEEWTDKARRVI | −2.98 | 7.13E−03 |
| | | | | MERIGLATAGEPYHDI | | |
| 212. | BAP1 | BAP1_34 | 198 | MDEEWTDKARRVIMERIGLATAGEP | −2.53 | 1.54E−03 |
| | | | | YHDIRFNLMAVVPDRR | | |
| 213. | BAP1 | BAP1_35 | 204 | MKARRVIMERIGLATAGEPYHDIRF | −2.03 | 8.74E−03 |
| | | | | NLMAVVPDRRIKYEAR | | |
| 214. | BAP1 | BAP1_65 | 384 | MRVPVRPPQQYSDDEDDYEDDEEDD | −3.90 | 1.33E−03 |
| | | | | VQNTNSALRYKGKGTG | | |
| 215. | BAP1 | BAP1_68 | 402 | MDDEEDDVQNTNSALRYKGKGTGKP | −1.83 | 1.84E−02 |
| | | | | GALSGSADGQLSVLQP | | |
| 216. | BARD1 | BARD1_42 | 246 | MSVISSPQINGEIDLLASGSLTESE | −3.32 | 3.96E−03 |
| | | | | CFGSLTEVSLPLAEQI | | |
| 217. | BARD1 | BARD1_44 | 258 | MDLLASGSLTESECFGSLTEVSLPL | −2.28 | 4.31E−02 |
| | | | | AEQIESPDTKSRNEVV | | |
| 218. | BARD1 | BARD1_102 | 606 | MVVVPGDAVQSTLKCMLGILNGCWI | −1.77 | 4.25E−02 |
| | | | | LKFEWVKACLRRKVCE | | |
| 219. | BCAS3 | BCAS3_50 | 294 | MPSGVTEDDVAIHSNSRRSPLVPGI | −2.27 | 1.28E−02 |
| | | | | ITVIDTETVGEGQVLV | | |
| 220. | BCAS3 | BCAS3_53 | 312 | MPLVPGIITVIDTETVGEGQVLVSE | −3.34 | 5.35E−03 |
| | | | | DSDSDGIVAHFPAHEK | | |
| 221. | BCAS3 | BCAS3_142 | 846 | MHMSSMEHTEEGLRERLADAMAESP | −2.67 | 1.35E−02 |
| | | | | SRDVVGSGTELQREGS | | |
| 222. | BCAS3 | BCAS3_143 | 852 | MHTEEGLRERLADAMAESPSRDVVG | −2.81 | 1.07E−02 |
| | | | | SGTELQREGSIETLSN | | |
| 223. | BCL10 | BCL10_8 | 42 | MAKKILSREDTEEISCRTSSRKRAG | −1.96 | 6.93E−02 |
| | | | | KLLDYLQENPKGLDTL | | |
| 224. | BCL10 | BCL10_9 | 48 | MREDTEEISCRTSSRKRAGKLLDYL | −1.99 | 6.39E−02 |
| | | | | QENPKGLDTLVESIRR | | |
| 225. | BCL10 | BCL10_10 | 54 | MISCRTSSRKRAGKLLDYLQENPKG | −1.80 | 8.20E−02 |
| | | | | LDTLVESIRREKTQNF | | |
| 226. | BCL10 | BCL10_11 | 60 | MSRKRAGKLLDYLQENPKGLDTLVE | −1.90 | 7.26E−02 |
| | | | | SIRREKTQNFLIQKIT | | |
| 227. | BCL9 | BCL9_4 | 18 | MSPKSKQEVMVRPPTVMSPSGNPQL | −2.96 | 1.13E−02 |
| | | | | DSKFSNQGKQGGSASQ | | |
| 228. | BCL9 | BCL9_6 | 30 | MPTVMSPSGNPQLDSKFSNQGKQGG | −2.34 | 1.70E−02 |
| | | | | SASQSQPSPCDSKSGG | | |
| 229. | BCL9 | BCL9_29 | 168 | MAQKTPAKVVYVFSTEMANKAAEAV | −2.32 | 4.06E−02 |
| | | | | LKGQVETIVSFHIQNI | | |
| 230. | BCL9 | BCL9_30 | 174 | MKVVYVFSTEMANKAAEAVLKGQVE | −2.17 | 5.04E−02 |
| | | | | TIVSFHIQNISNNKTE | | |
| 231. | BCL9 | BCL9_66 | 390 | MNPGVLDGPQKKPEGPIQAMMAQSQ | −2.04 | 2.90E−02 |
| | | | | SLGKGPGPRTDVGAPF | | |
| 232. | BCL9 | BCL9_67 | 396 | MGPQKKPEGPIQAMMAQSQSLGKGP | −2.14 | 2.09E−02 |
| | | | | GPRTDVGAPFGPQGHR | | |
| 233. | BCL9 | BCL9_74 | 438 | MPFSPDEMVPPSMNSQSGTIGPDHL | −3.01 | 6.73E−03 |
| | | | | DHMTPEQIAWLKLQQE | | |
| 234. | BCL9 | BCL9_114 | 678 | MRIPVEGPLSPSRGDFPKGIPPQMG | −2.72 | 1.79E−02 |
| | | | | PGRELEFGMVPSGMKG | | |
| 235. | BCL9 | BCL9_116 | 690 | MGDFPKGIPPQMGPGRELEFGMVPS | −2.61 | 2.50E−02 |
| | | | | GMKGDVNLNVNMGSNS | | |
| 236. | BCL9 | BCL9_118 | 702 | MPGRELEFGMVPSGMKGDVNLNVNM | −2.69 | 1.62E−02 |
| | | | | GSNSQMIPQKMREAGA | | |
| 237. | BCOR | BCOR_122 | 726 | MPMLIPHTPIEITKEEKPERRSRSH | −1.79 | 8.84E−02 |
| | | | | ERARYEDPTLRNRFSE | | |
| 238. | BCOR | BCOR_123 | 732 | MTPIEITKEEKPERRSRSHERARYE | −2.41 | 3.47E−02 |
| | | | | DPTLRNRFSEILETSS | | |
| 239. | BCOR | BCOR_133 | 792 | MQGKTVVKSDKLVYVDLLREEPDAK | −3.90 | 1.34E−03 |
| | | | | TDTNVSKPSFAAESVG | | |
| 240. | BCR | BCR_163 | 972 | MRILCYEKCYNKTKIPKEDGESTDR | −3.87 | 1.42E−03 |
| | | | | LMGKGQVQLDPQALQD | | |
| 241. | BCR | BCR_164 | 978 | MKCYNKTKIPKEDGESTDRLMGKGQ | −1.78 | 5.35E−02 |
| | | | | VQLDPQALQDRDWQRT | | |
| 242. | BCR | BCR_165 | 984 | MKIPKEDGESTDRLMGKGQVQLDPQ | −3.53 | 1.47E−03 |
| | | | | ALQDRDWQRTVIAMNG | | |
| 243. | BMI1 | BMI1_20 | 114 | MGEVADEDKRIITDDEIISLSIEFF | −3.19 | 3.94E−03 |
| | | | | DQNRLDRKVNKDKEKS | | |
| 244. | BMI1 | BMI1_21 | 120 | MDKRIITDDEIISLSIEFFDQNRLD | −3.30 | 4.11E−03 |
| | | | | RKVNKDKEKSKEEVND | | |
| 245. | BMI1 | BMI1_22 | 126 | MDDEIISLSIEFFDQNRLDRKVNKD | −3.27 | 4.39E−03 |
| | | | | KEKSKEEVNDKRYLRC | | |
| 246. | BRAF | BRAF_61 | 360 | MDRSSSAPNVHINTIEPVNIDDLIR | −2.25 | 2.48E−02 |
| | | | | DQGFRGDGGSTTGLSA | | |
| 247. | BRAF | BRAF_62 | 366 | MPNVHINTIEPVNIDDLIRDQGFRG | −1.79 | 2.72E−02 |
| | | | | DGGSTTGLSATPPASL | | |
| 248. | BRAF | BRAF_64 | 378 | MIDDLIRDQGFRGDGGSTTGLSATP | −2.28 | 2.64E−02 |
| | | | | PASLPGSLTNVKALQK | | |
| 249. | BRAF | BRAF_66 | 390 | MDGGSTTGLSATPPASLPGSLTNVK | −2.20 | 2.34E−02 |
| | | | | ALQKSPGPQRERKSSS | | |
| 250. | BRCA1 | BRCA1_63 | 372 | MCEQKEENQGKNESNIKPVQTVNIT | −2.24 | 2.20E−02 |
| | | | | AGFPVVGQKDKPVDNA | | |
| 251. | BRCA1 | BRCA1_65 | 384 | MSNIKPVQTVNITAGFPVVGQKDKP | −1.82 | 5.99E−02 |
| | | | | VDNAKCSIKGGSRFCL | | |
| 252. | BRCA1 | BRCA1_138 | 822 | MENNQEEQSMDSNLGEAASGCESET | −3.00 | 1.25E−02 |
| | | | | SVSEDCSGLSSQSDIL | | |
| 253. | BRCA1 | BRCA1_139 | 828 | MQSMDSNLGEAASGCESETSVSEDC | −2.55 | 1.39E−02 |
| | | | | SGLSSQSDILTTQQRD | | |
| 254. | BRCA2 | BRCA2_46 | 270 | MGNSFKVNSCKDHIGKSMPNVLEDE | −3.05 | 6.33E−03 |
| | | | | VYETVVDTSEEDSFSL | | |
| 255. | BRCA2 | BRCA2_51 | 300 | MDTSEEDSFSLCFSKCRTKNLQKVR | −1.88 | 3.65E−02 |
| | | | | TSKTRKKIFHEANADE | | |
| 256. | BRCA2 | BRCA2_66 | 390 | MLACEWSQLTLSGLNGAQMEKIPLL | −2.17 | 4.98E−02 |
| | | | | HISSCDQNISEKDLLD | | |
| 257. | BRCA2 | BRCA2_67 | 396 | MQLTLSGLNGAQMEKIPLLHISSCD | −2.90 | 8.16E−03 |
| | | | | QNISEKDLLDTENKRK | | |
| 258. | BRCA2 | BRCA2_68 | 402 | MLNGAQMEKIPLLHISSCDQNISEK | −3.09 | 5.94E−03 |
| | | | | DLLDTENKRKKDFLTS | | |
| 259. | BRCA2 | BRCA2_69 | 408 | MEKIPLLHISSCDQNISEKDLLDTE | −2.57 | 1.36E−02 |
| | | | | NKRKKDFLTSENSLPR | | |
| 260. | BRCA2 | BRCA2_122 | 726 | MSDIKEEVLAAACHPVQHSKVEYSD | −2.42 | 2.79E−02 |
| | | | | TDFQSQKSLLYDHENA | | |
| 261. | BRCA2 | BRCA2_124 | 738 | MHPVQHSKVEYSDTDFQSQKSLLYD | −1.90 | 7.74E−02 |
| | | | | HENASTLILTPTSKDV | | |
| 262. | BRCA2 | BRCA2_130 | 774 | MSKDVLSNLVMISRGKESYKMSDKL | −2.20 | 4.84E−02 |
| | | | | KGNNYESDVELTKNIP | | |
| 263. | BRCA2 | BRCA2_131 | 780 | MNLVMISRGKESYKMSDKLKGNNYE | −2.06 | 5.95E−02 |
| | | | | SDVELTKNIPMEKNQD | | |
| 264. | BRCA2 | BRCA2_132 | 786 | MRGKESYKMSDKLKGNNYESDVELT | −2.17 | 5.06E−02 |
| | | | | KNIPMEKNQDVCALNE | | |
| 265. | BRCA2 | BRCA2_143 | 852 | MQNTNLRVIQKNQEETTSISKITVN | −1.95 | 6.43E−02 |
| | | | | PDSEELFSDNENNFVF | | |
| 266. | BRCA2 | BRCA2_144 | 858 | MVIQKNQEETTSISKITVNPDSEEL | −1.83 | 7.86E−02 |
| | | | | FSDNENNFVFQVANER | | |
| 267. | BRCA2 | BRCA2_154 | 918 | MEPIFKNSTMVLYGDTGDKQATQVS | −3.21 | 4.83E−03 |
| | | | | IKKDLVYVLAEENKNS | | |
| 268. | BRCA2 | BRCA2_155 | 924 | MSTMVLYGDTGDKQATQVSIKKDLV | −3.25 | 4.52E−03 |
| | | | | YVLAEENKNSVKQHIK | | |
| 269. | BRCA2 | BRCA2_156 | 930 | MGDTGDKQATQVSIKKDLVYVLAEE | −3.17 | 5.15E−03 |
| | | | | NKNSVKQHIKMTLGQD | | |
| 270. | BRCA2 | BRCA2_226 | 1350 | MKDETDLLFTDQHNICLKLSGQFMK | −2.02 | 4.61E−02 |
| | | | | EGNTQIKEDLSDLTFL | | |
| 271. | BRCA2 | BRCA2_228 | 1362 | MNICLKLSGQFMKEGNTQIKEDLSD | −2.13 | 4.40E−02 |
| | | | | LTFLEVAKAQEACHGN | | |
| 272. | BRCA2 | BRCA2_277 | 1656 | MNQSPYSVIENSALAFYTSCSRKTS | −2.01 | 3.06E−02 |
| | | | | VSQTSLLEAKKWLREG | | |
| 273. | BRCA2 | BRCA2_284 | 1698 | MDGQPERINTADYVGNYLYENNSNS | −2.79 | 9.72E−03 |
| | | | | TIAENDKNHLSEKQDT | | |
| 274. | BRCA2 | BRCA2_285 | 1704 | MINTADYVGNYLYENNSNSTIAEND | −2.72 | 1.07E−02 |
| | | | | KNHLSEKQDTYLSNSS | | |
| 275. | BRCA2 | BRCA2_286 | 1710 | MVGNYLYENNSNSTIAENDKNHLSE | −2.80 | 9.57E−03 |
| | | | | KQDTYLSNSSMSNSYS | | |
| 276. | BRCA2 | BRCA2_405 | 2424 | MNINLEENRQKQNIDGHGSDDSKNK | −1.82 | 4.99E−02 |
| | | | | INDNEIHQFNKNNSNQ | | |
| 277. | BRCA2 | BRCA2_406 | 2430 | MNRQKQNIDGHGSDDSKNKINDNEI | −2.04 | 2.90E−02 |
| | | | | HQFNKNNSNQAAAVTF | | |
| 278. | BRCA2 | BRCA2_407 | 2436 | MIDGHGSDDSKNKINDNEIHQFNKN | −1.96 | 3.28E−02 |
| | | | | NSNQAAAVTFTKCEEE | | |
| 279. | BRCA2 | BRCA2_503 | 3012 | MTSKSKSKSERANIQLAATKKTQYQ | −2.18 | 5.04E−02 |
| | | | | QLPVSDEILFQIYQPR | | |
| 280. | BRCA2 | BRCA2_511 | 3060 | MLDPDFQPSCSEVDLIGFVVSVVKK | −1.80 | 6.39E−02 |
| | | | | TGLAPFVYLSDECYNL | | |
| 281. | BRCA2 | BRCA2_538 | 3222 | MEIYYQSPLSLCMAKRKSVSTPVSA | −3.42 | 3.34E−03 |
| | | | | QMTSKSCKGEKEIDDQ | | |
| 282. | BRCA2 | BRCA2_539 | 3228 | MPLSLCMAKRKSVSTPVSAQMTSKS | −3.56 | 2.59E−03 |
| | | | | CKGEKEIDDQKNCKKR | | |
| 283. | BRCC3 | BRCC3_33 | 192 | MSSSQHISIEGQKEEERYERIEIPI | −3.44 | 3.19E−03 |
| | | | | HIVPHVTIGKVCLESA | | |
| 284. | BRCC3 | BRCC3_35 | 204 | MEEERYERIEIPIHIVPHVTIGKVC | −2.43 | 9.10E−03 |
| | | | | LESAVELPKILCQEEQ | | |
| 285. | BRD7 | BRD7_8 | 42 | MGHDSSLFEDKNDHDKHKDRKRKKR | −2.49 | 1.52E−02 |
| | | | | KKGEKQIPGEEKGRKR | | |
| 286. | BRD7 | BRD7_9 | 48 | MFEDKNDHDKHKDRKRKKRKKGEKQ | −2.81 | 9.34E−03 |
| | | | | IPGEEKGRKRRRVKED | | |
| 287. | BRD7 | BRD7_66 | 390 | MDKRNKVTPVLYLNYGPYSSYAPHY | −1.99 | 6.34E−02 |
| | | | | DSTFANISKDDSDLIY | | |
| 288. | BRD7 | BRD7_67 | 396 | MTPVLYLNYGPYSSYAPHYDSTFAN | −1.94 | 7.00E−02 |
| | | | | ISKDDSDLIYSTYGED | | |
| 289. | BRMS1 | BRMS1_5 | 24 | MGEEEESEEERSGSQTESEEESSEM | −3.71 | 1.94E−03 |
| | | | | DDEDYERRRSECVSEM | | |
| 290. | BRMS1 | BRMS1_6 | 30 | MEEERSGSQTESEEESSEMDDEDYE | −3.55 | 2.66E−03 |
| | | | | RRRSECVSEMLDLEKQ | | |
| 291. | BRMS1 | BRMS1_11 | 60 | MVSEMLDLEKQFSELKEKLFRERLS | −2.51 | 2.98E−02 |
| | | | | QLRLRLEEVGAERAPE | | |
| 292. | BRMS1 | BRMS1_32 | 186 | MARGSSRSWDSLPPSKRKKAPLVSG | −2.06 | 1.99E−02 |
| | | | | PYIVYMLQEIDILEDW | | |
| 293. | BRMS1 | BRMS1_35 | 204 | MAPLVSGPYIVYMLQEIDILEDWTA | −2.23 | 2.25E−02 |
| | | | | IKKARAAVSPQKRKSD | | |
| 294. | BUB1B | BUB1B_23 | 132 | MNEPLDMYSYLHNQGIGVSLAQFYI | −1.91 | 5.00E−02 |
| | | | | SWAEEYEARENFRKAD | | |
| 295. | BUB1B | BUB1B_30 | 174 | MFQEGIQQKAEPLERLQSQHRQFQA | −1.94 | 4.60E−02 |
| | | | | RVSRQTLLALEKEEEE | | |
| 296. | BUB1B | BUB1B_31 | 180 | MQKAEPLERLQSQHRQFQARVSRQT | −1.96 | 4.14E−02 |
| | | | | LLALEKEEEEEVFESS | | |
| 297. | BUB1B | BUB1B_114 | 678 | MATHSSGFSGSSASVASTSSIKCLQ | −2.28 | 2.09E−02 |
| | | | | IPEKLELTNETSENPT | | |
| 298. | BUB1B | BUB1B_118 | 702 | MIPEKLELTNETSENPTQSPWCSQY | −2.11 | 5.16E−02 |
| | | | | RRQLLKSLPELSASAE | | |
| 299. | BUB1B | BUB1B_119 | 708 | MLTNETSENPTQSPWCSQYRRQLLK | −3.06 | 1.09E−02 |
| | | | | SLPELSASAELCIEDR | | |
| 300. | BUB1B | BUB1B_123 | 732 | MSLPELSASAELCIEDRPMPKLEIE | −3.21 | 4.82E−03 |
| | | | | KEIELGNEDYCIKREY | | |
| 301. | BUB1B | BUB1B_125 | 744 | MIEDRPMPKLEIEKEIELGNEDYCI | −2.70 | 2.09E−02 |
| | | | | KREYLICEDYKLFWVA | | |
| 302. | BUB1B | BUB1B_126 | 750 | MPKLEIEKEIELGNEDYCIKREYLI | −2.77 | 1.74E−02 |
| | | | | CEDYKLFWVAPRNSAE | | |
| 303. | BUB1B | BUB1B_131 | 780 | MFWVAPRNSAELTVIKVSSQPVPWD | −2.30 | 2.01E−02 |
| | | | | FYINLKLKERLNEDFD | | |
| 304. | C10orf90 | C10orf90105 | 624 | MFTIPHPLSDNLFKPKERCISEKEM | −1.77 | 8.72E−02 |
| | | | | HMRSKRIYDNLPEVKK | | |
| 305. | C10orf90 | C10orf90106 | 630 | MLSDNLFKPKERCISEKEMHMRSKR | −2.97 | 5.76E−03 |
| | | | | IYDNLPEVKKKKEEQR | | |
| 306. | CACNA1A | CACNA1A_27 | 156 | MFAFHKGSYLRNGWNVMDFVVVLTG | −1.87 | 1.96E−02 |
| | | | | ILATVGTEFDLRTLRA | | |
| 307. | CACNA1A | CACNA1A_28 | 162 | MSYLRNGWNVMDFVVVLTGILATVG | −2.88 | 1.23E−02 |
| | | | | TEFDLRTLRAVRVLRP | | |
| 308. | CARD11 | CARD1160 | 354 | MKHRMNTVMLQLEEVERERDQAFHS | −2.66 | 2.25E−02 |
| | | | | RDEAQTQYSQCLIEKD | | |
| 309. | CARD11 | CARD1161 | 360 | MVMLQLEEVERERDQAFHSRDEAQT | −3.34 | 6.41E−03 |
| | | | | QYSQCLIEKDKYRKQI | | |
| 310. | CARD11 | CARD1162 | 366 | MEVERERDQAFHSRDEAQTQYSQCL | −2.49 | 3.02E−02 |
| | | | | IEKDKYRKQIRELEEK | | |
| 311. | CARD11 | CARD11167 | 996 | MCKSDIVTRDEFLRRQKTETIIYSR | −4.18 | 5.43E−04 |
| | | | | EKNPNAFECIAPANIE | | |
| 312. | CARD11 | CARD11168 | 1002 | MTRDEFLRRQKTETIIYSREKNPNA | −2.41 | 2.24E−02 |
| | | | | FECIAPANIEAVAAKN | | |
| 313. | CARD11 | CARD11174 | 1038 | MAAKNKHCLLEAGIGCTRDLIKSNI | −3.68 | 2.07E−03 |
| | | | | YPIVLFIRVCEKNIKR | | |
| 314. | CARD11 | CARD11176 | 1050 | MIGCTRDLIKSNIYPIVLFIRVCEK | −3.66 | 2.15E−03 |
| | | | | NIKRFRKLLPRPETEE | | |
| 315. | CARD11 | CARD11178 | 1062 | MYPIVLFIRVCEKNIKRFRKLLPRP | −2.53 | 2.88E−02 |
| | | | | ETEEEFLRVCRLKEKE | | |
| 316. | CARS1 | CARS1_27 | 156 | MFDILRRVLKDYFKFDVFYCMNITD | −3.13 | 8.74E−03 |
| | | | | IDDKIIKRARQNHLFE | | |
| 317. | CARS1 | CARS1_28 | 162 | MVLKDYFKFDVFYCMNITDIDDKII | −3.39 | 3.53E−03 |
| | | | | KRARQNHLFEQYREKR | | |
| 318. | CARS1 | CARS1_33 | 192 | MHLFEQYREKRPEAAQLLEDVQAAL | −2.21 | 2.31E−02 |
| | | | | KPFSVKLNETTDPDKK | | |
| 319. | CARS1 | CARS1_37 | 216 | MKPFSVKLNETTDPDKKQMLERIQH | −2.11 | 5.04E−02 |
| | | | | AVQLATEPLEKAVQSR | | |
| 320. | CARS1 | CARS1_38 | 222 | MLNETTDPDKKQMLERIQHAVQLAT | −1.94 | 5.78E−02 |
| | | | | EPLEKAVQSRLTGEEV | | |
| 321. | CARS1 | CARS1_41 | 240 | MAVQLATEPLEKAVQSRLTGEEVNS | −2.24 | 1.34E−02 |
| | | | | CVEVLLEEAKDLLSDW | | |
| 322. | CARS1 | CARS1_43 | 252 | MVQSRLTGEEVNSCVEVLLEEAKDL | −3.07 | 3.04E−03 |
| | | | | LSDWLDSTLGCDVTDN | | |
| 323. | CASP8 | CASP8_8 | 42 | MMLFQRLQEKRMLEESNLSFLKELL | −1.98 | 5.29E−02 |
| | | | | FRINRLDLLITYLNTR | | |
| 324. | CASP8 | CASP8_9 | 48 | MQEKRMLEESNLSFLKELLFRINRL | −2.41 | 2.67E−02 |
| | | | | DLLITYLNTRKEEMER | | |
| 325. | CASP8 | CASP8_10 | 54 | MEESNLSFLKELLFRINRLDLLITY | −2.08 | 4.39E−02 |
| | | | | LNTRKEEMERELQTPG | | |
| 326. | CBL | CBL_6 | 30 | MDAFQPHHHHHHHLSPHPPGTVDKK | −1.84 | 5.25E−02 |
| | | | | MVEKCWKLMDKVVRLC | | |
| 327. | CBL | CBL_37 | 216 | MSGLEAMALKSTIDLTCNDYISVFE | −2.09 | 3.59E−02 |
| | | | | FDIFTRLFQPWSSLLR | | |
| 328. | CBL | CBL_40 | 234 | MYISVFEFDIFTRLFQPWSSLLRNW | −2.11 | 5.02E−02 |
| | | | | NSLAVTHPGYMAFLTY | | |
| 329. | CBWD3 | CBWD328 | 162 | MDGIITIVDSKYGLKHLTEEKPDGL | −3.11 | 4.54E−03 |
| | | | | INEATRQVALADIILI | | |
| 330. | CBWD3 | CBWD329 | 168 | MVDSKYGLKHLTEEKPDGLINEATR | −3.22 | 4.78E−03 |
| | | | | OVALADIILINKTDLV | | |
| 331. | CBWD3 | CBWD3_30 | 174 | MLKHLTEEKPDGLINEATROVALAD | −3.33 | 3.93E−03 |
| | | | | IILINKTDLVPEEDVK | | |
| 332. | CBWD3 | CBWD331 | 180 | MEKPDGLINEATRQVALADIILINK | −2.96 | 8.89E−03 |
| | | | | TDLVPEEDVKKLRTTI | | |
| 333. | CCDC6 | CCDC611 | 60 | MNRLASLQQENKVLKIELETYKLKC | −3.40 | 3.47E−03 |
| | | | | KALQEENRDLRKASVT | | |
| 334. | CCDC6 | CCDC612 | 66 | MQQENKVLKIELETYKLKCKALQEE | −1.77 | 8.47E−02 |
| | | | | NRDLRKASVTIQARAE | | |
| 335. | CCN4 | CCN4_29 | 168 | MHPRRVSIPGHCCEQWVCEDDAKRP | −2.20 | 7.47E−03 |
| | | | | RKTAPRDTGAFDAVGE | | |
| 336. | CCN4 | CCN4_30 | 174 | MIPGHCCEQWVCEDDAKRPRKTAPR | −1.86 | 4.36E−02 |
| | | | | DTGAFDAVGEVEAWHR | | |
| 337. | CCNB1 | CCNB148 | 282 | MRQMEMKILRALNFGLGRPLPLHFL | −2.00 | 6.42E−02 |
| | | | | RRASKIGEVDVEQHTL | | |
| 338. | CCNB1 | CCNB150 | 294 | MFGLGRPLPLHFLRRASKIGEVDVE | −1.86 | 5.59E−02 |
| | | | | QHTLAKYLMELTMLDY | | |
| 339. | CCND1 | CCND141 | 240 | MPDCLRACQEQIEALLESSLRQAQQ | −2.60 | 2.29E−02 |
| | | | | NMDPKAAEEEEEEEEE | | |
| 340. | CCND1 | CCND142 | 246 | MCQEQIEALLESSLRQAQQNMDPKA | −3.23 | 5.00E−04 |
| | | | | AEEEEEEEEEVDLACT | | |
| 341. | CCNL1 | CCNL1_66 | 390 | MSASRSRSRTRSRSRSHTPRRHYNN | −3.58 | 2.50E−03 |
| | | | | RRSRSGTYSSRSRSRS | | |
| 342. | CCNL1 | CCNL1_67 | 396 | MSRTRSRSRSHTPRRHYNNRRSRSG | −3.54 | 2.68E−03 |
| | | | | TYSSRSRSRSRSHSES | | |
| 343. | CCNL1 | CCNL1_71 | 420 | MTYSSRSRSRSRSHSESPRRHHNHG | −3.34 | 3.82E−03 |
| | | | | SPHLKAKHTRDDLKSS | | |
| 344. | CCNL1 | CCNL1_73 | 432 | MHSESPRRHHNHGSPHLKAKHTRDD | −2.50 | 2.93E−02 |
| | | | | LKSSNRHGHKRKKSRS | | |
| 345. | CD70 | CD70_9 | 48 | MPLESLGWDVAELQLNHTGPQQDPR | −4.01 | 1.05E−03 |
| | | | | LYWQGGPALGRSFLHG | | |
| 346. | CD70 | CD70_14 | 78 | MPALGRSFLHGPELDKGQLRIHRDG | −1.89 | 2.92E−03 |
| | | | | IYMVHIQVTLAICSST | | |
| 347. | CD79B | CD79B_3 | 12 | MWMVALLLLLSAEPVPAARSEDRYR | −3.01 | 6.77E−03 |
| | | | | NPKGSACSRIWQSPRF | | |
| 348. | CD79B | CD79B_4 | 18 | MLLLSAEPVPAARSEDRYRNPKGSA | −2.29 | 1.06E−02 |
| | | | | CSRIWQSPRFIARKRG | | |
| 349. | CDC73 | CDC73_55 | 324 | MEGASARKTQTPAAQPVPRPVSQAR | −2.01 | 3.06E−02 |
| | | | | PPPNQKKGSRTPIIII | | |
| 350. | CDC73 | CDC73_56 | 330 | MKTQTPAAQPVPRPVSQARPPPNQK | −2.29 | 2.06E−02 |
| | | | | KGSRTPIIIIPAATTS | | |
| 351. | CDCA8 | CDCA89 | 48 | MEVDNLYNIEILRLPKALREMNWLD | −2.64 | 1.82E−02 |
| | | | | YFALGGNKQALEEAAT | | |
| 352. | CDCA8 | CDCA8_12 | 66 | MEMNWLDYFALGGNKQALEEAATAD | −2.55 | 2.10E−02 |
| | | | | LDITEINKLTAEAIQT | | |
| 353. | CDH1 | CDH1_55 | 324 | MVISVVTTGLDRESFPAYTLVVQAA | −2.86 | 1.61E−02 |
| | | | | DLQGEGLSTTATAVIT | | |
| 354. | CDH1 | CDH1_58 | 342 | MLVVQAADLQGEGLSTTATAVITVT | −1.97 | 1.04E−02 |
| | | | | DTNDNPPIFNPTTYKG | | |
| 355. | CDH1 | CDH1_59 | 348 | MDLQGEGLSTTATAVITVTDTNDNP | −2.49 | 2.64E−02 |
| | | | | PIFNPTTYKGQVPENE | | |
| 356. | CDK12 | CDK12_16 | 90 | MLDRRENDERRGSDRSDRLHKHRHH | −2.08 | 4.43E−02 |
| | | | | QHRRSRDLKAKQTEK | | |
| 357. | CDK12 | CDK12_17 | 96 | MDERRGSDRSDRLHKHRHHQHRRSR | −1.89 | 5.90E−02 |
| | | | | DLLKAKQTEKEKSQEV | | |
| 358. | CDK12 | CDK12_18 | 102 | MDRSDRLHKHRHHQHRRSRDLLKAK | −2.73 | 2.04E−02 |
| | | | | QTEKEKSQEVSSKSGS | | |
| 359. | CDK12 | CDK12_19 | 108 | MHKHRHHQHRRSRDLLKAKQTEKEK | −2.11 | 5.12E−02 |
| | | | | SQEVSSKSGSMKDRIS | | |
| 360. | CDK12 | CDK12_75 | 444 | MKKLPRSVKLEKSAPDTELVNVTHL | −1.80 | 4.08E−02 |
| | | | | NTEVKNSSDTGKVKLD | | |
| 361. | CDK12 | CDK12_81 | 480 | MVKLDENSEKHLVKDLKAQGTRDSK | −2.18 | 2.41E−02 |
| | | | | PIALKEEIVTPKETET | | |
| 362. | CDK12 | CDK12_82 | 486 | MSEKHLVKDLKAQGTRDSKPIALKE | −2.06 | 2.82E−02 |
| | | | | EIVTPKETETSEKETP | | |
| 363. | CDK12 | CDK12_118 | 702 | MKICCPRYGERRQTESDWGKRCVDK | −1.85 | 8.26E−02 |
| | | | | FDIIGIIGEGTYGQVY | | |
| 364. | CDK12 | CDK12_121 | 720 | MKRCVDKFDIIGIIGEGTYGQVYKA | −2.69 | 1.39E−02 |
| | | | | KDKDTGELVALKKVRL | | |
| 365. | CDK12 | CDK12_172 | 1026 | MKMAPPDLPHWQDCHELWSKKRRRQ | −2.11 | 5.35E−02 |
| | | | | RQSGVVVEEPPPSKTS | | |
| 366. | CDK12 | CDK12_173 | 1032 | MLPHWQDCHELWSKKRRRQRQSGVV | −1.90 | 7.08E−02 |
| | | | | VEEPPPSKTSRKETTS | | |
| 367. | CDK12 | CDK12_175 | 1044 | MKKRRRQRQSGVVVEEPPPSKTSRK | −1.90 | 6.90E−02 |
| | | | | ETTSGTSTEPVKNSSP | | |
| 368. | CDK12 | CDK12_189 | 1128 | MLSIPQMAQLLNIHSNPEMQQQLEA | −5.02 | 8.89E−05 |
| | | | | LNQSISALTEATSQQQ | | |
| 369. | CDK12 | CDK12_190 | 1134 | MAQLLNIHSNPEMQQQLEALNQSIS | −5.08 | 7.45E−05 |
| | | | | ALTEATSQQQDSETMA | | |
| 370. | CDK12 | CDK12_191 | 1140 | MHSNPEMQQQLEALNQSISALTEAT | −3.17 | 2.60E−03 |
| | | | | SQQQDSETMAPEESLK | | |
| 371. | CDK12 | CDK12_219 | 1308 | MHEHQALRPMEYSTRPRPNRTYGNT | −2.28 | 2.09E−02 |
| | | | | DGPETGFSAIDTDERN | | |
| 372. | CDK12 | CDK12_223 | 1332 | MDGPETGFSAIDTDERNSGPALTES | −2.16 | 4.06E−02 |
| | | | | LVQTLVKNRTFSGSLS | | |
| 373. | CDK12 | CDK12_224 | 1338 | MFSAIDTDERNSGPALTESLVQTLV | −1.88 | 5.38E−02 |
| | | | | KNRTFSGSLSHLGESS | | |
| 374. | CEBPA | CEBPA_17 | 96 | MPTGGGGGGDFDYPGAPAGPGGAVM | −2.71 | 1.09E−02 |
| | | | | PGGAHGPPPGYGCAAA | | |
| 375. | CEBPA | CEBPA_18 | 102 | MGGDFDYPGAPAGPGGAVMPGGAHG | −2.66 | 9.58E−03 |
| | | | | PPPGYGCAAAGYLDGR | | |
| 376. | CHD3 | CHD3_131 | 780 | MEGHTKGPFLVSAPLSTIINWEREF | −3.11 | 5.76E−03 |
| | | | | QMWAPKFYVVTYTGDK | | |
| 377. | CHD3 | CHD3_136 | 810 | MFYVVTYTGDKDSRAIIRENEFSFE | −3.72 | 1.89E−03 |
| | | | | DNAIKGGKKAFKMKRE | | |
| 378. | CHD3 | CHD3_139 | 828 | MNEFSFEDNAIKGGKKAFKMKREAQ | −2.84 | 8.92E−03 |
| | | | | VKFHVLLTSYELITID | | |
| 379. | CHD3 | CHD3_214 | 1278 | MMNEYLSSFKVAQYVVREEDKIEEI | −4.19 | 7.11E−04 |
| | | | | EREIIKQEENVDPDYW | | |
| 380. | CHD3 | CHD3_215 | 1284 | MSFKVAQYVVREEDKIEEIEREIIK | −4.52 | 3.34E−04 |
| | | | | QEENVDPDYWEKLLRH | | |
| 381. | CHD3 | CHD3_233 | 1392 | MDKPLPPLLARVGGNIEVLGFNTRQ | −1.97 | 1.71E−03 |
| | | | | RKAFLNAVMRWGMPPQ | | |
| 382. | CHD4 | CHD4_64 | 378 | MIILCDTCPRAYHMVCLDPDMEKAP | −3.05 | 1.65E−03 |
| | | | | EGKWSCPHCEKEGIQW | | |
| 383. | CHD4 | CHD4_65 | 384 | MCPRAYHMVCLDPDMEKAPEGKWSC | −3.14 | 7.63E−03 |
| | | | | PHCEKEGIQWEAKEDN | | |
| 384. | CHD4 | CHD4_171 | 1020 | MLFPVAAMEAPKMPNGMYDGSALIR | −3.45 | 3.17E−03 |
| | | | | ASGKLLLLQKMLKNLK | | |
| 385. | CHD4 | CHD4_172 | 1026 | MMEAPKMPNGMYDGSALIRASGKLL | −2.02 | 6.35E−02 |
| | | | | LLQKMLKNLKEGGHRV | | |
| 386. | CHD5 | CHD5_213 | 1272 | MTDDTELQNMNEYLSSFKVAQYVVR | −2.59 | 2.00E−02 |
| | | | | EEDGVEEVEREIIKQE | | |
| 387. | CHD5 | CHD5_215 | 1284 | MLSSFKVAQYVVREEDGVEEVEREI | −2.85 | 8.19E−03 |
| | | | | IKQEENVDPDYWEKLL | | |
| 388. | CHD5 | CHD5_223 | 1332 | MEDLARNLGKGKRIRKQVNYNDASQ | −2.08 | 2.77E−02 |
| | | | | EDQEWQDELSDNQSEY | | |
| 389. | CHD5 | CHD5_270 | 1614 | MERAREERPEETEKAPPSPEQLPRE | −2.69 | 1.14E−02 |
| | | | | EVLPEKEKILDKLELS | | |
| 390. | CHD5 | CHD5_272 | 1626 | MKAPPSPEQLPREEVLPEKEKILDK | −4.56 | 3.06E−04 |
| | | | | LELSLIHSRGDSSELR | | |
| 391. | CHD8 | CHD8_45 | 264 | MRKRNTSSDNSDVEVMPAQSPREDE | −2.42 | 3.01E−02 |
| | | | | ESSIQKRRSNRQVKRK | | |
| 392. | CHD8 | CHD8_46 | 270 | MSDNSDVEVMPAQSPREDEESSIQK | −2.36 | 3.20E−02 |
| | | | | RRSNRQVKRKKYTEDL | | |
| 393. | CHD8 | CHD8_47 | 276 | MEVMPAQSPREDEESSIQKRRSNRQ | −3.67 | 2.38E−03 |
| | | | | VKRKKYTEDLDIKITD | | |
| 394. | CHD8 | CHD8_78 | 462 | MEPVIYYLVKWCSLPYEDSTWELKE | −3.36 | 3.74E−03 |
| | | | | DVDEGKIREFKRIQSR | | |
| 395. | CHD8 | CHD8_79 | 468 | MLVKWCSLPYEDSTWELKEDVDEGK | −3.82 | 1.56E−03 |
| | | | | IREFKRIQSRHPELKR | | |
| 396. | CHD8 | CHD8_80 | 474 | MLPYEDSTWELKEDVDEGKIREFKR | −3.41 | 3.41E−03 |
| | | | | IQSRHPELKRVNRPQA | | |
| 397. | CHD8 | CHD8_81 | 480 | MTWELKEDVDEGKIREFKRIQSRHP | −1.76 | 8.40E−02 |
| | | | | ELKRVNRPQASAWKKL | | |
| 398. | CHD8 | CHD8_170 | 1014 | MGIQQFSKKEIEDLLRKGAYAAIME | −2.27 | 2.10E−02 |
| | | | | EDDEGSKFCEEDIDQI | | |
| 399. | CHD8 | CHD8_174 | 1038 | MEDDEGSKFCEEDIDQILLRRTTTI | −2.63 | 1.24E−02 |
| | | | | TIESEGKGSTFAKASF | | |
| 400. | CHD8 | CHD8_175 | 1044 | MKFCEEDIDQILLRRTTTITIESEG | −2.65 | 2.21E−02 |
| | | | | KGSTFAKASFVASENR | | |
| 401. | CHD8 | CHD8_205 | 1224 | MIWDLISPAENGKTKELQNHSGLSI | −2.51 | 2.79E−02 |
| | | | | PVPRGRKGKKVKSQST | | |
| 402. | CHD8 | CHD8_207 | 1236 | MTKELQNHSGLSIPVPRGRKGKKVK | −2.36 | 3.78E−02 |
| | | | | SQSTFDIHKADWIRKY | | |
| 403. | CHD8 | CHD8_208 | 1242 | MHSGLSIPVPRGRKGKKVKSQSTFD | −2.03 | 6.22E−02 |
| | | | | IHKADWIRKYNPDTLF | | |
| 404. | CHD8 | CHD8_210 | 1254 | MKGKKVKSQSTFDIHKADWIRKYNP | −3.44 | 3.21E−03 |
| | | | | DTLFQDESYKKHLKHQ | | |
| 405. | CHD8 | CHD8_211 | 1260 | MSQSTFDIHKADWIRKYNPDTLFQD | −3.39 | 3.56E−03 |
| | | | | ESYKKHLKHQCNKVLL | | |
| 406. | CHD8 | CHD8_212 | 1266 | MIHKADWIRKYNPDTLFQDESYKKH | −1.92 | 6.27E−02 |
| | | | | LKHQCNKVLLRVRMLY | | |
| 407. | CHD8 | CHD8_213 | 1272 | MIRKYNPDTLFQDESYKKHLKHQCN | −3.70 | 1.99E−03 |
| | | | | KVLLRVRMLYYLRQEV | | |
| 408. | CHD8 | CHD8_215 | 1284 | MESYKKHLKHQCNKVLLRVRMLYYL | −2.71 | 1.09E−02 |
| | | | | RQEVIGDQAEKVLGGA | | |
| 409. | CHD8 | CHD8_216 | 1290 | MLKHQCNKVLLRVRMLYYLRQEVIG | −2.58 | 1.34E−02 |
| | | | | DQAEKVLGGAIASEID | | |
| 410. | CHEK2 | CHEK2_34 | 198 | MFVFFDLTVDDQSVYPKALRDEYIM | −2.50 | 2.88E−02 |
| | | | | SKTLGSGACGEVKLAF | | |
| 411. | CHEK2 | CHEK2_35 | 204 | MTVDDQSVYPKALRDEYIMSKTLGS | −2.99 | 6.96E−03 |
| | | | | GACGEVKLAFERKTCK | | |
| 412. | CHEK2 | CHEK2_36 | 210 | MVYPKALRDEYIMSKTLGSGACGEV | −2.69 | 1.85E−02 |
| | | | | KLAFERKTCKKVAIKI | | |
| 413. | CIC | CIC_44 | 258 | MAHFKAHPDWKWCNKDRKKSSSEAK | −2.28 | 4.31E−02 |
| | | | | PTSLGLAGGHKETRER | | |
| 414. | CIC | CIC_46 | 270 | MNKDRKKSSSEAKPTSLGLAGGHKE | −2.34 | 1.60E−02 |
| | | | | TRERSMSETGTAAAPG | | |
| 415. | CNBD1 | CNBD119 | 108 | MNNIAVHVQRAHGGHILYRPKRATE | −1.76 | 9.19E−02 |
| | | | | KFEEFLAILKKLPIHR | | |
| 416. | CNBD1 | CNBD120 | 114 | MVQRAHGGHILYRPKRATEKFEEFL | −1.88 | 7.85E−02 |
| | | | | AILKKLPIHRTPYEHK | | |
| 417. | CNBD1 | CNBD121 | 120 | MGHILYRPKRATEKFEEFLAILKKL | −2.07 | 6.01E−02 |
| | | | | PIHRTPYEHKTVWKFL | | |
| 418. | CNBD1 | CNBD164 | 378 | MLRSNKVKRSQKLVYMGKLKEKESF | −2.91 | 1.26E−02 |
| | | | | GEISVLLQVPFTCTII | | |
| 419. | CNBD1 | CNBD165 | 384 | MKRSQKLVYMGKLKEKESFGEISVL | −2.79 | 1.57E−02 |
| | | | | LQVPFTCTIITKKEVE | | |
| 420. | CNBD1 | CNBD166 | 390 | MVYMGKLKEKESFGEISVLLQVPFT | −4.03 | 1.00E−03 |
| | | | | CTIITKKEVEMALIED | | |
| 421. | COL5A1 | COL5A197 | 576 | MGSGGLKGEPGDVGPQGPRGVQGPP | −2.98 | 1.22E−02 |
| | | | | GPAGKPGRRGRAGSDG | | |
| 422. | COL5A1 | COL5A199 | 588 | MGPQGPRGVQGPPGPAGKPGRRGRA | −2.59 | 2.27E−02 |
| | | | | GSDGARGMPGQTGPKG | | |
| 423. | COL5A1 | COL5A1103 | 612 | MGSDGARGMPGQTGPKGDRGFDGLA | −1.93 | 7.28E−02 |
| | | | | GLPGEKGHRGDPGPSG | | |
| 424. | COL5A1 | COL5A1118 | 702 | MGQPGPKGNVGPQGEPGPPGQQGNP | −2.71 | 1.09E−02 |
| | | | | GAQGLPGPQGAIGPPG | | |
| 425. | COL5A1 | COL5A1124 | 738 | MGPPGEKGPLGKPGLPGMPGADGPP | −1.76 | 5.80E−02 |
| | | | | GHPGKEGPPGEKGGQG | | |
| 426. | CREB3L3 | CREB3L322 | 126 | MHPGNSCSTTTPGPVIQVPEASVTI | −1.91 | 6.08E−02 |
| | | | | DLEMWSPGGRICAEKP | | |
| 427. | CREB3L3 | CREB3L327 | 156 | MPGGRICAEKPADPVDLSPRCNLTV | −2.47 | 2.77E−03 |
| | | | | KDLLLSGSSGDLQQHH | | |
| 428. | CREBBP | CREBBP224 | 1338 | MEDRVNKFLRRQNHPEAGEVFVRVV | −2.03 | 2.96E−02 |
| | | | | ASSDKTVEVKPGMKSR | | |
| 429. | CREBBP | CREBBP225 | 1344 | MFLRRQNHPEAGEVFVRVVASSDKT | −2.80 | 9.58E−03 |
| | | | | VEVKPGMKSRFVDSGE | | |
| 430. | CREBBP | CREBBP377 | 2256 | MQGSSMGQMAAQMGQLGQMGQPGLG | −1.91 | 4.09E−03 |
| | | | | ADSTPNIQQALQQRIL | | |
| 431. | CREBBP | CREBBP379 | 2268 | MGQLGQMGQPGLGADSTPNIQQALQ | −2.51 | 1.48E−02 |
| | | | | QRILQQQQMKQQIGSP | | |
| 432. | CREBBP | CREBBP380 | 2274 | MGQPGLGADSTPNIQQALQQRILQQ | −2.41 | 1.73E−02 |
| | | | | QQMKQQIGSPGQPNPM | | |
| 433. | CRY1 | CRY1_43 | 252 | MPYLRFGCLSCRLFYFKLTDLYKKV | −1.87 | 7.08E−02 |
| | | | | KKNSSPPLSLYGQLLW | | |
| 434. | CRY1 | CRY1_44 | 258 | MCLSCRLFYFKLTDLYKKVKKNSSP | −2.36 | 3.66E−02 |
| | | | | PLSLYGQLLWREFFYT | | |
| 435. | CRY1 | CRY1_72 | 426 | MDYIRRYLPVLRGFPAKYIYDPWNA | −3.13 | 9.47E−03 |
| | | | | PEGIQKVAKCLIGVNY | | |
| 436. | CRY1 | CRY1_73 | 432 | MLPVLRGFPAKYIYDPWNAPEGIQK | −2.37 | 3.29E−02 |
| | | | | VAKCLIGVNYPKPMVN | | |
| 437. | CRY1 | CRY1_74 | 438 | MFPAKYIYDPWNAPEGIQKVAKCLI | −2.34 | 3.31E−02 |
| | | | | GVNYPKPMVNHAEASR | | |
| 438. | CRY1 | CRY1_82 | 486 | MIYQQLSRYRGLGLLASVPSNPNGN | −2.61 | 2.46E−02 |
| | | | | GGFMGYSAENIPGCSS | | |
| 439. | CRY1 | CRY1_83 | 492 | MRYRGLGLLASVPSNPNGNGGFMGY | −2.97 | 7.28E−03 |
| | | | | SAENIPGCSSSGSCSQ | | |
| 440. | CSDE1 | CSDE1_34 | 198 | MEFTIKDRNGKEVATDVRLLPQGTV | −1.92 | 7.29E−02 |
| | | | | IFEDISIEHFEGTVTK | | |
| 441. | CSDE1 | CSDE1_35 | 204 | MRNGKEVATDVRLLPQGTVIFEDIS | −2.02 | 5.95E−02 |
| | | | | IEHFEGTVTKVIPKVP | | |
| 442. | CSDE1 | CSDE1_91 | 540 | MNKVSAEKVNKTHSVNGITEEADPT | −2.40 | 3.56E−02 |
| | | | | IYSGKVIRPLRSVDPT | | |
| 443. | CSDE1 | CSDE1_93 | 552 | MSVNGITEEADPTIYSGKVIRPLRS | −4.47 | 3.72E−04 |
| | | | | VDPTQTEYQGMIEIVE | | |
| 444. | CSDE1 | CSDE1_96 | 570 | MIRPLRSVDPTQTEYQGMIEIVEEG | −2.81 | 6.54E−03 |
| | | | | DMKGEVYPFGIVGMAN | | |
| 445. | CSDE1 | CSDE1_110 | 654 | MGFINYEVGDSKKLFFHVKEVQDGI | −3.43 | 3.25E−03 |
| | | | | ELQAGDEVEFSVILNQ | | |
| 446. | CSDE1 | CSDE1_111 | 660 | MVGDSKKLFFHVKEVQDGIELQAGD | −3.47 | 3.04E−03 |
| | | | | EVEFSVILNQRTGKCS | | |
| 447. | CSDE1 | CSDE1_112 | 666 | MLFFHVKEVQDGIELQAGDEVEFSV | −2.99 | 1.22E−02 |
| | | | | ILNQRTGKCSACNVWR | | |
| 448. | CSNK2A3 | CSNK2A3_38 | 222 | MASMIFRKEPFFHGRDNYDQLVRIA | −1.95 | 6.66E−02 |
| | | | | KFLGTEDLYGYIDKYN | | |
| 449. | CSNK2A3 | CSNK2A3_39 | 228 | MKEPFFHGRDNYDQLVRIAKFLGTE | −1.95 | 6.39E−02 |
| | | | | DLYGYIDKYNIELDPR | | |
| 450. | CTNNB1 | CTNNB129 | 168 | MNKAAVMVHQLSKKEASRHAIMRSP | −1.93 | 7.02E−02 |
| | | | | QMVSAIVRTMQNTNDV | | |
| 451. | CTNNB1 | CTNNB1_30 | 174 | MVHQLSKKEASRHAIMRSPQMVSAI | −3.30 | 4.15E−03 |
| | | | | VRTMQNTNDVETARCT | | |
| 452. | CTNND1 | CTNND1_95 | 564 | MGSNTKSLDNNYSTPNERGDHNKTL | −2.13 | 2.56E−02 |
| | | | | DRSGDLGDMEPLKGTT | | |
| 453. | CTNND1 | CTNND1_96 | 570 | MLDNNYSTPNERGDHNKTLDRSGDL | −2.05 | 2.87E−02 |
| | | | | GDMEPLKGTTPLMQKI | | |
| 454. | CUL1 | CUL1_56 | 330 | MDGLGELKKLLETHIHNQGLAAIEK | −2.88 | 8.35E−03 |
| | | | | CGEAALNDPKMYVQTV | | |
| 455. | CUL1 | CUL1_57 | 336 | MKKLLETHIHNQGLAAIEKCGEAAL | −2.61 | 1.58E−02 |
| | | | | NDPKMYVQTVLDVHKK | | |
| 456. | CUL1 | CUL1_58 | 342 | MHIHNQGLAAIEKCGEAALNDPKMY | −2.11 | 2.63E−02 |
| | | | | VQTVLDVHKKYNALVM | | |
| 457. | CUL3 | CUL3_67 | 396 | MKKGVKGLTEQEVETILDKAMVLFR | −2.80 | 7.63E−03 |
| | | | | FMQEKDVFERYYKQHL | | |
| 458. | CUL3 | CUL3_68 | 402 | MLTEQEVETILDKAMVLFRFMQEKD | −2.69 | 1.87E−02 |
| | | | | VFERYYKQHLARRLLT | | |
| 459. | CUL3 | CUL3_69 | 408 | METILDKAMVLFRFMQEKDVFERYY | −3.24 | 4.61E−03 |
| | | | | KQHLARRLLTNKSVSD | | |
| 460. | CUL3 | CUL3_70 | 414 | MAMVLFRFMQEKDVFERYYKQHLAR | −3.20 | 4.96E−03 |
| | | | | RLLTNKSVSDDSEKNM | | |
| 461. | CUL4A | CUL4A_23 | 132 | MLKFLEETNCLYAAEGQRLMQEREV | −2.29 | 3.12E−02 |
| | | | | PEYLNHVSKRLEEEGD | | |
| 462. | CUL4A | CUL4A_24 | 138 | MTNCLYAAEGQRLMQEREVPEYLNH | −2.52 | 1.47E−02 |
| | | | | VSKRLEEEGDRVITYL | | |
| 463. | CUL4A | CUL4A_25 | 144 | MAEGQRLMQEREVPEYLNHVSKRLE | −2.19 | 2.37E−02 |
| | | | | EEGDRVITYLDHSTQK | | |
| 464. | CUL4A | CUL4A_27 | 156 | MPEYLNHVSKRLEEEGDRVITYLDH | −2.73 | 1.98E−02 |
| | | | | STQKPLIACVEKQLLG | | |
| 465. | CUL4A | CUL4A_35 | 204 | MKGLDHLLDENRVPDLAQMYQLFSR | −1.88 | 3.63E−02 |
| | | | | VRGGQQALLQHWSEYI | | |
| 466. | CUL4A | CUL4A_36 | 210 | MLDENRVPDLAQMYQLFSRVRGGQQ | −1.80 | 4.08E−02 |
| | | | | ALLQHWSEYIKTFGTA | | |
| 467. | CUL4A | CUL4A_56 | 330 | MRTLDKIMILFRFIHGKDVFEAFYK | −2.62 | 1.55E−02 |
| | | | | KDLAKRLLVGKSASVD | | |
| 468. | CUL4A | CUL4A_57 | 336 | MMILFRFIHGKDVFEAFYKKDLAKR | −2.32 | 2.97E−02 |
| | | | | LLVGKSASVDAEKSML | | |
| 469. | CUL4A | CUL4A_58 | 342 | MIHGKDVFEAFYKKDLAKRLLVGKS | −3.02 | 6.68E−03 |
| | | | | ASVDAEKSMLSKLKHE | | |
| 470. | CUL4A | CUL4A_60 | 354 | MKDLAKRLLVGKSASVDAEKSMLSK | −2.13 | 2.56E−02 |
| | | | | LKHECGAAFTSKLEGM | | |
| 471. | CYLD | CYLD_1 | 0 | MMSSGLWSQEKVTSPYWEERIFYLL | −2.30 | 3.82E−02 |
| | | | | LQECSVTDKQTQKLLK | | |
| 472. | CYLD | CYLD_2 | 6 | MSQEKVTSPYWEERIFYLLLQECSV | −2.20 | 4.83E−02 |
| | | | | TDKQTQKLLKVPKGSI | | |
| 473. | CYLD | CYLD_43 | 252 | MDVLPGKESLGYFVGVDMDNPIGNW | −2.03 | 4.53E−02 |
| | | | | DGRFDGVQLCSFACVE | | |
| 474. | CYLD | CYLD_44 | 258 | MESLGYFVGVDMDNPIGNWDGRFDG | −2.48 | 2.49E−02 |
| | | | | VQLCSFACVESTILLH | | |
| 475. | CYLD | CYLD_45 | 264 | MVGVDMDNPIGNWDGRFDGVQLCSF | −3.41 | 5.58E−03 |
| | | | | ACVESTILLHINDIIP | | |
| 476. | CYLD | CYLD_59 | 348 | MRNRSELFYTLNGSSVDSQPQSKSK | −2.16 | 4.88E−02 |
| | | | | NTWYIDEVAEDPAKSL | | |
| 477. | CYLD | CYLD_60 | 354 | MFYTLNGSSVDSQPQSKSKNTWYID | −1.87 | 6.94E−02 |
| | | | | EVAEDPAKSLTEISTD | | |
| 478. | DAB2IP | DAB2IP6 | 30 | MVVIKPVHSSILGQDYCFEVTTSSG | −4.94 | 1.11E−04 |
| | | | | SKCFSCRSAAERDKWM | | |
| 479. | DAB2IP | DAB2IP8 | 42 | MQDYCFEVITSSGSKCFSCRSAAER | −1.89 | 4.54E−02 |
| | | | | DKWMENLRRAVHPNKD | | |
| 480. | DAB2IP | DAB2IP_24 | 138 | MHNLPPLRTVTVHLYRETDKKKKKE | −2.35 | 2.52E−03 |
| | | | | RNSYLGLVSLPAASVA | | |
| 481. | DAB2IP | DAB2IP25 | 144 | MRTVTVHLYRETDKKKKKERNSYLG | −3.47 | 2.71E−03 |
| | | | | LVSLPAASVAGRQFVE | | |
| 482. | DAB2IP | DAB2IP47 | 276 | MVDRCGDNEHLIFRENTLATKAIEE | −4.71 | 2.07E−04 |
| | | | | YLKLVGQKYLQDALGE | | |
| 483. | DAB2IP | DAB2IP49 | 288 | MRENTLATKAIEEYLKLVGQKYLQD | −2.54 | 9.21E−03 |
| | | | | ALGEFIKALYESDENC | | |
| 484. | DAB2IP | DAB2IP50 | 294 | MTKAIEEYLKLVGQKYLQDALGEFI | −2.88 | 8.35E03 |
| | | | | KALYESDENCEVDPSK | | |
| 485. | DAB2IP | DAB2IP_154 | 918 | MAVLQDKLRISTKKLEEYETLFKCQ | −4.24 | 6.45E−04 |
| | | | | EETTQKLVLEYQARLE | | |
| 486. | DAB2IP | DAB2IP155 | 924 | MLRISTKKLEEYETLFKCQEETTQK | −2.09 | 2.70E−02 |
| | | | | LVLEYQARLEEGEERL | | |
| 487. | DAZAP1 | DAZAP16 | 30 | MSQYGEVVDCVIMKDKTTNQSRGFG | −3.38 | 5.80E−03 |
| | | | | FVKFKDPNCVGTVLAS | | |
| 488. | DAZAP1 | DAZAP17 | 36 | MVDCVIMKDKTTNQSRGFGFVKFKD | −2.14 | 3.54E−02 |
| | | | | PNCVGTVLASRPHTLD | | |
| 489. | DCC | DCC_194 | 1158 | MEMEMKNIEKPSGTDPAGRDSPIQS | −2.52 | 2.74E−02 |
| | | | | CQDLTPVSHSQSETQL | | |
| 490. | DCC | DCC_198 | 1182 | MCQDLTPVSHSQSETQLGSKSTSHS | −1.89 | 5.11E−02 |
| | | | | GQDTEEAGSSMSTLER | | |
| 491. | DDIT3 | DDIT3_11 | 60 | MAWLTEEEPEPAEVTSTSQSPHSPD | −3.43 | 3.30E−03 |
| | | | | SSQSSLAQEEEEEDQG | | |
| 492. | DDIT3 | DDIT3_12 | 66 | MEPEPAEVTSTSQSPHSPDSSQSSL | −3.05 | 1.11E−02 |
| | | | | AQEEEEEDQGRTRKRK | | |
| 493. | DDIT3 | DDIT3_20 | 114 | MAGKQRMKEKEQENERKVAQLAEEN | −3.17 | 5.20E−03 |
| | | | | ERLKQEIERLTREVEA | | |
| 494. | DDX3X | DDX3X_21 | 120 | MRGGNSRWCDKSDEDDWSKPLPPSE | −3.02 | 1.07E−02 |
| | | | | RLEQELFSGGNTGINF | | |
| 495. | DDX3X | DDX3X_96 | 570 | MLENMAYEHHYKGSSRGRSKSSRFS | −1.88 | 2.55E−02 |
| | | | | GGFGARDYROSSGASS | | |
| 496. | DDX3X | DDX3X_97 | 576 | MEHHYKGSSRGRSKSSRFSGGFGAR | −2.53 | 9.92E−03 |
| | | | | DYRQSSGASSSSFSSS | | |
| 497. | DEK | DEK_22 | 126 | MVGQFSGFPFEKGSVQYKKKEEMLK | −2.85 | 8.77E−03 |
| | | | | KFRNAMLKSICEVLDL | | |
| 498. | DEK | DEK_23 | 132 | MFPFEKGSVQYKKKEEMLKKFRNAM | −3.06 | 6.24E−03 |
| | | | | LKSICEVLDLERSGVN | | |
| 499. | DEK | DEK_24 | 138 | MSVQYKKKEEMLKKFRNAMLKSICE | −3.02 | 6.69E−03 |
| | | | | VLDLERSGVNSELVKR | | |
| 500. | DEK | DEK_25 | 144 | MKEEMLKKFRNAMLKSICEVLDLER | −2.74 | 1.55E−02 |
| | | | | SGVNSELVKRILNFLM | | |
| 501. | DHX9 | DHX9_157 | 936 | MIRFCEHKRLNMATLRMTWEAKVQL | −2.89 | 8.32E−03 |
| | | | | KEILINSGFPEDCLLT | | |
| 502. | DHX9 | DHX9_158 | 942 | MKRLNMATLRMTWEAKVQLKEILIN | −2.21 | 4.62E−02 |
| | | | | SGFPEDCLLTQVFTNT | | |
| 503. | DHX9 | DHX9_159 | 948 | MTLRMTWEAKVQLKEILINSGFPED | −2.94 | 7.55E−03 |
| | | | | CLLTQVFTNTGPDNNL | | |
| 504. | DHX9 | DHX9_163 | 972 | MCLLTQVFTNTGPDNNLDVVISLLA | −2.88 | 6.74E−03 |
| | | | | FGVYPNVCYHKEKRKI | | |
| 505. | DIAPH2 | DIAPH237 | 216 | MLDELEKLLDKKQQENIDKKNQYKL | −2.17 | 2.43E−02 |
| | | | | IQCLKAFMNNKFGLQR | | |
| 506. | DIAPH2 | DIAPH238 | 222 | MLLDKKQQENIDKKNQYKLIQCLKA | −2.26 | 2.15E−02 |
| | | | | FMNNKFGLQRILGDER | | |
| 507. | DIAPH2 | DIAPH239 | 228 | MQENIDKKNQYKLIQCLKAFMNNKF | −1.98 | 2.63E−02 |
| | | | | GLQRILGDERSLLLLA | | |
| 508. | DIAPH2 | DIAPH240 | 234 | MKNQYKLIQCLKAFMNNKFGLQRIL | −2.21 | 2.31E−02 |
| | | | | GDERSLLLLARAIDPK | | |
| 509. | DIAPH2 | DIAPH244 | 258 | MGDERSLLLLARAIDPKQPNMMTEI | −1.81 | 8.04E−02 |
| | | | | VKILSAICIVGEENIL | | |
| 510. | DIAPH2 | DIAPH246 | 270 | MIDPKQPNMMTEIVKILSAICIVGE | −2.12 | 5.12E−02 |
| | | | | ENILDKLLGAITTAAE | | |
| 511. | DIAPH2 | DIAPH247 | 276 | MNMMTEIVKILSAICIVGEENILDK | −2.02 | 6.17E−02 |
| | | | | LLGAITTAAERNNRER | | |
| 512. | DIAPH2 | DIAPH261 | 360 | MLRSGLKTMLPDLKEKENDELDIQL | −3.00 | 8.32E−03 |
| | | | | KVFDENKEDDLTELSH | | |
| 513. | DIAPH2 | DIAPH262 | 366 | MTMLPDLKEKENDELDIQLKVFDEN | −2.31 | 4.08E−02 |
| | | | | KEDDLTELSHRLNDIR | | |
| 514. | DIAPH2 | DIAPH263 | 372 | MKEKENDELDIQLKVFDENKEDDLT | −2.39 | 3.55E−02 |
| | | | | ELSHRLNDIRAEMDDM | | |
| 515. | DIAPH2 | DIAPH2_69 | 408 | MMDDMNEVYHLLYNMLKDTAAENYF | −2.78 | 1.71E−02 |
| | | | | LSILQHFLLIRNDYYI | | |
| 516. | DIAPH2 | DIAPH270 | 414 | MVYHLLYNMLKDTAAENYFLSILQH | −2.94 | 1.21E−02 |
| | | | | FLLIRNDYYIRPQYYK | | |
| 517. | DIAPH2 | DIAPH271 | 420 | MNMLKDTAAENYFLSILQHFLLIRN | −2.65 | 2.31E−02 |
| | | | | DYYIRPQYYKIIEECV | | |
| 518. | DIAPH2 | DIAPH272 | 426 | MAAENYFLSILQHFLLIRNDYYIRP | −3.35 | 3.76E−03 |
| | | | | QYYKIIEECVSQIVLH | | |
| 519. | DICER1 | DICER123 | 132 | MWTKERWNQEFTKHQVLIMTCYVAL | −1.77 | 7.73E− |
| | | | | NVLKNGYLSLSDINLL | | |
| | | | | | | 02 |
| 520. | DICER1 | DICER124 | 138 | MNQEFTKHQVLIMTCYVALNVLKNG | −2.05 | 5.67E−02 |
| | | | | YLSLSDINLLVFDECH | | |
| 521. | DICER1 | DICER127 | 156 | MNVLKNGYLSLSDINLLVFDECHLA | −2.06 | 3.24E−02 |
| | | | | ILDHPYREIMKLCENC | | |
| 522. | DICER1 | DICER170 | 414 | MSEDDDEDEEIEEKEKPETNFPSPF | −1.88 | 5.56E−02 |
| | | | | TNILCGIIFVERRYTA | | |
| 523. | DICER1 | DICER171 | 420 | MDEEIEEKEKPETNFPSPFTNILCG | −2.45 | 1.61E−02 |
| | | | | IIFVERRYTAVVLNRL | | |
| 524. | DICER1 | DICER172 | 426 | MKEKPETNFPSPFTNILCGIIFVER | −2.20 | 2.34E−02 |
| | | | | RYTAVVLNRLIKEAGK | | |
| 525. | DICER1 | DICER1_80 | 474 | MSSNFITGHGIGKNQPRNKQMEAEF | −2.99 | 7.05E−03 |
| | | | | RKQEEVLRKFRAHETN | | |
| 526. | DICER1 | DICER187 | 516 | MIATSIVEEGVDIPKCNLVVRFDLP | −2.01 | 4.66E−02 |
| | | | | TEYRSYVQSKGRARAP | | |
| 527. | DICER1 | DICER188 | 522 | MEEGVDIPKCNLVVRFDLPTEYRSY | −1.95 | 7.14E−02 |
| | | | | VQSKGRARAPISNYIM | | |
| 528. | DICER1 | DICER189 | 528 | MPKCNLVVRFDLPTEYRSYVQSKGR | −2.06 | 5.98E−02 |
| | | | | ARAPISNYIMLADTDK | | |
| 529. | DICER1 | DICER1204 | 1218 | MIQNLYSYENQPQPSDECTLLSNKY | −3.45 | 3.17E−03 |
| | | | | LDGNANKSTSDGSPVM | | |
| 530. | DICER1 | DICER1205 | 1224 | MYENQPQPSDECTLLSNKYLDGNAN | −2.69 | 1.95E−02 |
| | | | | KSTSDGSPVMAVMPGT | | |
| 531. | DICER1 | DICER1219 | 1308 | MGFNLERLEMLGDSFLKHAITTYLF | −1.93 | 2.36E−02 |
| | | | | CTYPDAHEGRLSYMRS | | |
| 532. | DICER1 | DICER1220 | 1314 | MLEMLGDSFLKHAITTYLFCTYPDA | −2.04 | 2.05E−02 |
| | | | | HEGRLSYMRSKKVSNC | | |
| 533. | DICER1 | DICER1222 | 1326 | MITTYLFCTYPDAHEGRLSYMRSKK | −2.75 | 1.03E−02 |
| | | | | VSNCNLYRLGKKKGLP | | |
| 534. | DLC1 | DLC1_72 | 426 | MHQNKSPPKVKAEDGMQCLQLKETL | −2.01 | 3.05E−02 |
| | | | | ATQEPTDNQVRLRKRK | | |
| 535. | DLC1 | DLC1_73 | 432 | MPKVKAEDGMQCLQLKETLATQEPT | −1.85 | 3.80E−02 |
| | | | | DNQVRLRKRKEIREDR | | |
| 536. | DLC1 | DLC1_99 | 588 | MGFPQYAQLYEDFLFPIDISLVKRE | −2.76 | 1.85E−02 |
| | | | | HDFLDRDAIEALCRRL | | |
| 537. | DLC1 | DLC1_100 | 594 | MQLYEDFLFPIDISLVKREHDFLDR | −3.24 | 3.65E−03 |
| | | | | DAIEALCRRLNTLNKC | | |
| 538. | DLC1 | DLC1_262 | 1566 | MCALLLTSVDHDRAPVVGVRVNVLL | −1.89 | 6.13E−02 |
| | | | | SRYLIEPCGPGKSKLT | | |
| 539. | DLC1 | DLC1_263 | 1572 | MSVDHDRAPVVGVRVNVLLSRYLIE | −3.32 | 5.09E−03 |
| | | | | PCGPGKSKLTYMCRVD | | |
| 540. | DLC1 | DLC1_301 | 1800 | MRFAQRSVNVCLLLAEWQXMSKVDS | −4.37 | 4.75E−04 |
| | | | | VSGTLRHLQEGDXCIR | | |
| 541. | DLC1 | DLC1_302 | 1806 | MVNVCLLLAEWQXMSKVDSVSGTLR | −2.13 | 1.29E−02 |
| | | | | HLQEGDXCIRFSLTWN | | |
| 542. | DLC1 | DLC1_309 | 1848 | MLPLNPPRKGAQHPCPFMGIFFRSY | −2.08 | 3.50E−02 |
| | | | | XAYSFSTNLFCTLIVI | | |
| 543. | DLC1 | DLC1_310 | 1854 | MRKGAQHPCPFMGIFFRSYXAYSFS | −2.39 | 2.17E−02 |
| | | | | TNLFCTLIVIGWLLHP | | |
| 544. | DLC1 | DLC1_311 | 1860 | MPCPFMGIFFRSYXAYSFSTNLFCT | −2.69 | 1.31E−02 |
| | | | | LIVIGWLLHPFQMQFI | | |
| 545. | DLC1 | DLC1_312 | 1866 | MIFFRSYXAYSFSTNLFCTLIVIGW | −2.99 | 8.79E−03 |
| | | | | LLHPFQMQFIFRPDCK | | |
| 546. | DLC1 | DLC1_325 | 1944 | MRDIKTEHDNTYSXIKHQHFFPVRI | −2.28 | 1.71E−02 |
| | | | | LCIFVFVTETPXDTMF | | |
| 547. | DLC1 | DLC1_326 | 1950 | MHDNTYSXIKHQHFFPVRILCIFVF | −2.47 | 1.57E−02 |
| | | | | VTETPXDTMFGNIGNY | | |
| 548. | DLC1 | DLC1_327 | 1956 | MXIKHQHFFPVRILCIFVFVTETPX | −2.67 | 1.16E−02 |
| | | | | DTMFGNIGNYVCPKEI | | |
| 549. | DLC1 | DLC1_328 | 1962 | MFFPVRILCIFVFVTETPXDTMFGN | −3.14 | 9.34E−03 |
| | | | | IGNYVCPKEIPVNLTH | | |
| 550. | DLC1 | DLC1_329 | 1968 | MLCIFVFVTETPXDTMFGNIGNYVC | −3.52 | 2.17E−03 |
| | | | | PKEIPVNLTHLQKAFS | | |
| 551. | DLC1 | DLC1_334 | 1998 | MNLTHLQKAFSPPLVVNGIPESHMC | −2.47 | 3.13E−02 |
| | | | | LFIGLVLISKXVFWFY | | |
| 552. | DLC1 | DLC1_335 | 2004 | MKAFSPPLVVNGIPESHMCLFIGLV | −2.56 | 2.31E−02 |
| | | | | LISKXVFWFYDFLSYF | | |
| 553. | DLC1 | DLC1_336 | 2010 | MLVVNGIPESHMCLFIGLVLISKXV | −3.03 | 5.25E−03 |
| | | | | FWFYDFLSYFIFPTFL | | |
| 554. | DLC1 | DLC1_346 | 2070 | MNHSKTQQEENQPKPVKFFRTFRRV | −2.60 | 1.29E−02 |
| | | | | VFIQVLLXWXGLTQET | | |
| 555. | DLC1 | DLC1_347 | 2076 | MQEENQPKPVKFFRTFRRWFIQVLL | −1.88 | 7.55E−02 |
| | | | | XWXGLTQETVLVNLL | | |
| 556. | DLC1 | DLC1_348 | 2082 | MKPVKFFRTFRRVVFIQVLLXWXGL | −3.31 | 4.09E−03 |
| | | | | TQETVLVNLLCMDQK | | |
| 557. | DLC1 | DLC1_349 | 2088 | MRTFRRVVFIQVLLXWXGLTQETVL | −3.36 | 3.71E−03 |
| | | | | VNLLCMDQKXIMYEXE | | |
| 558. | DLC1 | DLC1_350 | 2094 | MVFIQVLLXWXGLTQETVLVNLLCM | −3.15 | 5.36E−03 |
| | | | | DQKXIMYEXELXEGFL | | |
| 559. | DLC1 | DLC1_351 | 2100 | MLXWXGLTQETVLVNLLCMDQKXIM | −3.29 | 4.17E−03 |
| | | | | YEXELXEGFLFCYNLV | | |
| 560. | DLC1 | DLC1_353 | 2112 | MVNLLCMDQKXIMYEXELXEGFLFC | −2.54 | 1.41E−02 |
| | | | | YNLVTIFSVISKVLXR | | |
| 561. | DLC1 | DLC1_354 | 2118 | MDQKXIMYEXELXEGFLFCYNLVTI | −2.00 | 5.24E−02 |
| | | | | FSVISKVLXRILGQGI | | |
| 562. | DLC1 | DLC1_355 | 2124 | MYEXELXEGFLFCYNLVTIFSVISK | −2.07 | 3.49E−02 |
| | | | | VLXRILGQGIRLEFXN | | |
| 563. | DLC1 | DLC1_356 | 2130 | MEGFLFCYNLVTIFSVISKVLXRIL | −2.48 | 1.55E−02 |
| | | | | GQGIRLEFXNLSILDI | | |
| 564. | DLC1 | DLC1_372 | 2226 | MRYSFGFCTSRLFSSYCKYTLREYS | −1.94 | 3.38E−02 |
| | | | | LSEENKXKEEVYNLLY | | |
| 565. | DLC1 | DLC1_373 | 2232 | MCTSRLFSSYCKYTLREYSLSEENK | −2.32 | 1.97E−02 |
| | | | | XKEEVYNLLYICTDYN | | |
| 566. | DLC1 | DLC1_374 | 2238 | MSSYCKYTLREYSLSEENKXKEEVY | −2.30 | 2.01E−02 |
| | | | | NLLYICTDYNQXVHYY | | |
| 567. | DLC1 | DLC1_375 | 2244 | MTLREYSLSEENKXKEEVYNLLYIC | −2.24 | 2.20E−02 |
| | | | | TDYNQXVHYYXMFKVR | | |
| 568. | DMBT1 | DMBT1_11 | 60 | MAEGSPISLESTLESTVAEGSLIPS | −2.01 | 4.33E−02 |
| | | | | ESTLESTVAEGSDSGL | | |
| 569. | DMBT1 | DMBT1_12 | 66 | MSLESTLESTVAEGSLIPSESTLES | −3.30 | 6.94E−03 |
| | | | | TVAEGSDSGLALRLVN | | |
| 570. | DMBT1 | DMBT1_13 | 72 | MESTVAEGSLIPSESTLESTVAEGS | −1.86 | 4.07E−02 |
| | | | | DSGLALRLVNGDGRCQ | | |
| 571. | DMBT1 | DMBT1_234 | 1398 | MDLVISTWNGYYECRPQITPNLVIF | −2.94 | 1.27E−02 |
| | | | | TIPYSGCGTFKQADND | | |
| 572. | DMBT1 | DMBT1_235 | 1404 | MWNGYYECRPQITPNLVIFTIPYSG | −3.41 | 5.64E−03 |
| | | | | CGTFKQADNDTIDYSN | | |
| 573. | DMTF1 | DMTF1_52 | 306 | MAAVAERVGTRSEKQCRSKWLNYLN | −3.09 | 9.12E−03 |
| | | | | WKQSGGTEWTKEDEIN | | |
| 574. | DMTF1 | DMTF1_54 | 318 | MKQCRSKWLNYLNWKQSGGTEWTKE | −2.56 | 1.37E−02 |
| | | | | DEINLILRIAELDVAD | | |
| 575. | DNMT3A | DNMT3A21 | 120 | MAAETLPEASRAVENGCCTPKEGRG | −3.30 | 4.12E−03 |
| | | | | APAEAGKEQKETNIES | | |
| 576. | DNMT3A | DNMT3A22 | 126 | MEASRAVENGCCTPKEGRGAPAEAG | −3.45 | 3.16E−03 |
| | | | | KEQKETNIESMKMEGS | | |
| 577. | DNMT3A | DNMT3A60 | 354 | MHQATYNKQPMYRKAIYEVLQVASS | −1.83 | 3.27E−02 |
| | | | | RAGKLFPVCHDSDESD | | |
| 578. | DNMT3A | DNMT3A62 | 366 | MKAIYEVLQVASSRAGKLFPVCHDS | −1.94 | 3.38E−02 |
| | | | | DESDTAKAVEVQNKPM | | |
| 579. | DNMT3A | DNMT3A_63 | 372 | MLQVASSRAGKLFPVCHDSDESDTA | −1.96 | 3.27E−02 |
| | | | | KAVEVQNKPMIEWALG | | |
| 580. | DNMT3A | DNMT3A75 | 444 | MAAAYAPPPPAKKPRKSTAEKPKVK | −2.35 | 1.88E−02 |
| | | | | EIIDERTRERLVYEVR | | |
| 581. | DNMT3A | DNMT3A_77 | 456 | MPRKSTAEKPKVKEIIDERTRERLV | −3.50 | 2.91E−03 |
| | | | | YEVRQKCRNIEDICIS | | |
| 582. | DOCK4 | DOCK436 | 210 | MCSNLGEELEVIFSLFDSKENRPIS | −1.81 | 4.01E−02 |
| | | | | ERFFLRLNRNGLPKAP | | |
| 583. | DOCK4 | DOCK437 | 216 | MELEVIFSLFDSKENRPISERFFLR | −1.93 | 3.41E−02 |
| | | | | LNRNGLPKAPDKPERH | | |
| 584. | DOCK4 | DOCK462 | 366 | MDIEQIRREYSSVFSHGVSITRKLG | −3.00 | 6.92E−03 |
| | | | | FSNIIMPGEMRNDLYI | | |
| 585. | DOCK4 | DOCK463 | 372 | MREYSSVFSHGVSITRKLGFSNIIM | −3.01 | 6.76E−03 |
| | | | | PGEMRNDLYITIERGE | | |
| 586. | DOCK4 | DOCK464 | 378 | MFSHGVSITRKLGFSNIIMPGEMRN | −2.66 | 9.64E−03 |
| | | | | DLYITIERGEFEKGGK | | |
| 587. | DOCK4 | DOCK465 | 384 | MITRKLGFSNIIMPGEMRNDLYITI | −2.58 | 1.34E−02 |
| | | | | ERGEFEKGGKSVARNV | | |
| 588. | DOCK4 | DOCK4103 | 612 | MVKFLQDTLDTLFGILDENSQKYGS | −2.71 | 1.10E−02 |
| | | | | KVFDSLVHIINLLQDS | | |
| 589. | DOCK4 | DOCK4104 | 618 | MTLDTLFGILDENSQKYGSKVFDSL | −2.71 | 8.82E−03 |
| | | | | VHIINLLQDSKFHHFK | | |
| 590. | DOCK4 | DOCK4105 | 624 | MGILDENSQKYGSKVFDSLVHIINL | −2.32 | 2.95E−02 |
| | | | | LQDSKFHHFKPVMDTY | | |
| 591. | DOCK4 | DOCK4214 | 1278 | MDRPLREFLTYPMQTEWQRKEHLHL | −1.80 | 4.52E−02 |
| | | | | TIIQNFDRGKCWENGI | | |
| 592. | DOCK4 | DOCK4216 | 1290 | MQTEWQRKEHLHLTIIQNFDRGKCW | −1.90 | 7.46E−02 |
| | | | | ENGIILCRKIAEQYES | | |
| 593. | DOCK4 | DOCK4241 | 1440 | MQREGVPDNIKSFYKVNHIWKFRYD | −2.23 | 4.61E−02 |
| | | | | RPFHKGTKDKENEFKS | | |
| 594. | DOCK4 | DOCK4242 | 1446 | MDNIKSFYKVNHIWKFRYDRPFHKG | −3.26 | 4.45E−03 |
| | | | | TKDKENEFKSLWVERT | | |
| 595. | DOCK4 | DOCK4246 | 1470 | MTKDKENEFKSLWVERTSLYLVQSL | −3.65 | 2.17E−03 |
| | | | | PGISRWFEVEKREVVE | | |
| 596. | DOCK4 | DOCK4247 | 1476 | MEFKSLWVERTSLYLVQSLPGISRW | −3.21 | 7.56E−03 |
| | | | | FEVEKREVVEMSPLEN | | |
| 597. | DYNC1LI1 | DYNC1LI1_37 | 216 | MEDKDDSVVLPLGADTLTHNLGIPV | −2.09 | 2.70E−02 |
| | | | | LVVCTKCDAISVLEKE | | |
| 598. | DYNC1LI1 | DYNC1LI1_39 | 228 | MADTLTHNLGIPVLVVCTKCDAISV | −2.17 | 4.93E−02 |
| | | | | LEKEHDYRDEHFDFIQ | | |
| 599. | DYNC1LI1 | DYNC1LI1_48 | 282 | MIYTSVKENKNIDLVYKYIVQKLYG | −2.33 | 1.94E−02 |
| | | | | FPYKIPAVVVEKDAVF | | |
| 600. | DYNC1LI1 | DYNC1LI1_49 | 288 | MENKNIDLVYKYIVQKLYGFPYKIP | −2.52 | 1.20E−02 |
| | | | | AVVVEKDAVFIPAGWD | | |
| 601. | DYNC1LI1 | DYNC1LI1_50 | 294 | MLVYKYIVQKLYGFPYKIPAVVVEK | −2.05 | 2.89E−02 |
| | | | | DAVFIPAGWDNDKKIG | | |
| 602. | DYNC1LI1 | DYNC1LI1_51 | 300 | MVQKLYGFPYKIPAVVVEKDAVFIP | −2.00 | 6.64E−02 |
| | | | | AGWDNDKKIGILHENF | | |
| 603. | DYNC1LI1 | DYNC1LI1_52 | 306 | MFPYKIPAVVVEKDAVFIPAGWDND | −2.22 | 4.56E−02 |
| | | | | KKIGILHENFQTLKAE | | |
| 604. | DYNC1LI1 | DYNC1LI1_53 | 312 | MAVVVEKDAVFIPAGWDNDKKIGIL | −2.05 | 6.18E−02 |
| | | | | HENFQTLKAEDNFEDI | | |
| 605. | DYNC1LI1 | DYNC1LI1_71 | 420 | MSPIPAGSKKIDPNMKAGATSEGVL | −3.42 | 3.31E−03 |
| | | | | ANFFNSLLSKKTGSPG | | |
| 606. | DYNC1LI1 | DYNC1LI1_72 | 426 | MSKKIDPNMKAGATSEGVLANFFNS | −3.32 | 3.98E−03 |
| | | | | LLSKKTGSPGGPGVSG | | |
| 607. | EEF1A1 | EEF1A124 | 138 | MLAYTLGVKQLIVGVNKMDSTEPPY | −2.04 | 2.90E−02 |
| | | | | SQKRYEEIVKEVSTYI | | |
| 608. | EEF1A1 | EEF1A125 | 144 | MVKQLIVGVNKMDSTEPPYSQKRYE | −2.02 | 2.48E−02 |
| | | | | EIVKEVSTYIKKIGYN | | |
| 609. | EIF1AX | EIF1AX_9 | 48 | MAMCFDGVKRLCHIRGKLRKKVWIN | −2.11 | 2.63E−02 |
| | | | | TSDIILVGLRDYQDNK | | |
| 610. | EIF1AX | EIF1AX_10 | 54 | MVKRLCHIRGKLRKKVWINTSDIIL | −1.96 | 3.27E−02 |
| | | | | VGLRDYQDNKADVILK | | |
| 611. | EIF1AX | EIF1AX_15 | 84 | MQDNKADVILKYNADEARSLKAYGE | −4.40 | 4.48E−04 |
| | | | | LPEHAKINETDTFGPG | | |
| 612. | EIF1AX | EIF1AX_16 | 90 | MVILKYNADEARSLKAYGELPEHAK | −4.32 | 3.91E−04 |
| | | | | INETDTFGPGDDDEIQ | | |
| 613. | ELF4 | ELF4_37 | 216 | MALLQDRNTCPKYIKWTOREKGIFK | −2.68 | 1.15E−02 |
| | | | | LVDSKAVSKLWGKQKN | | |
| 614. | ELF4 | ELF4_39 | 228 | MIKWTOREKGIFKLVDSKAVSKLWG | −2.44 | 1.64E−02 |
| | | | | KQKNKPDMNYETMGRA | | |
| 615. | ELL | ELL_17 | 96 | MQQYVSSHGEVHLDCLGSIQDKITV | −2.44 | 2.49E−03 |
| | | | | CATDDSYQKARQSMAQ | | |
| 616. | ELL | ELL_20 | 114 | MQDKITVCATDDSYQKARQSMAQAE | −3.04 | 6.86E−04 |
| | | | | EETRSRSAIVIKAGGR | | |
| 617. | ENTPD5 | ENTPD554 | 318 | MEEVQRGSFYAFSYYYDRAVDTDMI | −2.33 | 1.95E−02 |
| | | | | DYEKGGILKVEDFERK | | |
| 618. | ENTPD5 | ENTPD555 | 324 | MSFYAFSYYYDRAVDTDMIDYEKGG | −2.23 | 1.85E−02 |
| | | | | ILKVEDFERKAREVCD | | |
| 619. | ENTPD5 | ENTPD556 | 330 | MYYYDRAVDTDMIDYEKGGILKVED | −1.99 | 2.60E−02 |
| | | | | FERKAREVCDNLENFT | | |
| 620. | EP300 | EP300_21 | 120 | MMVKSPMTQAGLTSPNMGMGTSGPN | −2.76 | 1.58E−02 |
| | | | | QGPTQSTGMMNSPVNQ | | |
| 621. | EP300 | EP300_27 | 156 | MPVNQPAMGMNTGMNAGMNPGMLAA | −3.50 | 2.87E−03 |
| | | | | GNGQGIMPNQVMNGSI | | |
| 622. | EP300 | EP300_76 | 450 | MPNLSTVSQIDPSSIERAYAALGLP | −2.57 | 2.52E−02 |
| | | | | YQVNQMPTQPQVQAKN | | |
| 623. | EP300 | EP300_78 | 462 | MSIERAYAALGLPYQVNQMPTQPQV | −2.10 | 5.73E−02 |
| | | | | QAKNQQNQQPGQSPQG | | |
| 624. | EP300 | EP300_79 | 468 | MAALGLPYQVNQMPTQPQVQAKNQQ | −2.82 | 9.27E−03 |
| | | | | NQQPGQSPQGMRPMSN | | |
| 625. | EP300 | EP300_93 | 552 | MLGPMPTAAQPSTTGIRKQWHEDIT | −2.02 | 4.31E−02 |
| | | | | QDLRNHLVHKLVQAIF | | |
| 626. | EP300 | EP300_97 | 576 | MQDLRNHLVHKLVQAIFPTPDPAAL | −1.78 | 8.02E−02 |
| | | | | KDRRMENLVAYARKVE | | |
| 627. | EP300 | EP300_99 | 588 | MQAIFPTPDPAALKDRRMENLVAYA | −3.20 | 4.89E−03 |
| | | | | RKVEGDMYESANNRAE | | |
| 628. | EP300 | EP300_108 | 642 | MELEEKRRTRLQKQNMLPNAAGMVP | −2.35 | 1.88E−02 |
| | | | | VSMNPGPNMGQPQPGM | | |
| 629. | EP300 | EP300_109 | 648 | MRTRLQKQNMLPNAAGMVPVSMNPG | −2.10 | 5.30E−02 |
| | | | | PNMGQPQPGMTSNGPL | | |
| 630. | EP300 | EP300_112 | 666 | MVSMNPGPNMGQPQPGMTSNGPLPD | −1.82 | 6.60E−02 |
| | | | | PSMIRGSVPNQMMPRI | | |
| 631. | EP300 | EP300_197 | 1176 | MCYGKQLCTIPRDATYYSYQNRYHF | −3.96 | 1.18E−03 |
| | | | | CEKCFNEIQGESVSLG | | |
| 632. | EP300 | EP300_199 | 1188 | MATYYSYQNRYHFCEKCFNEIQGES | −2.85 | 8.85E−03 |
| | | | | VSLGDDPSQPQTTINK | | |
| 633. | EP300 | EP300_200 | 1194 | MQNRYHFCEKCFNEIQGESVSLGDD | −2.32 | 3.73E−02 |
| | | | | PSQPQTTINKEQFSKR | | |
| 634. | EP300 | EP300_201 | 1200 | MCEKCFNEIQGESVSLGDDPSQPQT | −1.86 | 8.14E−02 |
| | | | | TINKEQFSKRKNDTLD | | |
| 635. | EP300 | EP300_337 | 2016 | MSVAQHGQPLNMAPQPGLGQVGISP | −1.87 | 2.45E−02 |
| | | | | LKPGTVSQQALQNLLR | | |
| 636. | EP300 | EP300_339 | 2028 | MPQPGLGQVGISPLKPGTVSQQALQ | −4.43 | 4.10E−04 |
| | | | | NLLRTLRSPSSPLQQQ | | |
| 637. | EPAS1 | EPAS1_45 | 264 | MIGYHPEELLGRSAYEFYHALDSEN | −2.49 | 1.96E−02 |
| | | | | MTKSHQNLCTKGQVVS | | |
| 638. | EPAS1 | EPAS1_47 | 276 | MAYEFYHALDSENMTKSHQNLCTKG | −2.67 | 1.04E−02 |
| | | | | QVVSGQYRMLAKHGGY | | |
| 639. | EPB41L3 | EPB41L347 | 276 | MMTPAEAEMHFLENAKKLSMYGVDL | −2.39 | 3.52E−02 |
| | | | | HHAKDSEGVEIMLGVC | | |
| 640. | EPB41L3 | EPB41L348 | 282 | MEMHFLENAKKLSMYGVDLHHAKDS | −3.55 | 2.61E−03 |
| | | | | EGVEIMLGVCASGLLI | | |
| 641. | EPB41L3 | EPB41L359 | 348 | MIKIRPGEFEQFESTIGFKLPNHRA | −3.08 | 6.04E−03 |
| | | | | AKRLWKVCVEHHTFFR | | |
| 642. | EPB41L3 | EPB41L361 | 360 | MSTIGFKLPNHRAAKRLWKVCVEHH | −3.14 | 5.41E−03 |
| | | | | TFFRLLLPEAPPKKFL | | |
| 643. | EPHA2 | EPHA2_151 | 900 | MSEGVPFRTVSEWLESIKMQQYTEH | −4.22 | 6.70E−04 |
| | | | | FMAAGYTAIEKVVQMT | | |
| 644. | EPHA2 | EPHA2_156 | 930 | MTAIEKVVQMTNDDIKRIGVRLPGH | −1.76 | 2.27E−02 |
| | | | | QKRIAYSLLGLKDQVN | | |
| 645. | EPHA3 | EPHA3_24 | 138 | MQFTKIDTIAADESFTQMDLGDRIL | −3.20 | 3.90E−03 |
| | | | | KLNTEIREVGPVNKKG | | |
| 646. | EPHA3 | EPHA3_27 | 156 | MLGDRILKLNTEIREVGPVNKKGFY | −2.76 | 1.75E−02 |
| | | | | LAFQDVGACVALVSVR | | |
| 647. | EPHA3 | EPHA3_28 | 162 | MKLNTEIREVGPVNKKGFYLAFQDV | −2.13 | 3.97E−02 |
| | | | | GACVALVSVRVYFKKC | | |
| 648. | EPHA3 | EPHA3_29 | 168 | MREVGPVNKKGFYLAFQDVGACVAL | −3.67 | 2.09E−03 |
| | | | | VSVRVYFKKCPFTVKN | | |
| 649. | EPHA3 | EPHA3_102 | 606 | MQTVHEFAKELDATNISIDKVVGAG | −2.10 | 3.80E−02 |
| | | | | EFGEVCSGRLKLPSKK | | |
| 650. | EPHA3 | EPHA3_103 | 612 | MAKELDATNISIDKVVGAGEFGEVC | −2.65 | 1.88E−02 |
| | | | | SGRLKLPSKKEISVAI | | |
| 651. | EPHA3 | EPHA3_104 | 618 | MTNISIDKVVGAGEFGEVCSGRLKL | −2.53 | 2.13E−02 |
| | | | | PSKKEISVAIKTLKVG | | |
| 652. | EPHA3 | EPHA3_153 | 912 | MFRTTGDWLNGVWTAHCKEIFTGVE | −2.68 | 1.15E−02 |
| | | | | YSSCDTIAKISTDDMK | | |
| 653. | EPHA3 | EPHA3_154 | 918 | MWLNGVWTAHCKEIFTGVEYSSCDT | −2.33 | 3.93E−02 |
| | | | | IAKISTDDMKKVGVTV | | |
| 654. | EPHA3 | EPHA3_155 | 924 | MTAHCKEIFTGVEYSSCDTIAKIST | −2.48 | 3.06E−02 |
| | | | | DDMKKVGVTVVGPQKK | | |
| 655. | EPS15 | EPS15_3 | 12 | MSSGNPVYEKYYRQVDTGNTGRVLA | −2.17 | 3.13E−02 |
| | | | | SDAAAFLKKSGLPDLI | | |
| 656. | EPS15 | EPS15_6 | 30 | MTGRVLASDAAAFLKKSGLPDLILG | −2.12 | 5.52E−02 |
| | | | | KIWDLADTDGKGILNK | | |
| 657. | EPS15 | EPS15_7 | 36 | MSDAAAFLKKSGLPDLILGKIWDLA | −2.54 | 2.54E−02 |
| | | | | DTDGKGILNKQEFFVA | | |
| 658. | ERBB2 | ERBB2_55 | 324 | MEDGTQRCEKCSKPCARVCYGLGME | −2.30 | 2.01E−02 |
| | | | | HLREVRAVTSANIQEF | | |
| 659. | ERBB2 | ERBB2_56 | 330 | MCEKCSKPCARVCYGLGMEHLREVR | −2.27 | 2.10E−02 |
| | | | | AVTSANIQEFAGCKKI | | |
| 660. | ERBB4 | ERBB4_31 | 180 | MNGSSGCGRCHKSCTGRCWGPTENH | −3.35 | 5.60E−03 |
| | | | | CQTLTRTVCAEQCDGR | | |
| 661. | ERBB4 | ERBB4_35 | 204 | MCQTLTRTVCAEQCDGRCYGPYVSD | −1.83 | 2.66E−02 |
| | | | | CCHRECAGGCSGPKDT | | |
| 662. | ERBB4 | ERBB4_84 | 498 | MEGMVCNHLCSSDGCWGPGPDQCLS | −1.77 | 8.33E−02 |
| | | | | CRRFSRGRICIESCNL | | |
| 663. | ERBB4 | ERBB4_86 | 510 | MGCWGPGPDQCLSCRRFSRGRICIE | −3.43 | 3.25E−03 |
| | | | | SCNLYDGEFREFENGS | | |
| 664. | ERBB4 | ERBB4_87 | 516 | MPDQCLSCRRFSRGRICIESCNLYD | −3.19 | 8.65E−03 |
| | | | | GEFREFENGSICVECD | | |
| 665. | ERRFI1 | ERRFI1_1 | 0 | MMSIAGVAAQEIRVPLKTGFLHNGR | −3.91 | 1.31E−03 |
| | | | | AMGNMRKTYWSSRSEF | | |
| 666. | ERRFI1 | ERRFI1_2 | 6 | MAAQEIRVPLKTGFLHNGRAMGNMR | −3.92 | 1.26E−03 |
| | | | | KTYWSSRSEFKNNFLN | | |
| 667. | ERRFI1 | ERRFI1_60 | 354 | MDPKYVSSKALQRQNSEGSASKVPC | −4.10 | 8.66E−04 |
| | | | | ILPIIENGKKVSSTHY | | |
| 668. | ERRFI1 | ERRFI1_63 | 372 | MASKVPCILPIIENGKKVSSTHYYL | −4.12 | 9.07E−04 |
| | | | | LPERPPYLDKYEKFFR | | |
| 669. | ERRFI1 | ERRFI1_64 | 378 | MILPIIENGKKVSSTHYYLLPERPP | −3.83 | 2.23E−03 |
| | | | | YLDKYEKFFREAEETN | | |
| 670. | ERRFI1 | ERRFI1_65 | 384 | MNGKKVSSTHYYLLPERPPYLDKYE | −3.90 | 1.32E−03 |
| | | | | KFFREAEETNGGAQIQ | | |
| 671. | ERRFI1 | ERRFI1_66 | 390 | MSTHYYLLPERPPYLDKYEKFFREA | −2.74 | 1.04E−02 |
| | | | | EETNGGAQIQPLPADC | | |
| 672. | ERRFI1 | ERRFI1_67 | 396 | MLPERPPYLDKYEKFFREAEETNGG | −2.19 | 4.65E−02 |
| | | | | AQIQPLPADCGISSAT | | |
| 673. | ETAA1 | ETAA1_60 | 354 | MSCVTSCTKEPETSNKYIDAFTTSD | −1.92 | 6.79E−02 |
| | | | | FEDDWENLLGSEPFAM | | |
| 674. | ETAA1 | ETAA1_61 | 360 | MTKEPETSNKYIDAFTTSDFEDDWE | −3.52 | 2.77E−03 |
| | | | | NLLGSEPFAMQNIDMP | | |
| 675. | ETAA1 | ETAA1_95 | 564 | MPNQTSASKVGSFFDDWNDPSFANE | −2.33 | 2.91E−02 |
| | | | | IIKACHQLDNTWEADD | | |
| 676. | ETAA1 | ETAA1_96 | 570 | MSKVGSFFDDWNDPSFANEIIKACH | −3.24 | 7.55E−03 |
| | | | | QLDNTWEADDVDDDLL | | |
| 677. | ETAA1 | ETAA1_97 | 576 | MFDDWNDPSFANEIIKACHQLDNTW | −2.53 | 2.67E−02 |
| | | | | EADDVDDDLLYQACDD | | |
| 678. | ETAA1 | ETAA1_98 | 582 | MPSFANEIIKACHQLDNTWEADDVD | −3.21 | 8.20E−03 |
| | | | | DDLLYQACDDIERLTQ | | |
| 679. | ETAA1 | ETAA1_136 | 810 | MNLNTTVGFSKFTFTRMKNSQILSQ | −2.00 | 2.55E−02 |
| | | | | FNQNCITGSMSDTKIT | | |
| 680. | ETAA1 | ETAA1_137 | 816 | MGFSKFTFTRMKNSQILSQFNQNCI | −2.20 | 2.34E−02 |
| | | | | TGSMSDTKITQGVEKK | | |
| 681. | ETAA1 | ETAA1_138 | 822 | MFTRMKNSQILSQFNQNCITGSMSD | −1.80 | 4.06E−02 |
| | | | | TKITQGVEKKKGVNPL | | |
| 682. | ETAA1 | ETAA1_139 | 828 | MSQILSQFNQNCITGSMSDTKITQG | −2.17 | 2.43E−02 |
| | | | | VEKKKGVNPLLEEAVG | | |
| 683. | ETS2 | ETS2_13 | 72 | MPCSKAVMSQALKATFSGFKKEQRR | −1.94 | 3.38E−02 |
| | | | | LGIPKNPWLWSEQQVC | | |
| 684. | ETS2 | ETS2_17 | 96 | MLGIPKNPWLWSEQQVCQWLLWATN | −2.55 | 1.40E−02 |
| | | | | EFSLVNVNLQRFGMNG | | |
| 685. | ETS2 | ETS2_22 | 126 | MVNLQRFGMNGQMLCNLGKERFLEL | −1.79 | 6.68E−02 |
| | | | | APDFVGDILWEHLEQM | | |
| 686. | ETV6 | ETV6_13 | 72 | MAENEFSLRPIDSNTFEMNGKALLL | −2.34 | 1.92E−02 |
| | | | | LTKEDFRYRSPHSGDV | | |
| 687. | ETV6 | ETV6_15 | 84 | MNTFEMNGKALLLLTKEDFRYRSPH | −2.50 | 1.50E−02 |
| | | | | SGDVLYELLQHILKQR | | |
| 688. | ETV6 | ETV6_60 | 354 | MENFIRWEDKESKIFRIVDPNGLAR | −2.67 | 1.16E−02 |
| | | | | LWGNHKNRTNMTYEKM | | |
| 689. | ETV6 | ETV6_61 | 360 | MEDKESKIFRIVDPNGLARLWGNHK | −2.70 | 1.11E−02 |
| | | | | NRTNMTYEKMSRALRH | | |
| 690. | EVI2A | EVI2A_2 | 6 | MYLHLAFLMTTVFSLSPGTKANYTR | −1.95 | 5.98E−02 |
| | | | | LWANSTSSWDSVIQNK | | |
| 691. | EVI2A | EVI2A_3 | 12 | MLMTTVFSLSPGTKANYTRLWANST | −1.89 | 6.70E−02 |
| | | | | SSWDSVIQNKTGRNQN | | |
| 692. | EVI2A | EVI2A_7 | 36 | MSSWDSVIQNKTGRNQNENINTNPI | −3.13 | 5.53E−03 |
| | | | | TPEVDYKGNSTNMPET | | |
| 693. | EVI2A | EVI2A_8 | 42 | MIQNKTGRNQNENINTNPITPEVDY | −2.80 | 1.40E−02 |
| | | | | KGNSTNMPETSHIVAL | | |
| 694. | EVI2A | EVI2A_9 | 48 | MRNQNENINTNPITPEVDYKGNSTN | −2.74 | 1.54E−02 |
| | | | | MPETSHIVALTSKSEQ | | |
| 695. | EVI2A | EVI2A_31 | 180 | MASGLWPAESDTWKRTKQLTGPNLV | −1.76 | 8.05E−02 |
| | | | | MQSTGVLTATRERKDE | | |
| 696. | EVI2A | EVI2A_32 | 186 | MAESDTWKRTKQLTGPNLVMQSTGV | −2.58 | 2.64E−02 |
| | | | | LTATRERKDEEGTEKL | | |
| 697. | EVI2A | EVI2A_33 | 192 | MKRTKQLTGPNLVMQSTGVLTATRE | −2.60 | 2.54E−02 |
| | | | | RKDEEGTEKLTNKQIG | | |
| 698. | EVI2B | EVI2B_33 | 192 | MQTPQKNNYNSIAAILIGVLLTSML | −2.33 | 3.65E−02 |
| | | | | VAIIIIVLWKCLRKPV | | |
| 699. | EVI2B | EVI2B_34 | 198 | MNYNSIAAILIGVLLTSMLVAIIII | −2.74 | 1.05E−02 |
| | | | | VLWKCLRKPVLNDQNW | | |
| 700. | EWSR1 | EWSR1_60 | 354 | MDEDSDNSAIYVQGLNDSVTLDDLA | −2.51 | 1.48E−02 |
| | | | | DFFKQCGVVKMNKRTG | | |
| 701. | EWSR1 | EWSR1_63 | 372 | MTLDDLADFFKQCGVVKMNKRTGQP | −2.16 | 5.01E−02 |
| | | | | MIHIYLDKETGKPKGD | | |
| 702. | EWSR1 | EWSR1_65 | 384 | MGVVKMNKRTGQPMIHIYLDKETGK | −2.35 | 3.79E−02 |
| | | | | PKGDATVSYEDPPTAK | | |
| 703. | EWSR1 | EWSR1_66 | 390 | MKRTGQPMIHIYLDKETGKPKGDAT | −2.42 | 3.39E−02 |
| | | | | VSYEDPPTAKAAVEWF | | |
| 704. | EWSR1 | EWSR1_68 | 402 | MDKETGKPKGDATVSYEDPPTAKAA | −4.00 | 1.07E−03 |
| | | | | VEWFDGKDFQGSKLKV | | |
| 705. | EXT1 | EXT1_96 | 570 | MSTTEVDFAFTVWQSFPERIVGYPA | −2.87 | 1.58E−02 |
| | | | | RSHFWDNSKERWGYTS | | |
| 706. | EXT1 | EXT1_97 | 576 | MFAFTVWQSFPERIVGYPARSHFWD | −2.92 | 1.43E−02 |
| | | | | NSKERWGYTSKWTNDY | | |
| 707. | EXT2 | EXT2_91 | 540 | MIIMLTSDELQFGYEVWREFPDRLV | −2.17 | 2.43E−02 |
| | | | | GYPGRLHLWDHEMNKW | | |
| 708. | EXT2 | EXT2_92 | 546 | MDELQFGYEVWREFPDRLVGYPGRL | −2.24 | 2.20E−02 |
| | | | | HLWDHEMNKWKYESEW | | |
| 709. | EZH2 | EZH2_75 | 444 | MFRVLIGTYYDNFCAIARLIGTKTC | −2.51 | 2.24E−02 |
| | | | | RQVYEFRVKESSIIAP | | |
| 710. | EZH2 | EZH2_78 | 462 | MIGTKTCRQVYEFRVKESSIIAPAP | −2.54 | 2.27E−02 |
| | | | | AEDVDTPPRKKKRKHR | | |
| 711. | EZH2 | EZH2_114 | 678 | MVVDATRKGNKIRFANHSVNPNCYA | −2.33 | 3.22E−02 |
| | | | | KVMMVNGDHRIGIFAK | | |
| 712. | EZH2 | EZH2_115 | 684 | MKGNKIRFANHSVNPNCYAKVMMVN | −3.32 | 3.98E−03 |
| | | | | GDHRIGIFAKRAIQTG | | |
| 713. | FAM 120A | FAM120A106 | 630 | MNRLPPEFSPVIIKEWAAYKGKSPQ | −2.08 | 5.56E−02 |
| | | | | TPELVEALAFREWTCP | | |
| 714. | FAM 120A | FAM120A109 | 648 | MKGKSPQTPELVEALAFREWTCPNL | −2.57 | 1.36E−02 |
| | | | | KRLWLGKAVEDKNRRM | | |
| 715. | FAM83B | FAM83B53 | 312 | MLASVSSQRNLFGRQDKIHKLDSSY | −3.14 | 5.46E−03 |
| | | | | FKNRGIYTLNEHDKYN | | |
| 716. | FAM83B | FAM83B54 | 318 | MQRNLFGRQDKIHKLDSSYFKNRGI | −3.15 | 5.39E−03 |
| | | | | YTLNEHDKYNIRSHGY | | |
| 717. | FAM83B | FAM83B55 | 324 | MRQDKIHKLDSSYFKNRGIYTLNEH | −2.37 | 3.71E−02 |
| | | | | DKYNIRSHGYKPHFVP | | |
| 718. | FAM83B | FAM83B56 | 330 | MKLDSSYFKNRGIYTLNEHDKYNIR | −3.08 | 4.81E−03 |
| | | | | SHGYKPHFVPNFNGPN | | |
| 719. | FAM83D | FAM83D38 | 222 | MTITGNIYYARSGTKIIGKVHEKFT | −2.13 | 3.92E−02 |
| | | | | LIDGIRVATGSYSFTW | | |
| 720. | FAM83D | FAM83D39 | 228 | MYYARSGTKIIGKVHEKFTLIDGIR | −2.13 | 3.97E−02 |
| | | | | VATGSYSFTWTDGKLN | | |
| 721. | FAM83D | FAM83D40 | 234 | MTKIIGKVHEKFTLIDGIRVATGSY | −3.61 | 3.49E−03 |
| | | | | SFTWTDGKLNSSNLVI | | |
| 722. | FAM83D | FAM83D42 | 246 | MLIDGIRVATGSYSFTWTDGKLNSS | −3.06 | 1.12E−02 |
| | | | | NLVILSGQVVEHFDLE | | |
| 723. | FAP | FAP_89 | 528 | MDRSKKYPLLIQVYGGPCSQSVRSV | −2.54 | 1.42E−02 |
| | | | | FAVNWISYLASKEGMV | | |
| 724. | FAP | FAP_90 | 534 | MPLLIQVYGGPCSQSVRSVFAVNWI | −1.81 | 6.16E−02 |
| | | | | SYLASKEGMVIALVDG | | |
| 725. | FAP | FAP_110 | 654 | MYYASVYTERFMGLPTKDDNLEHYK | −2.18 | 4.54E−02 |
| | | | | NSTVMARAEYFRNVDY | | |
| 726. | FAP | FAP_111 | 660 | MTERFMGLPTKDDNLEHYKNSTVMA | −2.49 | 2.29E−02 |
| | | | | RAEYFRNVDYLLIHGT | | |
| 727. | FAT1 | FAT1_50 | 294 | MVAGDLLQQFRTVRSFPGSKEYKVK | −3.08 | 6.08E−03 |
| | | | | AIGGIDWDSHPFGYNL | | |
| 728. | FAT1 | FAT1_51 | 300 | MQQFRTVRSFPGSKEYKVKAIGGID | −2.95 | 7.50E−03 |
| | | | | WDSHPFGYNLTLQAKD | | |
| 729. | FAT1 | FAT1_52 | 306 | MRSFPGSKEYKVKAIGGIDWDSHPF | −2.22 | 4.54E−02 |
| | | | | GYNLTLQAKDKGTPPQ | | |
| 730. | FAT1 | FAT1_65 | 384 | MPVVMVKAIPAYSHLRYVFKSTPGK | −3.38 | 2.83E−03 |
| | | | | AKFSLNYNTGLISILE | | |
| 731. | FAT1 | FAT1_66 | 390 | MAIPAYSHLRYVFKSTPGKAKFSLN | −2.49 | 3.05E−02 |
| | | | | YNTGLISILEPVKRQQ | | |
| 732. | FBXO4 | FBX04_15 | 84 | MTNHYWNETVRDPILWRYFLLRDLP | −2.01 | 6.24E−02 |
| | | | | SWSSVDWKSLPDLEIL | | |
| 733. | FBXO4 | FBXO4_16 | 90 | METVRDPILWRYFLLRDLPSWSSVD | −2.95 | 1.10E−02 |
| | | | | WKSLPDLEILKKPISE | | |
| 734. | FBXO4 | FBXO4_17 | 96 | MILWRYFLLRDLPSWSSVDWKSLPD | −2.89 | 1.43E−02 |
| | | | | LEILKKPISEVTDGAF | | |
| 735. | FBXW7 | FBXW7_97 | 576 | MLTGHQSLTSGMELKDNILVSGNAD | −2.09 | 5.61E−02 |
| | | | | STVKIWDIKTGQCLQT | | |
| 736. | FBXW7 | FBXW7_99 | 588 | MLKDNILVSGNADSTVKIWDIKTGQ | −2.23 | 4.53E−02 |
| | | | | CLQTLQGPNKHQSAVT | | |
| 737. | FER | FER_38 | 222 | MKGIFDEYSQITSLVTEEIVNVHKE | −2.21 | 2.31E−02 |
| | | | | IQMSVEQIDPSTEYNN | | |
| 738. | FER | FER_40 | 234 | MLVTEEIVNVHKEIQMSVEQIDPST | −1.82 | 8.55E−02 |
| | | | | EYNNFIDVHRTTAAKE | | |
| 739. | FER | FER_41 | 240 | MVNVHKEIQMSVEQIDPSTEYNNFI | −2.28 | 3.14E−02 |
| | | | | DVHRTTAAKEQEIEFD | | |
| 740. | FER | FER_63 | 372 | MTEAKFSAQKELLEQKVQENDGKEP | −1.94 | 6.45E−02 |
| | | | | PPVVNYEEDARSVTSM | | |
| 741. | FER | FER_64 | 378 | MAQKELLEQKVQENDGKEPPPVVNY | −2.50 | 1.50E−02 |
| | | | | EEDARSVTSMERKERL | | |
| 742. | FER | FER_105 | 624 | MGVCTQRQPVYIIMELVSGGDFLTF | −2.66 | 1.50E−02 |
| | | | | LRRKKDELKLKQLVKF | | |
| 743. | FER | FER_108 | 642 | MGDFLTFLRRKKDELKLKQLVKFSL | −2.67 | 1.02E−02 |
| | | | | DAAAGMLYLESKNCIH | | |
| 744. | FER | FER_109 | 648 | MLRRKKDELKLKQLVKFSLDAAAGM | −2.09 | 4.38E−02 |
| | | | | LYLESKNCIHRDLAAR | | |
| 745. | FGF5 | FGF5_18 | 102 | MIYPDGKVNGSHEANMLSVLEIFAV | −2.38 | 3.65E−02 |
| | | | | SQGIVGIRGVFSNKFL | | |
| 746. | FGF5 | FGF5_19 | 108 | MVNGSHEANMLSVLEIFAVSQGIVG | −2.92 | 1.34E−02 |
| | | | | IRGVFSNKFLAMSKKG | | |
| 747. | FGF5 | FGF5_20 | 114 | MANMLSVLEIFAVSQGIVGIRGVFS | −3.50 | 2.91E−03 |
| | | | | NKFLAMSKKGKLHASA | | |
| 748. | FGFR2 | FGFR2_59 | 348 | MYELPEDPKWEFPRDKLTLGKPLGE | −2.14 | 2.53E−02 |
| | | | | GCFGQVVMAEAVGIDK | | |
| 749. | FGFR2 | FGFR2_60 | 354 | MPKWEFPRDKLTLGKPLGEGCFGQV | −2.04 | 2.90E−02 |
| | | | | VMAEAVGIDKDKPKEA | | |
| 750. | FGFR2 | FGFR2_62 | 366 | MGKPLGEGCFGQVVMAEAVGIDKDK | −2.11 | 2.64E−02 |
| | | | | PKEAVTVAVKMLKDDA | | |
| 751. | FGFR3 | FGFR3_3 | 12 | MAVAIVAGASSESLGTEQRVVGRAA | −1.84 | 8.36E−02 |
| | | | | EVPGPEPGQQEQLVFG | | |
| 752. | FGFR3 | FGFR3_21 | 120 | MFSVRVTDAPSSGDDEDGEDEAEDT | −3.38 | 4.16E−03 |
| | | | | GVDTGAPYWTRPERMD | | |
| 753. | FGFR3 | FGFR3_22 | 126 | MDAPSSGDDEDGEDEAEDTGVDTGA | −3.32 | 4.28E−03 |
| | | | | PYWTRPERMDKKLLAV | | |
| 754. | FH | FH_71 | 420 | MRLLGDASVSFTENCVVGIQANTER | −1.78 | 5.34E−02 |
| | | | | INKLMNESLMLVTALN | | |
| 755. | FH | FH_73 | 432 | MNCVVGIQANTERINKLMNESLMLV | −1.90 | 2.91E−02 |
| | | | | TALNPHIGYDKAAKIA | | |
| 756. | FH | FH_74 | 438 | MQANTERINKLMNESLMLVTALNPH | −4.09 | 6.62E−04 |
| | | | | IGYDKAAKIAKTAHKN | | |
| 757. | FLCN | FLCN_43 | 252 | MCLHTSFAWLLKACGSRLTEKLLEG | −1.76 | 1.80E−02 |
| | | | | APTEDTLVQMEKLAGE | | |
| 758. | FLCN | FLCN_47 | 276 | MAPTEDTLVQMEKLAGEAGVLLPGP | −2.21 | 3.66E−02 |
| | | | | WPGWPWGGTSCLLSWQ | | |
| 759. | FLCN | FLCN_49 | 288 | MLAGEAGVLLPGPWPGWPWGGTSCL | −2.95 | 7.51E−03 |
| | | | | LSWQESLREGNAALNQ | | |
| 760. | FLNA | FLNA_91 | 540 | MPASFAVSLNGAKGAIDAKVHSPSG | −3.64 | 2.22E−03 |
| | | | | ALEECYVTEIDQDKYA | | |
| 761. | FLNA | FLNA_93 | 552 | MGAIDAKVHSPSGALEECYVTEIDQ | −2.80 | 1.55E−02 |
| | | | | DKYAVRFIPRENGVYL | | |
| 762. | FLT3 | FLT3_128 | 762 | MEDEIEYENQKRLEEEEDLNVLTFE | −2.35 | 2.85E−02 |
| | | | | DLLCFAYQVAKGMEFL | | |
| 763. | FLT3 | FLT3_129 | 768 | MENQKRLEEEEDLNVLTFEDLLCFA | −1.93 | 7.40E−02 |
| | | | | YQVAKGMEFLEFKSCV | | |
| 764. | FOX01 | FOXO1_65 | 384 | MTSLTVSTQSSPGTMMQQTPCYSFA | −2.67 | 2.26E−02 |
| | | | | PPNTSLNSPSPNYQKY | | |
| 765. | FOX01 | FOXO1_66 | 390 | MTQSSPGTMMQQTPCYSFAPPNTSL | −2.49 | 3.02E−02 |
| | | | | NSPSPNYQKYTYGQSS | | |
| 766. | FRK | FRK_50 | 294 | MVCTLEDPIYIITELMRHGSLQEYL | −1.85 | 7.99E−02 |
| | | | | QNDTGSKIHLTQQVDM | | |
| 767. | FRK | FRK_51 | 300 | MPIYIITELMRHGSLQEYLQNDTGS | −2.03 | 5.98E−02 |
| | | | | KIHLTQQVDMAAQVAS | | |
| 768. | FRK | FRK_59 | 348 | MNYIHRDLAARNVLVGEHNIYKVAD | −2.46 | 3.09E−02 |
| | | | | FGLARVFKVDNEDIYE | | |
| 769. | FRK | FRK_60 | 354 | MLAARNVLVGEHNIYKVADFGLARV | −3.00 | 6.86E−03 |
| | | | | FKVDNEDIYESRHEIK | | |
| 770. | FRK | FRK_61 | 360 | MLVGEHNIYKVADFGLARVFKVDNE | −1.78 | 9.22E−02 |
| | | | | DIYESRHEIKLPVKWT | | |
| 771. | FRK | FRK_62 | 366 | MIYKVADFGLARVFKVDNEDIYESR | −2.78 | 1.70E−02 |
| | | | | HEIKLPVKWTAPEAIR | | |
| 772. | FRK | FRK_63 | 372 | MFGLARVFKVDNEDIYESRHEIKLP | −2.57 | 2.64E−02 |
| | | | | VKWTAPEAIRSNKFSI | | |
| 773. | FRK | FRK_64 | 378 | MFKVDNEDIYESRHEIKLPVKWTAP | −2.33 | 2.92E−02 |
| | | | | EAIRSNKFSIKSDVWS | | |
| 774. | FUBP1 | FUBP1_36 | 210 | MQERAGVKMVMIQDGPQNTGADKPL | −3.24 | 4.58E−03 |
| | | | | RITGDPYKVQQAKEMV | | |
| 775. | FUBP1 | FUBP1_38 | 222 | MDGPQNTGADKPLRITGDPYKVQQA | −3.58 | 2.50E−03 |
| | | | | KEMVLELIRDQGGFRE | | |
| 776. | FUBP1 | FUBP1_39 | 228 | MGADKPLRITGDPYKVQQAKEMVLE | −3.66 | 2.15E−03 |
| | | | | LIRDQGGFREVRNEYG | | |
| 777. | FUS | FUS_69 | 408 | MGFPSGGGGGGGQQRAGDWKCPNPT | −3.71 | 1.94E−03 |
| | | | | CENMNFSWRNECNQCK | | |
| 778. | FUS | FUS_70 | 414 | MGGGGGQQRAGDWKCPNPTCENMNF | −2.82 | 9.19E−03 |
| | | | | SWRNECNQCKAPKPDG | | |
| 779. | FYN | FYN_14 | 78 | MRGGTGVTLFVALYDYEARTEDDLS | −2.22 | 2.26E−02 |
| | | | | FHKGEKFQILNSSEGD | | |
| 780. | FZD3 | FZD3_34 | 198 | MLSFARYFIGLISIICLSATLFTFL | −2.22 | 4.60E−02 |
| | | | | TFLIDVTRFRYPERPI | | |
| 781. | FZD3 | FZD3_35 | 204 | MFIGLISIICLSATLFTFLTFLIDV | −2.81 | 9.33E−03 |
| | | | | TRFRYPERPIIFYAVC | | |
| 782. | FZD3 | FZD3_36 | 210 | MIICLSATLFTFLTFLIDVTRFRYP | −2.55 | 1.72E−02 |
| | | | | ERPIIFYAVCYMMVSL | | |
| 783. | FZD3 | FZD3_37 | 216 | MTLFTFLTFLIDVTRFRYPERPIIF | −2.88 | 8.43E−03 |
| | | | | YAVCYMMVSLIFFIGF | | |
| 784. | FZD3 | FZD3_40 | 234 | MERPIIFYAVCYMMVSLIFFIGFLL | −2.38 | 3.05E−02 |
| | | | | EDRVACNASIPAQYKA | | |
| 785. | FZD3 | FZD3_55 | 324 | MEKKALLFHASAWGIPGTLTIILLA | −3.21 | 4.81E−03 |
| | | | | MNKIEGDNISGVCFVG | | |
| 786. | FZD3 | FZD3_56 | 330 | MFHASAWGIPGTLTIILLAMNKIEG | −2.24 | 2.76E−02 |
| | | | | DNISGVCFVGLYDVDA | | |
| 787. | FZD3 | FZD3_57 | 336 | MGIPGTLTIILLAMNKIEGDNISGV | −3.41 | 2.65E−03 |
| | | | | CFVGLYDVDALRYFVL | | |
| 788. | GABRA6 | GABRA6_11 | 60 | MDIYVTSFGPVSDVEMEYTMDVFFR | −3.11 | 5.79E−03 |
| | | | | QTWTDERLKFGGPTEI | | |
| 789. | GABRA6 | GABRA612 | 66 | MFGPVSDVEMEYTMDVFFRQTWTDE | −3.17 | 5.18E−03 |
| | | | | RLKFGGPTEILSLNNL | | |
| 790. | GABRA6 | GABRA6_34 | 198 | MPEESSSLLQYDLIGQTVSSETIKS | −1.92 | 3.90E−02 |
| | | | | NTGEYVIMTVYFHLQR | | |
| 791. | GABRA6 | GABRA635 | 204 | MLLQYDLIGQTVSSETIKSNTGEYV | −1.96 | 3.54E−02 |
| | | | | IMTVYFHLQRKMGYFM | | |
| 792. | GEN1 | GEN1_32 | 186 | MIKSKLGLDRDALVGLAILLGCDYL | −2.43 | 1.67E−02 |
| | | | | PKGVPGVGKEQALKLI | | |
| 793. | GEN1 | GEN1_33 | 192 | MLDRDALVGLAILLGCDYLPKGVPG | −2.41 | 1.72E−02 |
| | | | | VGKEQALKLIQILKGQ | | |
| 794. | GEN1 | GEN1_34 | 198 | MVGLAILLGCDYLPKGVPGVGKEQA | −2.24 | 1.81E−02 |
| | | | | LKLIQILKGQSLLQRF | | |
| 795. | GEN1 | GEN1_64 | 378 | MLTHYDMIERKLGSRNSNQLQPIRI | −3.16 | 5.31E−03 |
| | | | | VKTRIRNGVHCFEIEW | | |
| 796. | GEN1 | GEN1_65 | 384 | MIERKLGSRNSNQLQPIRIVKTRIR | −3.24 | 4.62E−03 |
| | | | | NGVHCFEIEWEKPEHY | | |
| 797. | GEN1 | GEN1_66 | 390 | MSRNSNQLQPIRIVKTRIRNGVHCF | −2.67 | 1.95E−02 |
| | | | | EIEWEKPEHYAMEDKQ | | |
| 798. | GEN1 | GEN1_67 | 396 | MLQPIRIVKTRIRNGVHCFEIEWEK | −3.43 | 3.29E−03 |
| | | | | PEHYAMEDKQHGEFAL | | |
| 799. | GEN1 | GEN1_68 | 402 | MVKTRIRNGVHCFEIEWEKPEHYAM | −2.38 | 1.79E−02 |
| | | | | EDKQHGEFALLTIEEE | | |
| 800. | GLI1 | GLI1_41 | 240 | MGCSQEFDSQEQLVHHINSEHIHGE | −2.18 | 3.05E−02 |
| | | | | RKEFVCHWGGCSRELR | | |
| 801. | GLI1 | GLI1_42 | 246 | MDSQEQLVHHINSEHIHGERKEFVC | −2.99 | 7.00E−03 |
| | | | | HWGGCSRELRPFKAQY | | |
| 802. | GMPS | GMPS_48 | 282 | MVEEALKKLGIQVKVINAAHSFYNG | −2.09 | 4.76E−02 |
| | | | | TTTLPISDEDRTPRKR | | |
| 803. | GMPS | GMPS_49 | 288 | MKLGIQVKVINAAHSFYNGTTTLPI | −2.18 | 4.32E−02 |
| | | | | SDEDRTPRKRISKTLN | | |
| 804. | GNA13 | GNA13_30 | 174 | MKYFLDNLDKLGEPDYIPSQQDILL | −2.60 | 2.52E−02 |
| | | | | ARRPTKGIHEYDFEIK | | |
| 805. | GNA13 | GNA13_31 | 180 | MLDKLGEPDYIPSQQDILLARRPTK | −2.49 | 3.08E−02 |
| | | | | GIHEYDFEIKNVPFKM | | |
| 806. | GNA13 | GNA13_32 | 186 | MPDYIPSQQDILLARRPTKGIHEYD | −2.73 | 2.01E−02 |
| | | | | FEIKNVPFKMVDVGGQ | | |
| 807. | GNAS | GNAS_4 | 18 | MQREANKKIEKQLQKDKQVYRATHR | −2.22 | 4.06E−02 |
| | | | | LLLLGAGESGKSTIVK | | |
| 808. | GNAS | GNAS_7 | 36 | MYRATHRLLLLGAGESGKSTIVKQM | −2.19 | 2.20E−02 |
| | | | | RILHVNGFNGEGGEED | | |
| 809. | GNAS | GNAS_9 | 48 | MGESGKSTIVKQMRILHVNGFNGEG | −3.03 | 6.53E−03 |
| | | | | GEEDPQAARSNSDGSE | | |
| 810. | GRIN2D | GRIN2D_61 | 360 | MHRYFMNITWDNRDYSFNEDGFLVN | −2.37 | 1.50E−02 |
| | | | | PSLVVISLTRDRTWEV | | |
| 811. | GRIN2D | GRIN2D62 | 366 | MITWDNRDYSFNEDGFLVNPSLVVI | −2.45 | 1.62E−02 |
| | | | | SLTRDRTWEVVGSWEQ | | |
| 812. | GRIN2D | GRIN2D80 | 474 | MDAPRPEKRCCKGFCIDILKRLAHT | −1.95 | 5.95E−02 |
| | | | | IGFSYDLYLVTNGKHG | | |
| 813. | GRIN2D | GRIN2D81 | 480 | MKRCCKGFCIDILKRLAHTIGFSYD | −2.66 | 2.21E−02 |
| | | | | LYLVINGKHGKKIDGV | | |
| 814. | GRIN2D | GRIN2D82 | 486 | MFCIDILKRLAHTIGFSYDLYLVTN | −3.65 | 2.16E−03 |
| | | | | GKHGKKIDGVWNGMIG | | |
| 815. | GRIN2D | GRIN2D95 | 564 | MVARSNGTVSPSAFLEPYSPAVWVM | −3.48 | 4.27E−03 |
| | | | | MFVMCLTVVAVTVFIF | | |
| 816. | GRIN2D | GRIN2D96 | 570 | MTVSPSAFLEPYSPAVWVMMFVMCL | −2.19 | 2.56E−02 |
| | | | | TVVAVTVFIFEYLSPV | | |
| 817. | GRIN2D | GRIN2D97 | 576 | MFLEPYSPAVWVMMFVMCLTVVAVT | −3.26 | 6.10E−03 |
| | | | | VFIFEYLSPVGYNRSL | | |
| 818. | GRIN2D | GRIN2D98 | 582 | MPAVWVMMFVMCLTVVAVTVFIFEY | −3.30 | 5.82E−03 |
| | | | | LSPVGYNRSLATGKRP | | |
| 819. | GTF2\| | GTF21_79 | 468 | MNIPFRSPSWYGIPRLEKIIQVGNR | −2.51 | 2.51E−02 |
| | | | | IKFVIKRPELLTHSTT | | |
| 820. | GTF2 | GTF21_82 | 486 | MIQVGNRIKFVIKRPELLTHSTTEV | −3.16 | 9.12E−03 |
| | | | | TQPRTNTPVKEDWNVR | | |
| 821. | GTF21 | GTF21_83 | 492 | MIKFVIKRPELLTHSTTEVTQPRTN | −3.31 | 6.57E−03 |
| | | | | TPVKEDWNVRITKLRK | | |
| 822. | GTF21 | GTF21_84 | 498 | MRPELLTHSTTEVTQPRTNTPVKED | −3.39 | 4.90E−03 |
| | | | | WNVRITKLRKQVEEIF | | |
| 823. | GTF2\| | GTF21_85 | 504 | MHSTTEVTQPRTNTPVKEDWNVRIT | −1.81 | 7.22E−02 |
| | | | | KLRKQVEEIFNLKFAQ | | |
| 824. | HCK | HCK_74 | 438 | MGMSNPEVIRALERGYRMPRPENCP | −3.45 | 5.17E−03 |
| | | | | EELYNIMMRCWKNRPE | | |
| 825. | HCK | HCK_76 | 450 | MRGYRMPRPENCPEELYNIMMRCWK | −2.51 | 9.35E−03 |
| | | | | NRPEERPTFEYIQSVL | | |
| 826. | HGF | HGF_8 | 42 | MKKSAKTTLIKIDPALKIKTKKVNT | −2.07 | 5.92E−02 |
| | | | | ADQCANRCTRNKGLPF | | |
| 827. | HGF | HGF_9 | 48 | MTLIKIDPALKIKTKKVNTADQCAN | −1.90 | 7.56E−02 |
| | | | | RCTRNKGLPFTCKAFV | | |
| 828. | HGF | HGF_10 | 54 | MPALKIKTKKVNTADQCANRCTRNK | −2.13 | 5.27E−02 |
| | | | | GLPFTCKAFVFDKARK | | |
| 829. | HGF | HGF_11 | 60 | MTKKVNTADQCANRCTRNKGLPFTC | −1.85 | 8.27E−02 |
| | | | | KAFVFDKARKQCLWFP | | |
| 830. | HGF | HGF_68 | 402 | MLSQTRSGLTCSMWDKNMEDLHRHI | −1.89 | 5.53E−02 |
| | | | | FWEPDASKLNENYCRN | | |
| 831. | HGF | HGF_69 | 408 | MGLTCSMWDKNMEDLHRHIFWEPDA | −1.84 | 5.19E−02 |
| | | | | SKLNENYCRNPDDDAH | | |
| 832. | HIF3A | HIF3A_51 | 300 | MTGQYRFLARSGGYLWTQTQATVVS | −2.38 | 2.50E−02 |
| | | | | GGRGPQSESIVCVHFL | | |
| 833. | HIF3A | HIF3A_53 | 312 | MYLWTQTQATVVSGGRGPQSESIVC | −1.76 | 4.09E−02 |
| | | | | VHFLISRVEETGVVLS | | |
| 834. | HIST1H1C | HIST1H1C_15 | 84 | MKSLVSKGTLVQTKGTGASGSFKLN | −1.76 | 6.67E−02 |
| | | | | KKAASGEAKPKVKKAG | | |
| 835. | HIST1H1C | HIST1H1C_16 | 90 | MGTLVQTKGTGASGSFKLNKKAASG | −2.17 | 4.00E−02 |
| | | | | EAKPKVKKAGGTKPKK | | |
| 836. | HIST1H1E | HIST1H1E_22 | 126 | MKAKKPAGAAKKPKKATGAATPKKS | −1.92 | 3.45E−02 |
| | | | | AKKTPKKAKKPAAAAG | | |
| 837. | HIST1H1E | HIST1H1E_24 | 138 | MKKATGAATPKKSAKKTPKKAKKPA | −1.81 | 3.32E−02 |
| | | | | AAAGAKKAKSPKKAKA | | |
| 838. | HIST1H1E | HIST1H1E_25 | 144 | MATPKKSAKKTPKKAKKPAAAAGAK | −1.80 | 4.08E−02 |
| | | | | KAKSPKKAKAAKPKKA | | |
| 839. | HRAS | HRAS_13 | 72 | MRTGEGFLCVFAINNTKSFEDIHQY | −2.26 | 3.19E−03 |
| | | | | REQIKRVKDSDDVPMV | | |
| 840. | HRAS | HRAS_17 | 96 | MREQIKRVKDSDDVPMVLVGNKCDL | −1.97 | 6.76E−02 |
| | | | | AARTVESRQAQDLARS | | |
| 841. | HTATIP2 | HTATIP223 | 132 | MKGADKSSNFLYLQVKGEVEAKVEE | −2.27 | 4.31E−02 |
| | | | | LKFDRYSVFRPGVLLC | | |
| 842. | HTATIP2 | HTATIP224 | 138 | MSNFLYLQVKGEVEAKVEELKFDRY | −3.06 | 9.01E−03 |
| | | | | SVFRPGVLLCDRQESR | | |
| 843. | HTATIP2 | HTATIP225 | 144 | MQVKGEVEAKVEELKFDRYSVFRPG | −2.27 | 4.36E−02 |
| | | | | VLLCDRQESRPGEWLV | | |
| 844. | HUWE1 | HUWE141 | 240 | MESLTKMYSIPKDKQMLLFTHIRLA | −2.01 | 3.06E−02 |
| | | | | HGFSNHRKRLQAVQAR | | |
| 845. | HUWE1 | HUWE145 | 264 | MHGFSNHRKRLQAVQARLHAISILV | −2.79 | 1.24E−02 |
| | | | | YSNALQESANSILYNG | | |
| 846. | HUWE1 | HUWE147 | 276 | MVQARLHAISILVYSNALQESANSI | −1.78 | 4.81E−02 |
| | | | | LYNGLIEELVDVLQIT | | |
| 847. | HUWE1 | HUWE179 | 468 | MKECPFVIKPKIQRPNTTQEGEEME | −3.92 | 1.27E−03 |
| | | | | TDMDGVQCIPQRAALL | | |
| 848. | HUWE1 | HUWE180 | 474 | MIKPKIQRPNTTQEGEEMETDMDGV | −2.44 | 2.65E−02 |
| | | | | QCIPQRAALLKSMLNF | | |
| 849. | HUWE1 | HUWE1381 | 2280 | MGASQDSSSNQQDPGEPGEAEVQEE | −2.90 | 8.13E−03 |
| | | | | DHDVTQTEVADGDIMD | | |
| 850. | HUWE1 | HUWE1382 | 2286 | MSSNQQDPGEPGEAEVQEEDHDVTQ | −2.84 | 8.93E−03 |
| | | | | TEVADGDIMDGEAETD | | |
| 851. | HUWE1 | HUWE1383 | 2292 | MPGEPGEAEVQEEDHDVTQTEVADG | −2.79 | 9.71E−03 |
| | | | | DIMDGEAETDSVVIAG | | |
| 852. | HUWE1 | HUWE1461 | 2760 | MTLGTLQSSQQQPTLPTPPALGEVP | −2.21 | 3.56E−02 |
| | | | | QELQSPAGEGGSSTQL | | |
| 853. | HUWE1 | HUWE1462 | 2766 | MSSQQQPTLPTPPALGEVPQELQSP | −2.30 | 1.35E−02 |
| | | | | AGEGGSSTQLLMPVEP | | |
| 854. | HUWE1 | HUWE1465 | 2784 | MQELQSPAGEGGSSTQLLMPVEPEE | −2.36 | 1.85E−02 |
| | | | | LGPTRPSGEAETTQME | | |
| 855. | HUWE1 | HUWE1589 | 3528 | MAASTTVTTPTTATTTVSISPTTKG | −2.08 | 3.55E−02 |
| | | | | SKSPAKVSDGGSSSTD | | |
| 856. | HUWE1 | HUWE1591 | 3540 | MTTTVSISPTTKGSKSPAKVSDGGS | −2.43 | 3.34E−03 |
| | | | | SSTDFKMVSSGLTENQ | | |
| 857. | HUWE1 | HUWE1593 | 3552 | MSKSPAKVSDGGSSSTDFKMVSSGL | −1.89 | 1.42E−02 |
| | | | | TENQLQLSVEVLTSHS | | |
| 858. | IL6ST | IL6ST_39 | 228 | MSVINSEELSSILKLTWTNPSIKSV | −2.37 | 3.08E−02 |
| | | | | IILKYNIQYRTKDAST | | |
| 859. | IL6ST | IL6ST_40 | 234 | MELSSILKLTWTNPSIKSVIILKYN | −1.88 | 7.47E−02 |
| | | | | IQYRTKDASTWSQIPP | | |
| 860. | IL6ST | IL6ST_41 | 240 | MKLTWTNPSIKSVIILKYNIQYRTK | −2.47 | 1.28E−02 |
| | | | | DASTWSQIPPEDTAST | | |
| 861. | IL6ST | IL6ST_72 | 426 | MVMDLKAFPKDNMLWVEWTTPRESV | −2.84 | 8.93E−03 |
| | | | | KKYILEWCVLSDKAPC | | |
| 862. | IL6ST | IL6ST_73 | 432 | MFPKDNMLWVEWTTPRESVKKYILE | −1.89 | 7.83E−02 |
| | | | | WCVLSDKAPCITDWQQ | | |
| 863. | IL6ST | IL6ST_75 | 444 | MTPRESVKKYILEWCVLSDKAPCIT | −2.96 | 1.15E−02 |
| | | | | DWQQEDGTVHRTYLRG | | |
| 864. | IL6ST | IL6ST_76 | 450 | MKKYILEWCVLSDKAPCITDWQQED | −2.13 | 3.67E−02 |
| | | | | GTVHRTYLRGNLAESK | | |
| 865. | IL6ST | IL6ST_77 | 456 | MWCVLSDKAPCITDWQQEDGTVHRT | −2.73 | 1.67E−02 |
| | | | | YLRGNLAESKCYLITV | | |
| 866. | IL7R | IL7R_15 | 84 | MFRKLQEIYFIETKKFLLIGKSNIC | −2.28 | 2.07E−02 |
| | | | | VKVGEKSLTCKKIDLT | | |
| 867. | IL7R | IL7R_16 | 90 | MIYFIETKKFLLIGKSNICVKVGEK | −2.23 | 2.23E−02 |
| | | | | SLTCKKIDLTTIVKPE | | |
| 868. | IL7R | IL7R_17 | 96 | MKKFLLIGKSNICVKVGEKSLTCKK | −2.20 | 2.33E−02 |
| | | | | IDLTTIVKPEAPFDLS | | |
| 869. | IL7R | IL7R_18 | 102 | MGKSNICVKVGEKSLTCKKIDLTTI | −2.29 | 2.07E−02 |
| | | | | VKPEAPFDLSVVYREG | | |
| 870. | ING1 | ING1_10 | 54 | MYERFSRETDGAQKRRMLHCVQRAL | −1.90 | 6.21E−02 |
| | | | | IRSQELGDEKIQIVSQ | | |
| 871. | ING1 | ING1_14 | 78 | MIRSQELGDEKIQIVSQMVELVENR | −5.40 | 2.83E−05 |
| | | | | TRQVDSHVELFEAQQE | | |
| 872. | ING1 | ING1_16 | 90 | MIVSQMVELVENRTRQVDSHVELFE | −2.06 | 5.18E−03 |
| | | | | AQQELGDTAGNSGKAG | | |
| 873. | ING1 | ING1_17 | 96 | MELVENRTRQVDSHVELFEAQQELG | −4.31 | 5.44E−04 |
| | | | | DTAGNSGKAGADRPKG | | |
| 874. | ING1 | ING1_21 | 120 | MDTAGNSGKAGADRPKGEAAAQADK | −3.34 | 3.84E−03 |
| | | | | PNSKRSRRORNNENRE | | |
| 875. | ING1 | ING1_24 | 138 | MAAQADKPNSKRSRRQRNNENRENA | −3.24 | 4.55E−03 |
| | | | | SSNHDHDDGASGTPKE | | |
| 876. | ING4 | ING4_16 | 90 | MEMVDKHIRRLDTDLARFEADLKEK | −2.90 | 8.17E−03 |
| | | | | QIESSDYDSSSSKGKK | | |
| 877. | ING4 | ING4_22 | 126 | MKGKKKGRTQKEKKAARARSKGKNS | −2.79 | 9.63E−03 |
| | | | | DEEAPKTAQKKLKLVR | | |
| 878. | ING4 | ING4_23 | 132 | MRTQKEKKAARARSKGKNSDEEAPK | −2.67 | 1.16E−02 |
| | | | | TAQKKLKLVRTSPEYG | | |
| 879. | JAK1 | JAK1_143 | 852 | MPDIVSEKKPATEVDPTHFEKRFLK | −2.68 | 2.15E−02 |
| | | | | RIRDLGEGHFGKVELC | | |
| 880. | JAK1 | JAK1_144 | 858 | MKKPATEVDPTHFEKRFLKRIRDLG | −2.03 | 5.41E−02 |
| | | | | EGHFGKVELCRYDPEG | | |
| 881. | JAK1 | JAK1_145 | 864 | MVDPTHFEKRFLKRIRDLGEGHFGK | −4.42 | 4.25E−04 |
| | | | | VELCRYDPEGDNTGEQ | | |
| 882. | JAK1 | JAK1_146 | 870 | MEKRFLKRIRDLGEGHFGKVELCRY | −4.39 | 4.50E−04 |
| | | | | DPEGDNTGEQVAVKSL | | |
| 883. | JAK2 | JAK2_42 | 246 | MLKLKYLINLETLQSAFYTEKFEVK | −2.17 | 2.43E−02 |
| | | | | EPGSGPSGEEIFATII | | |
| 884. | JAK2 | JAK2_43 | 252 | MINLETLQSAFYTEKFEVKEPGSGP | −2.14 | 2.53E−02 |
| | | | | SGEEIFATIIITGNGG | | |
| 885. | JAK2 | JAK2_44 | 258 | MQSAFYTEKFEVKEPGSGPSGEEIF | −2.09 | 2.74E−02 |
| | | | | ATIIITGNGGIQWSRG | | |
| 886. | JAK2 | JAK2_45 | 264 | MEKFEVKEPGSGPSGEEIFATIIIT | −2.19 | 2.37E−02 |
| | | | | GNGGIQWSRGKHKESE | | |
| 887. | JAK2 | JAK2_54 | 318 | MNIIDVSIKQANQEGSNESRVVTIH | −2.17 | 5.07E−02 |
| | | | | KQDGKNLEIELSSLRE | | |
| 888. | JAK2 | JAK2_56 | 330 | MEGSNESRVVTIHKQDGKNLEIELS | −2.06 | 5.92E−02 |
| | | | | SLREALSFVSLIDGYY | | |
| 889. | JAK2 | JAK2_57 | 336 | MRVVTIHKQDGKNLEIELSSLREAL | −2.06 | 5.98E−02 |
| | | | | SFVSLIDGYYRLTADA | | |
| 890. | JAK2 | JAK2_77 | 456 | MEEYNLSGTKKNFSSLKDLLNCYQM | −2.15 | 5.32E−02 |
| | | | | ETVRSDNIIFQFTKCC | | |
| 891. | JAK2 | JAK2_79 | 468 | MSSLKDLLNCYQMETVRSDNIIFQF | −1.78 | 7.21E−02 |
| | | | | TKCCPPKPKDKSNLLV | | |
| 892. | JAK2 | JAK2_155 | 924 | MIMEYLPYGSLRDYLQKHKERIDHI | −1.89 | 7.18E−02 |
| | | | | KLLQYTSQICKGMEYL | | |
| 893. | JAK2 | JAK2_156 | 930 | MYGSLRDYLQKHKERIDHIKLLQYT | −2.06 | 5.69E−02 |
| | | | | SQICKGMEYLGTKRYI | | |
| 894. | JAK3 | JAK3_20 | 114 | MCHRFGLRKDLASAILDLPVLEHLF | −1.89 | 2.86E−03 |
| | | | | AQHRSDLVSGRLPVGL | | |
| 895. | JAK3 | JAK3_23 | 132 | MVLEHLFAQHRSDLVSGRLPVGLSL | −1.91 | 1.15E−03 |
| | | | | KEQGECLSLAVLDLAR | | |
| 896. | JAK3 | JAK3_26 | 150 | MPVGLSLKEQGECLSLAVLDLARMA | −2.32 | 2.96E−02 |
| | | | | REQAQRPGELLKTVSY | | |
| 897. | JAK3 | JAK3_30 | 174 | MREQAQRPGELLKTVSYKACLPPSL | −2.01 | 4.66E−03 |
| | | | | RDLIQGLSFVTRRRIR | | |
| 898. | KANK1 | KANK1_44 | 258 | MHLQHIREQMAIALKRLKELEEQVR | −2.82 | 8.23E−03 |
| | | | | TIPVLQVKISVLQEEK | | |
| 899. | KANK1 | KANK1_45 | 264 | MEQMAIALKRLKELEEQVRTIPVLQ | −3.38 | 4.24E−03 |
| | | | | VKISVLQEEKRQLVSQ | | |
| 900. | KANK1 | KANK1_71 | 420 | MSCKDAAVGTLVQMRNCGVSVTEAM | −2.97 | 8.16E−03 |
| | | | | LGVMTEADKEIELQQQ | | |
| 901. | KANK1 | KANK1_73 | 432 | MMRNCGVSVTEAMLGVMTEADKEIE | −1.89 | 7.68E−02 |
| | | | | LQQQTIESLKEKIYRL | | |
| 902. | KANK1 | KANK1_87 | 516 | MEAVVQTRDQMVGSHMDLVDTCVGT | −2.76 | 2.84E−03 |
| | | | | SVETNSVGISCQPECK | | |
| 903. | KANK1 | KANK1_88 | 522 | MRDQMVGSHMDLVDTCVGTSVETNS | −2.64 | 2.07E−02 |
| | | | | VGISCQPECKNKVVGP | | |
| 904. | KANK1 | KANK1_93 | 552 | MPECKNKVVGPELPMNWWIVKERVE | −2.47 | 1.57E−02 |
| | | | | MHDRCAGRSVEMCDKS | | |
| 905. | KANK1 | KANK1_94 | 558 | MVVGPELPMNWWIVKERVEMHDRCA | −4.69 | 2.19E−04 |
| | | | | GRSVEMCDKSVSVEVS | | |
| 906. | KANK1 | KANK1_96 | 570 | MVKERVEMHDRCAGRSVEMCDKSVS | −1.82 | 6.01E−03 |
| | | | | VEVSVCETGSNTEESV | | |
| 907. | KANK1 | KANK1_115 | 684 | MNTETATLIESCTNTCLSTLDKQTS | −2.32 | 3.24E−02 |
| | | | | TQTVETRTVAVGEGRV | | |
| 908. | KANK1 | KANK1_116 | 690 | MLIESCTNTCLSTLDKQTSTQTVET | −1.96 | 9.68E−03 |
| | | | | RTVAVGEGRVKDINSS | | |
| 909. | KAT6A | KAT6A_71 | 420 | MPDGRKARGEVVDYSEQYRIRKRGN | −4.24 | 6.45E−04 |
| | | | | RKSSTSDWPTDNQDGW | | |
| 910. | KAT6A | KAT6A_74 | 438 | MIRKRGNRKSSTSDWPTDNQDGWDG | −3.20 | 4.93E−03 |
| | | | | KQENEERLFGSQEIMT | | |
| 911. | KAT6A | KAT6A_101 | 600 | MLDHKTLYYDVEPFLFYVLTQNDVK | −2.24 | 4.08E−02 |
| | | | | GCHLVGYFSKEKHCQQ | | |
| 912. | KAT6A | KAT6A_102 | 606 | MYYDVEPFLFYVLTQNDVKGCHLVG | −2.20 | 4.48E−02 |
| | | | | YFSKEKHCQQKYNVSC | | |
| 913. | KAT6A | KAT6A_143 | 852 | MHDSLPANSQPSRRGRWGRKNRKTQ | −1.84 | 8.31E−02 |
| | | | | ERFGDKDSKLLLEETS | | |
| 914. | KAT6A | KAT6A_144 | 858 | MNSQPSRRGRWGRKNRKTQERFGDK | −2.70 | 1.12E−02 |
| | | | | DSKLLLEETSSAPQEQ | | |
| 915. | KAT6A | KAT6A_145 | 864 | MRGRWGRKNRKTQERFGDKDSKLLL | −3.43 | 3.28E−03 |
| | | | | EETSSAPQEQYGECGE | | |
| 916. | KAT6A | KAT6A_146 | 870 | MKNRKTQERFGDKDSKLLLEETSSA | −3.14 | 6.57E−03 |
| | | | | PQEQYGECGEKSEATQ | | |
| 917. | KAT6A | KAT6A_147 | 876 | MERFGDKDSKLLLEETSSAPQEQYG | −2.91 | 6.45E−03 |
| | | | | ECGEKSEATQEQYTES | | |
| 918. | KAT6A | KAT6A_148 | 882 | MDSKLLLEETSSAPQEQYGECGEKS | −2.98 | 7.16E−03 |
| | | | | EATQEQYTESEEQLVA | | |
| 919. | KAT6A | KAT6A_171 | 1020 | MRRVRKRKHHNSSVVTETISETTEV | −3.43 | 3.30E−03 |
| | | | | LDEPFEDSDSERPMPR | | |
| 920. | KAT6A | KAT6A_175 | 1044 | MLDEPFEDSDSERPMPRLEPTFEID | −1.80 | 6.45E−02 |
| | | | | EEEEEEDENELFPREY | | |
| 921. | KAT6A | KAT6A_179 | 1068 | MEEEEEEDENELFPREYFRRLSSQD | −1.92 | 4.32E−02 |
| | | | | VLRCQSSSKRKSKDEE | | |
| 922. | KAT6A | KAT6A_200 | 1194 | MIPKAGRKPKIQESEETVEPKEDMP | −2.04 | 2.90E−02 |
| | | | | LPEERKEEEEMQAEAE | | |
| 923. | KAT6A | KAT6A_201 | 1200 | MKPKIQESEETVEPKEDMPLPEERK | −2.23 | 2.25E−02 |
| | | | | EEEEMQAEAEEAEEGE | | |
| 924. | KAT6A | KAT6A_202 | 1206 | MSEETVEPKEDMPLPEERKEEEEMQ | −2.21 | 4.37E−02 |
| | | | | AEAEEAEEGEEEDAAS | | |
| 925. | KAT6A | KAT6A_210 | 1254 | MADSSNSPETETKEPEVEEEEEKPR | −2.14 | 2.53E−02 |
| | | | | VSEEQRQSEEEQQELE | | |
| 926. | KAT6A | KAT6A_211 | 1260 | MPETETKEPEVEEEEEKPRVSEEQR | −2.30 | 2.02E−02 |
| | | | | QSEEEQQELEEPEPEE | | |
| 927. | KAT6A | KAT6A_213 | 1272 | MEEEKPRVSEEQRQSEEEQQELEEP | −3.37 | 3.62E−03 |
| | | | | EPEEEEDAAAETAQND | | |
| 928. | KAT6A | KAT6A_214 | 1278 | MVSEEQRQSEEEQQELEEPEPEEEE | −2.78 | 9.82E−03 |
| | | | | DAAAETAQNDDHDADD | | |
| 929. | KAT6A | KAT6A_215 | 1284 | MQSEEEQQELEEPEPEEEEDAAAET | −2.69 | 1.14E−02 |
| | | | | AQNDDHDADDEDDGHL | | |
| 930. | KCTD11 | KCTD1127 | 156 | MLEWAPRPVELPEVEYGRLGLQPLW | −3.56 | 2.59E−03 |
| | | | | TGGPGERREVVGTPSF | | |
| 931. | KCTD11 | KCTD1128 | 162 | MPVELPEVEYGRLGLQPLWTGGPGE | −2.47 | 4.45E−03 |
| | | | | RREVVGTPSFLEEVLR | | |
| 932. | KCTD21 | KCTD2130 | 174 | MTSHLQDPNHLTLDWVANVEGLPEE | −3.56 | 1.93E−03 |
| | | | | EYTKQNLKRLWVVPAN | | |
| 933. | KCTD21 | KCTD2133 | 192 | MEGLPEEEYTKQNLKRLWVVPANKQ | −1.80 | 4.06E−02 |
| | | | | INSFQVFVEEVLKIAL | | |
| 934. | KCTD21 | KCTD2134 | 198 | MEYTKQNLKRLWVVPANKQINSFQV | −1.84 | 6.59E−02 |
| | | | | FVEEVLKIALSDGFCI | | |
| 935. | KDM5C | KDM5C_224 | 1338 | MSGKDMPKVQGLLENGDSVTSPEKV | −4.22 | 6.63E−04 |
| | | | | APEEGSGKRDLELLSS | | |
| 936. | KDM5C | KDM5C225 | 1344 | MKVQGLLENGDSVTSPEKVAPEEGS | −4.51 | 3.44E−04 |
| | | | | GKRDLELLSSLLPQLT | | |
| 937. | KDM5C | KDM5C_226 | 1350 | MENGDSVTSPEKVAPEEGSGKRDLE | −2.86 | 7.59E−03 |
| | | | | LLSSLLPQLTGPVLEL | | |
| 938. | KDM5C | KDM5C_227 | 1356 | MTSPEKVAPEEGSGKRDLELLSSLL | −3.54 | 4.69E−04 |
| | | | | PQLTGPVLELPEATRA | | |
| 939. | KDM6A | KDM6A4 | 18 | MGDEEKKMAAGKASGESEEASPSLT | −1.96 | 3.27E−02 |
| | | | | AEEREALGGLDSRLFG | | |
| 940. | KDM6A | KDM6A5 | 24 | MMAAGKASGESEEASPSLTAEEREA | −1.91 | 7.55E−02 |
| | | | | LGGLDSRLFGFVRFHE | | |
| 941. | KDM6A | KDM6A161 | 960 | MTNPNNPVTVIRGLAGALKLDLGLF | −2.60 | 1.96E−02 |
| | | | | STKTLVEANNEHMVEV | | |
| 942. | KDM6A | KDM6A162 | 966 | MVTVIRGLAGALKLDLGLFSTKTLV | −3.10 | 8.99E−03 |
| | | | | EANNEHMVEVRTQLLQ | | |
| 943. | KDM6A | KDM6A193 | 1152 | MCSVNINIGPGDCEWFVVPEGYWGV | −3.25 | 3.55E−03 |
| | | | | LNDFCEKNNLNFLMGS | | |
| 944. | KDM6A | KDM6A194 | 1158 | MIGPGDCEWFVVPEGYWGVLNDFCE | −3.21 | 8.46E−03 |
| | | | | KNNLNFLMGSWWPNLE | | |
| 945. | KDM6A | KDM6A195 | 1164 | MEWFWPEGYWGVLNDFCEKNNLNFL | −2.57 | 2.34E−02 |
| | | | | MGSWWPNLEDLYEAN | | |
| 946. | KDM6A | KDM6A196 | 1170 | MEGYWGVLNDFCEKNNLNFLMGSWW | −2.45 | 2.82E−02 |
| | | | | PNLEDLYEANVPVYRF | | |
| 947. | KDM6A | KDM6A197 | 1176 | MLNDFCEKNNLNFLMGSWWPNLEDL | −2.07 | 4.54E−02 |
| | | | | YEANVPVYRFIQRPGD | | |
| 948. | KDM6A | KDM6A214 | 1278 | MRNIKVSDPKLFEMIKYCLLRTLKQ | −2.98 | 7.12E−03 |
| | | | | CQTLREALIAAGKEII | | |
| 949. | KDM6A | KDM6A215 | 1284 | MDPKLFEMIKYCLLRTLKQCQTLRE | −2.51 | 2.48E−02 |
| | | | | ALIAAGKEIIWHGRTK | | |
| 950. | KDM6A | KDM6A216 | 1290 | MMIKYCLLRTLKQCQTLREALIAAG | −2.38 | 3.65E−02 |
| | | | | KEIIWHGRTKEEPAHY | | |
| 951. | KDM6A | KDM6A217 | 1296 | MLRTLKQCQTLREALIAAGKEIIWH | −3.07 | 6.09E−03 |
| | | | | GRTKEEPAHYCSICEV | | |
| 952. | KDSR | KDSR_9 | 48 | MAIECYKQGAFITLVARNEDKLLQA | −2.05 | 2.87E−02 |
| | | | | KKEIEMHSINDKQVVL | | |
| 953. | KDSR | KDSR_10 | 54 | MQGAFITLVARNEDKLLQAKKEIEM | −2.05 | 2.87E−02 |
| | | | | HSINDKQVVLCISVDV | | |
| 954. | KDSR | KDSR_12 | 66 | MDKLLQAKKEIEMHSINDKQVVLCI | −1.88 | 3.63E−02 |
| | | | | SVDVSQDYNQVENVIK | | |
| 955. | KIF1A | KIF1A_93 | 552 | MAVVTLEPCEGADTYVNGKKVTEPS | −3.86 | 1.45E−03 |
| | | | | ILRSGNRIIMGKSHVF | | |
| 956. | KIF1A | KIF1A_95 | 564 | MTYVNGKKVTEPSILRSGNRIIMGK | −4.16 | 1.02E−03 |
| | | | | SHVFRFNHPEQARQER | | |
| 957. | KIF1A | KIF1A_96 | 570 | MKVTEPSILRSGNRIIMGKSHVFRF | −2.23 | 1.31E−02 |
| | | | | NHPEQARQERERTPCA | | |
| 958. | KIT | KIT_2 | 6 | MAWDFLCVLLLLLRVQTGSSQPSVS | −2.21 | 4.14E−02 |
| | | | | PGEPSPPSIHPGKSDL | | |
| 959. | KIT | KIT_3 | 12 | MVLLLLLRVQTGSSQPSVSPGEPSP | −2.33 | 3.32E−02 |
| | | | | PSIHPGKSDLIVRVGD | | |
| 960. | KIT | KIT_84 | 498 | MKTSAYFNFAFKGNNKEQIHPHTLF | −3.23 | 5.74E−03 |
| | | | | TPLLIGFVIVAGMMCI | | |
| 961. | KIT | KIT_85 | 504 | MNFAFKGNNKEQIHPHTLFTPLLIG | −2.46 | 6.01E−03 |
| | | | | FVIVAGMMCIIVMILT | | |
| 962. | KIT | KIT_86 | 510 | MNNKEQIHPHTLFTPLLIGFVIVAG | −2.78 | 1.71E−02 |
| | | | | MMCIIVMILTYKYLQK | | |
| 963. | KLF5 | KLF5_48 | 282 | MPCTYTMPSQFLPQQATYFPPSPPS | −3.28 | 7.03E−03 |
| | | | | SEPGSPDRQAEMLQNL | | |
| 964. | KLF5 | KLF5_49 | 288 | MPSQFLPQQATYFPPSPPSSEPGSP | −3.24 | 7.55E−03 |
| | | | | DRQAEMLQNLTPPPSY | | |
| 965. | KMT2A | KMT2A_155 | 924 | MSSAKKATGRKKSSSHDSGTDITSV | −2.44 | 3.32E−02 |
| | | | | TLGDTTAVKTKILIKK | | |
| 966. | KMT2A | KMT2A_156 | 930 | MTGRKKSSSHDSGTDITSVTLGDTT | −2.53 | 2.88E−02 |
| | | | | AVKTKILIKKGRGNLE | | |
| 967. | KMT2A | KMT2A_157 | 936 | MSSHDSGTDITSVTLGDTTAVKTKI | −2.21 | 4.70E−02 |
| | | | | LIKKGRGNLEKTNLDL | | |
| 968. | KMT2A | KMT2A_158 | 942 | MTDITSVTLGDTTAVKTKILIKKGR | −1.91 | 6.99E−02 |
| | | | | GNLEKTNLDLGPTAPS | | |
| 969. | KMT2A | KMT2A_633 | 3792 | MPNDEEEEEVOLKSARRATSMDLPM | −2.84 | 8.98E−03 |
| | | | | PMRFRHLKKTSKEAVG | | |
| 970. | KMT2A | KMT2A_634 | 3798 | MEEVQLKSARRATSMDLPMPMRFRH | −3.32 | 3.97E−03 |
| | | | | LKKTSKEAVGVYRSPI | | |
| 971. | KMT2B | KMT2B_131 | 780 | MGSLPLSGVEEKMFSLLKRAKVQLF | −1.81 | 3.28E−02 |
| | | | | KIDQQQQQKVAASMPL | | |
| 972. | KMT2B | KMT2B_133 | 792 | MFSLLKRAKVQLFKIDQQQQQKVAA | −2.82 | 9.15E−03 |
| | | | | SMPLSPGGQMEEVAGA | | |
| 973. | KMT2B | KMT2B_247 | 1476 | MRHSEEGETPDRRAGGQMKGLLLKL | −4.27 | 5.95E−04 |
| | | | | LESAFGWFDAHDPKYW | | |
| 974. | KMT2B | KMT2B_248 | 1482 | METPDRRAGGQMKGLLLKLLESAFG | −1.98 | 5.18E−02 |
| | | | | WFDAHDPKYWRRSTRL | | |
| 975. | KMT2C | KMT2C_83 | 492 | MHLECDKPTDHELDTQLKEEYICMY | −2.16 | 2.77E−02 |
| | | | | CKHLGAEMDRLQPGEE | | |
| 976. | KMT2C | KMT2C_84 | 498 | MPTDHELDTQLKEEYICMYCKHLGA | −1.96 | 4.96E−02 |
| | | | | EMDRLQPGEEVEIAEL | | |
| 977. | KMT2C | KMT2C_88 | 522 | MEMDRLQPGEEVEIAELTTDYNNEM | −2.94 | 7.58E−03 |
| | | | | EVEGPEDQMVFSEQAA | | |
| 978. | KMT2C | KMT2C_286 | 1710 | MMSNDSMKRQQQQDSIDPSSRIDSE | −2.92 | 1.28E−02 |
| | | | | LFKDPLKQRESEHEQE | | |
| 979. | KMT2C | KMT2C_289 | 1728 | MSRIDSELFKDPLKQRESEHEQEWK | −1.82 | 5.97E−02 |
| | | | | FRQQMRQKSKQQAKIE | | |
| 980. | KMT2C | KMT2C_291 | 1740 | MKQRESEHEQEWKFRQQMRQKSKQQ | −1.83 | 6.04E−02 |
| | | | | AKIEATQKLEQVKNEQ | | |
| 981. | KMT2C | KMT2C_415 | 2484 | MPGGSHGTMPSQERFLVPPQQIQGS | −2.67 | 1.17E−02 |
| | | | | GVSPQLRRSVSVDMPR | | |
| 982. | KMT2C | KMT2C_416 | 2490 | MTMPSQERFLVPPQQIQGSGVSPQL | −1.99 | 6.58E−02 |
| | | | | RRSVSVDMPRPLNNSQ | | |
| 983. | KMT2C | KMT2C_417 | 2496 | MRFLVPPQQIQGSGVSPQLRRSVSV | −1.85 | 8.23E−02 |
| | | | | DMPRPLNNSQMNNPVG | | |
| 984. | KMT2C | KMT2C_564 | 3378 | MPPPRVEFDDNNPFSESFQERERKE | −2.03 | 2.95E−02 |
| | | | | RLREQQERQRIQLMQE | | |
| 985. | KMT2C | KMT2C_567 | 3396 | MERERKERLREQQERQRIQLMQEVD | −3.72 | 1.89E−03 |
| | | | | RQRALQQRMEMEQHGM | | |
| 986. | KMT2C | KMT2C_568 | 3402 | MRLREQQERQRIQLMQEVDRQRALQ | −3.87 | 1.40E−03 |
| | | | | QRMEMEQHGMVGSEIS | | |
| 987. | KMT2C | KMT2C_569 | 3408 | MERQRIQLMQEVDRQRALQQRMEME | −4.40 | 4.48E−04 |
| | | | | QHGMVGSEISSSRTSV | | |
| 988. | KMT2D | KMT2D_508 | 3042 | MNGDEFDLLAYTDPELDTGDKKDIF | −2.40 | 1.77E−02 |
| | | | | NEHLRLVESANEKAER | | |
| 989. | KMT2D | KMT2D_509 | 3048 | MLLAYTDPELDTGDKKDIFNEHLRL | −2.19 | 2.34E−02 |
| | | | | VESANEKAEREALLRG | | |
| 990. | KMT2D | KMT2D_900 | 5394 | MWKNNVYLARSRIQGLGLYAAKDLE | −3.03 | 1.07E−02 |
| | | | | KHTMVIEYIGTIIRNE | | |
| 991. | KMT2D | KMT2D_902 | 5406 | MQGLGLYAAKDLEKHTMVIEYIGTI | −3.89 | 1.35E−03 |
| | | | | IRNEVANRREKIYEEQ | | |
| 992. | KMT2D | KMT2D_903 | 5412 | MAAKDLEKHTMVIEYIGTIIRNEVA | −2.10 | 3.94E−02 |
| | | | | NRREKIYEEQNRGIYM | | |
| 993. | KMT2D | KMT2D_908 | 5442 | MYEEQNRGIYMFRINNEHVIDATLT | −1.83 | 2.83E−03 |
| | | | | GGPARYINHSCAPNCV | | |
| 994. | KMT2D | KMT2D_910 | 5454 | MINNEHVIDATLTGGPARYINHSCA | −1.94 | 5.06E−02 |
| | | | | PNCVAEVVTFDKEDKI | | |
| 995. | LACTB | LACTB_18 | 102 | MCFARAIESSRDLLHRIKDEVGAPG | −3.10 | 5.84E−03 |
| | | | | IVVGVSVDGKEVWSEG | | |
| 996. | LACTB | LACTB_19 | 108 | MESSRDLLHRIKDEVGAPGIVVGVS | −3.42 | 3.36E−03 |
| | | | | VDGKEVWSEGLGYADV | | |
| 997. | LACTB | LACTB_20 | 114 | MLHRIKDEVGAPGIVVGVSVDGKEV | −2.96 | 1.18E−02 |
| | | | | WSEGLGYADVENRVPC | | |
| 998. | LATS1 | LATS1_21 | 120 | MQALQKTNNRSIEAAIEFISKMSYQ | −1.98 | 2.63E−02 |
| | | | | DPRREQMAAAAARPIN | | |
| 999. | LATS1 | LATS1_22 | 126 | MNNRSIEAAIEFISKMSYQDPRREQ | −2.30 | 2.01E−02 |
| | | | | MAAAAARPINASMKPG | | |
| 1000. | LATS1 | LATS1_30 | 174 | MKQSWKGSKESLVPQRHGPPLGESV | −3.16 | 5.31E−03 |
| | | | | AYHSESPNSQTDVGRP | | |
| 1001. | LATS1 | LATS1_31 | 180 | MSKESLVPQRHGPPLGESVAYHSES | −2.21 | 4.84E−02 |
| | | | | PNSQTDVGRPLSGSGI | | |
| 1002. | LATS2 | LATS2_88 | 522 | MLLRSKSEQYDLDSLCAGMEQSLRA | −2.27 | 4.34E−02 |
| | | | | GPNEPEGGDKSRKSAK | | |
| 1003. | LATS2 | LATS2_89 | 528 | MEQYDLDSLCAGMEQSLRAGPNEPE | −2.16 | 5.16E−02 |
| | | | | GGDKSRKSAKGDKGGK | | |
| 1004. | LATS2 | LATS2_112 | 666 | MMFVKIKTLGIGAFGEVCLACKVDT | −3.47 | 2.35E−03 |
| | | | | HALYAMKTLRKKDVLN | | |
| 1005. | ATS2 | LATS2_113 | 672 | MTLGIGAFGEVCLACKVDTHALYAM | −4.04 | 9.80E−04 |
| | | | | KTLRKKDVLNRNQVAH | | |
| 1006. | LIN9 | LIN9_82 | 486 | MLLQIKCLAEGGDLNSFEFKSLTDS | −1.94 | 6.75E−02 |
| | | | | LNDIKSTIDASNISCF | | |
| 1007. | LIN9 | LIN9_83 | 492 | MLAEGGDLNSFEFKSLTDSLNDIKS | −2.20 | 4.64E−02 |
| | | | | TIDASNISCFQNNVEI | | |
| 1008. | LIN9 | LIN9_84 | 498 | MLNSFEFKSLTDSLNDIKSTIDASN | −2.31 | 4.01E−02 |
| | | | | ISCFQNNVEIHVAHIQ | | |
| 1009. | LMO2 | LM02_11 | 60 | MGCRLGEVGRRLYYKLGRKLCRRDY | −3.94 | 1.21E−03 |
| | | | | LRLFGQDGLCASCDKR | | |
| 1010. | LYN | LYN_30 | 174 | MVHGDVIKHYKIRSLDNGGYYISPR | −1.95 | 6.71E−02 |
| | | | | ITFPCISDMIKHYQKQ | | |
| 1011. | LYN | LYN_31 | 180 | MKHYKIRSLDNGGYYISPRITFPCI | −1.89 | 7.28E−02 |
| | | | | SDMIKHYQKQADGLCR | | |
| 1012. | MAF | MAF_31 | 180 | MYHHHHHHAAGHHHHPTAGAPGAAG | −2.66 | 1.18E−02 |
| | | | | SAAASAGGAGGAGGGG | | |
| 1013. | MAF | MAF_32 | 186 | MHAAGHHHHPTAGAPGAAGSAAASA | −2.60 | 1.05E−02 |
| | | | | GGAGGAGGGGPASAGG | | |
| 1014. | MAP2K1 | MAP2K152 | 306 | MPMAIFELLDYIVNEPPPKLPSGVF | −1.97 | 4.83E−02 |
| | | | | SLEFQDFVNKCLIKNP | | |
| 1015. | MAP2K1 | MAP2K153 | 312 | MLLDYIVNEPPPKLPSGVFSLEFQD | −1.92 | 5.04E−02 |
| | | | | FVNKCLIKNPAERADL | | |
| 1016. | MAP3K1 | MAP3K146 | 270 | MRKRVSPVPFQSGRITPPRRAPSPD | −3.05 | 6.40E−03 |
| | | | | GFSPYSPEETNRRVNK | | |
| 1017. | MAP3K1 | MAP3K149 | 288 | MRAPSPDGFSPYSPEETNRRVNKVM | −2.32 | 2.43E−02 |
| | | | | RARLYLLQQIGPNSFL | | |
| 1018. | MAP3K1 | MAP3K1106 | 630 | MVVEACCSVLSMVCADPVYKVYVAA | −1.91 | 5.21E−02 |
| | | | | LKTLRAMLVYTPCHSL | | |
| 1019. | MAP3K1 | MAP3K1108 | 642 | MCADPVYKVYVAALKTLRAMLVYTP | −1.94 | 3.34E−02 |
| | | | | CHSLAERIKLQRLLQP | | |
| 1020. | MAP3K1 | MAP3K1200 | 1194 | MMSASQDALPIVPQLQVENGEDIII | −2.79 | 9.71E−03 |
| | | | | IQQDTPETLPGHTKAK | | |
| 1021. | MAP3K1 | MAP3K1201 | 1200 | MALPIVPQLQVENGEDIIIIQQDTP | −3.31 | 4.09E−03 |
| | | | | ETLPGHTKAKQPYRED | | |
| 1022. | MAP3K1 | MAP3K1202 | 1206 | MQLQVENGEDIIIIQQDTPETLPGH | −3.24 | 4.61E−03 |
| | | | | TKAKQPYREDTEWLKG | | |
| 1023. | MAP3K4 | MAP3K471 | 420 | MDIGWPVFEIPSPRPSKGNEPEYEG | −3.26 | 4.41E−03 |
| | | | | DDTEGELKELESSTDE | | |
| 1024. | MAP3K4 | MAP3K472 | 426 | MFEIPSPRPSKGNEPEYEGDDTEGE | −3.24 | 4.57E−03 |
| | | | | LKELESSTDESEEEQI | | |
| 1025. | MAP3K1 | MAP3K473 | 432 | MRPSKGNEPEYEGDDTEGELKELES | −1.87 | 7.11E−02 |
| | | | | STDESEEEQISDPRVP | | |
| 1026. | MAPK1 | MAPK1_41 | 240 | MLGILGSPSQEDLNCIINLKARNYL | −2.18 | 1.97E−02 |
| | | | | LSLPHKNKVPWNRLFP | | |
| 1027. | MAPK1 | MAPK1_43 | 252 | MNCIINLKARNYLLSLPHKNKVPWN | −1.86 | 5.71E−02 |
| | | | | RLFPNADSKALDLLDK | | |
| 1028. | MAS1 | MAS1_47 | 276 | MPFIYFFVGSSKKKRFKESLKVVLT | −1.86 | 7.81E−02 |
| | | | | RAFKDEMQPRROKDNC | | |
| 1029. | MAS1 | MAS1_48 | 282 | MVGSSKKKRFKESLKVVLTRAFKDE | −2.23 | 4.09E−02 |
| | | | | MQPRRQKDNCNTVTVE | | |
| 1030. | MCC | MCC_2 | 6 | MMKYGNDSSAELSELHSAALASLKG | −1.92 | 7.48E02 |
| | | | | DIVELNKRLQQTERER | | |
| 1031. | MCC | MCC_3 | 12 | MSSAELSELHSAALASLKGDIVELN | −2.11 | 5.65E−02 |
| | | | | KRLQQTERERDLLEKK | | |
| 1032. | MCC | MCC_26 | 150 | MDMDNCSDLNSELQRVLTGLENWCG | −4.30 | 5.64E−04 |
| | | | | RKKSSCSLSVAEVDR | | |
| 1033. | MCC | MCC_28 | 162 | MQRVLTGLENVVCGRKKSSCSLSVA | −2.70 | 1.11E−02 |
| | | | | EVDRHIEQLTTASEHC | | |
| 1034. | MCC | MCC_30 | 174 | MGRKKSSCSLSVAEVDRHIEQLTTA | −2.16 | 5.05E−03 |
| | | | | SEHCDLAIKTVEEIEG | | |
| 1035. | MCC | MCC_117 | 696 | MCADAASPALSLAELRTTCSENELA | −2.30 | 2.37E−02 |
| | | | | AEFTNAIRREKKLKAR | | |
| 1036. | MCC | MCC_118 | 702 | MPALSLAELRTTCSENELAAEFTNA | −2.63 | 1.41E−02 |
| | | | | IRREKKLKARVQELVS | | |
| 1037. | MCF2 | MCF2_44 | 258 | MMETAFDGFWEKHQLKMEQYLQLWK | −2.09 | 2.70E−02 |
| | | | | FEQDFQQLVTEVEFLL | | |
| 1038. | MCF2 | MCF2_45 | 264 | MGFWEKHQLKMEQYLQLWKFEQDFQ | −1.91 | 6.40E−02 |
| | | | | QLVTEVEFLLNQQAEL | | |
| 1039. | MCF2 | MCF2_46 | 270 | MQLKMEQYLQLWKFEQDFQQLVTEV | −1.90 | 7.25E−02 |
| | | | | EFLLNQQAELADVTGT | | |
| 1040. | MCF2 | MCF2_105 | 624 | MENCAHAPERVGPCFLERKDDFQMY | −2.33 | 1.93E−02 |
| | | | | AKYCQNKPRSETIWRK | | |
| 1041. | MCF2 | MCF2_107 | 636 | MCFLERKDDFQMYAKYCONKPRSET | −2.36 | 1.86E−02 |
| | | | | IWRKYSECAFFQECQR | | |
| 1042. | MCF2 | MCF2_135 | 804 | MGSDRYPSYSFKHCWKMDEVGITEY | −3.31 | 4.09E−03 |
| | | | | VKGDNRKFEIWYGEKE | | |
| 1043. | MCF2 | MCF2_136 | 810 | MSYSFKHCWKMDEVGITEYVKGDNR | −3.28 | 4.29E−03 |
| | | | | KFEIWYGEKEEVYIVQ | | |
| 1044. | MCF2 | MCF2_147 | 876 | MVKKRKQQDQLTERDKFQISLQQND | −3.46 | 3.12E−03 |
| | | | | EKQQGAFISTEETELE | | |
| 1045. | MCF2 | MCF2_148 | 882 | MQDQLTERDKFQISLQQNDEKQQGA | −2.26 | 4.02E−02 |
| | | | | FISTEETELEHTSTVV | | |
| 1046. | MDM2 | MDM2_8 | 42 | MLLKSVGAQKDTYTMKEVLFYLGQY | −1.86 | 4.68E−02 |
| | | | | IMTKRLYDEKQQHIVY | | |
| 1047. | MDM2 | MDM2_10 | 54 | MTMKEVLFYLGQYIMTKRLYDEKQQ | −1.98 | 6.84E−02 |
| | | | | HIVYCSNDLLGDLFGV | | |
| 1048. | MDM2 | MDM2_11 | 60 | MFYLGQYIMTKRLYDEKQQHIVYCS | −2.37 | 3.64E−02 |
| | | | | NDLLGDLFGVPSFSVK | | |
| 1049. | MDM2 | MDM2_34 | 198 | MFDESLALCVIREICCERSSSSEST | −3.52 | 2.79E−03 |
| | | | | GTPSNPDLDAGVSEHS | | |
| 1050. | MDM2 | MDM2_36 | 210 | MICCERSSSSESTGTPSNPDLDAGV | −3.71 | 1.93E−03 |
| | | | | SEHSGDWLDQDSVSDQ | | |
| 1051. | MDM4 | MDM4_3 | 12 | MSDSACRISPGQINQVRPKLPLLKI | −3.17 | 6.61E−03 |
| | | | | LHAAGAQGEMFTVKEV | | |
| 1052. | MDM4 | MDM4_4 | 18 | MISPGQINQVRPKLPLLKILHAAGA | −2.23 | 2.25E−02 |
| | | | | QGEMFTVKEVMHYLGQ | | |
| 1053. | MDM4 | MDM4_5 | 24 | MNQVRPKLPLLKILHAAGAQGEMFT | −3.77 | 1.73E−03 |
| | | | | VKEVMHYLGQYIMVKQ | | |
| 1054. | MDM4 | MDM4_9 | 48 | MVKEVMHYLGQYIMVKQLYDQQEQH | −2.62 | 1.25E−02 |
| | | | | MVYCGGDLLGELLGRQ | | |
| 1055. | MDM4 | MDM4_63 | 372 | MPKDAYIKKENSKLFDPCNSVEFLD | −2.71 | 8.83E−03 |
| | | | | LAHSSESQETISSMGE | | |
| 1056. | MDM4 | MDM4_64 | 378 | MKKENSKLFDPCNSVEFLDLAHSSE | −2.34 | 3.73E−02 |
| | | | | SQETISSMGEQLDNLS | | |
| 1057. | MECOM | MECOM_19 | 108 | MYVGEQRSNLKDPSYGWEVHLPRSR | −2.43 | 1.67E−02 |
| | | | | RVSVHSWLYLGKRSSD | | |
| 1058. | MECOM | MECOM_20 | 114 | MSNLKDPSYGWEVHLPRSRRVSVHS | −2.14 | 2.55E−02 |
| | | | | WLYLGKRSSDVGIAFS | | |
| 1059. | MED12 | MED12_128 | 762 | MQRDDARHAIKKITKDILKVLNRKG | −2.40 | 1.88E−02 |
| | | | | TAETDQLAPIVPLNPG | | |
| 1060. | MED12 | MED12_129 | 768 | MHAIKKITKDILKVLNRKGTAETDQ | −2.05 | 3.73E−02 |
| | | | | LAPIVPLNPGDLTFLG | | |
| 1061. | MED12 | MED12_201 | 1200 | MSSCDRHLLAASQNRIVDGAVFAVL | −4.09 | 8.85E−04 |
| | | | | KAVFVLGDAELKGSGF | | |
| 1062. | MED12 | MED12_202 | 1206 | MLLAASQNRIVDGAVFAVLKAVFVL | −3.32 | 6.09E−03 |
| | | | | GDAELKGSGFTVTGGT | | |
| 1063. | MED12 | MED12_204 | 1218 | MAVFAVLKAVFVLGDAELKGSGFTV | −3.67 | 2.10E−03 |
| | | | | TGGTEELPEEEGGGGS | | |
| 1064. | MED12 | MED12_208 | 1242 | MTGGTEELPEEEGGGGSGGRRQGGR | −2.73 | 1.61E−02 |
| | | | | NISVETASLDVYAKYV | | |
| 1065. | MED12 | MED12_211 | 1260 | MRRQGGRNISVETASLDVYAKYVLR | −4.30 | 4.08E−04 |
| | | | | SICQQEWVGERCLKSL | | |
| 1066. | MED12 | MED12_227 | 1356 | MRQSSLELQLMIKQTPNNEMNSLLE | −1.78 | 6.50E−02 |
| | | | | NIAKATIEVFQQSAET | | |
| 1067. | MED12 | MED12_230 | 1374 | MMNSLLENIAKATIEVFQQSAETGS | −2.88 | 1.48E−02 |
| | | | | SSGSTASNMPSSSKTK | | |
| 1068. | MEN1 | MEN1_32 | 186 | MYLKGSYMRCDRKMEVAFMVCAINP | −4.82 | 1.57E−04 |
| | | | | SIDLHTDSLELLQLQQ | | |
| 1069. | MEN1 | MEN1_35 | 204 | MVCAINPSIDLHTDSLELLQLQQKL | −1.93 | 1.92E−02 |
| | | | | LWLLYDLGHLERYPMA | | |
| 1070. | MEN1 | MEN1_37 | 216 | MDSLELLQLQQKLLWLLYDLGHLER | −2.63 | 2.44E−02 |
| | | | | YPMALGNLADLEELEP | | |
| 1071. | MEN1 | MEN1_56 | 330 | MEVANDVIPNLLKEAASLLEAGEER | −2.02 | 4.47E−02 |
| | | | | PGEQSQGTQSQGSALQ | | |
| 1072. | MEN1 | MEN1_59 | 348 | MEAGEERPGEQSQGTQSQGSALQDP | −3.34 | 3.86E−03 |
| | | | | ECFAHLLRFYDGICKW | | |
| 1073. | MEN1 | MEN1_76 | 450 | MESKPEEPPPPKKPALDKGLGTGQG | −2.45 | 2.70E−02 |
| | | | | AVSGPPRKPPGTVAGT | | |
| 1074. | MEN1 | MEN1_79 | 468 | MLGTGQGAVSGPPRKPPGTVAGTAR | −2.03 | 3.05E−03 |
| | | | | GPEGGSTAQVPAPAAS | | |
| 1075. | MET | MET_38 | 222 | MKDGFMFLTDQSYIDVLPEFRDSYP | −2.66 | 1.98E−02 |
| | | | | IKYVHAFESNNFIYFL | | |
| 1076. | MET | MET_39 | 228 | MLTDQSYIDVLPEFRDSYPIKYVHA | −2.20 | 3.57E−02 |
| | | | | FESNNFIYFLTVQRET | | |
| 1077. | MET | MET_40 | 234 | MIDVLPEFRDSYPIKYVHAFESNNF | −1.83 | 8.60E−02 |
| | | | | IYFLTVQRETLDAQTF | | |
| 1078. | MET | MET_42 | 246 | MIKYVHAFESNNFIYFLTVQRETLD | −2.28 | 3.55E−02 |
| | | | | AQTFHTRIIRFCSINS | | |
| 1079. | MET | MET_43 | 252 | MFESNNFIYFLTVQRETLDAQTFHT | −2.98 | 1.28E−02 |
| | | | | RIIRFCSINSGLHSYM | | |
| 1080. | MET | MET_45 | 264 | MQRETLDAQTFHTRIIRFCSINSGL | −2.52 | 2.48E−02 |
| | | | | HSYMEMPLECILTEKR | | |
| 1081. | MET | MET_46 | 270 | MAQTFHTRIIRFCSINSGLHSYMEM | −2.77 | 1.88E−02 |
| | | | | PLECILTEKRKKRSTK | | |
| 1082. | MET | MET_126 | 750 | MKSFISGGSTITGVGKNLNSVSVPR | −2.13 | 5.00E−02 |
| | | | | MVINVHEAGRNFTVAC | | |
| 1083. | MET | MET_127 | 756 | MGSTITGVGKNLNSVSVPRMVINVH | −1.86 | 7.04E−02 |
| | | | | EAGRNFTVACQHRSNS | | |
| 1084. | MET | MET_138 | 822 | MLDGILSKYFDLIYVHNPVFKPFEK | −3.85 | 1.48E−03 |
| | | | | PVMISMGNENVLEIKG | | |
| 1085. | MET | MET_139 | 828 | MKYFDLIYVHNPVFKPFEKPVMISM | −3.81 | 1.58E−03 |
| | | | | GNENVLEIKGNDIDPE | | |
| 1086. | MET | MET_140 | 834 | MYVHNPVFKPFEKPVMISMGNENVL | −3.89 | 1.35E−03 |
| | | | | EIKGNDIDPEAVKGEV | | |
| 1087. | MET | MET_180 | 1074 | MSLIVHFNEVIGRGHFGCVYHGTLL | −2.45 | 3.19E−02 |
| | | | | DNDGKKIHCAVKSLNR | | |
| 1088. | MET | MET_183 | 1092 | MYHGTLLDNDGKKIHCAVKSLNRIT | −3.33 | 6.59E−03 |
| | | | | DIGEVSQFLTEGIIMK | | |
| 1089. | MET | MET_184 | 1098 | MDNDGKKIHCAVKSLNRITDIGEVS | −1.88 | 5.81E−02 |
| | | | | QFLTEGIIMKDFSHPN | | |
| 1090. | MET | MET_193 | 1152 | MPLVVLPYMKHGDLRNFIRNETHNP | −2.11 | 4.29E−02 |
| | | | | TVKDLIGFGLQVAKGM | | |
| 1091. | MET | MET_194 | 1158 | MYMKHGDLRNFIRNETHNPTVKDLI | −2.03 | 4.73E−02 |
| | | | | GFGLQVAKGMKYLASK | | |
| 1092. | MFHAS1 | MFHAS191 | 540 | MLCGERELEEKCLDIHRQIALQEKH | −2.26 | 8.51E−03 |
| | | | | DAEGLSRLAKVVDEAL | | |
| 1093. | MFHAS1 | MFHAS192 | 546 | MLEEKCLDIHRQIALQEKHDAEGLS | −4.58 | 2.86E−04 |
| | | | | RLAKWDEALARDFEL | | |
| 1094. | MGA | MGA_188 | 1122 | MEGIREEEEQLKEKKKRKKLEYTIC | −1.99 | 6.55E−02 |
| | | | | ETEPEQPVRHYPLWVK | | |
| 1095. | MGA | MGA_189 | 1128 | MEEQLKEKKKRKKLEYTICETEPEQ | −3.30 | 4.15E−03 |
| | | | | PVRHYPLWVKVEGEVD | | |
| 1096. | MGA | MGA_190 | 1134 | MKKKRKKLEYTICETEPEQPVRHYP | −2.50 | 2.25E−02 |
| | | | | LWVKVEGEVDPEPVYI | | |
| 1097. | MGA | MGA_191 | 1140 | MLEYTICETEPEQPVRHYPLWVKVE | −1.83 | 5.59E−02 |
| | | | | GEVDPEPVYIPTPSVI | | |
| 1098. | MGA | MGA_315 | 1884 | MSLLSSGASFVSQAGTLTLRISPPE | −2.60 | 2.54E−02 |
| | | | | PQSFASKTGSETKITY | | |
| 1099. | MGA | MGA_316 | 1890 | MASFVSQAGTLTLRISPPEPQSFAS | −2.36 | 3.70E−02 |
| | | | | KTGSETKITYSSGGQP | | |
| 1100. | MGA | MGA_395 | 2364 | MAHLKTTAAHTQSFKQPSCTHISAD | −2.60 | 1.30E−02 |
| | | | | EKAAERSRKAPPIPLK | | |
| 1101. | MGA | MGA_396 | 2370 | MAAHTQSFKQPSCTHISADEKAAER | −1.87 | 7.98E−02 |
| | | | | SRKAPPIPLKLKPDYW | | |
| 1102. | MGA | MGA_398 | 2382 | MTHISADEKAAERSRKAPPIPLKLK | −2.43 | 3.28E−02 |
| | | | | PDYWSDKLQKEAEAFA | | |
| 1103. | MGA | MGA_399 | 2388 | MEKAAERSRKAPPIPLKLKPDYWSD | −1.95 | 7.10E−02 |
| | | | | KLQKEAEAFAYYRRTH | | |
| 1104. | MLH1 | MLH1_27 | 156 | MYNIATRRKALKNPSEEYGKILEVG | −2.36 | 2.32E−02 |
| | | | | RYSVHNAGISFSVKK | | |
| 1105. | MLH1 | MLH1_28 | 162 | MRKALKNPSEEYGKILEVVGRYSVH | −1.98 | 5.43E−02 |
| | | | | NAGISFSVKKQGETVA | | |
| 1106. | MLLT3 | MLLT3_44 | 258 | MMSKEPKPDSNLLTITSGQDKKAPS | −1.84 | 8.33E−02 |
| | | | | KRPPISDSEELSAKKR | | |
| 1107. | MLLT3 | MLLT3_45 | 264 | MPDSNLLTITSGQDKKAPSKRPPIS | −2.12 | 5.52E−02 |
| | | | | DSEELSAKKRKKSSSE | | |
| 1108. | MLLT3 | MLLT3_46 | 270 | MTITSGQDKKAPSKRPPISDSEELS | −1.97 | 6.92E−02 |
| | | | | AKKRKKSSSEALFKSF | | |
| 1109. | MLLT3 | MLLT3_47 | 276 | MDKKAPSKRPPISDSEELSAKKRKK | −1.87 | 7.74E−02 |
| | | | | SSSEALFKSFSSAPPL | | |
| 1110. | MLLT3 | MLLT3_73 | 432 | MRPVNRGGSRSRRVSLSDGSDSESS | −1.81 | 8.58E−02 |
| | | | | SASSPLHHEPPPPLLK | | |
| 1111. | MLLT3 | MLLT3_79 | 468 | MPLLKTNNNQILEVKSPIKQSKSDK | −1.79 | 4.14E−02 |
| | | | | QIKNGECDKAYLDELV | | |
| 1112. | MOS | MOS_23 | 132 | MSLGTIIMEFGGNVTLHQVIYGAAG | −2.51 | 2.39E−02 |
| | | | | HPEGDAGEPHCRTGGQ | | |
| 1113. | MOS | MOS_25 | 144 | MVTLHQVIYGAAGHPEGDAGEPHCR | −1.93 | 5.82E−02 |
| | | | | TGGQLSLGKCLKYSLD | | |
| 1114. | MSH2 | MSH2_11 | 60 | MQGVIKYMGPAGAKNLQSVVLSKMN | −2.24 | 4.52E−02 |
| | | | | FESFVKDLLLVRQYRV | | |
| 1115. | MSH2 | MSH2_12 | 66 | MMGPAGAKNLQSVVLSKMNFESFVK | −1.93 | 6.00E−02 |
| | | | | DLLLVRQYRVEVYKNR | | |
| 1116. | MSH2 | MSH2_107 | 636 | MSRHACVEVQDEIAFIPNDVYFEKD | −1.77 | 9.32E−02 |
| | | | | KQMFHIITGPNMGGKS | | |
| 1117. | MSH2 | MSH2_108 | 642 | MEVQDEIAFIPNDVYFEKDKQMFHI | −2.02 | 6.44E−02 |
| | | | | ITGPNMGGKSTYIRQT | | |
| 1118. | MSH6 | MSH6_25 | 144 | MKRLLKPYTGSKSKEAQKGGHFYSA | −3.02 | 6.71E−03 |
| | | | | KPEILRAMQRADEALN | | |
| 1119. | MSH6 | MSH6_26 | 150 | MYTGSKSKEAQKGGHFYSAKPEILR | −2.91 | 8.05E−03 |
| | | | | AMQRADEALNKDKIKR | | |
| 1120. | MSH6 | MSH6_30 | 174 | MAMQRADEALNKDKIKRLELAVCDE | −2.22 | 2.27E−02 |
| | | | | PSEPEEEEEMEVGTTY | | |
| 1121. | MSH6 | MSH6_124 | 738 | MTLNNLEIFLNGTNGSTEGTLLERV | −1.76 | 8.11E−02 |
| | | | | DTCHTPFGKRLLKQWL | | |
| 1122. | MSH6 | MSH6_125 | 744 | MIFLNGTNGSTEGTLLERVDTCHTP | −1.85 | 6.54E−02 |
| | | | | FGKRLLKQWLCAPLCN | | |
| 1123. | MSH6 | MSH6_136 | 810 | MLLKKLPDLERLLSKIHNVGSPLKS | −1.83 | 3.27E−02 |
| | | | | QNHPDSRAIMYEETTY | | |
| 1124. | MSH6 | MSH6_137 | 816 | MDLERLLSKIHNVGSPLKSQNHPDS | −1.91 | 3.50E−02 |
| | | | | RAIMYEETTYSKKKII | | |
| 1125. | MTOR | MTOR_39 | 228 | MQKPQWYRHTFEEAEKGFDETLAKE | −1.82 | 3.97E−02 |
| | | | | KGMNRDDRIHGALLIL | | |
| 1126. | MTOR | MTOR_42 | 246 | METLAKEKGMNRDDRIHGALLILNE | −3.37 | 3.68E−03 |
| | | | | LVRISSMEGERLREEM | | |
| 1127. | MTOR | MTOR_43 | 252 | MKGMNRDDRIHGALLILNELVRISS | −3.01 | 5.39E−03 |
| | | | | MEGERLREEMEEITQQ | | |
| 1128. | MTOR | MTOR_44 | 258 | MDRIHGALLILNELVRISSMEGERL | −3.27 | 4.35E−03 |
| | | | | REEMEEITQQQLVHDK | | |
| 1129. | MTOR | MTOR_47 | 276 | MMEGERLREEMEEITQQQLVHDKYC | −2.47 | 3.15E−02 |
| | | | | KDLMGFGTKPRHITPF | | |
| 1130. | MTOR | MTOR_137 | 816 | MVDELFIIIMDMLQDSSLLAKRQVA | −1.86 | 8.19E−02 |
| | | | | LWTLGQLVASTGYVVE | | |
| 1131. | MTOR | MTOR_138 | 822 | MIIMDMLQDSSLLAKRQVALWTLGQ | −3.00 | 6.94E−03 |
| | | | | LVASTGYVVEPYRKYP | | |
| 1132. | MTOR | MTOR_260 | 1554 | MDLFSLAQQCIDKARDLLDAELTAM | −2.82 | 9.27E−03 |
| | | | | AGESYSRAYGAMVSCH | | |
| 1133. | MTOR | MTOR_261 | 1560 | MQQCIDKARDLLDAELTAMAGESYS | −1.76 | 2.05E−02 |
| | | | | RAYGAMVSCHMLSELE | | |
| 1134. | MTOR | MTOR_280 | 1674 | MLLLGVDPSRQLDHPLPTVHPQVTY | −3.05 | 6.30E−03 |
| | | | | AYMKNMWKSARKIDAF | | |
| 1135. | MTRNR2L1 | MTRNR2L1_53 | 312 | MSYPSDNSTILFXGPHRQXHXRPRC | −1.99 | 6.76E−02 |
| | | | | WIRTSQWCSRYXRFVC | | |
| 1136. | MTRNR2L1 | MTRNR2L1_54 | 318 | MSTILFXGPHRQXHXRPRCWIRTSQ | −2.50 | 2.96E−02 |
| | | | | WCSRYXRFVCSTIRVL | | |
| 1137. | MTRNR2L1 | MTRNR2L1_55 | 324 | MGPHRQXHXRPRCWIRTSQWCSRYX | −2.06 | 3.15E−02 |
| | | | | RFVCSTIRVLCDLSSD | | |
| 1138. | MTRNR2L1 | MTRNR2L1_57 | 336 | MWIRTSQWCSRYXRFVCSTIRVLCD | −2.50 | 2.97E−02 |
| | | | | LSSDRSNPGWFLSILH | | |
| 1139. | MTRNR2L1 | MTRNR2L1_59 | 348 | MRFVCSTIRVLCDLSSDRSNPGWFL | −2.87 | 1.52E−02 |
| | | | | SILHFFQYERIREMGP | | |
| 1140. | MTRNR2L1 | MTRNR2L1_60 | 354 | MIRVLCDLSSDRSNPGWFLSILHFF | −2.45 | 3.26E−02 |
| | | | | QYERIREMGPTSXSAP | | |
| 1141. | MTRNR2L1 | MTRNR2L1_61 | 360 | MLSSDRSNPGWFLSILHFFQYERIR | −2.90 | 1.44E−02 |
| | | | | EMGPTSXSAPAPXMIL | | |
| 1142. | MTRNR2L10 | MTRNR2L10_30 | 174 | MNKXDEKTLCMELXFINANKNLSLQ | −2.65 | 1.20E−02 |
| | | | | ALAYYPCIKIFGWGDL | | |
| 1143. | MTRNR2L10 | MTRNR2L10_31 | 180 | MTLCMELXFINANKNLSLQALAYYP | −2.42 | 1.68E−02 |
| | | | | CIKIFGWGDLRAXFNL | | |
| 1144. | MTRNR2L10 | MTRNR2L10_32 | 186 | MXFINANKNLSLQALAYYPCIKIFG | −2.39 | 1.78E−02 |
| | | | | WGDLRAXFNLRATXTK | | |
| 1145. | MTRNR2L12 | MTRNR2L12_6 | 30 | MVNRRNRYSTARERXKIITKHNTAR | −2.50 | 3.03E−02 |
| | | | | TNPCTFCIMNXLEITL | | |
| 1146. | MTRNR2L12 | MTRNR2L12_7 | 36 | MYSTARERXKIITKHNTARTNPCTF | −1.93 | 7.33E−02 |
| | | | | CIMNXLEITLQREPKL | | |
| 1147. | MTRNR2L12 | MTRNR2L12_8 | 42 | MRXKIITKHNTARTNPCTFCIMNXL | −2.24 | 4.63E−02 |
| | | | | EITLQREPKLRPPKPD | | |
| 1148. | MTRNR2L12 | MTRNR2L12_17 | 96 | MAKXWEDLXVEATNLSSLVIAGCPR | −3.73 | 1.44E−03 |
| | | | | XNLSSTLNLPTEPSKS | | |
| 1149. | MTRNR2L12 | MTRNR2L12_18 | 102 | MLXVEATNLSSLVIAGCPRXNLSST | −3.06 | 1.12E−02 |
| | | | | LNLPTEPSKSPCKFNC | | |
| 1150. | MTRNR2L13 | MTRNR2L13_34 | 198 | MPIMLIXTFSIAYVYIRLEXPTDSX | −2.03 | 2.96E−02 |
| | | | | QPNINKXYNKHPIIYT | | |
| 1151. | MTRNR2L13 | MTRNR2L13_35 | 204 | MTFSIAYVYIRLEXPTDSXQPNINK | −2.15 | 2.49E−02 |
| | | | | XYNKHPIIYTVNPTKV | | |
| 1152. | MTRNR2L13 | MTRNR2L13_36 | 210 | MVYIRLEXPTDSXQPNINKXYNKHP | −1.96 | 2.70E−02 |
| | | | | IIYTVNPTKVCSKERL | | |
| 1153. | MTRNR2L13 | MTRNR2L13_37 | 216 | MXPTDSXQPNINKXYNKHPIIYTVN | −2.23 | 2.25E−02 |
| | | | | PTKVCSKERLQKVRGT | | |
| 1154. | MTRNR2L13 | MTRNR2L13_38 | 222 | MQPNINKXYNKHPIIYTVNPTKVCS | −2.45 | 1.61E−02 |
| | | | | KERLQKVRGTXQILPH | | |
| 1155. | MTRNR2L13 | MTRNR2L13_39 | 228 | MXYNKHPIIYTVNPTKVCSKERLQK | −2.32 | 1.97E−02 |
| | | | | VRGTXQILPHLFTKNI | | |
| 1156. | MTRNR2L3 | MTRNR2L3_44 | 258 | MLESISTIEFTTSMLDQDILMVXPL | −1.94 | 5.69E−02 |
| | | | | LRVCLFNDXSPMXSQF | | |
| 1157. | MTRNR2L3 | MTRNR2L3_46 | 270 | MMLDQDILMVXPLLRVCLFNDXSPM | −1.94 | 3.38E−02 |
| | | | | XSQFRPEXSRSVSTYL | | |
| 1158. | MTRNR2L3 | MTRNR2L3_48 | 282 | MLRVCLFNDXSPMXSQFRPEXSRSV | −3.01 | 1.09E−02 |
| | | | | STYLTFLLLRKDKRNR | | |
| 1159. | MTRNR2L3 | MTRNR2L3_49 | 288 | MNDXSPMXSQFRPEXSRSVSTYLTF | −2.32 | 3.94E−02 |
| | | | | LLLRKDKRNRAHFIKC | | |
| 1160. | MTRNR2L4 | MTRNR2L4_82 | 486 | MHPNSVAAIKGSFFQLLKSHVISVQ | −2.30 | 2.75E−02 |
| | | | | IGVIQVGFXLFNISPS | | |
| 1161. | MTRNR2L4 | MTRNR2L4_83 | 492 | MAIKGSFFQLLKSHVISVQIGVIQV | −2.35 | 2.54E−02 |
| | | | | GFXLFNISPSTKGQEK | | |
| 1162. | MTRNR2L4 | MTRNR2L4_84 | 498 | MFQLLKSHVISVQIGVIQVGFXLFN | −2.06 | 4.01E−02 |
| | | | | ISPSTKGQEKQGPFHK | | |
| 1163. | MTRNR2L4 | MTRNR2L4_85 | 504 | MHVISVQIGVIQVGFXLFNISPSTK | −2.40 | 2.19E−02 |
| | | | | GQEKQGPFHKAPLPP | | |
| 1164. | MTRNR2L5 | MTRNR2L5_10 | 54 | MSXKSTLIYVAKXXEDSLVAVTSLR | −3.04 | 1.16E−02 |
| | | | | RLVIADCPRXNFSSTL | | |
| 1165. | MTRNR2L5 | MTRNR2L5_11 | 60 | MIYVAKXXEDSLVAVTSLRRLVIAD | −3.44 | 4.92E−03 |
| | | | | CPRXNFSSTLNLTYRI | | |
| 1166. | MTRNR2L5 | MTRNR2L5_29 | 168 | MIIQXKQXCXCKXHXDIIHCISLHQ | −3.32 | 6.57E−03 |
| | | | | TRIIHXQLTTXYXQMI | | |
| 1167. | MTRNR2L5 | MTRNR2L5_30 | 174 | MXCXCKXHXDIIHCISLHQTRIIHX | −1.99 | 6.73E−02 |
| | | | | QLTTXYXQMINRHLII | | |
| 1168. | MTRNR2L5 | MTRNR2L5_31 | 180 | MHXDIIHCISLHQTRIIHXQLTTXY | −1.86 | 8.22E−02 |
| | | | | XQMINRHLIIFILTQY | | |
| 1169. | MTRNR2L6 | MTRNR2L6_4 | 18 | MFTQTKVXAKEILPRRSGRSTARXR | −1.93 | 3.39E−02 |
| | | | | XKSXPRIKXQGXTLIP | | |
| 1170. | MTRNR2L6 | MTRNR2L6_5 | 24 | MXAKEILPRRSGRSTARXRXKSXPR | −1.75 | 4.33E−02 |
| | | | | IKXQGXTLIPSAXXIN | | |
| 1171. | MTRNR2L6 | MTRNR2L6_6 | 30 | MPRRSGRSTARXRXKSXPRIKXQGX | −1.97 | 3.21E−02 |
| | | | | TLIPSAXXINXKXLYT | | |
| 1172. | MTSS1 | MTSS1_23 | 132 | MARQEIKKKSSDTLKLQKKAKKVDT | −3.03 | 6.51E−03 |
| | | | | LGRGDIQPQLDSALQD | | |
| 1173. | MTSS1 | MTSS1_24 | 138 | MKKSSDTLKLQKKAKKVDTLGRGDI | −2.97 | 7.26E−03 |
| | | | | QPQLDSALQDVNDKYL | | |
| 1174. | MTUS1 | MTUS1_48 | 282 | MEQKRRAREKANLKNPQIMYLEQEL | −2.35 | 1.88E−02 |
| | | | | ESLKAVLEIKNEKLHQ | | |
| 1175. | MTUS1 | MTUS1_49 | 288 | MREKANLKNPQIMYLEQELESLKAV | −2.41 | 1.71E−02 |
| | | | | LEIKNEKLHQQDIKLM | | |
| 1176. | MTUS1 | MTUS1_50 | 294 | MKNPQIMYLEQELESLKAVLEIKNE | −2.28 | 2.09E−02 |
| | | | | KLHQQDIKLMKMEKLV | | |
| 1177. | MYB | MYB_61 | 360 | MPGSLPEESASPARCMIVHQGTILD | −2.01 | 5.96E−02 |
| | | | | NVKNLLEFAETLQFID | | |
| 1178. | MYB | MYB_63 | 372 | MRCMIVHQGTILDNVKNLLEFAETL | −2.20 | 4.65E−02 |
| | | | | QFIDSFLNTSSNHENS | | |
| 1179. | MYC | MYC_64 | 378 | MDQIPELENNEKAPKVVILKKATAY | −1.77 | 9.05E−02 |
| | | | | ILSVQAEEQKLISEED | | |
| 1180. | MYC | MYC_65 | 384 | MENNEKAPKVVILKKATAYILSVQA | −2.50 | 1.50E−02 |
| | | | | EEQKLISEEDLLRKRR | | |
| 1181. | MYCL | MYCL_10 | 54 | MAPGIGPPEPWPGGCTGDEAESRGH | −3.14 | 7.45E−03 |
| | | | | SKGWGRNYASIIRRDC | | |
| 1182. | MYCL | MYCL_11 | 60 | MPEPWPGGCTGDEAESRGHSKGWGR | −2.68 | 1.36E−02 |
| | | | | NYASIIRRDCMWSGFS | | |
| 1183. | MYEOV | MYEOV15 | 84 | MALRVAVRGAFVSLWFAAGAGDRER | −5.22 | 4.89E−05 |
| | | | | NKGDKGAQTGAGLSQE | | |
| 1184. | MYEOV | MYEOV17 | 96 | MLWFAAGAGDRERNKGDKGAQTGAG | −1.89 | 3.91E−02 |
| | | | | LSQEAEDVDVSRARRV | | |
| 1185. | MYH11 | MYH11_53 | 312 | MGFVPIPAAQDDEMFQETVEAMAIM | −2.87 | 1.58E−02 |
| | | | | GFSEEEQLSILKVVSS | | |
| 1186. | MYH11 | MYH11_54 | 318 | MAAQDDEMFQETVEAMAIMGFSEEE | −3.10 | 5.83E−03 |
| | | | | QLSILKVVSSVLQLGN | | |
| 1187. | MYH11 | MYH11_139 | 828 | MLRNWQWWRLFTKVKPLLQVTRQEE | −2.67 | 1.17E−02 |
| | | | | EMQAKEDELQKTKERQ | | |
| 1188. | MYH11 | MYH11_140 | 834 | MWRLFTKVKPLLQVTRQEEEMQAKE | −2.54 | 1.42E−02 |
| | | | | DELQKTKERQQKAENE | | |
| 1189. | MYH11 | MYH11_148 | 882 | MSQLTEEKNLLQEQLQAETELYAEA | −2.49 | 1.53E−02 |
| | | | | EEMRVRLAAKKQELEE | | |
| 1190. | MYH11 | MYH11_149 | 888 | MKNLLQEQLQAETELYAEAEEMRVR | −2.24 | 2.20E−02 |
| | | | | LAAKKQELEEILHEME | | |
| 1191. | MYH11 | MYH11_150 | 894 | MQLQAETELYAEAEEMRVRLAAKKQ | −2.66 | 4.97E−03 |
| | | | | ELEEILHEMEARLEEE | | |
| 1192. | MYH11 | MYH11_174 | 1038 | MLEVRLKKEEKSRQELEKLKRKLEG | −1.94 | 3.36E−02 |
| | | | | DASDFHEQIADLQAQI | | |
| 1193. | MYH11 | MYH11_176 | 1050 | MQELEKLKRKLEGDASDFHEQIADL | −3.99 | 1.09E−03 |
| | | | | QAQIAELKMQLAKKEE | | |
| 1194. | MYH11 | MYH11_189 | 1128 | MSERAARNKAEKQKRDLGEELEALK | −3.36 | 3.73E−03 |
| | | | | TELEDTLDSTATQQEL | | |
| 1195. | MYH11 | MYH11_191 | 1140 | MKRDLGEELEALKTELEDTLDSTAT | −3.41 | 3.37E−03 |
| | | | | QQELRAKREQEVTVLK | | |
| 1196. | MYH11 | MYH11_229 | 1368 | MLNIQLSDSKKKLQDFASTVEALEE | −3.83 | 1.54E−03 |
| | | | | GKKRFQKEIENLTQQY | | |
| 1197. | MYH11 | MYH11_230 | 1374 | MDSKKKLQDFASTVEALEEGKKRFQ | −2.49 | 2.87E−02 |
| | | | | KEIENLTQQYEEKAAA | | |
| 1198. | MYH11 | MYH11_232 | 1386 | MVEALEEGKKRFQKEIENLTQQYEE | −3.31 | 4.79E−03 |
| | | | | KAAAYDKLEKTKNRLQ | | |
| 1199. | MYH9 | MYH9_25 | 144 | MEMPPHIYAITDTAYRSMMQDREDQ | −2.02 | 2.78E−02 |
| | | | | SILCTGESGAGKTENT | | |
| 1200. | MYH9 | MYH9_27 | 156 | MAYRSMMQDREDQSILCTGESGAGK | −4.35 | 5.03E−04 |
| | | | | TENTKKVIQYLAYVAS | | |
| 1201. | MYH9 | MYH9_50 | 294 | MEPYNKYRFLSNGHVTIPGQQDKDM | −3.55 | 2.02E−03 |
| | | | | FQETMEAMRIMGIPEE | | |
| 1202. | MYH9 | MYH9_51 | 300 | MRFLSNGHVTIPGQQDKDMFQETME | −4.14 | 8.04E−04 |
| | | | | AMRIMGIPEEEQMGLL | | |
| 1203. | MYH9 | MYH9_155 | 924 | MEEEEERCQHLQAEKKKMQQNIQEL | −3.69 | 2.02E−03 |
| | | | | EEQLEEEESARQKLQL | | |
| 1204. | MYH9 | MYH9_156 | 930 | MCQHLQAEKKKMQQNIQELEEQLEE | −2.56 | 1.09E−02 |
| | | | | EESARQKLQLEKVTTE | | |
| 1205. | MYH9 | MYH9_226 | 1350 | MAKHNLEKQIATLHAQVADMKKKME | −3.62 | 1.40E−03 |
| | | | | DSVGCLETAEEVKRKL | | |
| 1206. | MYH9 | MYH9_227 | 1356 | MKQIATLHAQVADMKKKMEDSVGCL | −2.45 | 6.91E−03 |
| | | | | ETAEEVKRKLQKDLEG | | |
| 1207. | MYH9 | MYH9_228 | 1362 | MHAQVADMKKKMEDSVGCLETAEEV | −3.00 | 3.35E−03 |
| | | | | KRKLQKDLEGLSQRHE | | |
| 1208. | MYH9 | MYH9_229 | 1368 | MMKKKMEDSVGCLETAEEVKRKLQK | −3.44 | 1.72E−03 |
| | | | | DLEGLSQRHEEKVAAY | | |
| 1209. | MYH9 | MYH9_233 | 1392 | MDLEGLSQRHEEKVAAYDKLEKTKT | −2.30 | 2.78E−02 |
| | | | | RLQQELDDLLVDLDHQ | | |
| 1210. | NCOA1 | NCOA1_40 | 234 | MDGEDFQSCLICIARRLPRPPAITG | −2.91 | 7.95E−03 |
| | | | | VESFMTKQDTTGKIIS | | |
| 1211. | NCOA1 | NCOA141 | 240 | MSCLICIARRLPRPPAITGVESFMT | −2.97 | 5.78E−03 |
| | | | | KQDTTGKIISIDTSSL | | |
| 1212. | NCOA1 | NCOA142 | 246 | MARRLPRPPAITGVESFMTKQDTTG | −2.98 | 7.08E−03 |
| | | | | KIISIDTSSLRAAGRT | | |
| 1213. | NCOA1 | NCOA196 | 570 | MSRLNIQPAKAESKDNKEIASILNE | −2.74 | 1.71E−02 |
| | | | | MIQSDNSSSDGKPLDS | | |
| 1214. | NCOA1 | NCOA198 | 582 | MKDNKEIASILNEMIQSDNSSSDGK | −3.37 | 5.90E−03 |
| | | | | PLDSGLLHNNDRLSDG | | |
| 1215. | NCOA1 | NCOA1_99 | 588 | MASILNEMIQSDNSSSDGKPLDSGL | −2.42 | 2.98E−02 |
| | | | | LHNNDRLSDGDSKYSQ | | |
| 1216. | NCOA4 | NCOA469 | 408 | MPCTSFAECVCDENCEKEALYKWLL | −1.86 | 4.71E−02 |
| | | | | KKEGKDKNGMPVEPKP | | |
| 1217. | NCOA4 | NCOA472 | 426 | MLYKWLLKKEGKDKNGMPVEPKPEP | −2.13 | 2.56E−02 |
| | | | | EKHKDSLNMWLCPRKE | | |
| 1218. | NCOA4 | NCOA473 | 432 | MKKEGKDKNGMPVEPKPEPEKHKDS | −2.19 | 2.37E−02 |
| | | | | LNMWLCPRKEVIEQTK | | |
| 1219. | NCOR1 | NCOR140 | 234 | MIVQIIYDENRKKAEEAHKIFEGLG | −4.57 | 2.95E−04 |
| | | | | PKVELPLYNQPSDTKV | | |
| 1220. | NCOR1 | NCOR141 | 240 | MDENRKKAEEAHKIFEGLGPKVELP | −3.07 | 7.62E−03 |
| | | | | LYNQPSDTKVYHENIK | | |
| 1221. | NCOR1 | NCOR142 | 246 | MAEEAHKIFEGLGPKVELPLYNQPS | −4.54 | 3.21E−04 |
| | | | | DTKVYHENIKTNQVMR | | |
| 1222. | NCOR1 | NCOR1114 | 678 | MLQQHKQKTSRKPREERDVSQCESV | −2.79 | 1.78E−02 |
| | | | | ASTVSAQEDEDIEASN | | |
| 1223. | NCOR1 | NCOR1_115 | 684 | MKTSRKPREERDVSQCESVASTVSA | −3.06 | 1.12E−02 |
| | | | | QEDEDIEASNEEENPE | | |
| 1224. | NCOR1 | NCOR1241 | 1440 | MENIKVVERGKYEDVKAGETVRSRH | −1.89 | 3.13E−02 |
| | | | | TSVVSSGPSVLRSTLH | | |
| 1225. | NCOR1 | NCOR1242 | 1446 | MERGKYEDVKAGETVRSRHTSVVSS | −2.06 | 2.64E−02 |
| | | | | GPSVLRSTLHEAPKAQ | | |
| 1226. | NCOR1 | NCOR1243 | 1452 | MDVKAGETVRSRHTSVVSSGPSVLR | −2.55 | 3.96E−03 |
| | | | | STLHEAPKAQLSPGIY | | |
| 1227. | NCOR1 | NCOR1285 | 1704 | MPPRPYNSASMSPGHPTHLAAAASA | −4.42 | 4.26E−04 |
| | | | | EREREREREKERERER | | |
| 1228. | NCOR1 | NCOR1287 | 1716 | MGHPTHLAAAASAEREREREREKER | −3.63 | 2.26E−03 |
| | | | | ERERIAAASSDLYLRP | | |
| 1229. | NCOR1 | NCOR1289 | 1728 | MEREREREREKERERERIAAASSDL | −1.83 | 5.13E−02 |
| | | | | YLRPGSEQPGRPGSHG | | |
| 1230. | NCOR1 | NCOR1_363 | 2172 | MDDKVEDHGVVMSQPMGVVPGTANT | −3.57 | 2.55E−03 |
| | | | | SVVTSGETRREEGDPS | | |
| 1231. | NCOR1 | NCOR1366 | 2190 | MPGTANTSVVTSGETRREEGDPSPH | −2.75 | 1.02E−02 |
| | | | | SGGVCKPKLISKSNSR | | |
| 1232. | NCOR1 | NCOR1368 | 2202 | METRREEGDPSPHSGGVCKPKLISK | −3.16 | 5.23E−03 |
| | | | | SNSRKSKSPIPGQGYL | | |
| 1233. | NET1 | NET1_14 | 78 | MLSSLDLKEPSNKRVRPLARVTSLA | −1.92 | 6.00E−02 |
| | | | | NLISPVRNGAVRRFGQ | | |
| 1234. | NET1 | NET1_15 | 84 | MKEPSNKRVRPLARVTSLANLISPV | −1.85 | 6.48E−02 |
| | | | | RNGAVRRFGQTIQSFT | | |
| 1235. | NF1 | NF1_29 | 168 | MEDNVDVHDIELLQYINVDCAKLKR | −1.83 | 8.61E−02 |
| | | | | LLKETAFKFKALKKVA | | |
| 1236. | NF1 | NF1_30 | 174 | MHDIELLQYINVDCAKLKRLLKETA | −1.93 | 7.37E−02 |
| | | | | FKFKALKKVAQLAVIN | | |
| 1237. | NF1 | NF1_32 | 186 | MCAKLKRLLKETAFKFKALKKVAQL | −1.86 | 8.23E−02 |
| | | | | AVINSLEKAFWNWVEN | | |
| 1238. | NF1 | NF1_43 | 252 | MVDGFAESTKRKAAVWPLQIILLIL | −2.19 | 4.12E−02 |
| | | | | CPEIIQDISKDVVDEN | | |
| 1239. | NF1 | NF1_44 | 258 | MSTKRKAAVWPLQIILLILCPEIIQ | −3.44 | 3.77E−03 |
| | | | | DISKDVVDENNMNKKL | | |
| 1240. | NF1 | NF1_45 | 264 | MAVWPLQIILLILCPEIIQDISKDV | −4.55 | 3.07E−04 |
| | | | | VDENNMNKKLFLDSLR | | |
| 1241. | NF1 | NF1_48 | 282 | MDISKDVVDENNMNKKLFLDSLRKA | −2.15 | 4.82E−02 |
| | | | | LAGHGGSRQLTESAAI | | |
| 1242. | NF1 | NF1_165 | 984 | MQASIETMMLNLVRYVRVLGNMVHA | −1.82 | 7.69E−02 |
| | | | | IQIKTKLCQLVEVMMA | | |
| 1243. | NF1 | NF1_167 | 996 | MRYVRVLGNMVHAIQIKTKLCQLVE | −3.40 | 3.43E−03 |
| | | | | VMMARRDDLSFCQEMK | | |
| 1244. | NF1 | NF1_255 | 1524 | MLGPPEHKPVADTHWSSLNLTSSKF | −3.37 | 3.62E−03 |
| | | | | EEFMTRHQVHEKEEFK | | |
| 1245. | NF1 | NF1_256 | 1530 | MKPVADTHWSSLNLTSSKFEEFMTR | −2.75 | 1.02E−02 |
| | | | | HQVHEKEEFKALKTLS | | |
| 1246. | NF1 | NF1_257 | 1536 | MHWSSLNLTSSKFEEFMTRHQVHEK | −2.90 | 8.06E−03 |
| | | | | EEFKALKTLSIFYQAG | | |
| 1247. | NF1 | NF1_258 | 1542 | MLTSSKFEEFMTRHQVHEKEEFKAL | −1.82 | 8.66E−02 |
| | | | | KTLSIFYQAGTSKAGN | | |
| 1248. | NF1 | NF1_264 | 1578 | MKAGNPIFYYVARRFKTGQINGDLL | −2.72 | 1.64E−02 |
| | | | | IYHVLLTLKPYYAKPY | | |
| 1249. | NF1 | NF1_265 | 1584 | MFYYVARRFKTGQINGDLLIYHVLL | −2.01 | 4.35E−02 |
| | | | | TLKPYYAKPYEIVVDL | | |
| 1250. | NF1 | NF1_271 | 1620 | MVVDLTHTGPSNRFKTDFLSKWFVV | −2.00 | 4.33E−02 |
| | | | | FPGFAYDNVSAVYIYN | | |
| 1251. | NF1 | NF1_272 | 1626 | MTGPSNRFKTDFLSKWFVVFPGFAY | −4.14 | 8.01E−04 |
| | | | | DNVSAVYIYNCNSWVR | | |
| 1252. | NF1 | NF1_273 | 1632 | MFKTDFLSKWFVVFPGFAYDNVSAV | −2.98 | 1.04E−02 |
| | | | | YIYNCNSWVREYTKYH | | |
| 1253. | NF2 | NF2_55 | 324 | MEMQRIKATAIRTEEEKRLMEQKVL | −2.66 | 1.19E−02 |
| | | | | EAEVLALKMAEESERR | | |
| 1254. | NF2 | NF2_56 | 330 | MATAIRTEEEKRLMEQKVLEAEVLA | −1.75 | 7.70E−02 |
| | | | | LKMAEESERRAKEADQ | | |
| 1255. | NFE2L2 | NFE2L22 | 6 | MPPPGLPSQQDMDLIDILWRQDIDL | −1.93 | 7.11E−02 |
| | | | | GVSREVFDFSQRRKEY | | |
| 1256. | NFE2L2 | NFE2L23 | 12 | MSQQDMDLIDILWRQDIDLGVSREV | −1.82 | 8.33E−02 |
| | | | | FDFSQRRKEYELEKQK | | |
| 1257. | NFE2L2 | NFE2L24 | 18 | MLIDILWRQDIDLGVSREVFDFSQR | −2.28 | 4.26E−02 |
| | | | | RKEYELEKQKKLEKER | | |
| 1258. | NFE2L2 | NFE2L25 | 24 | MRQDIDLGVSREVFDFSQRRKEYEL | −2.01 | 6.33E−02 |
| | | | | EKQKKLEKERQEQLQK | | |
| 1259. | NFKB2 | NFKB2_19 | 108 | MLVGKQCSELGICAVSVGPKDMTAQ | −3.56 | 3.86E−03 |
| | | | | FNNLGVLHVTKKNMMG | | |
| 1260. | NFKB2 | NFKB2_20 | 114 | MSELGICAVSVGPKDMTAQFNNLGV | −2.96 | 7.40E−03 |
| | | | | LHVTKKNMMGTMIQKL | | |
| 1261. | NFKB2 | NFKB2_21 | 120 | MAVSVGPKDMTAQFNNLGVLHVTKK | −3.00 | 6.84E−03 |
| | | | | NMMGTMIQKLQRQRLR | | |
| 1262. | NFKB2 | NFKB2_90 | 534 | MVITGQTSVVSFLLRVGADPALLDR | −1.79 | 4.14E−02 |
| | | | | HGDSAMHLALRAGAGA | | |
| 1263. | NFKB2 | NFKB2_91 | 540 | MSVVSFLLRVGADPALLDRHGDSAM | −3.45 | 2.95E−03 |
| | | | | HLALRAGAGAPELLRA | | |
| 1264. | NIPBL | NIPBL_145 | 864 | MKLERKHRHESGDSRERPSSGEQKS | −3.69 | 2.01E−03 |
| | | | | RPDSPRVKQGDSNKSR | | |
| 1265. | NIPBL | NIPBL_146 | 870 | MRHESGDSRERPSSGEQKSRPDSPR | −3.70 | 1.97E−03 |
| | | | | VKQGDSNKSRSDKLGF | | |
| 1266. | NIPBL | NIPBL_147 | 876 | MSRERPSSGEQKSRPDSPRVKQGDS | −2.88 | 1.55E−02 |
| | | | | NKSRSDKLGFKSPTSK | | |
| 1267. | NIPBL | NIPBL_159 | 948 | MRSGALKNFVIPKIKRDKDGNVTQE | −3.04 | 6.48E−03 |
| | | | | TKKMEMKGEPKDKVEK | | |
| 1268. | NIPBL | NIPBL_160 | 954 | MNFVIPKIKRDKDGNVTQETKKMEM | −3.07 | 6.09E−03 |
| | | | | KGEPKDKVEKIGLVED | | |
| 1269. | NIPBL | NIPBL_183 | 1092 | MDNDSDEAFESSRKRHKKDDDKAWE | −2.67 | 1.93E−02 |
| | | | | YEERDRRSSGDHRRSG | | |
| 1270. | NIPBL | NIPBL_184 | 1098 | MAFESSRKRHKKDDDKAWEYEERDR | −2.55 | 2.48E−02 |
| | | | | RSSGDHRRSGHSHEGR | | |
| 1271. | NIPBL | NIPBL_185 | 1104 | MKRHKKDDDKAWEYEERDRRSSGDH | −2.29 | 4.00E−02 |
| | | | | RRSGHSHEGRRSSGGG | | |
| 1272. | NIPBL | NIPBL_186 | 1110 | MDDKAWEYEERDRRSSGDHRRSGHS | −2.20 | 4.63E−02 |
| | | | | HEGRRSSGGGRYRNRS | | |
| 1273. | NIPBL | NIPBL_202 | 1206 | MENILDNLEDMDFTAFGDDDEIPQE | −2.28 | 3.44E02 |
| | | | | LLLGKHQLNELGSESA | | |
| 1274. | NIPBL | NIPBL_203 | 1212 | MLEDMDFTAFGDDDEIPQELLLGKH | −2.05 | 4.84E−02 |
| | | | | QLNELGSESAKIKAMG | | |
| 1275. | NIPBL | NIPBL_308 | 1842 | MPQLAEQYYDMLIERILDTGISVRK | −2.73 | 8.62E−03 |
| | | | | RVIKILRDICIEQPTF | | |
| 1276. | NIPBL | NIPBL_309 | 1848 | MYYDMLIERILDTGISVRKRVIKIL | −2.86 | 8.64E−03 |
| | | | | RDICIEQPTFPKITEM | | |
| 1277. | NIPBL | NIPBL_424 | 2538 | MVSQGILLLLMLKQHLKNLCGFSDS | −1.85 | 8.18E−02 |
| | | | | KIQKYSPSESAKVYDK | | |
| 1278. | NIPBL | NIPBL_426 | 2550 | MQHLKNLCGFSDSKIQKYSPSESAK | −1.91 | 7.31E−02 |
| | | | | VYDKAINRKTGVHFHP | | |
| 1279. | NIPBL | NIPBL_436 | 2610 | MKRSIVKQYLDFKLLMEHLDPDEEE | −1.97 | 6.47E−02 |
| | | | | EEGEVSASTNARNKAI | | |
| 1280. | NOTCH1 | NOTCH1_43 | 252 | MNCEENIDDCPGNNCKNGGACVDGV | −2.22 | 2.26E−02 |
| | | | | NTYNCRCPPEWTGQYC | | |
| 1281. | NOTCH1 | NOTCH145 | 264 | MNCKNGGACVDGVNTYNCRCPPEWT | −4.47 | 2.02E−04 |
| | | | | GQYCTEDVDECQLMPN | | |
| 1282. | NOTCH1 | NOTCH146 | 270 | MACVDGVNTYNCRCPPEWTGQYCTE | −5.04 | 8.39E−05 |
| | | | | DVDECQLMPNACQNGG | | |
| 1283. | NOTCH1 | NOTCH147 | 276 | MNTYNCRCPPEWTGQYCTEDVDECQ | −1.98 | 4.60E−02 |
| | | | | LMPNACQNGGTCHNTH | | |
| 1284. | NOTCH1 | NOTCH177 | 456 | MVSNPCQNDATCLDQIGEFQCICMP | −3.55 | 2.64E−03 |
| | | | | GYEGVHCEVNTDECAS | | |
| 1285. | NOTCH1 | NOTCH179 | 468 | MDQIGEFQCICMPGYEGVHCEVNTD | −2.19 | 1.04E−02 |
| | | | | ECASSPCLHNGRCLDK | | |
| 1286. | NOTCH1 | NOTCH1151 | 900 | MGTFCEEDINECASDPCRNGANCTD | −1.88 | 2.13E−02 |
| | | | | CVDSYTCTCPAGFSGI | | |
| 1287. | NOTCH1 | NOTCH1_153 | 912 | MSDPCRNGANCTDCVDSYTCTCPAG | −5.27 | 4.21E−05 |
| | | | | FSGIHCENNTPDCTES | | |
| 1288. | NOTCH1 | NOTCH1351 | 2100 | MLCSPNGYLGSLKPGVQGKKVRKPS | −4.01 | 1.05E−03 |
| | | | | SKGLACGSKEAKDLKA | | |
| 1289. | NOTCH1 | NOTCH1352 | 2106 | MYLGSLKPGVQGKKVRKPSSKGLAC | −2.75 | 1.02E−02 |
| | | | | GSKEAKDLKARRKKSQ | | |
| 1290. | NOTCH1 | NOTCH1354 | 2118 | MKVRKPSSKGLACGSKEAKDLKARR | −2.51 | 1.48E−02 |
| | | | | KKSQDGKGCLLDSSGM | | |
| 1291. | NOTCH2 | NOTCH218 | 102 | MYSTSHPCFVSRPCLNGGTCHMLSR | −5.45 | 2.36E−05 |
| | | | | DTYECTCQVGFTGKEC | | |
| 1292. | NOTCH2 | NOTCH219 | 108 | MCFVSRPCLNGGTCHMLSRDTYECT | −5.19 | 5.35E−05 |
| | | | | CQVGFTGKECQWTDAC | | |
| 1293. | NOTCH2 | NOTCH221 | 120 | MCHMLSRDTYECTCQVGFTGKECQW | −2.82 | 1.73E−02 |
| | | | | TDACLSHPCANGSTCT | | |
| 1294. | NOTCH2 | NOTCH222 | 126 | MDTYECTCQVGFTGKECQWTDACLS | −3.17 | 5.18E−04 |
| | | | | HPCANGSTCTTVANQF | | |
| 1295. | NOTCH2 | NOTCH2192 | 1146 | MEEQLDECASNPCQHGATCSDFIGG | −5.98 | 3.52E−06 |
| | | | | YRCECVPGYQGVNCEY | | |
| 1296. | NOTCH2 | NOTCH2193 | 1152 | MCASNPCQHGATCSDFIGGYRCECV | −4.20 | 1.42E−05 |
| | | | | PGYQGVNCEYEVDECQ | | |
| 1297. | NOTCH2 | NOTCH2194 | 1158 | MQHGATCSDFIGGYRCECVPGYQGV | −4.70 | 2.14E−04 |
| | | | | NCEYEVDECQNQPCQN | | |
| 1298. | NOTCH2 | NOTCH2198 | 1182 | MNCEYEVDECQNQPCQNGGTCIDLV | −2.76 | 1.94E−02 |
| | | | | NHFKCSCPPGTRGMKS | | |
| 1299. | NRAS | NRAS_7 | 36 | MEDSYRKQVVIDGETCLLDILDTAG | −3.00 | 1.23E−02 |
| | | | | QEEYSAMRDQYMRTGE | | |
| 1300. | NRAS | NRAS_9 | 48 | METCLLDILDTAGQEEYSAMRDQYM | −2.70 | 1.82E−02 |
| | | | | RTGEGFLCVFAINNSK | | |
| 1301. | NSD1 | NSD1_9 | 48 | MPLKYEVGDLIWAKFKRRPWWPCRI | −2.12 | 5.55E−02 |
| | | | | CSDPLINTHSKMKVSN | | |
| 1302. | NSD1 | NSD1_10 | 54 | MGDLIWAKFKRRPWWPCRICSDPLI | −1.84 | 8.06E−02 |
| | | | | NTHSKMKVSNRRPYRQ | | |
| 1303. | NSD1 | NSD1_11 | 60 | MKFKRRPWWPCRICSDPLINTHSKM | −1.90 | 7.64E−02 |
| | | | | KVSNRRPYRQYYVEAF | | |
| 1304. | NSD1 | NSD1_24 | 138 | MGYRHKVPQKILSKWEASVGLAEQY | −2.53 | 2.67E−02 |
| | | | | DVPKGSKNRKCIPGSI | | |
| 1305. | NSD1 | NSD1_25 | 144 | MPQKILSKWEASVGLAEQYDVPKGS | −3.03 | 1.18E−02 |
| | | | | KNRKCIPGSIKLDSEE | | |
| 1306. | NSD1 | NSD1_38 | 222 | MKPCAKSRARKSSDNPKRTSVKKGH | −2.70 | 1.83E−02 |
| | | | | IQFEAHKDERRGKIPE | | |
| 1307. | NSD1 | NSD1_39 | 228 | MRARKSSDNPKRTSVKKGHIQFEAH | −2.46 | 2.70E−02 |
| | | | | KDERRGKIPENLGLNF | | |
| 1308. | NSD1 | NSD1_40 | 234 | MDNPKRTSVKKGHIQFEAHKDERRG | −1.98 | 6.34E−02 |
| | | | | KIPENLGLNFISGDIS | | |
| 1309. | NSD1 | NSD1_41 | 240 | MSVKKGHIQFEAHKDERRGKIPENL | −1.75 | 8.43E−02 |
| | | | | GLNFISGDISDTQASN | | |
| 1310. | NSD1 | NSD1_109 | 648 | MHDSKTKEQRLMTAQNLVSYRSPGR | −2.88 | 1.02E−02 |
| | | | | GDCSTNSPVGVSKVLV | | |
| 1311. | NSD1 | NSD1_129 | 768 | MMVKNTVNRKALKTERKRKLNQLPS | −3.54 | 2.66E−03 |
| | | | | VTLDAVLQGDRERGGS | | |
| 1312. | NSD1 | NSD1_130 | 774 | MNRKALKTERKRKLNQLPSVTLDAV | −1.95 | 6.35E−02 |
| | | | | LQGDRERGGSLRGGAE | | |
| 1313. | NSD1 | NSD1_320 | 1914 | MKQHREGMLFISKLDGRLSCTEHDP | −1.80 | 4.71E−02 |
| | | | | CGPNPLEPGEIREYVP | | |
| 1314. | NSD1 | NSD1_323 | 1932 | MCTEHDPCGPNPLEPGEIREYVPPP | −3.87 | 1.42E−03 |
| | | | | VPLPPGPSTHLAEQST | | |
| 1315. | NSD2 | NSD2_43 | 252 | MKGQKKSARQYHVQFFGDAPERAWI | −2.08 | 5.48E−02 |
| | | | | FEKSLVAFEGEGQFEK | | |
| 1316. | NSD2 | NSD2_44 | 258 | MARQYHVQFFGDAPERAWIFEKSLV | −2.18 | 4.79E−02 |
| | | | | AFEGEGQFEKLCQESA | | |
| 1317. | NSD2 | NSD2_126 | 750 | MVFESRGFRCPLHSCVSCHASNPSN | −4.05 | 2.83E−04 |
| | | | | PRPSKGKMMRCVRCPV | | |
| 1318. | NSD2 | NSD2_130 | 774 | MPRPSKGKMMRCVRCPVAYHSGDAC | −5.63 | 1.29E−05 |
| | | | | LAAGCSVIASNSIICT | | |
| 1319. | NSD2 | NSD2_160 | 954 | MGRVFKNALQEAEARFREIKLQREA | −2.25 | 4.08E−02 |
| | | | | RETQESERKPPPYKHI | | |
| 1320. | NSD2 | NSD2_161 | 960 | MALQEAEARFREIKLQREARETQES | −2.01 | 5.08E−02 |
| | | | | ERKPPPYKHIKVNKPY | | |
| 1321. | NSD2 | NSD2_185 | 1104 | MRIKHAHENDITHFYMLTIDKDRII | −1.78 | 8.82E−02 |
| | | | | DAGPKGNYSRFMNHSC | | |
| 1322. | NSD2 | NSD2_187 | 1116 | MFYMLTIDKDRIIDAGPKGNYSRFM | −3.07 | 6.16E−03 |
| | | | | NHSCQPNCETLKWTVN | | |
| 1323. | NSD3 | NSD3_100 | 594 | MPKKKIKKEQVETVPQATVKTGLQK | −3.31 | 3.16E−03 |
| | | | | GASEISDSCKPLKKRS | | |
| 1324. | NSD3 | NSD3_101 | 600 | MKEQVETVPQATVKTGLQKGASEIS | −2.53 | 2.85E−02 |
| | | | | DSCKPLKKRSRASTDV | | |
| 1325. | NSD3 | NSD3_104 | 618 | MGASEISDSCKPLKKRSRASTDVEM | −1.79 | 8.64E−02 |
| | | | | TSSAYRDTSDSDSRGL | | |
| 1326. | NSD3 | NSD3_171 | 1020 | MEGDKSFAEGQTSINKTFKKALEEA | −1.80 | 8.17E−02 |
| | | | | AKRFQELKAQRESKEA | | |
| 1327. | NSD3 | NSD3_172 | 1026 | MAEGQTSINKTFKKALEEAAKRFQE | −1.92 | 6.75E−02 |
| | | | | LKAQRESKEALEIEKN | | |
| 1328. | NSD3 | NSD3_173 | 1032 | MINKTFKKALEEAAKRFQELKAQRE | −1.82 | 7.88E−02 |
| | | | | SKEALEIEKNSRKPPP | | |
| 1329. | NSD3 | NSD3_192 | 1146 | MAEIIKTERRGWGLRTKRSIKKGEF | −2.56 | 2.68E−02 |
| | | | | VNEYVGELIDEEECRL | | |
| 1330. | NSD3 | NSD3_195 | 1164 | MIKKGEFVNEYVGELIDEEECRLRI | −2.30 | 4.20E−02 |
| | | | | KRAHENSVTNFYMLTV | | |
| 1331. | NTRK1 | NTRK1_112 | 666 | MGDFGMSRDIYSTDYYRVGGRTMLP | −2.13 | 5.62E−04 |
| | | | | IRWMPPESILYRKFTT | | |
| 1332. | NTRK1 | NTRK1_119 | 708 | MDVWSFGVVLWEIFTYGKQPWYQLS | −4.22 | 6.67E−04 |
| | | | | NTEAIDCITQGRELER | | |
| 1333. | NUP133 | NUP13372 | 426 | MVCSTGTGKFSLPQEKIVFNAQGDS | −2.03 | 6.34E−02 |
| | | | | VLGAGACGGVPIIFSR | | |
| 1334. | NUP133 | NUP13373 | 432 | MGKFSLPQEKIVFNAQGDSVLGAGA | −2.26 | 4.49E−02 |
| | | | | CGGVPIIFSRNSGLVS | | |
| 1335. | NUP133 | NUP133113 | 672 | MNKREYEIPSNLTPADVFFREVSQV | −3.20 | 4.87E−03 |
| | | | | DTICECLLEHEEQVLR | | |
| 1336. | NUP133 | NUP133116 | 690 | MREVSQVDTICECLLEHEEQVLRDA | −1.75 | 4.33E−02 |
| | | | | PMDSIEWAEVVINVNN | | |
| 1337. | NUP214 | NUP214112 | 666 | MPRITPPAAKPGSPQAKSLQPAVAE | −2.48 | 1.55E−02 |
| | | | | KQGHQWKDSDPVMAGI | | |
| 1338. | NUP214 | NUP214113 | 672 | MAAKPGSPQAKSLQPAVAEKQGHQW | −1.97 | 2.83E−02 |
| | | | | KDSDPVMAGIGEEIAH | | |
| 1339. | NUP214 | NUP214122 | 726 | MACFQVGTSEEMKMLRTESDDLHTF | −2.62 | 1.26E−02 |
| | | | | LLEIKETTESLHGDIS | | |
| 1340. | NUP214 | NUP214123 | 732 | MTSEEMKMLRTESDDLHTFLLEIKE | −1.82 | 8.63E−02 |
| | | | | TTESLHGDISSLKTTL | | |
| 1341. | NUP93 | NUP93_116 | 690 | MLLLDLITFFDEYHSGHIDRAFDII | −2.00 | 5.96E−02 |
| | | | | ERLKLVPLNQESVEER | | |
| 1342. | NUP93 | NUP93_117 | 696 | MTFFDEYHSGHIDRAFDIIERLKLV | −2.00 | 6.27E−02 |
| | | | | PLNQESVEERVAAFRN | | |
| 1343. | OLIG2 | OLIG2_44 | 258 | MGLLKSPSAAAAAPLGGGGGGSGAS | −4.19 | 7.14E−04 |
| | | | | GGFQHWGGMPCPCSMC | | |
| 1344. | OLIG2 | OLIG2_45 | 264 | MSAAAAAPLGGGGGGSGASGGFQHW | −2.35 | 2.73E−02 |
| | | | | GGMPCPCSMCQVPPPH | | |
| 1345. | PAK2 | PAK2_69 | 408 | MWMAPEVVTRKAYGPKVDIWSLGIM | −3.50 | 2.86E−03 |
| | | | | AIEMVEGEPPYLNENP | | |
| 1346. | PAK2 | PAK2_70 | 414 | MVTRKAYGPKVDIWSLGIMAIEMVE | −3.37 | 3.65E−03 |
| | | | | GEPPYLNENPLRALYL | | |
| 1347. | PALB2 | PALB2_89 | 528 | MPLLPTSSLSIVNRSKEEVTSHKYQ | −2.15 | 5.20E−02 |
| | | | | HEKLFIQVKGKKSRHQ | | |
| 1348. | PALB2 | PALB2_90 | 534 | MSLSIVNRSKEEVTSHKYQHEKLFI | −2.77 | 8.09E−03 |
| | | | | QVKGKKSRHQKEDSLS | | |
| 1349. | PARK7 | PARK7_8 | 42 | MPVQCSRDVVICPDASLEDAKKEGP | −3.22 | 4.73E−03 |
| | | | | YDVVVLPGGNLGAQNL | | |
| 1350. | PARK7 | PARK7_10 | 54 | MDASLEDAKKEGPYDVVVLPGGNLG | −2.96 | 7.34E−03 |
| | | | | AQNLSESAAVKEILKE | | |
| 1351. | PARK7 | PARK7_11 | 60 | MAKKEGPYDVVVLPGGNLGAQNLSE | −2.08 | 5.73E−02 |
| | | | | SAAVKEILKEQENRKG | | |
| 1352. | PATZ1 | PATZ1_2 | 6 | MASCGPSGCYTYQVSRHSTEMLHNL | −2.48 | 1.79E−04 |
| | | | | NQQRKNGGRFCDVLLR | | |
| 1353. | PATZ1 | PATZ1_3 | 12 | MGCYTYQVSRHSTEMLHNLNQQRKN | −2.73 | 1.82E−02 |
| | | | | GGRFCDVLLRVGDESF | | |
| 1354. | PATZ1 | PATZ1_53 | 312 | MQHGVTSLQLGYIDLPPPRLGENGL | −3.57 | 2.25E−03 |
| | | | | PISEDPDGPRKRSRTR | | |
| 1355. | PATZ1 | PATZ1_54 | 318 | MLQLGYIDLPPPRLGENGLPISEDP | −2.60 | 2.31E−02 |
| | | | | DGPRKRSRTRKQVACE | | |
| 1356. | PBRM1 | PBRM1_138 | 822 | MPAVDPNFPNKPPLTFDIIRKNVEN | −2.08 | 5.52E−02 |
| | | | | NRYRRLDLFQEHMFEV | | |
| 1357. | PBRM1 | PBRM1_140 | 834 | MLTFDIIRKNVENNRYRRLDLFQEH | −1.78 | 8.07E−02 |
| | | | | MFEVLERARRMNRTDS | | |
| 1358. | PBRM1 | PBRM1_171 | 1020 | MHLATRKFLEKEVFKSDYYNKVPVS | −3.11 | 6.93E−03 |
| | | | | KILGKCVVMFVKEYFK | | |
| 1359. | PBRM1 | PBRM1_176 | 1050 | MVVMFVKEYFKLCPENFRDEDVFVC | −2.40 | 2.17E−02 |
| | | | | ESRYSAKTKSFKKIKL | | |
| 1360. | PBRM1 | PBRM1_177 | 1056 | MEYFKLCPENFRDEDVFVCESRYSA | −2.21 | 4.37E−02 |
| | | | | KTKSFKKIKLWTMPIS | | |
| 1361. | PBX1 | PBX1_21 | 120 | MEKGGGSAAAAAAAAASGGAGSDNS | −3.76 | 2.61E−03 |
| | | | | VEHSDYRAKLSQIRQI | | |
| 1362. | PBX1 | PBX1_22 | 126 | MAAAAAAAAASGGAGSDNSVEHSDY | −3.51 | 4.28E−03 |
| | | | | RAKLSQIRQIYHTELE | | |
| 1363. | PBX1 | PBX1_26 | 150 | MRAKLSQIRQIYHTELEKYEQACNE | −2.30 | 2.01E−02 |
| | | | | FTTHVMNLLREQSRTR | | |
| 1364. | PCM1 | PCM1_32 | 186 | MCQVSEEDGRGEPAMESSQIVSRLV | −1.89 | 5.19E−02 |
| | | | | QIRDYITKASSMREDL | | |
| 1365. | PCM1 | PCM1_33 | 192 | MDGRGEPAMESSQIVSRLVQIRDYI | −2.39 | 2.84E−02 |
| | | | | TKASSMREDLVEKNER | | |
| 1366. | PCM1 | PCM1_54 | 318 | MHDLLKRMLQQQEQLRALQGRQAAL | −2.23 | 4.61E−02 |
| | | | | LALQHKAEQAIAVMDD | | |
| 1367. | PCM1 | PCM1_57 | 336 | MGRQAALLALQHKAEQAIAVMDDSV | −3.34 | 3.82E−03 |
| | | | | VAETAGSLSGVSITSE | | |
| 1368. | PDGFB | PDGFB_20 | 114 | MNANFLVWPPCVEVQRCSGCCNNRN | −2.97 | 1.16E−03 |
| | | | | VQCRPTQVQLRPVQVR | | |
| 1369. | PDGFB | PDGFB_25 | 144 | MQVQLRPVQVRKIEIVRKKPIFKKA | −1.82 | 6.17E−02 |
| | | | | TVTLEDHLACKCETVA | | |
| 1370. | PDGFB | PDGFB_27 | 156 | MEIVRKKPIFKKATVTLEDHLACKC | −2.69 | 2.17E−02 |
| | | | | ETVAAARPVTRSPGGS | | |
| 1371. | PDGFD | PDGFD48 | 282 | MNVVFFPRCLLVQRCGGNCGCGTVN | −1.85 | 4.41E−02 |
| | | | | WRSCTCNSGKTVKKYH | | |
| 1372. | PDGFD | PDGFD_51 | 300 | MGCGTVNWRSCTCNSGKTVKKYHEV | −3.86 | 1.45E−03 |
| | | | | LQFEPGHIKRRGRAKT | | |
| 1373. | PDGFD | PDGFD53 | 312 | MNSGKTVKKYHEVLQFEPGHIKRRG | −2.85 | 1.55E−02 |
| | | | | RAKTMALVDIQLDHHE | | |
| 1374. | PDGFRB | PDGFRB121 | 720 | MPVGLPLPSHVSLTGESDGGYMDMS | −2.14 | 4.61E−02 |
| | | | | KDESVDYVPMLDMKGD | | |
| 1375. | PDGFRB | PDGFRB122 | 726 | MPSHVSLTGESDGGYMDMSKDESVD | −2.97 | 7.52E−03 |
| | | | | YVPMLDMKGDVKYADI | | |
| 1376. | PDGFRB | PDGFRB131 | 780 | MSAPERTCRATLINESPVLSYMDLV | −2.08 | 3.43E−02 |
| | | | | GFSYQVANGMEFLASK | | |
| 1377. | PDGFRB | PDGFRB134 | 798 | MSYMDLVGFSYQVANGMEFLASKNC | −1.86 | 5.95E−02 |
| | | | | VHRDLAARNVLICEGK | | |
| 1378. | PDS5B | PDS5B_43 | 252 | MEHVFDLILELYNIDSHLLLSVLPQ | −2.24 | 1.81E−02 |
| | | | | LEFKLKSNDNEERLQV | | |
| 1379. | PDS5B | PDS5B_44 | 258 | MILELYNIDSHLLLSVLPQLEFKLK | −2.04 | 3.67E−02 |
| | | | | SNDNEERLQVVKLLAK | | |
| 1380. | PDS5B | PDS5B_46 | 270 | MLSVLPQLEFKLKSNDNEERLQVVK | −2.76 | 1.50E−02 |
| | | | | LLAKMFGAKDSELASQ | | |
| 1381. | PDS5B | PDS5B_47 | 276 | MLEFKLKSNDNEERLQVVKLLAKMF | −3.16 | 5.25E−03 |
| | | | | GAKDSELASQNKPLWQ | | |
| 1382. | PDS5B | PDS5B_79 | 468 | METTERMKCLYYLYATLDLNAVKYV | −2.42 | 3.32E−02 |
| | | | | SNIKFCSFHPLQYIGF | | |
| 1383. | PDS5B | PDS5B_80 | 474 | MKCLYYLYATLDLNAVKYVSNIKFC | −2.27 | 4.00E−02 |
| | | | | SFHPLQYIGFYGKETT | | |
| 1384. | PDS5B | PDS5B_81 | 480 | MYATLDLNAVKYVSNIKFCSFHPLQ | −3.53 | 2.72E−03 |
| | | | | YIGFYGKETTNTCILK | | |
| 1385. | PDS5B | PDS5B_82 | 486 | MNAVKYVSNIKFCSFHPLQYIGFYG | −3.64 | 2.23E−03 |
| | | | | KETTNTCILKCNLCSV | | |
| 1386. | PGR | PGR_102 | 606 | MNDCIVDKIRRKNCPACRLRKCCQA | −2.44 | 3.12E−02 |
| | | | | GMVLGGRKFKKFNKVR | | |
| 1387. | PGR | PGR_103 | 612 | MKIRRKNCPACRLRKCCQAGMVLGG | −2.20 | 4.40E−02 |
| | | | | RKFKKFNKVRVVRALD | | |
| 1388. | PGR | PGR_126 | 750 | MIQYSWMSLMVFGLGWRSYKHVSGQ | −2.03 | 4.65E−02 |
| | | | | MLYFAPDLILNEQRMK | | |
| 1389. | PGR | PGR_127 | 756 | MSLMVFGLGWRSYKHVSGQMLYFAP | −3.38 | 2.81E−03 |
| | | | | DLILNEQRMKESSFYS | | |
| 1390. | PHB | PHB_32 | 186 | MQVAQQEAERARFVVEKAEQQKKAA | −3.44 | 3.21E−03 |
| | | | | IISAEGDSKAAELIAN | | |
| 1391. | PHB | PHB_33 | 192 | MAERARFVVEKAEQQKKAAIISAEG | −4.23 | 6.61E−04 |
| | | | | DSKAAELIANSLATAG | | |
| 1392. | PHLPP1 | PHLPP160 | 354 | MLDICGYFLKALYASSNELVQLDVY | −1.98 | 4.96E−02 |
| | | | | PVPNYLSYMDVSRNRL | | |
| 1393. | PHLPP1 | PHLPP162 | 366 | MASSNELVQLDVYPVPNYLSYMDVS | −2.09 | 5.79E−02 |
| | | | | RNRLENVPEWVCESRK | | |
| 1394. | PHLPP1 | PHLPP163 | 372 | MVQLDVYPVPNYLSYMDVSRNRLEN | −1.78 | 8.87E−02 |
| | | | | VPEWVCESRKLEVLDI | | |
| 1395. | PICALM | PICALM_50 | 294 | MAASRATTLSNAVSSLASTGLSLTK | −3.82 | 1.55E−03 |
| | | | | VDEREKQAALEEEQAR | | |
| 1396. | PICALM | PICALM54 | 318 | MVDEREKQAALEEEQARLKALKEQR | −2.05 | 6.12E−02 |
| | | | | LKELAKKPHTSLTTAA | | |
| 1397. | PICALM | PICALM58 | 342 | MLKELAKKPHTSLTTAASPVSTSAG | −1.97 | 2.72E−02 |
| | | | | GIMTAPAIDIFSTPSS | | |
| 1398. | PICALM | PICALM81 | 480 | MPPSKLVSDDLDSSLANLVGNLGIG | −4.20 | 6.95E−04 |
| | | | | NGTTKNDVNWSQPGEK | | |
| 1399. | PICALM | PICALM82 | 486 | MSDDLDSSLANLVGNLGIGNGTTKN | −2.33 | 1.94E−02 |
| | | | | DVNWSQPGEKKLTGGS | | |
| 1400. | PIK3CA | PIK3CA_17 | 96 | MPFLKVIEPVGNREEKILNREIGFA | −3.47 | 3.04E−03 |
| | | | | IGMPVCEFDMVKDPEV | | |
| 1401. | PIK3CA | PIK3CA18 | 102 | MEPVGNREEKILNREIGFAIGMPVC | −2.08 | 5.47E−02 |
| | | | | EFDMVKDPEVQDFRRN | | |
| 1402. | PIK3CA | PIK3CA29 | 168 | MPNVESSPELPKHIYNKLDKGQIIV | −3.05 | 7.69E−03 |
| | | | | VIWVIVSPNNDKQKYT | | |
| 1403. | PIK3CA | PIK3CA30 | 174 | MPELPKHIYNKLDKGQIIVVIWVIV | −3.20 | 3.86E−03 |
| | | | | SPNNDKQKYTLKINHD | | |
| 1404. | PIK3CA | PIK3CA31 | 180 | MIYNKLDKGQIIVVIWVIVSPNNDK | −3.18 | 6.10E−03 |
| | | | | QKYTLKINHDCVPEQV | | |
| 1405. | PIK3CA | PIK3CA32 | 186 | MKGQIIVVIWVIVSPNNDKQKYTLK | −3.47 | 3.04E−03 |
| | | | | INHDCVPEQVIAEAIR | | |
| 1406. | PIK3CA | PIK3CA38 | 222 | MEAIRKKTRSMLLSSEQLKLCVLEY | −2.72 | 8.70E−03 |
| | | | | QGKYILKVCGCDEYFL | | |
| 1407. | PIK3CA | PIK3CA42 | 246 | MQGKYILKVCGCDEYFLEKYPLSQY | −2.57 | 2.50E−02 |
| | | | | KYIRSCIMLGRMPNLM | | |
| 1408. | PIK3CA | PIK3CA167 | 996 | MLFINLFSMMLGSGMPELQSFDDIA | −2.02 | 5.04E−02 |
| | | | | YIRKTLALDKTEQEAL | | |
| 1409. | PIK3CA | PIK3CA168 | 1002 | MSMMLGSGMPELQSFDDIAYIRKTL | −1.81 | 6.71E−02 |
| | | | | ALDKTEQEALEYFMKQ | | |
| 1410. | PIK3CB | PIK3CB77 | 456 | MIILHSWSSFPDELEEMLNPMGTVQ | −1.87 | 8.02E−02 |
| | | | | TNPYTENATALHVKFP | | |
| 1411. | PIK3CB | PIK3CB78 | 462 | MSSFPDELEEMLNPMGTVQTNPYTE | −2.87 | 6.84E−03 |
| | | | | NATALHVKFPENKKQP | | |
| 1412. | PIK3CB | PIK3CB105 | 624 | MGCLRQMSDEELSQYLLQLVQVLKY | −1.76 | 8.66E−02 |
| | | | | EPFLDCALSRFLLERA | | |
| 1413. | PIK3CB | PIK3CB107 | 636 | MQYLLQLVQVLKYEPFLDCALSRFL | −1.99 | 6.39E−02 |
| | | | | LERALGNRRIGQFLFW | | |
| 1414. | PIK3CB | PIK3CB166 | 990 | MLILRRHGNLFITLFALMLTAGLPE | −1.84 | 8.22E−02 |
| | | | | LTSVKDIQYLKDSLAL | | |
| 1415. | PIK3CG | PIK3CG169 | 1008 | MFQDICVKAYLALRHHTNLLIILFS | −1.92 | 3.97E−02 |
| | | | | MMLMTGMPQLTSKEDI | | |
| 1416. | PINX1 | PINX1_35 | 204 | MTQVERKRGKKINKEATGKDVESYL | −2.86 | 8.64E−03 |
| | | | | QPKAKRHTEGKPERAE | | |
| 1417. | PINX1 | PINX1_36 | 210 | MRGKKINKEATGKDVESYLQPKAKR | −3.03 | 6.61E−03 |
| | | | | HTEGKPERAEAQERVA | | |
| 1418. | PLAG1 | PLAG1_32 | 186 | MCEHCDRRFYTRKDVRRHMVVHTGR | −5.10 | 7.02E−05 |
| | | | | KDFLCQYCAQRFGRKD | | |
| 1419. | PLAG1 | PLAG1_33 | 192 | MRFYTRKDVRRHMVVHTGRKDFLCQ | −2.17 | 2.43E−02 |
| | | | | YCAQRFGRKDHLTRHM | | |
| 1420. | PLCB4 | PLCB4_29 | 168 | MITRTFASGKTEKVIFQALKELGLP | −2.14 | 5.26E−02 |
| | | | | SGKNDEIEPTAFSYEK | | |
| 1421. | PLCB4 | PLCB4_30 | 174 | MSGKTEKVIFQALKELGLPSGKNDE | −1.76 | 9.04E−02 |
| | | | | IEPTAFSYEKFYELTQ | | |
| 1422. | PLCB4 | PLCB4_37 | 216 | MCPRTDIEDLFKKINGDKTDYLTVD | −2.37 | 3.31E−02 |
| | | | | QLVSFLNEHQRDPRLN | | |
| 1423. | PLCB4 | PLCB4_38 | 222 | MEDLFKKINGDKTDYLTVDQLVSFL | −2.53 | 2.16E−02 |
| | | | | NEHQRDPRLNEILFPF | | |
| 1424. | PLCB4 | PLCB4_151 | 900 | MLASGVEAKKGIELIPQVRIEDLKQ | −3.28 | 4.27E−03 |
| | | | | MKAYLKHLKKQQKELN | | |
| 1425. | PLCB4 | PLCB4_152 | 906 | MAKKGIELIPQVRIEDLKQMKAYLK | −3.37 | 3.67E−03 |
| | | | | HLKKQQKELNSLKKKH | | |
| 1426. | PLCB4 | PLCB4_153 | 912 | MLIPQVRIEDLKQMKAYLKHLKKQQ | −3.26 | 4.40E−03 |
| | | | | KELNSLKKKHAKEHST | | |
| 1427. | PLCG1 | PLCG1_136 | 810 | MTFIKSAIIQNVEKQEGGWWRGDYG | −3.04 | 6.48E−03 |
| | | | | GKKQLWFPSNYVEEMV | | |
| 1428. | PLCG1 | PLCG1_137 | 816 | MIIQNVEKQEGGWWRGDYGGKKQLW | −2.97 | 7.28E−03 |
| | | | | FPSNYVEEMVNPVALE | | |
| 1429. | PLK2 | PLK2_23 | 132 | MLHRILHHKHVVQFYHYFEDKENIY | −2.08 | 5.89E−02 |
| | | | | ILLEYCSRRSMAHILK | | |
| 1430. | PLK2 | PLK2_24 | 138 | MHKHVVQFYHYFEDKENIYILLEYC | −1.99 | 6.73E−02 |
| | | | | SRRSMAHILKARKVLT | | |
| 1431. | PLK2 | PLK2_25 | 144 | MFYHYFEDKENIYILLEYCSRRSMA | −1.90 | 7.69E−02 |
| | | | | HILKARKVLTEPEVRY | | |
| 1432. | PLK2 | PLK2_47 | 276 | MRPPFETTNLKETYRCIREARYTMP | −2.23 | 4.46E−02 |
| | | | | SSLLAPAKHLIASMLS | | |
| 1433. | PLK2 | PLK2_48 | 282 | MTNLKETYRCIREARYTMPSSLLAP | −2.78 | 1.86E−02 |
| | | | | AKHLIASMLSKNPEDR | | |
| 1434. | PLK2 | PLK2_73 | 432 | MPAVENKQQIGDAIRMIVRGTLGSC | −3.53 | 2.56E−03 |
| | | | | SSSSECLEDSTMGSVA | | |
| 1435. | PLK2 | PLK2_75 | 444 | MIRMIVRGTLGSCSSSSECLEDSTM | −3.77 | 1.72E−03 |
| | | | | GSVADTVARVLRGCLE | | |
| 1436. | PLXNB2 | PLXNB2262 | 1566 | MPEDSQQDLPGERHALLEEENRVWH | −4.57 | 2.96E−04 |
| | | | | LVRPTDEVDEGKSKRG | | |
| 1437. | PLXNB2 | PLXNB2263 | 1572 | MDLPGERHALLEEENRVWHLVRPTD | −1.94 | 1.02E−02 |
| | | | | EVDEGKSKRGSVKEKE | | |
| 1438. | PLXNB2 | PLXNB2287 | 1716 | MQTFMDACTRTEHKLSRDSPSNKLL | −4.08 | 9.15E−04 |
| | | | | YAKEISTYKKMVEDYY | | |
| 1439. | PLXNB2 | PLXNB2289 | 1728 | MKLSRDSPSNKLLYAKEISTYKKMV | −4.02 | 1.04E−03 |
| | | | | EDYYKGIRQMVQVSDQ | | |
| 1440. | PMS1 | PMS1_18 | 102 | MIAEVLITTRTAADNFSTQYVLDGS | −2.70 | 1.93E−02 |
| | | | | GHILSQKPSHLGQGTT | | |
| 1441. | PMS1 | PMS1_20 | 114 | MDNFSTQYVLDGSGHILSQKPSHLG | −2.37 | 3.21E−02 |
| | | | | QGTTVTALRLFKNLPV | | |
| 1442. | PMS1 | PMS1_21 | 120 | MYVLDGSGHILSQKPSHLGQGTTVT | −2.85 | 1.55E−02 |
| | | | | ALRLFKNLPVRKQFYS | | |
| 1443. | PMS1 | PMS1_29 | 168 | MIKKIQDLLMSFGILKPDLRIVFVH | −2.24 | 4.43E−02 |
| | | | | NKAVIWQKSRVSDHKM | | |
| 1444. | PMS1 | PMS1_31 | 180 | MILKPDLRIVFVHNKAVIWQKSRVS | −2.56 | 2.59E−02 |
| | | | | DHKMALMSVLGTAVMN | | |
| 1445. | PMS1 | PMS1_48 | 282 | MKCLKESTRLYPVFFLKIDVPTADV | −1.97 | 6.02E−02 |
| | | | | DVNLTPDKSQVLLQNK | | |
| 1446. | PMS1 | PMS1_49 | 288 | MTRLYPVFFLKIDVPTADVDVNLTP | −2.69 | 2.19E−02 |
| | | | | DKSQVLLQNKESVLIA | | |
| 1447. | PMS1 | PMS1_50 | 294 | MFFLKIDVPTADVDVNLTPDKSQVL | −2.55 | 1.39E−02 |
| | | | | LQNKESVLIALENLMT | | |
| 1448. | PMS1 | PMS1_51 | 300 | MVPTADVDVNLTPDKSQVLLQNKES | −2.36 | 3.38E−02 |
| | | | | VLIALENLMTTCYGPL | | |
| 1449. | PMS1 | PMS1_98 | 582 | MQDHRPQFLIENPKTSLEDATLQIE | −2.01 | 3.05E−02 |
| | | | | ELWKTLSEEEKLNLFN | | |
| 1450. | PMS1 | PMS1_99 | 588 | MFLIENPKTSLEDATLQIEELWKTL | −2.28 | 2.09E−02 |
| | | | | SEEEKLNLFNGSHYLD | | |
| 1451. | PMS2 | PMS2_52 | 306 | MNEVYHMYNRHQYPFVVLNISVDSE | −3.31 | 4.83E−03 |
| | | | | CVDINVTPDKRQILLQ | | |
| 1452. | PMS2 | PMS2_54 | 318 | MPFVVLNISVDSECVDINVTPDKRQ | −2.43 | 1.66E−02 |
| | | | | ILLQEEKLLLAVLKTS | | |
| 1453. | PMS2 | PMS2_63 | 372 | MSQQPLLDVEGNLIKMHAADLEKPM | −3.32 | 4.00E−03 |
| | | | | VEKQDQSPSLRTGEEK | | |
| 1454. | PMS2 | PMS2_64 | 378 | MDVEGNLIKMHAADLEKPMVEKQDQ | −3.26 | 4.42E−03 |
| | | | | SPSLRTGEEKKDVSIS | | |
| 1455. | PMS2 | PMS2_65 | 384 | MIKMHAADLEKPMVEKQDQSPSLRT | −2.39 | 3.62E−02 |
| | | | | GEEKKDVSISRLREAF | | |
| 1456. | PNN | PNN_10 | 54 | MGRGSLLLRRGFSDSGGGPPAKQRD | −2.27 | 2.10E−02 |
| | | | | LEGAVSRLGGERRTRR | | |
| 1457. | PNN | PNN_11 | 60 | MLRRGFSDSGGGPPAKQRDLEGAVS | −3.20 | 8.41E−03 |
| | | | | RLGGERRTRRESRQES | | |
| 1458. | PNN | PNN_32 | 186 | MQVENERRELFEERRAKQTELRLLE | −2.16 | 5.25E−02 |
| | | | | QKVELAQLQEEWNEHN | | |
| 1459. | PNN | PNN_33 | 192 | MRELFEERRAKQTELRLLEQKVELA | −2.57 | 2.42E−02 |
| | | | | QLQEEWNEHNAKIIKY | | |
| 1460. | PNN | PNN_85 | 504 | MLSLAVLQPTPQVTQEQGHLLPERK | −1.82 | 8.22E−02 |
| | | | | DFPVESVKLTEVPVEP | | |
| 1461. | PNN | PNN_87 | 516 | MTQEQGHLLPERKDFPVESVKLTEV | −1.80 | 8.85E−02 |
| | | | | PVEPVLTVHPESKSKT | | |
| 1462. | PNN | PNN_92 | 546 | MTVHPESKSKTKTRSRSRGRARNKT | −1.86 | 6.60E−02 |
| | | | | SKSRSRSSSSSSSSSS | | |
| 1463. | PNN | PNN_93 | 552 | MKSKTKTRSRSRGRARNKTSKSRSR | −1.88 | 6.20E−02 |
| | | | | SSSSSSSSSSSTSSSS | | |
| 1464. | PNN | PNN_94 | 558 | MRSRSRGRARNKTSKSRSRSSSSSS | −2.27 | 3.58E−02 |
| | | | | SSSSSTSSSSGSSSSS | | |
| 1465. | POLQ | POLQ_25 | 144 | MPFVSVAKEKKYYLQSLFQEVGIKV | −2.07 | 5.93E−02 |
| | | | | DGYMGSTSPSRHFSSL | | |
| 1466. | POLQ | POLQ_26 | 150 | MKEKKYYLQSLFQEVGIKVDGYMGS | −2.01 | 6.55E−02 |
| | | | | TSPSRHFSSLDIAVCT | | |
| 1467. | POLQ | POLQ_79 | 468 | MLDILTYKQMVGRAGRKGVDTVGES | −2.15 | 2.49E−02 |
| | | | | ILICKNSEKSKGIALL | | |
| 1468. | POLQ | POLQ_80 | 474 | MKQMVGRAGRKGVDTVGESILICKN | −2.31 | 2.00E−02 |
| | | | | SEKSKGIALLQGSLKP | | |
| 1469. | POLQ | POLQ_96 | 570 | MQRNQESVOLGAIEACVMWLLENEF | −1.85 | 5.30E−02 |
| | | | | IQSTEASDGTEGKVYH | | |
| 1470. | POLQ | POLQ_97 | 576 | MVQLGAIEACVMWLLENEFIQSTEA | −1.87 | 3.95E−02 |
| | | | | SDGTEGKVYHPTHLGS | | |
| 1471. | POLQ | POLQ_159 | 948 | MIKHSPNIVQDLNKSREHTSSFNCN | −2.17 | 2.43E−02 |
| | | | | FQNGNQEHQTCSIFRA | | |
| 1472. | POLQ | POLQ_160 | 954 | MIVQDLNKSREHTSSFNCNFQNGNQ | −2.28 | 2.59E−02 |
| | | | | EHQTCSIFRARKRASL | | |
| 1473. | POLQ | POLQ_161 | 960 | MKSREHTSSFNCNFQNGNQEHQTCS | −2.47 | 1.57E−02 |
| | | | | IFRARKRASLDINKEK | | |
| 1474. | POLQ | POLQ_182 | 1086 | MKKTEFRNSGPFAKNVSLSGKEKDN | −1.93 | 3.39E−02 |
| | | | | KTSFPLQIKQNCSWNI | | |
| 1475. | POLQ | POLQ_183 | 1092 | MNSGPFAKNVSLSGKEKDNKTSFPL | −1.89 | 3.59E−02 |
| | | | | QIKQNCSWNITLTNDN | | |
| 1476. | POLQ | POLQ_184 | 1098 | MKNVSLSGKEKDNKTSFPLQIKQNC | −1.85 | 3.80E−02 |
| | | | | SWNITLINDNFVEHIV | | |
| 1477. | POLQ | POLQ_265 | 1584 | MVVSPRALELSDPVLDEHHQGDQDG | −2.24 | 2.20E−02 |
| | | | | GDQDERAEKSKLTGTR | | |
| 1478. | POLQ | POLQ_267 | 1596 | MVLDEHHQGDQDGGDQDERAEKSKL | −1.83 | 3.90E−02 |
| | | | | TGTRONHSFIWSGASF | | |
| 1479. | POLQ | POLQ_269 | 1608 | MGDQDERAEKSKLTGTRQNHSFIWS | −3.11 | 5.72E−03 |
| | | | | GASFDLSPGLQRILDK | | |
| 1480. | POLQ | POLQ_270 | 1614 | MAEKSKLTGTRQNHSFIWSGASFDL | −2.90 | 6.51E−03 |
| | | | | SPGLQRILDKVSSPLE | | |
| 1481. | POLQ | POLQ_322 | 1926 | MSASLVPPSLDPSLTLKDRMWYLQS | −2.51 | 2.34E−02 |
| | | | | CLRKESDKECSVVIYD | | |
| 1482. | POLQ | POLQ_323 | 1932 | MPSLDPSLTLKDRMWYLQSCLRKES | −3.79 | 1.66E−03 |
| | | | | DKECSVVIYDFIQSYK | | |
| 1483. | POLQ | POLQ_324 | 1938 | MLTLKDRMWYLQSCLRKESDKECSV | −3.21 | 8.29E−03 |
| | | | | VIYDFIQSYKILLLSC | | |
| 1484. | POLQ | POLQ_353 | 2112 | MIETQAYQLAGHSFSFTSSDDIAEV | −2.98 | 1.18E−02 |
| | | | | LFLELKLPPNREMKNQ | | |
| 1485. | POLQ | POLQ_354 | 2118 | MQLAGHSFSFTSSDDIAEVLFLELK | −3.84 | 1.51E−03 |
| | | | | LPPNREMKNQGSKKTL | | |
| 1486. | POLQ | POLQ_355 | 2124 | MFSFTSSDDIAEVLFLELKLPPNRE | −2.30 | 2.02E−02 |
| | | | | MKNQGSKKTLGSTRRG | | |
| 1487. | POLQ | POLQ_369 | 2208 | MKVVFPLQREKCLNPFLGMERIYPV | −2.58 | 1.93E−02 |
| | | | | SQSHTATGRITFTEPN | | |
| 1488. | POLQ | POLQ_373 | 2232 | MSQSHTATGRITFTEPNIQNVPRDF | −2.87 | 1.56E−02 |
| | | | | EIKMPTLVGESPPSQA | | |
| 1489. | POLQ | POLQ_374 | 2238 | MTGRITFTEPNIQNVPRDFEIKMPT | −2.89 | 1.53E−02 |
| | | | | LVGESPPSQAVGKGLL | | |
| 1490. | POLQ | POLQ_375 | 2244 | MTEPNIQNVPRDFEIKMPTLVGESP | −3.44 | 3.24E−03 |
| | | | | PSQAVGKGLLPMGRGK | | |
| 1491. | POLQ | POLQ_376 | 2250 | MNVPRDFEIKMPTLVGESPPSQAVG | −3.46 | 2.41E−03 |
| | | | | KGLLPMGRGKYKKGFS | | |
| 1492. | POLQ | POLQ_421 | 2520 | MKLQGMFCPIRGGFFILQLHDELLY | −2.75 | 1.02E−02 |
| | | | | EVAEEDVVQVAQIVKN | | |
| 1493. | POLQ | POLQ_422 | 2526 | MCPIRGGFFILQLHDELLYEVAEED | −2.72 | 1.08E−02 |
| | | | | VVQVAQIVKNEMESAV | | |
| 1494. | POLRMT | POLRMT_65 | 384 | MSYPKLHLPLKTLQCLFEKQLHMEL | −2.75 | 1.02E−02 |
| | | | | ASRVCVVSVEKPTLPS | | |
| 1495. | POLRMT | POLRMT66 | 390 | MLPLKTLQCLFEKQLHMELASRVCV | −2.69 | 1.14E−02 |
| | | | | VSVEKPTLPSKEVKHA | | |
| 1496. | POLRMT | POLRMT67 | 396 | MQCLFEKQLHMELASRVCVVSVEKP | −2.73 | 1.06E−02 |
| | | | | TLPSKEVKHARKTLKT | | |
| 1497. | POLRMT | POLRMT69 | 408 | MASRVCVVSVEKPTLPSKEVKHARK | −1.81 | 3.58E−02 |
| | | | | TLKTLRDQWEKALCRA | | |
| 1498. | POLRMT | POLRMT77 | 456 | MEREVYEGRFSLYPFLCLLDEREVV | −5.61 | 1.41E−05 |
| | | | | RMLLQVLQALPAQGES | | |
| 1499. | POLRMT | POLRMT79 | 468 | MPFLCLLDEREVVRMLLQVLQALPA | −3.59 | 8.42E−04 |
| | | | | QGESFITLARELSART | | |
| 1500. | POLRMT | POLRMT_80 | 474 | MDEREVVRMLLQVLQALPAQGESFT | −3.84 | 5.05E−04 |
| | | | | TLARELSARTFSRHVV | | |
| 1501. | POLRMT | POLRMT81 | 480 | MRMLLQVLQALPAQGESFTTLAREL | −2.39 | 5.09E−03 |
| | | | | SARTFSRHVVQRQRVS | | |
| 1502. | POLRMT | POLRMT_82 | 486 | MLQALPAQGESFTTLARELSARTFS | −2.16 | 5.83E−03 |
| | | | | RHVVQRQRVSGQVQAL | | |
| 1503. | PPP6C | PPP6C_14 | 78 | MMGDFVDRGYYSLETFTYLLALKAK | −2.73 | 1.06E−02 |
| | | | | WPDRITLLRGNHESRQ | | |
| 1504. | PPP6C | PPP6C_16 | 90 | METFTYLLALKAKWPDRITLLRGNH | −2.14 | 5.37E−02 |
| | | | | ESRQITQVYGFYDECQ | | |
| 1505. | PPP6C | PPP6C_17 | 96 | MLALKAKWPDRITLLRGNHESRQIT | −3.03 | 6.54E−03 |
| | | | | QVYGFYDECQTKYGNA | | |
| 1506. | PPP6C | PPP6C_18 | 102 | MWPDRITLLRGNHESRQITQVYGFY | −3.12 | 5.68E−03 |
| | | | | DECQTKYGNANAWRYC | | |
| 1507. | PRKAR1A | PRKAR1A38 | 222 | MLWGIDRDSYRRILMGSTLRKRKMY | −3.91 | 1.29E−03 |
| | | | | EEFLSKVSILESLDKW | | |
| 1508. | PRKAR1A | PRKAR1A39 | 228 | MDSYRRILMGSTLRKRKMYEEFLSK | −2.49 | 2.62E−02 |
| | | | | VSILESLDKWERLTVA | | |
| 1509. | PRKAR1A | PRKAR1A42 | 246 | MEEFLSKVSILESLDKWERLTVADA | −3.71 | 1.95E−03 |
| | | | | LEPVQFEDGQKIVVQG | | |
| 1510. | PRKCA | PRKCA_23 | 132 | MDTCDMNVHKQCVINVPSLCGMDHT | −4.37 | 4.73E−04 |
| | | | | EKRGRIYLKAEVADEK | | |
| 1511. | PRKCA | PRKCA_24 | 138 | MVHKQCVINVPSLCGMDHTEKRGRI | −4.35 | 5.20E−04 |
| | | | | YLKAEVADEKLHVTVR | | |
| 1512. | PRKCA | PRKCA_26 | 150 | MCGMDHTEKRGRIYLKAEVADEKLH | −2.33 | 1.95E−02 |
| | | | | VTVRDAKNLIPMDPNG | | |
| 1513. | PRKCA | PRKCA_75 | 444 | MAEISIGLFFLHKRGIIYRDLKLDN | −2.20 | 4.82E−02 |
| | | | | VMLDSEGHIKIADFGM | | |
| 1514. | PRKCA | PRKCA_77 | 456 | MRGIIYRDLKLDNVMLDSEGHIKIA | −2.26 | 2.15E−02 |
| | | | | DFGMCKEHMMDGVTTR | | |
| 1515. | PRKCD | PRKCD_14 | 78 | MEPVSEVTVGVSVLAERCKKNNGKA | −4.73 | 1.96E−04 |
| | | | | EFWLDLQPQAKVLMSV | | |
| 1516. | PRKCD | PRKCD_17 | 96 | MKNNGKAEFWLDLQPQAKVLMSVQY | −2.70 | 1.92E−02 |
| | | | | FLEDVDCKQSMRSEDE | | |
| 1517. | PRKCI | PRKCI_25 | 144 | MCAICTDRIWGLGRQGYKCINCKLL | −3.92 | 1.29E−03 |
| | | | | VHKKCHKLVTIECGRH | | |
| 1518. | PRKCI | PRKCI_29 | 168 | MVHKKCHKLVTIECGRHSLPQEPVM | −1.75 | 7.40E−02 |
| | | | | PMDQSSMHSDHAQTVI | | |
| 1519. | PROX1 | PROX1_39 | 228 | MKEQKREERRQLKQQLEDMQKQLRQ | −2.70 | 1.11E−02 |
| | | | | LQEKFYQIYDSTDSEN | | |
| 1520. | PROX1 | PROX1_40 | 234 | MERRQLKQQLEDMQKQLRQLQEKFY | −2.26 | 2.67E−02 |
| | | | | QIYDSTDSENDEDGNL | | |
| 1521. | PROX1 | PROX1_50 | 294 | MSDNEMCELDPGQFIDRARALIREQ | −3.09 | 8.30E−03 |
| | | | | EMAENKPKREGNNKER | | |
| 1522. | PROX1 | PROX1_51 | 300 | MELDPGQFIDRARALIREQEMAENK | −2.33 | 3.13E−02 |
| | | | | PKREGNNKERDHGPNS | | |
| 1523. | PROX1 | PROX1_52 | 306 | MFIDRARALIREQEMAENKPKREGN | −2.25 | 4.19E−02 |
| | | | | NKERDHGPNSLQPEGK | | |
| 1524. | PROX1 | PROX1_53 | 312 | MALIREQEMAENKPKREGNNKERDH | −2.28 | 3.86E−02 |
| | | | | GPNSLQPEGKHLAETL | | |
| 1525. | PROX1 | PROX1_54 | 318 | MEMAENKPKREGNNKERDHGPNSLQ | −1.96 | 6.37E−02 |
| | | | | PEGKHLAETLKQELNT | | |
| 1526. | PSIP1 | PSIP1_22 | 126 | MKASNEDVTKAVDITTPKAARRGRK | −2.45 | 3.28E−02 |
| | | | | RKAEKQVETEEAGVVT | | |
| 1527. | PSIP1 | PSIP1_23 | 132 | MVTKAVDITTPKAARRGRKRKAEKQ | −2.14 | 5.25E−02 |
| | | | | VETEEAGVVTTATASV | | |
| 1528. | PSIP1 | PSIP1_24 | 138 | MITTPKAARRGRKRKAEKQVETEEA | −2.55 | 2.75E−02 |
| | | | | GVVTTATASVNLKVSP | | |
| 1529. | PSIP1 | PSIP1_69 | 408 | MSQVIMEKSTMLYNKFKNMFLVGEG | −2.25 | 4.50E−02 |
| | | | | DSVITQVLNKSLAEQR | | |
| 1530. | PSIP1 | PSIP1_70 | 414 | MKSTMLYNKFKNMFLVGEGDSVITQ | −2.82 | 9.19E−03 |
| | | | | VLNKSLAEQRQHEEAN | | |
| 1531. | PSIP1 | PSIP1_71 | 420 | MNKFKNMFLVGEGDSVITQVLNKSL | −2.86 | 8.64E−03 |
| | | | | AEQRQHEEANKTKDQG | | |
| 1532. | PTCH1 | PTCH1_10 | 54 | MLETNVEELWVEVGGRVSRELNYTR | −2.68 | 1.14E−02 |
| | | | | QKIGEEAMFNPQLMIQ | | |
| 1533. | PTCH1 | PTCH1_12 | 66 | MGGRVSRELNYTRQKIGEEAMFNPQ | −2.20 | 3.74E−02 |
| | | | | LMIQTPKEEGANVLTT | | |
| 1534. | PTPDC1 | PTPDC11 | 0 | MMAAGVLPQNEQPYSTLVNNSECVA | −2.56 | 1.66E−02 |
| | | | | NMKGNLERPTPKYTKV | | |
| 1535. | PTPDC1 | PTPDC12 | 6 | MPQNEQPYSTLVNNSECVANMKGNL | −2.42 | 2.06E−02 |
| | | | | ERPTPKYTKVGERLRH | | |
| 1536. | PTPRC | PTPRC_38 | 222 | MPSKPTCDEKYANITVDYLYNKETK | −2.02 | 5.99E−02 |
| | | | | LFTAKLNVNENVECGN | | |
| 1537. | PTPRC | PTPRC_39 | 228 | MDEKYANITVDYLYNKETKLFTAKL | −1.86 | 7.99E−02 |
| | | | | NVNENVECGNNTCTNN | | |
| 1538. | PTPRC | PTPRC_40 | 234 | MITVDYLYNKETKLFTAKLNVNENV | −2.02 | 6.11E−02 |
| | | | | ECGNNTCTNNEVHNLT | | |
| 1539. | PTPRC | PTPRC_51 | 300 | MPGVEKFQLHDCTQVEKADTTICLK | −1.96 | 6.93E−02 |
| | | | | WKNIETFTCDTQNITY | | |
| 1540. | PTPRC | PTPRC_52 | 306 | MQLHDCTQVEKADTTICLKWKNIET | −2.24 | 4.35E−02 |
| | | | | FTCDTQNITYRFQCGN | | |
| 1541. | PTPRC | PTPRC_53 | 312 | MQVEKADTTICLKWKNIETFTCDTQ | −1.95 | 7.06E−02 |
| | | | | NITYRFQCGNMIFDNK | | |
| 1542. | PTPRC | PTPRC_54 | 318 | MTTICLKWKNIETFTCDTQNITYRF | −2.59 | 1.98E−02 |
| | | | | QCGNMIFDNKEIKLEN | | |
| 1543. | PTPRC | PTPRC_83 | 492 | MVSMTSDNSMHVKCRPPRDRNGPHE | −1.94 | 3.34E−02 |
| | | | | RYHLEVEAGNTLVRNE | | |
| 1544. | PTPRC | PTPRC_84 | 498 | MNSMHVKCRPPRDRNGPHERYHLEV | −2.70 | 2.11E−02 |
| | | | | EAGNTLVRNESHKNCD | | |
| 1545. | PTPRC | PTPRC_93 | 552 | MKAYFHNGDYPGEPFILHHSTSYNS | −1.82 | 6.53E−02 |
| | | | | KALIAFLAFLIIVTSI | | |
| 1546. | PTPRC | PTPRC_95 | 564 | MPFILHHSTSYNSKALIAFLAFLII | −1.78 | 7.15E−02 |
| | | | | VTSIALLVVLYKIYDL | | |
| 1547. | PTPRD | PTPRD_143 | 852 | MEEGLIWVVGPVLAVVFIICIVIAI | −3.18 | 8.05E−03 |
| | | | | LLYKSSKPDRKRAESD | | |
| 1548. | PTPRD | PTPRD_144 | 858 | MVVGPVLAVVFIICIVIAILLYKSS | −2.04 | 5.50E−02 |
| | | | | KPDRKRAESDSRKSSI | | |
| 1549. | PTTG1 | PTTG1_6 | 30 | MSIKALDGRSQVSTPRFGKTFDAPP | −1.83 | 7.32E−02 |
| | | | | ALPKATRKALGTVNRA | | |
| 1550. | PTTG1 | PTTG1_8 | 42 | MTPRFGKTFDAPPALPKATRKALGT | −2.60 | 2.54E−02 |
| | | | | VNRATEKSVKTKGPLK | | |
| 1551. | PYHIN1 | PYHIN142 | 246 | MNINLKRKFIKKRIIIISNYSKRNS | −2.34 | 3.63E−02 |
| | | | | LLEVNEASSVSEAGPD | | |
| 1552. | PYHIN1 | PYHIN143 | 252 | MKFIKKRIIIISNYSKRNSLLEVNE | −2.32 | 3.87E−02 |
| | | | | ASSVSEAGPDQTFEVP | | |
| 1553. | PYHIN1 | PYHIN144 | 258 | MIIIISNYSKRNSLLEVNEASSVSE | −2.12 | 5.13E−02 |
| | | | | AGPDQTFEVPKDIIRR | | |
| 1554. | PYHIN1 | PYHIN145 | 264 | MYSKRNSLLEVNEASSVSEAGPDQT | −2.15 | 4.56E−02 |
| | | | | FEVPKDIIRRAKKIPK | | |
| 1555. | PYHIN1 | PYHIN158 | 342 | MSMAVVGKGECHNIPCEKGDKLRLF | −3.97 | 1.15E−03 |
| | | | | CFRLRKRENMSKLMSE | | |
| 1556. | PYHIN1 | PYHIN159 | 348 | MKGECHNIPCEKGDKLRLFCFRLRK | −1.78 | 6.46E−02 |
| | | | | RENMSKLMSEMHSFIQ | | |
| 1557. | PYHIN1 | PYHIN160 | 354 | MIPCEKGDKLRLFCFRLRKRENMSK | −3.99 | 1.10E−03 |
| | | | | LMSEMHSFIQIQKNTN | | |
| 1558. | PYHIN1 | PYHIN161 | 360 | MDKLRLFCFRLRKRENMSKLMSEMH | −3.97 | 1.14E−03 |
| | | | | SFIQIQKNTNQRSHDS | | |
| 1559. | RAB8A | RAB8A_16 | 90 | MTNEKSFDNIRNWIRNIEEHASADV | −1.92 | 5.12E−02 |
| | | | | EKMILGNKCDVNDKRQ | | |
| 1560. | RAB8A | RAB8A_17 | 96 | MDNIRNWIRNIEEHASADVEKMILG | −1.96 | 4.92E−02 |
| | | | | NKCDVNDKRQVSKERG | | |
| 1561. | RAF1 | RAF1_13 | 72 | MRNGMSLHDCLMKALKVRGLQPECC | −0.76 | 8.85E−02 |
| | | | | AVFRLLHEHKGKKARL | | |
| 1562. | RAF1 | RAF1_14 | 78 | MHDCLMKALKVRGLQPECCAVFRLL | −1.79 | 9.63E−03 |
| | | | | HEHKGKKARLDWNTDA | | |
| 563. | RAD21 | RAD21_23 | 132 | MFSLNQSRVEEITMREEVGNISILQ | −2.41 | 3.47E−02 |
| | | | | ENDFGDFGMDDREIMR | | |
| 1564. | RAD21 | RAD21_24 | 138 | MRVEEITMREEVGNISILQENDFGD | −2.36 | 3.71E−02 |
| | | | | FGMDDREIMREGSAFE | | |
| 1565. | RAD21 | RAD21_25 | 144 | MMREEVGNISILQENDFGDFGMDDR | −1.89 | 6.72E−02 |
| | | | | EIMREGSAFEDDDMLV | | |
| 1566. | RAD21 | RAD21_69 | 408 | MEADNLDEFLKEFENPEVPREDQQQ | −2.77 | 1.61E−02 |
| | | | | QHQQRDVIDEPIIEEP | | |
| 1567. | RAD21 | RAD21_70 | 414 | MEFLKEFENPEVPREDQQQQHQQRD | −2.09 | 4.28E−02 |
| | | | | VIDEPIIEEPSRLQES | | |
| 1568. | RAD21 | RAD21_72 | 426 | MREDQQQQHQQRDVIDEPIIEEPSR | −2.87 | 1.39E−02 |
| | | | | LQESVMEASRTNIDES | | |
| 1569. | RAD51 | RAD51_6 | 30 | MCGINANDVKKLEEAGFHTVEAVAY | −3.01 | 1.16E−02 |
| | | | | APKKELINIKGISEAK | | |
| 1570. | RAD51 | RAD51_7 | 36 | MDVKKLEEAGFHTVEAVAYAPKKEL | −3.21 | 5.73E−03 |
| | | | | INIKGISEAKADKILA | | |
| 1571. | RAD51 | RAD51_8 | 42 | MEAGFHTVEAVAYAPKKELINIKGI | −2.61 | 2.51E−02 |
| | | | | SEAKADKILAVAERYG | | |
| 1572. | RAD51 | RAD51_9 | 48 | MVEAVAYAPKKELINIKGISEAKAD | −1.76 | 8.57E−02 |
| | | | | KILAVAERYGLSGSDV | | |
| 1573. | RAD51 | RAD51_28 | 162 | MGVAVVITNQVVAQVDGAAMFAADP | −2.80 | 9.46E−03 |
| | | | | KKPIGGNIIAHASTTR | | |
| 1574. | RAD51 | RAD51_31 | 180 | MMFAADPKKPIGGNIIAHASTTRLY | −2.14 | 5.12E−02 |
| | | | | LRKGRGETRICKIYDS | | |
| 1575. | RAD51 | RAD51_32 | 186 | MKKPIGGNIIAHASTTRLYLRKGRG | −2.28 | 4.13E−02 |
| | | | | ETRICKIYDSPCLPEA | | |
| 1576. | RAP1A | RAP1A_23 | 132 | MNLARQWCNCAFLESSAKSKINVNE | −2.35 | 3.66E−02 |
| | | | | IFYDLVRQINRKTPVE | | |
| 1577. | RAP1A | RAP1A_24 | 138 | MCNCAFLESSAKSKINVNEIFYDLV | −2.84 | 8.93E−03 |
| | | | | RQINRKTPVEKKKPKK | | |
| 1578. | RAP1A | RAP1A_25 | 144 | MESSAKSKINVNEIFYDLVRQINRK | −2.86 | 8.62E−03 |
| | | | | TPVEKKKPKKKSCLLL | | |
| 1579. | RARB | RARB_55 | 324 | MICLICGDRQDLEEPTKVDKLQEPL | −2.30 | 2.01E−02 |
| | | | | LEALKIYIRKRRPSKP | | |
| 1580. | RARB | RARB_56 | 330 | MDRQDLEEPTKVDKLQEPLLEALKI | −2.17 | 2.43E−02 |
| | | | | YIRKRRPSKPHMFPKI | | |
| 1581. | RASA1 | RASA1_35 | 204 | MLIRESDRRPGSFVLSFLSQMNVVN | −1.79 | 6.44E−02 |
| | | | | HFRIIAMCGDYYIGGR | | |
| 1582. | RASA1 | RASA1_55 | 324 | MTDEQGLIVEDLVEEVGREEDPHEG | −2.32 | 1.60E−02 |
| | | | | KIWFHGKISKQEAYNL | | |
| 1583. | RASA1 | RASA1_56 | 330 | MIVEDLVEEVGREEDPHEGKIWFHG | −3.30 | 4.14E−03 |
| | | | | KISKQEAYNLLMTVGQ | | |
| 1584. | RASA1 | RASA1_57 | 336 | MEEVGREEDPHEGKIWFHGKISKQE | −3.23 | 3.69E−03 |
| | | | | AYNLLMTVGQVCSFLV | | |
| 1585. | RASA1 | RASA1_58 | 342 | MEDPHEGKIWFHGKISKQEAYNLLM | −3.28 | 4.27E−03 |
| | | | | TVGQVCSFLVRPSDNT | | |
| 1586. | RASA1 | RASA1_59 | 348 | MKIWFHGKISKQEAYNLLMTVGQVC | −3.00 | 6.94E−03 |
| | | | | SFLVRPSDNTPGDYSL | | |
| 1587. | RASA1 | RASA1_81 | 480 | MLLKKGKGKRWKNLYFILEGSDAQL | −2.19 | 2.37E−02 |
| | | | | IYFESEKRATKPKGLI | | |
| 1588. | RASA1 | RASA1_82 | 486 | MGKRWKNLYFILEGSDAQLIYFESE | −2.11 | 2.63E−02 |
| | | | | KRATKPKGLIDLSVCS | | |
| 1589. | RASA1 | RASA1_83 | 492 | MLYFILEGSDAQLIYFESEKRATKP | −2.35 | 1.88E−02 |
| | | | | KGLIDLSVCSVYVVHD | | |
| 1590. | RASA1 | RASA1_87 | 516 | MKGLIDLSVCSVYVVHDSLFGRPNC | −1.80 | 8.78E−02 |
| | | | | FQIVVQHFSEEHYIFY | | |
| 1591. | RASA1 | RASA1_119 | 708 | MRARYSMEKIMPEEEYSEFKELILQ | −1.94 | 6.62E−02 |
| | | | | KELHVVYALSHVCGQD | | |
| 1592. | RASA1 | RASA1_120 | 714 | MEKIMPEEEYSEFKELILQKELHWV | −1.87 | 7.00E−02 |
| | | | | YALSHVCGQDRTLLAS | | |
| 1593. | RASA1 | RASA1_124 | 738 | MYALSHVCGQDRTLLASILLRIFLH | −3.18 | 8.76E−03 |
| | | | | EKLESLLLCTLNDREI | | |
| 1594. | RASA1 | RASA1_140 | 834 | MLEKNEDVNTNLTHLLNILSELVEK | −2.63 | 2.03E−02 |
| | | | | IFMASEILPPTLRYIY | | |
| 1595. | RASA1 | RASA1_141 | 840 | MVNTNLTHLLNILSELVEKIFMASE | −2.37 | 3.52E−02 |
| | | | | ILPPTLRYIYGCLQKS | | |
| 1596. | RASA1 | RASA1_143 | 852 | MSELVEKIFMASEILPPTLRYIYGC | −2.83 | 7.28E−03 |
| | | | | LQKSVQHKWPTNTTMR | | |
| 1597. | RB1 | RB1_30 | 174 | MTQPSSSISTEINSALVLKVSWITF | −1.82 | 7.26E−02 |
| | | | | LLAKGEVLQMEDDLVI | | |
| 1598. | RB1 | RB1_31 | 180 | MISTEINSALVLKVSWITFLLAKGE | −2.43 | 1.66E−02 |
| | | | | VLQMEDDLVISFQLML | | |
| 1599. | RB1 | RB1_32 | 186 | MSALVLKVSWITFLLAKGEVLQMED | −2.24 | 4.42E−02 |
| | | | | DLVISFQLMLCVLDYF | | |
| 1600. | RB1 | RB1_35 | 204 | MVLQMEDDLVISFQLMLCVLDYFIK | −1.98 | 6.67E−02 |
| | | | | LSPPMLLKEPYKTAVI | | |
| 1601. | RB1 | RB1_47 | 276 | MLCKEHECNIDEVKNVYFKNFIPFM | −2.51 | 1.48E−02 |
| | | | | NSLGLVTSNGLPEVEN | | |
| 1602. | RB1 | RB1_48 | 282 | MCNIDEVKNVYFKNFIPFMNSLGLV | −2.11 | 2.64E−02 |
| | | | | TSNGLPEVENLSKRYE | | |
| 1603. | RB1 | RB1_49 | 288 | MKNVYFKNFIPFMNSLGLVTSNGLP | −2.41 | 1.71E−02 |
| | | | | EVENLSKRYEEIYLKN | | |
| 1604. | RB1 | RB1_50 | 294 | MNFIPFMNSLGLVTSNGLPEVENLS | −2.35 | 1.88E−02 |
| | | | | KRYEEIYLKNKDLDAR | | |
| 1605. | RB1 | RB1_56 | 330 | MLDARLFLDHDKTLQTDSIDSFETQ | −2.48 | 1.96E−02 |
| | | | | RTPRKSNLDEEVNVIP | | |
| 1606. | RB1 | RB1_57 | 336 | MLDHDKTLQTDSIDSFETQRTPRKS | −1.82 | 4.80E−02 |
| | | | | NLDEEVNVIPPHTPVR | | |
| 1607. | RB1 | RB1_63 | 372 | MTPVRTVMNTIQQLMMILNSASDQP | −2.06 | 5.30E−02 |
| | | | | SENLISYFNNCTVNPK | | |
| 1608. | RB1 | RB1_64 | 378 | MMNTIQQLMMILNSASDQPSENLIS | −1.80 | 4.08E−02 |
| | | | | YFNNCTVNPKESILKR | | |
| 1609. | RB1 | RB1_65 | 384 | MLMMILNSASDQPSENLISYFNNCT | −1.80 | 4.08E−02 |
| | | | | VNPKESILKRVKDIGY | | |
| 1610. | RB1 | RB1_66 | 390 | MSASDQPSENLISYFNNCTVNPKES | −1.87 | 3.06E−02 |
| | | | | ILKRVKDIGYIFKEKF | | |
| 1611. | RB1 | RB1_67 | 396 | MSENLISYFNNCTVNPKESILKRVK | −2.05 | 2.87E−02 |
| | | | | DIGYIFKEKFAKAVGQ | | |
| 1612. | RB1 | RB1_115 | 684 | MQHTLQNEYELMRDRHLDQIMMCSM | −2.22 | 4.13E−02 |
| | | | | YGICKVKNIDLKFKII | | |
| 1613. | RB1 | RB1_118 | 702 | MIMMCSMYGICKVKNIDLKFKIIVT | −1.87 | 7.64E−02 |
| | | | | AYKDLPHAVQETFKRV | | |
| 1614. | RB1 | RB1_120 | 714 | MKNIDLKFKIIVTAYKDLPHAVQET | −2.32 | 3.48E−02 |
| | | | | FKRVLIKEEEYDSIIV | | |
| 1615. | RB1 | RB1_121 | 720 | MFKIIVTAYKDLPHAVQETFKRVLI | −2.52 | 2.63E−02 |
| | | | | KEEEYDSIIVFYNSVF | | |
| 1616. | RB1 | RB1_122 | 726 | MAYKDLPHAVQETFKRVLIKEEEYD | −2.15 | 4.39E−02 |
| | | | | SIIVFYNSVFMQRLKT | | |
| 1617. | RB1 | RB1_123 | 732 | MHAVQETFKRVLIKEEEYDSIIVFY | −1.95 | 5.62E−02 |
| | | | | NSVFMQRLKTNILQYA | | |
| 1618. | RB1CC1 | RB1CC125 | 144 | MLQHQGWAAIMANLEDCSNSYQKLL | −2.47 | 1.58E−02 |
| | | | | FKFESIYSNYLQSIED | | |
| 1619. | RB1CC1 | RB1CC126 | 150 | MAAIMANLEDCSNSYQKLLFKFESI | −2.30 | 3.67E−02 |
| | | | | YSNYLQSIEDIKLKLT | | |
| 1620. | RB1CC1 | RB1CC150 | 294 | MDTKDGDLPFFNVSLLDWINVQDRP | −2.85 | 8.77E−03 |
| | | | | NDVESLVRKCFDSMSR | | |
| 1621. | RB1CC1 | RB1CC151 | 300 | MLPFFNVSLLDWINVQDRPNDVESL | −2.56 | 2.29E−02 |
| | | | | VRKCFDSMSRLDPRII | | |
| 1622. | RB1CC1 | RB1CC181 | 480 | MERVKIVEALSTVPQMYCLAVVEVV | −2.56 | 2.59E−02 |
| | | | | RRKMFIKHYREWAGAL | | |
| 1623. | RB1CC1 | RB1CC182 | 486 | MEALSTVPQMYCLAVVEVVRRKMFI | −3.68 | 2.06E−03 |
| | | | | KHYREWAGALVKDGKR | | |
| 1624. | RB1CC1 | RB1CC189 | 528 | MEAEKSKRESFGKLFRKSFLRNRLF | −1.76 | 4.29E−02 |
| | | | | RGLDSWPPSFCTQKPR | | |
| 1625. | RB1CC1 | RB1CC1102 | 606 | MPLHQHVLALHNLVKAAQSLDEMSQ | −3.26 | 4.43E−03 |
| | | | | TITDLLSEQKASVSQT | | |
| 1626. | RB1CC1 | RB1CC1186 | 1110 | MIQDNNENYQVGLAELRTLMTIEKD | −2.51 | 1.48E−02 |
| | | | | QCISELISRHEEESNI | | |
| 1627. | RB1CC1 | RB1CC1187 | 1116 | MNYQVGLAELRTLMTIEKDQCISEL | −2.75 | 1.02E−02 |
| | | | | ISRHEEESNILKAELN | | |
| 1628. | RB1CC1 | RB1CC1188 | 1122 | MAELRTLMTIEKDQCISELISRHEE | −2.70 | 1.11E−02 |
| | | | | ESNILKAELNKVTSLH | | |
| 1629. | RB1CC1 | RB1CC1189 | 1128 | MMTIEKDQCISELISRHEEESNILK | −2.71 | 1.09E−02 |
| | | | | AELNKVTSLHNQAFEI | | |
| 1630. | RB1CC1 | RB1CC1217 | 1296 | MEEKAKFLEQLEEQEKRKNEEMQNV | −2.05 | 2.40E−02 |
| | | | | RTSLIAEQQTNFNTVL | | |
| 1631. | RB1CC1 | RB1CC1219 | 1308 | MQEKRKNEEMQNVRTSLIAEQQTNF | −2.25 | 2.17E−02 |
| | | | | NTVLTREKMRKENIIN | | |
| 1632. | RB1CC1 | RB1CC1220 | 1314 | MEEMQNVRTSLIAEQQTNFNTVLTR | −1.93 | 3.42E−02 |
| | | | | EKMRKENIINDLSDKL | | |
| 1633. | RBL1 | RBL1_21 | 120 | MRIERLERNFEVSTVIFKKYEPIFL | −2.14 | 2.53E−02 |
| | | | | DIFQNPYEEPPKLPRS | | |
| 1634. | RBL1 | RBL1_22 | 126 | MRNFEVSTVIFKKYEPIFLDIFQNP | −2.01 | 3.05E−02 |
| | | | | YEEPPKLPRSRKORRI | | |
| 1635. | RBL1 | RBL1_23 | 132 | MTVIFKKYEPIFLDIFQNPYEEPPK | −2.19 | 1.95E−02 |
| | | | | LPRSRKQRRIPCSVKD | | |
| 1636. | RBL1 | RBL1_24 | 138 | MYEPIFLDIFQNPYEEPPKLPRSRK | −2.27 | 2.12E−02 |
| | | | | QRRIPCSVKDLFNFCW | | |
| 1637. | RBL1 | RBL1_149 | 888 | MREVVAYNKNINDDFEMIDCDLEDA | −2.55 | 1.40E−02 |
| | | | | TKTPDCSSGPVKEERG | | |
| 1638. | RBL1 | RBL1_150 | 894 | MNKNINDDFEMIDCDLEDATKTPDC | −2.58 | 1.34E−02 |
| | | | | SSGPVKEERGDLIKFY | | |
| 1639. | RBL1 | RBL1_151 | 900 | MDFEMIDCDLEDATKTPDCSSGPVK | −2.70 | 1.11E−02 |
| | | | | EERGDLIKFYNTIYVG | | |
| 1640. | RBL1 | RBL1_155 | 924 | MEERGDLIKFYNTIYVGRVKSFALK | −3.39 | 4.11E−03 |
| | | | | YDLANQDHMMDAPPLS | | |
| 1641. | RBL1 | RBL1_156 | 930 | MIKFYNTIYVGRVKSFALKYDLANQ | −2.74 | 1.59E−02 |
| | | | | DHMMDAPPLSPFPHIK | | |
| 1642. | RBL2 | RBL2_100 | 594 | MDHLAWKPESPLWEKIRDNENRVPT | −3.07 | 7.45E−03 |
| | | | | CEEVMPPQNLERADEI | | |
| 1643. | RBL2 | RBL2_101 | 600 | MPESPLWEKIRDNENRVPTCEEVMP | −2.25 | 4.40E−02 |
| | | | | PQNLERADEICIAGSP | | |
| 1644. | RBL2 | RBL2_102 | 606 | MEKIRDNENRVPTCEEVMPPQNLER | −2.52 | 2.89E−02 |
| | | | | ADEICIAGSPLTPRRV | | |
| 1645. | RBL2 | RBL2_122 | 726 | MLVTMATATVTANNGQTVTIPVQGI | −2.94 | 7.63E−03 |
| | | | | ANENGGITFFPVQVNV | | |
| 1646. | RBL2 | RBL2_172 | 1026 | MANMDAPPLSPYPFVRTGSPRRIQL | −3.65 | 3.16E−03 |
| | | | | SQNHPVYISPHKNETM | | |
| 1647. | RBL2 | RBL2_174 | 1038 | MFVRTGSPRRIQLSQNHPVYISPHK | −2.01 | 5.64E−02 |
| | | | | NETMLSPREKIFYYFS | | |
| 1648. | RBM10 | RBM10_101 | 600 | MQSQQYLYWDGERRTYVPALEQSAD | −3.07 | 6.09E−03 |
| | | | | GHKETGAPSKEGKEKK | | |
| 1649. | RBM10 | RBM10_102 | 606 | MYWDGERRTYVPALEQSADGHKETG | −2.98 | 7.16E−03 |
| | | | | APSKEGKEKKEKHKTK | | |
| 1650. | RBM15 | RBM15_29 | 168 | MYKTLKISELGSQLSDEAVEDGLFH | −2.17 | 2.43E−02 |
| | | | | EFKRFGDVSVKISHLS | | |
| 1651. | RBM15 | RBM15_31 | 180 | MLSDEAVEDGLFHEFKRFGDVSVKI | −2.09 | 5.72E−02 |
| | | | | SHLSGSGSGDERVAFV | | |
| 1652. | RBM15 | RBM15_32 | 186 | MEDGLFHEFKRFGDVSVKISHLSGS | −3.16 | 8.20E−03 |
| | | | | GSGDERVAFVNFRRPE | | |
| 1653. | RECK | RECK_3 | 12 | MPLLLAVTGVAEVAGGLAPGSAGAL | −1.98 | 1.36E−02 |
| | | | | CCNHSKDNQMCRDVCE | | |
| 1654. | RECK | RECK_4 | 18 | MTGVAEVAGGLAPGSAGALCCNHSK | −3.21 | 4.81E−03 |
| | | | | DNQMCRDVCEQIFSSK | | |
| 1655. | REL | REL_8 | 42 | MNNRTYPSIQIMNYYGKGKVRITLV | −3.27 | 5.19E−03 |
| | | | | TKNDPYKPHPHDLVGK | | |
| 1656. | REL | REL_9 | 48 | MSIQIMNYYGKGKVRITLVTKNDPY | −3.31 | 4.09E−03 |
| | | | | KPHPHDLVGKDCRDGY | | |
| 1657. | REL | REL_10 | 54 | MYYGKGKVRITLVTKNDPYKPHPHD | −2.36 | 3.72E−02 |
| | | | | LVGKDCRDGYYEAEFG | | |
| 1658. | REL | REL_11 | 60 | MVRITLVTKNDPYKPHPHDLVGKDC | −2.40 | 3.47E−02 |
| | | | | RDGYYEAEFGQERRPL | | |
| 1659. | RFC1 | RFC1_91 | 540 | MTIKKETDVFWKSLDFKEQVAEETS | −1.96 | 3.27E−02 |
| | | | | GDSKARNLADDSSENK | | |
| 1660. | RFC1 | RFC1_93 | 552 | MLDFKEQVAEETSGDSKARNLADDS | −2.29 | 2.06E−02 |
| | | | | SENKVENLLWVDKYKP | | |
| 1661. | RFC1 | RFC1_94 | 558 | MVAEETSGDSKARNLADDSSENKVE | −2.03 | 2.96E−02 |
| | | | | NLLWVDKYKPTSLKTI | | |
| 1662. | RFC1 | RFC1_120 | 714 | MHALIMDEVDGMAGNEDRGGIQELI | −2.08 | 5.54E−02 |
| | | | | GLIKHTKIPIICMCND | | |
| 1663. | RFC1 | RFC1_121 | 720 | MEVDGMAGNEDRGGIQELIGLIKHT | −2.51 | 1.49E−02 |
| | | | | KIPIICMCNDRNHPKI | | |
| 1664. | RNASEH2B | RNASEH2B_20 | 114 | MQVVVDNVFPNCILLLKLPGLEKLL | −3.14 | 5.42E−03 |
| | | | | HHVTEEKGNPEIDNKK | | |
| 1665. | RNASEH2B | RNASEH2B_21 | 120 | MVFPNCILLLKLPGLEKLLHHVTEE | −2.91 | 6.43E−03 |
| | | | | KGNPEIDNKKYYKYSK | | |
| 1666. | RNASEH2B | RNASEH2B_22 | 126 | MLLLKLPGLEKLLHHVTEEKGNPEI | −2.91 | 8.00E−03 |
| | | | | DNKKYYKYSKEKTLKW | | |
| 1667. | RNASEH2B | RNASEH2B_23 | 132 | MGLEKLLHHVTEEKGNPEIDNKKYY | −3.06 | 6.26E−03 |
| | | | | KYSKEKTLKWLEKKVN | | |
| 1668. | RNASEH2B | RNASEH2B_34 | 198 | MQASTDKEKDYIRYAHGLISDYIPK | −2.25 | 2.17E−02 |
| | | | | ELSDDLSKYLKLPEPS | | |
| 1669. | RNASEH2B | RNASEH2B_36 | 210 | MYAHGLISDYIPKELSDDLSKYLKL | −2.69 | 1.13E−02 |
| | | | | PEPSASLPNPPSKKIK | | |
| 1670. | RNASEH2B | RNASEH2B_37 | 216 | MSDYIPKELSDDLSKYLKLPEPSAS | −1.76 | 8.90E−02 |
| | | | | LPNPPSKKIKLSDEPV | | |
| 1671. | RNASEH2B | RNASEH2B_46 | 270 | MKTEKKNSKMTAAQKALAKVDKSGM | −2.61 | 1.28E−02 |
| | | | | KSIDTFFGVKKKLERF | | |
| 1672. | RNASEH2B | RNASEH2B_47 | 276 | MSKMTAAQKALAKVDKSGMKSIDTF | −2.57 | 1.36E−02 |
| | | | | FGVKKKLERFETLKIK | | |
| 1673. | ROS1 | ROS1_61 | 360 | MIFYRGSGLISSISIDWLYQRMYFI | −1.96 | 5.29E−02 |
| | | | | MDELVCVCDLENCSNI | | |
| 1674. | ROS1 | ROS1_62 | 366 | MGLISSISIDWLYQRMYFIMDELVC | −2.26 | 3.79E−02 |
| | | | | VCDLENCSNIEEITPP | | |
| 1675. | ROS1 | ROS1_134 | 798 | MGYLYWTTLYSVESTRLNGESSLVL | −4.04 | 9.93E−04 |
| | | | | QTQPWFSGKKVIALTL | | |
| 1676. | ROS1 | ROS1_135 | 804 | MTLYSVESTRLNGESSLVLQTQPWF | −3.12 | 9.24E−03 |
| | | | | SGKKVIALTLDLSDGL | | |
| 1677. | ROS1 | ROS1_168 | 1002 | MFTVEGLEPYALFNLSVTPYTYWGK | −1.89 | 5.24E−02 |
| | | | | GPKTSLSLRAPETVPS | | |
| 1678. | ROS1 | ROS1_171 | 1020 | MYTYWGKGPKTSLSLRAPETVPSAP | −1.98 | 6.84E−02 |
| | | | | ENPRIFILPSGKCCNK | | |
| 1679. | ROS1 | ROS1_300 | 1794 | MNLQNQNLRWKMTFNGSCSSVCTWK | −1.76 | 5.98E−02 |
| | | | | SKNLKGIFQFRVVAAN | | |
| 1680. | ROS1 | ROS1_301 | 1800 | MLRWKMTFNGSCSSVCTWKSKNLKG | −2.80 | 1.48E−02 |
| | | | | IFQFRVVAANNLGFGE | | |
| 1681. | RPL22 | RPL22_8 | 42 | MLQERIKVNGKAGNLGGGVVTIERS | −2.06 | 5.96E−02 |
| | | | | KSKITVTSEVPFSKRY | | |
| 1682. | RPL22 | RPL22_10 | 54 | MNLGGGVVTIERSKSKITVTSEVPF | −2.05 | 6.18E−02 |
| | | | | SKRYLKYLTKKYLKKN | | |
| 1683. | RPS6KA3 | RPS6KA353 | 312 | MAGPDGVEEIKRHSFFSTIDWNKLY | −2.38 | 1.81E−02 |
| | | | | RREIHPPFKPATGRPE | | |
| 1684. | RPS6KA3 | RPS6KA354 | 318 | MEEIKRHSFFSTIDWNKLYRREIHP | −2.39 | 1.77E−02 |
| | | | | PFKPATGRPEDTFYFD | | |
| 1685. | RPS6KA3 | RPS6KA384 | 498 | MGELLDKILRQKFFSEREASAVLFT | −2.12 | 5.15E−02 |
| | | | | ITKTVEYLHAQGVVHR | | |
| 1686. | RPS6KA3 | RPS6KA385 | 504 | MILRQKFFSEREASAVLFTITKTVE | −2.46 | 3.11E−02 |
| | | | | YLHAQGVVHRDLKPSN | | |
| 1687. | RRAS2 | RRAS2_20 | 114 | MKDRDEFPMILIGNKADLDHQRQVT | −2.47 | 1.57E−02 |
| | | | | QEEGQQLARQLKVTYM | | |
| 1688. | RRAS2 | RRAS2_22 | 126 | MNKADLDHQRQVTQEEGQQLARQLK | −2.92 | 7.90E−03 |
| | | | | VTYMEASAKIRMNVDQ | | |
| 1689. | RUNX1T1 | RUNX1T162 | 366 | MKGGGSSSSHSRQQSPVNPDPVALD | −2.02 | 7.50E−03 |
| | | | | AHREFLHRPASGYVPE | | |
| 1690. | RUNX1T1 | RUNX1T165 | 384 | MDPVALDAHREFLHRPASGYVPEEI | −2.08 | 2.91E−02 |
| | | | | WKKAEEAVNEVKRQAM | | |
| 1691. | RUNX1T1 | RUNX1T171 | 420 | MRQAMTELQKAVSEAERKAHDMITT | −2.29 | 2.06E−02 |
| | | | | ERAKMERTVAEAKRQA | | |
| 1692. | RUNX1T1 | RUNX1T1_74 | 438 | MHDMITTERAKMERTVAEAKRQAAE | −2.41 | 6.28E−03 |
| | | | | DALAVINQQEDSSESC | | |
| 1693. | RUNX1T1 | RUNX1T175 | 444 | MERAKMERTVAEAKRQAAEDALAVI | −1.87 | 4.15E−02 |
| | | | | NQQEDSSESCWNCGRK | | |
| 1694. | RUNX1T1 | RUNX1T1_76 | 450 | MRTVAEAKRQAAEDALAVINQQEDS | −1.81 | 4.60E−02 |
| | | | | SESCWNCGRKASETCS | | |
| 1695. | RUNX1T1 | RUNX1T177 | 456 | MKRQAAEDALAVINQQEDSSESCWN | −2.63 | 1.17E−02 |
| | | | | CGRKASETCSGCNTAR | | |
| 1696. | SCAF4 | SCAF4_164 | 978 | MQPQQFRNDNRQQFNSGRDQERFGR | −2.10 | 5.75E−02 |
| | | | | RSFGNRVENDRERYGN | | |
| 1697. | SCAF4 | SCAF4_165 | 984 | MNDNRQQFNSGRDQERFGRRSFGNR | −1.86 | 7.94E−02 |
| | | | | VENDRERYGNRNDDRD | | |
| 1698. | SCAF4 | SCAF4_166 | 990 | MFNSGRDQERFGRRSFGNRVENDRE | −2.02 | 6.40E−02 |
| | | | | RYGNRNDDRDNSNRDR | | |
| 1699. | SCAF4 | SCAF4_168 | 1002 | MRSFGNRVENDRERYGNRNDDRDNS | −3.04 | 6.48E−03 |
| | | | | NRDRREWGRRSPDRDR | | |
| 1700. | SCAF4 | SCAF4_169 | 1008 | MVENDRERYGNRNDDRDNSNRDRRE | −2.89 | 6.64E−03 |
| | | | | WGRRSPDRDRHRDLEE | | |
| 1701. | SDHA | SDHA_93 | 552 | MDRGMVWNTDLVETLELQNLMLCAL | −4.00 | 1.08E−03 |
| | | | | QTIYGAEARKESRGAH | | |
| 1702. | SDHA | SDHA_94 | 558 | MNTDLVETLELQNLMLCALQTIYGA | −4.01 | 1.06E−03 |
| | | | | EARKESRGAHAREDYK | | |
| 1703. | SDHA | SDHA_96 | 570 | MLMLCALQTIYGAEARKESRGAHAR | −3.31 | 4.07E−03 |
| | | | | EDYKVRIDEYDYSKPI | | |
| 1704. | SDHA | SDHA_97 | 576 | MQTIYGAEARKESRGAHAREDYKVR | −4.42 | 4.20E−04 |
| | | | | IDEYDYSKPIQGQQKK | | |
| 1705. | SEC31A | SEC31A20 | 114 | MIAQNDKHTGPVRALDVNIFQTNLV | −3.27 | 4.33E−03 |
| | | | | ASGANESEIYIWDLNN | | |
| 1706. | SEC31A | SEC31A21 | 120 | MHTGPVRALDVNIFQTNLVASGANE | −1.79 | 8.68E−02 |
| | | | | SEIYIWDLNNFATPMT | | |
| 1707. | SEC31A | SEC31A23 | 132 | MFQTNLVASGANESEIYIWDLNNFA | −2.76 | 1.86E−02 |
| | | | | TPMTPGAKTQPPEDIS | | |
| 1708. | SEC31A | SEC31A28 | 162 | MAKTQPPEDISCIAWNRQVQHILAS | −2.03 | 6.32E−02 |
| | | | | ASPSGRATVWDLRKNE | | |
| 1709. | SEC31A | SEC31A100 | 594 | MMADAIILAIAGGQELLARTQKKYF | −3.34 | 5.94E−03 |
| | | | | AKSQSKITRLITAVVM | | |
| 1710. | SEC31A | SEC31A101 | 600 | MLAIAGGQELLARTQKKYFAKSQSK | −2.37 | 3.40E−02 |
| | | | | ITRLITAVVMKNWKEI | | |
| 1711. | SETD2 | SETD2_154 | 918 | MQDRKKVRVEVEQGETSVPPGSALV | −2.17 | 2.43E−02 |
| | | | | GPSCVMDDFRDPQRWK | | |
| 1712. | SETD2 | SETD2_157 | 936 | MPGSALVGPSCVMDDFRDPQRWKEC | −2.20 | 2.34E−02 |
| | | | | AKQGKMPCYFDLIEEN | | |
| 1713. | SETD2 | SETD2_173 | 1032 | MGDYCSNRRFQRKQHADVEVILTEK | −2.20 | 4.81E−02 |
| | | | | KGWGLRAAKDLPSNTF | | |
| 1714. | SETD2 | SETD2_174 | 1038 | MRRFQRKQHADVEVILTEKKGWGLR | −3.30 | 4.11E−03 |
| | | | | AAKDLPSNTFVLEYCG | | |
| 1715. | SETD2 | SETD2_246 | 1470 | MINEETPSQDEEEGVSDVESERSQE | −2.14 | 4.71E−02 |
| | | | | QPDKTVDISDLATKLL | | |
| 1716. | SETD2 | SETD2_247 | 1476 | MSQDEEEGVSDVESERSQEQPDKTV | −3.51 | 2.82E−03 |
| | | | | DISDLATKLLDSWKDL | | |
| 1717. | SETD2 | SETD2_248 | 1482 | MGVSDVESERSQEQPDKTVDISDLA | −3.41 | 3.39E−03 |
| | | | | TKLLDSWKDLKEVYRI | | |
| 1718. | SETD2 | SETD2_251 | 1500 | MDISDLATKLLDSWKDLKEVYRIPK | −3.44 | 3.21E−03 |
| | | | | KSQTEKENTTTERGRD | | |
| 1719. | SETD2 | SETD2_253 | 1512 | MWKDLKEVYRIPKKSQTEKENTTTE | −3.50 | 2.88E−03 |
| | | | | RGRDAVGFRDQTPAPK | | |
| 1720. | SETD2 | SETD2_254 | 1518 | MVYRIPKKSQTEKENTITERGRDAV | −2.53 | 2.80E−02 |
| | | | | GFRDQTPAPKTPNRSR | | |
| 1721. | SF3B1 | SF3B1_83 | 492 | MKERKIMKLLLKIKNGTPPMRKAAL | −2.49 | 3.06E−02 |
| | | | | RQITDKAREFGAGPLF | | |
| 1722. | SF3B1 | SF3B1_84 | 498 | MKLLLKIKNGTPPMRKAALRQITDK | −1.93 | 7.14E−02 |
| | | | | AREFGAGPLFNQILPL | | |
| 1723. | SF3B1 | SF3B1_124 | 738 | MRGKGLAAFLKAIGYLIPLMDAEYA | −2.78 | 1.33E−02 |
| | | | | NYYTREVMLILIREFQ | | |
| 1724. | SF3B1 | SF3B1_125 | 744 | MAFLKAIGYLIPLMDAEYANYYTRE | −3.47 | 4.70E−03 |
| | | | | VMLILIREFQSPDEEM | | |
| 1725. | SH3GL1 | SH3GL1_31 | 180 | MEELRQALEKFEESKEVAETSMHNL | −2.57 | 2.20E−02 |
| | | | | LETDIEQVSQLSALVD | | |
| 1726. | SH3GL1 | SH3GL134 | 198 | MTSMHNLLETDIEQVSQLSALVDAQ | −4.08 | 2.86E−06 |
| | | | | LDYHRQAVQILDELAE | | |
| 1727. | SLC5A8 | SLC5A882 | 486 | MTNLMTTTEMPFTTSVFQIYNVQRT | −1.89 | 7.68E−02 |
| | | | | PLMDNWYSLSYLYFST | | |
| 1728. | SLC5A8 | SLC5A884 | 498 | MTSVFQIYNVQRTPLMDNWYSLSYL | −2.30 | 4.14E−02 |
| | | | | YFSTVGTLVTLLVGIL | | |
| 1729. | SMAD2 | SMAD27 | 36 | MEEKWCEKAVKSLVKKLKKTGRLDE | −2.42 | 3.41E−02 |
| | | | | LEKAITTQNCNTKCVT | | |
| 1730. | SMAD2 | SMAD28 | 42 | MKAVKSLVKKLKKTGRLDELEKAIT | −2.85 | 1.59E−02 |
| | | | | TQNCNTKCVTIPSTCS | | |
| 1731. | SMAD4 | SMAD453 | 312 | MPISNHPAPEYWCSIAYFEMDVQVG | −3.83 | 1.54E−03 |
| | | | | ETFKVPSSCPIVTVDG | | |
| 1732. | SMAD4 | SMAD454 | 318 | MAPEYWCSIAYFEMDVQVGETFKVP | −2.73 | 1.88E−02 |
| | | | | SSCPIVTVDGYVDPSG | | |
| 1733. | SMAD4 | SMAD455 | 324 | MSIAYFEMDVQVGETFKVPSSCPIV | −2.69 | 1.91E−02 |
| | | | | TVDGYVDPSGGDRFCL | | |
| 1734. | SMAD4 | SMAD461 | 360 | MRFCLGQLSNVHRTEAIERARLHIG | −3.72 | 1.91E−03 |
| | | | | KGVQLECKGEGDVWVR | | |
| 1735. | SMAD4 | SMAD462 | 366 | MLSNVHRTEAIERARLHIGKGVQLE | −1.77 | 2.96E−02 |
| | | | | CKGEGDVWVRCLSDHA | | |
| 1736. | SMAD4 | SMAD464 | 378 | MARLHIGKGVQLECKGEGDVWVRCL | −1.82 | 3.98E−02 |
| | | | | SDHAVFVQSYYLDREA | | |
| 1737. | SMARCA1 | SMARCA1_8 | 42 | MATEATAATEKGEKKKEKNVSSFQL | −1.81 | 8.22E−02 |
| | | | | KLAAKAPKSEKEMDPE | | |
| 1738. | SMARCA1 | SMARCA1_10 | 54 | MKKKEKNVSSFQLKLAAKAPKSEKE | −1.80 | 8.61E−02 |
| | | | | MDPEYEEKMKADRAKR | | |
| 1739. | SMARCA1 | SMARCA1_46 | 270 | MAAFIRDEMMPGEWDVCVTSYEMVI | −1.86 | 3.77E−02 |
| | | | | KEKSVFKKFHWRYLVI | | |
| 1740. | SMARCA1 | SMARCA1_50 | 294 | MKEKSVFKKFHWRYLVIDEAHRIKN | −2.03 | 6.08E−02 |
| | | | | EKSKLSEIVREFKSTN | | |
| 1741. | SMARCA1 | SMARCA1_51 | 300 | MKKFHWRYLVIDEAHRIKNEKSKLS | −1.77 | 8.95E−02 |
| | | | | EIVREFKSTNRLLLTG | | |
| 1742. | SMARCA1 | SMARCA1_53 | 312 | MAHRIKNEKSKLSEIVREFKSTNRL | −2.01 | 6.47E−02 |
| | | | | LLTGTPLQNNLHELWA | | |
| 1743. | SMARCA1 | SMARCA1_78 | 462 | MPYLFDGAEPGPPYTTDEHIVSNSG | −3.06 | 6.26E−03 |
| | | | | KMVVLDKLLAKLKEQG | | |
| 1744. | SMARCA1 | SMARCA1_81 | 480 | MIVSNSGKMVVLDKLLAKLKEQGSR | −1.78 | 8.96E−02 |
| | | | | VLIFSQMTRLLDILED | | |
| 1745. | SMARCA4 | SMARCA4_98 | 582 | MKKKKKKAENAEGQTPAIGPDGEPL | −2.98 | 5.70E−03 |
| | | | | DETSQMSDLPVKVIHV | | |
| 1746. | SMARCA4 | SMARCA4_99 | 588 | MAENAEGQTPAIGPDGEPLDETSQM | −3.29 | 3.30E−03 |
| | | | | SDLPVKVIHVESGKIL | | |
| 1747. | SMARCA4 | SMARCA4_101 | 600 | MPDGEPLDETSQMSDLPVKVIHVES | −5.12 | 6.67E−05 |
| | | | | GKILTGTDAPKAGQLE | | |
| 1748. | SMARCB1 | SMARCB1_13 | 72 | MTTLATSVTLLKASEVEEILDGNDE | −2.04 | 2.90E−02 |
| | | | | KYKAVSISTEPPTYLR | | |
| 1749. | SMARCB1 | SMARCB1_14 | 78 | MVTLLKASEVEEILDGNDEKYKAVS | −1.81 | 8.08E−02 |
| | | | | ISTEPPTYLREQKAKR | | |
| 1750. | SMC1A | SMC1A47 | 276 | MQQQIEKEIKEKDSELNQKRPQYIK | −2.60 | 1.30E−02 |
| | | | | AKENTSHKIKKLEAAK | | |
| 1751. | SMC1A | SMC1A48 | 282 | MEIKEKDSELNQKRPQYIKAKENTS | −2.44 | 1.35E−02 |
| | | | | HKIKKLEAAKKSLQNA | | |
| 1752. | SMC1A | SMC1A49 | 288 | MSELNQKRPQYIKAKENTSHKIKKL | −2.57 | 1.35E−02 |
| | | | | EAAKKSLQNAQKHYKK | | |
| 1753. | SMC1A | SMC1A50 | 294 | MRPQYIKAKENTSHKIKKLEAAKKS | −2.53 | 1.44E−02 |
| | | | | LQNAQKHYKKRKGDMD | | |
| 1754. | SMC3 | SMC3_60 | 354 | MKEKEERGIARLAQATQERTDLYAK | −3.00 | 6.93E−03 |
| | | | | QGRGSQFTSKEERDKW | | |
| 1755. | SMC3 | SMC3_62 | 366 | MQATQERTDLYAKQGRGSQFTSKEE | −2.28 | 2.09E−02 |
| | | | | RDKWIKKELKSLDQAI | | |
| 1756. | SMC3 | SMC3_63 | 372 | MTDLYAKQGRGSQFTSKEERDKWIK | −2.43 | 1.66E−02 |
| | | | | KELKSLDQAINDKKRQ | | |
| 1757. | SMC3 | SMC3_64 | 378 | MQGRGSQFTSKEERDKWIKKELKSL | −2.01 | 5.18E−02 |
| | | | | DQAINDKKRQIAAIHK | | |
| 1758. | SMC3 | SMC3_77 | 456 | MEVKNKKDELQSERNYLWREENAEQ | −2.17 | 2.43E−02 |
| | | | | QALAAKREDLEKKQQL | | |
| 1759. | SMC3 | SMC3_78 | 462 | MDELQSERNYLWREENAEQQALAAK | −1.85 | 7.37E−02 |
| | | | | REDLEKKQQLLRAATG | | |
| 1760. | SMC3 | SMC3_79 | 468 | MRNYLWREENAEQQALAAKREDLEK | −2.48 | 1.55E−02 |
| | | | | KQQLLRAATGKAILNG | | |
| 1761. | SMC3 | SMC3_113 | 672 | MKSRLELQKDVRKAEEELGELEAKL | −2.08 | 5.86E−02 |
| | | | | NENLRRNIERINNEID | | |
| 1762. | SMC3 | SMC3_114 | 678 | MQKDVRKAEEELGELEAKLNENLRR | −1.87 | 7.96E−02 |
| | | | | NIERINNEIDQLMNQM | | |
| 1763. | SMC3 | SMC3_122 | 726 | MKFKASRDSILSEMKMLKEKRQQSE | −3.04 | 1.06E−02 |
| | | | | KTFMPKQRSLQSLEAS | | |
| 1764. | SMC3 | SMC3_123 | 732 | MDSILSEMKMLKEKRQQSEKTFMPK | −1.87 | 5.14E−02 |
| | | | | QRSLQSLEASLHAMES | | |
| 1765. | SMC3 | SMC3_124 | 738 | MMKMLKEKRQQSEKTFMPKQRSLQS | −3.09 | 9.61E−03 |
| | | | | LEASLHAMESTRESLK | | |
| 1766. | SMC3 | SMC3_125 | 744 | MKRQQSEKTFMPKQRSLQSLEASLH | −2.94 | 1.23E−02 |
| | | | | AMESTRESLKAELGTD | | |
| 1767. | SMC3 | SMC3_147 | 876 | MMARSEDLDNSIDKTEAGIKELQKS | −1.88 | 3.63E−02 |
| | | | | MERWKNMEKEHMDAIN | | |
| 1768. | SMC3 | SMC3_148 | 882 | MLDNSIDKTEAGIKELQKSMERWKN | −2.11 | 2.63E−02 |
| | | | | MEKEHMDAINHDTKEL | | |
| 1769. | SMC3 | SMC3_149 | 888 | MKTEAGIKELQKSMERWKNMEKEHM | −2.05 | 2.87E−02 |
| | | | | DAINHDTKELEKMTNR | | |
| 1770. | SMC3 | SMC3_180 | 1074 | MGESERGSGSQSSVPSVDQFTGVGI | −2.84 | 1.27E−02 |
| | | | | RVSFTGKQGEMREMQQ | | |
| 1771. | SMC3 | SMC3_181 | 1080 | MSGSQSSVPSVDQFTGVGIRVSFTG | −2.08 | 5.90E−02 |
| | | | | KQGEMREMQQLSGGQK | | |
| 1772. | SOS1 | SOS1_11 | 60 | MQAQPRSASDVEERVQKSFPHPIDK | −2.67 | 2.21E−02 |
| | | | | WAIADAQSAIEKRKRR | | |
| 1773. | SOS1 | SOS1_13 | 72 | MRVQKSFPHPIDKWAIADAQSAIEK | −2.54 | 2.80E−02 |
| | | | | RKRRNPLSLPVEKIHP | | |
| 1774. | SOS1 | SOS1_18 | 102 | MLSLPVEKIHPLLKEVLGYKIDHQV | −2.52 | 2.89E−02 |
| | | | | SVYIVAVLEYISADIL | | |
| 1775. | SOS1 | SOS1_19 | 108 | MKIHPLLKEVLGYKIDHQVSVYIVA | −2.47 | 3.17E−02 |
| | | | | VLEYISADILKLVGNY | | |
| 1776. | SOS1 | SOS1_20 | 114 | MKEVLGYKIDHQVSVYIVAVLEYIS | −2.89 | 1.53E−02 |
| | | | | ADILKLVGNYVRNIRH | | |
| 1777. | SOS1 | SOS1_21 | 120 | MKIDHQVSVYIVAVLEYISADILKL | −2.56 | 2.64E−02 |
| | | | | VGNYVRNIRHYEITKQ | | |
| 1778. | SOS1 | SOS1_22 | 126 | MSVYIVAVLEYISADILKLVGNYVR | −3.05 | 6.36E−03 |
| | | | | NIRHYEITKQDIKVAM | | |
| 1779. | SOS1 | SOS1_65 | 384 | MSGMEKICSKSLAKRRLSESACRFY | −1.95 | 7.24E−02 |
| | | | | SQQMKGKQLAIKKMNE | | |
| 1780. | SOS1 | SOS1_66 | 390 | MCSKSLAKRRLSESACRFYSQQMKG | −2.73 | 1.06E−02 |
| | | | | KQLAIKKMNEIQKNID | | |
| 1781. | SOS1 | SOS1_117 | 696 | MVEHHFYDFERDAYLLQRMEEFIGT | −2.47 | 1.58E−02 |
| | | | | VRGKAMKKWVESITKI | | |
| 1782. | SOS1 | SOS1_118 | 702 | MDFERDAYLLQRMEEFIGTVRGKAM | −2.27 | 2.12E−02 |
| | | | | KKWVESITKIIQRKKI | | |
| 1783. | SOS1 | SOS1_119 | 708 | MYLLQRMEEFIGTVRGKAMKKWVES | −2.45 | 1.63E−02 |
| | | | | ITKIIQRKKIARDNGP | | |
| 1784. | SOS1 | SOS1_120 | 714 | MEEFIGTVRGKAMKKWVESITKIIQ | −2.48 | 1.55E−02 |
| | | | | RKKIARDNGPGHNITF | | |
| 1785. | SOS1 | SOS1_123 | 732 | MITKIIQRKKIARDNGPGHNITFQS | −1.90 | 7.53E−02 |
| | | | | SPPTVEWHISRPGHIE | | |
| 1786. | SOS1 | SOS1_146 | 870 | MVLEVVSAMNSSPVYRLDHTFEQIP | −2.06 | 6.12E−02 |
| | | | | SRQKKILEEAHELSED | | |
| 1787. | SOS1 | SOS1_147 | 876 | MAMNSSPVYRLDHTFEQIPSRQKKI | −2.64 | 1.81E−02 |
| | | | | LEEAHELSEDHYKKYL | | |
| 1788. | SOX17 | SOX17_16 | 90 | MPDLHNAELSKMLGKSWKALTLAEK | −3.64 | 2.24E−03 |
| | | | | RPFVEEAERLRVQHMQ | | |
| 1789. | SOX17 | SOX17_17 | 96 | MELSKMLGKSWKALTLAEKRPFVEE | −4.49 | 3.57E−04 |
| | | | | AERLRVQHMQDHPNYK | | |
| 1790. | SOX17 | SOX17_18 | 102 | MGKSWKALTLAEKRPFVEEAERLRV | −3.71 | 1.92E−03 |
| | | | | QHMQDHPNYKYRPRRR | | |
| 1791. | SOX17 | SOX17_19 | 108 | MLTLAEKRPFVEEAERLRVQHMQDH | −3.48 | 3.01E−03 |
| | | | | PNYKYRPRRRKQVKRL | | |
| 1792. | SPECC1 | SPECC1_7 | 36 | MERSVPRGPSNPRKSVSSPTSSNTP | −3.47 | 3.07E−03 |
| | | | | TPTKHLRTPSTKPKQE | | |
| 1793. | SPECC1 | SPECC18 | 42 | MGPSNPRKSVSSPTSSNTPTPTKHL | −4.55 | 3.10E−04 |
| | | | | RTPSTKPKQENEGGEK | | |
| 1794. | SPECC1 | SPECC19 | 48 | MKSVSSPTSSNTPTPTKHLRTPSTK | −3.37 | 5.31E−03 |
| | | | | PKQENEGGEKAALESQ | | |
| 1795. | SPECC1 | SPECC119 | 108 | MELKKYKEKRTLNAEGTDALGPNVD | −2.55 | 2.73E−02 |
| | | | | GTSVSPGDTEPMIRAL | | |
| 1796. | SPECC1 | SPECC120 | 114 | MEKRTLNAEGTDALGPNVDGTSVSP | −2.18 | 4.96E−02 |
| | | | | GDTEPMIRALEEKNKN | | |
| 1797. | SPECC1 | SPECC131 | 180 | MNSEGAASHTGDSSCPTSITQESSF | −2.47 | 1.58E−02 |
| | | | | GSPTGNQLSSDIDEYK | | |
| 1798. | SPECC1 | SPECC133 | 192 | MSCPTSITQESSFGSPTGNQLSSDI | −1.95 | 6.43E−02 |
| | | | | DEYKKNIHGNALRTSG | | |
| 1799. | SPECC1 | SPECC142 | 246 | MASDFEHITAETPSRPLSSTSNPFK | −2.34 | 2.08E−02 |
| | | | | SSKCSTAGSSPNSVSE | | |
| 1800. | SPECC1 | SPECC144 | 258 | MSRPLSSTSNPFKSSKCSTAGSSPN | −2.46 | 1.59E−02 |
| | | | | SVSELSLASLTEKIQK | | |
| 1801. | SPECC1 | SPECC177 | 456 | MEECRVTLEGLKMENGSLKSHLQGE | −1.91 | 7.16E−02 |
| | | | | KQKATEASAVEQTAES | | |
| 1802. | SPECC1 | SPECC178 | 462 | MLEGLKMENGSLKSHLQGEKQKATE | −1.97 | 6.64E−02 |
| | | | | ASAVEQTAESCEVQEM | | |
| 1803. | SPECC1 | SPECC1110 | 654 | MNDIKCEAQQELRTVKRKLLEEEEK | −4.14 | 8.05E−04 |
| | | | | NARLQKELGDVQGHGR | | |
| 1804. | SPECC1 | SPECC1111 | 660 | MAQQELRTVKRKLLEEEEKNARLQK | −2.63 | 1.44E−02 |
| | | | | ELGDVQGHGRVVTSRA | | |
| 1805. | SPECC1 | SPECC1152 | 906 | MSSSWSDGLAFCALLHTYLPAHIPY | −2.18 | 1.95E−02 |
| | | | | QELNSQEKKRNLLLAF | | |
| 1806. | SPECC1 | SPECC1153 | 912 | MGLAFCALLHTYLPAHIPYQELNSQ | −2.12 | 3.27E−02 |
| | | | | EKKRNLLLAFEAAESV | | |
| 1807. | SPECC1 | SPECC1155 | 924 | MPAHIPYQELNSQEKKRNLLLAFEA | −3.29 | 4.21E−03 |
| | | | | AESVGIKPSLELSEML | | |
| 1808. | SPECC1 | SPECC1157 | 936 | MEKKRNLLLAFEAAESVGIKPSLEL | −1.83 | 3.92E−02 |
| | | | | SEMLYTDRPDWQSVMQ | | |
| 1809. | SPOP | SPOP_4 | 18 | MAESWCYTQIKVVKFSYMWTINNFS | −2.41 | 3.25E−02 |
| | | | | FCREEMGEVIKSSTFS | | |
| 1810. | SPOP | SPOP_5 | 24 | MTQIKVVKFSYMWTINNFSFCREEM | −2.32 | 3.84E−02 |
| | | | | GEVIKSSTFSSGANDK | | |
| 1811. | SPTA1 | SPTA1_125 | 744 | MILTDLAAYFEEIGHPDSKDIRARQ | −1.79 | 7.91E−02 |
| | | | | ESLVCRFEALKEPLAT | | |
| 1812. | SPTA1 | SPTA1_126 | 750 | MAYFEEIGHPDSKDIRARQESLVCR | −1.78 | 8.10E−02 |
| | | | | FEALKEPLATRKKKLL | | |
| 1813. | SPTA1 | SPTA1_206 | 1230 | MDLVPLGDKVTILGETAERLSESHP | −2.23 | 4.55E−02 |
| | | | | DATEDLQRQKMELNEA | | |
| 1814. | SPTA1 | SPTA1_208 | 1242 | MGETAERLSESHPDATEDLQRQKME | −2.76 | 1.85E−02 |
| | | | | LNEAWEDLQGRTKDRK | | |
| 1815. | SPTA1 | SPTA1_273 | 1632 | MKDQARDLASAGNLLKKHQLLEREM | −2.28 | 4.32E−02 |
| | | | | LAREDALKDLNTLAED | | |
| 1816. | SPTA1 | SPTA1_276 | 1650 | MLLEREMLAREDALKDLNTLAEDLL | −3.58 | 2.47E−03 |
| | | | | SSGTFNVDQIVKKKDN | | |
| 1817. | SPTA1 | SPTA1_277 | 1656 | MLAREDALKDLNTLAEDLLSSGTFN | −3.63 | 2.26E−03 |
| | | | | VDQIVKKKDNVNKRFL | | |
| 1818. | SPTA1 | SPTA1_308 | 1842 | MVRGDCGDTLAATQSLLMKHEALEN | −4.16 | 7.72E−04 |
| | | | | DFAVHETRVQNVCAQG | | |
| 1819. | SPTA1 | SPTA1_309 | 1848 | MDTLAATQSLLMKHEALENDFAVHE | −4.21 | 6.84E−04 |
| | | | | TRVQNVCAQGEDILNK | | |
| 1820. | SPTAN1 | SPTAN1_131 | 780 | MADSLRLQQLFRDVEDEETWIREKE | −1.78 | 8.96E−02 |
| | | | | PIAASTNRGKDLIGVQ | | |
| 1821. | SPTAN1 | SPTAN1133 | 792 | MVEDEETWIREKEPIAASTNRGKDL | −2.19 | 4.58E−02 |
| | | | | IGVQNLLKKHQALQAE | | |
| 1822. | SPTAN1 | SPTAN1134 | 798 | MWIREKEPIAASTNRGKDLIGVQNL | −2.84 | 1.43E−02 |
| | | | | LKKHQALQAEIAGHEP | | |
| 1823. | SPTAN1 | SPTAN1136 | 810 | MNRGKDLIGVQNLLKKHQALQAEIA | −3.33 | 3.94E−03 |
| | | | | GHEPRIKAVTQKGNAM | | |
| 1824. | SPTAN1 | SPTAN1145 | 864 | MLHELNQKWEALKAKASQRRQDLED | −4.58 | 2.91E−04 |
| | | | | SLQAQQYFADANEAES | | |
| 1825. | SPTAN1 | SPTAN1146 | 870 | MKWEALKAKASQRRQDLEDSLQAQQ | −3.64 | 3.46E−03 |
| | | | | YFADANEAESWMREKE | | |
| 1826. | SPTAN1 | SPTAN1147 | 876 | MAKASQRRQDLEDSLQAQQYFADAN | −2.12 | 3.05E−02 |
| | | | | EAESWMREKEPIVGST | | |
| 1827. | SPTAN1 | SPTAN1148 | 882 | MRQDLEDSLQAQQYFADANEAESWM | −3.08 | 1.08E−02 |
| | | | | REKEPIVGSTDYGKDE | | |
| 1828. | SPTAN1 | SPTAN1234 | 1398 | MEQFGQQLLAHGHYASPEIKQKLDI | −4.30 | 5.65E−04 |
| | | | | LDQERADLEKAWVQRR | | |
| 1829. | SPTAN1 | SPTAN1236 | 1410 | MYASPEIKQKLDILDQERADLEKAW | −3.31 | 4.05E−03 |
| | | | | VQRRMMLDQCLELQLF | | |
| 1830. | SPTAN1 | SPTAN1285 | 1704 | MADISAHEDRLKDLNSQADSLMTSS | −3.04 | 6.45E−03 |
| | | | | AFDTSQVKDKRDTING | | |
| 1831. | SPTAN1 | SPTAN1286 | 1710 | MEDRLKDLNSQADSLMTSSAFDTSQ | −3.12 | 5.68E−03 |
| | | | | VKDKRDTINGRFQKIK | | |
| 1832. | SPTAN1 | SPTAN1288 | 1722 | MSLMTSSAFDTSQVKDKRDTINGRF | −3.45 | 3.15E−03 |
| | | | | QKIKSMAASRRAKLNE | | |
| 1833. | SPTAN1 | SPTAN1305 | 1824 | MDTGKKLSDDNTIGKEEIQQRLAQF | −3.07 | 6.17E−03 |
| | | | | VEHWKELKQLAAARGQ | | |
| 1834. | SPTAN1 | SPTAN1373 | 2232 | MCMVEESGTLESQLEATKRKHQEIR | −2.16 | 2.48E−02 |
| | | | | AMRSQLKKIEDLGAAM | | |
| 1835. | SPTAN1 | SPTAN1_374 | 2238 | MGTLESQLEATKRKHQEIRAMRSQL | −2.61 | 2.48E−02 |
| | | | | KKIEDLGAAMEEALIL | | |
| 1836. | SPTAN1 | SPTAN1375 | 2244 | MLEATKRKHQEIRAMRSQLKKIEDL | −1.78 | 8.44E−02 |
| | | | | GAAMEEALILDNKYTE | | |
| 1837. | SRC | SRC_53 | 312 | MEAQVMKKLRHEKLVQLYAVVSEEP | −2.51 | 1.48E−02 |
| | | | | IYIVTEYMSKGSLLDF | | |
| 1838. | SRC | SRC_55 | 324 | MLVQLYAVVSEEPIYIVTEYMSKGS | −2.49 | 2.52E−02 |
| | | | | LLDFLKGETGKYLRLP | | |
| 1839. | SRC | SRC_56 | 330 | MVVSEEPIYIVTEYMSKGSLLDFLK | −3.35 | 4.07E−03 |
| | | | | GETGKYLRLPQLVDMA | | |
| 1840. | STAG2 | STAG2_40 | 234 | MLNLSINMDNTQRQYEAERNKMIGK | −1.78 | 6.67E−02 |
| | | | | RANERLELLLQKRKEL | | |
| 1841. | STAG2 | STAG2_42 | 246 | MQYEAERNKMIGKRANERLELLLQK | −1.86 | 6.10E−02 |
| | | | | RKELQENQDEIENMMN | | |
| 1842. | STAG2 | STAG2_102 | 606 | MKHLDALLRQIRNIVEKHTDTDVLE | −2.95 | 7.44E−03 |
| | | | | ACSKTYHALCNEEFTI | | |
| 1843. | STAG2 | STAG2_103 | 612 | MLRQIRNIVEKHTDTDVLEACSKTY | −2.12 | 5.57E−02 |
| | | | | HALCNEEFTIFNRVDI | | |
| 1844. | STAG2 | STAG2_104 | 618 | MIVEKHTDTDVLEACSKTYHALCNE | −2.52 | 2.89E−02 |
| | | | | EFTIFNRVDISRSQLI | | |
| 1845. | STAG2 | STAG2_145 | 864 | MLAAFCKLIVYTVVEMNTAADIFKQ | −2.01 | 5.12E−02 |
| | | | | YMKYYNDYGDIIKETM | | |
| 1846. | STAG2 | STAG2_146 | 870 | MLIVYTVVEMNTAADIFKQYMKYYN | −1.87 | 6.75E−02 |
| | | | | DYGDIIKETMSKTRQI | | |
| 1847. | STAG2 | STAG2_147 | 876 | MVEMNTAADIFKQYMKYYNDYGDII | −1.75 | 7.63E−02 |
| | | | | KETMSKTRQIDKIQCA | | |
| 1848. | STAG2 | STAG2_180 | 1074 | MSKPSTGKRKVVEGMQLSLTEESSS | −1.93 | 6.35E−02 |
| | | | | SDSMWLSREQTLHTPV | | |
| 1849. | STAG2 | STAG2_182 | 1086 | MGMQLSLTEESSSSDSMWLSREQTL | −2.33 | 1.06E−02 |
| | | | | HTPVMMQTPQLTSTIM | | |
| 1850. | STAG2 | STAG2_193 | 1152 | MLDYNRRGTSLMEDDEEPIVEDVMM | −3.87 | 1.41E−03 |
| | | | | SSEGRIEDLNEGMDFD | | |
| 1851. | STAG2 | STAG2_195 | 1164 | MDDEEPIVEDVMMSSEGRIEDLNEG | −1.85 | 6.34E−02 |
| | | | | MDFDTMDIDLPPSKNR | | |
| 1852. | STYK1 | STYK1_8 | 42 | MVILWLFIREQRTQQQRSGPQGIAP | −4.58 | 2.89E−04 |
| | | | | VPPPRDLSWEAGHGGN | | |
| 1853. | STYK1 | STYK1_9 | 48 | MIREQRTQQQRSGPQGIAPVPPPRD | −2.00 | 4.35E−02 |
| | | | | LSWEAGHGGNVALPLK | | |
| 1854. | SUSD2 | SUSD2_84 | 498 | MRGTGLTAVAVQEGNSDVVEVRLAN | −1.87 | 3.68E−02 |
| | | | | RTGGLEVLLNQEVLSF | | |
| 1855. | SUSD2 | SUSD2_85 | 504 | MAVAVQEGNSDVVEVRLANRTGGLE | −1.80 | 4.08E−02 |
| | | | | VLLNQEVLSFTEQSWM | | |
| 1856. | SUSD2 | SUSD2_87 | 516 | MEVRLANRTGGLEVLLNQEVLSFTE | −3.44 | 3.24E−03 |
| | | | | QSWMDLKGMFLSVAAG | | |
| 1857. | SUZ12 | SUZ12_22 | 126 | MIKRKTFKVDDMLSKVEKMKGEQES | −2.40 | 3.47E−02 |
| | | | | HSLSAHLQLTFTGFFH | | |
| 1858. | SUZ12 | SUZ12_23 | 132 | MKVDDMLSKVEKMKGEQESHSLSAH | −2.12 | 5.15E−02 |
| | | | | LQLTFTGFFHKNDKPS | | |
| 1859. | SUZ12 | SUZ12_24 | 138 | MSKVEKMKGEQESHSLSAHLQLTFT | −2.03 | 6.14E−02 |
| | | | | GFFHKNDKPSPNSENE | | |
| 1860. | SUZ12 | SUZ12_25 | 144 | MKGEQESHSLSAHLQLTFTGFFHKN | −1.83 | 7.60E−02 |
| | | | | DKPSPNSENEQNSVTL | | |
| 1861. | SUZ12 | SUZ12_40 | 234 | MNEFEPSNSHMVKSYSLLFRVTRPG | −2.04 | 6.18E−02 |
| | | | | RREFNGMINGETNENI | | |
| 1862. | SUZ12 | SUZ12_41 | 240 | MNSHMVKSYSLLFRVTRPGRREFNG | −2.19 | 4.92E−02 |
| | | | | MINGETNENIDVNEEL | | |
| 1863. | SUZ12 | SUZ12_42 | 246 | MSYSLLFRVTRPGRREFNGMINGET | −1.83 | 8.56E−02 |
| | | | | NENIDVNEELPARRKR | | |
| 1864. | SYNPO2 | SYNPO283 | 492 | MLMFAKRRERMDQITAQKEEDKVGG | −2.23 | 4.52E−02 |
| | | | | TPSREQDAAQTDGLRT | | |
| 1865. | SYNPO2 | SYNPO288 | 522 | MDAAQTDGLRTTTSYQRKEEESVRT | −2.66 | 1.19E−02 |
| | | | | QSSVSKSYIEVSHGLG | | |
| 1866. | SYNPO2 | SYNPO290 | 534 | MSYQRKEEESVRTQSSVSKSYIEVS | −3.23 | 4.64E−03 |
| | | | | HGLGHVPQQNGFSGAS | | |
| 1867. | SYNPO2 | SYNPO292 | 546 | MQSSVSKSYIEVSHGLGHVPQQNGF | −2.84 | 9.54E−03 |
| | | | | SGASETANIQRMVPMN | | |
| 1868. | SYNPO2 | SYNPO2_134 | 798 | MKGTVVSSIKIAQPSYPPARPASTL | −3.16 | 5.25E−03 |
| | | | | NVAGPFKGPQAAVASQ | | |
| 1869. | SYNPO2 | SYNPO2_136 | 810 | MPSYPPARPASTLNVAGPFKGPQAA | −3.57 | 3.50E−03 |
| | | | | VASQNYTPKPTVSTPT | | |
| 1870. | SYNPO2 | SYNPO2_142 | 846 | MSTPTVNAVQPGAVGPSNELPGMSG | −2.69 | 1.14E−02 |
| | | | | RGAQLFAKRQSRMEKY | | |
| 1871. | SYNPO2 | SYNPO2143 | 852 | MAVQPGAVGPSNELPGMSGRGAQLF | −2.66 | 1.19E−02 |
| | | | | AKRQSRMEKYVVDSDT | | |
| 1872. | TAF1 | TAF1_105 | 624 | MFGALSQPGPHSVQPLLKHIKKKAK | −2.05 | 5.92E−02 |
| | | | | MREQERQASGGGEMFF | | |
| 1873. | TAF1 | TAF1_107 | 636 | MQPLLKHIKKKAKMREQERQASGGG | −2.77 | 1.00E−02 |
| | | | | EMFFMRTPQDLTGKDG | | |
| 1874. | TAF1 | TAF1_108 | 642 | MIKKKAKMREQERQASGGGEMFFMR | −2.64 | 1.22E−02 |
| | | | | TPQDLTGKDGDLILAE | | |
| 1875. | TAF1 | TAF1_276 | 1650 | MDTNTSLSMSRDASVFQDESNMSVL | −2.83 | 1.10E−02 |
| | | | | DIPSATPEKQVTQEGE | | |
| 1876. | TAF1 | TAF1_277 | 1656 | MSMSRDASVFQDESNMSVLDIPSAT | −2.14 | 2.53E−02 |
| | | | | PEKQVTQEGEDGDGDL | | |
| 1877. | TAF15 | TAF15_75 | 444 | MGYGGDRSGGGYGGDRGGGYGGDRG | −2.32 | 2.96E−02 |
| | | | | GGYGGDRGGGYGGDRG | | |
| 1878. | TAF15 | TAF15_76 | 450 | MSGGGYGGDRGGGYGGDRGGGYGGD | −2.24 | 3.17E−02 |
| | | | | RGGGYGGDRGGYGGDR | | |
| 1879. | TAF15 | TAF15_77 | 456 | MGDRGGGYGGDRGGGYGGDRGGGYG | −2.14 | 3.68E−02 |
| | | | | GDRGGYGGDRGGGYGG | | |
| 1880. | TAL1 | TAL1_42 | 246 | MEEEGTQRAKTGKDPVVGAGGGGGG | −2.08 | 2.75E−02 |
| | | | | GGGGAPPDDLLQDVLS | | |
| 1881. | TBC1D3 | TBC1D334 | 198 | MLYLPEEDAFWALVQLLASERHSLQ | −3.63 | 2.27E−03 |
| | | | | GFHSPNGGTVQGLQDQ | | |
| 1882. | TBC1D3 | TBC1D335 | 204 | MDAFWALVQLLASERHSLQGFHSPN | −3.63 | 2.25E−03 |
| | | | | GGTVQGLQDQQEHVVA | | |
| 1883. | TBC1D3 | TBC1D3_37 | 216 | MERHSLQGFHSPNGGTVQGLQDQQE | −1.79 | 5.82E−02 |
| | | | | HVVATSQPKTMGHQDK | | |
| 1884. | TBL1XR1 | TBL1XR118 | 102 | MLAQQQAAAAAAAAAAASQQGSAKN | −1.87 | 6.55E−02 |
| | | | | GENTANGEENGAHTIA | | |
| 1885. | TBL1XR1 | TBL1XR119 | 108 | MAAAAAAAAAASQQGSAKNGENTAN | −1.85 | 6.67E−02 |
| | | | | GEENGAHTIANNHTDM | | |
| 1886. | TBL1XR1 | TBL1XR120 | 114 | MAAAASQQGSAKNGENTANGEENGA | −1.91 | 6.11E−02 |
| | | | | HTIANNHTDMMEVDGD | | |
| 1887. | TBL1XR1 | TBL1XR124 | 138 | MHTIANNHTDMMEVDGDVEIPPNKA | −2.08 | 4.93E−02 |
| | | | | VVLRGHESEVFICAWN | | |
| 1888. | TBL1XR1 | TBL1XR125 | 144 | MHTDMMEVDGDVEIPPNKAVVLRGH | −2.71 | 2.10E−02 |
| | | | | ESEVFICAWNPVSDLL | | |
| 1889. | TBL1XR1 | TBL1XR133 | 192 | MARIWNLSENSTSGSTQLVLRHCIR | −3.80 | 1.61E−03 |
| | | | | EGGQDVPSNKDVTSLD | | |
| 1890. | TBL1XR1 | TBL1XR135 | 204 | MGSTQLVLRHCIREGGQDVPSNKDV | −3.80 | 1.64E−03 |
| | | | | TSLDWNSEGTLLATGS | | |
| 1891. | TBL1XR1 | TBL1XR164 | 378 | MKQDNCVHDLQAHNKEIYTIKWSPT | −2.09 | 2.70E−02 |
| | | | | GPGTNNPNANLMLASA | | |
| 1892. | TBL1XR1 | TBL1XR1_65 | 384 | MHDLQAHNKEIYTIKWSPTGPGTNN | −3.50 | 2.90E−03 |
| | | | | PNANLMLASASFDSTV | | |
| 1893. | TBL1XR1 | TBL1XR166 | 390 | MNKEIYTIKWSPTGPGTNNPNANLM | −2.82 | 1.72E−02 |
| | | | | LASASFDSTVRLWDVD | | |
| 1894. | TBX3 | TBX3_51 | 300 | MKGFRDTGNGRREKRKQLTLQSMRV | −3.84 | 1.52E−03 |
| | | | | FDERHKKENGTSDESS | | |
| 1895. | TBX3 | TBX3_52 | 306 | MGNGRREKRKQLTLQSMRVFDERHK | −3.82 | 1.56E−03 |
| | | | | KENGTSDESSSEQAAF | | |
| 1896. | TCF12 | TCF12_107 | 636 | MPQTKLLILHQAVAVILSLEQQVRE | −2.12 | 3.67E−02 |
| | | | | RNLNPKAACLKRREEE | | |
| 1897. | TCF12 | TCF12_108 | 642 | MILHQAVAVILSLEQQVRERNLNPK | −2.32 | 2.98E−02 |
| | | | | AACLKRREEEKVSAVS | | |
| 1898. | TCF12 | TCF12_112 | 666 | MAACLKRREEEKVSAVSAEPPTTLP | −3.56 | 4.07E−03 |
| | | | | GTHPGLSETTNPMGHM | | |
| 1899. | TCF3 | TCF3_59 | 348 | MAIHVLRSHAVGTAGDMHTLLPGHG | −3.37 | 3.67E−03 |
| | | | | ALASGFTGPMSLGGRH | | |
| 1900. | TCF3 | TCF3_60 | 354 | MSHAVGTAGDMHTLLPGHGALASGF | −3.44 | 3.21E−03 |
| | | | | TGPMSLGGRHAGLVGG | | |
| 1901. | TCF3 | TCF3_63 | 372 | MALASGFTGPMSLGGRHAGLVGGSH | −3.76 | 4.55E−04 |
| | | | | PEDGLAGSTSLMHNHA | | |
| 1902. | TCF7L2 | TCF7L265 | 384 | MQLYPGWSARDNYGKKKKRKRDKQP | −3.19 | 4.97E−03 |
| | | | | GETNEHSECFLNPCLS | | |
| 1903. | TCF7L2 | TCF7L266 | 390 | MSARDNYGKKKKRKRDKQPGETNEH | −2.91 | 8.04E−03 |
| | | | | SECFLNPCLSLPPITG | | |
| 1904. | TCF7L2 | TCF7L267 | 396 | MGKKKKRKRDKQPGETNEHSECFLN | −1.83 | 8.59E−02 |
| | | | | PCLSLPPITGEKKSAF | | |
| 1905. | TCHP | TCHP_57 | 336 | MQLERAREAELQMLLREEAKEMWEK | −5.02 | 8.87E−05 |
| | | | | REAEWARERSARDRLM | | |
| 1906. | TCHP | TCHP_64 | 378 | MVLTGRQQQIQEKIEQNRRAQEESL | −1.95 | 7.12E−02 |
| | | | | KHREQLIRNLEEVREL | | |
| 1907. | TCHP | TCHP_65 | 384 | MQQIQEKIEQNRRAQEESLKHREQL | −2.06 | 5.13E−02 |
| | | | | IRNLEEVRELARREKE | | |
| 1908. | TCHP | TCHP_67 | 396 | MAQEESLKHREQLIRNLEEVRELAR | −2.15 | 3.11E−02 |
| | | | | REKEESEKLKSARKQE | | |
| 1909. | TCP10L | TCP10L29 | 168 | MSLKIERISSWKTPPQENRDKNLSR | −2.65 | 2.14E−02 |
| | | | | RRQDRRATPTGRPTPC | | |
| 1910. | TCP10L | TCP10L30 | 174 | MISSWKTPPQENRDKNLSRRRQDRR | −2.89 | 9.97E−03 |
| | | | | ATPTGRPTPCAERRGG | | |
| 1911. | TELO2 | TELO2_89 | 528 | MERWEAALRALEGLVYRSPTATREV | −1.98 | 6.65E−02 |
| | | | | SVELAKVLLHLEEKTC | | |
| 1912. | TELO2 | TELO2_90 | 534 | MLRALEGLVYRSPTATREVSVELAK | −2.37 | 1.88E−02 |
| | | | | VLLHLEEKTCVVGFAG | | |
| 1913. | TERT | TERT_20 | 114 | MFTTSVRSYLPNTVTDALRGSGAWG | −2.10 | 2.14E−03 |
| | | | | LLLRRVGDDVLVHLLA | | |
| 1914. | TERT | TERT_21 | 120 | MSYLPNTVTDALRGSGAWGLLLRRV | −5.20 | 5.34E−05 |
| | | | | GDDVLVHLLARCALFV | | |
| 1915. | TET2 | TET2_29 | 168 | MHNCSGPENPELQILNEQEGKSANY | −2.08 | 3.85E−02 |
| | | | | HDKNIVLLKNKAVLMP | | |
| 1916. | TET2 | TET2_30 | 174 | MENPELQILNEQEGKSANYHDKNIV | −2.33 | 2.70E−02 |
| | | | | LLKNKAVLMPNGATVS | | |
| 1917. | TET2 | TET2_58 | 342 | MTTKLASGEEFCSGSSSNLQAPGGS | −3.02 | 6.64E−03 |
| | | | | SERYLKQNEMNGAYFK | | |
| 1918. | TET2 | TET2_59 | 348 | MGEEFCSGSSSNLQAPGGSSERYLK | −2.49 | 2.49E−02 |
| | | | | QNEMNGAYFKQSSVFT | | |
| 1919. | THOC1 | THOC1_82 | 486 | MVNNSNYGWRALRLLARRSPHFFQP | −1.88 | 7.88E−02 |
| | | | | TNQQFKSLPEYLENMV | | |
| 1920. | THOC1 | THOC1_83 | 492 | MGWRALRLLARRSPHFFQPTNQQFK | −3.13 | 5.52E−03 |
| | | | | SLPEYLENMVIKLAKE | | |
| 1921. | THOC1 | THOC1_96 | 570 | MGEQIEVFANKLGEQWKILAPYLEM | −2.47 | 1.58E−02 |
| | | | | KDSEIRQIECDSEDMK | | |
| 1922. | THOC1 | THOC1_97 | 576 | MFANKLGEQWKILAPYLEMKDSEIR | −1.87 | 8.02E−02 |
| | | | | QIECDSEDMKMRAKQL | | |
| 1923. | THRAP3 | THRAP3_116 | 690 | MLAHDEMKSPREPGYKAEGKYKDDP | −2.89 | 1.34E−02 |
| | | | | VDLRLDIERRKKHKER | | |
| 1924. | THRAP3 | THRAP3117 | 696 | MKSPREPGYKAEGKYKDDPVDLRLD | −2.60 | 2.54E−02 |
| | | | | IERRKKHKERDLKRGK | | |
| 1925. | TLR4 | TLR4_5 | 24 | MSWEPCVEVVPNITYQCMELNFYKI | −1.89 | 5.81E−02 |
| | | | | PDNLPFSTKNLDLSFN | | |
| 1926. | TLR4 | TLR4_6 | 30 | MEVVPNITYQCMELNFYKIPDNLPF | −1.77 | 6.54E−02 |
| | | | | STKNLDLSFNPLRHLG | | |
| 1927. | TP53 | TP53_81 | 480 | MLPFALSRGSTEQVGLHWCFVVGRR | −1.87 | 4.80E−02 |
| | | | | MGSRTYQLRFXGFYCE | | |
| 1928. | TP53 | TP53_82 | 486 | MRGSTEQVGLHWCFVVGRRMGSRTY | −2.97 | 8.79E−03 |
| | | | | QLRFXGFYCEGCLGDV | | |
| 1929. | TP53 | TP53_83 | 492 | MVGLHWCFVVGRRMGSRTYQLRFXG | −2.52 | 2.90E−02 |
| | | | | FYCEGCLGDVRNVLAV | | |
| 1930. | TP53BP2 | TP53BP2_21 | 120 | MEQKRLSNGKLVEEIEQMNNLFQQK | −2.12 | 2.62E−02 |
| | | | | QRELVLAVSKVEELTR | | |
| 1931. | TP53BP2 | TP53BP222 | 126 | MNGKLVEEIEQMNNLFQQKQRELVL | −1.91 | 3.50E−02 |
| | | | | AVSKVEELTRQLEMLK | | |
| 1932. | TP53BP2 | TP53BP223 | 132 | MEIEQMNNLFQQKQRELVLAVSKVE | −2.17 | 2.43E−02 |
| | | | | ELTRQLEMLKNGRIDS | | |
| 1933. | TP53BP2 | TP53BP285 | 504 | MFTPQPSKDTLLPPFRKPQTVAASS | −2.94 | 1.35E−02 |
| | | | | IYSMYTQQQAPGKNFQ | | |
| 1934. | TP53BP2 | TP53BP289 | 528 | MIYSMYTQQQAPGKNFQQAVQSALT | −2.99 | 7.03E−03 |
| | | | | KTHTRGPHFSSVYGKP | | |
| 1935. | TP53BP2 | TP53BP2_90 | 534 | MQQQAPGKNFQQAVQSALTKTHTRG | −2.62 | 2.42E−02 |
| | | | | PHFSSVYGKPVIAAAQ | | |
| 1936. | TP53BP2 | TP53BP291 | 540 | MKNFQQAVQSALTKTHTRGPHFSSV | −2.34 | 3.95E−02 |
| | | | | YGKPVIAAAQNQQQHP | | |
| 1937. | TP53BP2 | TP53BP2_92 | 546 | MVQSALTKTHTRGPHFSSVYGKPVI | −2.90 | 8.16E−03 |
| | | | | AAAQNQQQHPENIYSN | | |
| 1938. | TP53BP2 | TP53BP2161 | 960 | MTQCSQFLYGVQEKMGIMNKGVIYA | −1.95 | 4.68E−02 |
| | | | | LWDYEPQNDDELPMKE | | |
| 1939. | TP53BP2 | TP53BP2162 | 966 | MLYGVQEKMGIMNKGVIYALWDYEP | −3.07 | 1.01E−02 |
| | | | | QNDDELPMKEGDCMTI | | |
| 1940. | TP53BP2 | TP53BP2_163 | 972 | MKMGIMNKGVIYALWDYEPQNDDEL | −3.41 | 5.50E−03 |
| | | | | PMKEGDCMTIIHREDE | | |
| 1941. | TP53BP2 | TP53BP2164 | 978 | MKGVIYALWDYEPQNDDELPMKEGD | −2.25 | 3.16E−02 |
| | | | | CMTIIHREDEDEIEWW | | |
| 1942. | TP53BP2 | TP53BP2_168 | 1002 | MCMTIIHREDEDEIEWWWARLNDKE | −2.51 | 2.91E−02 |
| | | | | GYVPRNLLGLYPRIKP | | |
| 1943. | TPM3 | TPM3_18 | 102 | MAQERLATALQKLEEAEKAADESER | −4.20 | 7.02E−04 |
| | | | | GMKVIENRALKDEEKM | | |
| 1944. | TPM3 | TPM3_19 | 108 | MTALQKLEEAEKAADESERGMKVIE | −3.54 | 4.28E−03 |
| | | | | NRALKDEEKMELQEIQ | | |
| 1945. | TPM3 | TPM3_20 | 114 | MEEAEKAADESERGMKVIENRALKD | −3.79 | 2.37E−03 |
| | | | | EEKMELQEIQLKEAKH | | |
| 1946. | TPR | TPR_92 | 546 | MQQQNQRLLVALRELGETREREEQE | −2.67 | 9.45E−03 |
| | | | | TTSSKITELQLKLESA | | |
| 1947. | TPR | TPR_93 | 552 | MLLVALRELGETREREEQETTSSKI | −2.46 | 2.69E−02 |
| | | | | TELQLKLESALTELEQ | | |
| 1948. | TPR | TPR_94 | 558 | MELGETREREEQETTSSKITELQLK | −1.86 | 8.18E−02 |
| | | | | LESALTELEQLRKSRQ | | |
| 1949. | TPR | TPR_130 | 774 | MAVAEVRAENLKKEKEMLKLSEVRL | −3.34 | 3.84E−03 |
| | | | | SQQRESLLAEQRGQNL | | |
| 1950. | TPR | TPR_135 | 804 | MLLAEQRGQNLLLTNLQTIQGILER | −2.84 | 1.33E−02 |
| | | | | SETETKQRLSSQIEKL | | |
| 1951. | TPR | TPR_136 | 810 | MGQNLLLTNLQTIQGILERSETETK | −2.41 | 2.41E−02 |
| | | | | QRLSSQIEKLEHEISH | | |
| 1952. | TPR | TPR_138 | 822 | MQGILERSETETKQRLSSQIEKLEH | −1.98 | 3.19E−02 |
| | | | | EISHLKKKLENEVEQR | | |
| 1953. | TPR | TPR_139 | 828 | MSETETKQRLSSQIEKLEHEISHLK | −2.37 | 1.84E−02 |
| | | | | KKLENEVEQRHTLTRN | | |
| 1954. | TPR | TPR_140 | 834 | MQRLSSQIEKLEHEISHLKKKLENE | −2.23 | 2.23E−02 |
| | | | | VEQRHTLTRNLDVQLL | | |
| 1955. | TPR | TPR_141 | 840 | MIEKLEHEISHLKKKLENEVEQRHT | −2.43 | 2.75E−02 |
| | | | | LTRNLDVQLLDTKRQL | | |
| 1956. | TPR | TPR_142 | 846 | MEISHLKKKLENEVEQRHTLTRNLD | −2.43 | 1.66E−02 |
| | | | | VQLLDTKRQLDTETNL | | |
| 1957. | TPR | TPR_143 | 852 | MKKLENEVEQRHTLTRNLDVQLLDT | −2.33 | 1.58E−02 |
| | | | | KRQLDTETNLHLNTKE | | |
| 1958. | TPR | TPR_160 | 954 | MLKTSTSNVEQYQAMVTSLEESLNK | −3.03 | 6.54E−03 |
| | | | | EKQVTEEVRKNIEVRL | | |
| 1959. | TPR | TPR_161 | 960 | MNVEQYQAMVTSLEESLNKEKQVTE | −3.12 | 5.66E−03 |
| | | | | EVRKNIEVRLKESAEF | | |
| 1960. | TPR | TPR_163 | 972 | MEESLNKEKQVTEEVRKNIEVRLKE | −3.45 | 3.17E−03 |
| | | | | SAEFQTQLEKKLMEVE | | |
| 1961. | TPR | TPR_164 | 978 | MEKQVTEEVRKNIEVRLKESAEFQT | −2.75 | 1.03E−02 |
| | | | | QLEKKLMEVEKEKQEL | | |
| 1962. | TPR | TPR_165 | 984 | MEVRKNIEVRLKESAEFQTQLEKKL | −2.41 | 2.60E−02 |
| | | | | MEVEKEKQELQDDKRR | | |
| 1963. | TPR | TPR_184 | 1098 | MAKEQVSKMASVRQHLEETTQKAES | −2.45 | 2.96E−02 |
| | | | | QLLECKASWEERERML | | |
| 1964. | TPR | TPR_186 | 1110 | MQHLEETTQKAESQLLECKASWEER | −3.26 | 4.44E−03 |
| | | | | ERMLKDEVSKCVCRCE | | |
| 1965. | TPR | TPR_187 | 1116 | MTQKAESQLLECKASWEERERMLKD | −3.07 | 6.15E−03 |
| | | | | EVSKCVCRCEDLEKQN | | |
| 1966. | TPR | TPR_202 | 1206 | MKEIAETRFEVAQVESLRYRQRVEL | −2.27 | 4.41E−02 |
| | | | | LERELQELQDSLNAER | | |
| 1967. | TPR | TPR_203 | 1212 | MRFEVAQVESLRYRQRVELLERELQ | −1.97 | 6.89E−02 |
| | | | | ELQDSLNAEREKVQVT | | |
| 1968. | TPR | TPR_255 | 1524 | MLSRLRQDLQDRTTQEEQLRQQITE | −2.04 | 6.01E−02 |
| | | | | KEEKTRKAIVAAKSKI | | |
| 1969. | TPR | TPR_256 | 1530 | MDLQDRTTQEEQLRQQITEKEEKTR | −2.07 | 5.70E−02 |
| | | | | KAIVAAKSKIAHLAGV | | |
| 1970. | TPR | TPR_257 | 1536 | MTQEEQLRQQITEKEEKTRKAIVAA | −2.01 | 6.36E−02 |
| | | | | KSKIAHLAGVKDQLTK | | |
| 1971. | TPR | TPR_258 | 1542 | MRQQITEKEEKTRKAIVAAKSKIAH | −1.89 | 7.48E−02 |
| | | | | LAGVKDQLTKENEELK | | |
| 1972. | TPR | TPR_307 | 1836 | MDSTIEASDQVSDDTVEMPLPKKLK | −1.81 | 8.26E−02 |
| | | | | SVTPVGTEEEVMAEES | | |
| 1973. | TPR | TPR_308 | 1842 | MSDQVSDDTVEMPLPKKLKSVTPVG | −2.06 | 4.87E−02 |
| | | | | TEEEVMAEESTDGEVE | | |
| 1974. | TPR | TPR_312 | 1866 | MTEEEVMAEESTDGEVETQVYNQDS | −1.80 | 4.08E−02 |
| | | | | QDSIGEGVTQGDYTPM | | |
| 1975. | TPR | TPR_317 | 1896 | MGVTQGDYTPMEDSEETSQSLQIDL | −2.07 | 4.45E−02 |
| | | | | GPLQSDQQTITSSQDG | | |
| 1976. | TPR | TPR_318 | 1902 | MYTPMEDSEETSQSLQIDLGPLQSD | −3.63 | 3.11E−03 |
| | | | | QQTITSSQDGQGKGDD | | |
| 1977. | TPR | TPR_320 | 1914 | MSLQIDLGPLQSDQQTITSSQDGQG | −3.52 | 4.39E−03 |
| | | | | KGDDVIVIDSDDEEED | | |
| 1978. | TPR | TPR_332 | 1986 | MDSNEGTGSADGNDGYEADDAEGGD | −1.97 | 6.94E−02 |
| | | | | GTDPGTETEESMGGGE | | |
| 1979. | TPR | TPR_334 | 1998 | MDGYEADDAEGGDGTDPGTETEESM | −1.99 | 6.16E−02 |
| | | | | GGGEGNHRAADSQNSG | | |
| 1980. | TPR | TPR_335 | 2004 | MDAEGGDGTDPGTETEESMGGGEGN | −3.10 | 5.85E−03 |
| | | | | HRAADSQNSGEGNTGA | | |
| 1981. | TRIM24 | TRIM2436 | 210 | MEAVGVTSQRPVFCPFHKKEQLKLY | −1.94 | 7.27E−02 |
| | | | | CETCDKLTCRDCQLLE | | |
| 1982. | TRIM24 | TRIM2441 | 240 | MLTCRDCQLLEHKEHRYQFIEEAFQ | −2.58 | 2.58E−02 |
| | | | | NQKVIIDTLITKLMEK | | |
| 1983. | TRIM24 | TRIM2442 | 246 | MQLLEHKEHRYQFIEEAFQNQKVII | −2.55 | 2.70E−02 |
| | | | | DTLITKLMEKTKYIKF | | |
| 1984. | TRIM24 | TRIM2443 | 252 | MEHRYQFIEEAFQNQKVIIDTLITK | −2.56 | 2.67E−02 |
| | | | | LMEKTKYIKFTGNQIQ | | |
| 1985. | TRIM24 | TRIM2444 | 258 | MIEEAFQNQKVIIDTLITKLMEKTK | −2.78 | 1.45E−02 |
| | | | | YIKFTGNQIQNRIIEV | | |
| 1986. | TRIM24 | TRIM24144 | 858 | MNFPSGEWICTFCRDLSKPEVEYDC | −1.97 | 4.01E−02 |
| | | | | DAPSHNSEKKKTEGLV | | |
| 1987. | TRIM24 | TRIM24145 | 864 | MWICTFCRDLSKPEVEYDCDAPSHN | −2.24 | 2.20E−02 |
| | | | | SEKKKTEGLVKLTPID | | |
| 1988. | TRIM24 | TRIM24146 | 870 | MRDLSKPEVEYDCDAPSHNSEKKKT | −2.38 | 1.81E−02 |
| | | | | EGLVKLTPIDKRKCER | | |
| 1989. | TRIM27 | TRIM27_60 | 354 | MNLFPCVLGSPCFIAGRHYWEVEVG | −4.17 | 7.55E−04 |
| | | | | DKAKWTIGVCEDSVCR | | |
| 1990. | TRIM27 | TRIM27_61 | 360 | MLGSPCFIAGRHYWEVEVGDKAKWT | −3.63 | 2.24E−03 |
| | | | | IGVCEDSVCRKGGVTS | | |
| 1991. | TRIM37 | TRIM37_61 | 360 | MREFASDFEVGECWGYNRFFRLDLL | −2.17 | 2.43E−02 |
| | | | | ANEGYLNPQNDTVILR | | |
| 1992. | TRIM37 | TRIM37_63 | 372 | MWGYNRFFRLDLLANEGYLNPQNDT | −2.11 | 2.63E−02 |
| | | | | VILRFQVRSPTFFQKS | | |
| 1993. | TRIM37 | TRIM37_68 | 402 | MVRSPTFFQKSRDQHWYITQLEAAQ | −2.42 | 2.55E−02 |
| | | | | TSYIQQINNLKERLTI | | |
| 1994. | TRIM37 | TRIM37_99 | 588 | MGYVGSSSRISRRTHLCSAATSSLL | −3.49 | 4.47E−03 |
| | | | | DIDPLILIHLLDLKDR | | |
| 1995. | TRIM37 | TRIM37_100 | 594 | MSRISRRTHLCSAATSSLLDIDPLI | −4.00 | 1.07E−03 |
| | | | | LIHLLDLKDRSSIENL | | |
| 1996. | TRIM37 | TRIM37101 | 600 | MTHLCSAATSSLLDIDPLILIHLLD | −3.37 | 6.05E−03 |
| | | | | LKDRSSIENLWGLQPR | | |
| 1997. | TTI1 | TTI1_2 | 6 | MPEEAFGVLRPVCVQLTKTQTVENV | −3.06 | 6.26E−03 |
| | | | | EHLQTRLQAVSDSALQ | | |
| 1998. | TTI1 | TTI1_3 | 12 | MVLRPVCVQLTKTQTVENVEHLQTR | −4.73 | 1.96E−04 |
| | | | | LQAVSDSALQELQQYI | | |
| 1999. | TTI1 | TTI1_26 | 150 | MLGFAVSLLLGLAEQEKSKQIKIAA | −2.62 | 2.37E−02 |
| | | | | LKCLQVLLLQCDCQDH | | |
| 2000. | TTI1 | TTI1_27 | 156 | MLLLGLAEQEKSKQIKIAALKCLQV | −3.67 | 2.09E−03 |
| | | | | LLLQCDCQDHPRSLDE | | |
| 2001. | TXNIP | TXNIP_37 | 216 | MARHTYLANGQTKVLTQKLSSVRGN | −1.96 | 3.27E−02 |
| | | | | HIISGTCASWRGKSLR | | |
| 2002. | TXNIP | TXNIP_38 | 222 | MANGQTKVLTQKLSSVRGNHIISGT | −2.35 | 3.84E−02 |
| | | | | CASWRGKSLRVQKIRP | | |
| 2003. | USP44 | USP44_25 | 144 | MQLYTALWHRRRILMGKIFRTWFEQ | −2.90 | 8.17E−03 |
| | | | | SPIGRKKQEEPFQEKI | | |
| 2004. | USP44 | USP44_26 | 150 | MWHRRRILMGKIFRTWFEQSPIGRK | −2.90 | 8.17E−03 |
| | | | | KQEEPFQEKIVVKREV | | |
| 2005. | USP44 | USP44_44 | 258 | MDSSVKRRPIVTPGVTGLRNLGNTC | −2.75 | 1.96E−02 |
| | | | | YMNSVLQVLSHLLIFR | | |
| 2006. | USP44 | USP44_45 | 264 | MRPIVTPGVTGLRNLGNTCYMNSVL | −3.39 | 3.53E−03 |
| | | | | QVLSHLLIFRQCFLKL | | |
| 2007. | USP44 | USP44_65 | 384 | MLHTLFQVMWSGKWALVSPFAMLHS | −1.98 | 1.89E−02 |
| | | | | VWRLIPAFRGYAQQDA | | |
| 2008. | USP6 | USP6_20 | 114 | MLPPVTAREAKKIRREMTRTSKWME | −3.53 | 2.75E−03 |
| | | | | MLGEWETYKHSSKLID | | |
| 2009. | USP6 | USP6_24 | 138 | MMLGEWETYKHSSKLIDRVYKGIPM | −1.84 | 6.62E−02 |
| | | | | NIRGPVWSVLLNIQEI | | |
| 2010. | USP6 | USP6_29 | 168 | MWSVLLNIQEIKLKNPGRYQIMKER | −3.55 | 2.61E−03 |
| | | | | GKRSSEHIHHIDLDVR | | |
| 2011. | USP6 | USP6_30 | 174 | MIQEIKLKNPGRYQIMKERGKRSSE | −2.40 | 3.46E−02 |
| | | | | HIHHIDLDVRTTLRNH | | |
| 2012. | USP9X | USP9X_20 | 114 | MACQRFFRDGLTISFTKILTDEAVS | −3.63 | 3.53E−03 |
| | | | | GWKFEIHRCIINNTHR | | |
| 2013. | USP9X | USP9X_21 | 120 | MRDGLTISFTKILTDEAVSGWKFEI | −3.76 | 2.48E−03 |
| | | | | HRCIINNTHRLVELCV | | |
| 2014. | USP9X | USP9X_218 | 1302 | MLDALSKEKAWQTFIIDLLLHCHSK | −2.43 | 3.28E−02 |
| | | | | TVRQVAQEQFFLMCTR | | |
| 2015. | USP9X | USP9X_219 | 1308 | MEKAWQTFIIDLLLHCHSKTVRQVA | −1.85 | 3.80E−02 |
| | | | | QEQFFLMCTRCCMGHR | | |
| 2016. | USP9X | USP9X_222 | 1326 | MTVRQVAQEQFFLMCTRCCMGHRPL | −2.96 | 1.26E−02 |
| | | | | LFFITLLFTVLGSTAR | | |
| 2017. | USP9X | USP9X_223 | 1332 | MQEQFFLMCTRCCMGHRPLLFFITL | −3.48 | 3.01E−03 |
| | | | | LFTVLGSTARERAKHS | | |
| 2018. | USP9X | USP9X_238 | 1422 | MEETILEGHLGVTKELLAFQTSEKK | −1.85 | 8.35E−02 |
| | | | | FHIGCEKGGANLIKEL | | |
| 2019. | USP9X | USP9X_239 | 1428 | MGHLGVTKELLAFQTSEKKFHIGCE | −2.58 | 1.33E−02 |
| | | | | KGGANLIKELIDDFIF | | |
| 2020. | USP9X | USP9X_240 | 1434 | MKELLAFQTSEKKFHIGCEKGGANL | −2.89 | 8.24E−03 |
| | | | | IKELIDDFIFPASNVY | | |
| 2021. | USP9X | USP9X_241 | 1440 | MQTSEKKFHIGCEKGGANLIKELID | −2.91 | 8.04E−03 |
| | | | | DFIFPASNVYLQYMRN | | |
| 2022. | USP9X | USP9X_294 | 1758 | MKGDLLEGANAYHCEKCNKKVDTVK | −2.36 | 1.52E−02 |
| | | | | RLLIKKLPPVLAIQLK | | |
| 2023. | USP9X | USP9X_295 | 1764 | MGANAYHCEKCNKKVDTVKRLLIKK | −2.54 | 1.16E−02 |
| | | | | LPPVLAIQLKRFDYDW | | |
| 2024. | USP9X | USP9X_296 | 1770 | MCEKCNKKVDTVKRLLIKKLPPVLA | −2.33 | 1.95E−02 |
| | | | | IQLKRFDYDWERECAI | | |
| 2025. | USP9X | USP9X_312 | 1866 | MGVLVHSGQASGGHYYSYIIQRNGG | −3.22 | 8.24E−03 |
| | | | | DGERNRWYKFDDGDVT | | |
| 2026. | USP9X | USP9X_313 | 1872 | MGQASGGHYYSYIIQRNGGDGERNR | −3.22 | 8.09E−03 |
| | | | | WYKFDDGDVTECKMDD | | |
| 2027. | USP9X | USP9X_314 | 1878 | MHYYSYIIQRNGGDGERNRWYKFDD | −4.04 | 9.85E−04 |
| | | | | GDVTECKMDDDEEMKN | | |
| 2028. | USP9X | USP9X_315 | 1884 | MIQRNGGDGERNRWYKFDDGDVTEC | −1.94 | 4.65E−02 |
| | | | | KMDDDEEMKNQCFGGE | | |
| 2029. | USP9X | USP9X_316 | 1890 | MDGERNRWYKFDDGDVTECKMDDDE | −3.14 | 5.42E−03 |
| | | | | EMKNQCFGGEYMGEVF | | |
| 2030. | VAV1 | VAV1_59 | 348 | MKIDGELKITSVERRSKMDRYAFLL | −3.91 | 1.31E−03 |
| | | | | DKALLICKRRGDSYDL | | |
| 2031. | VAV1 | VAV1_60 | 354 | MKITSVERRSKMDRYAFLLDKALLI | −2.24 | 3.70E−02 |
| | | | | CKRRGDSYDLKDFVNL | | |
| 2032. | VAV1 | VAV1_61 | 360 | MRRSKMDRYAFLLDKALLICKRRGD | −1.94 | 6.88E−02 |
| | | | | SYDLKDFVNLHSFQVR | | |
| 2033. | WDR11 | WDR1159 | 348 | MAIRVTKTVRPFSMVCCPVNENAAA | −2.60 | 1.29E−02 |
| | | | | LVVSDGRVMIWELKSA | | |
| 2034. | WDR11 | WDR1161 | 360 | MMVCCPVNENAAALVVSDGRVMIWE | −1.99 | 4.27E−02 |
| | | | | LKSAVCNRNSRNSSSG | | |
| 2035. | WDR11 | WDR1191 | 540 | MGLVRNELQLVDLPTGRSIAFRGER | −2.31 | 2.00E−02 |
| | | | | GNDESAIEMIKVSHLK | | |
| 2036. | WDR11 | WDR1192 | 546 | MLQLVDLPTGRSIAFRGERGNDESA | −2.74 | 1.05E−02 |
| | | | | IEMIKVSHLKQYLAVV | | |
| 2037. | WDR11 | WDR1193 | 552 | MPTGRSIAFRGERGNDESAIEMIKV | −3.81 | 1.59E−03 |
| | | | | SHLKQYLAVVFRDKPL | | |
| 2038. | WDR11 | WDR11126 | 750 | MWVRKIRFAPGKGNQKLIAMYNDGA | −1.92 | 4.32E−02 |
| | | | | EVWDTKEVQMVSSLRS | | |
| 2039. | WDR11 | WDR11129 | 768 | MMYNDGAEVWDTKEVQMVSSLRSGR | −1.84 | 6.35E−02 |
| | | | | NVTFRILDVDWCTSDK | | |
| 2040. | WDR11 | WDR11190 | 1134 | MGCFFSVAETLHSMRYFDRAALFVE | −3.02 | 1.04E−02 |
| | | | | ACLKYGAFEVTEDTEK | | |
| 2041. | WDR11 | WDR11191 | 1140 | MAETLHSMRYFDRAALFVEACLKYG | −3.37 | 6.00E−03 |
| | | | | AFEVTEDTEKLITAIY | | |
| 2042. | WDR11 | WDR11193 | 1152 | MAALFVEACLKYGAFEVTEDTEKLI | −2.75 | 1.49E−02 |
| | | | | TAIYADYARSLKNLGF | | |
| 2043. | XPO1 | XPO1_96 | 570 | MEFMHETHDGVQDMACDTFIKIAQK | −2.74 | 1.28E−02 |
| | | | | CRRHFVQVQVGEVMPF | | |
| 2044. | XPO1 | XPO1_97 | 576 | MHDGVQDMACDTFIKIAQKCRRHFV | −3.05 | 6.33E−03 |
| | | | | QVQVGEVMPFIDEILN | | |
| 2045. | XPO1 | XPO1_135 | 804 | MAIIVNKLGGHITAEIPQIFDAVFE | −2.11 | 2.64E−02 |
| | | | | CTLNMINKDFEEYPEH | | |
| 2046. | XPO1 | XPO1_136 | 810 | MLGGHITAEIPQIFDAVFECTLNMI | −1.75 | 4.33E−02 |
| | | | | NKDFEEYPEHRTNFFL | | |
| 2047. | XPO1 | XPO1_137 | 816 | MAEIPQIFDAVFECTLNMINKDFEE | −2.40 | 2.18E−02 |
| | | | | YPEHRTNFFLLLQAVN | | |
| 2048. | XPO1 | XPO1_152 | 906 | MEEAAAQSFYQTYFCDILQHIFSVV | −1.97 | 5.48E−02 |
| | | | | TDTSHTAGLTMHASIL | | |
| 2049. | XPO1 | XPO1_153 | 912 | MSFYQTYFCDILQHIFSVVTDTSHT | −2.57 | 2.51E−02 |
| | | | | AGLTMHASILAYMFNL | | |
| 2050. | XPO1 | XPO1_154 | 918 | MFCDILQHIFSVVTDTSHTAGLTMH | −2.49 | 2.95E−02 |
| | | | | ASILAYMFNLVEEGKI | | |
| 2051. | XRN1 | XRN1_10 | 54 | MISDD KIFTDIFHYLEVLFRIIKPRKVFFM | −2.02 | 6.18E−02 |
| | | | | AVDGVAPRAKM | | |
| 2052. | XRN1 | XRN1_11 | 60 | MFTDIFHYLEVLFRIIKPRKVFFMA | −1.97 | 6.71E−02 |
| | | | | VDGVAPRAKMNQQRGR | | |
| 2053. | XRN1 | XRN1_13 | 72 | MRIIKPRKVFFMAVDGVAPRAKMNQ | −2.57 | 1.36E−02 |
| | | | | QRGRRFRSAKEAEDKI | | |
| 2054. | XRN1 | XRN1_24 | 138 | MFMARLHEHLKYFVNMKISTDKSWQ | −2.88 | 1.34E−02 |
| | | | | GVTIYFSGHETPGEGE | | |
| 2055. | XRN1 | XRN1_25 | 144 | MEHLKYFVNMKISTDKSWQGVTIYF | −3.45 | 5.04E−03 |
| | | | | SGHETPGEGEHKIMEF | | |
| 2056. | XRN1 | XRN1_34 | 198 | MRHCLYGLDADLIMLGLTSHEAHFS | −2.57 | 2.63E−02 |
| | | | | LLREEVRFGGKKTQRV | | |
| 2057. | XRN1 | XRN1_35 | 204 | MLDADLIMLGLTSHEAHFSLLREEV | −2.46 | 3.13E−02 |
| | | | | RFGGKKTQRVCAPEET | | |
| 2058. | XRN1 | XRN1_37 | 216 | MHEAHFSLLREEVRFGGKKTQRVCA | −2.55 | 2.72E−02 |
| | | | | PEETTFHLLHLSLMRE | | |
| 2059. | XRN1 | XRN1_99 | 588 | MERKRNQHSECLMCWYDRDTEFIYP | −1.77 | 4.25E−02 |
| | | | | SPWPEKFPAIERCCTR | | |
| 2060. | XRN1 | XRN1_101 | 600 | MCWYDRDTEFIYPSPWPEKFPAIER | −2.23 | 3.73E−02 |
| | | | | CCTRYKIISLDAWRVD | | |
| 2061. | XRN1 | XRN1_102 | 606 | MTEFIYPSPWPEKFPAIERCCTRYK | −2.62 | 1.26E−02 |
| | | | | IISLDAWRVDINKNKI | | |
| 2062. | XRN1 | XRN1_103 | 612 | MSPWPEKFPAIERCCTRYKIISLDA | −2.49 | 1.53E−02 |
| | | | | WRVDINKNKITRIDQK | | |
| 2063. | XRN1 | XRN1_144 | 858 | MVFMLGTPYYGCTGEVQDSGDVITE | −2.77 | 1.89E−02 |
| | | | | GRIRVIFSIPCEPNLD | | |
| 2064. | XRN1 | XRN1_145 | 864 | MPYYGCTGEVQDSGDVITEGRIRVI | −3.53 | 2.76E−03 |
| | | | | FSIPCEPNLDALIQNQ | | |
| 2065. | XRN1 | XRN1_146 | 870 | MGEVQDSGDVITEGRIRVIFSIPCE | −3.37 | 3.67E−03 |
| | | | | PNLDALIQNQHKYSIK | | |
| 2066. | XRN1 | XRN1_147 | 876 | MGDVITEGRIRVIFSIPCEPNLDAL | −3.46 | 3.08E−03 |
| | | | | IQNQHKYSIKYNPGYV | | |
| 2067. | XRN1 | XRN1_165 | 984 | MQLLAEYLERAPELFSYIAKNSQED | −3.13 | 5.55E−03 |
| | | | | VFYEDDIWPGENENGA | | |
| 2068. | XRN1 | XRN1_166 | 990 | MLERAPELFSYIAKNSQEDVFYEDD | −3.47 | 3.04E−03 |
| | | | | IWPGENENGAEKVQEI | | |
| 2069. | XRN1 | XRN1_167 | 996 | MLFSYIAKNSQEDVFYEDDIWPGEN | −2.05 | 6.16E−02 |
| | | | | ENGAEKVQEIITWLKG | | |
| 2070. | XRN1 | XRN1_168 | 1002 | MKNSQEDVFYEDDIWPGENENGAEK | −1.99 | 2.58E−02 |
| | | | | VQEIITWLKGHPVSTL | | |
| 2071. | XRN1 | XRN1_212 | 1266 | MVNQHHKSGFNDNSVKYQQRKHDPH | −2.01 | 6.48E−02 |
| | | | | RKFKEECKSPKAECWS | | |
| 2072. | XRN1 | XRN1_214 | 1278 | MSVKYQQRKHDPHRKFKEECKSPKA | −2.32 | 1.97E−02 |
| | | | | ECWSQKMSNKQPNSGI | | |
| 2073. | XRN1 | XRN1_215 | 1284 | MRKHDPHRKFKEECKSPKAECWSQK | −2.47 | 1.58E−02 |
| | | | | MSNKQPNSGIENFLAS | | |
| 2074. | XRN1 | XRN1_216 | 1290 | MRKFKEECKSPKAECWSQKMSNKQP | −2.00 | 2.56E−02 |
| | | | | NSGIENFLASLNISKE | | |
| 2075. | XRN1 | XRN1_217 | 1296 | MCKSPKAECWSQKMSNKQPNSGIEN | −2.24 | 2.20E−02 |
| | | | | FLASLNISKENEVQSS | | |
| 2076. | YAP1 | YAP1_47 | 276 | MAPVKQPPPLAPQSPQGGVMGGSNS | −1.97 | 4.09E−02 |
| | | | | NQQQQMRLQQLQMEKE | | |
| 2077. | YAP1 | YAP1_48 | 282 | MPPLAPQSPQGGVMGGSNSNQQQQM | −1.85 | 7.38E−02 |
| | | | | RLQQLQMEKERLRLKQ | | |
| 2078. | YES1 | YES1_11 | 60 | MPFGGSSGVTPFGGASSSFSVVPSS | −3.08 | 6.03E−03 |
| | | | | YPAGLTGGVTIFVALY | | |
| 2079. | YES1 | YES1_12 | 66 | MGVTPFGGASSSFSVVPSSYPAGLT | −2.87 | 8.52E−03 |
| | | | | GGVTIFVALYDYEART | | |
| 2080. | YES1 | YES1_14 | 78 | MSVVPSSYPAGLTGGVTIFVALYDY | −2.36 | 3.68E−02 |
| | | | | EARTTEDLSFKKGERF | | |
| 2081. | YES1 | YES1_25 | 144 | MNYVAPADSIQAEEWYFGKMGRKDA | −1.86 | 5.62E−02 |
| | | | | ERLLLNPGNORGIFLV | | |
| 2082. | YES1 | YES1_26 | 150 | MDSIQAEEWYFGKMGRKDAERLLLN | −2.20 | 3.59E−02 |
| | | | | PGNQRGIFLVRESETT | | |
| 2083. | YES1 | YES1_27 | 156 | MEWYFGKMGRKDAERLLLNPGNQRG | −2.50 | 2.55E−02 |
| | | | | IFLVRESETTKGAYSL | | |
| 2084. | YES1 | YES1_75 | 444 | MLYGRFTIKSDVWSFGILQTELVTK | −2.24 | 2.22E−02 |
| | | | | GRVPYPGMVNREVLEQ | | |
| 2085. | YES1 | YES1_77 | 456 | MSFGILQTELVTKGRVPYPGMVNRE | −2.42 | 1.70E−02 |
| | | | | VLEQVERGYRMPCPQG | | |
| 2086. | ZBTB16 | ZBTB1681 | 480 | MQTHTGTDMAVFCLLCGKRFQAQSA | −2.21 | 2.30E−02 |
| | | | | LQQHMEVHAGVRSYIC | | |
| 2087. | ZBTB16 | ZBTB1684 | 498 | MFQAQSALQQHMEVHAGVRSYICSE | −2.34 | 3.79E−02 |
| | | | | CNRTFPSHTALKRHLR | | |
| 2088. | ZBTB16 | ZBTB16_96 | 570 | MEKPYECNGCGKKFSLKHQLETHYR | −2.01 | 4.95E−02 |
| | | | | VHTGEKPFECKLCHQR | | |
| 2089. | ZBTB16 | ZBTB1697 | 576 | MNGCGKKFSLKHQLETHYRVHTGEK | −5.59 | 1.49E−05 |
| | | | | PFECKLCHQRSRDYSA | | |
| 2090. | ZBTB20 | ZBTB2091 | 540 | MSFSLKDYLIKHMVTHTGVRAYQCS | −2.17 | 8.83E−03 |
| | | | | ICNKRFTQKSSLNVHM | | |
| 2091. | ZBTB20 | ZBTB2092 | 546 | MYLIKHMVTHTGVRAYQCSICNKRF | −2.75 | 9.42E−03 |
| | | | | TQKSSLNVHMRLHRGE | | |
| 2092. | ZBTB7C | ZBTB7C_12 | 66 | MTLASQPYVYEIDFVQPEALAAILE | −1.86 | 7.23E−03 |
| | | | | FAYTSTLTITAGNVKH | | |
| 2093. | ZBTB7C | ZBTB7C_13 | 72 | MYVYEIDFVQPEALAAILEFAYTST | −2.81 | 2.75E−03 |
| | | | | LTITAGNVKHILNAAR | | |
| 2094. | ZBTB7C | ZBTB7C_16 | 90 | MFAYTSTLTITAGNVKHILNAARML | −4.10 | 3.12E−04 |
| | | | | EIQCIVNVCLEIMEPG | | |
| 2095. | ZBTB7C | ZBTB7C_19 | 108 | MNAARMLEIQCIVNVCLEIMEPGGD | −3.69 | 2.00E−03 |
| | | | | GGEEDDKEDDDDDEDD | | |
| 2096. | ZFYVE19 | ZFYVE1922 | 126 | MRLAALKDERQGSIPSTQEMEARLA | −4.57 | 2.99E−04 |
| | | | | ALQGRVLPSQTPQPAH | | |
| 2097. | ZFYVE19 | ZFYVE1923 | 132 | MDERQGSIPSTQEMEARLAALQGRV | −2.61 | 1.82E−02 |
| | | | | LPSQTPQPAHHTPDTR | | |
| 2098. | ZFYVE19 | ZFYVE1929 | 168 | MPDTRTQAQQTQDLLTQLAAEVAID | −1.91 | 3.50E−02 |
| | | | | ESWKGGGPAASLQNDL | | |
| 2099. | ZFYVE19 | ZFYVE1932 | 186 | MAEVAIDESWKGGGPAASLQNDLNQ | −2.39 | 2.74E−02 |
| | | | | GGPGSTNSKRQANWSL | | |
| 2100. | ZMYM2 | ZMYM2130 | 774 | MGTLKERVQWRGEMKHFCDQHCLLR | −1.77 | 9.29E−02 |
| | | | | FYCQQNEPNMTTQKGP | | |
| 2101. | ZMYM2 | ZMYM2131 | 780 | MVQWRGEMKHFCDQHCLLRFYCQQN | −1.75 | 7.39E−02 |
| | | | | EPNMTTQKGPENLHYD | | |
| 2102. | ZMYM3 | ZMYM3100 | 594 | MHSLFSGKPEVLDWQDQVFQFCCRD | −3.87 | 6.00E−04 |
| | | | | CCEDFKRLRGVVSQCE | | |
| 2103. | ZMYM3 | ZMYM3101 | 600 | MKPEVLDWQDQVFQFCCRDCCEDFK | −3.36 | 3.74E−03 |
| | | | | RLRGVVSQCEHCRQEK | | |
| 2104. | ZMYM3 | ZMYM3102 | 606 | MWQDQVFQFCCRDCCEDFKRLRGVV | −3.20 | 4.87E−03 |
| | | | | SQCEHCRQEKLLHEKL | | |
| 2105. | ZNF133 | ZNF13384 | 498 | MGRGFSQKSNLVAHQRTHSGERPYV | −5.10 | 7.01E−05 |
| | | | | CRECGRGFSHQAGLIR | | |
| 2106. | ZNF133 | ZNF133_86 | 510 | MHQRTHSGERPYVCRECGRGFSHQA | −4.40 | 4.63E−04 |
| | | | | GLIRHKRKHSREKPYM | | |
| 2107. | ZNF133 | ZNF13390 | 534 | MGLIRHKRKHSREKPYMCRQCGLGF | −4.08 | 9.18E−04 |
| | | | | GNKSALITHKRAHSEE | | |
| 2108. | ZNF133 | ZNF13391 | 540 | MRKHSREKPYMCRQCGLGFGNKSAL | −2.22 | 2.37E−02 |
| | | | | ITHKRAHSEEKPCVCR | | |
| 2109. | ZNF320 | ZNF32012 | 66 | MEVIHTGTLQRQASYHIGAFCSQEI | −2.24 | 3.61E−02 |
| | | | | EKDIHDFVFQWQEDET | | |
| 2110. | ZNF320 | ZNF32014 | 78 | MSYHIGAFCSQEIEKDIHDFVFQWQ | −1.98 | 5.39E−02 |
| | | | | EDETNDHEAPMTEIKK | | |
| 2111. | ZNF320 | ZNF32021 | 120 | MSSTDRYDQRHAGNKPIKGQLESRF | −2.82 | 1.35E−02 |
| | | | | HLHLRRHRRIHTGEKP | | |
| 2112. | ZNF320 | ZNF32022 | 126 | MDQRHAGNKPIKGQLESRFHLHLRR | −2.12 | 5.16E−02 |
| | | | | HRRIHTGEKPYKCEEC | | |
| 2113. | ZNF320 | ZNF32023 | 132 | MNKPIKGQLESRFHLHLRRHRRIHT | −2.39 | 3.60E−02 |
| | | | | GEKPYKCEECEKVFSC | | |
| 2114. | ZNF320 | ZNF32024 | 138 | MQLESRFHLHLRRHRRIHTGEKPYK | −2.98 | 7.13E−03 |
| | | | | CEECEKVFSCKSHLEI | | |
| 2115. | ZNF320 | ZNF32025 | 144 | MHLHLRRHRRIHTGEKPYKCEECEK | −1.92 | 2.84E−02 |
| | | | | VFSCKSHLEIHRIIHT | | |
| 2116. | ZNF320 | ZNF32049 | 288 | MVIHKAVHTAEKPYKCNECGKVFKQ | −1.81 | 8.60E−02 |
| | | | | RATLAGHRRVHTGEKP | | |
| 2117. | ZNF320 | ZNF32050 | 294 | MHTAEKPYKCNECGKVFKQRATLAG | −2.97 | 7.28E−03 |
| | | | | HRRVHTGEKPYRCEEC | | |
| 2118. | ZNF320 | ZNF32055 | 324 | MGEKPYRCEECDKVFSRKSHLERHR | −2.69 | 1.67E−02 |
| | | | | RIHTGEKPYKCKVCDK | | |
| 2119. | ZNF320 | ZNF32056 | 330 | MCEECDKVFSRKSHLERHRRIHTGE | −2.57 | 2.64E−02 |
| | | | | KPYKCKVCDKAFRSDS | | |
| 2120. | ZNF320 | ZNF32057 | 336 | MVFSRKSHLERHRRIHTGEKPYKCK | −3.24 | 4.59E−03 |
| | | | | VCDKAFRSDSRLAEHQ | | |
| 2121. | ZNF320 | ZNF32072 | 426 | MHLERHRRIHTGEKPHKCGDCGKAF | −2.43 | 3.37E−02 |
| | | | | NSPSHLIRHQRIHTGQ | | |
| 2122. | ZNF320 | ZNF32073 | 432 | MRIHTGEKPHKCGDCGKAFNSPSHL | −3.03 | 6.58E−03 |
| | | | | IRHQRIHTGQKSYKCH | | |
| 2123. | ZNF521 | ZNF521163 | 972 | MTLTEHKVTHSKSLDTGNCRICKMP | −2.49 | 1.53E−02 |
| | | | | LQSEEEFLEHCQMHPD | | |
| 2124. | ZNF521 | ZNF521164 | 978 | MVTHSKSLDTGNCRICKMPLQSEEE | −1.86 | 7.65E−02 |
| | | | | FLEHCQMHPDLRNSLT | | |

TABLE 4
Exemplary peptides significantly altering A-to-I RNA editing
A-to-I peptide inhibitors (SEQ ID NOs: 2125-2147),
cut-off < -2 Standard Deviation A-to-I editing < 15% basal;
<27% IFNα. A-to-I peptide activators (SEQ ID NOs: 2148-2210),
cut-off > 2 Standard Deviation A-to-I editing > 37% basal; >47% IFNα.

| | | | | | | % A- | % A- |
|---|---|---|---|---|---|---|---|
| SEQ | | | AA | | Fitness | to-I | to-I |
| ID | Driver | Peptide | start | | Score | editing | editing |
| NO: | ID | ID | position | Amino Acid Sequence | IFNα | (Basal) | (IFNα) |
| 2125. | ADAD1 | ADAD1_25 | 96 | MPVSALHQFAQMQRVQLDLKET | 0.99 | 14.03 | 25.66 |
| | | | | VTTGNVMGPYFAFCAVVDG | | | |
| 2126. | ADAR | ADAR_156 | 620 | MECMHKLGNSCEFRLLSKEGPA | 0.35 | 13.60 | 20.83 |
| | | | | HEPKFQYCVAVGAQTFPSV | | | |
| 2127. | ADAR | ADAR_193 | 768 | MFPAVCAHSKKQGKQEAADAAL | 0.57 | 11.47 | 22.77 |
| | | | | RVLIGENEKAERMGFTEVT | | | |
| 2128. | CCDC124 | CCDC124_ | 92 | MSKVTRAQIEDTLRRDHQLREA | -1.31 | 12.50 | 13.63 |
| | | 24 | | PDTAEKAKSHLEVPLEENV | | | |
| 2129. | CCDC124 | CCDC124_ | 104 | MRRDHQLREAPDTAEKAKSHLE | 1.71 | 12.18 | 24.35 |
| | | 27 | | VPLEENVNRRVLEEGSVEA | | | |
| 2130. | CDKN2AIP | CDKN2AIP_ | 184 | MSESGNSARSSGISSQNSSTSD | -1.31 | 14.82 | 26.81 |
| | | 47 | | GDRSVSSQSSSSVSSQVTT | | | |
| 2131. | EP300 | EP300_306 | 1220 | MQPQTTINKEQFSKRKNDTLDP | -0.25 | 12.58 | 20.81 |
| | | | | ELFVECTECGRKMHQICVL | | | |
| 2132. | EP300 | EP300_310 | 1236 | MDTLDPELFVECTECGRKMHQI | -1.83 | 14.29 | 26.86 |
| | | | | CVLHHEIIWPAGFVCDGCL | | | |
| 2133. | FSCN1 | FSCN1_76 | 300 | MDTKKCAFRTHTGKYWTLTATG | 0.04 | 12.90 | 25.01 |
| | | | | GVQSTASSKNASCYFDIEW | | | |
| 2134. | FSCN1 | FSCN1_83 | 328 | MSKNASCYFDIEWRDRRITLRA | -0.67 | 14.68 | 25.99 |
| | | | | SNGKFVTSKKNGQLAASVE | | | |
| 2135. | FSCN1 | FSCN1_104 | 412 | MVFQLEFNDGAYNIKDSTGKYW | 0.43 | 13.62 | 26.41 |
| | | | | TVGSDSAVTSSGDTPVDFF | | | |
| 2136. | HTRA4 | HTRA4_36 | 140 | MKVPAVPVQWGNCGDTGTRSAG | 0.18 | 12.55 | 25.56 |
| | | | | PLRRNYNFIAAVVEKVAPS | | | |
| 2137. | SMC1A | SMC1A_48 | 188 | MKKNIAAERKEAKQEKEEADRY | 1.61 | 11.19 | 26.12 |
| | | | | QRLKDEVVRAQVQLQLFKL | | | |
| 2138. | SMC1A | SMC1A_73 | 288 | MSELNQKRPQYIKAKENTSHKI | -0.76 | 11.31 | 23.54 |
| | | | | KKLEAAKKSLQNAQKHYKK | | | |
| 2139. | SMC3 | SMC3_228 | 908 | MKEHMDAINHDTKELEKMTNRQ | -3.54 | 13.27 | 23.22 |
| | | | | GMLLKKKEECMKKIRELGS | | | |
| 2140. | SON | SON_53 | 208 | MSQHRKKEADSVYGEWVPVEKN | 1.03 | 14.38 | 25.27 |
| | | | | GEENKDDDNVFSSNLPSEP | | | |
| 2141. | SOX2 | SOX2_17 | 64 | MKMHNSEISKRLGAEWKLLSET | 1.12 | 12.15 | 23.50 |
| | | | | EKRPFIDEAKRLRALHMKE | | | |
| 2142. | STAU1 | STAU1_42 | 164 | MLPERLEVNGRESEEENLNKSE | 0.46 | 11.00 | 18.40 |
| | | | | ISQVFEIALKRNLPVNFEV | | | |
| 2143. | STAU2 | STAU2_44 | 172 | MNKSEISLVFEIALKRNMPVSF | 0.07 | 6.78 | 23.54 |
| | | | | EVIKESGPPHMKSFVTRVS | | | |
| 2144. | STRBP | STRBP_120 | 476 | METVSSNSSNNTGNSTTETSST | 0.07 | 10.91 | 22.73 |
| | | | | LEVRTQGPILTASGKNPVM | | | |
| 2145 | UPF1 | UPF1_147 | 584 | MGELSSADEKRYRALKRTAERE | -0.63 | 13.30 | 24.68 |
| | | | | LLMNADVICCTCVGAGDPR | | | |
| 2146. | WWP2 | WWP2_185 | 736 | MLRYFDEKELELMLCGMQEIDM | -0.08 | 13.42 | 25.97 |
| | | | | SDWQKSTIYRHYTKNSKQI | | | |
| 2147. | XPO1 | XPO1_167 | 664 | MWDSIIQQATKNVDILKDPETV | 1.42 | 11.93 | 25.96 |
| | | | | KQLGSILKTNVRACKAVGH | | | |
| 2148. | ADAD2 | ADAD2_6 | 20 | MASLQISPQPRPWRPLPAQAQS | 3.12 | 40.89 | 48.57 |
| | | | | AWGPAPAPATYRAEGGWPQ | | | |
| 2149. | ADAD2 | ADAD2_70 | 276 | MGQQLHDCHGLVIARRALLRFL | 0.13 | 37.53 | 49.55 |
| | | | | FRQLLLATQGGPKGKEQSV | | | |
| 2150. | ADAD2 | ADAD2_71 | 280 | MHDCHGLVIARRALLRFLFRQL | 0.34 | 37.15 | 55.50 |
| | | | | LLATQGGPKGKEQSVLAPQ | | | |
| 2151. | ADAD2 | ADAD2_72 | 284 | MGLVIARRALLRFLFRQLLLAT | 0.62 | 52.83 | 58.83 |
| | | | | QGGPKGKEQSVLAPQPGPG | | | |
| 2152. | ADAD2 | ADAD2_99 | 392 | MCLSASDKLARWAVLGLGGALL | 1.22 | 39.54 | 47.44 |
| | | | | AHLVSPLYSTSLILADSCH | | | |
| 2153. | ADAD2 | ADAD2_129 | 512 | MGPPSRLCKASFLRAFHQAARA | -0.55 | 39.07 | 47.45 |
| | | | | VGKPYLLALKTYEAAKAGP | | | |
| 2154. | ADAR | ADAR_32 | 124 | MCLSSHFQELSIYQDQEQRILK | -0.47 | 38.66 | 48.60 |
| | | | | FLEELGEGKATTAHDLSGK | | | |
| 2155. | ADAR | ADAR_97 | 384 | MRNAEFLTCNIPTSNASNNMVT | -1.27 | 40.12 | 51.11 |
| | | | | TEKVENGQEPVIKLENRQE | | | |
| 2156. | ADAR | ADAR_231 | 920 | MRFLYSELMKYNSQTAKDSIFE | 0.14 | 56.37 | 65.21 |
| | | | | PAKGGEKLQIKKTVSFHLY | | | |
| 2157. | ADAR | ADAR_236 | 940 | MEPAKGGEKLQIKKTVSFHLYI | -1.07 | 39.57 | 55.48 |
| | | | | STAPCGDGALFDKSCSDRA | | | |
| 2158. | ADARB1 | ADARB1_ | 500 | MTWDGVLQGERLLTMSCSDKIA | -0.18 | 51.13 | 67.20 |
| | | 126 | | RWNVVGIQGSLLSIFVEPI | | | |
| 2159. | ADARB1 | ADARB1_ | 504 | MVLQGERLLTMSCSDKIARWNV | 0.26 | 60.17 | 65.17 |
| | | 127 | | VGIQGSLLSIFVEPIYFSS | | | |
| 2160 | ADARB2 | ADARB2_ | 196 | MQAHLAMGGGPGPGTDFTSDQA | 0.65 | 40.11 | 51.37 |
| | | 50 | | DFPDTLFQEFEPPAPRPGL | | | |
| 2161. | BRIX1 | BRIX1_17 | 64 | MFSSRGINFRTRHLMQDLRMLM | 1.21 | 38.66 | 47.02 |
| | | | | PHSKADTKMDRKDKLFVIN | | | |
| 2162. | DGCR8 | DGCR8_7 | 24 | MRPFQALPREQSPPPPLQTSSG | 0.13 | 40.23 | 54.04 |
| | | | | AEVMDVGSGGDGQSELPAE | | | |
| 2163. | DHX9 | DHX9_28 | 108 | MMGGPLPPHLALKAENNSEVGA | 1.46 | 49.65 | 54.35 |
| | | | | SGYGVPGPTWDRGANLKDY | | | |
| 2164. | DHX9 | DHX9_307 | 1224 | MGFRGNSGGDYRGPSGGYRGSG | 1.50 | 40.04 | 47.59 |
| | | | | GFQRGGGRGAYGTGYFGQG | | | |
| 2165. | DICER1 | DICER1_31 | 120 | MVGEYSNLEVNASWTKERWNQE | 0.94 | 37.36 | 48.57 |
| | | | | FTKHQVLIMTCYVALNVLK | | | |
| 2166. | DICER1 | DICER1_98 | 388 | MPYERQQFESVEWYNNRNQDNY | -1.79 | 37.74 | 52.37 |
| | | | | VSWSDSEDDDEDEEIEEKE | | | |
| 2167. | DVL2 | DVL2_126 | 500 | MCYYVFGDLSGGCESYLVNLSL | 0.54 | 43.60 | 54.28 |
| | | | | NDNDGSSGASDQDTLAPLP | | | |
| 2168. | HDAC5 | HDAC5_73 | 288 | MVISTFKKRAVEITGAGPGASS | 0.74 | 49.71 | 63.84 |
| | | | | VCNSAPGSGPSSPNSSHST | | | |
| 2169. | HDAC5 | HDAC5_74 | 292 | MFKKRAVEITGAGPGASSVCNS | 0.29 | 38.94 | 55.72 |
| | | | | APGSGPSSPNSSHSTIAEN | | | |
| 2170. | HDAC5 | HDAC5_75 | 296 | MAVEITGAGPGASSVCNSAPGS | 0.76 | 40.79 | 55.71 |
| | | | | GPSSPNSSHSTIAENGFTG | | | |
| 2171. | HDAC5 | HDAC5_161 | 640 | MVYQAPLSLATVPHQALGRTQS | -0.12 | 43.88 | 48.17 |
| | | | | SPAAPGGMKSPPDQPVKHL | | | |
| 2172. | ILF2 | ILF2_50 | 196 | MVLQSALAAIRHARWFEENASQ | 0.42 | 43.18 | 47.06 |
| | | | | STVKVLIRLLKDLRIRFPG | | | |
| 2173. | ILF2 | ILF2_51 | 200 | MALAAIRHARWFEENASQSTVK | 0.27 | 43.99 | 48.52 |
| | | | | VLIRLLKDLRIRFPGFEPL | | | |
| 2174. | ILF3 | ILF3_62 | 244 | MPLRGWPLELLCEKSIGTANRP | -0.73 | 39.78 | 55.34 |
| | | | | MGAGEALRRVLECLASGIV | | | |
| 2175. | NIPBL | NIPBL_167 | 664 | MESTIVEPKQNENRLSDTKPND | 1.11 | 39.35 | 52.90 |
| | | | | NKQNNGRSETTKSRPETPK | | | |
| 2176. | NIPBL | NIPBL_285 | 1136 | MGRRSSGGGRYRNRSPSDSDME | 1.59 | 42.29 | 47.59 |
| | | | | DYSPPPSLSEVARKMKKKE | | | |
| 2177. | NIPBL | NIPBL_452 | 1804 | MSILARLDMQRGVHGRLMDNST | 2.31 | 38.35 | 50.76 |
| | | | | SVREAAVELLGRFVLCRPQ | | | |
| 2178. | PPP2CB | PPP2CB_43 | 168 | MGLSPSIDTLDHIRALDRLQEV | -0.38 | 46.04 | 62.33 |
| | | | | PHEGPMCDLLWSDPDDRGG | | | |
| 2179. | PRKRA | PRKRA_9 | 32 | MKTPIQVLHEYGMKTKNIPVYE | -0.21 | 43.02 | 48.95 |
| | | | | CERSDVQIHVPTFTFRVTV | | | |
| 2180. | RAD21 | RAD21_124 | 492 | MQVEQMEIPPVELPPEEPPNIC | 1.20 | 39.15 | 48.70 |
| | | | | QLIPELELLPEKEKEKEKE | | | |
| 2181. | SIRT6 | SIRT6_22 | 34 | MTFESARPTQTHMALVQLERVG | -0.38 | 49.44 | 58.51 |
| | | | | LLRFLVSQNVDGLHVRSGF | | | |
| 2182. | SIRT6 | SIRT6_23 | 88 | MARPTQTHMALVQLERVGLLRF | 0.14 | 57.96 | 62.85 |
| | | | | LVSQNVDGLHVRSGFPRDK | | | |
| 2183. | SIRT6 | SIRT6_26 | 100 | MLERVGLLRFLVSQNVDGLHVR | -1.23 | 49.42 | 54.71 |
| | | | | SGFPRDKLAELHGNMFVEE | | | |
| 2184. | SMC1A | SMC1A_186 | 740 | MLANFGPRINDIKRIIQSRERE | 0.69 | 55.39 | 56.93 |
| | | | | MKDLKEKMNQVEDEVFEEF | | | |
| 2185. | SMC3 | SMC3_31 | 120 | MVMNLLESAGFSRSNPYYIVKQ | 1.72 | 41.67 | 54.66 |
| | | | | GKINQMATAPDSQRLKLLR | | | |
| 2186. | SMC3 | SMC3_243 | 968 | MLEQCNTELKKYSHVNKKALDQ | -0.61 | 42.74 | 47.01 |
| | | | | FVNFSEQKEKLIKRQEELD | | | |
| 2187. | SNRPB | SNRPB_24 | 92 | MARVPLAGAAGGPGIGRAAGRG | -0.23 | 40.00 | 54.10 |
| | | | | IPAGVPMPQAPAGLAGPVR | | | |
| 2188. | SNRPB | SNRPB_27 | 104 | MGIGRAAGRGIPAGVPMPQAPA | -0.15 | 41.09 | 51.33 |
| | | | | GLAGPVRGVGGPSQQVMTP | | | |
| 2189. | SNRPN | SNRPN_22 | 84 | MPPPKDTGIARVPLAGAAGGPG | 0.37 | 51.65 | 63.73 |
| | | | | VGRAAGRGVPAGVPIPQAP | | | |
| 2190. | SNRPN | SNRPN_23 | 88 | MDTGIARVPLAGAAGGPGVGRA | 0.20 | 44.79 | 56.44 |
| | | | | AGRGVPAGVPIPQAPAGLA | | | |
| 2191. | SNRPN | SNRPN_25 | 96 | MLAGAAGGPGVGRAAGRGVPAG | 0.43 | 44.62 | 61.35 |
| | | | | VPIPQAPAGLAGPVRGVGG | | | |
| 2192. | SOX2 | SOX2_58 | 228 | MQQGTPGMALGSMGSVVKSEAS | -0.36 | 41.35 | 53.74 |
| | | | | SSPPVVTSSSHSRAPCQAG | | | |
| 2193. | STAG1 | STAG1_49 | 192 | MIIYDEYMMDTVISLLTGLSDS | 0.55 | 44.91 | 48.18 |
| | | | | QVRAFRHTSTLAAMKLMTA | | | |
| 2194. | STAG1 | STAG1_51 | 200 | MDTVISLLTGLSDSQVRAFRHT | -0.15 | 74.10 | 80.32 |
| | | | | STLAAMKLMTALVNVALNL | | | |
| 2195. | STAG1 | STAG1_52 | 204 | MSLLTGLSDSQVRAFRHTSTLA | -0.26 | 46.34 | 58.66 |
| | | | | AMKLMTALVNVALNLSIHQ | | | |
| 2196. | STAG1 | STAG1_54 | 212 | MSQVRAFRHTSTLAAMKLMTAL | 0.28 | 43.52 | 54.81 |
| | | | | VNVALNLSIHQDNTQRQYE | | | |
| 2197. | STAG2 | STAG2_21 | 80 | MNMMLFEVVKMGKSAMQSVVDD | 0.65 | 43.58 | 58.92 |
| | | | | WIESYKHDRDIALLDLINF | | | |
| 2198. | UBE2I | UBE2I_32 | 124 | MIQDPAQAEAYTIYWLVAALAP | -0.02 | 44.43 | 47.26 |
| | | | | LVAAPPRPSQGRLAGREWS | | | |
| 2199. | UPF1 | UPF1_52 | 204 | MVVLLCRQPCASQSSLKDINWD | -0.80 | 50.56 | 47.63 |
| | | | | SSQWQPLIQDRCFLSWLVK | | | |
| 2200. | WAPAL | WAPAL_208 | 828 | MKECVDHLSRDEDEEKLVASLW | 1.55 | 50.48 | 50.33 |
| | | | | GAERCLRVLESVTVHNPEN | | | |
| 2201. | ZFR | ZFR_18 | 68 | MAPVAAHTVTAAYAPAAATVAV | 1.04 | 37.25 | 49.47 |
| | | | | ARPAPVAVAAAATAAAYGG | | | |
| 2202. | ZFR | ZFR_66 | 260 | MSGTSYSGYEAAVYSAASSYYQ | 2.04 | 40.81 | 49.01 |
| | | | | QQQQQQKQAAAAAAAAAAT | | | |
| 2203. | ZFR | ZFR_67 | 264 | MYSGYEAAVYSAASSYYQQQQQ | 2.43 | 41.43 | 47.21 |
| | | | | QQKQAAAAAAAAAATAAWT | | | |
| 2204. | ZFR | ZFR_148 | 588 | MCSFNDPNAKEMHLKGRRHRLQ | -1.00 | 37.77 | 48.58 |
| | | | | YKKKVNPDLQVEVKPSIRA | | | |
| 2205. | ZFR2 | ZFR2_183 | 728 | MQVTISVTSPLMREDPSTDPGV | -0.70 | 40.13 | 54.85 |
| | | | | EEPQADAGDVLSPKKCLES | | | |
| 2206. | ZFR2 | ZFR2_205 | 816 | MVEKAVSSAAGPLGPGDAVRRV | 0.12 | 45.00 | 54.50 |
| | | | | LECVATGTLLTDGPGLQDP | | | |
| 2207. | ZFR2 | ZFR2_206 | 820 | MVSSAAGPLGPGDAVRRVLECV | 0.81 | 44.05 | 55.48 |
| | | | | ATGTLLTDGPGLQDPCERD | | | |
| 2208. | ZFR2 | ZFR2_207 | 824 | MAGPLGPGDAVRRVLECVATGT | 1.51 | 45.30 | 51.31 |
| | | | | LLTDGPGLQDPCERDQTDA | | | |
| 2209. | ZFR2 | ZFR2_208 | 828 | MGPGDAVRRVLECVATGTLLTD | 0.67 | 37.66 | 51.37 |
| | | | | GPGLQDPCERDQTDALEPM | | | |
| 2210. | ZFR2 | ZFR2_209 | 832 | MAVRRVLECVATGTLLTDGPGL | 0.22 | 39.66 | 50.31 |
| | | | | QDPCERDQTDALEPMTLQE | | | |

TABLE 5
Exemplary peptides significantly altering IFNα cell fitness effect.
Synthetic lethal peptides (SEQ ID NOs: 2211-2243) or protector peptides
(SEQ ID NOs: 2244-2327) upon IFNα treatment.

| | | | AA | | Fitness | |
|---|---|---|---|---|---|---|
| SEQ ID | Driver | Peptide | start | | Score | |
| NO: | ID | ID | position | Amino Acid Sequence | IFNα | FDR |
| 2211. | ADAR | ADAR_37 | 144 | MKFLEELGEGKATTAHDLSGKLGTPK | -2.18 | 1.19E-02 |
| | | | | KEINRVLYSLAKKGK | | |
| 2212. | CDKN2AIP | CDKN2AIP_ | 80 | MAWANHVFLGCRYPQKVMDKILSMAE | -2.59 | 2.62E-02 |
| | | 21 | | GIKVTDAPTYTTRDE | | |
| 2213. | DICER1 | DICER1_20 | 76 | MLTKELSYQIRGDFSRNGKRTVFLVN | -2.89 | 5.66E-03 |
| | | | | SANQVAQQVSAVRTH | | |
| 2214. | DICER1 | DICER1_411 | 1640 | MCLKIPPRCMFDHPDADKTLNHLISG | -2.46 | 2.95E-03 |
| | | | | FENFEKKINYRFKNK | | |
| 2215. | DICER1 | DICER1_442 | 1764 | MLFHVIDDFVQFQLEKNEMQGMDSEL | -2.09 | 8.42E-06 |
| | | | | RRSEEDEEKEEDIEV | | |
| 2216. | EBNA1BP2 | EBNA1BP2_ | 156 | MKQAAMERSEKAKQLRALRKYGKKVQ | -2.51 | 4.26E-02 |
| | | 40 | | TEVLQKRQQEKAHMM | | |
| 2217. | EP300 | EP300_156 | 620 | MESANNRAEYYHLLAEKIYKIQKELE | -4.04 | 4.51E-02 |
| | | | | EKRRTRLQKQNMLPN | | |
| 2218. | EP300 | EP300_246 | 980 | MKMEVDQPEPADTQPEDISESKVEDC | -3.11 | 4.49E-04 |
| | | | | KMESTETEERSTELK | | |
| 2219. | HDAC5 | HDAC5_138 | 548 | MPEETEEELTEQQEVLLGEGALTMPR | -15.49 | 4.67E-04 |
| | | | | EGSTESESTQEDLEE | | |
| 2220. | HDAC5 | HDAC5_198 | 788 | MDTVWNEMHSSSAVRMAVGCLLELAF | -2.40 | 3.83E-07 |
| | | | | KVAAGELKNGFAIIR | | |
| 2221. | HDAC5 | HDAC5_226 | 900 | MFPGSGAPEEVGGGPGVGYNVNVAWT | -11.88 | 5.23E-03 |
| | | | | GGVDPPIGDVEYLTA | | |
| 2222. | HDLBP | HDLBP_17 | 64 | MGNKIRPIKASVITQVFHVPLEERKY | -2.05 | 3.32E-04 |
| | | | | KDMNQFGEGEQAKIC | | |
| 2223. | ILF2 | ILF2_88 | 348 | MVIVTPSEKAYEKPPEKKEGEEEEEN | -12.75 | 3.34E-03 |
| | | | | TEEPPQGEEEESMET | | |
| 2224. | NIPBL | NIPBL_145 | 576 | MPVSVLQEDIVGSLKSTPENHPETPK | -2.10 | 2.57E-03 |
| | | | | KKSDPELSKSEMKQS | | |
| 2225. | NIPBL | NIPBL_287 | 1144 | MRYRNRSPSDSDMEDYSPPPSLSEVA | -2.61 | 7.53E-05 |
| | | | | RKMKKKEKQKKRKAY | | |
| 2226. | PIN1 | PIN1_13 | 48 | MQGEPARVRCSHLLVKHSQSRRPSSW | -3.17 | 1.71E-03 |
| | | | | RQEKITRTKEEALEL | | |
| 2227. | RAD21 | RAD21_67 | 264 | MDEDDNVSMGGPDSPDSVDPVEPMPT | -2.61 | 4.63E-03 |
| | | | | MTDQTTLVPNEEEAF | | |
| 2228. | SMC1A | SMC1A_107 | 424 | MLREIEENQKRIEKLEEYITTSKQSL | -5.6 | 1.17E-05 |
| | | | | EEQKKLEGELTEEVE | | |
| 2229. | SMC1A | SMC1A_113 | 448 | MLEEQKKLEGELTEEVEMAKRRIDEI | -2.30 | 1.10E-04 |
| | | | | NKELNQVMEQLGDAR | | |
| 2230. | SMC1A | SMC1A_139 | 552 | MGRDCIQYIKEQRGEPETFLPLDYLE | -2.43 | 1.84E-04 |
| | | | | VKPTDEKLRELKGAK | | |
| 2231. | SMC3 | SMC3_161 | 640 | MQLARAFTMDCITLEGDQVSHRGALT | -2.23 | 1.15E-05 |
| | | | | GGYYDTRKSRLELQK | | |
| 2232. | SMC3 | SMC3_197 | 784 | MLLSQLSLEDQKRVDALNDEIRQLQQ | -2.89 | 2.71E-03 |
| | | | | ENRQLLNERIKLEGI | | |
| 2233. | SMC3 | SMC3_228 | 908 | MKEHMDAINHDTKELEKMTNRQGMLL | -3.54 | 3.08E-03 |
| | | | | KKKEECMKKIRELGS | | |
| 2234. | SMC3 | SMC3_247 | 984 | MKALDQFVNFSEQKEKLIKRQEELDR | -4.15 | 2.68E-03 |
| | | | | GYKSIMELMNVLELR | | |
| 2235. | STAG1 | STAG1_9 | 32 | MGKRKRGRPGRPPSTNKKPRKSPGEK | -2.27 | 5.18E-05 |
| | | | | SRIEAGIRGAGRGRA | | |
| 2236. | STAG1 | STAG1_208 | 828 | MLLSFVMDHVFIDQDEENQSMEGDEE | -2.77 | 4.44E-03 |
| | | | | DEANKIEALHKRRNL | | |
| 2237. | STAG1 | STAG1_287 | 1144 | MDFLHNRGLMEEDAEPIFEDVMMSSR | -4.96 | 8.30E-04 |
| | | | | SQLEDMNEEFEDTMV | | |
| 2238. | STAU1 | STAU1_120 | 476 | MSSGHVPHGPLTRPSEQLDYLSRVQG | -2.32 | 2.45E-03 |
| | | | | FQVEYKDFPKNNKNE | | |
| 2239. | STRBP | STRBP_106 | 420 | MSVDVDGTTYEASGPSKKTAKLHVAV | -2.29 | 5.46E-03 |
| | | | | KVLQAMGYPTGFDAD | | |
| 2240. | UPF1 | UPF1_220 | 876 | MKALSKQPLWNHLLNYYKEQKVLVEG | -2.21 | 3.28E-06 |
| | | | | PLNNLRESLMQFSKP | | |
| 2241. | XPO1 | XPO1_255 | 1016 | MDFLVQIKEFAGEDTSDLFLEEREIA | -2.94 | 3.83E-03 |
| | | | | LRQADEEKHKRQMSV | | |
| 2242. | ZFR | ZFR_161 | 640 | MQKEEYWRRREEEERWRMEMRRYEED | -4.76 | 2.93E-03 |
| | | | | MYWRRMEEEQHHWDD | | |
| 2243. | ZFR2 | ZFR2_67 | 264 | MPRQLQLHYCDICKISCAGPQTYREH | -2.66 | 4.08E-03 |
| | | | | LGGQKHRKKEAAQKT | | |
| 2244. | ADAD2 | ADAD2_6 | 20 | MASLQISPQPRPWRPLPAQAQSAWGP | 3.12 | 7.54E-25 |
| | | | | APAPATYRAEGGWPQ | | |
| 2245. | ADAD2 | ADAD2_7 | 24 | MISPQPRPWRPLPAQAQSAWGPAPAP | 2.77 | 1.98E-21 |
| | | | | ATYRAEGGWPQVSVL | | |
| 2246. | ADAD2 | ADAD2_9 | 32 | MRPLPAQAQSAWGPAPAPATYRAEGG | 2.81 | 5.16E-03 |
| | | | | WPQVSVLRDSGPGAG | | |
| 2247. | ADAD2 | ADAD2_73 | 288 | MARRALLRFLFRQLLLATQGGPKGKE | 2.55 | 5.03E-03 |
| | | | | QSVLAPQPGPGPPFT | | |
| 2248. | ADARB2 | ADARB2_15 | 56 | MEDDDTLSTSSAEVKENRNVGNLAAR | 3.58 | 2.73E-36 |
| | | | | PPPSGDRARGGAPGA | | |
| 2249. | ADARB2 | ADARB2_16 | 60 | MTLSTSSAEVKENRNVGNLAARPPPS | 2.27 | 6.60E-13 |
| | | | | GDRARGGAPGAKRKR | | |
| 2250. | ADARB2 | ADARB2_17 | 64 | MSSAEVKENRNVGNLAARPPPSGDRA | 3.36 | 2.01E-15 |
| | | | | RGGAPGAKRKRPLEE | | |
| 2251. | ADARB2 | ADARB2_55 | 216 | MADFPDTLFQEFEPPAPRPGLAGGRP | 2.18 | 5.42E-04 |
| | | | | GDAALLSAAYGRRRL | | |
| 2252. | ADARB2 | ADARB2_56 | 220 | MDTLFQEFEPPAPRPGLAGGRPGDAA | 3.07 | 1.56E-35 |
| | | | | LLSAAYGRRRLLCRA | | |
| 2253. | ADARB2 | ADARB2_57 | 224 | MQEFEPPAPRPGLAGGRPGDAALLSA | 4.39 | 0.00E+00 |
| | | | | AYGRRRLLCRALDLV | | |
| 2254. | ADARB2 | ADARB2_58 | 228 | MPPAPRPGLAGGRPGDAALLSAAYGR | 7.49 | 0.00E+00 |
| | | | | RRLLCRALDLVGPTP | | |
| 2255. | ADARB2 | ADARB2_59 | 232 | MRPGLAGGRPGDAALLSAAYGRRRLL | 3.78 | 4.61E-61 |
| | | | | CRALDLVGPTPATPA | | |
| 2256. | ADARB2 | ADARB2_61 | 240 | MPGDAALLSAAYGRRRLLCRALDLVG | 5.45 | 6.96E-61 |
| | | | | PTPATPAAPGERNPV | | |
| 2257. | ADARB2 | ADARB2_62 | 244 | MALLSAAYGRRRLLCRALDLVGPTPA | 4.15 | 2.30E-25 |
| | | | | TPAAPGERNPVVLLN | | |
| 2258. | ADARB2 | ADARB2_63 | 248 | MAAYGRRRLLCRALDLVGPTPATPAA | 5.79 | 3.73E-35 |
| | | | | PGERNPVVLLNRLRA | | |
| 2259. | ADARB2 | ADARB2_64 | 252 | MRRRLLCRALDLVGPTPATPAAPGER | 2.07 | 6.50E-05 |
| | | | | NPVVLLNRLRAGLRY | | |
| 2260. | ADARB2 | ADARB2_65 | 256 | MLCRALDLVGPTPATPAAPGERNPVV | 2.11 | 3.55E-12 |
| | | | | LLNRLRAGLRYVCLA | | |
| 2261. | BTF3 | BTF3_21 | 80 | MASLAANTFTITGHAETKQLTEMLPS | 2.29 | 2.74E-03 |
| | | | | ILNQLGADSLTSLRR | | |
| 2262. | CDKN2AIP | CDKN2AIP_ | 144 | MVIEGKNSSAVEQDHAKTSAKTERAS | 2.86 | 1.16E-04 |
| | | 37 | | AQQENSSTCIGSAIK | | |
| 2263. | DGCR8 | DGCR8_160 | 636 | MRVGKQLASQKILQLLHPHVKNWGSL | 3.24 | 6.98E-08 |
| | | | | LRMYGRESSKMVKQE | | |
| 2264. | DHX9 | DHX9_289 | 1152 | MTRYGDGPRPPKMARYDNGSGYRRGG | 2.16 | 3.40E-05 |
| | | | | SSYSGGGYGGGYSSG | | |
| 2265. | DICER1 | DICER1_65 | 256 | MCGPFTDRSGLYERLLMELEEALNFI | 2.62 | 1.87E-03 |
| | | | | NDCNISVHSKERDST | | |
| 2266. | DICER1 | DICER1_116 | 460 | MIKEAGKQDPELAYISSNFITGHGIG | 2.40 | 2.08E-04 |
| | | | | KNQPRNKQMEAEFRK | | |
| 2267. | EP300 | EP300_304 | 1212 | MVSLGDDPSQPQTTINKEQFSKRKND | 2.38 | 4.08E-02 |
| | | | | TLDPELFVECTECGR | | |
| 2268. | EP300 | EP300_375 | 1496 | MSAKELPYFEGDFWPNVLEESIKELE | 2.84 | 1.56E-03 |
| | | | | QEEEERKREENTSNE | | |
| 2269. | EP300 | EP300_491 | 1960 | MPMAPMGMNPPPMTRGPSGHLEPGMG | 3.66 | 2.86E-04 |
| | | | | PTGMQQQPPWSQGGL | | |
| 2270. | ILF2 | ILF2_20 | 76 | MLVTKINNVIDNLIVAPGTFEVQIEE | 2.11 | 9.54E-03 |
| | | | | VRQVGSYKKGTMTTG | | |
| 2271. | ILF3 | ILF3_110 | 436 | MEVDGNSFEASGPSKKTAKLHVAVKV | 2.58 | 3.85E-04 |
| | | | | LQDMGLPTGAEGRDS | | |
| 2272. | ILF3 | ILF3_113 | 448 | MSKKTAKLHVAVKVLQDMGLPTGAEG | 2.71 | 4.56E-03 |
| | | | | RDSSKGEDSAEETEA | | |
| 2273. | NIPBL | NIPBL_147 | 584 | MIVGSLKSTPENHPETPKKKSDPELS | 3.53 | 1.82E-05 |
| | | | | KSEMKQSESRLAESK | | |
| 2274. | NIPBL | NIPBL_265 | 1056 | MAEIESTMPLCERVKMNKRKRSTVNE | 3.18 | 6.37E-05 |
| | | | | KPKYAEISSDEDNDS | | |
| 2275. | NIPBL | NIPBL_452 | 1804 | MSILARLDMQRGVHGRLMDNSTSVRE | 2.31 | 1.30E-03 |
| | | | | AAVELLGRFVLCRPQ | | |
| 2276. | NIPBL | NIPBL_457 | 1824 | MTSVREAAVELLGRFVLCRPQLAEQY | 2.54 | 3.66E-02 |
| | | | | YDMLIERILDTGISV | | |
| 2277. | NIPBL | NIPBL_657 | 2624 | MMEHLDPDEEEEEGEVSASTNARNKA | 2.33 | 4.47E-04 |
| | | | | ITSLLGGGSPKNNTA | | |
| 2278. | PDS5A | PDS5A_101 | 400 | MGFVRERTLDKRWRVRKEAMMGLAQL | 2.40 | 4.21E-02 |
| | | | | YKKYCLHGEAGKEAA | | |
| 2279. | RAD21 | RAD21_35 | 136 | MQSRVEEITMREEVGNISILQENDFG | 2.53 | 4.77E-12 |
| | | | | DFGMDDREIMREGSA | | |
| 2280. | RAD21 | RAD21_55 | 216 | MDDNFGEGNDGGILDDKLISNNDGGI | 2.84 | 1.33E-02 |
| | | | | FDDPPALSEAGVMLP | | |
| 2281. | RAD21 | RAD21_96 | 380 | MWNNRLLKLFTRCLTPLVPEDLRKRR | 2.84 | 5.91E-04 |
| | | | | KGGEADNLDEFLKEF | | |
| 2282. | SMC1A | SMC1A_83 | 328 | MRKGDMDELEKEMLSVEKARQEFEER | 3.47 | 1.27E-10 |
| | | | | MEEESQSQGRDLTLE | | |
| 2283. | SMC1A | SMC1A_88 | 348 | MEFEERMEEESQSQGRDLTLEENQVK | 2.76 | 9.72E-03 |
| | | | | KYHRLKEEASKRAAT | | |
| 2284. | SMC1A | SMC1A_184 | 732 | MEKSKLESELANFGPRINDIKRIIQS | 2.80 | 4.68E-03 |
| | | | | REREMKDLKEKMNQV | | |
| 2285. | SMC1A | SMC1A_193 | 768 | MMNQVEDEVFEEFCREIGVRNIREFE | 3.73 | 1.41E-12 |
| | | | | EEKVKRQNEIAKKRL | | |
| 2286. | SMC1A | SMC1A_203 | 808 | MEFENQKTRLGIQLDFEKNQLKEDQD | 2.28 | 1.95E-02 |
| | | | | KVHMWEQTVKKDENE | | |
| 2287. | SMC1A | SMC1A_205 | 816 | MLGIQLDFEKNQLKEDQDKVHMWEQT | 3.38 | 7.50E-06 |
| | | | | VKKDENEIEKLKKEE | | |
| 2288. | SMC3 | SMC3_8 | 28 | MVIVGRNGSGKSNLFYAIQFVLSDEF | 2.21 | 6.50E-03 |
| | | | | SHLRPEQRLALLHEG | | |
| 2289. | SMC3 | SMC3_140 | 556 | MRLFYHIVDSDEVSTKILMEFNKMNL | 2.65 | 3.76E-04 |
| | | | | PGEVTFLPLNKLDVR | | |
| 2290. | SMC3 | SMC3_163 | 648 | MDCITLEGDQVSHRGALTGGYYDTRK | 2.32 | 4.86E-03 |
| | | | | SRLELQKDVRKAEEE | | |
| 2291. | SMC3 | SMC3_168 | 668 | MYDTRKSRLELQKDVRKAEEELGELE | 2.07 | 6.33E-04 |
| | | | | AKLNENLRRNIERIN | | |
| 2292. | SMC3 | SMC3_191 | 760 | MQSLEASLHAMESTRESLKAELGTDL | 3.05 | 1.72E-12 |
| | | | | LSQLSLEDQKRVDAL | | |
| 2293. | SMC3 | SMC3_205 | 816 | MERIKLEGIITRVETYLNENLRKRLD | 2.66 | 1.99E-04 |
| | | | | QVEQELNELRETEGG | | |
| 2294. | SMURF2 | SMURF2_94 | 372 | MVQKLKILRQELSQQQPQAGHCRIEV | 2.33 | 3.89E-05 |
| | | | | SREEIFEESYRQVMK | | |
| 2295. | SMURF2 | SMURF2_135 | 536 | MLENDITGVLDHTFCVEHNAYGEIIQ | 2.04 | 1.23E-02 |
| | | | | HELKPNGKSIPVNEE | | |
| 2296. | SOX2 | SOX2_2 | 4 | MMETELKPPGPQQTSGGGGGNSTAAA | 3.30 | 2.54E-04 |
| | | | | AGGNQKNSPDRVKRP | | |
| 2297. | SOX2 | SOX2_30 | 116 | MKTLMKKDKYTLPGGLLAPGGNSMAS | 2.64 | 2.10E-07 |
| | | | | GVGVGAGLGAGVNQR | | |
| 2298. | SOX2 | SOX2_31 | 120 | MKKDKYTLPGGLLAPGGNSMASGVGV | 4.05 | 8.22E-57 |
| | | | | GAGLGAGVNQRMDSY | | |
| 2299. | SPRTN | SPRTN_9 | 32 | MDASWELVDPTPDLQALFVQFNDQFF | 3.58 | 2.81E-05 |
| | | | | WGQLEAVEVKWSVRM | | |
| 2300. | STAG1 | STAG1_120 | 476 | MLHEHAAYLVDSLWESSQELLKDWEC | 3.15 | 1.45E-03 |
| | | | | MTELLLEEPVQGEEA | | |
| 2301. | STAG1 | STAG1_207 | 824 | MLQSELLSFVMDHVFIDQDEENQSME | 2.04 | 4.87E-02 |
| | | | | GDEEDEANKIEALHK | | |
| 2302. | STAG1 | STAG1_256 | 1020 | MTVHSYLEKFLTEQMMERREDVWLPL | 2.31 | 2.29E-02 |
| | | | | ISYRNSLVTGGEDDR | | |
| 2303. | STAG2 | STAG2_11 | 40 | MKTCKKGKKGPAEKGKGGNGGGKPPS | 3.48 | 8.05E-05 |
| | | | | GPNRMNGHHQQNGVE | | |
| 2304. | STAG2 | STAG2_57 | 224 | MKLMTALVNVALNLSINMDNTQRQYE | 2.21 | 3.50E-02 |
| | | | | AERNKMIGKRANERL | | |
| 2305. | STAG2 | STAG2_126 | 500 | MLLLEEPLSGEEALTDRQESALIEIM | 2.36 | 5.21E-06 |
| | | | | LCTIRQAAECHPPVG | | |
| 2306. | STAG2 | STAG2_184 | 732 | MALQCTHYVILWQLAKITESSSTKED | 3.20 | 1.05E-03 |
| | | | | LLRLKKQMRVFCQIC | | |
| 2307. | STAG2 | STAG2_257 | 1024 | MFMTFQMSLRREDVWLPLMSYRNSLL | 3.08 | 4.69E-21 |
| | | | | AGGDDDTMSVISGIS | | |
| 2308. | STAU1 | STAU1_86 | 340 | MKRNAAENMLEILGFKVPQAQPTKPA | 2.84 | 6.89E-04 |
| | | | | LKSEEKTPIKKPGDG | | |
| 2309. | STAU1 | STAU1_90 | 356 | MPQAQPTKPALKSEEKTPIKKPGDGR | 4.14 | 2.02E-12 |
| | | | | KVTFFEPGSGDENGT | | |
| 2310. | STRBP | STRBP_110 | 436 | MKTAKLHVAVKVLQAMGYPTGFDADI | 2.20 | 1.66E-04 |
| | | | | ECMSSDEKSDNESKN | | |
| 2311. | WAPAL | WAPAL_58 | 228 | MKGSVRTGLFEWDNDFEDIRSEDCIL | 2.72 | 6.89E-04 |
| | | | | SLDSDPLLEMKDDDF | | |
| 2312. | WAPAL | WAPAL_110 | 436 | MGGDEGGSGSSNYKIKYFGFDDLSES | 4.46 | 1.78E-23 |
| | | | | EDDEDDDCQVERKTS | | |
| 2313. | WAPAL | WAPAL_258 | 1028 | MQVHAVQALVQLFLERERAAQLAESK | 2.65 | 4.21E-02 |
| | | | | TDELIKDAPTTQHDK | | |
| 2314. | WWP2 | WWP2_164 | 652 | MNSIVWIKENNLEECGLELYFIQDME | 2.39 | 1.27E-06 |
| | | | | ILGKVTTHELKEGGE | | |
| 2315. | WWP2 | WWP2_191 | 760 | MQKSTIYRHYTKNSKQIQWFWQVVKE | 3.88 | 1.78E-13 |
| | | | | MDNEKRIRLLQFVTG | | |
| 2316. | XPO1 | XPO1_30 | 116 | MPTCVEKEKVYIGKLNMILVQILKQE | 3.24 | 1.04E-03 |
| | | | | WPKHWPTFISDIVGA | | |
| 2317. | XPO1 | XPO1_159 | 632 | MFYEAVGYMIGAQTDQTVQEHLIEKY | 2.65 | 2.11E-02 |
| | | | | MLLPNQVWDSIIQQA | | |
| 2318. | XPO1 | XPO1_203 | 808 | MNKLGGHITAEIPQIFDAVFECTLNM | 2.14 | 1.44E-09 |
| | | | | INKDFEEYPEHRTNF | | |
| 2319. | ZFR | ZFR_66 | 260 | MSGTSYSGYEAAVYSAASSYYQQQQQ | 2.04 | 5.30E-06 |
| | | | | QQKQAAAAAAAAAAT | | |
| 2320. | ZFR | ZFR_67 | 264 | MYSGYEAAVYSAASSYYQQQQQQQKQ | 2.43 | 4.80E-06 |
| | | | | AAAAAAAAAATAAWT | | |
| 2321. | ZFR | ZFR_204 | 812 | MSEKPSKTLLSRIAENLPKQLAVISP | 2.57 | 1.79E-03 |
| | | | | EKYDIKCAVSEAAII | | |
| 2322. | ZFR | ZFR_234 | 932 | MCQRVPTWSDFPSWAMELLVEKAISS | 2.87 | 1.43E-02 |
| | | | | ASSPQSPGDALRRVF | | |
| 2323. | ZFR2 | ZFR2_131 | 520 | MRMRKQRHLAEERLEQLRRWHAERRR | 4.23 | 4.14E-25 |
| | | | | LEEEPPQDVPPHAPP | | |
| 2324. | ZFR2 | ZFR2_132 | 524 | MQRHLAEERLEQLRRWHAERRRLEEE | 5.31 | 7.26E-138 |
| | | | | PPQDVPPHAPPDWAQ | | |
| 2325. | ZFR2 | ZFR2_133 | 528 | MAEERLEQLRRWHAERRRLEEEPPQD | 4.20 | 1.44E-68 |
| | | | | VPPHAPPDWAQPLLM | | |
| 2326. | ZFR2 | ZFR2_202 | 804 | MWGALPAWAMELLVEKAVSSAAGPLG | 2.82 | 2.89E-04 |
| | | | | PGDAVRRVLECVATG | | |
| 2327. | ZFR2 | ZFR2_225 | 896 | MGMDLLPPRHRLGARFRKRQRGPGEG | 3.45 | 2.13E-05 |
| | | | | EEGAGEKKRGRRGGE | | |

TABLE 6
Exemplary Peptide Transduction Domain (PTD)
or Cell-Penetrating Peptide (CPP) sequences

| SEQ ID | | |
|---|---|---|
| NO: | Peptide ID | Amino Acid Sequence |
| 2328. | TAT | RKKRRQRRR |
| 2329. | Penetratin | RQIKIWFQNRRMKWKK |
| 2330. | Buforin II | TRSSRAGLQFPVGRVHRLLRK |
| 2331. | Transportan | GWTLNSAGYLLGKINKALAALAKKIL |
| 2332. | MAP (model | KLALKLALKALKAALKLA |
| | amphipathicpeptide) | |
| 2333. | K-FGF | AAVALLPAVLLALLAP |
| 2334. | Ku70 | VPMLK-PMLKE |
| 2335. | Prion | MANLGYWLLALFVTMWTDVGLCKKRPKP |
| 2336. | pVEC | LLIILRRRIRKQAHAHSK |
| 2337. | Pep-1 | KETWWETWWTEWSQPKKKRKV |
| 2338. | SynB1 | RGGRLSYSRRRFSTSTGR |
| 2339. | Pep-7 (phage display) | SDLWEMMMVSLACQY |
| 2340. | HN-1 (phage display) | TSPLNIHNGQKL |
| 2341. | PTD sequence 1 | KKRPKPG |
| 2342. | PTD sequence 2 | RKKRRQRRR |
| 2343. | TAT CPP (2) | GRKKRRQRRRPPQ |
| 2344. | Maurocalcine | GDCLPHLKLCKENKDCCSKKCKRRGTNIEKRCR |
| 2345. | p Vec CPP (2) | LLILRRRIRKQAHAHSK |
| 2346. | Transportan CPP (2) | GWTLNSAGYLLGKINLKALAALAKKIL |
| 2347. | CADY | GLWRALWRLLRSLWRLLWRA |
| 2348. | p28 | LSTAADMQGWTDGMASGLDKDYLKPDD |

TABLE 7
Exemplary Linkers for Peptide
Transduction Domain (PTD) or Cell-
Penetrating Peptide (CPP) sequences

| SEQ ID | Peptide | |
|---|---|---|
| NO: | ID | Amino Acid Sequence |
| 2349. | Linker 1 | GGGGS |
| 2350. | Linker 2 | GKSSGSGSESKS |
| 2351. | Linker 3 | GSTSGSGKSSEGKG |
| 2352. | Linker 4 | GSTSGSGKSSEGSGSTKG |
| 2353. | Linker 5 | GSTSGSGKPGSGEGSTKG |
| 2354. | Linker 6 | EGKSSGSGSESKEF |

### P EMBODIMENTS

P Embodiment 1. A pharmaceutical composition in unit dose form comprising: (a) a peptide or salt thereof; and (b) at least one of a pharmaceutically acceptable: excipient, diluent, or carrier, wherein the peptide or salt thereof has at least about 80% sequence identity to a polypeptide of any one of the sequences provided herein; wherein the peptide or salt thereof: (i) modulates an expression level of a target protein implicated in a disease or condition, as measured by an at least partial increase or an at least partial decrease of a level of the target protein in an in vitro assay in a cell treated with the peptide or salt thereof as determined by a Western blot relative to a level of the target protein in an otherwise comparable cell not treated with the peptide or salt thereof; (ii) produces an at least partial increase or an at least partial decrease of an activity of the target protein, as measured by a level of the activity of the target protein in a cell treated with the peptide or salt thereof relative to a level of activity of the target protein in an otherwise comparable cell not treated with the peptide or salt thereof as determined by an in vitro assay; (iii) produces an at least partial increase or an at least partial decrease of an activity of a protein downstream of the target protein in a cellular pathway in a cell treated with the peptide or salt thereof relative to a level of activity of the protein downstream of the target protein in a cellular pathway in an otherwise comparable cell not treated with the peptide or salt thereof as determined by an in vitro assay; (iv) kills a cancer cell in an in vitro assay; or (v) any combination thereof.

P Embodiment 2. The pharmaceutical composition of P embodiment 1, wherein the peptide or salt thereof modulates the expression level of the target protein implicated in the disease or condition, as measured by the at least partial increase or the at least partial decrease of the level of the target protein in the in vitro assay in the cell treated with the peptide or salt thereof as determined by the Western blot relative to the level of the target protein in the otherwise comparable cell not treated with the peptide or salt thereof.

P Embodiment 3. The pharmaceutical composition of P embodiment 1, wherein the peptide or salt thereof produces the at least partial increase or the at least partial decrease of the activity of the target protein, as measured by the level of the activity of the target protein in the cell treated with the peptide or salt thereof relative to the level of activity of the target protein in the otherwise comparable cell not treated with the peptide or salt thereof as determined by the in vitro assay.

P Embodiment 4. The pharmaceutical composition of P embodiment 1, wherein the peptide or salt thereof produces the at least partial increase or the at least partial decrease of the activity of the protein downstream of the target protein in a cellular pathway in the cell treated with the peptide or salt thereof relative to the level of the activity of the protein downstream of the target protein in the cellular pathway in the otherwise comparable cell not treated with the peptide or salt thereof as determined by the in vitro assay.

P Embodiment 5. The pharmaceutical composition of P embodiment 1, wherein the peptide or salt thereof kills the cancer cell in the in vitro assay.

P Embodiment 6. The pharmaceutical composition of P embodiment 1, wherein the peptide or salt thereof modulates the target protein by at least partially inhibiting a protein to protein interaction.

P Embodiment 7. The pharmaceutical composition of P embodiment 1, wherein the peptide or salt thereof at least partially reduces a protein to nucleic acid interaction.

P Embodiment 8. The pharmaceutical composition of P embodiment 1, wherein the peptide comprises independently Gly, or an amino acid comprising a C₁-C₁₀, alkyl, a C₁-C₁₀ alkenyl, a C₁-C₁₀ alkynyl, a cycloalkyl, or an alkylcycloalkyl side chain.

P Embodiment 9. The pharmaceutical composition of P embodiment 1, wherein the peptide comprises an amino acid comprising an aromatic side chain.

P Embodiment 10. The pharmaceutical composition of P embodiment 1, wherein the peptide comprises an amino acid comprising a side chain that is at least partially protonated at a pH of about 7.3.

P Embodiment 11. The pharmaceutical composition of P embodiment 1, wherein the peptide comprises an amino acid comprising an amide containing side chain.

P Embodiment 12. The pharmaceutical composition of P embodiment 1, wherein the peptide comprises an amino acid comprising an alcohol or thiol containing side chain.

P Embodiment 13. The pharmaceutical composition of P embodiment 1, wherein the peptide comprises an amino acid comprising a side chain that is at least partially deprotonated at a pH of about 7.3.

P Embodiment 14. The pharmaceutical composition of P embodiment 1, wherein the peptide or salt thereof comprises a recombinant peptide.

P Embodiment 15. The pharmaceutical composition of P embodiment 1, wherein at least one amino acid of the peptide or salt thereof comprises a chemical modification.

P Embodiment 16. The pharmaceutical composition of P embodiment 27, wherein the chemical modification comprises: acetylation, sulfonation, amidation, esterification, or any combination thereof.

P Embodiment 17. The pharmaceutical composition of P embodiment 1, wherein the peptide or salt thereof comprises a stapled peptide or salt thereof, a stitched peptide or salt thereof, a macrocyclic peptide or salt thereof, or any combination thereof.

P Embodiment 18. The pharmaceutical composition of P embodiment 29, comprising the stapled peptide, wherein the stapled peptide comprises a covalent linkage between two amino acid side-chains.

P Embodiment 19. The pharmaceutical composition of P embodiment 1, wherein the peptide or salt thereof further comprises a cell penetrating peptide, and wherein the cell penetrating peptide is directly or indirectly linked to the peptide or salt thereof.

P Embodiment 20. The pharmaceutical composition of P embodiment 1, wherein an amino acid of the peptide or salt thereof positioned at an end terminus comprises a side chain that can be at least partially deprotonated at a pH of about 7.3.

P Embodiment 21. A nucleic acid at least partially encoding: a peptide, having at least about 80% sequence identity to a polypeptide of any one of the sequences provided herein, and wherein the peptide: (i) modulates an expression level of a target protein implicated in a disease or condition, as measured by an at least partial increase or an at least partial decrease of a level of the target protein in an in vitro assay in a cell treated with the nucleic acid as determined by a Western blot relative to a level of the target protein in an otherwise comparable cell not treated with the nucleic acid; (ii) produces an at least partial increase or an at least partial decrease of an activity of the target protein, as measured by a level of the activity of the target protein in a cell treated with the nucleic acid relative to a level of activity of the target protein in an otherwise comparable cell not treated with the nucleic acid in as determined by an in vitro assay; (iii) produces an at least partial increase or an at least partial decrease of an activity of a protein downstream of the target protein in a cellular pathway in a cell treated with the nucleic acid relative to a level of activity of the protein downstream of the target protein in a cellular pathway in an otherwise comparable cell not treated with the nucleic acid as determined by an in vitro assay; (iv) kills a cancer cell in an in vitro assay; or (v) any combination thereof.

P Embodiment 22. The nucleic acid of P embodiment 21, wherein the peptide at least partially encoded by the nucleic acid does not comprise more than about 40 amino acids.

P Embodiment 23. The nucleic acid of P embodiment 21, wherein the nucleic acid is comprised in a pharmaceutical composition in unit dose form.

P Embodiment 24. The nucleic acid of P embodiment 21, wherein the peptide at least partially encoded by the nucleic acid comprises independently Gly, or an amino acid comprising a C1-C10 alkyl, a C1-C10 alkenyl, a C1-C10 alkynyl, a cycloalkyl, or an alkylcycloalkyl side chain.

P Embodiment 25. The nucleic acid of P embodiment 21, wherein the peptide at least partially encoded by the nucleic acid comprises an amino acid comprising an aromatic side chain.

P Embodiment 26. The nucleic acid of P embodiment 21, wherein the peptide at least partially encoded by the nucleic acid comprises an amino acid comprising a side chain that is at least partially protonated at a pH of about 7.3.

P Embodiment 27. The nucleic acid of P embodiment 21, wherein the peptide at least partially encoded by the nucleic acid comprises an amino acid comprising an amide containing side chain.

P Embodiment 28. The nucleic acid of P embodiment 21, wherein the peptide at least partially encoded by the nucleic acid comprises an amino acid comprising an alcohol or thiol containing side chain.

P Embodiment 29. The nucleic acid of P embodiment 21, wherein the peptide at least partially encoded by the nucleic acid comprises an amino acid comprising a side chain that is at least partially deprotonated at a pH of about 7.3.

P Embodiment 30. The nucleic acid of P embodiment 21, wherein the nucleic acid is double stranded.

P Embodiment 31. The nucleic acid of P embodiment 21, wherein the nucleic acid comprises DNA, RNA, or any combination thereof.

P Embodiment 32. A vector that comprises the nucleic acid of any one of P embodiments 21-31.

P Embodiment 33. The vector of P embodiment 32, wherein the vector comprises a polypeptide coat.

P Embodiment 34. The vector of P embodiment 32, wherein the vector comprises: a nanoparticle, a microparticle, a viral vector, a virus-like particle, a liposome, or any combination thereof.

P Embodiment 35. The vector of P embodiment 34, wherein the vector comprises the viral vector, and wherein the viral vector comprises an AAV vector.

P Embodiment 36. An isolated peptide or salt thereof that comprises a sequence having at least about 80% sequence homology to any one of the peptides provided herein.

P Embodiment 37. A method of at least partially treating or preventing a disease or condition in a subject, the method comprising: administering to the subject a therapeutically effective amount of: (a) the pharmaceutical composition of any one of P embodiments 1-20; (b) the nucleic acid of any one of P embodiments 21-31; (c) the vector of any one of P embodiments 32-35; (d) the isolated peptide or salt thereof of P embodiment 36; or (e) any combination of (a)-(d), thereby at least partially preventing or treating the disease or condition in the subject.

P Embodiment 38. The isolated peptide or salt there of P embodiment 36, further comprising a detectable label attached to the peptide.

### EMBODIMENTS

Embodiment 1. A peptide comprising an amino acid sequence having at least 80% sequence identity to the amino acid sequence of any one of SEQ ID NOs: 1-2327.

Embodiment 2. The peptide of embodiment 1, wherein the peptide comprises an unnatural amino acid or a stabilization moiety.

Embodiment 3. The peptide of embodiment 1 or 2, wherein the peptide comprises an amino acid sequence having at least 95% sequence identity to the amino acid sequence of any one of SEQ ID NOs: 1-2327.

Embodiment 4. The peptide of any one of embodiments 1-3, wherein the peptide comprises the amino acid sequence of any one of SEQ ID NOs: 1-2327.

Embodiment 5. The peptide of any one of embodiments 1-4, wherein the peptide comprises an amino acid comprising a side chain that is at least partially protonated at a pH of about 7.3.

Embodiment 6. The peptide of any one of embodiments 1-5, wherein the peptide comprises an amino acid comprising an alcohol or thiol containing side chain.

Embodiment 7. The peptide of any one of embodiments 1-6, wherein the peptide comprises an amino acid comprising a side chain that is at least partially deprotonated at a pH of about 7.3.

Embodiment 8. The peptide of any one of embodiments 1-7, wherein the stabilization moiety comprises a cyclization moiety, a PEGylation moiety, a glycosylation moiety, a lipidation moiety, a hydroxylation moiety, a methylation moiety, an acetylation moiety, a sulfonation moiety, an amidation moiety, an esterification moiety, an N-terminus peptide cap moiety, or a C-terminus peptide cap moiety.

Embodiment 9. The peptide of embodiment 8, wherein the cyclization moiety comprises a head-to-tail cyclization moiety or a side-chain cyclization moiety.

Embodiment 10. The peptide of embodiment 8 or 9, wherein the cyclization moiety comprises hydrocarbon stapling.

Embodiment 11. The peptide of any one of embodiments 1-10, wherein the peptide is between about 40 amino acids to about 120 amino acids in length.

Embodiment 12. The peptide of any one of embodiments 1-11, further comprising a cell-penetrating peptide.

Embodiment 13. The peptide of embodiment 12, wherein the cell-penetrating peptide comprises the amino acid sequence of any one of SEQ ID NOs: 2328-2342.

Embodiment 14. An isolated nucleic acid encoding the peptide of any one of embodiments 1-13.

Embodiment 15. An expression vector comprising the nucleic acid of embodiment 14.

Embodiment 16. A pharmaceutical composition comprising: (i) the peptide of any one of embodiments 1-13, the nucleic acid of embodiment 14, or the expression vector of embodiment 15; and (ii) a pharmaceutically acceptable excipient.

Embodiment 17. A kit comprising the peptide of any one of embodiments 1-13, the nucleic acid of embodiment 14, the expression vector of embodiment 15, the pharmaceutical composition of embodiment 16, or a combination thereof.

Embodiment 18. A method of treating a cancer in a subject in need thereof, the method comprising administering to the subject a therapeutically effective amount of the peptide of any one of embodiments 1-13, thereby treating the cancer in the subject.

Embodiment 19. The method of embodiment 18, further comprising administering an anticancer therapeutic.

What is claimed is:

1. A peptide comprising an amino acid sequence having at least 80% sequence identity to the amino acid sequence of any one of SEQ ID NOs: 1-2327.

2. The peptide of claim 1, wherein the peptide comprises an unnatural amino acid or a stabilization moiety.

3. The peptide of claim 1, wherein the peptide comprises an amino acid sequence having at least 95% sequence identity to the amino acid sequence of any one of SEQ ID NOs: 1-2327.

4. The peptide of claim 1, wherein the peptide comprises the amino acid sequence of any one of SEQ ID NOs: 1-2327.

5. The peptide of any one of claim 1, wherein the peptide comprises an amino acid comprising a side chain that is at least partially protonated at a pH of about 7.3.

6. The peptide of claim 1, wherein the peptide comprises an amino acid comprising an alcohol or thiol containing side chain.

7. The peptide of claim 1, wherein the peptide comprises an amino acid comprising a side chain that is at least partially deprotonated at a pH of about 7.3.

8. The peptide of claim 1, wherein the stabilization moiety is a cyclization moiety, a PEGylation moiety, a glycosylation moiety, a lipidation moiety, a hydroxylation moiety, a methylation moiety, an acetylation moiety, a sulfonation moiety, an amidation moiety, an esterification moiety, an N-terminus peptide cap moiety, or a C-terminus peptide cap moiety.

9. The peptide of claim 8, wherein the cyclization moiety comprises a head-to-tail cyclization moiety or a side-chain cyclization moiety.

10. The peptide of claim 8, wherein the cyclization moiety comprises hydrocarbon stapling.

11. The peptide of claim 1, wherein the peptide is from about 40 amino acids to about 120 amino acids in length.

12. The peptide of claim 1, further comprising a cell-penetrating peptide.

13. The peptide of claim 12, wherein the cell-penetrating peptide comprises the amino acid sequence of any one of SEQ ID NOs: 2328-2342.

14. An isolated nucleic acid encoding the peptide of claim 1.

15. An expression vector comprising the nucleic acid of claim 14.

16. A pharmaceutical composition comprising: (i) the peptide of claim 1, the nucleic acid of claim 14, or the expression vector of claim 15; and (ii) a pharmaceutically acceptable excipient.

17. A kit comprising the peptide of claim 1, the nucleic acid of claim 14, the expression vector of claim 15, the pharmaceutical composition of claim 16, or a combination thereof.

18. A method of treating a cancer in a subject in need thereof, the method comprising administering to the subject a therapeutically effective amount of the peptide of claim 1, thereby treating the cancer in the subject.

19. The method of claim 18, further comprising administering an anticancer therapeutic.
